(19) United States
(12) Patent Application Publication
(10) Pub. No.: US 2025/0215010 A1
(43) Pub. Date: Jul. 3, 2025

(54) DIAZABICYCLOOCTANE DERIVATIVES USEFUL AS MATRIX METALLOPROTEINASE INHIBITORS

(71) Applicant: VASA THERAPEUTICS SPOLKA Z OGRANICZONA ODPOWIEDZIALNOSCIA, Wroclaw (PL)

(72) Inventors: Piotr Jakub LIPINSKI, Kobylka (PL); Benjamin Anthony PRATT, Encinitas, CA (US)

(21) Appl. No.: 18/852,843

(22) PCT Filed: Mar. 29, 2023
(86) PCT No.: PCT/IB2023/053124

§ 371 Date: Sep. 30, 2024

Related U.S. Application Data
(60) Provisional application No. 63/324,713, filed on Mar. 29, 2022.

(30) Foreign Application Priority Data
Mar. 29, 2022 (PL) P.440789

(51) Int. Cl. C07D 487/08; A61K 31/4995; A61K 31/506; A61K 31/5377

(52) U.S. Cl. CPC C07D 487/08; A61K 31/4995; A61K 31/506; A61K 31/5377

(57) ABSTRACT

Provided herein are compounds that are MMP inhibitors, methods of making such compounds, pharmaceutical compositions and medicaments comprising such compounds, and methods of using such compounds in treating a disease, disorder or condition selected from cardiovascular disorders, lung disorders, renal disorders, hepatic disorders, and scleroderma pigmentosum. (Formula I)

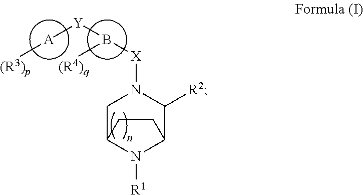

### CROSS-REFERENCE TO RELATED APPLICATIONS

This application claims the benefit of priority to Polish Patent Application number P.440789, filed Mar. 29, 2022; and U.S. Provisional Patent Application Ser. No. 63/324,713, filed Mar. 29, 2022; the contents of which are hereby incorporated by reference.

### BACKGROUND

Cardiovascular disease (CVD) is the leading cause of mortality and morbidity globally (The Global Burden of Disease: 2004 Update. In: World Health Organization; 2004). Despite improvements resulting from treatment of risk factors (Circulation. 2017; 135(10):e146.) by 2030 nearly half of the US population will suffer from CVD.

Cardiac damage, valvular heart disease such as aortic stenosis, vascular hypertension, and aging are the major factors leading to pressure overload of the pump function of the heart. Hypertrophy of the left ventricle of the heart is a normal response of the heart to such pressure overload. Hypertrophy enables cardiomyocytes to generate the additional force required to compensate for the increased pressure load and maintain necessary pump function. At least 10% of the population in industrialized societies have severe hypertension, which leads not only to left ventricular hypertrophy and secondary heart failure, but also myocardial infarctions and lethal arrhythmias (1). Aging is another common cause of pressure overload of the pump, leading to diastolic dysfunction and concentric remodeling in the left ventricle, though in many cases systolic function is unaffected until late-life senescence (2). The initially appropriate and compensatory left ventricular remodeling may progress to the point of pathophysiology, however, as ischemic and otherwise damaged cardiomyocytes degenerate, fibrosis begins and progresses, and chambers dilate, causing a progressive decline in cardiac pump function (3-9).

The maladaptive remodeling process leading to heart failure can be conceptually divided into hypertrophic changes of cardiomyocytes, and alterations to the normal patterning of the extracellular matrix (ECM) network. Changes in both systolic and diastolic function can be attributed back these two categories (10). The cardiac ECM provides structural support for the myocardium, and generates cytokines, growth factors, and modulatory proteins that modify myocardial function (11). The appearance of adverse ECM remodeling represents a significant etiologic milestone in the progression to frank heart failure. Normal basement membrane fibronectin, laminin, and collagens bind cardiomyocytes to the interstitial ECM and promote transmembrane signaling through integrin receptors (12), with collagen fibers architecturally organizing myocytes and muscle fibers to permit optimal force generation and transmission. However, when collagen fibrils and struts are lost, cardiomyocyte contraction slippage, chamber dilation, and worsening contractile dysfunction may occur (13). As well, with increasing disease promotion through worsening of the factors described above, fibrillar collagens accumulate in the left ventricular myocardium, causing increased myocardial stiffness (2, 14, 15).

The extracellular matrix of the heart, like skeletal bone and other structural components of the body, undergoes a continuous process of synthesis, degradation, and resynthesis to maintain appropriate structure and strength. Though this healthy remodeling of the cardiac ECM requires healthy proteolysis, proteolysis also is involved in adverse remodeling as part of disease processed including progressive heart failure. Of note, increased activation and expression of matrix metalloproteinases (MMPs), Zn-dependent proteases, are associated with heart failure and thus implicated in the remodeling process of progressive pump failure (16-18). There are 25 members of the mammalian MMP superfamily, including stromelysins, gelatinases, collagenases, matrilysins, membrane-type MMPs, and other MMPs (19). Their various actions are endogenously regulated by tissue inhibitors of metalloproteinases (TIMPs: TIMP1-4), which noncovalently bind with generally low selectivity to form 1:1 complexes with both active and pro-form MMPs. Each myocardial ECM protein serves as a substrate for at least one cardiac MMP (20), which also inactivate and activate ECM-derived cytokines and growth factors (21, 22).

Involvement of MMPs with pathological remodeling of myocardium leading to and worsening heart failure has been characterized in both plasma and left ventricle of animal disease models and humans (8, 16-18, 23-46). While the majority of substrates of MMPs are accessible from extracellular space, more recently an intracellular role in regulation of calcium handling important for excitation-contraction coupling and direct effects on sarcomeric function have been reported for MMPs (47-53). In humans, elevated MMP2 and MMP9 are strongly associated with diastolic dysfunction or heart failure in a number of studies (54-61).

Evidence that MMPs are not simply associated, but cause, pathological left ventricular and overall cardiac remodeling leading to reduced pump function and disease have been generated through both genetic and pharmacological interventions. Nonselective pharmacologic inhibition of MMPs has been demonstrated to significantly reduce pathological remodeling associated with various disease triggers in animal models (28, 40, 62-69). Data showing that selective MMP inhibition, in particular sparing MMP1 action, provides benefit for cardiac remodeling has been generated for MMP-1, MMP-2, MMP-9, TIMP 1, and TIMP-3 (70-74). The strongest bodies of evidence generate the therapeutic hypothesis that selective inhibition of at least MMP-2 and MMP-9 promotes less adverse cardiac function after a variety of interventions that cause cardiac pump failure (59, 75, 76).

MMP9 mice are protected from diastolic dysfunction and fibrosis accumulation (77), as well as remodeling after infarction (59)(75). MMP2 expression, as well as MMP9 expression, has been demonstrated to be elevated in pressure overload hypertrophy in both spontaneously hypertensive (66) and in Dahl salt-sensitive hypertensive rats (43). Most convincingly, both transgenically overexpressing MMP2 and transgenically overexpressing MMP9 mice develop pathologically fibrotic myocardium (78, 79), while MMP2 and MMP9 genetically deleted mice are protected structurally and functionally in disease models of heart failure, results that are nicely recapitulated with prototype pharmacological inhibitors blocking MMP2 and/or MMP9 action (80, 81).

TACE/ADAM17 (Tumor necrosis factor-α-converting enzyme; A Disintegrin And Metalloproteinase 17) is a soluble or membrane-bound metalloproteinase primarily responsible for activation of proTNF-α, while also targeting proteins such as fractalkine, amyloid precursor proteins, and CD40. ADAM17/TACE is involved in cancer, vascular disorders, and inflammatory diseases such as rheumatoid arthritis and focal ischemic injury. The catalytic domain of ADAM17/TACE is able to cleave proTNF-α and is used in inhibitor screening.

### REFERENCES

1. Vakili B A, Okin P M, Devereux R B. Prognostic implications of left ventricular hypertrophy. Am Heart J. 2001; 141(3):334-41. Epub 2001/03/07. doi: 10.1067/mhj.2001.113218. PubMed PMID: 11231428.
2. Lakatta E G, Levy D. Arterial and cardiac aging: major shareholders in cardiovascular disease enterprises: Part II: the aging heart in health: links to heart disease. Circulation. 2003; 107(2):346-54. Epub 2003/01/23. doi: 10.1161/01.cir.0000048893.62841.f7. PubMed PMID: 12538439.
3. Weber K T. Fibrosis and hypertensive heart disease. Current opinion in cardiology. 2000; 15(4):264-72.
4. Diez J, López B, Gonzilez A, Querejeta R. Clinical aspects of hypertensive myocardial fibrosis. Current opinion in cardiology. 2001; 16(6):328-35.
5. Jessup M, Greenberg B, Mancini D, Cappola T, Pauly D F, Jaski B, Yaroshinsky A, Zsebo K M, Dittrich H, Hajjar R J. Calcium Upregulation by Percutaneous Administration of Gene Therapy in Cardiac Disease (CUPID): a phase 2 trial of intracoronary gene therapy of sarcoplasmic reticulum Ca2+-ATPase in patients with advanced heart failure. Circulation. 2011; 124(3):304-13. Epub 2011/06/29. doi: 10.1161/circulationaha.111.022889. PubMed PMID: 21709064; PMCID: PMC5843948.
6. Jugdutt B I. Remodeling of the myocardium and potential targets in the collagen degradation and synthesis pathways. Current Drug Targets-Cardiovascular & Hematological Disorders. 2003; 3(1):1-30.
7. Katz A M. Pathophysiology of heart failure: identifying targets for pharmacotherapy. Medical Clinics. 2003; 87(2):303-16.
8. Janicki J S, Brower G L, Gardner J D, Chancey A L, Stewart J A. The dynamic interaction between matrix metalloproteinase activity and adverse myocardial remodeling. Heart failure reviews. 2004; 9(1):33-42.
9. Jessup M, Brozena S. Heart failure. The New England journal of medicine. 2003; 348(20):2007-18. Epub 2003/05/16. doi: 10.1056/NEJMra021498. PubMed PMID: 12748317.
10. Redfield M M, Jacobsen S J, Burnett J C, Jr., Mahoney D W, Bailey K R, Rodeheffer R J. Burden of systolic and diastolic ventricular dysfunction in the community: appreciating the scope of the heart failure epidemic. Jama. 2003; 289(2):194-202. Epub 2003/01/09. doi: 10.1001/jama.289.2.194. PubMed PMID: 12517230.
11. Jacob M P. Extracellular matrix remodeling and matrix metalloproteinases in the vascular wall during aging and in pathological conditions. Biomedicine & pharmacotherapy. 2003; 57(5-6):195-202.
12. Ross R S. The extracellular connections: the role of integrins in myocardial remodeling. Journal of cardiac failure. 2002; 8(6):S326-S31.
13. Janicki J S, Brower G L. The role of myocardial fibrillar collagen in ventricular remodeling and function. Journal of cardiac failure. 2002; 8(6):S319-S25.
14. Ferrari A U, Radaelli A, Centola M. Invited review: aging and the cardiovascular system. Journal of applied physiology (Bethesda, Md: 1985). 2003; 95(6):2591-7. Epub 2003/11/06. doi: 10.1152/japplphysiol.00601.2003. PubMed PMID: 14600164.
15. Martos R, Baugh J, Ledwidge M, O'Loughlin C, Conlon C, Patle A, Donnelly S C, McDonald K. Diastolic heart failure: evidence of increased myocardial collagen turnover linked to diastolic dysfunction. Circulation. 2007; 115(7):888-95. Epub 2007/02/07. doi: 10.1161/circulationaha.106.638569. PubMed PMID: 17283265.
16. Spinale F G. Matrix metalloproteinases: regulation and dysregulation in the failing heart. Circulation research. 2002; 90(5):520-30.
17. DeLeon-Pennell K Y, Meschiari C A, Jung M, Lindsey M L. Matrix metalloproteinases in myocardial infarction and heart failure. Progress in molecular biology and translational science. 2017; 147:75-100.
18. Spinale F G. Myocardial matrix remodeling and the matrix metalloproteinases: influence on cardiac form and function. Physiological reviews. 2007; 87(4):1285-342.
19. Iyer R P, Patterson N L, Fields G B, Lindsey M L. The history of matrix metalloproteinases: milestones, myths, and misperceptions. American Journal of Physiology—Heart and Circulatory Physiology. 2012; 303(8):H919-H30.
20. Brauer P R. MMPs—role in cardiovascular development and disease. Frontiers in bioscience: a journal and virtual library. 2006; 11:447-78.
21. Sternlicht M D, Werb Z. How matrix metalloproteinases regulate cell behavior. Annual review of cell and developmental biology. 2001; 17:463-516. Epub 2001/11/01. doi: 10.1146/annurev.cellbio.17.1.463. PubMed PMID: 11687497; PMCID: PMC2792593.
22. Van den Steen P E, Dubois B, Nelissen I, Rudd P M, Dwek R A, Opdenakker G. Biochemistry and molecular biology of gelatinase B or matrix metalloproteinase-9 (MMP-9). Critical reviews in biochemistry and molecular biology. 2002; 37(6):375-536.
23. Dixon J A, Spinale F G. Myocardial remodeling: cellular and extracellular events and targets. Annual review of physiology. 2011; 73:47-68.
24. Spinale F G, Villarreal F. Targeting matrix metalloproteinases in heart disease: lessons from endogenous inhibitors. Biochemical pharmacology. 2014; 90(1):7-15.
25. Spinale F G, Zile M R. Integrating the myocardial matrix into heart failure recognition and management. Circulation research. 2013; 113(6):725-38.
26. Cleutjens J P, Creemers E E. Integration of concepts: cardiac extracellular matrix remodeling after myocardial infarction. Journal of cardiac failure. 2002; 8(6):S344-S8.
27. Iwanaga Y, Aoyama T, Kihara Y, Onozawa Y, Yoneda T, Sasayama S. Excessive activation of matrix metalloproteinases coincides with left ventricular remodeling during transition from hypertrophy to heart failure in hypertensive rats. Journal of the American College of Cardiology. 2002; 39(8):1384-91.
28. Li Y Y, McTiernan C F, Feldman A M. Interplay of matrix metalloproteinases, tissue inhibitors of metalloproteinases and their regulators in cardiac matrix remodeling. Cardiovascular research. 2000; 46(2):214-24.
29. Nagatomo Y, Carabello B A, Coker M L, McDermott P J, Nemoto S, Hamawaki M, Spinale F G. Differential effects of pressure or volume overload on myocardial MMP levels and inhibitory control. American Journal of Physiology-Heart and Circulatory Physiology. 2000; 278(1):H151-H61.
30. Peterson J T, Li H, Dillon L, Bryant J W. Evolution of matrix metalloprotease and tissue inhibitor expression during heart failure progression in the infarcted rat. Cardiovascular research. 2000; 46(2):307-15.
31. Reinhardt D, Sigusch H, Henpe J, Tyagi S, Korfer R, Figulla H. Cardiac remodelling in end stage heart failure: upregulation of matrix metalloproteinase (MMP) irrespective of the underlying disease, and evidence for a direct inhibitory effect of ACE inhibitors on MMP. Heart. 2002; 88(5):525-30.
32. Shastry S, Hayden M R, Lucchesi P A, Tyagi S C. Matrix metalloproteinase in left ventricular remodeling and heart failure. Current cardiology reports. 2003; 5(3):200-4.
33. Spinale F G, Coker M L, Heung L J, Bond B R, Gunasinghe H R, Etoh T, Goldberg A T, Zellner J L, Crumbley A J. A matrix metalloproteinase induction/activation system exists in the human left ventricular myocardium and is upregulated in heart failure. Circulation. 2000; 102(16):1944-9.
34. Spinale F G, Coker M L, Thomas C V, Walker J D, Mukherjee R, Hebbar L. Time-dependent changes in matrix metalloproteinase activity and expression during the progression of congestive heart failure: relation to ventricular and myocyte function. Circulation research. 1998; 82(4):482-95.
35. Thomas C V, Coker M L, Zellner J L, Handy J R, Crumbley III AJ, Spinale F G. Increased matrix metalloproteinase activity and selective upregulation in LV myocardium from patients with end-stage dilated cardiomyopathy. Circulation. 1998; 97(17):1708-15.
36. Chapman R E, Spinale F G. Extracellular protease activation and unraveling of the myocardial interstitium: critical steps toward clinical applications. American Journal of Physiology-Heart and Circulatory Physiology. 2004; 286(1):H1-H10.
37. Fedak P W, Altamentova S M, Weisel R D, Nili N, Ohno N, Verma S, Lee T-YJ, Kiani C, Mickle D A, Strauss B H. Matrix remodeling in experimental and human heart failure: a possible regulatory role for TIMP-3. American Journal of Physiology-Heart and Circulatory Physiology. 2003; 284(2):H626-H34.
38. Lindsey M L. MMP induction and inhibition in myocardial infarction. Heart failure reviews. 2004; 9(1):7-19.
39. Lindsey M L, Mann D L, Entman M L, Spinale F G. Extracellular matrix remodeling following myocardial injury. Annals of medicine. 2003; 35(5):316-26.
40. Miura S, Ohno I, Suzuki J, Suzuki K, Okada S, Okuyama A, Nawata J, Ikeda J, Shirato K. Inhibition of matrix metalloproteinases prevents cardiac hypertrophy induced by β-adrenergic stimulation in rats. Journal of cardiovascular pharmacology. 2003; 42(2):174-81.
41. Nishikawa N, Yamamoto K, Sakata Y, Mano T, Yoshida J, Miwa T, Takeda H, Hori M, Masuyama T. Differential activation of matrix metalloproteinases in heart failure with and without ventricular dilatation. Cardiovascular research. 2003; 57(3):766-74.
42. Pauschinger M, Chandrasekharan K, Schultheiss H-P. Myocardial remodeling in viral heart disease: possible interactions between inflammatory mediators and MMP-TIMP system. Heart failure reviews. 2004; 9(1):21-31.
43. Sakata Y, Yamamoto K, Mano T, Nishikawa N, Yoshida J, Hori M, Miwa T, Masuyama T. Activation of matrix metalloproteinases precedes left ventricular remodeling in hypertensive heart failure rats: its inhibition as a primary effect of angiotensin-converting enzyme inhibitor. Circulation. 2004; 109(17):2143-9.
44. Sierevogel M, Pasterkamp G, De Kleijn D, Strauss B. Matrix metalloproteinases: a therapeutic target in cardiovascular disease. Current pharmaceutical design. 2003; 9(13):1033-40.
45. Tao Z-Y, Cavasin M A, Yang F, Liu Y-H, Yang X-P. Temporal changes in matrix metalloproteinase expression and inflammatory response associated with cardiac rupture after myocardial infarction in mice. Life sciences. 2004; 74(12):1561-72.
46. Wilson E M, Moainie S L, Baskin J M, Lowry A S, Deschamps A M, Mukherjee R, Guy T S, St John-Sutton M G, Gorman III J H, Edmunds Jr L H. Region- and type-specific induction of matrix metalloproteinases in post-myocardial infarction remodeling. Circulation. 2003; 107(22):2857-63.
47. Ali M A, Cho W J, Hudson B, Kassiri Z, Granzier H, Schulz R. Titin is a target of matrix metalloproteinase-2: implications in myocardial ischemia/reperfusion injury. Circulation. 2010; 122(20):2039-47. Epub 2010/11/03. doi: 10.1161/circulationaha.109.930222. PubMed PMID: 21041693; PMCID: PMC3057897.
48. Ali M A, Fan X, Schulz R. Cardiac sarcomeric proteins: novel intracellular targets of matrix metalloproteinase-2 in heart disease. Trends in cardiovascular medicine. 2011; 21(4):112-8. Epub 2012/06/12. doi: 10.1016/j.tcm.2012.03.008. PubMed PMID: 22681966.
49. Fan X, Hughes B G, Ali M A, Chan B Y, Launier K, Schulz R. Matrix metalloproteinase-2 in oncostatin M-induced sarcomere degeneration in cardiomyocytes. American journal of physiology Heart and circulatory physiology. 2016; 311(1):H183-9. Epub 2016/05/21. doi: 10.1152/ajpheart.00229.2016. PubMed PMID: 27199120.
50. Lovett D H, Chu C, Wang G, Ratcliffe M B, Baker A J. A N-terminal truncated intracellular isoform of matrix metalloproteinase-2 impairs contractility of mouse myocardium. Front Physiol. 2014; 5:363. Epub 2014/10/14. doi: 10.3389/fphys.2014.00363. PubMed PMID: 25309453; PMCID: PMC4174733.
51. Rouet-Benzineb P, Buhler J M, Dreyfus P, Delcourt A, Dorent R, Perennec J, Crozatier B, Harf A, Lafuma C. Altered balance between matrix gelatinases (MMP-2 and MMP-9) and their tissue inhibitors in human dilated cardiomyopathy: potential role of MMP-9 in myosin-heavy chain degradation. European journal of heart failure. 1999; 1(4):337-52. Epub 2000/08/11. doi: 10.1016/s1388-9842(99)00048-3. PubMed PMID: 10937947.
52. Sawicki G, Leon H, Sawicka J, Sariahmetoglu M, Schulze C J, Scott P G, Szczesna-Cordary D, Schulz R. Degradation of myosin light chain in isolated rat hearts subjected to ischemia-reperfusion injury: a new intracellular target for matrix metalloproteinase-2. Circulation. 2005; 112(4):544-52. Epub 2005/07/20. doi: 10.1161/circulationaha.104.531616. PubMed PMID: 16027249.
53. Wang G Y, Bergman M R, Nguyen A P, Turcato S, Swigart P M, Rodrigo M C, Simpson P C, Karliner J S, Lovett D H, Baker A J. Cardiac transgenic matrix metalloproteinase-2 expression directly induces impaired contractility. Cardiovasc Res. 2006; 69(3):688-96. Epub 2005/09/27. doi: 10.1016/j.cardiores.2005.08.023. PubMed PMID: 16183043.
54. Chancey A L, Brower G L, Peterson J T, Janicki J S. Effects of matrix metalloproteinase inhibition on ventricular remodeling due to volume overload. Circulation. 2002; 105(16):1983-8.
55. Cox M J, Hawkins U A, Hoit B D, Tyagi S C. Attenuation of oxidative stress and remodeling by cardiac inhibitor of metalloproteinase protein transfer. Circulation. 2004; 109(17):2123-8.
56. McMurray J J, Packer M, Desai A S, Gong J, Lefkowitz M P, Rizkala A R, Rouleau J L, Shi V C, Solomon S D, Swedberg K, Zile M R. Angiotensin-neprilysin inhibition versus enalapril in heart failure. The New England journal of medicine. 2014; 371(11):993-1004. Epub 2014/09/02. doi: 10.1056/NEJMoa1409077. PubMed PMID: 25176015.
57. Mukherjee R, Brinsa T A, Dowdy K B, Scott A A, Baskin J M, Deschamps A M, Lowry A S, Escobar G P, Lucas D G, Yarbrough W M. Myocardial infarct expansion and matrix metalloproteinase inhibition. Circulation. 2003; 107(4):618-25.
58. Peterson J T, Hallak H, Johnson L, Li H, O'Brien P M, Sliskovic D R, Bocan™, Coker M L, Etoh T, Spinale F G. Matrix metalloproteinase inhibition attenuates left ventricular remodeling and dysfunction in a rat model of progressive heart failure. Circulation. 2001; 103(18):2303-9.
59. Rohde L E, Ducharme A, Arroyo L H, Aikawa M, Sukhova G H, Lopez-Anaya A, McClure K F, Mitchell P G, Libby P, Lee R T. Matrix metalloproteinase inhibition attenuates early left ventricular enlargement after experimental myocardial infarction in mice. Circulation. 1999; 99(23):3063-70.
60. Spinale F G, Coker M L, Bond B R, Zellner J L. Myocardial matrix degradation and metalloproteinase activation in the failing heart: a potential therapeutic target. Cardiovascular research. 2000; 46(2):225-38.
61. Spinale F G, Coker M L, Krombach S R, Mukherjee R, Hallak H, Houck W V, Clair M J, Kribbs S B, Johnson L L, Peterson J T. Matrix metalloproteinase inhibition during the development of congestive heart failure: effects on left ventricular dimensions and function. Circulation research. 1999; 85(4):364-76.
62. King M K, Coker M L, Goldberg A, McElmurray III J H, Gunasinghe H R, Mukherjee R, Zile M R, O'Neill T P, Spinale F G. Selective matrix metalloproteinase inhibition with developing heart failure: effects on left ventricular function and structure. Circulation research. 2003; 92(2):177-85.
63. Lindsey M L, Gannon J, Aikawa M, Schoen F J, Rabkin E, Lopresti-Morrow L, Crawford J, Black S, Libby P, Mitchell P G. Selective matrix metalloproteinase inhibition reduces left ventricular remodeling but does not inhibit angiogenesis after myocardial infarction. Circulation. 2002; 105(6):753-8.
64. Villarreal F J, Griffin M, Omens J, Dillmann W, Nguyen J, Covell J. Early short-term treatment with doxycycline modulates postinfarction left ventricular remodeling. Circulation. 2003; 108(12):1487-92.
65. Yarbrough W M, Mukherjee R, Escobar G P, Mingoia J T, Sample J A, Hendrick J W, Dowdy K B, McLean J E, Lowry A S, O'Neill T P. Selective targeting and timing of matrix metalloproteinase inhibition in post-myocardial infarction remodeling. Circulation. 2003; 108(14):1753-9.
66. Kim H E, Dalal S S, Young E, Legato M J, Weisfeldt M L, D'Armiento J. Disruption of the myocardial extracellular matrix leads to cardiac dysfunction. The Journal of clinical investigation. 2000; 106(7):857-66.
67. Ducharme A, Frantz S, Aikawa M, Rabkin E, Lindsey M, Rohde L E, Schoen F J, Kelly R A, Werb Z, Libby P. Targeted deletion of matrix metalloproteinase-9 attenuates left ventricular enlargement and collagen accumulation after experimental myocardial infarction. The Journal of clinical investigation. 2000; 106(1):55-62.
68. Hayashidani S, Tsutsui H, Ikeuchi M, Shiomi T, Matsusaka H, Kubota T, Imanaka-Yoshida K, Itoh T, Takeshita A. Targeted deletion of MMP-2 attenuates early LV rupture and late remodeling after experimental myocardial infarction. American Journal of Physiology-Heart and Circulatory Physiology. 2003; 285(3):H1229-H35.
69. Heymans S, Lupu F, Terclavers S, Vanwetswinkel B, Herbert J-M, Baker A, Collen D, Carmeliet P, Moons L. Loss or inhibition of uPA or MMP-9 attenuates LV remodeling and dysfunction after acute pressure overload in mice. The American journal of pathology. 2005; 166(1):15-25.
70. Chiao Y A, Ramirez T A, Zamilpa R, Okoronkwo S M, Dai Q, Zhang J, Jin Y-F, Lindsey M L. Matrix metalloproteinase-9 deletion attenuates myocardial fibrosis and diastolic dysfunction in ageing mice. Cardiovascular research. 2012; 96(3):444-55.
71. Toba H, Cannon P L, Yabluchanskiy A, Iyer R P, D'Armiento J, Lindsey M L. Transgenic overexpression of macrophage matrix metalloproteinase-9 exacerbates age-related cardiac hypertrophy, vessel rarefaction, inflammation, and fibrosis. American journal of physiology Heart and circulatory physiology. 2017; 312(3):H375-h83. Epub 2016/12/25. doi: 10.1152/ajpheart.00633.2016. PubMed PMID: 28011588; PMCID: PMC5402013.
72. Bergman M R, Teerlink J R, Mahimkar R, Li L, Zhu B Q, Nguyen A, Dahi S, Karliner J S, Lovett D H. Cardiac matrix metalloproteinase-2 expression independently induces marked ventricular remodeling and systolic dysfunction. American journal of physiology Heart and circulatory physiology. 2007; 292(4):H1847-60. Epub 2006/12/13. doi: 10.1152/ajpheart.00434.2006. PubMed PMID: 17158653.
73. Matsusaka H, Ide T, Matsushima S, Ikeuchi M, Kubota T, Sunagawa K, Kinugawa S, Tsutsui H. Targeted deletion of matrix metalloproteinase 2 ameliorates myocardial remodeling in mice with chronic pressure overload. Hypertension (Dallas, Tex: 1979). 2006; 47(4):711-7. Epub 2006/03/01. doi: 10.1161/01.hyp.0000208840.30778.00. PubMed PMID: 16505197.
74. Heymans S, Lupu F, Terclavers S, Vanwetswinkel B, Herbert J M, Baker A, Collen D, Carmeliet P, Moons L. Loss or inhibition of uPA or MMP-9 attenuates LV remodeling and dysfunction after acute pressure overload in mice. Am J Pathol. 2005; 166(1):15-25. Epub 2005/01/06. doi: 10.1016/s0002-9440(10)62228-6. PubMed PMID: 15631996; PMCID: PMC1602291.
75. Hayashidani, S., et al. (2003). “Targeted deletion of MMP-2 attenuates early LV rupture and late remodeling after experimental myocardial infarction.” American Journal of Physiology-Heart and Circulatory Physiology 285(3): H1229-H1235.
76 Heymans, S., et al. (2005). “Loss or inhibition of uPA or MMP-9 attenuates LV remodeling and dysfunction after acute pressure overload in mice.” The American journal of pathology 166(1): 15-25.
77. Matrix metalloproteinase-9 deletion attenuates myocardial fibrosis and diastolic dysfunction in ageing mice. Chiao Y A, Ramirez T A, Zamilpa R, Okoronkwo S M, Dai Q, Zhang J, Jin Y F, Lindsey M L. Cardiovasc Res. 2012 Dec. 1; 96(3):444-55. doi: 10.1093/cvr/cvs275. Epub 2012 Aug. 22.
78. Transgenic overexpression of macrophage matrix metalloproteinase-9 exacerbates age-related cardiac hypertrophy, vessel rarefaction, inflammation, and fibrosis. Toba H, Cannon P L, Yabluchanskiy A, Iyer R P, D'Armiento J, Lindsey M L. Am J Physiol Heart Circ Physiol. 2017 Mar. 1; 312(3):H375-H383. doi: 10.1152/ajpheart.00633.2016.
79. Cardiac matrix metalloproteinase-2 expression independently induces marked ventricular remodeling and systolic dysfunction. Bergman M R, Teerlink J R, Mahimkar R, Li L, Zhu B Q, Nguyen A, Dahi S, Karliner J S, Lovett D H. Am J Physiol Heart Circ Physiol. 2007 April; 292(4):H1847-60. doi: 10.1152/ajpheart.00434.2006
80. Targeted deletion of matrix metalloproteinase 2 ameliorates myocardial remodeling in mice with chronic pressure overload. Matsusaka H, Ide T, Matsushima S, Ikeuchi M, Kubota T, Sunagawa K, Kinugawa S, Tsutsui H. Hypertension. 2006 April; 47(4):711-7. doi: 10.1161/01.HYP.0000208840.30778.00. Epub 2006 Feb. 27. PMID: 16505197
81. Loss or inhibition of uPA or MMP-9 attenuates LV remodeling and dysfunction after acute pressure overload in mice. Heymans S, Lupu F, Terclavers S, Vanwetswinkel B, Herbert J M, Baker A, Collen D, Carmeliet P, Moons L. Am J Pathol. 2005 January; 166(1):15-25. doi: 10.1016/50002-9440(10)62228-6.

There is a strong need to develop therapeutic treatment options directed specifically at reversing the underlying cause of heart failure (HF), which is an impaired pumping function of the heart. However, current standard of care for HF and other CVD does not correct the underlying defect causing loss of pump function, which is loss of functioning heart muscle cells, or cardiomyocytes. Instead, existing pharmacotherapies target non-cardiac, peripheral characteristics of the cardiovascular system, including reducing pre-load and after-load by targeting blood pressure, reducing blood volume (aldosterone antagonists, diuretics), or lowering lipids contributing to vascular disease.

Selective inhibition of matrix metalloproteinases provides a valuable therapeutic treatment option for treating various disorders related to abnormal activity of matrix metalloproteinases, including, but not limited to cardiovascular disorders, lung disorders, renal disorders, hepatic disorders, and also scleroderma pigmentosum.

The cardiovascular disorders of interest comprise, inter alia, cardiovascular disease, heart failure, congestive heart failure, heart failure with reduced ejection fraction, heart failure with reserved ejection fraction, impaired cardiac contractility, age-related cardiac hypertrophy, inflammation and fibrosis, viral myocarditis, COVID-19 myocarditis, COVID-19 related myocardial fibrosis, pressure overload hypertrophy, myocardial fibrosis, myocardial infarction, myocardial ischemia/reperfusion injury, pathological remodeling of myocardium, ECM remodeling following myocardial injury, radiation myocarditis, radiation myocardial fibrosis, chemotherapy cardiomyopathy, vessel rarefaction, aortic valve sclerosis, calcific aortic valve stenosis, aortic aneurism, abdominal aorta aneurism, giant cell arteritis, age-associated arterial fibrosis, pulmonary hypertension, and right ventricle hypertrophy.

The lung disorders in question comprise idiopathic pulmonary fibrosis, acute lung injury (ALI), acute respiratory distress syndrome (ARDS), Hermansky-Pudlak syndrome (HPS), chronic obstructive pulmonary disease (COPD), and emphysema.

The appropriate renal disorders comprise polycystic kidney disease, membranous nephropathy, diabetic nephropathy, acute kidney injury, glomerulonephritis, inherited kidney disease, and chronic allograft nephropathy, focal segmental glomerulosclerosis, minimal change disease, human immunodeficiency virus-associated nephropathy, anti-neutrophil cytoplasmic antibody-associated vasculitis, lupus nephritis, IgA nephropathy, Henoch-Schoenlein purpura, and postinfectious glomerulonephritis, membranoproliferative glomerulonephritis, cisplatin-induced renal injury, tubular injury following sepsis], acute ischemic kidney injury, contrast-induced kidney injury, acute tubular injury after ischemia and reperfusion, end-stage renal disease, and tubulointerstitial fibrosis.

The hepatic disorders of interest include alcoholic liver disease, non-alcoholic fatty liver disease, non-alcoholic steatohepatitis, steatosis, cirrhosis, hepatic ischemia and reperfusion injury, viral hepatitis, drug-induced liver injury, primary biliary cholangitis, primary sclerosing cholangitis, hemochromatosis, Wilson's disease, acute liver failure, and biliary atresia.

### BRIEF SUMMARY OF THE INVENTION

This disclosure provides, for example, compounds and compositions which are MMP inhibitors, and their use as medicinal agents, processes for their preparation, and pharmaceutical compositions that include disclosed compounds as at least one active ingredient. The disclosure also provides for the use of disclosed compounds as medicaments and/or in the manufacture of medicaments for MMP inhibition in warm-blooded animals, such as humans, for the treatment of heart failure and other CVD.

In one aspect, the invention provides a compound of Formula (I), or a pharmaceutically acceptable salt or solvate thereof:

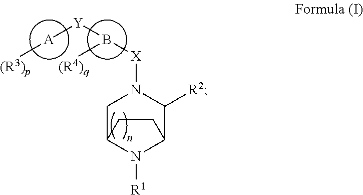

wherein:
\textcircled{A} is phenyl, C₃-C₁₀cycloalkyl, C₂-C₉heterocycloalkyl, or C₂-C₉heteroaryl;
\textcircled{B} is phenyl, C₃-C₁₀cycloalkyl, C₂-C₉heterocycloalkyl, or C₂-C₉heteroaryl;
X is —C(═O)— or —S(═O)₂—;
Y is a direct bond, —O—, —CH₂O—, —OCH₂—, —CH₂—, —C(═O)—, —C(═O)NH—, or —N(R⁵)—;
wherein Y, if asymmetrical, is written in the sequence corresponding to

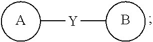

wherein if nitrogen is the attachment point on \textcircled{B} for attaching Y then Y cannot be neither —O—, —CH₂O—, —C(═O)NH—, nor —N(R⁵)—;
R¹ is hydrogen, —C₁-C₆alkyl, —C₁-C₆haloalkyl, —C₁-C₆alkylene-OR⁶, —C₁-C₆alkylene-N(R⁶)₂, —C₁-C₆alkylene-C(═O)—N(R⁶)₂, —C₁-C₆alkylene-C(═O)—C₄-C₅heterocycloalkyl, —C(═O)R⁷, —C(═O)OR⁶, —C(═O)N(R⁶)₂, —S(═O)₂R⁷, or —S(═O)₂N(R⁶)₂;
R² is —C(═O)OH, —C(═O)NH(OH), —CH₂N(OH)C(═O)R⁸, —CH(OH)N(═O), —C(═O)CF₃, —CH₂NHS(═O)₂R⁸, —CH(NH₂)C(═O)OH,

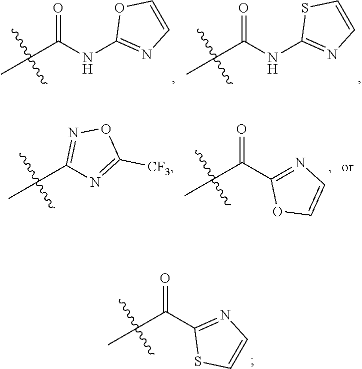 [SMILES: *C(=O)Nc1ncco1 ; *C(=O)Nc1nccs1 ; *c1noc(C(F)(F)F)n1 ; *C(=O)c1ncco1 ; *C(=O)c1nccs1]

each R³ and each R⁴ are independently selected from halogen, —C₁-C₆alkyl, —C₁-C₆haloalkyl, —C₁-C₆alkylene-OR⁶, C₁-C₆haloalkoxy, —OR⁶, —N(R⁶)₂, —CN, —C(═O)R⁷, —C(═O)OR⁶, —C(═O)N(R⁶)₂, —NR⁶C(═O)R⁷, —NR⁶SO₂R⁷, —SO₂R⁷, —SO₂N(R⁶)₂, or phenyl optionally substituted with halogen;
R⁵ is hydrogen or —C₁-C₆alkyl;
each R⁶ is independently selected from hydrogen, —C₁-C₆alkyl, —C₁-C₆haloalkyl, —C₁-C₆alkylene-OR⁹, —C₁-C₆alkylene-N(R⁹)₂, and —C₁-C₆alkylene-C₂-C₉heterocycloalkyl;
or —N(R⁶)₂ represents C₃-C₅heterocycloalkyl;
each R⁷ is independently selected from —C₁-C₆alkyl, —C₁-C₆alkylene-OR⁹, —C₁-C₆alkylene-N(R⁹)₂, —C₂-C₉heterocycloalkyl, —C₂-C₉heteroaryl, —C₁-C₆alkylene-C₂-C₉heterocycloalkyl, and —C₁-C₆alkylene-C₂-C₉heteroaryl;
R⁸ is selected from hydrogen, —C₁-C₆alkyl, and —N(H)C₁-C₆alkyl;
each R⁹ is independently selected from hydrogen and —C₁-C₆alkyl;
each occurrence of C₂-C₉heterocycloalkyl and C₂-C₉heteroaryl being optionally substituted with —C₁-C₆alkyl or —C₁-C₆haloalkyl or halogen;
n is 0, 1, or 2;
p is 0, 1, 2, or 3; and
q is 0, 1, 2, or 3.

In some embodiments, the invention provides a compound of Formula (I), or a pharmaceutically acceptable salt or solvate thereof, wherein \textcircled{A} is phenyl. In some embodiments, the invention provides a compound of Formula (I), or a pharmaceutically acceptable salt or solvate thereof, wherein \textcircled{B} is phenyl. In some embodiments, the invention provides a compound of Formula (I), or a pharmaceutically acceptable salt or solvate thereof, wherein Y is —O—. In some embodiments, the invention provides a compound of Formula (I), or a pharmaceutically acceptable salt or solvate thereof, wherein X is —S(═O)₂—. In some embodiments, the invention provides a compound of Formula (I), or a pharmaceutically acceptable salt or solvate thereof, wherein X is —C(═O)—.

In some embodiments, the invention provides a compound of Formula (I), or a pharmaceutically acceptable salt or solvate thereof, having the structure of Formula (Ia):

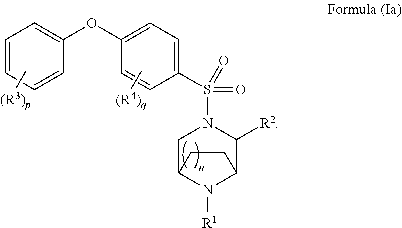

wherein:
R¹, R², R³, R⁴, n, p, and q are as defined in Formula (I).

In some embodiments, the invention provides a compound of Formula (I), or a pharmaceutically acceptable salt or solvate thereof, having the structure of Formula (Ib):

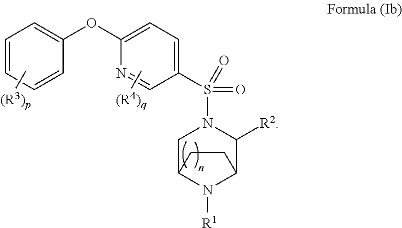

wherein:
R¹, R², R³, R⁴, n, p, and q are as defined in Formula (I).

In some embodiments, the invention provides a compound of Formula (I), or a pharmaceutically acceptable salt or solvate thereof, having the structure of Formula (Ic):

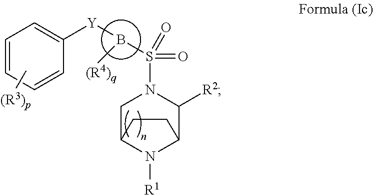

wherein:
\textcircled{B} is C₂-C₉heterocycloalkyl; and R¹, R², R³, R⁴, Y, n, p, and q are as defined in Formula (I).

In some embodiments, the invention provides a compound of Formula (I), or a pharmaceutically acceptable salt or solvate thereof, having the structure of Formula (II):

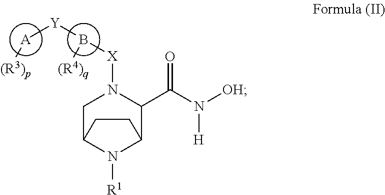

wherein:
\textcircled{A}, \textcircled{B}, R¹, R³, R⁴, X, Y, p, and q are as defined in Formula (I).

In some embodiments, the invention provides a compound of Formula (I), or a pharmaceutically acceptable salt or solvate thereof, having the structure of Formula (III):

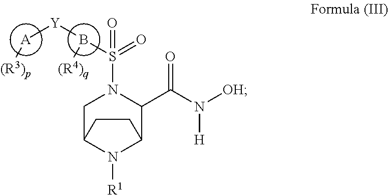

wherein:
\textcircled{A}, \textcircled{B}, R¹, R³, R⁴, Y, p, and q are as defined in Formula (I).

In some embodiments, the invention provides a compound of Formula (I), or a pharmaceutically acceptable salt or solvate thereof, having the structure of Formula (IIIa):

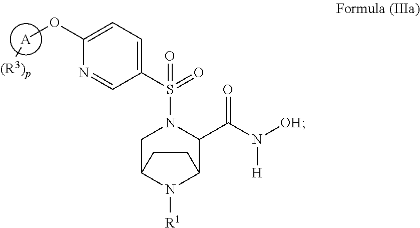

wherein:
\textcircled{A}, R¹, R³, and p are as defined in Formula (I).

In some embodiments, the invention provides a compound of Formula (I), or a pharmaceutically acceptable salt or solvate thereof, having the structure of Formula (IIIb):

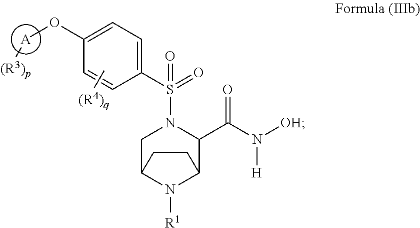

wherein:
\textcircled{A}, R¹, R³, R⁴, p, and q are as defined in Formula (I).

In some embodiments, the invention provides a compound of Formula (I), or a pharmaceutically acceptable salt or solvate thereof, having the structure of Formula (IIIc):

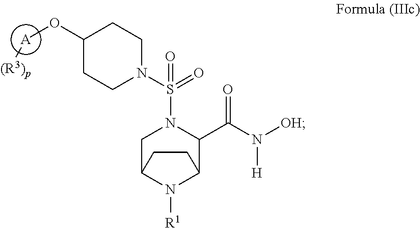

wherein:
\textcircled{A}, R¹, R³, and p are as defined in Formula (I).

In some embodiments, the invention provides a compound of Formula (I) or (Ia) or (Ib) or (Ic) or (II) or (III) or (IIIa) or (IIIb) or (IIIc), or a pharmaceutically acceptable salt or solvate thereof, wherein each R³ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, —SO₂N(R⁶)₂, and phenyl optionally substituted with halogen. In some embodiments, the invention provides a compound of Formula (I) or (Ia) or (Ib) or (Ic) or (II) or (III) or (IIIa) or (IIIb) or (IIIc), or a pharmaceutically acceptable salt or solvate thereof, wherein each R³ is independently selected from halogen, —C₁-C₆alkyl, —C₁-C₆haloalkyl, —OR⁶, and —C(═O)OR⁶. In some embodiments, the invention provides a compound of Formula (I) or (Ia) or (Ib) or (Ic) or (II) or (III) or (IIIa) or (IIIb) or (IIIc), or a pharmaceutically acceptable salt or solvate thereof, wherein each R³ is independently selected from halogen and —C₁-C₆alkyl. In some embodiments, the invention provides a compound of Formula (I) or (Ia) or (Ib) or (Ic) or (II) or (III) or (IIIa) or (IIIb) or (IIIc), or a pharmaceutically acceptable salt or solvate thereof, wherein p is 1. In some embodiments, the invention provides a compound of Formula (I) or (Ia) or (Ib) or (Ic) or (II) or (III) or (IIIa) or (IIIb) or (IIIc), or a pharmaceutically acceptable salt or solvate thereof, wherein p is 2. In some embodiments, the invention provides a compound of Formula (I) or (Ia) or (Ib) or (Ic) or (II) or (III) or (IIIa) or (IIIb) or (IIIc), or a pharmaceutically acceptable salt or solvate thereof, wherein p is 0. In some embodiments, the invention provides a compound of Formula (I) or (Ia) or (Ib) or (Ic) or (II) or (III) or (IIIb), or a pharmaceutically acceptable salt or solvate thereof, wherein each R⁴ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂, and phenyl optionally substituted with halogen. In some embodiments, the invention provides a compound of Formula (I) or (Ia) or (Ib) or (Ic) or (II) or (III) or (IIIb), or a pharmaceutically acceptable salt or solvate thereof, wherein each R⁴ is independently selected from halogen, —C₁-C₆alkyl, —C₁-C₆haloalkyl, —OR⁶, and —C(═O)OR⁶. In some embodiments, the invention provides a compound of Formula (I) or (Ia) or (Ib) or (Ic) or (II) or (III) or (IIIb), or a pharmaceutically acceptable salt or solvate thereof, wherein each R⁴ is independently selected from halogen and —C₁-C₆alkyl. In some embodiments, the invention provides a compound of Formula (I) or (Ia) or (Ib) or (Ic) or (II) or (III) or (IIIb), or a pharmaceutically acceptable salt or solvate thereof, wherein q is 1. In some embodiments, the invention provides a compound of Formula (I) or (Ia) or (Ib) or (Ic) or (II) or (III) or (IIIb), or a pharmaceutically acceptable salt or solvate thereof, wherein q is 2. In some embodiments, the invention provides a compound of Formula (I) or (Ia) or (Ib) or (Ic) or (II) or (III) or (IIIb), or a pharmaceutically acceptable salt or solvate thereof, wherein q is 0. In some embodiments, the invention provides a compound of Formula (I) or (Ia) or (Ib) or (Ic), or a pharmaceutically acceptable salt or solvate thereof, wherein R² is —C(═O)NH(OH). In some embodiments, the invention provides a compound of Formula (I) or (Ia) or (Ib) or (Ic), or a pharmaceutically acceptable salt or solvate thereof, wherein R¹ is —C(═O)OR⁶. In some embodiments, the invention provides a compound of Formula (I) or (Ia) or (Ib) or (Ic) or (II) or (III) or (IIIa) or (IIIb) or (IIIc), or a pharmaceutically acceptable salt or solvate thereof, wherein R⁶ is hydrogen, —C₁-C₆alkyl, —C₁-C₆alkylene-OR⁹, or —C₁-C₆alkylene-N(R⁹)₂. In some embodiments, the invention provides a compound of Formula (I) or (Ia) or (Ib) or (Ic) or (II) or (III) or (IIIa) or (IIIb) or (IIIc), or a pharmaceutically acceptable salt or solvate thereof, wherein R⁶ is —C₁-C₆alkylene-OR⁹. In some embodiments, the invention provides a compound of Formula (I) or (Ia) or (Ib) or (Ic) or (II) or (III) or (IIIa) or (IIIb) or (IIIc), or a pharmaceutically acceptable salt or solvate thereof, wherein R⁹ is —C₁-C₆alkyl. In some embodiments, the invention provides a compound of Formula (I) or (Ia) or (Ib) or (Ic) or (II) or (III) or (IIIa) or (IIIb) or (IIIc), or a pharmaceutically acceptable salt or solvate thereof, wherein R⁶ is —C₁-C₆alkyl. In some embodiments, the invention provides a compound of Formula (I) or (Ia) or (Ib) or (Ic), or a pharmaceutically acceptable salt or solvate thereof, wherein n is 1.

In some embodiments, the invention provides a compound of Formula (I) or (Ia) or (lb) or (Ic) or (II) or (III) or (IIIa) or (IIIb) or (IIIc) that is selected from the following compounds:

| Ex. | Structure | Name |
|---|---|---|
| 1 | 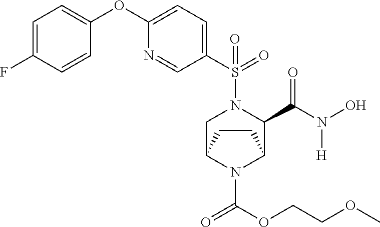 [SMILES: COCCOC(=O)N1[C@@H]2CC[C@H]1[C@H](C(=O)NO)N(S(=O)(=O)c1ccc(Oc3ccc(F)cc3)nc1)C2] | (1S,2R,5R)-2-methoxyethyl 3-((6-(4- fluorophenoxy)pyridin-3-yl)sulfonyl)-2- (hydroxycarbamoyl)-3,8- diazabicyclo[3.2.1]octane-8-carboxylate |
| 2 | 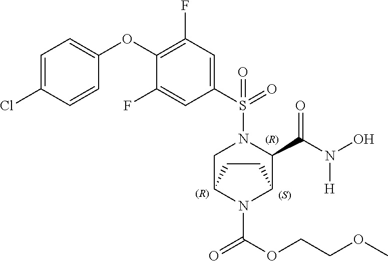 | (1S,2R,5R)-2-methoxyethyl 3-((4-(4- chlorophenoxy)-3,5- difluorophenyl)sulfonyl)-2- (hydroxycarbamoyl)-3,8- diazabicyclo[3.2.1]octane-8-carboxylate |
| 3 | 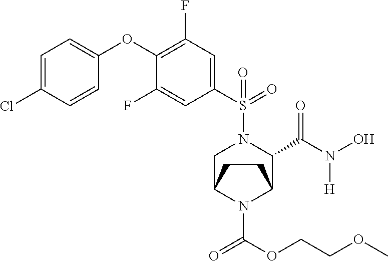 [SMILES: COCCOC(=O)N1[C@H]2CC[C@@H]1[C@@H](C(=O)NO)N(S(=O)(=O)c1cc(F)c(Oc3ccc(Cl)cc3)c(F)c1)C2] | 2-methoxyethyl (1R,2S,5S)-3-((4-(4- chlorophenoxy)-3,5- difluorophenyl)sulfonyl)-2- (hydroxycarbamoyl)-3,8- diazabicyclo[3.2.1]octane-8-carboxylate |
| 4 | 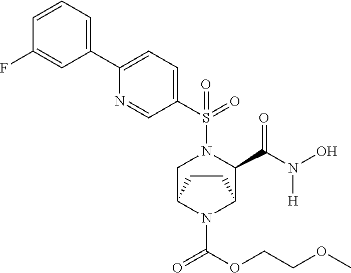 [SMILES: COCCOC(=O)N1[C@@H]2CC[C@H]1[C@H](C(=O)NO)N(S(=O)(=O)c1ccc(-c3cccc(F)c3)nc1)C2] | 2-methoxyethyl (1S,2R,5R)-3-((6-(3- fluorophenyl)pyridin-3-yl)sulfonyl)-2- (hydroxycarbamoyl)-3,8- diazabicyclo[3.2.1]octane-8-carboxylate |
| 5 | 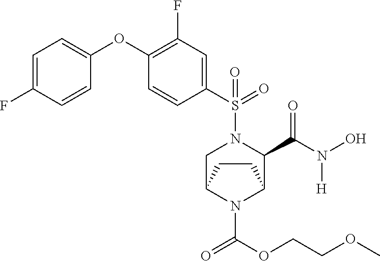 [SMILES: COCCOC(=O)N1[C@@H]2CC[C@H]1[C@H](C(=O)NO)N(S(=O)(=O)c1ccc(Oc3ccc(F)cc3)c(F)c1)C2] | 2-methoxyethyl (1S,2R,5R)-3-((3-fluoro- 4-(4-fluorophenoxy)phenyl)sulfonyl)-2- (hydroxycarbamoyl)-3,8- diazabicyclo[3.2.1]octane-8-carboxylate |
| 6 | 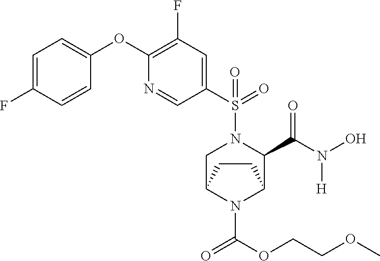 [SMILES: COCCOC(=O)N1[C@@H]2CC[C@H]1[C@H](C(=O)NO)N(S(=O)(=O)c1cnc(Oc3ccc(F)cc3)c(F)c1)C2] | 2-methoxyethyl (1S,2R,5R)-3-((5-fluoro- 6-(4-fluorophenoxy)pyridin-3- yl)sulfonyl)-2-(hydroxycarbamoyl)-3,8- diazabicyclo[3.2.1]octane-8-carboxylate |
| 7 | 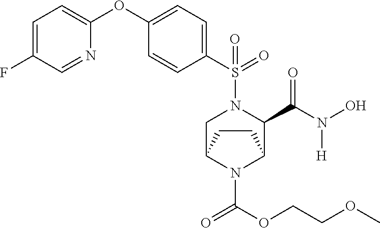 [SMILES: COCCOC(=O)N1[C@@H]2CC[C@H]1[C@H](C(=O)NO)N(S(=O)(=O)c1ccc(Oc3ccc(F)cn3)cc1)C2] | 2-methoxyethyl (1S,2R,5R)-3-((4-((5- fluoropyridin-2-yl)oxy)phenyl)sulfonyl)- 2-(hydroxycarbamoyl)-3,8- diazabicyclo[3.2.1]octane-8-carboxylate |
| 8 | 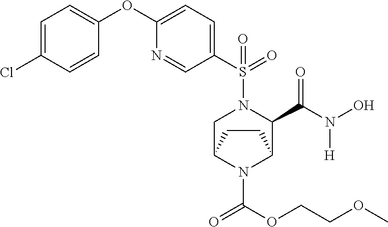 [SMILES: COCCOC(=O)N1[C@@H]2CC[C@H]1[C@H](C(=O)NO)N(S(=O)(=O)c1ccc(Oc3ccc(Cl)cc3)nc1)C2] | 2-methoxyethyl (1S,2R,5R)-3-((6-(4- chlorophenoxy)pyridin-3-yl)sulfonyl)-2- (hydroxycarbamoyl)-3,8- diazabicyclo[3.2.1]octane-8-carboxylate |
| 9 | 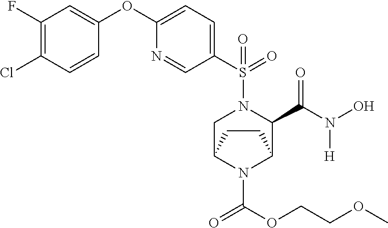 [SMILES: COCCOC(=O)N1[C@@H]2CC[C@H]1[C@H](C(=O)NO)N(S(=O)(=O)c1ccc(Oc3ccc(Cl)c(F)c3)nc1)C2] | 2-methoxyethyl (1S,2R,5R)-3-((6-(4- chloro-3-fluorophenoxy)pyridin-3- yl)sulfonyl)-2-(hydroxycarbamoyl)-3,8- diazabicyclo[3.2.1]octane-8-carboxylate |
| 10 | 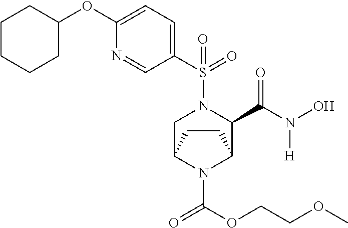 [SMILES: COCCOC(=O)N1[C@@H]2CC[C@H]1[C@H](C(=O)NO)N(S(=O)(=O)c1ccc(OC3CCCCC3)nc1)C2] | 2-methoxyethyl (1S,2R,5R)-3-((6- (cyclohexyloxy)pyridin-3-yl)sulfonyl)-2- (hydroxycarbamoyl)-3,8- diazabicyclo[3.2.1]octane-8-carboxylate |
| 11 | 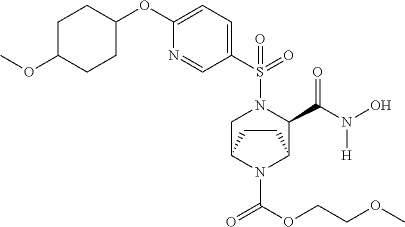 | 2-methoxyethyl (1S,2R,5R)-2- (hydroxycarbamoyl)-3-((6-((4- methoxycyclohexyl)oxy)pyridin-3- yl)sulfonyl)-3,8- diazabicyclo[3.2.1]octane-8-carboxylate |
| 12 | 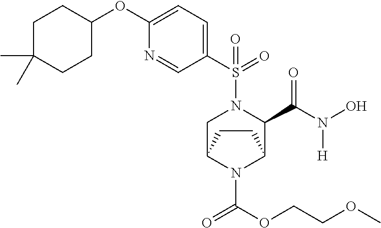 | 2-methoxyethyl (1S,2R,5R)-3-((6-((4,4- dimethylcyclohexyl)oxy)pyridin-3- yl)sulfonyl)-2-(hydroxycarbamoyl)-3,8- diazabicyclo[3.2.1]octane-8-carboxylate |
| 13 | 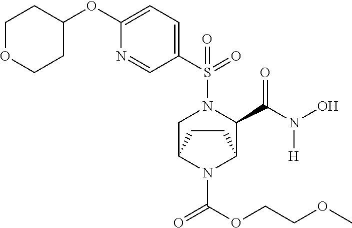 [SMILES: COCCOC(=O)N1[C@@H]2CC[C@H]1[C@H](C(=O)NO)N(S(=O)(=O)c1ccc(OC3CCOCC3)nc1)C2] | 2-methoxyethyl (1S,2R,5R)-2- (hydroxycarbamoyl)-3-((6-((tetrahydro- 2H-pyran-4-yl)oxy)pyridin-3-yl)sulfonyl)- 3,8-diazabicyclo[3.2.1]octane-8- carboxylate |
| 14 | 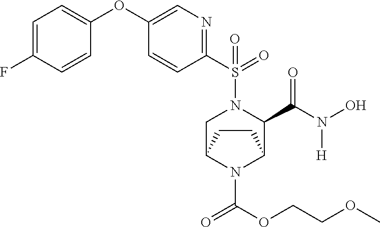 [SMILES: COCCOC(=O)N1[C@@H]2CC[C@H]1[C@H](C(=O)NO)N(S(=O)(=O)c1ccc(Oc3ccc(F)cc3)cn1)C2] | 2-methoxyethyl (1S,2R,5R)-3-((5-(4- fluorophenoxy)pyridin-2-yl)sulfonyl)-2- (hydroxycarbamoyl)-3,8- diazabicyclo[3.2.1]octane-8-carboxylate |
| 15 | 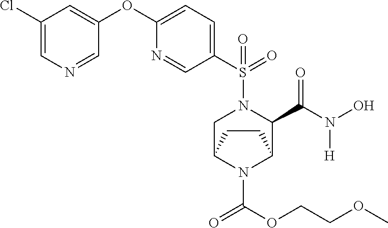 [SMILES: COCCOC(=O)N1[C@@H]2CC[C@H]1[C@H](C(=O)NO)N(S(=O)(=O)c1ccc(Oc3cncc(Cl)c3)nc1)C2] | (1S,2R,5R)-2-methoxyethyl 3-((6-((5- chloropyridin-3-yl)oxy)pyridin-3- yl)sulfonyl)-2-(hydroxycarbamoyl)-3,8- diazabicyclo[3.2.1]octane-8-carboxylate |
| 16 | 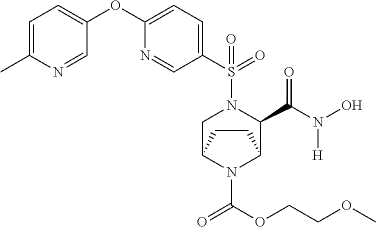 | 2-methoxyethyl (1S,2R,5R)-2- (hydroxycarbamoyl)-3-((6-((6- methylpyridin-3-yl)oxy)pyridin-3- yl)sulfonyl)-3,8- diazabicyclo[3.2.1]octane-8-carboxylate |
| 17 | 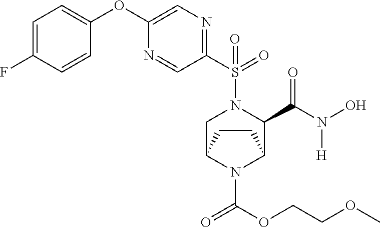 [SMILES: COCCOC(=O)N1[C@@H]2CC[C@H]1[C@H](C(=O)NO)N(S(=O)(=O)c1cnc(Oc3ccc(F)cc3)cn1)C2] | 2-methoxyethyl (1S,2R,5R)-3-((5-(4- fluorophenoxy)pyrazin-2-yl)sulfonyl)-2- (hydroxycarbamoyl)-3,8- diazabicyclo[3.2.1]octane-8-carboxylate |
| 18 | 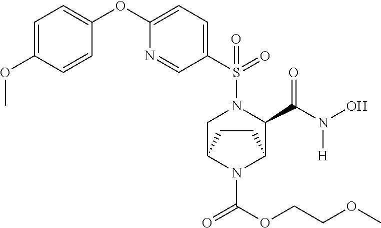 [SMILES: COCCOC(=O)N1[C@@H]2CC[C@H]1[C@H](C(=O)NO)N(S(=O)(=O)c1ccc(Oc3ccc(OC)cc3)nc1)C2] | 2-methoxyethyl (1S,2R,5R)-2- (hydroxycarbamoyl)-3-((6-(4- methoxyphenoxy)pyridin-3-yl)sulfonyl)- 3,8-diazabicyclo[3.2.1]octane-8- carboxylate |
| 19 | 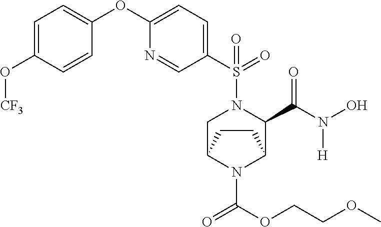 [SMILES: COCCOC(=O)N1[C@@H]2CC[C@H]1[C@H](C(=O)NO)N(S(=O)(=O)c1ccc(Oc3ccc(OC(F)(F)F)cc3)nc1)C2] | 2-methoxyethyl (1S,2R,5R)-2- (hydroxycarbamoyl)-3-((6-(4- (trifluoromethoxy)phenoxy)pyridin-3- yl)sulfonyl)-3,8- diazabicyclo[3.2.1]octane-8-carboxylate |
| 20 | 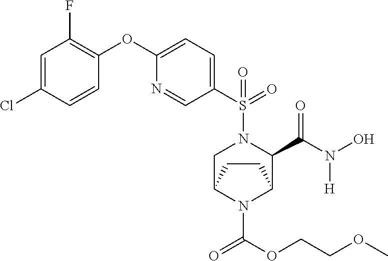 [SMILES: COCCOC(=O)N1[C@@H]2CC[C@H]1[C@H](C(=O)NO)N(S(=O)(=O)c1ccc(Oc3ccc(Cl)cc3F)nc1)C2] | 2-methoxyethyl (1S,2R,5R)-3-((6-(4- chloro-2-fluorophenoxy)pyridin-3- yl)sulfonyl)-2-(hydroxycarbamoyl)-3,8- diazabicyclo[3.2.1]octane-8-carboxylate |
| 21 | 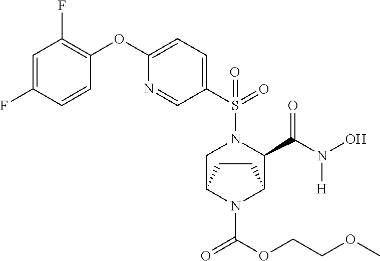 [SMILES: COCCOC(=O)N1[C@@H]2CC[C@H]1[C@H](C(=O)NO)N(S(=O)(=O)c1ccc(Oc3ccc(F)cc3F)nc1)C2] | 2-methoxyethyl (1S,2R,5R)-3-((6-(2,4- difluorophenoxy)pyridin-3-yl)sulfonyl)-2- (hydroxycarbamoyl)-3,8- diazabicyclo[3.2.1]octane-8-carboxylate |
| 22 | 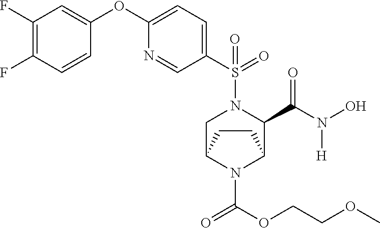 [SMILES: COCCOC(=O)N1[C@@H]2CC[C@H]1[C@H](C(=O)NO)N(S(=O)(=O)c1ccc(Oc3ccc(F)c(F)c3)nc1)C2] | 2-methoxyethyl (1S,2R,5R)-3-((6-(3,4- difluorophenoxy)pyridin-3-yl)sulfonyl)-2- (hydroxycarbamoyl)-3,8- diazabicyclo[3.2.1]octane-8-carboxylate |
| 23 | 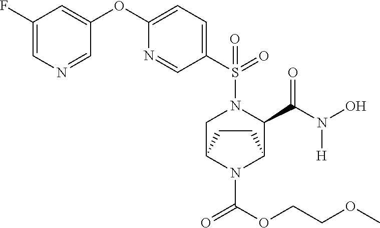 [SMILES: COCCOC(=O)N1[C@@H]2CC[C@H]1[C@H](C(=O)NO)N(S(=O)(=O)c1ccc(Oc3cncc(F)c3)nc1)C2] | 2-methoxyethyl (1S,2R,5R)-3-((6-((5- fluoropyridin-3-yl)oxy)pyridin-3- yl)sulfonyl)-2-(hydroxycarbamoyl)-3,8- diazabicyclo[3.2.1]octane-8-carboxylate |
| 24 | 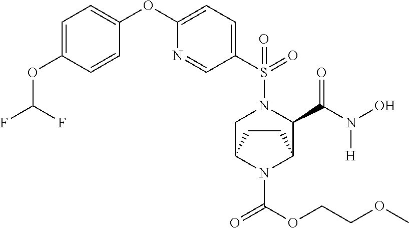 [SMILES: COCCOC(=O)N1[C@@H]2CC[C@H]1[C@H](C(=O)NO)N(S(=O)(=O)c1ccc(Oc3ccc(OC(F)F)cc3)nc1)C2] | 2-methoxyethyl (1S,2R,5R)-3-((6-(4- (difluoromethoxy)phenoxy)pyridin-3- yl)sulfonyl)-2-(hydroxycarbamoyl)-3,8- diazabicyclo[3.2.1]octane-8-carboxylate |
| 25 | 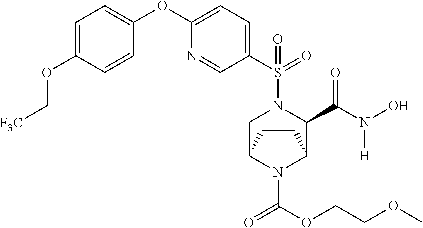 [SMILES: COCCOC(=O)N1[C@@H]2CC[C@H]1[C@H](C(=O)NO)N(S(=O)(=O)c1ccc(Oc3ccc(OCC(F)(F)F)cc3)nc1)C2] | 2-methoxyethyl (1S,2R,5R)-2- (hydroxycarbamoyl)-3-((6-(4-(2,2,2- trifluoroethoxy)phenoxy)pyridin-3- yl)sulfonyl)-3,8- diazabicyclo[3.2.1]octane-8-carboxylate |
| 26 | 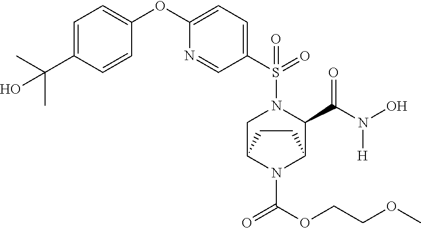 [SMILES: COCCOC(=O)N1[C@@H]2CC[C@H]1[C@H](C(=O)NO)N(S(=O)(=O)c1ccc(Oc3ccc(C(C)(C)O)cc3)nc1)C2] | 2-methoxyethyl (1S,2R,5R)-2- (hydroxycarbamoyl)-3-((6-(4-(2- hydroxypropan-2-yl)phenoxy)pyridin-3- yl)sulfonyl)-3,8- diazabicyclo[3.2.1]octane-8-carboxylate |
| 27 | 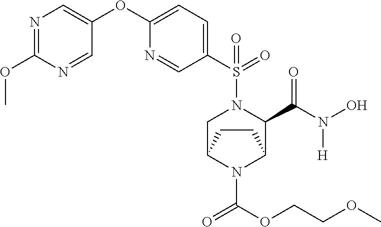 [SMILES: COCCOC(=O)N1[C@@H]2CC[C@H]1[C@H](C(=O)NO)N(S(=O)(=O)c1ccc(Oc3cnc(OC)nc3)nc1)C2] | 2-methoxyethyl (1S,2R,5R)-2- (hydroxycarbamoyl)-3-((6-((2- methoxypyrimidin-5-yl)oxy)pyridin-3- yl)sulfonyl)-3,8- diazabicyclo[3.2.1]octane-8-carboxylate |
| 28 | 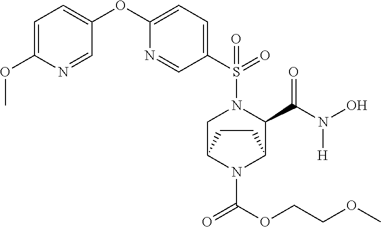 [SMILES: COCCOC(=O)N1[C@@H]2CC[C@H]1[C@H](C(=O)NO)N(S(=O)(=O)c1ccc(Oc3ccc(OC)nc3)nc1)C2] | 2-methoxyethyl (1S,2R,5R)-2- (hydroxycarbamoyl)-3-((6-((6- methoxypyridin-3-yl)oxy)pyridin-3- yl)sulfonyl)-3,8- diazabicyclo[3.2.1]octane-8-carboxylate |
| 29 | 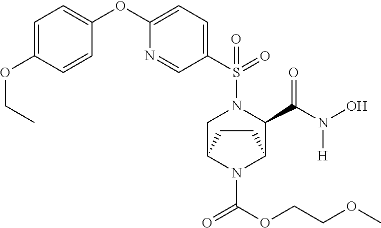 [SMILES: CCOc1ccc(Oc2ccc(S(=O)(=O)N3C[C@H]4CC[C@@H]([C@@H]3C(=O)NO)N4C(=O)OCCOC)cn2)cc1] | 2-methoxyethyl (1S,2R,5R)-3-((6-(4- ethoxyphenoxy)pyridin-3-yl)sulfonyl)-2- (hydroxycarbamoyl)-3,8- diazabicyclo[3.2.1]octane-8-carboxylate |
| 30 | 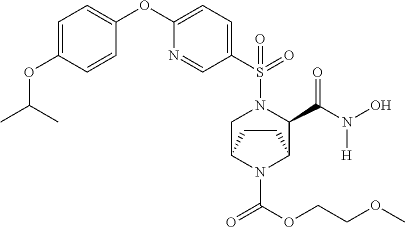 [SMILES: COCCOC(=O)N1[C@@H]2CC[C@H]1[C@H](C(=O)NO)N(S(=O)(=O)c1ccc(Oc3ccc(OC(C)C)cc3)nc1)C2] | 2-methoxyethyl (1S,2R,5R)-2- (hydroxycarbamoyl)-3-((6-(4- isopropoxyphenoxy)pyridin-3- yl)sulfonyl)-3,8- diazabicyclo[3.2.1]octane-8-carboxylate |
| 31 | 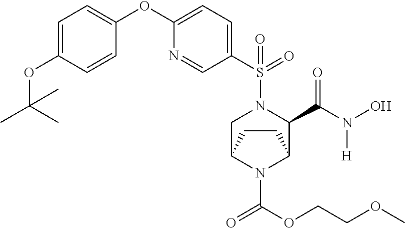 [SMILES: COCCOC(=O)N1[C@@H]2CC[C@H]1[C@H](C(=O)NO)N(S(=O)(=O)c1ccc(Oc3ccc(OC(C)(C)C)cc3)nc1)C2] | 2-methoxyethyl (1S,2R,5R)-3-((6-(4-(tert- butoxy)phenoxy)pyridin-3-yl)sulfonyl)-2- (hydroxycarbamoyl)-3,8- diazabicyclo[3.2.1]octane-8-carboxylate |
| 32 | 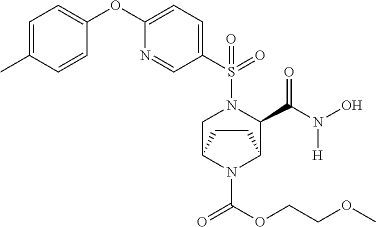 | 2-methoxyethyl (1S,2R,5R)-2- (hydroxycarbamoyl)-3-((6-(p- tolyloxy)pyridin-3-yl)sulfonyl)-3,8- diazabicyclo[3.2.1]octane-8-carboxylate |
| 33 | 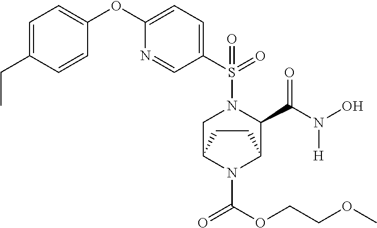 [SMILES: CCc1ccc(Oc2ccc(S(=O)(=O)N3C[C@H]4CC[C@@H]([C@@H]3C(=O)NO)N4C(=O)OCCOC)cn2)cc1] | 2-methoxyethyl (1S,2R,5R)-3-((6-(4- ethylphenoxy)pyridin-3-yl)sulfonyl)-2- (hydroxycarbamoyl)-3,8- diazabicyclo[3.2.1]octane-8-carboxylate |
| 34 | 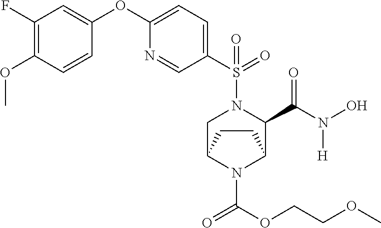 [SMILES: COCCOC(=O)N1[C@@H]2CC[C@H]1[C@H](C(=O)NO)N(S(=O)(=O)c1ccc(Oc3ccc(OC)c(F)c3)nc1)C2] | 2-methoxyethyl (1S,2R,5R)-3-((6-(3- fluoro-4-methoxyphenoxy)pyridin-3- yl)sulfonyl)-2-(hydroxycarbamoyl)-3,8- diazabicyclo[3.2.1]octane-8-carboxylate |
| 35 | 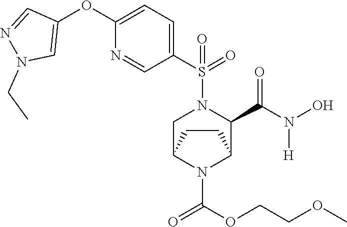 [SMILES: CCn1cc(Oc2ccc(S(=O)(=O)N3C[C@H]4CC[C@@H]([C@@H]3C(=O)NO)N4C(=O)OCCOC)cn2)cn1] | 2-methoxyethyl (1S,2R,5R)-3-((6-((1- ethyl-1H-pyrazol-4-yl)oxy)pyridin-3- yl)sulfonyl)-2-(hydroxycarbamoyl)-3,8- diazabicyclo[3.2.1]octane-8-carboxylate |
| 36 | 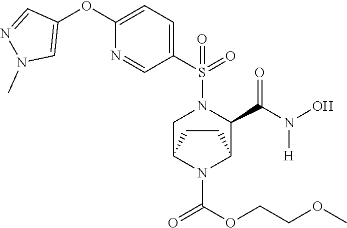 [SMILES: COCCOC(=O)N1[C@@H]2CC[C@H]1[C@H](C(=O)NO)N(S(=O)(=O)c1ccc(Oc3cnn(C)c3)nc1)C2] | 2-methoxyethyl (1S,2R,5R)-2- (hydroxycarbamoyl)-3-((6-((1-methyl-1H- pyrazol-4-yl)oxy)pyridin-3-yl)sulfonyl)- 3,8-diazabicyclo[3.2.1]octane-8- carboxylate |
| 37 | 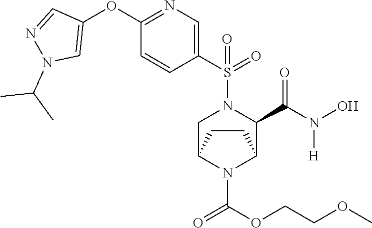 [SMILES: COCCOC(=O)N1[C@@H]2CC[C@H]1[C@H](C(=O)NO)N(S(=O)(=O)c1ccc(Oc3cnn(C(C)C)c3)nc1)C2] | 2-methoxyethyl (1S,2R,5R)-2- (hydroxycarbamoyl)-3-((6-((1-isopropyl- 1H-pyrazol-4-yl)oxy)pyridin-3- yl)sulfonyl)-3,8- diazabicyclo[3.2.1]octane-8-carboxylate |
| 38 | 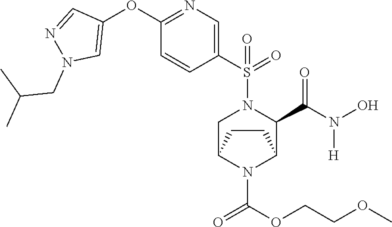 [SMILES: COCCOC(=O)N1[C@@H]2CC[C@H]1[C@H](C(=O)NO)N(S(=O)(=O)c1ccc(Oc3cnn(CC(C)C)c3)nc1)C2] | 2-methoxyethyl (1S,2R,5R)-2- (hydroxycarbamoyl)-3-((6-((1-isobutyl- 1H-pyrazol-4-yl)oxy)pyridin-3- yl)sulfonyl)-3,8- diazabicyclo[3.2.1]octane-8-carboxylate |
| 39 | 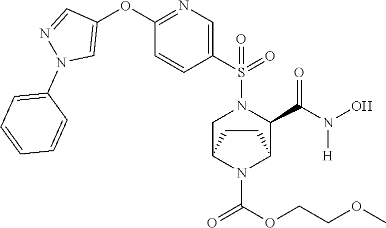 [SMILES: COCCOC(=O)N1[C@@H]2CC[C@H]1[C@H](C(=O)NO)N(S(=O)(=O)c1ccc(Oc3cnn(-c4ccccc4)c3)nc1)C2] | 2-methoxyethyl (1S,2R,5R)-2- (hydroxycarbamoyl)-3-((6-((1-phenyl-1H- pyrazol-4-yl)oxy)pyridin-3-yl)sulfonyl)- 3,8-diazabicyclo[3.2.1]octane-8- carboxylate |
| 40 | 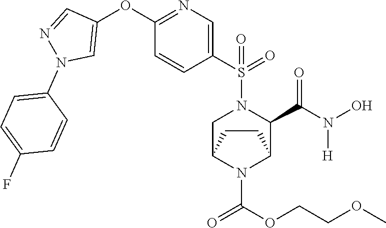 [SMILES: COCCOC(=O)N1[C@@H]2CC[C@H]1[C@H](C(=O)NO)N(S(=O)(=O)c1ccc(Oc3cnn(-c4ccc(F)cc4)c3)nc1)C2] | 2-methoxyethyl (1S,2R,5R)-3-((6-((1-(4- fluorophenyl)-1H-pyrazol-4- yl)oxy)pyridin-3-yl)sulfonyl)-2- (hydroxycarbamoyl)-3,8- diazabicyclo[3.2.1]octane-8-carboxylate |
| 41 | 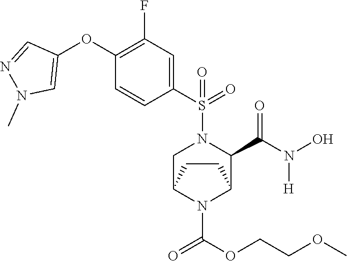 [SMILES: COCCOC(=O)N1[C@@H]2CC[C@H]1[C@H](C(=O)NO)N(S(=O)(=O)c1ccc(Oc3cnn(C)c3)c(F)c1)C2] | 2-methoxyethyl (1S,2R,5R)-3-((3-fluoro- 4-((1-methyl-1H-pyrazol-4- yl)oxy)phenyl)sulfonyl)-2- (hydroxycarbamoyl)-3,8- diazabicyclo[3.2.1]octane-8-carboxylate |
| 42 | 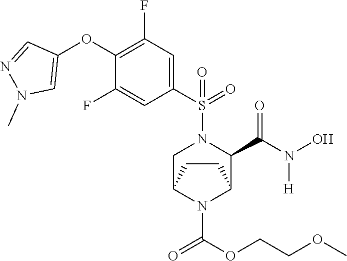 [SMILES: COCCOC(=O)N1[C@@H]2CC[C@H]1[C@H](C(=O)NO)N(S(=O)(=O)c1cc(F)c(Oc3cnn(C)c3)c(F)c1)C2] | 2-methoxyethyl (1S,2R,5R)-3-((3,5- difluoro-4-((1-methyl-1H-pyrazol-4- yl)oxy)phenyl)sulfonyl)-2- (hydroxycarbamoyl)-3,8- diazabicyclo[3.2.1]octane-8-carboxylate |
| 43 | 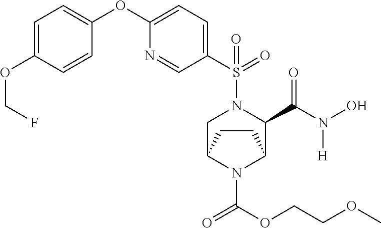 [SMILES: COCCOC(=O)N1[C@@H]2CC[C@H]1[C@H](C(=O)NO)N(S(=O)(=O)c1ccc(Oc3ccc(OCF)cc3)nc1)C2] | 2-methoxyethyl (1S,2R,5R)-3-((6-(4- (fluoromethoxy)phenoxy)pyridin-3- yl)sulfonyl)-2-(hydroxycarbamoyl)-3,8- diazabicyclo[3.2.1]octane-8-carboxylate |
| 44 | 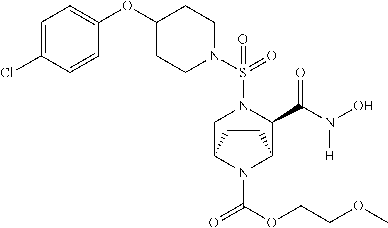 [SMILES: COCCOC(=O)N1[C@@H]2CC[C@H]1[C@H](C(=O)NO)N(S(=O)(=O)N1CCC(Oc3ccc(Cl)cc3)CC1)C2] | (1S,2R,5R)-2-methoxyethyl 3-((4-(4- chlorophenoxy)piperidin-1-yl)sulfonyl)-2- (hydroxycarbamoyl)-3,8- diazabicyclo[3.2.1]octane-8-carboxylate |
| 45 | 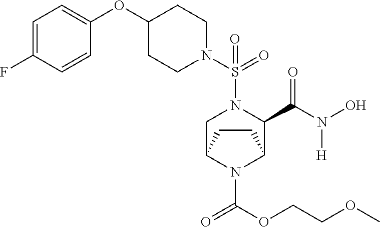 [SMILES: COCCOC(=O)N1[C@@H]2CC[C@H]1[C@H](C(=O)NO)N(S(=O)(=O)N1CCC(Oc3ccc(F)cc3)CC1)C2] | 2-methoxyethyl (1S,2R,5R)-3-((4-(4- fluorophenoxy)piperidin-1-yl)sulfonyl)-2- (hydroxycarbamoyl)-3,8- diazabicyclo[3.2.1]octane-8-carboxylate |
| 46 | 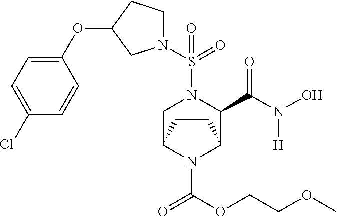 [SMILES: COCCOC(=O)N1[C@@H]2CC[C@H]1[C@H](C(=O)NO)N(S(=O)(=O)N1CCC(Oc3ccc(Cl)cc3)C1)C2] | 2-methoxyethyl (1S,2R,5R)-3-((3-(4- chlorophenoxy)pyrrolidin-1-yl)sulfonyl)- 2-(hydroxycarbamoyl)-3,8- diazabicyclo[3.2.1]octane-8-carboxylate |
| 47 | 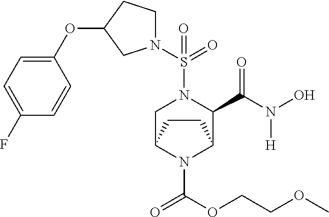 [SMILES: COCCOC(=O)N1[C@@H]2CC[C@H]1[C@H](C(=O)NO)N(S(=O)(=O)N1CCC(Oc3ccc(F)cc3)C1)C2] | 2-methoxyethyl (1S,2R,5R)-3-((3-(4- fluorophenoxy)pyrrolidin-1-yl)sulfonyl)- 2-(hydroxycarbamoyl)-3,8- diazabicyclo[3.2.1]octane-8-carboxylate |
| 48 | 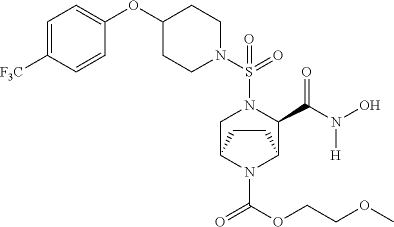 [SMILES: COCCOC(=O)N1[C@@H]2CC[C@H]1[C@H](C(=O)NO)N(S(=O)(=O)N1CCC(Oc3ccc(C(F)(F)F)cc3)CC1)C2] | 2-methoxyethyl (1S,2R,5R)-2- (hydroxycarbamoyl)-3-((4-(4- (trifluoromethyl)phenoxy)piperidin-1- yl)sulfonyl)-3,8- diazabicyclo[3.2.1]octane-8-carboxylate |
| 49 | 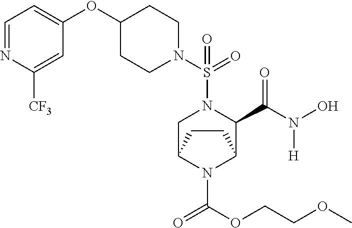 [SMILES: COCCOC(=O)N1[C@@H]2CC[C@H]1[C@H](C(=O)NO)N(S(=O)(=O)N1CCC(Oc3ccnc(C(F)(F)F)c3)CC1)C2] | 2-methoxyethyl (1S,2R,5R)-2- (hydroxycarbamoyl)-3-((4-((2- (trifluoromethyl)pyridin-4- yl)oxy)piperidin-1-yl)sulfonyl)-3,8- diazabicyclo[3.2.1]octane-8-carboxylate |
| 50 | 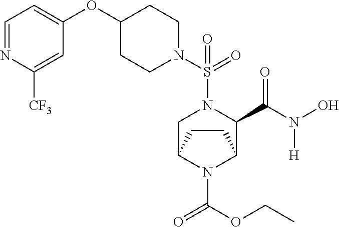 [SMILES: CCOC(=O)N1[C@@H]2CC[C@H]1[C@H](C(=O)NO)N(S(=O)(=O)N1CCC(Oc3ccnc(C(F)(F)F)c3)CC1)C2] | ethyl (1S,2R,5R)-2-(hydroxycarbamoyl)- 3-((4-((2-(trifluoromethyl)pyridin-4- yl)oxy)piperidin-1-yl)sulfonyl)-3,8- diazabicyclo[3.2.1]octane-8-carboxylate |
| 51 | 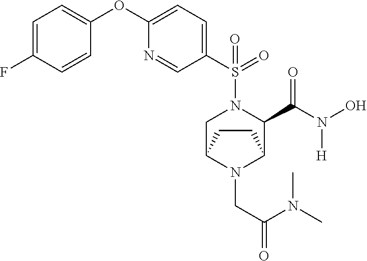 [SMILES: CN(C)C(=O)CN1[C@@H]2CC[C@H]1[C@H](C(=O)NO)N(S(=O)(=O)c1ccc(Oc3ccc(F)cc3)nc1)C2] | (1S,2R,5R)-8-(2-(dimethylamino)-2- oxoethyl)-3-((6-(4-fluorophenoxy)pyridin- 3-yl)sulfonyl)-N-hydroxy-3,8- diazabicyclo[3.2.1]octane-2-carboxamide |
| 52 | 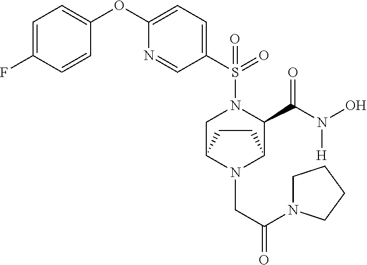 [SMILES: O=C(NO)[C@H]1[C@@H]2CC[C@H](CN1S(=O)(=O)c1ccc(Oc3ccc(F)cc3)nc1)N2CC(=O)N1CCCC1] | (1S,2R,5R)-3-((6-(4- fluorophenoxy)pyridin-3-yl)sulfonyl)-N- hydroxy-8-(2-oxo-2-(pyrrolidin-1- yl)ethyl)-3,8-diazabicyclo[3.2.1]octane-2- carboxamide |
| 53 | 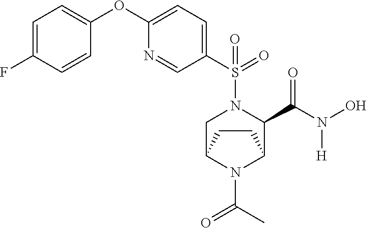 [SMILES: CC(=O)N1[C@@H]2CC[C@H]1[C@H](C(=O)NO)N(S(=O)(=O)c1ccc(Oc3ccc(F)cc3)nc1)C2] | (1S,2R,5R)-8-acetyl-3-((6-(4- fluorophenoxy)pyridin-3-yl)sulfonyl)-N- hydroxy-3,8-diazabicyclo[3.2.1]octane-2- carboxamide |
| 54 | 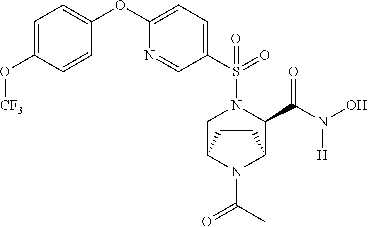 [SMILES: CC(=O)N1[C@@H]2CC[C@H]1[C@H](C(=O)NO)N(S(=O)(=O)c1ccc(Oc3ccc(OC(F)(F)F)cc3)nc1)C2] | (1S,2R,5R)-8-acetyl-N-hydroxy-3-((6-(4- (trifluoromethoxy)phenoxy)pyridin-3- yl)sulfonyl)-3,8- diazabicyclo[3.2.1]octane-2-carboxamide |
| 55 | 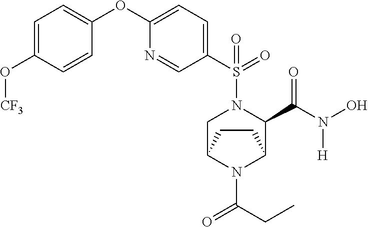 [SMILES: CCC(=O)N1[C@@H]2CC[C@H]1[C@H](C(=O)NO)N(S(=O)(=O)c1ccc(Oc3ccc(OC(F)(F)F)cc3)nc1)C2] | (1S,2R,5R)-N-hydroxy-8-propionyl-3-((6- (4-(trifluoromethoxy)phenoxy)pyridin-3- yl)sulfonyl)-3,8- diazabicyclo[3.2.1]octane-2-carboxamide |
| 56 | 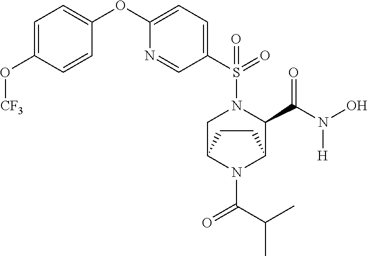 [SMILES: CC(C)C(=O)N1[C@@H]2CC[C@H]1[C@H](C(=O)NO)N(S(=O)(=O)c1ccc(Oc3ccc(OC(F)(F)F)cc3)nc1)C2] | (1S,2R,5R)-N-hydroxy-8-isobutyryl-3-((6- (4-(trifluoromethoxy)phenoxy)pyridin-3- yl)sulfonyl)-3,8- diazabicyclo[3.2.1]octane-2-carboxamide |
| 57 | 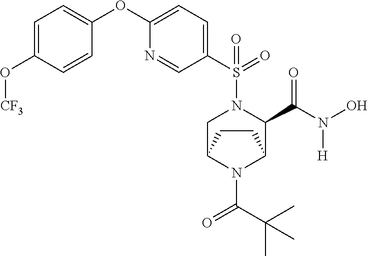 [SMILES: CC(C)(C)C(=O)N1[C@@H]2CC[C@H]1[C@H](C(=O)NO)N(S(=O)(=O)c1ccc(Oc3ccc(OC(F)(F)F)cc3)nc1)C2] | (1S,2R,5R)-N-hydroxy-8-pivaloyl-3-((6- (4-(trifluoromethoxy)phenoxy)pyridin-3- yl)sulfonyl)-3,8- diazabicyclo[3.2.1]octane-2-carboxamide |
| 58 | 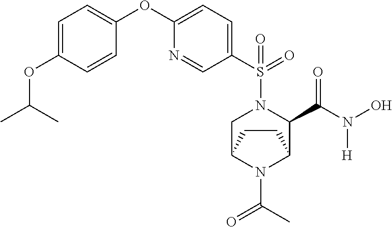 [SMILES: CC(=O)N1[C@@H]2CC[C@H]1[C@H](C(=O)NO)N(S(=O)(=O)c1ccc(Oc3ccc(OC(C)C)cc3)nc1)C2] | (1S,2R,5R)-8-acetyl-N-hydroxy-3-((6-(4- isopropoxyphenoxy)pyridin-3- yl)sulfonyl)-3,8- diazabicyclo[3.2.1]octane-2-carboxamide |
| 59 | 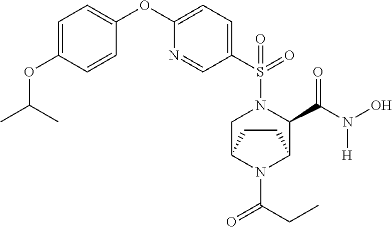 [SMILES: CCC(=O)N1[C@@H]2CC[C@H]1[C@H](C(=O)NO)N(S(=O)(=O)c1ccc(Oc3ccc(OC(C)C)cc3)nc1)C2] | (1S,2R,5R)-N-hydroxy-3-((6-(4- isopropoxyphenoxy)pyridin-3- yl)sulfonyl)-8-propionyl-3,8- diazabicyclo[3.2.1]octane-2-carboxamide |
| 60 | 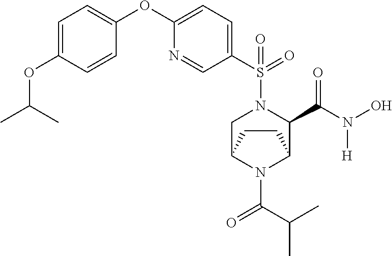 [SMILES: CC(C)Oc1ccc(Oc2ccc(S(=O)(=O)N3C[C@H]4CC[C@@H]([C@@H]3C(=O)NO)N4C(=O)C(C)C)cn2)cc1] | (1S,2R,5R)-N-hydroxy-8-isobutyryl-3-((6- (4-isopropoxyphenoxy)pyridin-3- yl)sulfonyl)-3,8- diazabicyclo[3.2.1]octane-2-carboxamide |
| 61 | 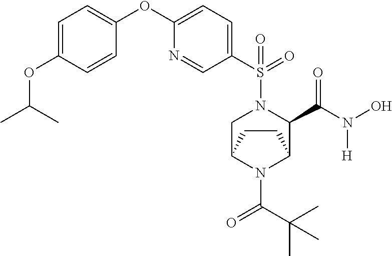 [SMILES: CC(C)Oc1ccc(Oc2ccc(S(=O)(=O)N3C[C@H]4CC[C@@H]([C@@H]3C(=O)NO)N4C(=O)C(C)(C)C)cn2)cc1] | (1S,2R,5R)-N-hydroxy-3-((6-(4- isopropoxyphenoxy)pyridin-3- yl)sulfonyl)-8-pivaloyl-3,8- diazabicyclo[3.2.1]octane-2-carboxamide |
| 62 | 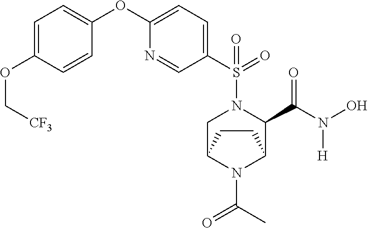 [SMILES: CC(=O)N1[C@@H]2CC[C@H]1[C@H](C(=O)NO)N(S(=O)(=O)c1ccc(Oc3ccc(OCC(F)(F)F)cc3)nc1)C2] | (1S,2R,5R)-8-acetyl-N-hydroxy-3-((6-(4- (2,2,2-trifluoroethoxy)phenoxy)pyridin-3- yl)sulfonyl)-3,8- diazabicyclo[3.2.1]octane-2-carboxamide |
| 63 | 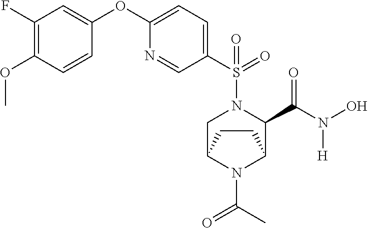 [SMILES: COc1ccc(Oc2ccc(S(=O)(=O)N3C[C@H]4CC[C@@H]([C@@H]3C(=O)NO)N4C(C)=O)cn2)cc1F] | (1S,2R,5R)-8-acetyl-3-((6-(3-fluoro-4- methoxyphenoxy)pyridin-3-yl)sulfonyl)- N-hydroxy-3,8-diazabicyclo[3.2.1]octane- 2-carboxamide |
| 64 | 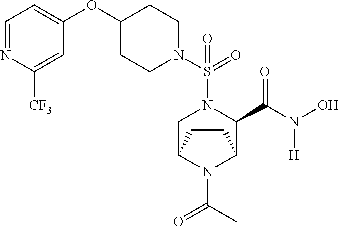 [SMILES: CC(=O)N1[C@@H]2CC[C@H]1[C@H](C(=O)NO)N(S(=O)(=O)N1CCC(Oc3ccnc(C(F)(F)F)c3)CC1)C2] | (1S,2R,5R)-8-acetyl-N-hydroxy-3-((4-((2- (trifluoromethyl)pyridin-4- yl)oxy)piperidin-1-yl)sulfonyl)-3,8- diazabicyclo[3.2.1]octane-2-carboxamide |
| 65 | 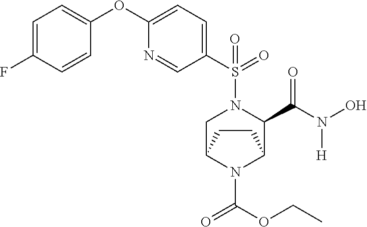 [SMILES: CCOC(=O)N1[C@@H]2CC[C@H]1[C@H](C(=O)NO)N(S(=O)(=O)c1ccc(Oc3ccc(F)cc3)nc1)C2] | ethyl (1S,2R,5R)-3-((6-(4- fluorophenoxy)pyridin-3-yl)sulfonyl)-2- (hydroxycarbamoyl)-3,8- diazabicyclo[3.2.1]octane-8-carboxylate |
| 66 | 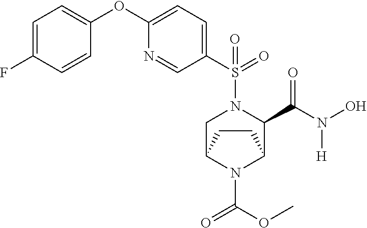 [SMILES: COC(=O)N1[C@@H]2CC[C@H]1[C@H](C(=O)NO)N(S(=O)(=O)c1ccc(Oc3ccc(F)cc3)nc1)C2] | methyl (1S,2R,5R)-3-((6-(4- fluorophenoxy)pyridin-3-yl)sulfonyl)-2- (hydroxycarbamoyl)-3,8- diazabicyclo[3.2.1]octane-8-carboxylate |
| 67 | 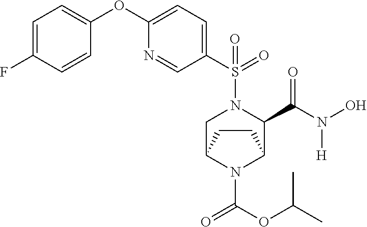 [SMILES: CC(C)OC(=O)N1[C@@H]2CC[C@H]1[C@H](C(=O)NO)N(S(=O)(=O)c1ccc(Oc3ccc(F)cc3)nc1)C2] | isopropyl (1S,2R,5R)-3-((6-(4- fluorophenoxy)pyridin-3-yl)sulfonyl)-2- (hydroxycarbamoyl)-3,8- diazabicyclo[3.2.1]octane-8-carboxylate |
| 68 | 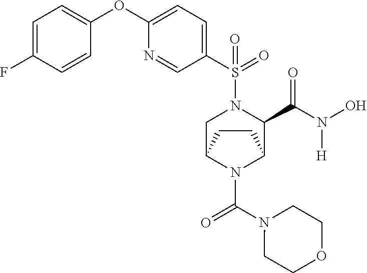 [SMILES: O=C(NO)[C@H]1[C@@H]2CC[C@H](CN1S(=O)(=O)c1ccc(Oc3ccc(F)cc3)nc1)N2C(=O)N1CCOCC1] | (1S,2R,5R)-3-((6-(4- fluorophenoxy)pyridin-3-yl)sulfonyl)-N- hydroxy-8-(morpholine-4-carbonyl)-3,8- diazabicyclo[3.2.1]octane-2-carboxamide |
| 69 | 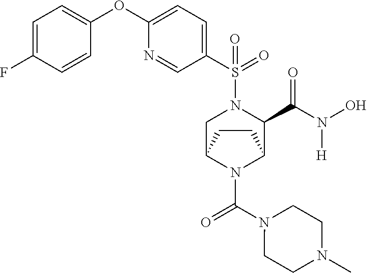 [SMILES: CN1CCN(C(=O)N2[C@@H]3CC[C@H]2[C@H](C(=O)NO)N(S(=O)(=O)c2ccc(Oc4ccc(F)cc4)nc2)C3)CC1] | (1S,2R,5R)-3-((6-(4- fluorophenoxy)pyridin-3-yl)sulfonyl)-N- hydroxy-8-(4-methylpiperazine-1- carbonyl)-3,8-diazabicyclo[3.2.1]octane-2- carboxamide |
| 70 | 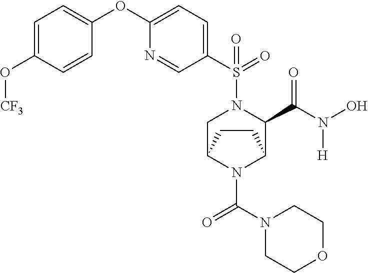 [SMILES: O=C(NO)[C@H]1[C@@H]2CC[C@H](CN1S(=O)(=O)c1ccc(Oc3ccc(OC(F)(F)F)cc3)nc1)N2C(=O)N1CCOCC1] | (1S,2R,5R)-N-hydroxy-8-(morpholine-4- carbonyl)-3-((6-(4- (trifluoromethoxy)phenoxy)pyridin-3- yl)sulfonyl)-3,8- diazabicyclo[3.2.1]octane-2-carboxamide |
| 71 | 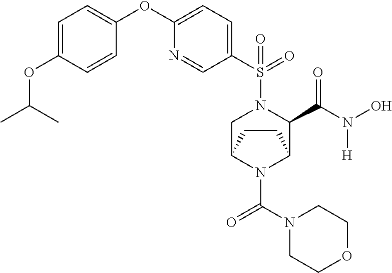 [SMILES: CC(C)Oc1ccc(Oc2ccc(S(=O)(=O)N3C[C@H]4CC[C@@H]([C@@H]3C(=O)NO)N4C(=O)N3CCOCC3)cn2)cc1] | (1S,2R,5R)-N-hydroxy-3-((6-(4- isopropoxyphenoxy)pyridin-3- yl)sulfonyl)-8-(morpholine-4-carbonyl)- 3,8-diazabicyclo[3.2.1]octane-2- carboxamide |
| 72 | 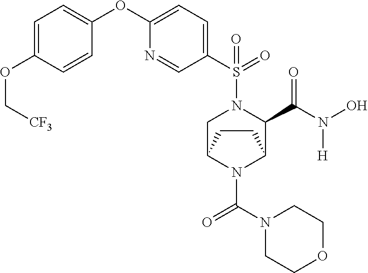 [SMILES: O=C(NO)[C@H]1[C@@H]2CC[C@H](CN1S(=O)(=O)c1ccc(Oc3ccc(OCC(F)(F)F)cc3)nc1)N2C(=O)N1CCOCC1] | (1S,2R,5R)-N-hydroxy-8-(morpholine-4- carbonyl)-3-((6-(4-(2,2,2- trifluoroethoxy)phenoxy)pyridin-3- yl)sulfonyl)-3,8- diazabicyclo[3.2.1]octane-2-carboxamide |
| 73 | 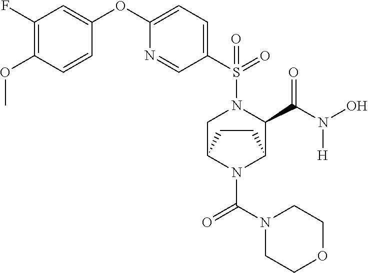 [SMILES: COc1ccc(Oc2ccc(S(=O)(=O)N3C[C@H]4CC[C@@H]([C@@H]3C(=O)NO)N4C(=O)N3CCOCC3)cn2)cc1F] | (1S,2R,5R)-3-((6-(3-fluoro-4- methoxyphenoxy)pyridin-3-yl)sulfonyl)- N-hydroxy-8-(morpholine-4-carbonyl)- 3,8-diazabicyclo[3.2.1]octane-2- carboxamide |
| 74 | 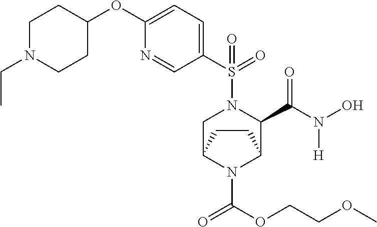 [SMILES: CCN1CCC(Oc2ccc(S(=O)(=O)N3C[C@H]4CC[C@@H]([C@@H]3C(=O)NO)N4C(=O)OCCOC)cn2)CC1] | 2-methoxyethyl (1S,2R,5R)-3-((6-((1- ethylpiperidin-4-yl)oxy)pyridin-3- yl)sulfonyl)-2-(hydroxycarbamoyl)-3,8- diazabicyclo[3.2.1]octane-8-carboxylate |
| 75 | 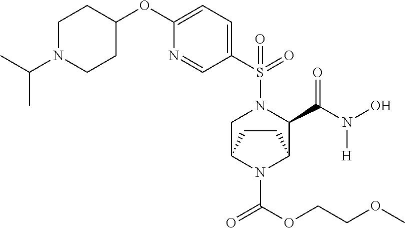 [SMILES: COCCOC(=O)N1[C@@H]2CC[C@H]1[C@H](C(=O)NO)N(S(=O)(=O)c1ccc(OC3CCN(C(C)C)CC3)nc1)C2] | 2-methoxyethyl (1S,2R,5R)-2- (hydroxycarbamoyl)-3-((6-((1- isopropylpiperidin-4-yl)oxy)pyridin-3- yl)sulfonyl)-3,8- diazabicyclo[3.2.1]octane-8-carboxylate |
| 76 | 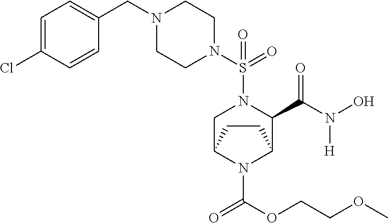 [SMILES: COCCOC(=O)N1[C@@H]2CC[C@H]1[C@H](C(=O)NO)N(S(=O)(=O)N1CCN(Cc3ccc(Cl)cc3)CC1)C2] | 2-methoxyethyl (1S,2R,5R)-3-((4-(4- chlorobenzyl)piperazin-1-yl)sulfonyl)-2- (hydroxycarbamoyl)-3,8- diazabicyclo[3.2.1]octane-8-carboxylate |
| 77 | 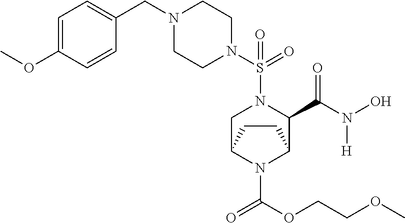 | 2-methoxyethyl (1S,2R,5R)-2- (hydroxycarbamoyl)-3-((4-(4- methoxybenzyl)piperazin-1-yl)sulfonyl)- 3,8-diazabicyclo[3.2.1]octane-8- carboxylate |
| 78 | 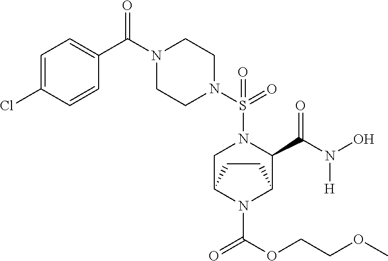 [SMILES: COCCOC(=O)N1[C@@H]2CC[C@H]1[C@H](C(=O)NO)N(S(=O)(=O)N1CCN(C(=O)c3ccc(Cl)cc3)CC1)C2] | 2-methoxyethyl (1S,2R,5R)-3-((4-(4- chlorobenzoyl)piperazin-1-yl)sulfonyl)-2- (hydroxycarbamoyl)-3,8- diazabicyclo[3.2.1]octane-8-carboxylate |
| 79 | 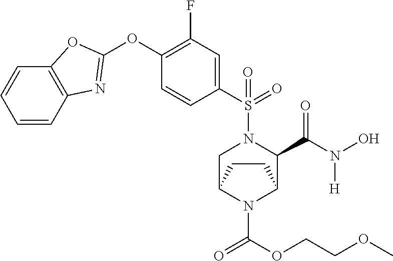 [SMILES: COCCOC(=O)N1[C@@H]2CC[C@H]1[C@H](C(=O)NO)N(S(=O)(=O)c1ccc(Oc3nc4ccccc4o3)c(F)c1)C2] | 2-methoxyethyl (1S,2R,5R)-3-((4- (benzo[d]oxazol-2-yloxy)-3- fluorophenyl)sulfonyl)-2- (hydroxycarbamoyl)-3,8- diazabicyclo[3.2.1]octane-8-carboxylate |
| 80 | 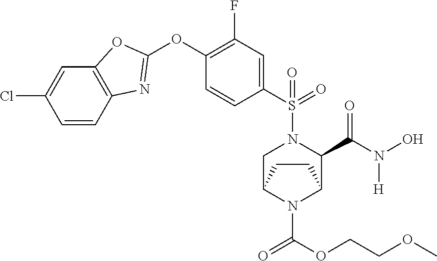 [SMILES: COCCOC(=O)N1[C@@H]2CC[C@H]1[C@H](C(=O)NO)N(S(=O)(=O)c1ccc(Oc3nc4ccc(Cl)cc4o3)c(F)c1)C2] | 2-methoxyethyl (1S,2R,5R)-3-((4-((6- chlorobenzo[d]oxazol-2-yl)oxy)-3- fluorophenyl)sulfonyl)-2- (hydroxycarbamoyl)-3,8- diazabicyclo[3.2.1]octane-8-carboxylate |
| 81 | 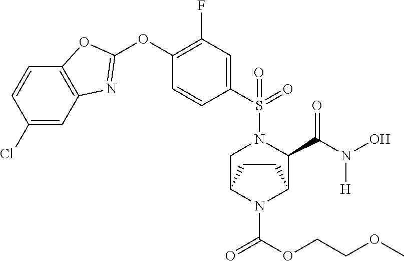 [SMILES: COCCOC(=O)N1[C@@H]2CC[C@H]1[C@H](C(=O)NO)N(S(=O)(=O)c1ccc(Oc3nc4cc(Cl)ccc4o3)c(F)c1)C2] | 2-methoxyethyl (1S,2R,5R)-3-((4-((5- chlorobenzo[d]oxazol-2-yl)oxy)-3- fluorophenyl)sulfonyl)-2- (hydroxycarbamoyl)-3,8- diazabicyclo[3.2.1]octane-8-carboxylate |
| 82 | 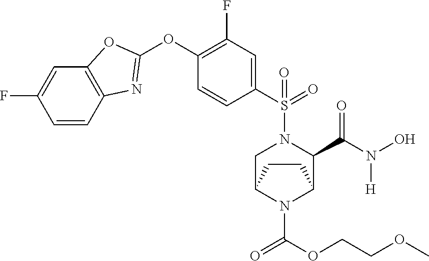 [SMILES: COCCOC(=O)N1[C@@H]2CC[C@H]1[C@H](C(=O)NO)N(S(=O)(=O)c1ccc(Oc3nc4ccc(F)cc4o3)c(F)c1)C2] | 2-methoxyethyl (1S,2R,5R)-3-((3-fluoro- 4-((6-fluorobenzo[d]oxazol-2- yl)oxy)phenyl)sulfonyl)-2- (hydroxycarbamoyl)-3,8- diazabicyclo[3.2.1]octane-8-carboxylate |
| 83 | 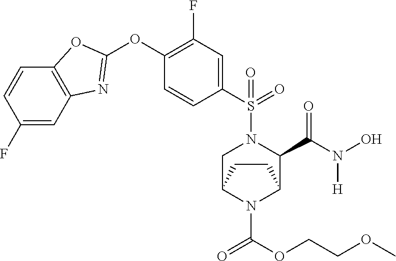 [SMILES: COCCOC(=O)N1[C@@H]2CC[C@H]1[C@H](C(=O)NO)N(S(=O)(=O)c1ccc(Oc3nc4cc(F)ccc4o3)c(F)c1)C2] | 2-methoxyethyl (1S,2R,5R)-3-((3-fluoro- 4-((5-fluorobenzo[d]oxazol-2- yl)oxy)phenyl)sulfonyl)-2- (hydroxycarbamoyl)-3,8- diazabicyclo[3.2.1]octane-8-carboxylate |
| 84 | 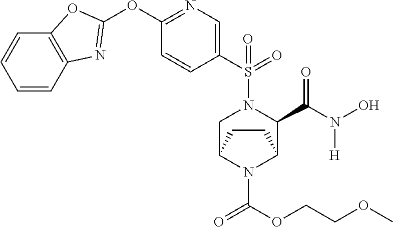 [SMILES: COCCOC(=O)N1[C@@H]2CC[C@H]1[C@H](C(=O)NO)N(S(=O)(=O)c1ccc(Oc3nc4ccccc4o3)nc1)C2] | 2-methoxyethyl (1S,2R,5R)-3-((6- (benzo[d]oxazol-2-yloxy)pyridin-3- yl)sulfonyl)-2-(hydroxycarbamoyl)-3,8- diazabicyclo[3.2.1]octane-8-carboxylate |

or is a pharmaceutically acceptable salt or solvate thereof.

In another aspect disclosed herein is a pharmaceutical composition comprising a pharmaceutically acceptable diluent, excipient, or binder, and a compound of Formula (I) or (1a) or (Ib) or (Ic) or (II) or (III) or (IIIa) or (IIIb) or (IIIc), or a pharmaceutically acceptable salt or solvate thereof.

In another aspect disclosed herein is a method for treating a disease, disorder or condition selected from:
cardiovascular disease, heart failure, congestive heart failure, heart failure with reduced ejection fraction, heart failure with reserved ejection fraction, impaired cardiac contractility, age-related cardiac hypertrophy, inflammation and fibrosis, viral myocarditis, COVID-19 myocarditis, COVID-19 related myocardial fibrosis, pressure overload hypertrophy, myocardial fibrosis, myocardial infarction, myocardial ischemia/reperfusion injury, pathological remodeling of myocardium, ECM remodeling following myocardial injury, radiation myocarditis, radiation myocardial fibrosis, chemotherapy cardiomyopathy, vessel rarefaction, aortic valve sclerosis, calcific aortic valve stenosis, aortic aneurism, abdominal aorta aneurism, giant cell arteritis, age-associated arterial fibrosis, pulmonary hypertension, right ventricle hypertrophy,
idiopathic pulmonary fibrosis, acute lung injury (ALI), acute respiratory distress syndrome (ARDS), Hermansky-Pudlak syndrome (HPS), chronic obstructive pulmonary disease (COPD), emphysema,
polycystic kidney disease, membranous nephropathy, diabetic nephropathy, acute kidney injury, glomerulonephritis, inherited kidney disease, and chronic allograft nephropathy, focal segmental glomerulosclerosis, minimal change disease, human immunodeficiency virus-associated nephropathy, anti-neutrophil cytoplasmic antibody-associated vasculitis, lupus nephritis, IgA nephropathy, Henoch-Schoenlein purpura, and postinfectious glomerulonephritis, membranoproliferative glomerulonephritis, cisplatin-induced renal injury, tubular injury following sepsis], acute ischemic kidney injury, contrast-induced kidney injury, acute tubular injury after ischemia and reperfusion, end-stage renal disease, tubulointerstitial fibrosis, alcoholic liver disease, non-alcoholic fatty liver disease, non-alcoholic steatohepatitis, steatosis, cirrhosis, hepatic ischemia and reperfusion injury, viral hepatitis, drug-induced liver injury, primary biliary cholangitis, primary sclerosing cholangitis, hemochromatosis, Wilson's disease, acute liver failure, biliary atresia,
and scleroderma pigmentosum,
in a mammal in need thereof, comprising administering to the mammal in need thereof a therapeutically effective amount of a compound of Formula (I) or (Ia) or (Ib) or (Ic) or (II) or (III) or (IIIa) or (IIIb) or (IIIc), or a pharmaceutically acceptable salt or solvate thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range varies between 1% and 15% of the stated number or numerical range. The term “comprising” (and related terms such as “comprise” or “comprises” or “having” or “including”) is not intended to exclude that which in other certain embodiments, for example, an embodiment of any composition of matter, composition, method, or process, or the like, described herein, may “consist of” or “consist essentially of” the described features.

### Definitions

As used in the specification and appended claims, unless specified to the contrary, the following terms have the meaning indicated below.

The terms used herein may be preceded and/or followed by a single dash “—”, or a double dash “═”, to indicate the bond order of the bond between the named substituent and its parent moiety; a single dash indicates a single bond and a double dash indicates a double bond. In the absence of a single or double dash, it is understood that a single bond is formed between the substituent and its parent moiety; further, substituents are intended to be read “from left to right,” unless a dash indicates otherwise. For example, C₁-C₆alkoxycarbonyloxy and —OC(O)O—C₁-C₆alkyl indicate the same functionality; similarly arylalkyl and -alkylaryl indicate the same functionality.

For purposes of the invention, the chemical elements are identified in accordance with the Periodic Table of the Elements, CAS version, Handbook of Chemistry and Physics, 67th ed., 1986-87, inside cover.

As used herein, C₁-Cₓ includes C₁-C₂, C₁-C₃ . . . C₁-Cₓ. C₁-Cₓ refers to the number of carbon atoms that make up the moiety to which it designates (excluding optional substituents).

The terms “hydrogen”, “hydrogen atom”, and symbol “H”, as used herein in the context of substituents to structural formulas, such as Formula (I) or (Ia) or (Ib) or (Ic) or (II) or (III) or (IIIa) or (IIIb) or (IIIc), denote a hydrogen atom attached to the remaining part of the molecule or group in question. For the sake of simplicity, hydrogen atoms attached to carbon atoms are not shown in the structural formulas; each carbon atom is understood to be associated with enough hydrogen atoms to give the carbon atom four bonds.

A “saturated” or “fully saturated” compound means that the referenced chemical structure does not contain any multiple carbon-carbon bonds. For example, a saturated cycloalkyl group as defined herein includes cyclohexyl, cyclopropyl, and the like.

An “unsaturated” or “partially saturated” compound means that the referenced chemical structure may contains on or more multiple carbon-carbon bonds, but is not aromatic. For example, an unsaturated cycloalkyl group as defined herein includes cyclohexenyl, cyclopentenyl, cyclohexadienyl, and the like.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, fragmentation, decomposition, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, halogen, azide, alkyl, aralkyl, alkenyl, alkynyl, cycloalkyl, cycloalkylalkyl, (cycloalkyl)alkoxyl, hydroxyl, alkoxyl, amino, nitro, sulfhydryl, imino, amido, phosphonate, phosphinate, carbonyl, carboxyl, silyl, ether, alkylthio, sulfonyl, aminosulfonyl, sulfonamido, ketone, aldehyde, ester, heterocyclyl, heterocyclylalkyl, aromatic or heteroaromatic moieties, aminoalkyl, haloalkyl, fluoroalkyl (such as trifluoromethyl), haloalkoxyl, cyano, or other substituents described above. The permissible substituents may be one or more and the same or different for appropriate organic compounds.

For purposes of this specification, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

“Alkyl” or “alkylene” refers to a straight or branched hydrocarbon chain radical consisting solely of carbon and hydrogen atoms, containing no unsaturation, having from one to fifteen carbon atoms (e.g., —C₁-C₁₅ alkyl). In certain embodiments, an alkyl comprises one to thirteen carbon atoms (e.g., —C₁-C₁₃ alkyl). In certain embodiments, an alkyl comprises one to eight carbon atoms (e.g., —C₁-C₈ alkyl). In other embodiments, an alkyl comprises one to six carbon atoms (e.g., —C₁-C₆ alkyl). In other embodiments, an alkyl comprises one to five carbon atoms (e.g., —C₁-C₅ alkyl). In other embodiments, an alkyl comprises one to four carbon atoms (e.g., —C₁-C₄ alkyl). In other embodiments, an alkyl comprises one to three carbon atoms (e.g., —C₁-C₃ alkyl). In other embodiments, an alkyl comprises one to two carbon atoms (e.g., —C₁-C₂ alkyl). In other embodiments, an alkyl comprises one carbon atom (e.g., —C₁ alkyl). In other embodiments, an alkyl comprises five to fifteen carbon atoms (e.g., —C₅-C₁₅ alkyl). In other embodiments, an alkyl comprises five to eight carbon atoms (e.g., —C₅-C₈ alkyl). In other embodiments, an alkyl comprises two to five carbon atoms (e.g., —C₂-C₅ alkyl). In other embodiments, an alkyl comprises three to five carbon atoms (e.g., —C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl), and 2,2-dimethylpropyl (neopentyl). The alkyl is attached to the rest of the molecule by a single bond. The alkylene is a diradical and is attached to two parts of the molecule by two single bonds, such as in the case of methylene (—CH₂—) or ethylene (—CH₂CH₂— or —CH(—CH₃)—). Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, aryl, aralkyl, heterocycloalkyl, heteroaryl or heteroarylalkyl, and each R^{f} is independently alkyl, fluoroalkyl, cycloalkyl, aryl, aralkyl, heterocycloalkyl, heteroaryl or heteroarylalkyl.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{f}, —N(R^{a})₂, —C(O)R^{a}, —C(O)OR^{a}, —C(O)N(R^{a})₂, —N(R^{a})C(O) OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, aryl, aralkyl, heterocycloalkyl, heteroaryl or heteroarylalkyl, and each R^{f} is independently alkyl, fluoroalkyl, cycloalkyl, aryl, aralkyl, heterocycloalkyl, heteroaryl or heteroarylalkyl.

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, aryl, aralkyl, heterocycloalkyl, heteroaryl or heteroarylalkyl, and each R^{f} is independently alkyl, fluoroalkyl, cycloalkyl, aryl, aralkyl, heterocycloalkyl, heteroaryl or heteroarylalkyl.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hückel theory. The ring systems from which aryl groups are derived, include, but are not limited to, cyclic systems such as benzene, fluorene, indane, indene, tetralin and naphthalene, corresponding respectively to phenyl, fluorenyl, indanyl, tetralinyl, and naphthalenyl radicals, that can be further substituted. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, aryl, aralkyl, aralkenyl, aralkynyl, cycloalkyl, heterocycloalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O) OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain. In particular, phenyl can be optionally substituted by one to five substituents independently selected from the above-mentioned substituents. For example, phenyl substituted with halogen represents 2-chlorophenyl, 3-chlorophenyl, 4-chlorophenyl, 2-fluorophenyl, 3-fluorophenyl, 4-fluorophenyl, 2,4,6-trifluorophenyl, 3-chloro-4-fluorophenyl, etc., but also 3-methyl-4-fluorophenyl, and 3,5-dimethyl-4-bromophenyl.

“Aryloxy” refers to a radical bonded through an oxygen atom of the formula —O-aryl, where aryl is as defined above.

“Aralkyl” refers to a radical of the formula —R^{d}-aryl where R^{d} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkyloxy” refers to a radical bonded through an oxygen atom of the formula —O— aralkyl, where aralkyl is as defined above.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a cycloalkyl comprises three to ten carbon atoms. In other embodiments, a cycloalkyl comprises five to seven carbon atoms. The cycloalkyl is attached to the rest of the molecule by a single bond. Cycloalkyls are saturated, (i.e., containing single C—C bonds only) or partially unsaturated (i.e., containing one or more double bonds or triple bonds.) Examples of monocyclic cycloalkyls include, e.g., cyclopropyl, cyclobutyl, cyclopentyl, cyclohexyl, cycloheptyl, and cyclooctyl. In certain embodiments, a cycloalkyl comprises three to eight carbon atoms (e.g., —C₃-C₈ cycloalkyl). In other embodiments, a cycloalkyl comprises three to seven carbon atoms (e.g., —C₃-C₇ cycloalkyl). In other embodiments, a cycloalkyl comprises three to six carbon atoms (e.g., —C₃-C₆ cycloalkyl). In other embodiments, a cycloalkyl comprises three to five carbon atoms (e.g., —C₃-C₅ cycloalkyl). In other embodiments, a cycloalkyl comprises three to four carbon atoms (e.g., —C₃-C₄ cycloalkyl). A partially unsaturated cycloalkyl is also referred to as “cycloalkenyl.” Examples of monocyclic cycloalkenyls include, e.g., cyclopentenyl, cyclohexenyl, cycloheptenyl, and cyclooctenyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “cycloalkyl” is meant to include cycloalkyl radicals optionally substituted by one or more substituents independently selected from alkyl, alkenyl, alkynyl, halo, fluoroalkyl, cyano, nitro, aryl, aralkyl, aralkenyl, aralkynyl, cycloalkyl, heterocycloalkyl, heteroaryl,

heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

“Halo” or “halogen” refers to bromo, chloro, fluoro or iodo substituents.

“Haloalkyl” refers to an alkyl radical, as defined above, that is substituted by one or more halo radicals, as defined above. Examples of haloalkyl include chloromethyl, trichloromethyl, 2,2,2-trifluoroethyl, and 1-bromo-2-chloro-3-fluoropropyl.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. The alkyl parts of the fluoroalkyl radical are optionally substituted as defined above for an alkyl group.

“Haloalkoxy” refers to an alkoxy radical, as defined above, that is substituted by one or more halo radicals, as defined above.

“Heterocycloalkyl” refers to a stable 3- to 18-membered non-aromatic ring system radical that comprises two to twelve carbon atoms and from one to six heteroatoms selected from nitrogen, oxygen and sulfur. “C₂-C₉heterocycloalkyl” refers to a heterocycloalkyl radical as defined above that comprises from two to nine carbon atoms and from one to four heteroatoms. “C₄-C₅heterocycloalkyl” refers to a heterocycloalkyl radical as defined above that comprises from four to five carbon atoms and from one to three heteroatoms. Unless stated otherwise specifically in the specification, the heterocycloalkyl radical is a monocyclic, bicyclic, tricyclic or tetracyclic ring system, which include fused, spiro, or bridged ring systems. The heteroatoms in the heterocycloalkyl radical are optionally oxidized. One or more nitrogen atoms, if present, are optionally quaternized. The heterocycloalkyl radical is partially or fully saturated. In some embodiments, the heterocycloalkyl is attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl (in particular, morpholin-4-yl), octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocycloalkyl” is meant to include heterocycloalkyl radicals as defined above that are optionally substituted by one or more substituents selected from alkyl, alkenyl, alkynyl, halo, fluoroalkyl, oxo, thioxo, cyano, nitro, aryl, aralkyl, aralkenyl, aralkynyl, cycloalkyl, heterocycloalkyl, heteroaryl,

heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{b}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

“Heteroaryl” refers to a radical derived from a 5- to 18-membered aromatic ring system that comprises one to seventeen carbon atoms and from one to six heteroatoms selected from nitrogen, oxygen and sulfur. “C₂-C₉ heteroaryl” refers to a radical derived from a 3- to 10-membered aromatic ring system that comprises two to nine carbon atoms and from one to 4 heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl radicals are exemplified by pyridinyl, quinolinyl, oxazolyl, and benzoxazolyl (i.e., benzo[d]oxazolyl). Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Unless stated otherwise specifically in the specification, the term “heteroaryl” is meant to include heteroaryl radicals as defined above that are optionally substituted by one or more substituents selected from alkyl, alkenyl, alkynyl, halo, haloalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

“N-Heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-Heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroaryloxy” refers to radical bonded through an oxygen atom of the formula —O— heteroaryl, where heteroaryl is as defined above.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

In some embodiments, the compounds disclosed herein contain one or more asymmetric centers and thus give rise to enantiomers, diastereomers, and other stereoisomeric forms that are defined, in terms of absolute stereochemistry, as (R)- or (S)—. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. In certain embodiments, the compounds presented herein exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

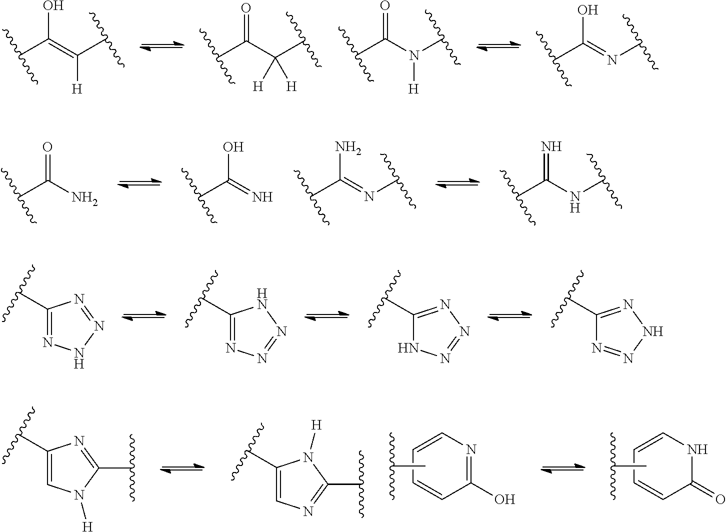

“Optional” or “optionally” means that a subsequently described event or circumstance may or may not occur and that the description includes instances when the event or circumstance occurs and instances in which it does not. For example, “optionally substituted aryl” means that the aryl radical may or may not be substituted and that the description includes both substituted aryl radicals and aryl radicals having no substitution.

The term “prodrugs” includes compounds that, after administration, are metabolized into a pharmacologically active drug (R. B. Silverman, 1992, “The Organic Chemistry of Drug Design and Drug Action,” Academic Press, Chp. 8). A prodrug may be used to improve how a compound is absorbed, distributed, metabolized, and excreted.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“IC₅₀” refers to an amount, concentration or dosage of a particular test compound that achieves a 50% inhibition of a maximal response in an assay that measures such response.

As used herein, “subject” refers to a warm blooded animal such as a mammal, preferably a human, or a human child, which is afflicted with, or has the potential to be afflicted with one or more diseases and disorders described herein.

The term “mammal” refers to a human, a non-human primate, canine, feline, bovine, ovine, porcine, murine, or other veterinary or laboratory mammal. Those skilled in the art recognize that a therapy which reduces the severity of a pathology in one species of mammal is predictive of the effect of the therapy on another species of mammal.

An “effective amount” refers to any amount that is sufficient to achieve a desired biological effect. Combined with the teachings provided herein, by choosing among the various active compounds and weighing factors such as potency, relative bioavailability, patient body weight, sex, age, medical history, severity of adverse side-effects and preferred mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. It is preferred generally that a maximum dose be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

As used herein, “treatment” or “treating” or “palliating” or “ameliorating” are used interchangeably herein. These terms refers to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has not been made.

### Compounds

The compounds of Formula (I) or (Ia) or (Ib) or (Ic) or (II) or (III) or (IIIa) or (IIIb) or (IIIc) described herein are MMP inhibitors. In some embodiments, the compounds of Formula (I) or (Ia) or (Ib) or (Ic) or (II) or (III) or (IIIa) or (IIIb) or (IIIc) described herein, and compositions comprising these compounds, are useful for treating a cardiovascular disease, disorder or condition, in particular heart failure.

In some embodiments, the invention provides a compound of Formula (I):

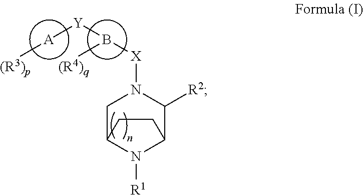

wherein:
\textcircled{A} is phenyl, C₃-C₁₀cycloalkyl, C₂-C₉heterocycloalkyl, or C₂-C₉heteroaryl;
\textcircled{B} is phenyl, C₃-C₁₀cycloalkyl, C₂-C₉heterocycloalkyl, or C₂-C₉heteroaryl;
X is —C(═O)— or —S(═O)₂—;
Y is a direct bond, —O—, —CH₂O—, —OCH₂—, —CH₂—, —C(═O)—, —C(═O)NH—, or —N(⁵)—;
wherein Y, if asymmetrical, is written in the sequence corresponding to

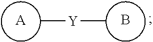

wherein if nitrogen is the attachment point on \textcircled{B} for attaching Y then Y cannot be neither —O—, —CH₂O—, —C(═O)NH—, nor —N(R⁵)—;
R¹ is hydrogen, —C₁-C₆alkyl, —C₁-C₆haloalkyl, —C₁-C₆alkylene-OR⁶, —C₁-C₆alkylene-N(R⁶)₂, —C₁-C₆alkylene-C(═O)—N(R⁶)₂, —C₁-C₆alkylene-C(═O)—C₄-C₅heterocycloalkyl, —C(═O)R⁷, —C(═O)OR⁶, —C(═O)N(R⁶)₂, —S(═O)₂R⁷, or —S(═O)₂N(R⁶)₂;
R² is —C(═O)OH, —C(═O)NH(OH), —CH₂N(OH)C(═O)R⁸, —CH(OH)N(═O), —C(═O)CF₃, —CH₂NHS(═O)₂R⁸, —CH(NH₂)C(═O)OH,

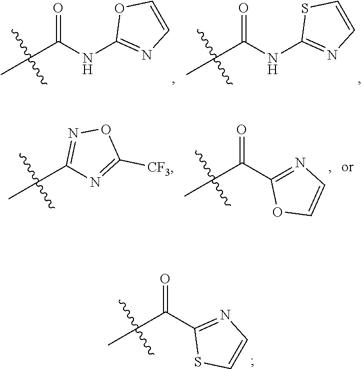 [SMILES: *C(=O)Nc1ncco1 ; *C(=O)Nc1nccs1 ; *c1noc(C(F)(F)F)n1 ; *C(=O)c1ncco1 ; *C(=O)c1nccs1]

each R³ and each R⁴ are independently selected from halogen, —C₁-C₆alkyl, —C₁-C₆haloalkyl, —C₁-C₆alkylene-OR⁶, C₁-C₆haloalkoxy, —OR⁶, —N(R⁶)₂, —CN, —C(═O)R⁷, —C(═O)OR⁶, —C(═O)N(R⁶)₂, —NR⁶C(═O)R⁷, —NR⁶SO₂R⁷, —SO₂R⁷, —SO₂N(R⁶)₂, or phenyl optionally substituted with halogen;
R⁵ is hydrogen or —C₁-C₆alkyl;
each R⁶ is independently selected from hydrogen, —C₁-C₆alkyl, —C₁-C₆haloalkyl, —C₁-C₆alkylene-OR⁹, —C₁-C₆alkylene-N(R⁹)₂, and —C₁-C₆alkylene-C₂-C₉heterocycloalkyl;
or —N(R⁶)₂ represents C₃-C₅heterocycloalkyl;
each R⁷ is independently selected from —C₁-C₆alkyl, —C₁-C₆alkylene-OR⁹, —C₁-C₆alkylene-N(R⁹)₂, —C₂-C₉heterocycloalkyl, —C₂-C₉heteroaryl, —C₁-C₆alkylene-C₂-C₉heterocycloalkyl, and —C₁-C₆alkylene-C₂-C₉heteroaryl;
R⁸ is selected from hydrogen, —C₁-C₆alkyl, and —N(H)C₁-C₆alkyl;
each R⁹ is independently selected from hydrogen and —C₁-C₆alkyl;
each occurrence of C₂-C₉heterocycloalkyl and C₂-C₉heteroaryl being optionally substituted with —C₁-C₆alkyl or —C₁-C₆haloalkyl or halogen;
n is 0, 1, or 2;
p is 0, 1, 2, or 3; and
q is 0, 1, 2, or 3;
   or a pharmaceutically acceptable salt or solvate thereof.

In some embodiments, the invention provides a compound of Formula (I), or a pharmaceutically acceptable salt or solvate thereof, wherein \textcircled{A} is phenyl. In some embodiments, the invention provides a compound of Formula (I), or a pharmaceutically acceptable salt or solvate thereof, wherein \textcircled{A} is C₃-C₁₀cycloalkyl or C₂-C₉heteroaryl. In some embodiments, the invention provides a compound of Formula some embodiments is a compound of Formula (I), or a pharmaceutically acceptable salt or solvate thereof, wherein \textcircled{A} is C₂-C₉heteroaryl.

In some embodiments, the invention provides a compound of Formula (I), or a pharmaceutically acceptable salt or solvate thereof, wherein \textcircled{B} is phenyl. In some embodiments, the invention provides a compound of Formula (I), or a pharmaceutically acceptable salt or solvate thereof, wherein \textcircled{B} is C₃-C₁₀cycloalkyl or C₂-C₉heteroaryl. In some embodiments, the invention provides a compound of Formula some embodiments is a compound of Formula (I), or a pharmaceutically acceptable salt or solvate thereof, wherein \textcircled{B} is C₂-C₉heteroaryl.

In some embodiments, the invention provides a compound of Formula (I), or a pharmaceutically acceptable salt or solvate thereof, wherein \textcircled{A} is phenyl and \textcircled{B} is phenyl.

In some embodiments, the invention provides a compound of Formula (I), or a pharmaceutically acceptable salt or solvate thereof, wherein X is —C(═O)—. In some embodiments, the invention provides a compound of Formula (I), or a pharmaceutically acceptable salt or solvate thereof, wherein X is —S(═O)₂—.

In some embodiments, the invention provides a compound of Formula (I), or a pharmaceutically acceptable salt or solvate thereof, wherein Y is —O—. In some embodiments, the invention provides a compound of Formula (I), or a pharmaceutically acceptable salt or solvate thereof, wherein Y is —CH₂O—. In some embodiments, the invention provides a compound of Formula (I), or a pharmaceutically acceptable salt or solvate thereof, wherein Y is —OCH₂—. In some embodiments, the invention provides a compound of Formula (I), or a pharmaceutically acceptable salt or solvate thereof, wherein Y is —N(R⁵)—. In some embodiments, the invention provides a compound of Formula (I), or a pharmaceutically acceptable salt or solvate thereof, wherein Y is —N(H)—. In some embodiments, the invention provides a compound of Formula (I), or a pharmaceutically acceptable salt or solvate thereof, wherein Y is —N(R⁵)— and R⁵ is —C₁-C₆alkyl.

In some embodiments, the invention provides a compound of Formula (I), or a pharmaceutically acceptable salt or solvate thereof, wherein p is 0. In some embodiments, the invention provides a compound of Formula (I), or a pharmaceutically acceptable salt or solvate thereof, wherein p is 1. In some embodiments, the invention provides a compound of Formula (I), or a pharmaceutically acceptable salt or solvate thereof, wherein p is 2.

In some embodiments, the invention provides a compound of Formula (I), or a pharmaceutically acceptable salt or solvate thereof, wherein each R³ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂. In some embodiments, the invention provides a compound of Formula (I), or a pharmaceutically acceptable salt or solvate thereof, wherein each R³ is independently selected from halogen, —C₁-C₆alkyl, —C₁-C₆haloalkyl, —OR⁶, and —C(═O)OR⁶. In some embodiments, the invention provides a compound of Formula (I), or a pharmaceutically acceptable salt or solvate thereof, wherein each R³ is independently selected from halogen and —C₁-C₆alkyl. In some embodiments, the invention provides a compound of Formula (I), or a pharmaceutically acceptable salt or solvate thereof, wherein each R³ is halogen. In some embodiments, the invention provides a compound of Formula (I), or a pharmaceutically acceptable salt or solvate thereof, wherein each R³ is —C₁-C₆alkyl. In a further embodiment, R³ is methyl. In a further embodiment, R³ is phenyl optionally substituted with halogen. In some embodiments, the invention provides a compound of Formula (I), or a pharmaceutically acceptable salt or solvate thereof, wherein each R³ is —C₁-C₆haloalkyl. In a further embodiment, R³ is trifluoromethyl. In some embodiments, the invention provides a compound of Formula (I), or a pharmaceutically acceptable salt or solvate thereof, wherein each R³ is —OR⁶. In some embodiments, the invention provides a compound of Formula (I), or a pharmaceutically acceptable salt or solvate thereof, wherein each R³ is —OR⁶ and each R⁶ is —C₁-C₆alkyl. In some embodiments, the invention provides a compound of Formula (I), or a pharmaceutically acceptable salt or solvate thereof, wherein each R³ is —C(═O)OR⁶. In a further embodiment, R⁶ is hydrogen or methyl. In some embodiments, the invention provides a compound of Formula (I), or a pharmaceutically acceptable salt or solvate thereof, wherein each R³ is —OR⁶ and each R⁶ is —C₁-C₆alkyl. In some embodiments, the invention provides a compound of Formula (I), or a pharmaceutically acceptable salt or solvate thereof, wherein each R³ is —N(R⁶)₂. In some embodiments, the invention provides a compound of Formula (I), or a pharmaceutically acceptable salt or solvate thereof, wherein each R³ is —C(═O)N(R⁶)₂. In some embodiments, the invention provides a compound of Formula (I), or a pharmaceutically acceptable salt or solvate thereof, wherein each R³ is —SO₂N(R⁶)₂. In some embodiments, —N(R⁶)₂ represents C₃-C₅heterocycloalkyl, e.g., 4-morpholinyl.

In some embodiments, the invention provides a compound of Formula (I), or a pharmaceutically acceptable salt or solvate thereof, wherein q is 0. In some embodiments, the invention provides a compound of Formula (I), or a pharmaceutically acceptable salt or solvate thereof, wherein q is 1. In some embodiments, the invention provides a compound of Formula (I), or a pharmaceutically acceptable salt or solvate thereof, wherein q is 2.

In some embodiments, the invention provides a compound of Formula (I), or a pharmaceutically acceptable salt or solvate thereof, wherein each R⁴ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂. In some embodiments, the invention provides a compound of Formula (I), or a pharmaceutically acceptable salt or solvate thereof, wherein each R⁴ is independently selected from halogen, —C₁-C₆alkyl, —C₁-C₆haloalkyl, —OR⁶, and —C(═O)OR⁶. In some embodiments, the invention provides a compound of Formula (I), or a pharmaceutically acceptable salt or solvate thereof, wherein each R⁴ is independently selected from halogen and —C₁-C₆alkyl. In some embodiments, the invention provides a compound of Formula (I), or a pharmaceutically acceptable salt or solvate thereof, wherein each R⁴ is halogen. In some embodiments, the invention provides a compound of Formula (I), or a pharmaceutically acceptable salt or solvate thereof, wherein each R⁴ is —C₁-C₆alkyl. In a further embodiment, R⁴ is methyl. In a further embodiment, R⁴ is phenyl optionally substituted with halogen. In some embodiments, the invention provides a compound of Formula (I), or a pharmaceutically acceptable salt or solvate thereof, wherein each R⁴ is —C₁-C₆haloalkyl. In some embodiments, the invention provides a compound of Formula (I), or a pharmaceutically acceptable salt or solvate thereof, wherein each R⁴ is —C₁-C₆haloalkyl. In a further embodiment, R⁴ is trifluoromethyl. In some embodiments, the invention provides a compound of Formula (I), or a pharmaceutically acceptable salt or solvate thereof, wherein each R⁴ is independently selected from —OR⁶, —N(R⁶)₂, —C(═O)OR⁶, —C(═O)N(R⁶)₂, and —SO₂N(R⁶)₂. In some embodiments, the invention provides a compound of Formula (I), or a pharmaceutically acceptable salt or solvate thereof, wherein each R⁴ is —OR⁶. In a further embodiment, R⁶ is hydrogen or methyl. In some embodiments, the invention provides a compound of Formula (I), or a pharmaceutically acceptable salt or solvate thereof, wherein each R⁴ is —OR⁶ and each R⁶ is —C₁-C₆alkyl. In some embodiments, the invention provides a compound of Formula (I), or a pharmaceutically acceptable salt or solvate thereof, wherein each R⁴ is —N(R⁶)₂. In some embodiments, the invention provides a compound of Formula (I), or a pharmaceutically acceptable salt or solvate thereof, wherein each R⁴ is —C(═O)OR⁶. In some embodiments, the invention provides a compound of Formula (I), or a pharmaceutically acceptable salt or solvate thereof, wherein each R⁴ is —C(═O)N(R⁶)₂. In some embodiments, the invention provides a compound of Formula (I), or a pharmaceutically acceptable salt or solvate thereof, wherein each R⁴ is —SO₂N(R⁶)₂. In some embodiments, —N(R⁶)₂ represents C₃-C₅heterocycloalkyl, e.g., 4-morpholinyl.

In some embodiments, the invention provides a compound of Formula (I), or a pharmaceutically acceptable salt or solvate thereof, wherein R¹ is —C₁-C₆alkyl, —C₁-C₆haloalkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (I), or a pharmaceutically acceptable salt or solvate thereof, wherein R¹ is —C₁-C₆alkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (I), or a pharmaceutically acceptable salt or solvate thereof, wherein R¹ is —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (I), or a pharmaceutically acceptable salt or solvate thereof, wherein R¹ is —C(═O)OR⁶. In some embodiments, the invention provides a compound of Formula (I), or a pharmaceutically acceptable salt or solvate thereof, wherein R¹ is —C(═O)OR⁶ and R⁶ is —C₁-C₆alkyl, —C₁-C₆alkylene-OR⁹, or —C₁-C₆alkylene-N(R⁹)₂. In some embodiments, the invention provides a compound of Formula (I), or a pharmaceutically acceptable salt or solvate thereof, wherein R¹ is —C(═O)OR⁶. In some embodiments, the invention provides a compound of Formula (I), or a pharmaceutically acceptable salt or solvate thereof, wherein R¹ is —C(═O)OR⁶ and R⁶ is —C₁-C₆alkyl. In some embodiments, the invention provides a compound of Formula (I), or a pharmaceutically acceptable salt or solvate thereof, wherein R⁶ is methyl, ethyl, n-propyl, isobutyl, or t-butyl. In another embodiment, R⁶ is methyl. In yet a further embodiment, R⁶ is ethyl. In some embodiments, the invention provides a compound of Formula (I), or a pharmaceutically acceptable salt or solvate thereof, wherein R¹ is —C(═O)OR⁶ and R⁶ is —C₁-C₆alkylene-OR⁹. In some embodiments, the invention provides a compound of Formula (I), or a pharmaceutically acceptable salt or solvate thereof, wherein R¹ is —C(═O)OR⁶, R⁶ is —C₁-C₆alkylene-OR⁹, and R⁹ is —C₁-C₆alkyl. In a further embodiment, R⁶ is ethylene-OR⁹. In another embodiment, R⁶ is methylene-OR⁹. In another embodiment, R⁹ is selected from methyl, ethyl, n-propyl, or t-butyl. In one embodiment, R⁹ is methyl. In some embodiments, the invention provides a compound of Formula (I), or a pharmaceutically acceptable salt or solvate thereof, wherein R¹ is —C(═O)OR⁶ and R⁶ is —C₁-C₆alkylene-N(R⁹)₂. In some embodiments, the invention provides a compound of Formula (I), or a pharmaceutically acceptable salt or solvate thereof, wherein R¹ is —C(═O)N(R⁶)₂. In some embodiments, the invention provides a compound of Formula (I), or a pharmaceutically acceptable salt or solvate thereof, wherein R¹ is —C(═O)N(R⁶)₂ and each R⁶ is independently selected from hydrogen and —C₁-C₆alkyl. In some embodiments, the invention provides a compound of Formula (I), or a pharmaceutically acceptable salt or solvate thereof, wherein R¹ is —C(═O)N(R⁶)₂ and each R⁶ is independently selected from hydrogen and —C₁-C₆alkylene-OR⁹. In some embodiments, the invention provides a compound of Formula (I), or a pharmaceutically acceptable salt or solvate thereof, wherein R¹ is —S(═O)₂R⁷. In some embodiments, the invention provides a compound of Formula (I), or a pharmaceutically acceptable salt or solvate thereof, wherein R¹ is —S(═O)₂N(R⁶)₂. In some embodiments, the invention provides a compound of Formula (I), or a pharmaceutically acceptable salt or solvate thereof, wherein R¹ is —C(═O)R⁷.

In some embodiments, the invention provides a compound of Formula (I), or a pharmaceutically acceptable salt or solvate thereof, wherein R² is —C(═O)NH(OH), —CH₂N(OH)C(═O)R⁸, —CH(OH)N(═O), —C(═O)CF₃, —CH₂NHS(═O)₂R⁸, —CH(NH₂)C(═O)OH,

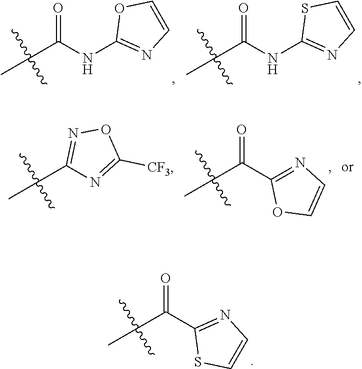 [SMILES: *C(=O)Nc1ncco1 ; *C(=O)Nc1nccs1 ; *c1noc(C(F)(F)F)n1 ; *C(=O)c1ncco1 ; *C(=O)c1nccs1]

In some embodiments, the invention provides a compound of Formula (I), or a pharmaceutically acceptable salt or solvate thereof, wherein R² is —C(═O)NH(OH). In some embodiments, the invention provides a compound of Formula (I), or a pharmaceutically acceptable salt or solvate thereof, wherein R² is —CH₂N(OH)C(═O)R⁸. In some embodiments, the invention provides a compound of Formula (I), or a pharmaceutically acceptable salt or solvate thereof, wherein R² is —CH(OH)N(═O). In some embodiments, the invention provides a compound of Formula (I), or a pharmaceutically acceptable salt or solvate thereof, wherein R² is —C(═O)CF₃. In some embodiments, the invention provides a compound of Formula (I), or a pharmaceutically acceptable salt or solvate thereof, wherein R² is —CH₂NHS(═O)₂R⁸. In some embodiments, the invention provides a compound of Formula (I), or a pharmaceutically acceptable salt or solvate thereof, wherein R² is —CH(NH₂)C(═O)OH. In some embodiments, the invention provides a compound of Formula (I), or a pharmaceutically acceptable salt or solvate thereof, wherein R² is

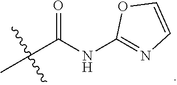 [SMILES: *C(=O)Nc1ncco1]

In some embodiments, the invention provides a compound of Formula (I), or a pharmaceutically acceptable salt or solvate thereof, wherein R² is

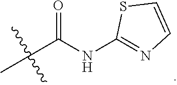 [SMILES: *C(=O)Nc1nccs1]

In some embodiments, the invention provides a compound of Formula (I), or a pharmaceutically acceptable salt or solvate thereof, wherein R² is

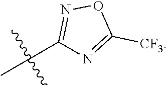 [SMILES: *c1noc(C(F)(F)F)n1]

In some embodiments, the invention provides a compound of Formula (I), or a pharmaceutically acceptable salt or solvate thereof, wherein R² is

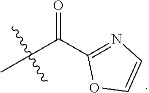 [SMILES: *C(=O)c1ncco1]

In some embodiments, the invention provides a compound of Formula (I), or a pharmaceutically acceptable salt or solvate thereof, wherein R² is

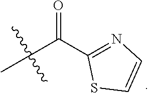 [SMILES: *C(=O)c1nccs1]

In some embodiments, the invention provides a compound of Formula (I), or a pharmaceutically acceptable salt or solvate thereof, wherein n is 1. In some embodiments, the invention provides a compound of Formula (I), or a pharmaceutically acceptable salt or solvate thereof, wherein n is 2. In some embodiments, the invention provides a compound of Formula (I), or a pharmaceutically acceptable salt or solvate thereof, wherein n is 0.

In some embodiments, the invention provides a compound of Formula (Ia):

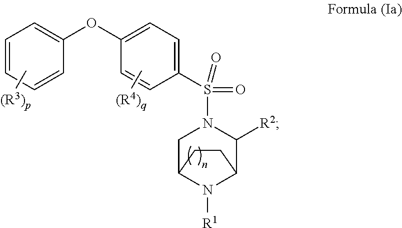

wherein:
R¹ is hydrogen, —C₁-C₆alkyl, —C₁-C₆haloalkyl, —C₁-C₆alkylene-OR⁶, —C₁-C₆alkylene-N(R⁶)₂, —C₁-C₆alkylene-C(═O)—N(R⁶)₂, —C₁-C₆alkylene-C(═O)—C₄-C₅heterocycloalkyl, —C(═O)R⁷, —C(═O)OR⁶, —C(═O)N(R⁶)₂, —S(═O)₂R⁷, or —S(═O)₂N(R⁶)₂;
R² is —C(═O)NH(OH), —CH₂N(OH)C(═O)R⁸, —CH(OH)N(═O), —C(═O)CF₃, —CH₂NHS(═O)₂R⁸, —CH(NH₂)C(═O)OH,

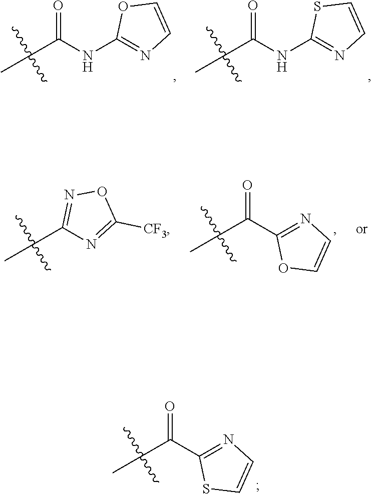 [SMILES: *C(=O)Nc1ncco1 ; *C(=O)Nc1nccs1 ; *c1noc(C(F)(F)F)n1 ; *C(=O)c1ncco1 ; *C(=O)c1nccs1]

each R³ and each R⁴ are independently selected from halogen, —C₁-C₆alkyl, —C₁-C₆haloalkyl, —C₁-C₆alkylene-OR⁶, C₁-C₆haloalkoxy, —OR⁶, —N(R⁶)₂, —CN, —C(═O)R⁷, —C(═O)OR⁶, —C(═O)N(R⁶)₂, —NR⁶C(═O)R⁷, —NR⁶SO₂R⁷, —SO₂R⁷, —SO₂N(R⁶)₂, or phenyl optionally substituted with halogen;
each R⁶ is independently selected from hydrogen, —C₁-C₆alkyl, —C₁-C₆haloalkyl, —C₁-C₆alkylene-OR⁹, —C₁-C₆alkylene-N(R⁹)₂, and —C₁-C₆alkylene-C₂-C₉heterocycloalkyl;
or —N(R⁶)₂ represents C₃-C₅heterocycloalkyl;
each R⁷ is independently selected from —C₁-C₆alkyl, —C₁-C₆alkylene-OR⁹, —C₁-C₆alkylene-N(R⁹)₂, —C₂-C₉heterocycloalkyl, —C₂-C₉heteroaryl, —C₁-C₆alkylene-C₂-C₉heterocycloalkyl, and —C₁-C₆alkylene-C₂-C₉heteroaryl;
R⁸ is selected from hydrogen, —C₁-C₆alkyl, and —N(H)C₁-C₆alkyl;
each R⁹ is independently selected from hydrogen and —C₁-C₆alkyl;
each occurrence of C₂-C₉heterocycloalkyl and C₂-C₉heteroaryl being optionally substituted with —C₁-C₆alkyl or —C₁-C₆haloalkyl or halogen;
n is 0, 1, or 2;
p is 0, 1, 2, or 3; and
q is 0, 1, 2, or 3;
   or a pharmaceutically acceptable salt or solvate thereof.

In some embodiments, the invention provides a compound of Formula (Ia), or a pharmaceutically acceptable salt or solvate thereof, wherein p is 0. In some embodiments, the invention provides a compound of Formula (Ia), or a pharmaceutically acceptable salt or solvate thereof, wherein p is 1. In some embodiments, the invention provides a compound of Formula (Ia), or a pharmaceutically acceptable salt or solvate thereof, wherein p is 2.

In some embodiments, the invention provides a compound of Formula (Ia), or a pharmaceutically acceptable salt or solvate thereof, wherein each R³ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂. In some embodiments, the invention provides a compound of Formula (Ia), or a pharmaceutically acceptable salt or solvate thereof, wherein each R³ is independently selected from halogen, —C₁-C₆alkyl, —C₁-C₆haloalkyl, —OR⁶, and —C(═O)OR⁶. In some embodiments, the invention provides a compound of Formula (Ia), or a pharmaceutically acceptable salt or solvate thereof, wherein each R³ is independently selected from halogen and —C₁-C₆alkyl. In some embodiments, the invention provides a compound of Formula (Ia), or a pharmaceutically acceptable salt or solvate thereof, wherein each R³ is halogen. In some embodiments, the invention provides a compound of Formula (Ia), or a pharmaceutically acceptable salt or solvate thereof, wherein each R³ is —C₁-C₆alkyl. In a further embodiment, R³ is methyl. In some embodiments, the invention provides a compound of Formula (Ia), or a pharmaceutically acceptable salt or solvate thereof, wherein each R³ is —C₁-C₆haloalkyl. In a further embodiment, R³ is trifluoromethyl. In some embodiments, the invention provides a compound of Formula (Ia), or a pharmaceutically acceptable salt or solvate thereof, wherein each R³ is independently selected from —OR⁶, —N(R⁶)₂, —C(═O)OR⁶, —C(═O)N(R⁶)₂, and —SO₂N(R⁶)₂. In some embodiments, the invention provides a compound of Formula (Ia), or a pharmaceutically acceptable salt or solvate thereof, wherein each R³ is —OR⁶. In a further embodiment, R⁶ is hydrogen or methyl. In some embodiments, the invention provides a compound of Formula (Ia), or a pharmaceutically acceptable salt or solvate thereof, wherein each R³ is —OR⁶ and each R⁶ is —C₁-C₆alkyl. In some embodiments, the invention provides a compound of Formula (Ia), or a pharmaceutically acceptable salt or solvate thereof, wherein each R³ is —N(R⁶)₂. In some embodiments, the invention provides a compound of Formula (Ia), or a pharmaceutically acceptable salt or solvate thereof, wherein each R³ is —C(═O)OR⁶. In some embodiments, the invention provides a compound of Formula (Ia), or a pharmaceutically acceptable salt or solvate thereof, wherein each R³ is —C(═O)N(R⁶)₂. In some embodiments, the invention provides a compound of Formula (Ia), or a pharmaceutically acceptable salt or solvate thereof, wherein each R³ is —SO₂N(R⁶)₂.

In some embodiments, the invention provides a compound of Formula (Ia), or a pharmaceutically acceptable salt or solvate thereof, wherein q is 0. In some embodiments, the invention provides a compound of Formula (Ia), or a pharmaceutically acceptable salt or solvate thereof, wherein q is 1. In some embodiments, the invention provides a compound of Formula (Ia), or a pharmaceutically acceptable salt or solvate thereof, wherein q is 2.

In some embodiments, the invention provides a compound of Formula (Ia), or a pharmaceutically acceptable salt or solvate thereof, wherein each R⁴ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂. In some embodiments, the invention provides a compound of Formula (Ia), or a pharmaceutically acceptable salt or solvate thereof, wherein each R⁴ is independently selected from halogen, —C₁-C₆alkyl, —C₁-C₆haloalkyl, —OR⁶, and —C(═O)OR⁶. In some embodiments, the invention provides a compound of Formula (Ia), or a pharmaceutically acceptable salt or solvate thereof, wherein each R⁴ is independently selected from halogen and —C₁-C₆alkyl. In some embodiments, the invention provides a compound of Formula (Ia), or a pharmaceutically acceptable salt or solvate thereof, wherein each R⁴ is halogen. In some embodiments, the invention provides a compound of Formula (Ia), or a pharmaceutically acceptable salt or solvate thereof, wherein each R⁴ is —C₁-C₆alkyl. In a further embodiment, R⁴ is methyl. In some embodiments, the invention provides a compound of Formula (Ia), or a pharmaceutically acceptable salt or solvate thereof, wherein each R⁴ is —C₁-C₆haloalkyl. In a further embodiment, R⁴ is trifluoromethyl. In some embodiments, the invention provides a compound of Formula (Ia), or a pharmaceutically acceptable salt or solvate thereof, wherein each R⁴ is independently selected from —OR⁶, —N(R⁶)₂, —C(═O)OR⁶, —C(═O)N(R⁶)₂, and —SO₂N(R⁶)₂. In some embodiments, the invention provides a compound of Formula (Ia), or a pharmaceutically acceptable salt or solvate thereof, wherein each R⁴ is —OR⁶. In a further embodiment, R⁶ is hydrogen or methyl. In some embodiments, the invention provides a compound of Formula (Ia), or a pharmaceutically acceptable salt or solvate thereof, wherein each R⁴ is —OR⁶ and each R⁶ is —C₁-C₆alkyl. In some embodiments, the invention provides a compound of Formula (Ia), or a pharmaceutically acceptable salt or solvate thereof, wherein each R⁴ is —N(R⁶)₂. In some embodiments, the invention provides a compound of Formula (Ia), or a pharmaceutically acceptable salt or solvate thereof, wherein each R⁴ is —C(═O)OR⁶. In some embodiments, the invention provides a compound of Formula (Ia), or a pharmaceutically acceptable salt or solvate thereof, wherein each R⁴ is —C(═O)N(R⁶)₂. In some embodiments, the invention provides a compound of Formula (Ia), or a pharmaceutically acceptable salt or solvate thereof, wherein each R⁴ is —SO₂N(R⁶)₂.

In some embodiments, the invention provides a compound of Formula (Ta), or a pharmaceutically acceptable salt or solvate thereof, wherein R¹ is —C₁-C₆alkyl, —C₁-C₆haloalkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (Ta), or a pharmaceutically acceptable salt or solvate thereof, wherein R¹ is —C₁-C₆alkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (Ta), or a pharmaceutically acceptable salt or solvate thereof, wherein R¹ is —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (Ia), or a pharmaceutically acceptable salt or solvate thereof, wherein R¹ is —C(═O)OR⁶. In some embodiments, the invention provides a compound of Formula (Ta), or a pharmaceutically acceptable salt or solvate thereof, wherein R¹ is —C(═O)OR⁶ and R⁶ is —C₁-C₆alkyl, —C₁-C₆alkylene-OR⁹, or —C₁-C₆alkylene-N(R⁹)₂. In some embodiments, the invention provides a compound of Formula (Ia), or a pharmaceutically acceptable salt or solvate thereof, wherein R¹ is —C(═O)OR⁶. In some embodiments, the invention provides a compound of Formula (Ia), or a pharmaceutically acceptable salt or solvate thereof, wherein R¹ is —C(═O)OR⁶ and R⁶ is —C₁-C₆alkyl. In some embodiments, the invention provides a compound of Formula (Ta), or a pharmaceutically acceptable salt or solvate thereof, wherein R⁶ is methyl, ethyl, n-propyl, isobutyl, or t-butyl. In another embodiment, R⁶ is methyl. In yet a further embodiment, R⁶ is ethyl. In some embodiments, the invention provides a compound of Formula (Ta), or a pharmaceutically acceptable salt or solvate thereof, wherein R¹ is —C(═O)OR⁶ and R⁶ is —C₁-C₆alkylene-OR⁹. In some embodiments, the invention provides a compound of Formula (Ia), or a pharmaceutically acceptable salt or solvate thereof, wherein R¹ is —C(═O)OR⁶, R⁶ is —C₁-C₆alkylene-OR⁹, and R⁹ is —C₁-C₆alkyl. In a further embodiment, R⁶ is ethylene-OR⁹. In another embodiment, R⁶ is methylene-OR⁹. In another embodiment, R⁹ is selected from methyl, ethyl, n-propyl, or t-butyl. In one embodiment, R⁹ is methyl. In some embodiments, the invention provides a compound of Formula (Ia), or a pharmaceutically acceptable salt or solvate thereof, wherein R¹ is —C(═O)OR⁶ and R⁶ is —C₁-C₆alkylene-N(R⁹)₂. In some embodiments, the invention provides a compound of Formula (Ia), or a pharmaceutically acceptable salt or solvate thereof, wherein R¹ is —C(═O)N(R⁶)₂. In some embodiments, the invention provides a compound of Formula (Ia), or a pharmaceutically acceptable salt or solvate thereof, wherein R¹ is —C(═O)N(R⁶)₂ and each R⁶ is independently selected from hydrogen and —C₁-C₆alkyl. In some embodiments, the invention provides a compound of Formula (Ia), or a pharmaceutically acceptable salt or solvate thereof, wherein R¹ is —C(═O)N(R⁶)₂ and each R⁶ is independently selected from hydrogen and —C₁-C₆alkylene-OR⁹. In some embodiments, the invention provides a compound of Formula (Ia), or a pharmaceutically acceptable salt or solvate thereof, wherein R¹ is —S(═O)₂R⁷. In some embodiments, the invention provides a compound of Formula (Ia), or a pharmaceutically acceptable salt or solvate thereof, wherein R¹ is —S(═O)₂N(R⁶)₂. In some embodiments, the invention provides a compound of Formula (Ia), or a pharmaceutically acceptable salt or solvate thereof, wherein R¹ is —C(═O)R⁷.

In some embodiments, the invention provides a compound of Formula (Ia), or a pharmaceutically acceptable salt or solvate thereof, wherein R² is —C(═O)NH(OH), —CH₂N(OH)C(═O)R⁸, —CH(OH)N(═O), —C(═O)CF₃, —CH₂NHS(═O)₂R⁸, —CH(NH₂)C(═O)OH,

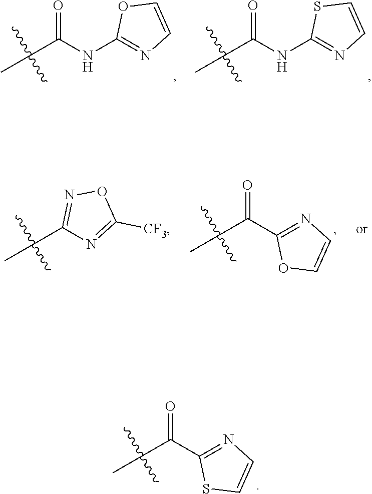 [SMILES: *C(=O)Nc1ncco1 ; *C(=O)Nc1nccs1 ; *c1noc(C(F)(F)F)n1 ; *C(=O)c1ncco1 ; *C(=O)c1nccs1]

In some embodiments, the invention provides a compound of Formula (Ia), or a pharmaceutically acceptable salt or solvate thereof, wherein R² is —C(═O)NH(OH). In some embodiments, the invention provides a compound of Formula (Ia), or a pharmaceutically acceptable salt or solvate thereof, wherein R² is —CH₂N(OH)C(═O)R⁸. In some embodiments, the invention provides a compound of Formula (Ia), or a pharmaceutically acceptable salt or solvate thereof, wherein R² is —CH(OH)N(═O). In some embodiments, the invention provides a compound of Formula (Ia), or a pharmaceutically acceptable salt or solvate thereof, wherein R² is —C(═O)CF₃. In some embodiments, the invention provides a compound of Formula (Ia), or a pharmaceutically acceptable salt or solvate thereof, wherein R² is —CH₂NHS(═O)₂R⁸. In some embodiments, the invention provides a compound of Formula (Ia), or a pharmaceutically acceptable salt or solvate thereof, wherein R² is —CH(NH₂)C(═O)OH. In some embodiments, the invention provides a compound of Formula (Ia), or a pharmaceutically acceptable salt or solvate thereof, wherein R² is

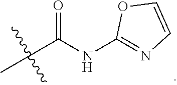 [SMILES: *C(=O)Nc1ncco1]

In some embodiments, the invention provides a compound of Formula (Ia), or a pharmaceutically acceptable salt or solvate thereof, wherein R² is

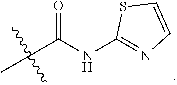 [SMILES: *C(=O)Nc1nccs1]

In some embodiments, the invention provides a compound of Formula (Ia), or a pharmaceutically acceptable salt or solvate thereof, wherein R² is

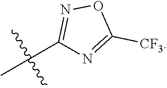 [SMILES: *c1noc(C(F)(F)F)n1]

In some embodiments, the invention provides a compound of Formula (Ia), or a pharmaceutically acceptable salt or solvate thereof, wherein R² is

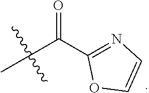 [SMILES: *C(=O)c1ncco1]

In some embodiments, the invention provides a compound of Formula (Ia), or a pharmaceutically acceptable salt or solvate thereof, wherein R² is

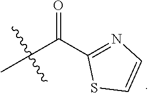 [SMILES: *C(=O)c1nccs1]

In some embodiments, the invention provides a compound of Formula (Ia), or a pharmaceutically acceptable salt or solvate thereof, wherein n is 1. In some embodiments, the invention provides a compound of Formula (Ia), or a pharmaceutically acceptable salt or solvate thereof, wherein n is 2. In some embodiments, the invention provides a compound of Formula (Ia), or a pharmaceutically acceptable salt or solvate thereof, wherein n is 0.

In some embodiments, the invention provides a compound of Formula (I), having the structure of Formula (Ib):

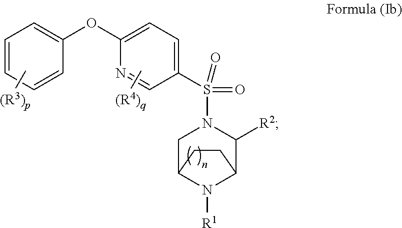

wherein:
R¹ is hydrogen, —C₁-C₆alkyl, —C₁-C₆haloalkyl, —C₁-C₆alkylene-OR⁶, —C₁-C₆alkylene-N(R⁶)₂, —C₁-C₆alkylene-C(═O)—N(R⁶)₂, —C₁-C₆alkylene-C(═O)—C₄-C₅heterocycloalkyl, —C(═O)R⁷, —C(═O)OR⁶, —C(═O)N(R⁶)₂, —S(═O)₂R⁷, or —S(═O)₂N(R⁶)₂;
R² is —C(═O)NH(OH), —CH₂N(OH)C(═O)R⁸, —CH(OH)N(═O), —C(═O)CF₃, —CH₂NHS(═O)₂R⁸, —CH(NH₂)C(═O)OH,

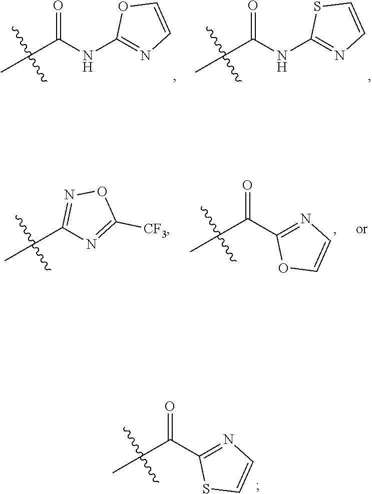 [SMILES: *C(=O)Nc1ncco1 ; *C(=O)Nc1nccs1 ; *c1noc(C(F)(F)F)n1 ; *C(=O)c1ncco1 ; *C(=O)c1nccs1]

each R³ and each R⁴ are independently selected from halogen, —C₁-C₆alkyl, —C₁-C₆haloalkyl, —C₁-C₆alkylene-OR⁶, C₁-C₆haloalkoxy, —OR⁶, —N(R⁶)₂, —CN, —C(═O)R⁷, —C(═O)OR⁶, —C(═O)N(R⁶)₂, —NR⁶C(═O)R⁷, —NR⁶SO₂R⁷, —SO₂R⁷, —SO₂N(R⁶)₂, or phenyl optionally substituted with halogen;
each R⁶ is independently selected from hydrogen, —C₁-C₆alkyl, —C₁-C₆haloalkyl, —C₁-C₆alkylene-OR⁹, —C₁-C₆alkylene-N(R⁹)₂, and —C₁-C₆alkylene-C₂-C₉heterocycloalkyl;
or —N(R⁶)₂ represents C₃-C₅heterocycloalkyl;
each R⁷ is independently selected from —C₁-C₆alkyl, —C₁-C₆alkylene-OR⁹, —C₁-C₆alkylene-N(R⁹)₂, —C₂-C₉heterocycloalkyl, —C₂-C₉heteroaryl, —C₁-C₆alkylene-C₂-C₉heterocycloalkyl, and —C₁-C₆alkylene-C₂-C₉heteroaryl;
R⁸ is selected from hydrogen, —C₁-C₆alkyl, and —N(H)C₁-C₆alkyl;
each R⁹ is independently selected from hydrogen and —C₁-C₆alkyl;
each occurrence of C₂-C₉heterocycloalkyl and C₂-C₉heteroaryl being optionally substituted with —C₁-C₆alkyl or —C₁-C₆haloalkyl or halogen;
n is 0, 1, or 2;
p is 0, 1, 2, or 3; and
q is 0, 1, 2, or 3;
   or a pharmaceutically acceptable salt or solvate thereof.

In some embodiments, the invention provides a compound of Formula (Ib), or a pharmaceutically acceptable salt or solvate thereof, wherein p is 0. In some embodiments, the invention provides a compound of Formula (Ib), or a pharmaceutically acceptable salt or solvate thereof, wherein p is 1. In some embodiments, the invention provides a compound of Formula (Ib), or a pharmaceutically acceptable salt or solvate thereof, wherein p is 2.

In some embodiments, the invention provides a compound of Formula (Ib), or a pharmaceutically acceptable salt or solvate thereof, wherein each R³ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂. In some embodiments, the invention provides a compound of Formula (Ib), or a pharmaceutically acceptable salt or solvate thereof, wherein each R³ is independently selected from halogen, —C₁-C₆alkyl, —C₁-C₆haloalkyl, —OR⁶, and —C(═O)OR⁶. In some embodiments, the invention provides a compound of Formula (Ib), or a pharmaceutically acceptable salt or solvate thereof, wherein each R³ is independently selected from halogen and —C₁-C₆alkyl. In some embodiments, the invention provides a compound of Formula (Ib), or a pharmaceutically acceptable salt or solvate thereof, wherein each R³ is halogen. In some embodiments, the invention provides a compound of Formula (Ib), or a pharmaceutically acceptable salt or solvate thereof, wherein each R³ is —C₁-C₆alkyl. In a further embodiment, R³ is methyl. In some embodiments, the invention provides a compound of Formula (Ib), or a pharmaceutically acceptable salt or solvate thereof, wherein each R³ is —C₁-C₆haloalkyl. In a further embodiment, R³ is trifluoromethyl. In some embodiments, the invention provides a compound of Formula (Ib), or a pharmaceutically acceptable salt or solvate thereof, wherein each R³ is independently selected from —OR⁶, —N(R⁶)₂, —C(═O)OR⁶, —C(═O)N(R⁶)₂, and —SO₂N(R⁶)₂. In some embodiments, the invention provides a compound of Formula (Ib), or a pharmaceutically acceptable salt or solvate thereof, wherein each R³ is —OR⁶. In a further embodiment, R⁶ is hydrogen or methyl. In some embodiments, the invention provides a compound of Formula (Ib), or a pharmaceutically acceptable salt or solvate thereof, wherein each R³ is —OR⁶ and each R⁶ is —C₁-C₆alkyl. In some embodiments, the invention provides a compound of Formula (Ib), or a pharmaceutically acceptable salt or solvate thereof, wherein each R³ is —N(R⁶)₂. In some embodiments, the invention provides a compound of Formula (Ib), or a pharmaceutically acceptable salt or solvate thereof, wherein each R³ is —C(═O)OR⁶. In some embodiments, the invention provides a compound of Formula (Ib), or a pharmaceutically acceptable salt or solvate thereof, wherein each R³ is —C(═O)N(R⁶)₂. In some embodiments, the invention provides a compound of Formula (Ib), or a pharmaceutically acceptable salt or solvate thereof, wherein each R³ is —SO₂N(R⁶)₂.

In some embodiments, the invention provides a compound of Formula (Ib), or a pharmaceutically acceptable salt or solvate thereof, wherein q is 0. In some embodiments, the invention provides a compound of Formula (Ib), or a pharmaceutically acceptable salt or solvate thereof, wherein q is 1. In some embodiments, the invention provides a compound of Formula (Ib), or a pharmaceutically acceptable salt or solvate thereof, wherein q is 2.

In some embodiments, the invention provides a compound of Formula (Ib), or a pharmaceutically acceptable salt or solvate thereof, wherein each R⁴ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂. In some embodiments, the invention provides a compound of Formula (Ib), or a pharmaceutically acceptable salt or solvate thereof, wherein each R⁴ is independently selected from halogen, —C₁-C₆alkyl, —C₁-C₆haloalkyl, —OR⁶, and —C(═O)OR⁶. In some embodiments, the invention provides a compound of Formula (Ib), or a pharmaceutically acceptable salt or solvate thereof, wherein each R⁴ is independently selected from halogen and —C₁-C₆alkyl. In some embodiments, the invention provides a compound of Formula (Ib), or a pharmaceutically acceptable salt or solvate thereof, wherein each R⁴ is halogen. In some embodiments, the invention provides a compound of Formula (Ib), or a pharmaceutically acceptable salt or solvate thereof, wherein each R⁴ is —C₁-C₆alkyl. In a further embodiment, R⁴ is methyl. In some embodiments, the invention provides a compound of Formula (Ib), or a pharmaceutically acceptable salt or solvate thereof, wherein each R⁴ is —C₁-C₆haloalkyl. In a further embodiment, R⁴ is trifluoromethyl. In some embodiments, the invention provides a compound of Formula (Ib), or a pharmaceutically acceptable salt or solvate thereof, wherein each R⁴ is independently selected from —OR⁶, —N(R⁶)₂, —C(═O)OR⁶, —C(═O)N(R⁶)₂, and —SO₂N(R⁶)₂. In some embodiments, the invention provides a compound of Formula (Ib), or a pharmaceutically acceptable salt or solvate thereof, wherein each R⁴ is —OR⁶. In a further embodiment, R⁶ is hydrogen or methyl. In some embodiments, the invention provides a compound of Formula (Ib), or a pharmaceutically acceptable salt or solvate thereof, wherein each R⁴ is —OR⁶ and each R⁶ is —C₁-C₆alkyl. In some embodiments, the invention provides a compound of Formula (Ib), or a pharmaceutically acceptable salt or solvate thereof, wherein each R⁴ is —N(R⁶)₂. In some embodiments, the invention provides a compound of Formula (Ib), or a pharmaceutically acceptable salt or solvate thereof, wherein each R⁴ is —C(═O)OR⁶. In some embodiments, the invention provides a compound of Formula (I), or a pharmaceutically acceptable salt or solvate thereof, wherein each R⁴ is —C(═O)N(R⁶)₂. In some embodiments, the invention provides a compound of Formula (I), or a pharmaceutically acceptable salt or solvate thereof, wherein each R⁴ is —SO₂N(R⁶)₂.

In some embodiments, the invention provides a compound of Formula (Ib), or a pharmaceutically acceptable salt or solvate thereof, wherein R¹ is —C₁-C₆alkyl, —C₁-C₆haloalkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (Ib), or a pharmaceutically acceptable salt or solvate thereof, wherein R¹ is —C₁-C₆alkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (Ib), or a pharmaceutically acceptable salt or solvate thereof, wherein R¹ is —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (Ib), or a pharmaceutically acceptable salt or solvate thereof, wherein R¹ is —C(═O)OR⁶. In some embodiments, the invention provides a compound of Formula (Ib), or a pharmaceutically acceptable salt or solvate thereof, wherein R¹ is —C(═O)OR⁶ and R⁶ is —C₁-C₆alkyl, —C₁-C₆alkylene-OR⁹, or —C₁-C₆alkylene-N(R⁹)₂. In some embodiments, the invention provides a compound of Formula (Ib), or a pharmaceutically acceptable salt or solvate thereof, wherein R¹ is —C(═O)OR⁶. In some embodiments, the invention provides a compound of Formula (Ib), or a pharmaceutically acceptable salt or solvate thereof, wherein R¹ is —C(═O)OR⁶ and R⁶ is —C₁-C₆alkyl. In some embodiments, the invention provides a compound of Formula (Ib), or a pharmaceutically acceptable salt or solvate thereof, wherein R⁶ is methyl, ethyl, n-propyl, isobutyl, or t-butyl. In another embodiment, R⁶ is methyl. In yet a further embodiment, R⁶ is ethyl. In some embodiments, the invention provides a compound of Formula (Ib), or a pharmaceutically acceptable salt or solvate thereof, wherein R¹ is —C(═O)OR⁶ and R⁶ is —C₁-C₆alkylene-OR⁹. In some embodiments, the invention provides a compound of Formula (Ib), or a pharmaceutically acceptable salt or solvate thereof, wherein R¹ is —C(═O)OR⁶, R⁶ is —C₁-C₆alkylene-OR⁹, and R⁹ is —C₁-C₆alkyl. In a further embodiment, R⁶ is ethylene-OR⁹. In another embodiment, R⁶ is methylene-OR⁹. In another embodiment, R⁹ is selected from methyl, ethyl, n-propyl, or t-butyl. In one embodiment, R⁹ is methyl. In some embodiments, the invention provides a compound of Formula (Ib), or a pharmaceutically acceptable salt or solvate thereof, wherein R¹ is —C(═O)OR⁶ and R⁶ is —C₁-C₆alkylene-N(R⁹)₂. In some embodiments, the invention provides a compound of Formula (Ib), or a pharmaceutically acceptable salt or solvate thereof, wherein R¹ is —C(═O)N(R⁶)₂. In some embodiments, the invention provides a compound of Formula (Ib), or a pharmaceutically acceptable salt or solvate thereof, wherein R¹ is —C(═O)N(R⁶)₂ and each R⁶ is independently selected from hydrogen and —C₁-C₆alkyl. In some embodiments, the invention provides a compound of Formula (Ib), or a pharmaceutically acceptable salt or solvate thereof, wherein R¹ is —C(═O)N(R⁶)₂ and each R⁶ is independently selected from hydrogen and —C₁-C₆alkylene-OR⁹. In some embodiments, the invention provides a compound of Formula (Ib), or a pharmaceutically acceptable salt or solvate thereof, wherein R¹ is —S(═O)₂R⁷. In some embodiments, the invention provides a compound of Formula (Ib), or a pharmaceutically acceptable salt or solvate thereof, wherein R¹ is —S(═O)₂N(R⁶)₂. In some embodiments, the invention provides a compound of Formula (Ib), or a pharmaceutically acceptable salt or solvate thereof, wherein R¹ is —C(═O)R⁷.

In some embodiments, the invention provides a compound of Formula (Ib), or a pharmaceutically acceptable salt or solvate thereof, wherein R² is —C(═O)NH(OH), —CH₂N(OH)C(═O)R⁸, —CH(OH)N(═O), —C(═O)CF₃, —CH₂NHS(═O)₂R⁸, —CH(NH₂)C(═O)OH,

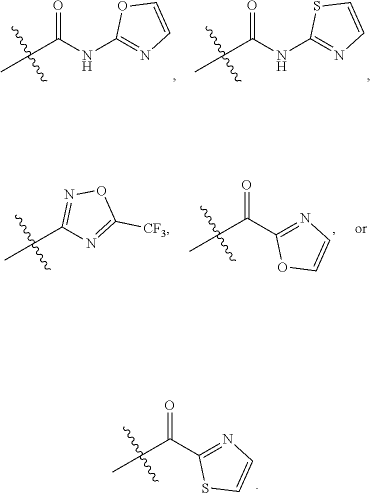 [SMILES: *C(=O)Nc1ncco1 ; *C(=O)Nc1nccs1 ; *c1noc(C(F)(F)F)n1 ; *C(=O)c1ncco1 ; *C(=O)c1nccs1]

In some embodiments, the invention provides a compound of Formula (Ib), or a pharmaceutically acceptable salt or solvate thereof, wherein R² is —C(═O)NH(OH). In some embodiments, the invention provides a compound of Formula (Ib), or a pharmaceutically acceptable salt or solvate thereof, wherein R² is —CH₂N(OH)C(═O)R⁸. In some embodiments, the invention provides a compound of Formula (Ib), or a pharmaceutically acceptable salt or solvate thereof, wherein R² is —CH(OH)N(═O). In some embodiments, the invention provides a compound of Formula (Ib), or a pharmaceutically acceptable salt or solvate thereof, wherein R² is —C(═O)CF₃. In some embodiments, the invention provides a compound of Formula (Ib), or a pharmaceutically acceptable salt or solvate thereof, wherein R² is —CH₂NHS(═O)₂R⁸. In some embodiments, the invention provides a compound of Formula (Ib), or a pharmaceutically acceptable salt or solvate thereof, wherein R² is —CH(NH₂)C(═O)OH. In some embodiments, the invention provides a compound of Formula (Ib), or a pharmaceutically acceptable salt or solvate thereof, wherein R² is

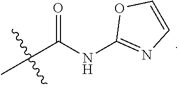 [SMILES: *C(=O)Nc1ncco1]

In some embodiments, the invention provides a compound of Formula (Ib), or a pharmaceutically acceptable salt or solvate thereof, wherein R² is

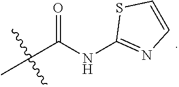 [SMILES: *C(=O)Nc1nccs1]

In some embodiments, the invention provides a compound of Formula (Ib), or a pharmaceutically acceptable salt or solvate thereof, wherein R² is

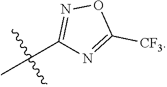 [SMILES: *c1noc(C(F)(F)F)n1]

In some embodiments, the invention provides a compound of Formula (Ib), or a pharmaceutically acceptable salt or solvate thereof, wherein R² is

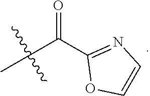 [SMILES: *C(=O)c1ncco1]

In some embodiments, the invention provides a compound of Formula (Ib), or a pharmaceutically acceptable salt or solvate thereof, wherein R² is

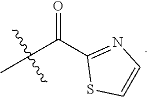 [SMILES: *C(=O)c1nccs1]

In some embodiments, the invention provides a compound of Formula (Ib), or a pharmaceutically acceptable salt or solvate thereof, wherein n is 1. In some embodiments, the invention provides a compound of Formula (Ib), or a pharmaceutically acceptable salt or solvate thereof, wherein n is 2. In some embodiments, the invention provides a compound of Formula (Ib), or a pharmaceutically acceptable salt or solvate thereof, wherein n is 0.

In some embodiments, the invention provides a compound of Formula (I), having the structure of Formula (Ic):

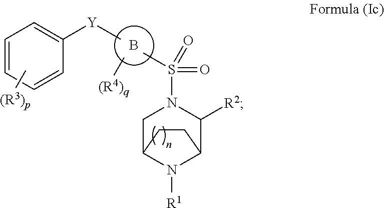

wherein:
\textcircled{A} is C₂-C₉heterocycloalkyl,
Y is a direct bond, —O—, —CH₂—, or —C(═O)—;
wherein if nitrogen is the attachment point on \textcircled{B} for attaching Y then Y cannot be —O—;
R¹ is hydrogen, —C₁-C₆alkyl, —C₁-C₆haloalkyl, —C₁-C₆alkylene-OR⁶, —C₁-C₆alkylene-N(R⁶)₂, —C₁-C₆alkylene-C(═O)—N(R⁶)₂, —C₁-C₆alkylene-C(═O)—C₄-C₅heterocycloalkyl, —C(═O)R⁷, —C(═O)OR⁶, —C(═O)N(R⁶)₂, —S(═O)₂R⁷, or —S(═O)₂N(R⁶)₂;
R² is —C(═O)NH(OH), —CH₂N(OH)C(═O)R⁸, —CH(OH)N(═O), —C(═O)CF³, —CH₂NHS(═O)₂R⁸, —CH(NH₂)C(═O)OH,

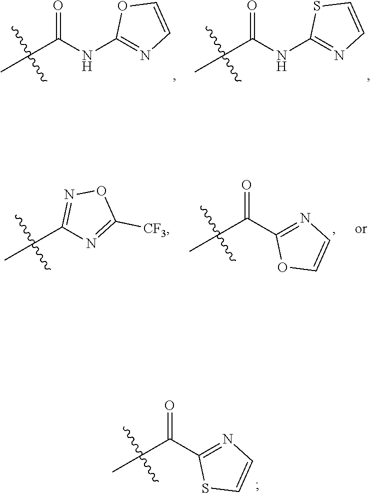 [SMILES: *C(=O)Nc1ncco1 ; *C(=O)Nc1nccs1 ; *c1noc(C(F)(F)F)n1 ; *C(=O)c1ncco1 ; *C(=O)c1nccs1]

each R³ and each R⁴ are independently selected from halogen, —C₁-C₆alkyl, —C₁-C₆haloalkyl, —C₁-C₆alkylene-OR⁶, C₁-C₆haloalkoxy, —OR, —N(R⁶)₂, —CN, —C(═O)R⁷, —C(═O)OR⁶, —C(═O)N(R⁶)₂, —NR⁶C(═O)R⁷, —NR⁶SO₂R⁷, —SO₂R⁷, —SO₂N(R⁶)₂, or phenyl optionally substituted with halogen;
each R⁶ is independently selected from hydrogen, —C₁-C₆alkyl, —C₁-C₆haloalkyl, —C₁-C₆alkylene-OR⁹, —C₁-C₆alkylene-N(R⁹)₂, and —C₁-C₆alkylene-C₂-C₉heterocycloalkyl;
or —N(R⁶)₂ represents C₃-C₅heterocycloalkyl;
each R⁷ is independently selected from —C₁-C₆alkyl, —C₁-C₆alkylene-OR⁹, —C₁-C₆alkylene-N(R⁹)₂, —C₂-C₉heterocycloalkyl, —C₂-C₉heteroaryl, —C₁-C₆alkylene-C₂-C₉heterocycloalkyl, and —C₁-C₆alkylene-C₂-C₉heteroaryl;
R⁸ is selected from hydrogen, —C₁-C₆alkyl, and —N(H)C₁-C₆alkyl;
each R⁹ is independently selected from hydrogen and —C₁-C₆alkyl;
each occurrence of C₂-C₉heterocycloalkyl and C₂-C₉heteroaryl being optionally substituted with —C₁-C₆alkyl or —C₁-C₆haloalkyl or halogen;
n is 0, 1, or 2;
p is 0, 1, 2, or 3; and
q is 0, 1, 2, or 3;
   or a pharmaceutically acceptable salt or solvate thereof.

In some embodiments, the invention provides a compound of Formula (Ic), or a pharmaceutically acceptable salt or solvate thereof, wherein Y is a direct bond. In some embodiments, the invention provides a compound of Formula (Ic), or a pharmaceutically acceptable salt or solvate thereof, wherein Y is —O—. In some embodiments, the invention provides a compound of Formula (Ic), or a pharmaceutically acceptable salt or solvate thereof, wherein Y is —CH₂—.

In some embodiments, the invention provides a compound of Formula (Ic), or a pharmaceutically acceptable salt or solvate thereof, wherein p is 0. In some embodiments, the invention provides a compound of Formula (Ic), or a pharmaceutically acceptable salt or solvate thereof, wherein p is 1. In some embodiments, the invention provides a compound of Formula (Ic), or a pharmaceutically acceptable salt or solvate thereof, wherein p is 2.

In some embodiments, the invention provides a compound of Formula (Ic), or a pharmaceutically acceptable salt or solvate thereof, wherein each R³ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂. In some embodiments, the invention provides a compound of Formula (Ic), or a pharmaceutically acceptable salt or solvate thereof, wherein each R³ is independently selected from halogen, —C₁-C₆alkyl, —C₁-C₆haloalkyl, —OR⁶, and —C(═O)OR⁶. In some embodiments, the invention provides a compound of Formula (Ic), or a pharmaceutically acceptable salt or solvate thereof, wherein each R³ is independently selected from halogen and —C₁-C₆alkyl. In some embodiments, the invention provides a compound of Formula (Ic), or a pharmaceutically acceptable salt or solvate thereof, wherein each R³ is halogen. In some embodiments, the invention provides a compound of Formula (Ic), or a pharmaceutically acceptable salt or solvate thereof, wherein each R³ is —C₁-C₆alkyl. In a further embodiment, R³ is methyl. In some embodiments, the invention provides a compound of Formula (Ic), or a pharmaceutically acceptable salt or solvate thereof, wherein each R³ is —C₁-C₆haloalkyl. In a further embodiment, R³ is trifluoromethyl. In some embodiments, the invention provides a compound of Formula (Ic), or a pharmaceutically acceptable salt or solvate thereof, wherein each R³ is independently selected from —OR⁶, —N(R⁶)₂, —C(═O)OR⁶, —C(═O)N(R⁶)₂, and —SO₂N(R⁶)₂. In some embodiments, the invention provides a compound of Formula (Ic), or a pharmaceutically acceptable salt or solvate thereof, wherein each R³ is —OR⁶. In a further embodiment, R⁶ is hydrogen or methyl. In some embodiments, the invention provides a compound of Formula (Ic), or a pharmaceutically acceptable salt or solvate thereof, wherein each R³ is —OR⁶ and each R⁶ is —C₁-C₆alkyl. In some embodiments, the invention provides a compound of Formula (Ic), or a pharmaceutically acceptable salt or solvate thereof, wherein each R³ is —N(R⁶)₂. In some embodiments, the invention provides a compound of Formula (Ic), or a pharmaceutically acceptable salt or solvate thereof, wherein each R³ is —C(═O)OR⁶. In some embodiments, the invention provides a compound of Formula (Ic), or a pharmaceutically acceptable salt or solvate thereof, wherein each R³ is —C(═O)N(R⁶)₂. In some embodiments, the invention provides a compound of Formula (Ic), or a pharmaceutically acceptable salt or solvate thereof, wherein each R³ is —SO₂N(R⁶)₂.

In some embodiments, the invention provides a compound of Formula (Ic), or a pharmaceutically acceptable salt or solvate thereof, wherein q is 0. In some embodiments, the invention provides a compound of Formula (Ic), or a pharmaceutically acceptable salt or solvate thereof, wherein q is 1. In some embodiments, the invention provides a compound of Formula (Ic), or a pharmaceutically acceptable salt or solvate thereof, wherein q is 2.

In some embodiments, the invention provides a compound of Formula (Ic), or a pharmaceutically acceptable salt or solvate thereof, wherein each R⁴ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂. In some embodiments, the invention provides a compound of Formula (Ic), or a pharmaceutically acceptable salt or solvate thereof, wherein each R⁴ is independently selected from halogen, —C₁-C₆alkyl, —C₁-C₆haloalkyl, —OR⁶, and —C(═O)OR⁶. In some embodiments, the invention provides a compound of Formula (Ic), or a pharmaceutically acceptable salt or solvate thereof, wherein each R⁴ is independently selected from halogen and —C₁-C₆alkyl. In some embodiments, the invention provides a compound of Formula (Ic), or a pharmaceutically acceptable salt or solvate thereof, wherein each R⁴ is halogen. In some embodiments, the invention provides a compound of Formula (Ic), or a pharmaceutically acceptable salt or solvate thereof, wherein each R⁴ is —C₁-C₆alkyl. In a further embodiment, R⁴ is methyl. In some embodiments, the invention provides a compound of Formula (Ic), or a pharmaceutically acceptable salt or solvate thereof, wherein each R⁴ is —C₁-C₆haloalkyl. In a further embodiment, R⁴ is trifluoromethyl. In some embodiments, the invention provides a compound of Formula (Ic), or a pharmaceutically acceptable salt or solvate thereof, wherein each R⁴ is independently selected from —OR⁶, —N(R⁶)₂, —C(═O)OR⁶, —C(═O)N(R⁶)₂, and —SO₂N(R⁶)₂. In some embodiments, the invention provides a compound of Formula (Ic), or a pharmaceutically acceptable salt or solvate thereof, wherein each R⁴ is —OR⁶. In a further embodiment, R⁶ is hydrogen or methyl. In some embodiments, the invention provides a compound of Formula (Ic), or a pharmaceutically acceptable salt or solvate thereof, wherein each R⁴ is —OR⁶ and each R⁶ is —C₁-C₆alkyl. In some embodiments, the invention provides a compound of Formula (Ic), or a pharmaceutically acceptable salt or solvate thereof, wherein each R⁴ is —N(R⁶)₂. In some embodiments, the invention provides a compound of Formula (Ic), or a pharmaceutically acceptable salt or solvate thereof, wherein each R⁴ is —C(═O)OR⁶. In some embodiments, the invention provides a compound of Formula (Ic), or a pharmaceutically acceptable salt or solvate thereof, wherein each R⁴ is —C(═O)N(R⁶)₂. In some embodiments, the invention provides a compound of Formula (Ic), or a pharmaceutically acceptable salt or solvate thereof, wherein each R⁴ is —SO₂N(R⁶)₂.

In some embodiments, the invention provides a compound of Formula (Ic), or a pharmaceutically acceptable salt or solvate thereof, wherein R¹ is —C₁-C₆alkyl, —C₁-C₆haloalkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (Ic), or a pharmaceutically acceptable salt or solvate thereof, wherein R¹ is —C₁-C₆alkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (Ic), or a pharmaceutically acceptable salt or solvate thereof, wherein R¹ is —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (Ic), or a pharmaceutically acceptable salt or solvate thereof, wherein R¹ is —C(═O)OR⁶. In some embodiments, the invention provides a compound of Formula (Ic), or a pharmaceutically acceptable salt or solvate thereof, wherein R¹ is —C(═O)OR⁶ and R⁶ is —C₁-C₆alkyl, —C₁-C₆alkylene-OR⁹, or —C₁-C₆alkylene-N(R⁹)₂. In some embodiments, the invention provides a compound of Formula (Ic), or a pharmaceutically acceptable salt or solvate thereof, wherein R¹ is —C(═O)OR⁶. In some embodiments, the invention provides a compound of Formula (Ic), or a pharmaceutically acceptable salt or solvate thereof, wherein R¹ is —C(═O)OR⁶ and R⁶ is —C₁-C₆alkyl. In some embodiments, the invention provides a compound of Formula (Ic), or a pharmaceutically acceptable salt or solvate thereof, wherein R⁶ is methyl, ethyl, n-propyl, isobutyl, or t-butyl. In another embodiment, R⁶ is methyl. In yet a further embodiment, R⁶ is ethyl. In some embodiments, the invention provides a compound of Formula (Ic), or a pharmaceutically acceptable salt or solvate thereof, wherein R¹ is —C(═O)OR⁶ and R⁶ is —C₁-C₆alkylene-OR⁹. In some embodiments, the invention provides a compound of Formula (Ic), or a pharmaceutically acceptable salt or solvate thereof, wherein R¹ is —C(═O)OR⁶, R⁶ is —C₁-C₆alkylene-OR⁹, and R⁹ is —C₁-C₆alkyl. In a further embodiment, R⁶ is ethylene-OR⁹. In another embodiment, R⁶ is methylene-OR⁹. In another embodiment, R⁹ is selected from methyl, ethyl, n-propyl, or t-butyl. In one embodiment, R⁹ is methyl. In some embodiments, the invention provides a compound of Formula (Ic), or a pharmaceutically acceptable salt or solvate thereof, wherein R¹ is —C(═O)OR⁶ and R⁶ is —C₁-C₆alkylene-N(R⁹)₂. In some embodiments, the invention provides a compound of Formula (Ic), or a pharmaceutically acceptable salt or solvate thereof, wherein R¹ is —C(═O)N(R⁶)₂. In some embodiments, the invention provides a compound of Formula (Ic), or a pharmaceutically acceptable salt or solvate thereof, wherein R¹ is —C(═O)N(R⁶)₂ and each R⁶ is independently selected from hydrogen and —C₁-C₆alkyl. In some embodiments, the invention provides a compound of Formula (Ic), or a pharmaceutically acceptable salt or solvate thereof, wherein R¹ is —C(═O)N(R⁶)₂ and each R⁶ is independently selected from hydrogen and —C₁-C₆alkylene-OR⁹. In some embodiments, the invention provides a compound of Formula (Ic), or a pharmaceutically acceptable salt or solvate thereof, wherein R¹ is —S(═O)₂R⁷. In some embodiments, the invention provides a compound of Formula (Ic), or a pharmaceutically acceptable salt or solvate thereof, wherein R¹ is —S(═O)₂N(R⁶)₂. In some embodiments, the invention provides a compound of Formula (Ic), or a pharmaceutically acceptable salt or solvate thereof, wherein R¹ is —C(═O)R⁷.

In some embodiments, the invention provides a compound of Formula (Ic), or a pharmaceutically acceptable salt or solvate thereof, wherein R² is —C(═O)NH(OH), —CH₂N(OH)C(═O)R⁸, —CH(OH)N(═O), —C(═O)CF₃, —CH₂NHS(═O)₂R⁸, —CH(NH₂)C(═O)OH,

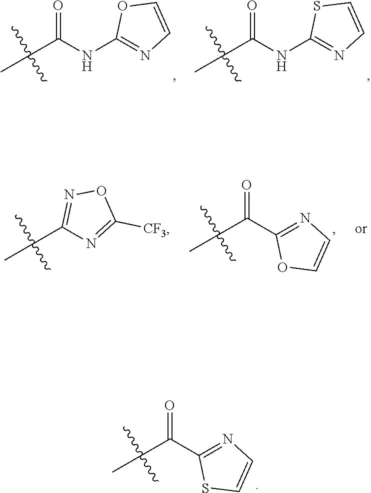 [SMILES: *C(=O)Nc1ncco1 ; *C(=O)Nc1nccs1 ; *c1noc(C(F)(F)F)n1 ; *C(=O)c1ncco1 ; *C(=O)c1nccs1]

In some embodiments, the invention provides a compound of Formula (Ic), or a pharmaceutically acceptable salt or solvate thereof, wherein R² is —C(═O)NH(OH). In some embodiments, the invention provides a compound of Formula (Ic), or a pharmaceutically acceptable salt or solvate thereof, wherein R² is —CH₂N(OH)C(═O)R⁸. In some embodiments, the invention provides a compound of Formula (Ic), or a pharmaceutically acceptable salt or solvate thereof, wherein R² is —CH(OH)N(═O). In some embodiments, the invention provides a compound of Formula (Ic), or a pharmaceutically acceptable salt or solvate thereof, wherein R² is —C(═O)CF₃. In some embodiments, the invention provides a compound of Formula (Ic), or a pharmaceutically acceptable salt or solvate thereof, wherein R² is —CH₂NHS(═O)₂R⁸. In some embodiments, the invention provides a compound of Formula (Ic), or a pharmaceutically acceptable salt or solvate thereof, wherein R² is —CH(NH₂)C(═O)OH. In some embodiments, the invention provides a compound of Formula (Ic), or a pharmaceutically acceptable salt or solvate thereof, wherein R² is

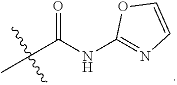 [SMILES: *C(=O)Nc1ncco1]

In some embodiments, the invention provides a compound of Formula (Ic), or a pharmaceutically acceptable salt or solvate thereof, wherein R² is

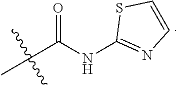 [SMILES: *C(=O)Nc1nccs1]

In some embodiments, the invention provides a compound of Formula (Ic), or a pharmaceutically acceptable salt or solvate thereof, wherein R² is

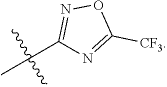 [SMILES: *c1noc(C(F)(F)F)n1]

In some embodiments, the invention provides a compound of Formula (Ic), or a pharmaceutically acceptable salt or solvate thereof, wherein R² is

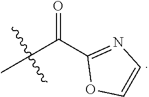 [SMILES: *C(=O)c1ncco1]

In some embodiments, the invention provides a compound of Formula (Ic), or a pharmaceutically acceptable salt or solvate thereof, wherein R² is

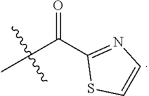 [SMILES: *C(=O)c1nccs1]

In some embodiments, the invention provides a compound of Formula (Ic), or a pharmaceutically acceptable salt or solvate thereof, wherein n is 1. In some embodiments, the invention provides a compound of Formula (Ic), or a pharmaceutically acceptable salt or solvate thereof, wherein n is 2. In some embodiments, the invention provides a compound of Formula (Ic), or a pharmaceutically acceptable salt or solvate thereof, wherein n is 0.

In some embodiments, the invention provides a compound of Formula (I), having the structure of Formula (II):

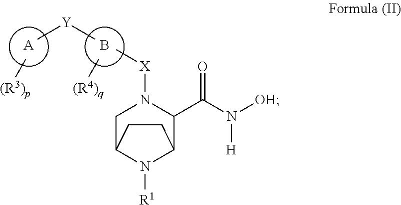

wherein:
\textcircled{A} is phenyl, C₃-C₁₀cycloalkyl, C₂-C₉heterocycloalkyl, or C₂-C₉heteroaryl;
\textcircled{B} is phenyl, C₃-C₁₀cycloalkyl, C₂-C₉heterocycloalkyl, or C₂-C₉heteroaryl;
X is —C(═O)— or —S(═O)₂—;
Y is a direct bond, —O—, —CH₂O—, —OCH₂—, —CH₂—, —C(═O)—, —C(═O)NH—, or —N(R⁵)—;
wherein Y, if asymmetrical, is written in the sequence corresponding to

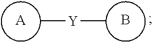

wherein if nitrogen is the attachment point on \textcircled{B} for attaching Y then Y cannot be neither —O—, —CH₂O—, —C(═O)NH—, nor —N(R⁵)—;
R¹ is hydrogen, —C₁-C₆alkyl, —C₁-C₆haloalkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂;
each R³ and each R⁴ are independently selected from halogen, —C₁-C₆alkyl, —C₁-C₆haloalkyl, —C₁-C₆alkylene-OR⁶, C₁-C₆haloalkoxy, —OR⁶, —N(R⁶)₂, —CN, —C(═O)R⁷, —C(═O)OR⁶, —C(═O)N(R⁶)₂, —NR⁶C(═O)R⁷, —NR⁶SO₂R⁷, —SO₂R⁷, —SO₂N(R⁶)₂, or phenyl optionally substituted with halogen;
R⁵ is hydrogen or —C₁-C₆alkyl;
each R⁶ is independently selected from hydrogen, —C₁-C₆alkyl, —C₁-C₆haloalkyl, —C₁-C₆alkylene-OR⁹, —C₁-C₆alkylene-N(R⁹)₂, and —C₁-C₆alkylene-C₂-C₉heterocycloalkyl;
or —N(R⁶)₂ represents C₃-C₅heterocycloalkyl;
each R⁷ is independently selected from —C₁-C₆alkyl, —C₁-C₆alkylene-OR⁹, —C₁-C₆alkylene-N(R⁹)₂, —C₂-C₉heterocycloalkyl, —C₂-C₉heteroaryl, —C₁-C₆alkylene-C₂-C₉heterocycloalkyl, and —C₁-C₆alkylene-C₂-C₉heteroaryl;
each R⁹ is independently selected from hydrogen and —C₁-C₆alkyl;
each occurrence of C₂-C₉heterocycloalkyl and C₂-C₉heteroaryl being optionally substituted with —C₁-C₆alkyl or —C₁-C₆haloalkyl or halogen;
p is 0, 1, 2, or 3; and
q is 0, 1, 2, or 3;
   or a pharmaceutically acceptable salt or solvate thereof.

In some embodiments, the invention provides a compound of Formula (II), or a pharmaceutically acceptable salt or solvate thereof, wherein \textcircled{A} is phenyl. In some embodiments, the invention provides a compound of Formula (I), or a pharmaceutically acceptable salt or solvate thereof, wherein \textcircled{A} is C₃-C₁₀cycloalkyl or C₂-C₉heteroaryl. In some embodiments, the invention provides a compound of Formula some embodiments is a compound of Formula (II), or a pharmaceutically acceptable salt or solvate thereof, wherein \textcircled{A} is C₂-C₉heteroaryl.

In some embodiments, the invention provides a compound of Formula (II), or a pharmaceutically acceptable salt or solvate thereof, wherein \textcircled{B} is phenyl. In some embodiments, the invention provides a compound of Formula (II), or a pharmaceutically acceptable salt or solvate thereof, wherein \textcircled{B} is C₃-C₁₀cycloalkyl or C₂-C₉heteroaryl. In some embodiments, the invention provides a compound of Formula (II), or a pharmaceutically acceptable salt or solvate thereof, wherein \textcircled{B} is C₂-C₉heteroaryl.

In some embodiments, the invention provides a compound of Formula (II), or a pharmaceutically acceptable salt or solvate thereof, wherein \textcircled{A} is phenyl and \textcircled{B} is phenyl.

In some embodiments, the invention provides a compound of Formula (II), or a pharmaceutically acceptable salt or solvate thereof, wherein X is —C(═O)—. In some embodiments, the invention provides a compound of Formula (II), or a pharmaceutically acceptable salt or solvate thereof, wherein X is —S(═O)₂—.

In some embodiments, the invention provides a compound of Formula (II), or a pharmaceutically acceptable salt or solvate thereof, wherein Y is —O—. In some embodiments, the invention provides a compound of Formula (II), or a pharmaceutically acceptable salt or solvate thereof, wherein Y is —CH₂O—. In some embodiments, the invention provides a compound of Formula (II), or a pharmaceutically acceptable salt or solvate thereof, wherein Y is —OCH₂—. In some embodiments, the invention provides a compound of Formula (II), or a pharmaceutically acceptable salt or solvate thereof, wherein Y is —N(R⁵)—. In some embodiments, the invention provides a compound of Formula (II), or a pharmaceutically acceptable salt or solvate thereof, wherein Y is —N(H)—. In some embodiments, the invention provides a compound of Formula (II), or a pharmaceutically acceptable salt or solvate thereof, wherein Y is —N(R⁵)— and R⁵ is —C₁-C₆alkyl. In some embodiments, the invention provides a compound of Formula (II), or a pharmaceutically acceptable salt or solvate thereof, wherein Y is —N(R⁵) and R⁵ is methyl. In some embodiments, the invention provides a compound of Formula (II), or a pharmaceutically acceptable salt or solvate thereof, wherein Y is —N(R⁵) and R⁵ is ethyl.

In some embodiments, the invention provides a compound of Formula (II), or a pharmaceutically acceptable salt or solvate thereof, wherein p is 0. In some embodiments, the invention provides a compound of Formula (II), or a pharmaceutically acceptable salt or solvate thereof, wherein p is 1. In some embodiments, the invention provides a compound of Formula (II), or a pharmaceutically acceptable salt or solvate thereof, wherein p is 2.

In some embodiments, the invention provides a compound of Formula (II), or a pharmaceutically acceptable salt or solvate thereof, wherein each R³ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂. In some embodiments, the invention provides a compound of Formula (II), or a pharmaceutically acceptable salt or solvate thereof, wherein each R³ is independently selected from halogen, —C₁-C₆alkyl, —C₁-C₆haloalkyl, —OR⁶, and —C(═O)OR⁶. In some embodiments, the invention provides a compound of Formula (II), or a pharmaceutically acceptable salt or solvate thereof, wherein each R³ is independently selected from halogen and —C₁-C₆alkyl. In some embodiments, the invention provides a compound of Formula (II), or a pharmaceutically acceptable salt or solvate thereof, wherein each R³ is halogen. In some embodiments, the invention provides a compound of Formula (II), or a pharmaceutically acceptable salt or solvate thereof, wherein each R³ is —C₁-C₆alkyl. In a further embodiment, R³ is methyl. In some embodiments, the invention provides a compound of Formula (II), or a pharmaceutically acceptable salt or solvate thereof, wherein each R³ is —C₁-C₆haloalkyl. In a further embodiment, R³ is trifluoromethyl. In some embodiments, the invention provides a compound of Formula (II), or a pharmaceutically acceptable salt or solvate thereof, wherein each R³ is independently selected from —OR⁶, —N(R⁶)₂, —C(═O)OR⁶, —C(═O)N(R⁶)₂, and —SO₂N(R⁶)₂. In some embodiments, the invention provides a compound of Formula (II), or a pharmaceutically acceptable salt or solvate thereof, wherein each R³ is —OR⁶. In a further embodiment, R⁶ is hydrogen or methyl. In some embodiments, the invention provides a compound of Formula (II), or a pharmaceutically acceptable salt or solvate thereof, wherein each R³ is —OR⁶ and each R⁶ is —C₁-C₆alkyl. In some embodiments, the invention provides a compound of Formula (II), or a pharmaceutically acceptable salt or solvate thereof, wherein each R³ is —N(R⁶)₂. In some embodiments, the invention provides a compound of Formula (II), or a pharmaceutically acceptable salt or solvate thereof, wherein each R³ is —C(═O)OR⁶. In some embodiments, the invention provides a compound of Formula (II), or a pharmaceutically acceptable salt or solvate thereof, wherein each R³ is —C(═O)N(R⁶)₂. In some embodiments, the invention provides a compound of Formula (II), or a pharmaceutically acceptable salt or solvate thereof, wherein each R³ is —SO₂N(R⁶)₂.

In some embodiments, the invention provides a compound of Formula (II), or a pharmaceutically acceptable salt or solvate thereof, wherein q is 0. In some embodiments, the invention provides a compound of Formula (II), or a pharmaceutically acceptable salt or solvate thereof, wherein q is 1. In some embodiments, the invention provides a compound of Formula (II), or a pharmaceutically acceptable salt or solvate thereof, wherein q is 2.

In some embodiments, the invention provides a compound of Formula (II), or a pharmaceutically acceptable salt or solvate thereof, wherein each R⁴ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂. In some embodiments, the invention provides a compound of Formula (II), or a pharmaceutically acceptable salt or solvate thereof, wherein each R⁴ is independently selected from halogen, —C₁-C₆alkyl, —C₁-C₆haloalkyl, —OR⁶, and —C(═O)OR⁶. In some embodiments, the invention provides a compound of Formula (II), or a pharmaceutically acceptable salt or solvate thereof, wherein each R⁴ is independently selected from halogen and —C₁-C₆alkyl. In some embodiments, the invention provides a compound of Formula (II), or a pharmaceutically acceptable salt or solvate thereof, wherein each R⁴ is halogen. In some embodiments, the invention provides a compound of Formula (II), or a pharmaceutically acceptable salt or solvate thereof, wherein each R⁴ is —C₁-C₆alkyl. In a further embodiment, R⁴ is methyl. In some embodiments, the invention provides a compound of Formula (II), or a pharmaceutically acceptable salt or solvate thereof, wherein each R⁴ is —C₁-C₆haloalkyl. In a further embodiment, R⁴ is trifluoromethyl. In some embodiments, the invention provides a compound of Formula (II), or a pharmaceutically acceptable salt or solvate thereof, wherein each R⁴ is independently selected from —OR⁶, —N(R⁶)₂, —C(═O)OR⁶, —C(═O)N(R⁶)₂, and —SO₂N(R⁶)₂. In some embodiments, the invention provides a compound of Formula (II), or a pharmaceutically acceptable salt or solvate thereof, wherein each R⁴ is —OR⁶. In a further embodiment, R⁶ is hydrogen or methyl. In some embodiments, the invention provides a compound of Formula (II), or a pharmaceutically acceptable salt or solvate thereof, wherein each R⁴ is —OR⁶ and each R⁶ is —C₁-C₆alkyl. In some embodiments, the invention provides a compound of Formula (II), or a pharmaceutically acceptable salt or solvate thereof, wherein each R⁴ is —N(R⁶)₂. In some embodiments, the invention provides a compound of Formula (II), or a pharmaceutically acceptable salt or solvate thereof, wherein each R⁴ is —C(═O)OR⁶. In some embodiments, the invention provides a compound of Formula (II), or a pharmaceutically acceptable salt or solvate thereof, wherein each R⁴ is —C(═O)N(R⁶)₂. In some embodiments, the invention provides a compound of Formula (II), or a pharmaceutically acceptable salt or solvate thereof, wherein each R⁴ is —SO₂N(R⁶)₂.

In some embodiments, the invention provides a compound of Formula (II), or a pharmaceutically acceptable salt or solvate thereof, wherein R¹ is —C₁-C₆alkyl, —C₁-C₆haloalkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (II), or a pharmaceutically acceptable salt or solvate thereof, wherein R¹ is —C₁-C₆alkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (II), or a pharmaceutically acceptable salt or solvate thereof, wherein R¹ is —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (II), or a pharmaceutically acceptable salt or solvate thereof, wherein R¹ is —C(═O)OR⁶. In some embodiments, the invention provides a compound of Formula (II), or a pharmaceutically acceptable salt or solvate thereof, wherein R¹ is —C(═O)OR⁶ and R⁶ is —C₁-C₆alkyl, —C₁-C₆alkylene-OR⁹, or —C₁-C₆alkylene-N(R⁹)₂. In some embodiments, the invention provides a compound of Formula (II), or a pharmaceutically acceptable salt or solvate thereof, wherein R¹ is —C(═O)OR⁶. In some embodiments, the invention provides a compound of Formula (II), or a pharmaceutically acceptable salt or solvate thereof, wherein R¹ is —C(═O)OR⁶ and R⁶ is —C₁-C₆alkyl. In some embodiments, the invention provides a compound of Formula (II), or a pharmaceutically acceptable salt or solvate thereof, wherein R⁶ is methyl, ethyl, n-propyl, isobutyl, or t-butyl. In another embodiment, R⁶ is methyl. In yet a further embodiment, R⁶ is ethyl. In some embodiments, the invention provides a compound of Formula (II), or a pharmaceutically acceptable salt or solvate thereof, wherein R¹ is —C(═O)OR⁶ and R⁶ is —C₁-C₆alkylene-OR⁹. In some embodiments, the invention provides a compound of Formula (II), or a pharmaceutically acceptable salt or solvate thereof, wherein R¹ is —C(═O)OR⁶, R⁶ is —C₁-C₆alkylene-OR⁹, and R⁹ is —C₁-C₆alkyl. In a further embodiment, R⁶ is ethylene-OR⁹. In another embodiment, R⁶ is methylene-OR⁹. In another embodiment, R⁹ is selected from methyl, ethyl, n-propyl, or t-butyl. In one embodiment, R⁹ is methyl. In some embodiments, the invention provides a compound of Formula (II), or a pharmaceutically acceptable salt or solvate thereof, wherein R¹ is —C(═O)OR⁶ and R⁶ is —C₁-C₆alkylene-N(R⁹)₂. In some embodiments, the invention provides a compound of Formula (II), or a pharmaceutically acceptable salt or solvate thereof, wherein R¹ is —C(═O)N(R⁶)₂. In some embodiments, the invention provides a compound of Formula (II), or a pharmaceutically acceptable salt or solvate thereof, wherein R¹ is —C(═O)N(R⁶)₂ and each R⁶ is independently selected from hydrogen and —C₁-C₆alkyl. In some embodiments, the invention provides a compound of Formula (II), or a pharmaceutically acceptable salt or solvate thereof, wherein R¹ is —C(═O)N(R⁶)₂ and each R⁶ is independently selected from hydrogen and —C₁-C₆alkylene-OR⁹. In some embodiments, the invention provides a compound of Formula (II), or a pharmaceutically acceptable salt or solvate thereof, wherein R¹ is —S(═O)₂R⁷. In some embodiments, the invention provides a compound of Formula (II), or a pharmaceutically acceptable salt or solvate thereof, wherein R¹ is —S(═O)₂N(R⁶)₂. In some embodiments, the invention provides a compound of Formula (II), or a pharmaceutically acceptable salt or solvate thereof, wherein R¹ is —C(═O)R⁷.

In some embodiments, the invention provides a compound of Formula (I), having the structure of Formula (III):

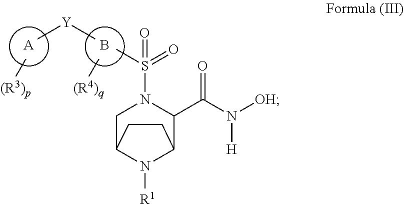

wherein:
\textcircled{A} is phenyl, C₃-C₁₀cycloalkyl, C₂-C₉heterocycloalkyl, or C₂-C₉heteroaryl;
\textcircled{B} is phenyl, C₃-C₁₀cycloalkyl, C₂-C₉heterocycloalkyl, or C₂-C₉heteroaryl;
Y is a direct bond, —O—, —CH₂O—, —OCH₂—, —CH₂—, —C(═O)—, —C(═O)NH—, or —N(R⁵)—;
wherein Y, if asymmetrical, is written in the sequence corresponding to

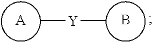

wherein if nitrogen is the attachment point on \textcircled{B} for attaching Y then Y cannot be neither —O—, —CH₂O—, —C(═O)NH—, nor —N(R⁵)—;
R¹ is hydrogen, —C₁-C₆alkyl, —C₁-C₆haloalkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂;
each R³ and each R⁴ are independently selected from halogen, —C₁-C₆alkyl, —C₁-C₆haloalkyl, —C₁-C₆alkylene-OR⁶, C₁-C₆haloalkoxy, —OR⁶, —N(R⁶)₂, —CN, —C(═O)R⁷, —C(═O)OR⁶, —C(═O)N(R⁶)₂, —NR⁶C(═O)R⁷, —NR⁶SO₂R⁷, —SO₂R⁷, —SO₂N(R⁶)₂, or phenyl optionally substituted with halogen;
R⁵ is hydrogen or —C₁-C₆alkyl;
each R⁶ is independently selected from hydrogen, —C₁-C₆alkyl, —C₁-C₆haloalkyl, —C₁-C₆alkylene-OR⁹, —C₁-C₆alkylene-N(R⁹)₂, and —C₁-C₆alkylene-C₂-C₉heterocycloalkyl;
or —N(R⁶)₂ represents C₃-C₅heterocycloalkyl;
each R⁷ is independently selected from —C₁-C₆alkyl, —C₁-C₆alkylene-OR⁹, —C₁-C₆alkylene-N(R⁹)₂, —C₂-C₉heterocycloalkyl, —C₂-C₉heteroaryl, —C₁-C₆alkylene-C₂-C₉heterocycloalkyl, and —C₁-C₆alkylene-C₂-C₉heteroaryl;
each R⁹ is independently selected from hydrogen and —C₁-C₆alkyl;
each occurrence of C₂-C₉heterocycloalkyl and C₂-C₉heteroaryl being optionally substituted with —C₁-C₆alkyl, halo-C₁-C₆alkyl or halogen;
p is 0, 1, 2, or 3; and
q is 0, 1, 2, or 3;
   or a pharmaceutically acceptable salt or solvate thereof.

In some embodiments, the invention provides a compound of Formula (III), or a pharmaceutically acceptable salt or solvate thereof, wherein \textcircled{A} is phenyl. In some embodiments, the invention provides a compound of Formula (I), or a pharmaceutically acceptable salt or solvate thereof, wherein \textcircled{A} is C₃-C₁₀cycloalkyl or C₂-C₉heteroaryl. In some embodiments, the invention provides a compound of Formula some embodiments is a compound of Formula (III), or a pharmaceutically acceptable salt or solvate thereof, wherein \textcircled{A} is C₂-C₉heteroaryl.

In some embodiments, the invention provides a compound of Formula (III), or a pharmaceutically acceptable salt or solvate thereof, wherein \textcircled{B} is phenyl. In some embodiments, the invention provides a compound of Formula (IT), or a pharmaceutically acceptable salt or solvate thereof, wherein \textcircled{B} is C₃-C₁₀cycloalkyl or C₂-C₉heteroaryl. In some embodiments, the invention provides a compound of Formula (III), or a pharmaceutically acceptable salt or solvate thereof, wherein \textcircled{B} is C₂-C₉heteroaryl.

In some embodiments, the invention provides a compound of Formula (III), or a pharmaceutically acceptable salt or solvate thereof, wherein \textcircled{A} is phenyl and \textcircled{B} is phenyl.

In some embodiments, the invention provides a compound of Formula (III), or a pharmaceutically acceptable salt or solvate thereof, wherein Y is —O—. In some embodiments, the invention provides a compound of Formula (III), or a pharmaceutically acceptable salt or solvate thereof, wherein Y is —CH₂O—. In some embodiments, the invention provides a compound of Formula (III), or a pharmaceutically acceptable salt or solvate thereof, wherein Y is —OCH₂—. In some embodiments, the invention provides a compound of Formula (III), or a pharmaceutically acceptable salt or solvate thereof, wherein Y is —N(R⁵)—. In some embodiments, the invention provides a compound of Formula (III), or a pharmaceutically acceptable salt or solvate thereof, wherein Y is —N(H)—. In some embodiments, the invention provides a compound of Formula (III), or a pharmaceutically acceptable salt or solvate thereof, wherein Y is —N(R⁵)— and R⁵ is —C₁-C₆alkyl. In some embodiments, the invention provides a compound of Formula (III), or a pharmaceutically acceptable salt or solvate thereof, wherein Y is —N(R⁵) and R⁵ is methyl. In some embodiments, the invention provides a compound of Formula (III), or a pharmaceutically acceptable salt or solvate thereof, wherein Y is —N(R⁵) and R⁵ is ethyl.

In some embodiments, the invention provides a compound of Formula (III), or a pharmaceutically acceptable salt or solvate thereof, wherein p is 0. In some embodiments, the invention provides a compound of Formula (III), or a pharmaceutically acceptable salt or solvate thereof, wherein p is 1. In some embodiments, the invention provides a compound of Formula (III), or a pharmaceutically acceptable salt or solvate thereof, wherein p is 2.

In some embodiments, the invention provides a compound of Formula (III), or a pharmaceutically acceptable salt or solvate thereof, wherein each R³ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂. In some embodiments, the invention provides a compound of Formula (III), or a pharmaceutically acceptable salt or solvate thereof, wherein each R³ is independently selected from halogen, —C₁-C₆alkyl, —C₁-C₆haloalkyl, —OR⁶, and —C(═O)OR⁶. In some embodiments, the invention provides a compound of Formula (III), or a pharmaceutically acceptable salt or solvate thereof, wherein each R³ is independently selected from halogen and —C₁-C₆alkyl. In some embodiments, the invention provides a compound of Formula (III), or a pharmaceutically acceptable salt or solvate thereof, wherein each R³ is halogen. In some embodiments, the invention provides a compound of Formula (III), or a pharmaceutically acceptable salt or solvate thereof, wherein each R³ is —C₁-C₆alkyl. In a further embodiment, R³ is methyl. In some embodiments, the invention provides a compound of Formula (III), or a pharmaceutically acceptable salt or solvate thereof, wherein each R³ is —C₁-C₆haloalkyl. In a further embodiment, R³ is trifluoromethyl. In some embodiments, the invention provides a compound of Formula (III), or a pharmaceutically acceptable salt or solvate thereof, wherein each R³ is independently selected from —OR⁶, —N(R⁶)₂, —C(═O)OR⁶, —C(═O)N(R⁶)₂, and —SO₂N(R⁶)₂. In some embodiments, the invention provides a compound of Formula (III), or a pharmaceutically acceptable salt or solvate thereof, wherein each R³ is —OR⁶. In a further embodiment, R⁶ is hydrogen or methyl. In some embodiments, the invention provides a compound of Formula (III), or a pharmaceutically acceptable salt or solvate thereof, wherein each R³ is —OR⁶ and each R⁶ is —C₁-C₆alkyl. In some embodiments, the invention provides a compound of Formula (III), or a pharmaceutically acceptable salt or solvate thereof, wherein each R³ is —N(R⁶)₂. In some embodiments, the invention provides a compound of Formula (III), or a pharmaceutically acceptable salt or solvate thereof, wherein each R³ is —C(═O)OR⁶. In some embodiments, the invention provides a compound of Formula (III), or a pharmaceutically acceptable salt or solvate thereof, wherein each R³ is —C(═O)N(R⁶)₂. In some embodiments, the invention provides a compound of Formula (III), or a pharmaceutically acceptable salt or solvate thereof, wherein each R³ is —SO₂N(R⁶)₂.

In some embodiments, the invention provides a compound of Formula (III), or a pharmaceutically acceptable salt or solvate thereof, wherein q is 0. In some embodiments, the invention provides a compound of Formula (III), or a pharmaceutically acceptable salt or solvate thereof, wherein q is 1. In some embodiments, the invention provides a compound of Formula (III), or a pharmaceutically acceptable salt or solvate thereof, wherein q is 2.

In some embodiments, the invention provides a compound of Formula (III), or a pharmaceutically acceptable salt or solvate thereof, wherein each R⁴ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂. In some embodiments, the invention provides a compound of Formula (III), or a pharmaceutically acceptable salt or solvate thereof, wherein each R⁴ is independently selected from halogen, —C₁-C₆alkyl, —C₁-C₆haloalkyl, —OR⁶, and —C(═O)OR⁶. In some embodiments, the invention provides a compound of Formula (III), or a pharmaceutically acceptable salt or solvate thereof, wherein each R⁴ is independently selected from halogen and -C₁-C₆alkyl. In some embodiments, the invention provides a compound of Formula (III), or a pharmaceutically acceptable salt or solvate thereof, wherein each R⁴ is halogen. In some embodiments, the invention provides a compound of Formula (III), or a pharmaceutically acceptable salt or solvate thereof, wherein each R⁴ is —C₁-C₆alkyl. In a further embodiment, R⁴ is methyl. In some embodiments, the invention provides a compound of Formula (III), or a pharmaceutically acceptable salt or solvate thereof, wherein each R⁴ is —C₁-C₆haloalkyl. In a further embodiment, R⁴ is trifluoromethyl. In some embodiments, the invention provides a compound of Formula (III), or a pharmaceutically acceptable salt or solvate thereof, wherein each R⁴ is independently selected from —OR⁶, —N(R⁶)₂, —C(═O)OR⁶, —C(═O)N(R⁶)₂, and —SO₂N(R⁶)₂. In some embodiments, the invention provides a compound of Formula (III), or a pharmaceutically acceptable salt or solvate thereof, wherein each R⁴ is —OR⁶. In a further embodiment, R⁶ is hydrogen or methyl. In some embodiments, the invention provides a compound of Formula (III), or a pharmaceutically acceptable salt or solvate thereof, wherein each R⁴ is —OR⁶ and each R⁶ is —C₁-C₆alkyl. In some embodiments, the invention provides a compound of Formula (III), or a pharmaceutically acceptable salt or solvate thereof, wherein each R⁴ is —N(R⁶)₂. In some embodiments, the invention provides a compound of Formula (III), or a pharmaceutically acceptable salt or solvate thereof, wherein each R⁴ is —C(═O)OR⁶. In some embodiments, the invention provides a compound of Formula (III), or a pharmaceutically acceptable salt or solvate thereof, wherein each R⁴ is —C(═O)N(R⁶)₂. In some embodiments, the invention provides a compound of Formula (III), or a pharmaceutically acceptable salt or solvate thereof, wherein each R⁴ is —SO₂N(R⁶)₂.

In some embodiments, the invention provides a compound of Formula (III), or a pharmaceutically acceptable salt or solvate thereof, wherein R¹ is —C₁-C₆alkyl, —C₁-C₆haloalkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (III), or a pharmaceutically acceptable salt or solvate thereof, wherein R¹ is —C₁-C₆alkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (III), or a pharmaceutically acceptable salt or solvate thereof, wherein R¹ is —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (III), or a pharmaceutically acceptable salt or solvate thereof, wherein R¹ is —C(═O)OR⁶. In some embodiments, the invention provides a compound of Formula (III), or a pharmaceutically acceptable salt or solvate thereof, wherein R¹ is —C(═O)OR⁶ and R⁶ is —C₁-C₆alkyl, —C₁-C₆alkylene-OR⁹, or —C₁-C₆alkylene-N(R⁹)₂. In some embodiments, the invention provides a compound of Formula (III), or a pharmaceutically acceptable salt or solvate thereof, wherein R¹ is —C(═O)OR⁶. In some embodiments, the invention provides a compound of Formula (III), or a pharmaceutically acceptable salt or solvate thereof, wherein R¹ is —C(═O)OR⁶ and R⁶ is —C₁-C₆alkyl. In some embodiments, the invention provides a compound of Formula (III), or a pharmaceutically acceptable salt or solvate thereof, wherein R⁶ is methyl, ethyl, n-propyl, isobutyl, or t-butyl. In another embodiment, R⁶ is methyl. In yet a further embodiment, R⁶ is ethyl. In some embodiments, the invention provides a compound of Formula (III), or a pharmaceutically acceptable salt or solvate thereof, wherein R¹ is —C(═O)OR⁶ and R⁶ is —C₁-C₆alkylene-OR⁹. In some embodiments, the invention provides a compound of Formula (III), or a pharmaceutically acceptable salt or solvate thereof, wherein R¹ is —C(═O)OR⁶, R⁶ is —C₁-C₆alkylene-OR⁹, and R⁹ is —C₁-C₆alkyl. In a further embodiment, R⁶ is ethylene-OR⁹. In another embodiment, R⁶ is methylene-OR⁹. In another embodiment, R⁹ is selected from methyl, ethyl, n-propyl, or t-butyl. In one embodiment, R⁹ is methyl. In some embodiments, the invention provides a compound of Formula (III), or a pharmaceutically acceptable salt or solvate thereof, wherein R¹ is —C(═O)OR⁶ and R⁶ is —C₁-C₆alkylene-N(R⁹)₂. In some embodiments, the invention provides a compound of Formula (III), or a pharmaceutically acceptable salt or solvate thereof, wherein R¹ is —C(═O)N(R⁶)₂. In some embodiments, the invention provides a compound of Formula (III), or a pharmaceutically acceptable salt or solvate thereof, wherein R¹ is —C(═O)N(R⁶)₂ and each R⁶ is independently selected from hydrogen and —C₁-C₆alkyl. In some embodiments, the invention provides a compound of Formula (III), or a pharmaceutically acceptable salt or solvate thereof, wherein R¹ is —C(═O)N(R⁶)₂ and each R⁶ is independently selected from hydrogen and —C₁-C₆alkylene-OR⁹. In some embodiments, the invention provides a compound of Formula (III), or a pharmaceutically acceptable salt or solvate thereof, wherein R¹ is —S(═O)₂R⁷. In some embodiments, the invention provides a compound of Formula (III), or a pharmaceutically acceptable salt or solvate thereof, wherein R¹ is —S(═O)₂N(R⁶)₂. In some embodiments, the invention provides a compound of Formula (III), or a pharmaceutically acceptable salt or solvate thereof, wherein R¹ is —C(═O)R⁷.

In some embodiments, the invention provides a compound of Formula (I), having the structure of Formula (IIIa):

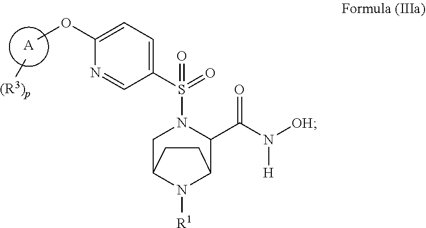

wherein:
\textcircled{A} is phenyl, C₃-C₁₀cycloalkyl, C₂-C₉heterocycloalkyl, or C₂-C₉heteroaryl;
R¹ is hydrogen, —C₁-C₆alkyl, —C₁-C₆haloalkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂;
each R³ is independently selected from halogen, —C₁-C₆alkyl, —C₁-C₆haloalkyl, —C₁-C₆alkylene-OR⁶, C₁-C₆haloalkoxy, —OR⁶, —N(R⁶)₂, —CN, —C(═O)R⁷, —C(═O)OR⁶, —C(═O)N(R⁶)₂, —NR⁶C(═O)R⁷, —NR⁶SO₂R⁷, —SO₂R⁷, —SO₂N(R⁶)₂, or phenyl optionally substituted with halogen;
each R⁶ is independently selected from hydrogen, —C₁-C₆alkyl, —C₁-C₆haloalkyl, —C₁-C₆alkylene-OR⁹, —C₁-C₆alkylene-N(R⁹)₂, and —C₁-C₆alkylene-C₂-C₉heterocycloalkyl;
or —N(R⁶)₂ represents C₃-C₅heterocycloalkyl;
each R⁷ is independently selected from —C₁-C₆alkyl, —C₁-C₆alkylene-OR⁹, —C₁-C₆alkylene-N(R⁹)₂, —C₂-C₉heterocycloalkyl, —C₂-C₉heteroaryl, —C₁-C₆alkylene-C₂-C₉heterocycloalkyl, and —C₁-C₆alkylene-C₂-C₉heteroaryl;
each R⁹ is independently selected from hydrogen and —C₁-C₆alkyl;
each occurrence of C₂-C₉heterocycloalkyl and C₂-C₉heteroaryl being optionally substituted with —C₁-C₆alkyl, halo-C₁-C₆alkyl or halogen; and
p is 0, 1, 2, or 3;
   or a pharmaceutically acceptable salt or solvate thereof.

In some embodiments, the invention provides a compound of Formula (IIIa), or a pharmaceutically acceptable salt or solvate thereof, wherein \textcircled{A} is phenyl. In some embodiments, the invention provides a compound of Formula (I), or a pharmaceutically acceptable salt or solvate thereof, wherein \textcircled{A} is C₃-C₁₀cycloalkyl or C₂-C₉heteroaryl. In some embodiments, the invention provides a compound of Formula some embodiments is a compound of Formula (IIIa), or a pharmaceutically acceptable salt or solvate thereof, wherein \textcircled{A} is C₂-C₉heteroaryl.

In some embodiments, the invention provides a compound of Formula (IIIa), or a pharmaceutically acceptable salt or solvate thereof, wherein p is 0. In some embodiments, the invention provides a compound of Formula (IIIa), or a pharmaceutically acceptable salt or solvate thereof, wherein p is 1. In some embodiments, the invention provides a compound of Formula (IIIa), or a pharmaceutically acceptable salt or solvate thereof, wherein p is 2.

In some embodiments, the invention provides a compound of Formula (IIIa), or a pharmaceutically acceptable salt or solvate thereof, wherein each R³ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂. In some embodiments, the invention provides a compound of Formula (IIIa), or a pharmaceutically acceptable salt or solvate thereof, wherein each R³ is independently selected from halogen, —C₁-C₆alkyl, —C₁-C₆haloalkyl, —OR⁶, and —C(═O)OR⁶. In some embodiments, the invention provides a compound of Formula (IIIa), or a pharmaceutically acceptable salt or solvate thereof, wherein each R³ is independently selected from halogen and —C₁-C₆alkyl. In some embodiments, the invention provides a compound of Formula (IIIa), or a pharmaceutically acceptable salt or solvate thereof, wherein each R³ is halogen. In some embodiments, the invention provides a compound of Formula (IIIa), or a pharmaceutically acceptable salt or solvate thereof, wherein each R³ is —C₁-C₆alkyl. In a further embodiment, R³ is methyl. In some embodiments, the invention provides a compound of Formula (IIIa), or a pharmaceutically acceptable salt or solvate thereof, wherein each R³ is —C₁-C₆haloalkyl. In a further embodiment, R³ is trifluoromethyl. In some embodiments, the invention provides a compound of Formula (IIIa), or a pharmaceutically acceptable salt or solvate thereof, wherein each R³ is independently selected from —OR⁶, —N(R⁶)₂, —C(═O)OR⁶, —C(═O)N(R⁶)₂, and —SO₂N(R⁶)₂. In some embodiments, the invention provides a compound of Formula (IIIa), or a pharmaceutically acceptable salt or solvate thereof, wherein each R³ is —OR⁶. In a further embodiment, R⁶ is hydrogen or methyl. In some embodiments, the invention provides a compound of Formula (IIIa), or a pharmaceutically acceptable salt or solvate thereof, wherein each R³ is —OR⁶ and each R⁶ is —C₁-C₆alkyl. In some embodiments, the invention provides a compound of Formula (IIIa), or a pharmaceutically acceptable salt or solvate thereof, wherein each R³ is —N(R⁶)₂. In some embodiments, the invention provides a compound of Formula (IIIa), or a pharmaceutically acceptable salt or solvate thereof, wherein each R³ is —C(═O)OR⁶. In some embodiments, the invention provides a compound of Formula (IIIa), or a pharmaceutically acceptable salt or solvate thereof, wherein each R³ is —C(═O)N(R⁶)₂. In some embodiments, the invention provides a compound of Formula (IIIa), or a pharmaceutically acceptable salt or solvate thereof, wherein each R³ is —SO₂N(R⁶)₂.

In some embodiments, the invention provides a compound of Formula (IIIa), or a pharmaceutically acceptable salt or solvate thereof, wherein R¹ is —C₁-C₆alkyl, —C₁-C₆haloalkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (IIIa), or a pharmaceutically acceptable salt or solvate thereof, wherein R¹ is —C₁-C₆alkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (IIIa), or a pharmaceutically acceptable salt or solvate thereof, wherein R¹ is —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (IIIa), or a pharmaceutically acceptable salt or solvate thereof, wherein R¹ is —C(═O)OR⁶. In some embodiments, the invention provides a compound of Formula (IIIa), or a pharmaceutically acceptable salt or solvate thereof, wherein R¹ is —C(═O)OR⁶ and R⁶ is —C₁-C₆alkyl, —C₁-C₆alkylene-OR⁹, or —C₁-C₆alkylene-N(R⁹)₂. In some embodiments, the invention provides a compound of Formula (IIIa), or a pharmaceutically acceptable salt or solvate thereof, wherein R¹ is —C(═O)OR⁶. In some embodiments, the invention provides a compound of Formula (IIIa), or a pharmaceutically acceptable salt or solvate thereof, wherein R¹ is —C(═O)OR⁶ and R⁶ is —C₁-C₆alkyl. In some embodiments, the invention provides a compound of Formula (IIIa), or a pharmaceutically acceptable salt or solvate thereof, wherein R⁶ is methyl, ethyl, n-propyl, isobutyl, or t-butyl. In another embodiment, R⁶ is methyl. In yet a further embodiment, R⁶ is ethyl. In some embodiments, the invention provides a compound of Formula (IIIa), or a pharmaceutically acceptable salt or solvate thereof, wherein R¹ is —C(═O)OR⁶ and R⁶ is —C₁-C₆alkylene-OR⁹. In some embodiments, the invention provides a compound of Formula (IIIa), or a pharmaceutically acceptable salt or solvate thereof, wherein R¹ is —C(═O)OR⁶, R⁶ is —C₁-C₆alkylene-OR⁹, and R⁹ is —C₁-C₆alkyl. In a further embodiment, R⁶ is ethylene-OR⁹. In another embodiment, R⁶ is methylene-OR⁹. In another embodiment, R⁹ is selected from methyl, ethyl, n-propyl, or t-butyl. In one embodiment, R⁹ is methyl. In some embodiments, the invention provides a compound of Formula (IIIa), or a pharmaceutically acceptable salt or solvate thereof, wherein R¹ is —C(═O)OR⁶ and R⁶ is —C₁-C₆alkylene-N(R⁹)₂. In some embodiments, the invention provides a compound of Formula (IIIa), or a pharmaceutically acceptable salt or solvate thereof, wherein R¹ is —C(═O)N(R⁶)₂. In some embodiments, the invention provides a compound of Formula (IIIa), or a pharmaceutically acceptable salt or solvate thereof, wherein R¹ is —C(═O)N(R⁶)₂ and each R⁶ is independently selected from hydrogen and —C₁-C₆alkyl. In some embodiments, the invention provides a compound of Formula (IIIa), or a pharmaceutically acceptable salt or solvate thereof, wherein R¹ is —C(═O)N(R⁶)₂ and each R⁶ is independently selected from hydrogen and —C₁-C₆alkylene-OR⁹. In some embodiments, the invention provides a compound of Formula (IIIa), or a pharmaceutically acceptable salt or solvate thereof, wherein R¹ is —S(═O)₂R⁷. In some embodiments, the invention provides a compound of Formula (IIIa), or a pharmaceutically acceptable salt or solvate thereof, wherein R¹ is —S(═O)₂N(R⁶)₂. In some embodiments, the invention provides a compound of Formula (IIIa), or a pharmaceutically acceptable salt or solvate thereof, wherein R¹ is —C(═O)R⁷.

In some embodiments, the invention provides a compound of Formula (I), having the structure of Formula (IIIb):

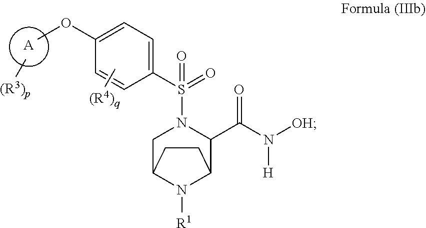

wherein:
\textcircled{A} is phenyl, C₃-C₁₀cycloalkyl, C₂-C₉heterocycloalkyl, or C₂-C₉heteroaryl;
R¹ is hydrogen, —C₁-C₆alkyl, —C₁-C₆haloalkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂;
each R³ and each R⁴ are independently selected from halogen, —C₁-C₆alkyl, —C₁-C₆haloalkyl, —C₁-C₆alkylene-OR⁶, C₁-C₆haloalkoxy, —OR⁶, —N(R⁶)₂, —CN, —C(═O)R⁷, —C(═O)OR⁶, —C(═O)N(R⁶)₂, —NR⁶C(═O)R⁷, —NR⁶SO₂R⁷, —SO₂R⁷, —SO₂N(R⁶)₂, or phenyl optionally substituted with halogen;
each R⁶ is independently selected from hydrogen, —C₁-C₆alkyl, —C₁-C₆haloalkyl, —C₁-C₆alkylene-OR⁹, —C₁-C₆alkylene-N(R⁹)₂, and —C₁-C₆alkylene-C₂-C₉heterocycloalkyl;
or —N(R⁶)₂ represents C₃-C₅heterocycloalkyl;
each R⁷ is independently selected from —C₁-C₆alkyl, —C₁-C₆alkylene-OR⁹, —C₁-C₆alkylene-N(R⁹)₂, —C₂-C₉heterocycloalkyl, —C₂-C₉heteroaryl, —C₁-C₆alkylene-C₂-C₉heterocycloalkyl, and —C₁-C₆alkylene-C₂-C₉heteroaryl;
each R⁹ is independently selected from hydrogen and —C₁-C₆alkyl;
each occurrence of C₂-C₉heterocycloalkyl and C₂-C₉heteroaryl being optionally substituted with —C₁-C₆alkyl, halo-C₁-C₆alkyl or halogen;
p is 0, 1, 2, or 3; and
q is 0, 1, 2, or 3;
   or a pharmaceutically acceptable salt or solvate thereof.

In some embodiments, the invention provides a compound of Formula (IIIb), or a pharmaceutically acceptable salt or solvate thereof, wherein \textcircled{A} is phenyl. In some embodiments, the invention provides a compound of Formula (I), or a pharmaceutically acceptable salt or solvate thereof, wherein \textcircled{A} is C₃-C₁₀cycloalkyl or C₂-C₉heteroaryl. In some embodiments, the invention provides a compound of Formula some embodiments is a compound of Formula (IIIb), or a pharmaceutically acceptable salt or solvate thereof, wherein \textcircled{A} is C₂-C₉heteroaryl.

In some embodiments, the invention provides a compound of Formula (IIIb), or a pharmaceutically acceptable salt or solvate thereof, wherein p is 0. In some embodiments, the invention provides a compound of Formula (IIIb), or a pharmaceutically acceptable salt or solvate thereof, wherein p is 1. In some embodiments, the invention provides a compound of Formula (IIIb), or a pharmaceutically acceptable salt or solvate thereof, wherein p is 2.

In some embodiments, the invention provides a compound of Formula (IIIb), or a pharmaceutically acceptable salt or solvate thereof, wherein each R³ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂. In some embodiments, the invention provides a compound of Formula (IIIb), or a pharmaceutically acceptable salt or solvate thereof, wherein each R³ is independently selected from halogen, —C₁-C₆alkyl, —C₁-C₆haloalkyl, —OR⁶, and —C(═O)OR⁶. In some embodiments, the invention provides a compound of Formula (IIIb), or a pharmaceutically acceptable salt or solvate thereof, wherein each R³ is independently selected from halogen and —C₁-C₆alkyl. In some embodiments, the invention provides a compound of Formula (IIIb), or a pharmaceutically acceptable salt or solvate thereof, wherein each R³ is halogen. In some embodiments, the invention provides a compound of Formula (IIIb), or a pharmaceutically acceptable salt or solvate thereof, wherein each R³ is —C₁-C₆alkyl. In a further embodiment, R³ is methyl. In some embodiments, the invention provides a compound of Formula (IIIb), or a pharmaceutically acceptable salt or solvate thereof, wherein each R³ is —C₁-C₆haloalkyl. In a further embodiment, R³ is trifluoromethyl. In some embodiments, the invention provides a compound of Formula (IIIb), or a pharmaceutically acceptable salt or solvate thereof, wherein each R³ is independently selected from —OR⁶, —N(R⁶)₂, —C(═O)OR⁶, —C(═O)N(R⁶)₂, and —SO₂N(R⁶)₂. In some embodiments, the invention provides a compound of Formula (IIIb), or a pharmaceutically acceptable salt or solvate thereof, wherein each R³ is —OR⁶. In a further embodiment, R⁶ is hydrogen or methyl. In some embodiments, the invention provides a compound of Formula (IIIb), or a pharmaceutically acceptable salt or solvate thereof, wherein each R³ is —OR⁶ and each R⁶ is —C₁-C₆alkyl. In some embodiments, the invention provides a compound of Formula (IIIb), or a pharmaceutically acceptable salt or solvate thereof, wherein each R³ is —N(R⁶)₂. In some embodiments, the invention provides a compound of Formula (IIIb), or a pharmaceutically acceptable salt or solvate thereof, wherein each R³ is —C(═O)OR⁶. In some embodiments, the invention provides a compound of Formula (IIIb), or a pharmaceutically acceptable salt or solvate thereof, wherein each R³ is —C(═O)N(R⁶)₂. In some embodiments, the invention provides a compound of Formula (IIIb), or a pharmaceutically acceptable salt or solvate thereof, wherein each R³ is —SO₂N(R⁶)₂.

In some embodiments, the invention provides a compound of Formula (IIIb), or a pharmaceutically acceptable salt or solvate thereof, wherein q is 0. In some embodiments, the invention provides a compound of Formula (IIIb), or a pharmaceutically acceptable salt or solvate thereof, wherein q is 1. In some embodiments, the invention provides a compound of Formula (IIIb), or a pharmaceutically acceptable salt or solvate thereof, wherein q is 2.

In some embodiments, the invention provides a compound of Formula (IIIb), or a pharmaceutically acceptable salt or solvate thereof, wherein each R⁴ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂. In some embodiments, the invention provides a compound of Formula (IIIb), or a pharmaceutically acceptable salt or solvate thereof, wherein each R⁴ is independently selected from halogen, —C₁-C₆alkyl, —C₁-C₆haloalkyl, —OR⁶, and —C(═O)OR⁶. In some embodiments, the invention provides a compound of Formula (IIIb), or a pharmaceutically acceptable salt or solvate thereof, wherein each R⁴ is independently selected from halogen and —C₁-C₆alkyl. In some embodiments, the invention provides a compound of Formula (IIIb), or a pharmaceutically acceptable salt or solvate thereof, wherein each R⁴ is halogen. In some embodiments, the invention provides a compound of Formula (IIIb), or a pharmaceutically acceptable salt or solvate thereof, wherein each R⁴ is —C₁-C₆alkyl. In a further embodiment, R⁴ is methyl. In some embodiments, the invention provides a compound of Formula (IIIb), or a pharmaceutically acceptable salt or solvate thereof, wherein each R⁴ is —C₁-C₆haloalkyl. In a further embodiment, R⁴ is trifluoromethyl. In some embodiments, the invention provides a compound of Formula (IIIb), or a pharmaceutically acceptable salt or solvate thereof, wherein each R⁴ is independently selected from —OR⁶, —N(R⁶)₂, —C(═O)OR⁶, —C(═O)N(R⁶)₂, and —SO₂N(R⁶)₂. In some embodiments, the invention provides a compound of Formula (IIIb), or a pharmaceutically acceptable salt or solvate thereof, wherein each R⁴ is —OR⁶. In a further embodiment, R⁶ is hydrogen or methyl. In some embodiments, the invention provides a compound of Formula (IIIb), or a pharmaceutically acceptable salt or solvate thereof, wherein each R⁴ is —OR⁶ and each R⁶ is —C₁-C₆alkyl. In some embodiments, the invention provides a compound of Formula (IIIb), or a pharmaceutically acceptable salt or solvate thereof, wherein each R⁴ is —N(R⁶)₂. In some embodiments, the invention provides a compound of Formula (IIIb), or a pharmaceutically acceptable salt or solvate thereof, wherein each R⁴ is —C(═O)OR⁶. In some embodiments, the invention provides a compound of Formula (IIIb), or a pharmaceutically acceptable salt or solvate thereof, wherein each R⁴ is —C(═O)N(R⁶)₂. In some embodiments, the invention provides a compound of Formula (IIIb), or a pharmaceutically acceptable salt or solvate thereof, wherein each R⁴ is —SO₂N(R⁶)₂.

In some embodiments, the invention provides a compound of Formula (IIIb), or a pharmaceutically acceptable salt or solvate thereof, wherein R¹ is —C₁-C₆alkyl, —C₁-C₆haloalkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (IIIb), or a pharmaceutically acceptable salt or solvate thereof, wherein R¹ is —C₁-C₆alkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (IIIb), or a pharmaceutically acceptable salt or solvate thereof, wherein R¹ is —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (IIIb), or a pharmaceutically acceptable salt or solvate thereof, wherein R¹ is —C(═O)OR⁶. In some embodiments, the invention provides a compound of Formula (IIIb), or a pharmaceutically acceptable salt or solvate thereof, wherein R¹ is —C(═O)OR⁶ and R⁶ is —C₁-C₆alkyl, —C₁-C₆alkylene-OR⁹, or —C₁-C₆alkylene-N(R⁹)₂. In some embodiments, the invention provides a compound of Formula (IIIb), or a pharmaceutically acceptable salt or solvate thereof, wherein R¹ is —C(═O)OR⁶. In some embodiments, the invention provides a compound of Formula (IIIb), or a pharmaceutically acceptable salt or solvate thereof, wherein R¹ is —C(═O)OR⁶ and R⁶ is —C₁-C₆alkyl. In some embodiments, the invention provides a compound of Formula (IIIb), or a pharmaceutically acceptable salt or solvate thereof, wherein R⁶ is methyl, ethyl, n-propyl, isobutyl, or t-butyl. In another embodiment, R⁶ is methyl. In yet a further embodiment, R⁶ is ethyl. In some embodiments, the invention provides a compound of Formula (IIIb), or a pharmaceutically acceptable salt or solvate thereof, wherein R¹ is —C(═O)OR⁶ and R⁶ is —C₁-C₆alkylene-OR⁹. In some embodiments, the invention provides a compound of Formula (IIIb), or a pharmaceutically acceptable salt or solvate thereof, wherein R¹ is —C(═O)OR⁶, R⁶ is —C₁-C₆alkylene-OR⁹, and R⁹ is —C₁-C₆alkyl. In a further embodiment, R⁶ is ethylene-OR⁹. In another embodiment, R⁶ is methylene-OR⁹. In another embodiment, R⁹ is selected from methyl, ethyl, n-propyl, or t-butyl. In one embodiment, R⁹ is methyl. In some embodiments, the invention provides a compound of Formula (IIIb), or a pharmaceutically acceptable salt or solvate thereof, wherein R¹ is —C(═O)OR⁶ and R⁶ is —C₁-C₆alkylene-N(R⁹)₂. In some embodiments, the invention provides a compound of Formula (IIIb), or a pharmaceutically acceptable salt or solvate thereof, wherein R¹ is —C(═O)N(R⁶)₂. In some embodiments, the invention provides a compound of Formula (IIIb), or a pharmaceutically acceptable salt or solvate thereof, wherein R¹ is —C(═O)N(R⁶)₂ and each R⁶ is independently selected from hydrogen and —C₁-C₆alkyl. In some embodiments, the invention provides a compound of Formula (IIIb), or a pharmaceutically acceptable salt or solvate thereof, wherein R¹ is —C(═O)N(R⁶)₂ and each R⁶ is independently selected from hydrogen and —C₁-C₆alkylene-OR⁹. In some embodiments, the invention provides a compound of Formula (IIIb), or a pharmaceutically acceptable salt or solvate thereof, wherein R¹ is —S(═O)₂R⁷. In some embodiments, the invention provides a compound of Formula (IIIb), or a pharmaceutically acceptable salt or solvate thereof, wherein R¹ is —S(═O)₂N(R⁶)₂. In some embodiments, the invention provides a compound of Formula (IIIb), or a pharmaceutically acceptable salt or solvate thereof, wherein R¹ is —C(═O)R⁷.

In some embodiments, the invention provides a compound of Formula (I), having the structure of Formula (IIIc):

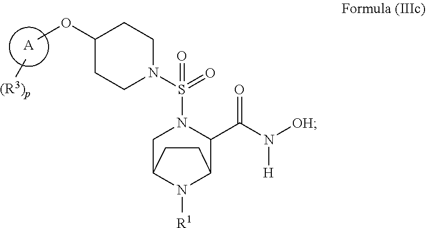

wherein:
\textcircled{A} is phenyl, C₃-C₁₀cycloalkyl, C₂-C₉heterocycloalkyl, or C₂-C₉heteroaryl;
R¹ is hydrogen, —C₁-C₆alkyl, —C₁-C₆haloalkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂;
each R³ is independently selected from halogen, —C₁-C₆alkyl, —C₁-C₆haloalkyl, —C₁-C₆alkylene-OR⁶, C₁-C₆haloalkoxy, —OR⁶, —N(R⁶)₂, —CN, —C(═O)R⁷, —C(═O)OR⁶, —C(═O)N(R⁶)₂, —NR⁶C(═O)R⁷, —NR⁶SO₂R⁷, —SO₂R⁷, —SO₂N(R⁶)₂, or phenyl optionally substituted with halogen;
each R⁶ is independently selected from hydrogen, —C₁-C₆alkyl, —C₁-C₆haloalkyl, —C₁-C₆alkylene-OR⁹, —C₁-C₆alkylene-N(R⁹)₂, and —C₁-C₆alkylene-C₂-C₉heterocycloalkyl;
or —N(R⁶)₂ represents C₃-C₅heterocycloalkyl;
each R⁷ is independently selected from —C₁-C₆alkyl, —C₁-C₆alkylene-OR⁹, —C₁-C₆alkylene-N(R⁹)₂, —C₂-C₉heterocycloalkyl, —C₂-C₉heteroaryl, —C₁-C₆alkylene-C₂-C₉heterocycloalkyl, and —C₁-C₆alkylene-C₂-C₉heteroaryl;
each R⁹ is independently selected from hydrogen and —C₁-C₆alkyl;
each occurrence of C₂-C₉heterocycloalkyl and C₂-C₉heteroaryl being optionally substituted with —C₁-C₆alkyl, halo-C₁-C₆alkyl or halogen; and
p is 0, 1, 2, or 3;
   or a pharmaceutically acceptable salt or solvate thereof.

In some embodiments, the invention provides a compound of Formula (IIIc), or a pharmaceutically acceptable salt or solvate thereof, wherein \textcircled{A} is phenyl. In some embodiments, the invention provides a compound of Formula (I), or a pharmaceutically acceptable salt or solvate thereof, wherein \textcircled{A} is C₃-C₁₀cycloalkyl or C₂-C₉heteroaryl. In some embodiments, the invention provides a compound of Formula some embodiments is a compound of Formula (IIIc), or a pharmaceutically acceptable salt or solvate thereof, wherein \textcircled{A} is C₂-C₉heteroaryl.

In some embodiments, the invention provides a compound of Formula (IIIc), or a pharmaceutically acceptable salt or solvate thereof, wherein Y is —O—. In some embodiments, the invention provides a compound of Formula (IIIc), or a pharmaceutically acceptable salt or solvate thereof, wherein Y is —CH₂O—. In some embodiments, the invention provides a compound of Formula (IIIc), or a pharmaceutically acceptable salt or solvate thereof, wherein Y is —OCH₂—. In some embodiments, the invention provides a compound of Formula (IIIc), or a pharmaceutically acceptable salt or solvate thereof, wherein Y is —N(R⁵)—. In some embodiments, the invention provides a compound of Formula (IIIc), or a pharmaceutically acceptable salt or solvate thereof, wherein Y is —N(H)—. In some embodiments, the invention provides a compound of Formula (IIIc), or a pharmaceutically acceptable salt or solvate thereof, wherein Y is —N(R⁵)— and R⁵ is —C₁-C₆alkyl. In some embodiments, the invention provides a compound of Formula (IIIc), or a pharmaceutically acceptable salt or solvate thereof, wherein Y is —N(R⁵) and R⁵ is methyl. In some embodiments, the invention provides a compound of Formula (IIIc), or a pharmaceutically acceptable salt or solvate thereof, wherein Y is —N(R⁵) and R⁵ is ethyl.

In some embodiments, the invention provides a compound of Formula (IIIc), or a pharmaceutically acceptable salt or solvate thereof, wherein p is 0. In some embodiments, the invention provides a compound of Formula (IIIc), or a pharmaceutically acceptable salt or solvate thereof, wherein p is 1. In some embodiments, the invention provides a compound of Formula (IIIc), or a pharmaceutically acceptable salt or solvate thereof, wherein p is 2.

In some embodiments, the invention provides a compound of Formula (IIIc), or a pharmaceutically acceptable salt or solvate thereof, wherein each R³ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂. In some embodiments, the invention provides a compound of Formula (IIIc), or a pharmaceutically acceptable salt or solvate thereof, wherein each R³ is independently selected from halogen, —C₁-C₆alkyl, —C₁-C₆haloalkyl, —OR⁶, and —C(═O)OR⁶. In some embodiments, the invention provides a compound of Formula (IIIc), or a pharmaceutically acceptable salt or solvate thereof, wherein each R³ is independently selected from halogen and —C₁-C₆alkyl. In some embodiments, the invention provides a compound of Formula (IIIc), or a pharmaceutically acceptable salt or solvate thereof, wherein each R³ is halogen. In some embodiments, the invention provides a compound of Formula (IIIc), or a pharmaceutically acceptable salt or solvate thereof, wherein each R³ is —C₁-C₆alkyl. In a further embodiment, R³ is methyl. In some embodiments, the invention provides a compound of Formula (IIIc), or a pharmaceutically acceptable salt or solvate thereof, wherein each R³ is —C₁-C₆haloalkyl. In a further embodiment, R³ is trifluoromethyl. In some embodiments, the invention provides a compound of Formula (IIIc), or a pharmaceutically acceptable salt or solvate thereof, wherein each R³ is independently selected from —OR⁶, —N(R⁶)₂, —C(═O)OR⁶, —C(═O)N(R⁶)₂, and —SO₂N(R⁶)₂. In some embodiments, the invention provides a compound of Formula (IIIc), or a pharmaceutically acceptable salt or solvate thereof, wherein each R³ is —OR⁶. In a further embodiment, R⁶ is hydrogen or methyl. In some embodiments, the invention provides a compound of Formula (IIIc), or a pharmaceutically acceptable salt or solvate thereof, wherein each R³ is —OR⁶ and each R⁶ is —C₁-C₆alkyl. In some embodiments, the invention provides a compound of Formula (IIIc), or a pharmaceutically acceptable salt or solvate thereof, wherein each R³ is —N(R⁶)₂. In some embodiments, the invention provides a compound of Formula (IIIc), or a pharmaceutically acceptable salt or solvate thereof, wherein each R³ is —C(═O)OR⁶. In some embodiments, the invention provides a compound of Formula (IIIc), or a pharmaceutically acceptable salt or solvate thereof, wherein each R³ is —C(═O)N(R⁶)₂. In some embodiments, the invention provides a compound of Formula (IIIc), or a pharmaceutically acceptable salt or solvate thereof, wherein each R³ is —SO₂N(R⁶)₂.

In some embodiments, the invention provides a compound of Formula (IIIc), or a pharmaceutically acceptable salt or solvate thereof, wherein R¹ is —C₁-C₆alkyl, —C₁-C₆haloalkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (IIIc), or a pharmaceutically acceptable salt or solvate thereof, wherein R¹ is —C₁-C₆alkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (IIIc), or a pharmaceutically acceptable salt or solvate thereof, wherein R¹ is —C(═O)R⁷, —C(═O)OR⁶, —C(═O)N(R⁶)₂, —S(═O)₂R⁷, or —S(═O)₂N(R⁶)₂. In some embodiments, the invention provides a compound of Formula (IIIc), or a pharmaceutically acceptable salt or solvate thereof, wherein R¹ is —C(═O)OR⁶. In some embodiments, the invention provides a compound of Formula (IIIc), or a pharmaceutically acceptable salt or solvate thereof, wherein R¹ is —C(═O)OR⁶ and R⁶ is —C₁-C₆alkyl, —C₁-C₆alkylene-OR⁹, or —C₁-C₆alkylene-N(R⁹)₂. In some embodiments, the invention provides a compound of Formula (IIIc), or a pharmaceutically acceptable salt or solvate thereof, wherein R¹ is —C(═O)OR⁶. In some embodiments, the invention provides a compound of Formula (IIIc), or a pharmaceutically acceptable salt or solvate thereof, wherein R¹ is —C(═O)OR⁶ and R⁶ is —C₁-C₆alkyl. In some embodiments, the invention provides a compound of Formula (IIIc), or a pharmaceutically acceptable salt or solvate thereof, wherein R⁶ is methyl, ethyl, n-propyl, isobutyl, or t-butyl. In another embodiment, R⁶ is methyl. In yet a further embodiment, R⁶ is ethyl. In some embodiments, the invention provides a compound of Formula (IIIc), or a pharmaceutically acceptable salt or solvate thereof, wherein R¹ is —C(═O)OR⁶ and R⁶ is —C₁-C₆alkylene-OR⁹. In some embodiments, the invention provides a compound of Formula (IIIc), or a pharmaceutically acceptable salt or solvate thereof, wherein R¹ is —C(═O)OR⁶, R⁶ is —C₁-C₆alkylene-OR⁹, and R⁹ is —C₁-C₆alkyl. In a further embodiment, R⁶ is ethylene-OR⁹. In another embodiment, R⁶ is methylene-OR⁹. In another embodiment, R⁹ is selected from methyl, ethyl, n-propyl, or t-butyl. In one embodiment, R⁹ is methyl. In some embodiments, the invention provides a compound of Formula (IIIc), or a pharmaceutically acceptable salt or solvate thereof, wherein R¹ is —C(═O)OR⁶ and R⁶ is —C₁-C₆alkylene-N(R⁹)₂. In some embodiments, the invention provides a compound of Formula (IIIc), or a pharmaceutically acceptable salt or solvate thereof, wherein R¹ is —C(═O)N(R⁶)₂. In some embodiments, the invention provides a compound of Formula (IIIc), or a pharmaceutically acceptable salt or solvate thereof, wherein R¹ is —C(═O)N(R⁶)₂ and each R⁶ is independently selected from hydrogen and —C₁-C₆alkyl. In some embodiments, the invention provides a compound of Formula (IIIc), or a pharmaceutically acceptable salt or solvate thereof, wherein R¹ is —C(═O)N(R⁶)₂ and each R⁶ is independently selected from hydrogen and —C₁-C₆alkylene-OR⁹. In some embodiments, the invention provides a compound of Formula (IIIc), or a pharmaceutically acceptable salt or solvate thereof, wherein R¹ is —S(═O)₂R⁷. In some embodiments, the invention provides a compound of Formula (IIIc), or a pharmaceutically acceptable salt or solvate thereof, wherein R¹ is —S(═O)₂N(R⁶)₂. In some embodiments, the invention provides a compound of Formula (IIIc), or a pharmaceutically acceptable salt or solvate thereof, wherein R¹ is —C(═O)R⁷.

In some embodiments described herein, the disclosed compound is a compound of Formula (I) or (Ia) or (Ib) or (Ic) or (II) or (III) or (IIIa) or (IIIb) or (IIIc), selected from the following compounds:

| Ex | Structure | Name |
|---|---|---|
| 1 | 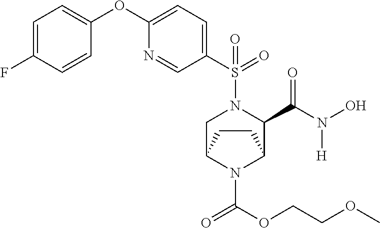 [SMILES: COCCOC(=O)N1[C@@H]2CC[C@H]1[C@H](C(=O)NO)N(S(=O)(=O)c1ccc(Oc3ccc(F)cc3)nc1)C2] | (1S,2R,5R)-2-methoxyethyl 3-((6-(4- fluorophenoxy)pyridin-3-yl)sulfonyl)-2- (hydroxycarbamoyl)-3,8- diazabicyclo[3.2.1]octane-8-carboxylate |
| 2 | 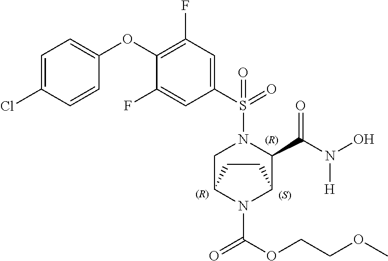 | (1S,2R,5R)-2-methoxyethyl 3-((4-(4- chlorophenoxy)-3,5- difluorophenyl)sulfonyl)-2- (hydroxycarbamoyl)-3,8- diazabicyclo[3.2.1]octane-8-carboxylate |
| 3 | 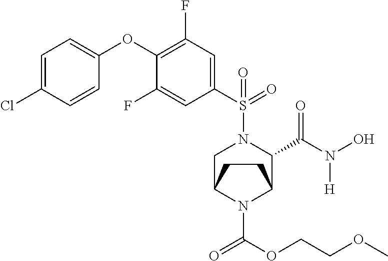 [SMILES: COCCOC(=O)N1[C@H]2CC[C@@H]1[C@@H](C(=O)NO)N(S(=O)(=O)c1cc(F)c(Oc3ccc(Cl)cc3)c(F)c1)C2] | 2-methoxyethyl (1R,2S,5S)-3-((4-(4- chlorophenoxy)-3,5- difluorophenyl)sulfonyl)-2- (hydroxycarbamoyl)-3,8- diazabicyclo[3.2.1]octane-8-carboxylate |
| 4 | 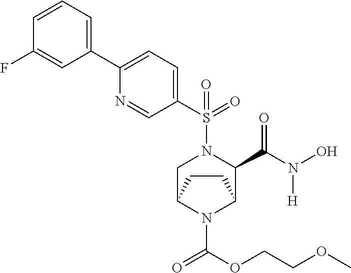 [SMILES: COCCOC(=O)N1[C@@H]2CC[C@H]1[C@H](C(=O)NO)N(S(=O)(=O)c1ccc(-c3cccc(F)c3)nc1)C2] | 2-methoxyethyl (1S,2R,5R)-3-((6-(3- fluorophenyl)pyridin-3-yl)sulfonyl)-2- (hydroxycarbamoyl)-3,8- diazabicyclo[3.2.1 ]octane-8-carboxylate |
| 5 | 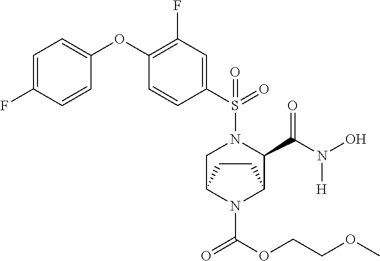 [SMILES: COCCOC(=O)N1[C@@H]2CC[C@H]1[C@H](C(=O)NO)N(S(=O)(=O)c1ccc(Oc3ccc(F)cc3)c(F)c1)C2] | 2-methoxyethyl (1S,2R,5R)-3-((3-fluoro- 4-(4-fluorophenoxy)phenyl)sulfonyl)-2- (hydroxycarbamoyl)-3,8- diazabicyclo[3.2.1]octane-8-carboxylate |
| 6 | 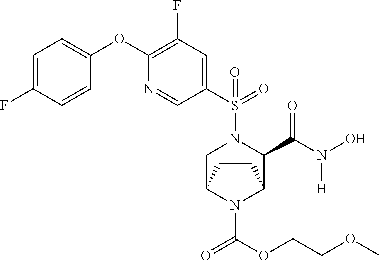 [SMILES: COCCOC(=O)N1[C@@H]2CC[C@H]1[C@H](C(=O)NO)N(S(=O)(=O)c1cnc(Oc3ccc(F)cc3)c(F)c1)C2] | 2-methoxyethyl (1S,2R,5R)-3-((5-fluoro- 6-(4-fluorophenoxy)pyridin-3- yl)sulfonyl)-2-(hydroxycarbamoyl)-3,8- diazabicyclo[3.2.1]octane-8-carboxylate |
| 7 | 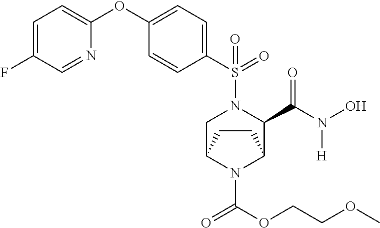 [SMILES: COCCOC(=O)N1[C@@H]2CC[C@H]1[C@H](C(=O)NO)N(S(=O)(=O)c1ccc(Oc3ccc(F)cn3)cc1)C2] | 2-methoxyethyl (1S,2R,5R)-3-((4-((5- fluoropyridin-2-yl)oxy)phenyl)sulfonyl)- 2-(hydroxycarbamoyl)-3,8- diazabicyclo[3.2.1]octane-8-carboxylate |
| 8 | 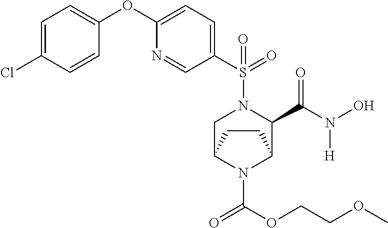 [SMILES: COCCOC(=O)N1[C@@H]2CC[C@H]1[C@H](C(=O)NO)N(S(=O)(=O)c1ccc(Oc3ccc(Cl)cc3)nc1)C2] | 2-methoxyethyl (1S,2R,5R)-3-((6-(4- chlorophenoxy)pyridin-3-yl)sulfonyl)-2- (hydroxycarbamoyl)-3,8- diazabicyclo[3.2.1]octane-8-carboxylate |
| 9 | 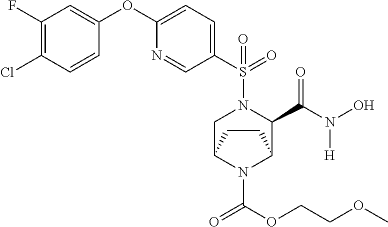 [SMILES: COCCOC(=O)N1[C@@H]2CC[C@H]1[C@H](C(=O)NO)N(S(=O)(=O)c1ccc(Oc3ccc(Cl)c(F)c3)nc1)C2] | 2-methoxyethyl (1S,2R,5R)-3-((6-(4- chloro-3-fluorophenoxy)pyridin-3- yl)sulfonyl)-2-(hydroxycarbamoyl)-3,8- diazabicyclo[3.2.1]octane-8-carboxylate |
| 10 | 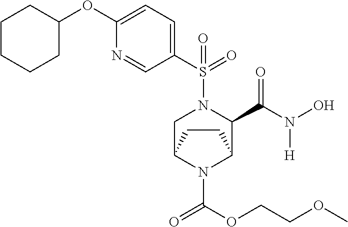 [SMILES: COCCOC(=O)N1[C@@H]2CC[C@H]1[C@H](C(=O)NO)N(S(=O)(=O)c1ccc(OC3CCCCC3)nc1)C2] | 2-methoxyethyl (1S,2R,5R)-3-((6- (cyclohexyloxy)pyridin-3-yl)sulfonyl)-2- (hydroxycarbamoyl)-3,8- diazabicyclo[3.2.1]octane-8-carboxylate |
| 11 | 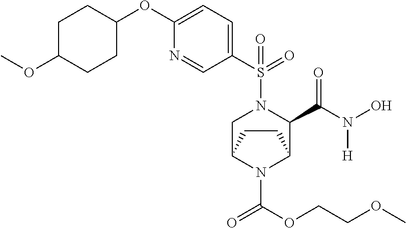 | 2-methoxyethyl (1S,2R,5R)-2- (hydroxycarbamoyl)-3-((6-((4- methoxycyclohexyl)oxy)pyridin-3- yl)sulfonyl)-3,8- diazabicyclo[3.2.1]octane-8-carboxylate |
| 12 | 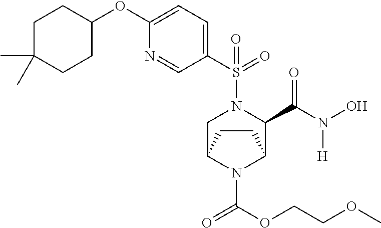 | 2-methoxyethyl (1S,2R,5R)-3-((6-((4,4- dimethylcyclohexyl)oxy)pyridin-3- yl)sulfonyl)-2-(hydroxycarbamoyl)-3,8- diazabicyclo[3.2.1]octane-8-carboxylate |
| 13 | 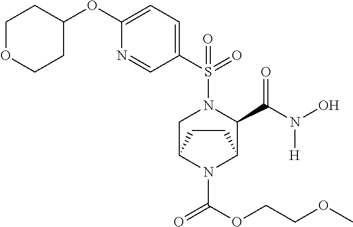 [SMILES: COCCOC(=O)N1[C@@H]2CC[C@H]1[C@H](C(=O)NO)N(S(=O)(=O)c1ccc(OC3CCOCC3)nc1)C2] | 2-methoxyethyl (1S,2R,5R)-2- (hydroxycarbamoyl)-3-((6-((tetrahydro- 2H-pyran-4-yl)oxy)pyridin-3-yl)sulfonyl)- 3,8-diazabicyclo[3.2.1]octane-8- carboxylate |
| 14 | 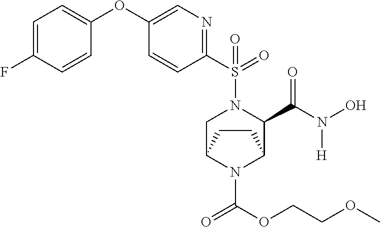 [SMILES: COCCOC(=O)N1[C@@H]2CC[C@H]1[C@H](C(=O)NO)N(S(=O)(=O)c1ccc(Oc3ccc(F)cc3)cn1)C2] | 2-methoxyethyl (1S,2R,5R)-3-((5-(4- fluorophenoxy)pyridin-2-yl)sulfonyl)-2- (hydroxycarbamoyl)-3,8- diazabicyclo[3.2.1]octane-8-carboxylate |
| 15 | 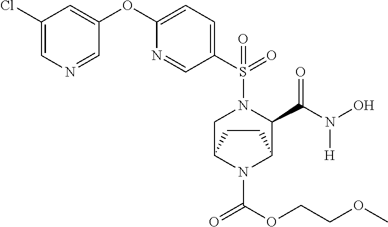 [SMILES: COCCOC(=O)N1[C@@H]2CC[C@H]1[C@H](C(=O)NO)N(S(=O)(=O)c1ccc(Oc3cncc(Cl)c3)nc1)C2] | (1S,2R,5R)-2-methoxyethyl 3-((6-((5- chloropyridin-3-yl)oxy)pyridin-3- yl)sulfonyl)-2-(hydroxycarbamoyl)-3,8- diazabicyclo[3.2.1]octane-8-carboxylate |
| 16 | 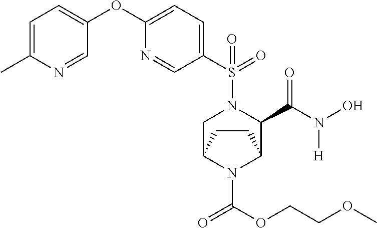 | 2-methoxyethyl (1S,2R,5R)-2- (hydroxycarbamoyl)-3-((6-((6- methylpyridin-3-yl)oxy)pyridin-3- yl)sulfonyl)-3,8- diazabicyclo[3.2.1]octane-8-carboxylate |
| 17 | 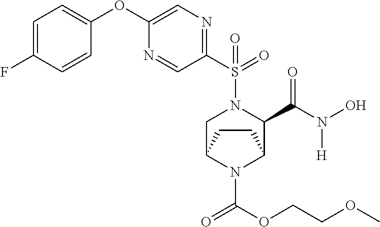 [SMILES: COCCOC(=O)N1[C@@H]2CC[C@H]1[C@H](C(=O)NO)N(S(=O)(=O)c1cnc(Oc3ccc(F)cc3)cn1)C2] | 2-methoxyethyl (1S,2R,5R)-3-((5-(4- fluorophenoxy)pyrazin-2-yl)sulfonyl)-2- (hydroxycarbamoyl)-3,8- diazabicyclo[3.2.1]octane-8-carboxylate |
| 18 | 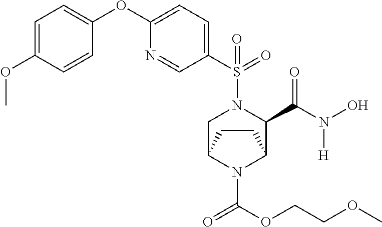 [SMILES: COCCOC(=O)N1[C@@H]2CC[C@H]1[C@H](C(=O)NO)N(S(=O)(=O)c1ccc(Oc3ccc(OC)cc3)nc1)C2] | 2-methoxyethyl (1S,2R,5R)-2- (hydroxycarbamoyl)-3-((6-(4- methoxyphenoxy)pyridin-3-yl)sulfonyl)- 3,8-diazabicyclo[3.2.1]octane-8- carboxylate |
| 19 | 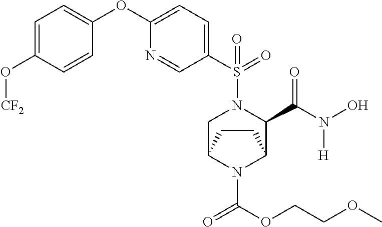 | 2-methoxyethyl (1S,2R,5R)-2- (hydroxycarbamoyl)-3-((6-(4- (trifluoromethoxy)phenoxy)pyridin-3- yl)sulfonyl)-3,8- diazabicyclo[3.2.1]octane-8-carboxylate |
| 20 | 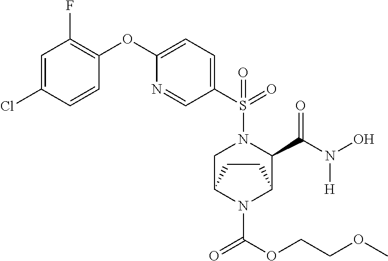 [SMILES: COCCOC(=O)N1[C@@H]2CC[C@H]1[C@H](C(=O)NO)N(S(=O)(=O)c1ccc(Oc3ccc(Cl)cc3F)nc1)C2] | 2-methoxyethyl (1S,2R,5R)-3-((6-(4- chloro-2-fluorophenoxy)pyridin-3- yl)sulfonyl)-2-(hydroxycarbamoyl)-3,8- diazabicyclo[3.2.1]octane-8-carboxylate |
| 21 | 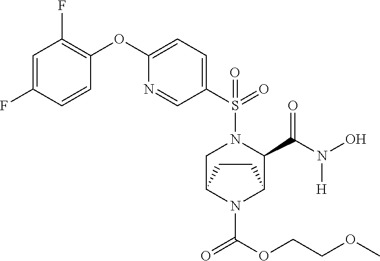 [SMILES: COCCOC(=O)N1[C@@H]2CC[C@H]1[C@H](C(=O)NO)N(S(=O)(=O)c1ccc(Oc3ccc(F)cc3F)nc1)C2] | 2-methoxyethyl (1S,2R,5R)-3-((6-(2,4- difluorophenoxy)pyridin-3-yl)sulfonyl)-2- (hydroxycarbamoyl)-3,8- diazabicyclo[3.2.1]octane-8-carboxylate |
| 22 | 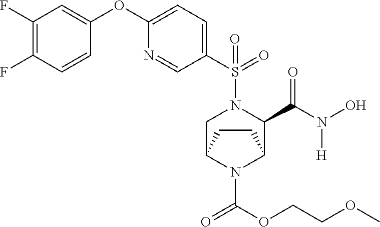 [SMILES: COCCOC(=O)N1[C@@H]2CC[C@H]1[C@H](C(=O)NO)N(S(=O)(=O)c1ccc(Oc3ccc(F)c(F)c3)nc1)C2] | 2-methoxyethyl (1S,2R,5R)-3-((6-(3,4- difluorophenoxy)pyridin-3-yl)sulfonyl)-2- (hydroxycarbamoyl)-3,8- diazabicyclo[3.2.1]octane-8-carboxylate |
| 23 | 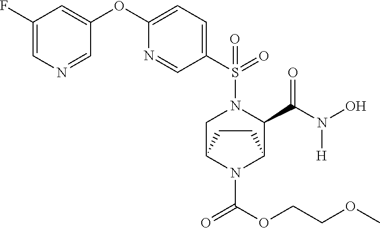 [SMILES: COCCOC(=O)N1[C@@H]2CC[C@H]1[C@H](C(=O)NO)N(S(=O)(=O)c1ccc(Oc3cncc(F)c3)nc1)C2] | 2-methoxyethyl (1S,2R,5R)-3-((6-((5- fluoropyridin-3-yl)oxy)pyridin-3- yl)sulfonyl)-2-(hydroxycarbamoyl)-3,8- diazabicyclo[3.2.1 ]octane-8-carboxylate |
| 24 | 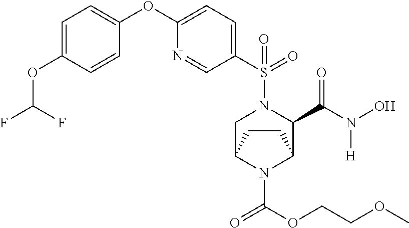 [SMILES: COCCOC(=O)N1[C@@H]2CC[C@H]1[C@H](C(=O)NO)N(S(=O)(=O)c1ccc(Oc3ccc(OC(F)F)cc3)nc1)C2] | 2-methoxyethyl (1S,2R,5R)-3-((6-(4- (difluoromethoxy)phenoxy)pyridin-3- yl)sulfonyl)-2-(hydroxycarbamoyl)-3,8- diazabicyclo[3.2.1]octane-8-carboxylate |
| 25 | 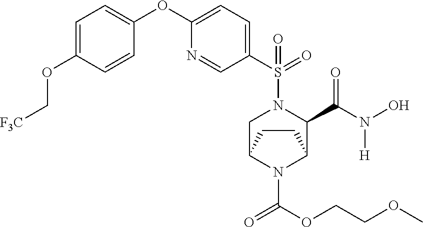 [SMILES: COCCOC(=O)N1[C@@H]2CC[C@H]1[C@H](C(=O)NO)N(S(=O)(=O)c1ccc(Oc3ccc(OCC(F)(F)F)cc3)nc1)C2] | 2-methoxyethyl (1S,2R,5R)-2- (hydroxycarbamoyl)-3-((6-(4-(2,2,2- trifluoroethoxy)phenoxy)pyridin-3- yl)sulfonyl)-3,8- diazabicyclo[3.2.1]octane-8-carboxylate |
| 26 | 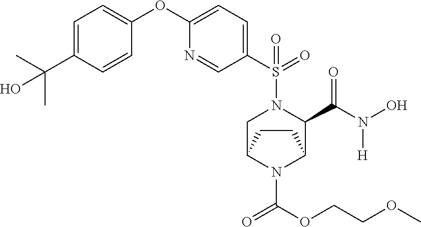 [SMILES: COCCOC(=O)N1[C@@H]2CC[C@H]1[C@H](C(=O)NO)N(S(=O)(=O)c1ccc(Oc3ccc(C(C)(C)O)cc3)nc1)C2] | 2-methoxyethyl (1S,2R,5R)-2- (hydroxycarbamoyl)-3-((6-(4-(2- hydroxypropan-2-yl)phenoxy)pyridin-3- yl)sulfonyl)-3,8- diazabicyclo[3.2.1]octane-8-carboxylate |
| 27 | 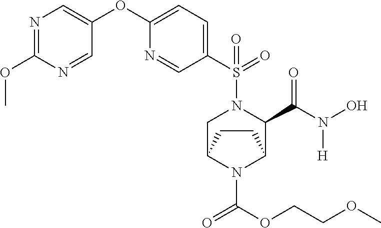 [SMILES: COCCOC(=O)N1[C@@H]2CC[C@H]1[C@H](C(=O)NO)N(S(=O)(=O)c1ccc(Oc3cnc(OC)nc3)nc1)C2] | 2-methoxyethyl (1S,2R,5R)-2- (hydroxycarbamoyl)-3-((6-((2- methoxypyrimidin-5-yl)oxy)pyridin-3- yl)sulfonyl)-3,8- diazabicyclo[3.2.1]octane-8-carboxylate |
| 28 | 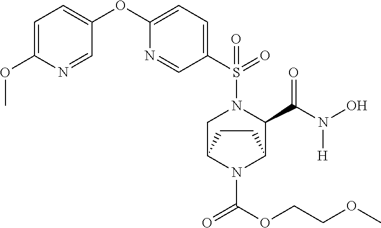 [SMILES: COCCOC(=O)N1[C@@H]2CC[C@H]1[C@H](C(=O)NO)N(S(=O)(=O)c1ccc(Oc3ccc(OC)nc3)nc1)C2] | 2-methoxyethyl (1S,2R,5R)-2- (hydroxycarbamoyl)-3-((6-((6- methoxypyridin-3-yl)oxy)pyridin-3- yl)sulfonyl)-3,8- diazabicyclo[3.2.1 ]octane-8-carboxylate |
| 29 | 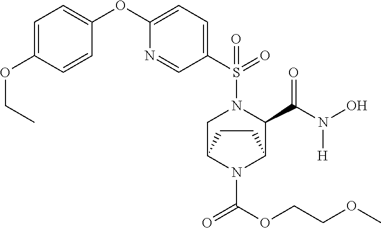 [SMILES: CCOc1ccc(Oc2ccc(S(=O)(=O)N3C[C@H]4CC[C@@H]([C@@H]3C(=O)NO)N4C(=O)OCCOC)cn2)cc1] | 2-methoxyethyl (1S,2R,5R)-3-((6-(4- ethoxyphenoxy)pyridin-3-yl)sulfonyl)-2- (hydroxycarbamoyl)-3,8- diazabicyclo[3.2.1]octane-8-carboxylate |
| 30 | 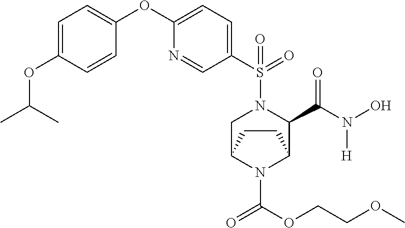 [SMILES: COCCOC(=O)N1[C@@H]2CC[C@H]1[C@H](C(=O)NO)N(S(=O)(=O)c1ccc(Oc3ccc(OC(C)C)cc3)nc1)C2] | 2-methoxyethyl (1S,2R,5R)-2- (hydroxycarbamoyl)-3-((6-(4- isopropoxyphenoxy)pyridin-3- yl)sulfonyl)-3,8- diazabicyclo[3.2.1]octane-8-carboxylate |
| 31 | 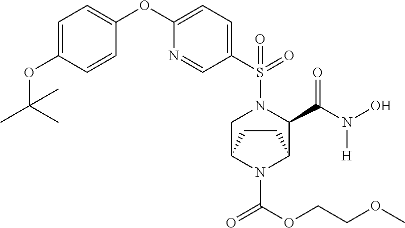 [SMILES: COCCOC(=O)N1[C@@H]2CC[C@H]1[C@H](C(=O)NO)N(S(=O)(=O)c1ccc(Oc3ccc(OC(C)(C)C)cc3)nc1)C2] | 2-methoxyethyl (1S,2R,5R)-3-((6-(4-(tert- butoxy)phenoxy)pyridin-3-yl)sulfonyl)-2- (hydroxycarbamoyl)-3,8- diazabicyclo[3.2.1 ]octane-8-carboxylate |
| 32 | 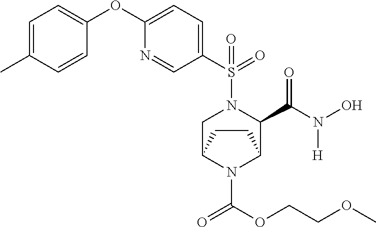 | 2-methoxyethyl (1S,2R,5R)-2- (hydroxycarbamoyl)-3-((6-(p- tolyloxy)pyridin-3-yl)sulfonyl)-3,8- diazabicyclo[3.2.1]octane-8-carboxylate |
| 33 | 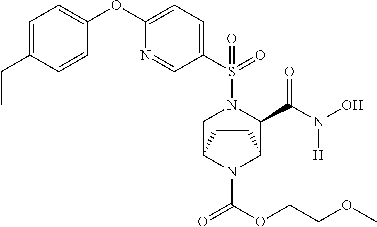 [SMILES: CCc1ccc(Oc2ccc(S(=O)(=O)N3C[C@H]4CC[C@@H]([C@@H]3C(=O)NO)N4C(=O)OCCOC)cn2)cc1] | 2-methoxyethyl (1S,2R,5R)-3-((6-(4- ethylphenoxy)pyridin-3-yl)sulfonyl)-2- (hydroxycarbamoyl)-3,8- diazabicyclo[3.2.1]octane-8-carboxylate |
| 34 | 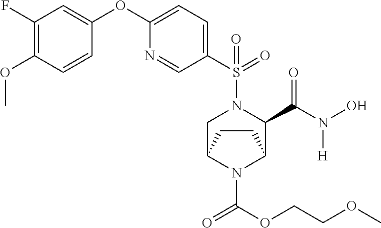 [SMILES: COCCOC(=O)N1[C@@H]2CC[C@H]1[C@H](C(=O)NO)N(S(=O)(=O)c1ccc(Oc3ccc(OC)c(F)c3)nc1)C2] | 2-methoxyethyl (1S,2R,5R)-3-((6-(3- fluoro-4-methoxyphenoxy)pyridin-3- yl)sulfonyl)-2-(hydroxycarbamoyl)-3,8- diazabicyclo[3.2.1]octane-8-carboxylate |
| 35 | 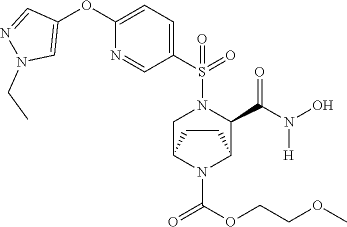 [SMILES: CCn1cc(Oc2ccc(S(=O)(=O)N3C[C@H]4CC[C@@H]([C@@H]3C(=O)NO)N4C(=O)OCCOC)cn2)cn1] | 2-methoxyethyl (1S,2R,5R)-3-((6-((1- ethyl-1H-pyrazol-4-yl)oxy)pyridin-3- yl)sulfonyl)-2-(hydroxycarbamoyl)-3,8- diazabicyclo[3.2.1]octane-8-carboxylate |
| 36 | 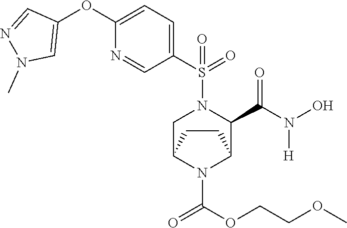 [SMILES: COCCOC(=O)N1[C@@H]2CC[C@H]1[C@H](C(=O)NO)N(S(=O)(=O)c1ccc(Oc3cnn(C)c3)nc1)C2] | 2-methoxyethyl (1S,2R,5R)-2- (hydroxycarbamoyl)-3-((6-((1-methyl-1H- pyrazol-4-yl)oxy)pyridin-3-yl)sulfonyl)- 3,8-diazabicyclo[3.2.1]octane-8- carboxylate |
| 37 | 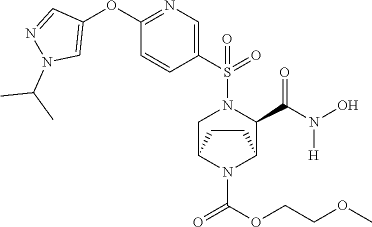 [SMILES: COCCOC(=O)N1[C@@H]2CC[C@H]1[C@H](C(=O)NO)N(S(=O)(=O)c1ccc(Oc3cnn(C(C)C)c3)nc1)C2] | 2-methoxyethyl (1S,2R,5R)-2- (hydroxycarbamoyl)-3-((6-((1-isopropyl- 1H-pyrazol-4-yl)oxy)pyridin-3- yl)sulfonyl)-3,8- diazabicyclo[3.2.1]octane-8-carboxylate |
| 38 | 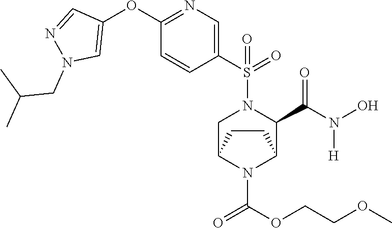 [SMILES: COCCOC(=O)N1[C@@H]2CC[C@H]1[C@H](C(=O)NO)N(S(=O)(=O)c1ccc(Oc3cnn(CC(C)C)c3)nc1)C2] | 2-methoxyethyl (1S,2R,5R)-2- (hydroxycarbamoyl)-3-((6-((1-isobutyl- 1H-pyrazol-4-yl)oxy)pyridin-3- yl)sulfonyl)-3,8- diazabicyclo[3.2.1]octane-8-carboxylate |
| 39 | 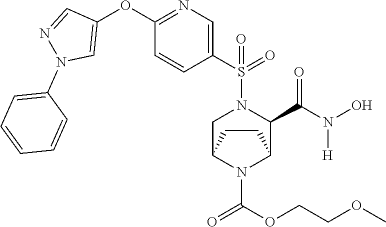 [SMILES: COCCOC(=O)N1[C@@H]2CC[C@H]1[C@H](C(=O)NO)N(S(=O)(=O)c1ccc(Oc3cnn(-c4ccccc4)c3)nc1)C2] | 2-methoxyethyl (1S,2R,5R)-2- (hydroxycarbamoyl)-3-((6-((1-phenyl-1H- pyrazol-4-yl)oxy)pyridin-3-yl)sulfonyl)- 3,8-diazabicyclo[3.2.1]octane-8- carboxylate |
| 40 | 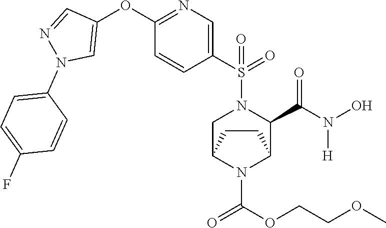 [SMILES: COCCOC(=O)N1[C@@H]2CC[C@H]1[C@H](C(=O)NO)N(S(=O)(=O)c1ccc(Oc3cnn(-c4ccc(F)cc4)c3)nc1)C2] | 2-methoxyethyl (1S,2R,5R)-3-((6-((1-(4- fluorophenyl)-1H-pyrazol-4- yl)oxy)pyridin-3-yl)sulfonyl)-2- (hydroxycarbamoyl)-3,8- diazabicyclo[3.2.1 ]octane-8-carboxylate |
| 41 | 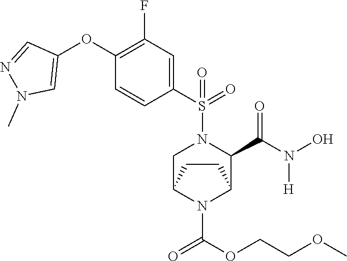 [SMILES: COCCOC(=O)N1[C@@H]2CC[C@H]1[C@H](C(=O)NO)N(S(=O)(=O)c1ccc(Oc3cnn(C)c3)c(F)c1)C2] | 2-methoxyethyl (1S,2R,5R)-3-((3-fluoro- 4-((1-methyl-1H-pyrazol-4- yl)oxy)phenyl)sulfonyl)-2- (hydroxycarbamoyl)-3,8- diazabicyclo[3.2.1]octane-8-carboxylate |
| 42 | 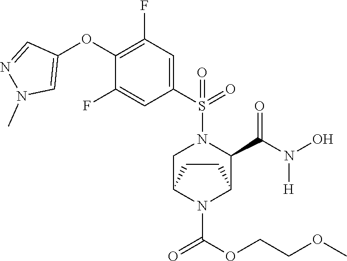 [SMILES: COCCOC(=O)N1[C@@H]2CC[C@H]1[C@H](C(=O)NO)N(S(=O)(=O)c1cc(F)c(Oc3cnn(C)c3)c(F)c1)C2] | 2-methoxyethyl (1S,2R,5R)-3-((3,5- difluoro-4-((1-methyl-1H-pyrazol-4- yl)oxy)phenyl)sulfonyl)-2- (hydroxycarbamoyl)-3,8- diazabicyclo[3.2.1 ]octane-8-carboxylate |
| 43 | 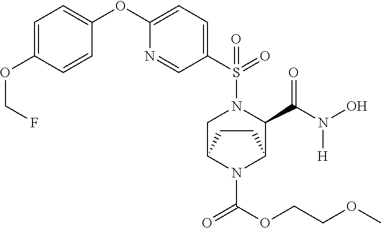 [SMILES: COCCOC(=O)N1[C@@H]2CC[C@H]1[C@H](C(=O)NO)N(S(=O)(=O)c1ccc(Oc3ccc(OCF)cc3)nc1)C2] | 2-methoxyethyl (1S,2R,5R)-3-((6-(4- (fluoromethoxy)phenoxy)pyridin-3- yl)sulfonyl)-2-(hydroxycarbamoyl)-3,8- diazabicyclo[3.2.1]octane-8-carboxylate |
| 44 | 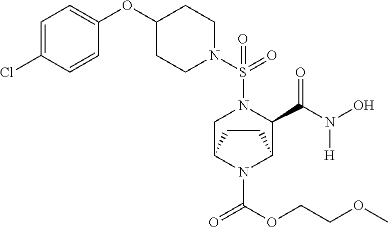 [SMILES: COCCOC(=O)N1[C@@H]2CC[C@H]1[C@H](C(=O)NO)N(S(=O)(=O)N1CCC(Oc3ccc(Cl)cc3)CC1)C2] | (1S,2R,5R)-2-methoxyethyl 3-((4-(4- chlorophenoxy)piperidin-1-yl)sulfonyl)-2- (hydroxycarbamoyl)-3,8- diazabicyclo[3.2.1]octane-8-carboxylate |
| 45 | 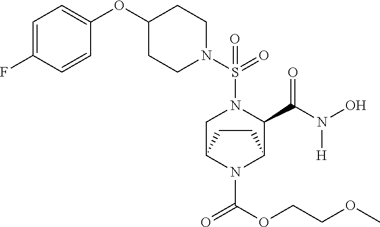 [SMILES: COCCOC(=O)N1[C@@H]2CC[C@H]1[C@H](C(=O)NO)N(S(=O)(=O)N1CCC(Oc3ccc(F)cc3)CC1)C2] | 2-methoxyethyl (1S,2R,5R)-3-((4-(4- fluorophenoxy)piperidin-1-yl)sulfonyl)-2- (hydroxycarbamoyl)-3,8- diazabicyclo[3.2.1]octane-8-carboxylate |
| 46 | 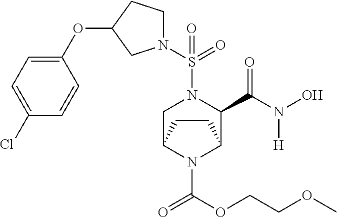 [SMILES: COCCOC(=O)N1[C@@H]2CC[C@H]1[C@H](C(=O)NO)N(S(=O)(=O)N1CCC(Oc3ccc(Cl)cc3)C1)C2] | 2-methoxyethyl (1S,2R,5R)-3-((3-(4- chlorophenoxy)pyrrolidin-1-yl)sulfonyl)- 2-(hydroxycarbamoyl)-3,8- diazabicyclo[3.2.1]octane-8-carboxylate |
| 47 | 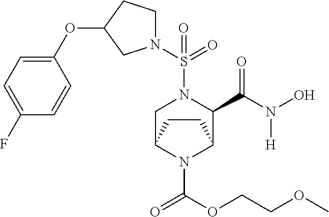 [SMILES: COCCOC(=O)N1[C@@H]2CC[C@H]1[C@H](C(=O)NO)N(S(=O)(=O)N1CCC(Oc3ccc(F)cc3)C1)C2] | 2-methoxyethyl (1S,2R,5R)-3-((3-(4- fluorophenoxy)pyrrolidin-1-yl)sulfonyl)- 2-(hydroxycarbamoyl)-3,8- diazabicyclo[3.2.1]octane-8-carboxylate |
| 48 | 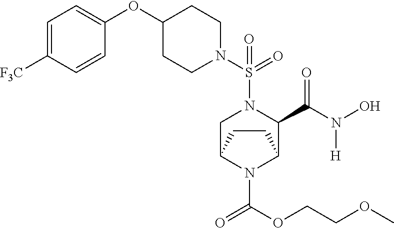 [SMILES: COCCOC(=O)N1[C@@H]2CC[C@H]1[C@H](C(=O)NO)N(S(=O)(=O)N1CCC(Oc3ccc(C(F)(F)F)cc3)CC1)C2] | 2-methoxyethyl (1S,2R,5R)-2- (hydroxycarbamoyl)-3-((4-(4- (trifluoromethyl)phenoxy)piperidin-1- yl)sulfonyl)-3,8- diazabicyclo[3.2.1]octane-8-carboxylate |
| 49 | 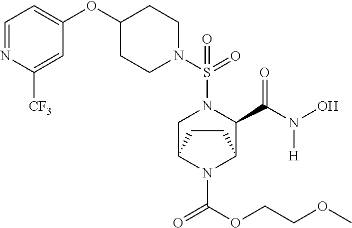 [SMILES: COCCOC(=O)N1[C@@H]2CC[C@H]1[C@H](C(=O)NO)N(S(=O)(=O)N1CCC(Oc3ccnc(C(F)(F)F)c3)CC1)C2] | 2-methoxyethyl (1S,2R,5R)-2- (hydroxycarbamoyl)-3-((4-((2- (trifluoromethyl)pyridin-4- yl)oxy)piperidin-1-yl)sulfonyl)-3,8- diazabicyclo[3.2.1]octane-8-carboxylate |
| 50 | 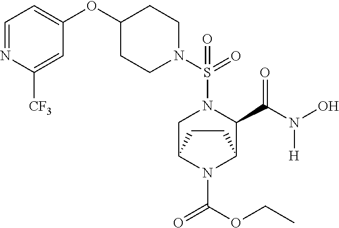 [SMILES: CCOC(=O)N1[C@@H]2CC[C@H]1[C@H](C(=O)NO)N(S(=O)(=O)N1CCC(Oc3ccnc(C(F)(F)F)c3)CC1)C2] | ethyl (1S,2R,5R)-2-(hydroxycarbamoyl)- 3-((4-((2-(trifluoromethyl)pyridin-4- yl)oxy)piperidin-1-yl)sulfonyl)-3,8- diazabicyclo[3.2.1]octane-8-carboxylate |
| 51 | 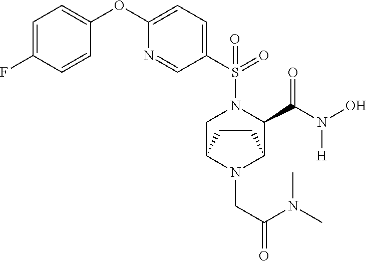 [SMILES: CN(C)C(=O)CN1[C@@H]2CC[C@H]1[C@H](C(=O)NO)N(S(=O)(=O)c1ccc(Oc3ccc(F)cc3)nc1)C2] | (1S,2R,5R)-8-(2-(dimethylamino)-2- oxoethyl)-3-((6-(4-fluorophenoxy)pyridin- 3-yl)sulfonyl)-N-hydroxy-3,8- diazabicyclo[3.2.1 ]octane-2-carboxamide |
| 52 | 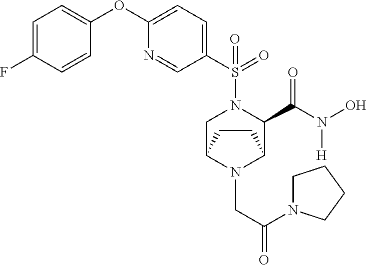 [SMILES: O=C(NO)[C@H]1[C@@H]2CC[C@H](CN1S(=O)(=O)c1ccc(Oc3ccc(F)cc3)nc1)N2CC(=O)N1CCCC1] | (1S,2R,5R)-3-((6-(4- fluorophenoxy)pyridin-3-yl)sulfonyl)-N- hydroxy-8-(2-oxo-2-(pyrrolidin-1- yl)ethyl)-3,8-diazabicyclo[3.2.1]octane-2- carboxamide |
| 53 | 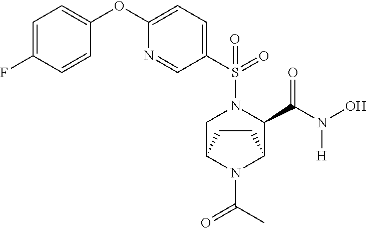 [SMILES: CC(=O)N1[C@@H]2CC[C@H]1[C@H](C(=O)NO)N(S(=O)(=O)c1ccc(Oc3ccc(F)cc3)nc1)C2] | (1S,2R,5R)-8-acetyl-3-((6-(4- fluorophenoxy)pyridin-3-yl)sulfonyl)-N- hydroxy-3,8-diazabicyclo[3.2.1]octane-2- carboxamide |
| 54 | 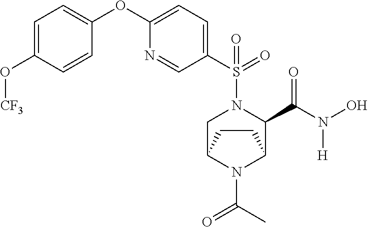 [SMILES: CC(=O)N1[C@@H]2CC[C@H]1[C@H](C(=O)NO)N(S(=O)(=O)c1ccc(Oc3ccc(OC(F)(F)F)cc3)nc1)C2] | (1S,2R,5R)-8-acetyl-N-hydroxy-3-((6-(4- (trifluoromethoxy)phenoxy)pyridin-3- yl)sulfonyl)-3,8- diazabicyclo[3.2.1]octane-2-carboxamide |
| 55 | 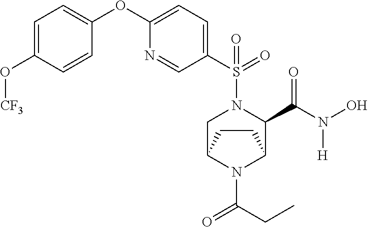 [SMILES: CCC(=O)N1[C@@H]2CC[C@H]1[C@H](C(=O)NO)N(S(=O)(=O)c1ccc(Oc3ccc(OC(F)(F)F)cc3)nc1)C2] | (1S,2R,5R)-N-hydroxy-8-propionyl-3-((6- (4-(trifluoromethoxy)phenoxy)pyridin-3- yl)sulfonyl)-3,8- diazabicyclo[3.2.1]octane-2-carboxamide |
| 56 | 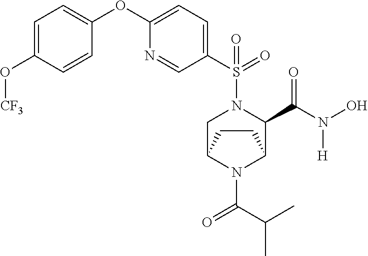 [SMILES: CC(C)C(=O)N1[C@@H]2CC[C@H]1[C@H](C(=O)NO)N(S(=O)(=O)c1ccc(Oc3ccc(OC(F)(F)F)cc3)nc1)C2] | (1S,2R,5R)-N-hydroxy-8-isobutyryl-3-((6- (4-(trifluoromethoxy)phenoxy)pyridin-3- yl)sulfonyl)-3,8- diazabicyclo[3.2.1]octane-2-carboxamide |
| 57 | 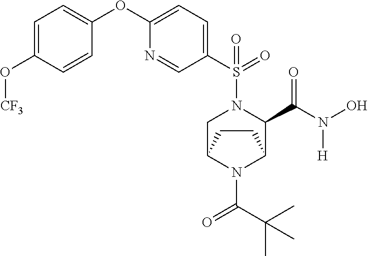 [SMILES: CC(C)(C)C(=O)N1[C@@H]2CC[C@H]1[C@H](C(=O)NO)N(S(=O)(=O)c1ccc(Oc3ccc(OC(F)(F)F)cc3)nc1)C2] | (1S,2R,5R)-N-hydroxy-8-pivaloyl-3-((6- (4-(trifluoromethoxy)phenoxy)pyridin-3- yl)sulfonyl)-3,8- diazabicyclo[3.2.1]octane-2-carboxamide |
| 58 | 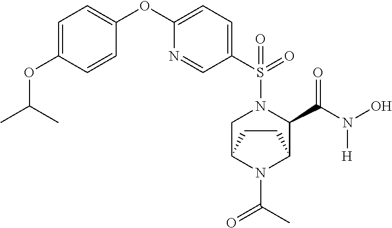 [SMILES: CC(=O)N1[C@@H]2CC[C@H]1[C@H](C(=O)NO)N(S(=O)(=O)c1ccc(Oc3ccc(OC(C)C)cc3)nc1)C2] | (1S,2R,5R)-8-acetyl-N-hydroxy-3-((6-(4- isopropoxyphenoxy)pyridin-3- yl)sulfonyl)-3,8- diazabicyclo[3.2.1]octane-2-carboxamide |
| 59 | 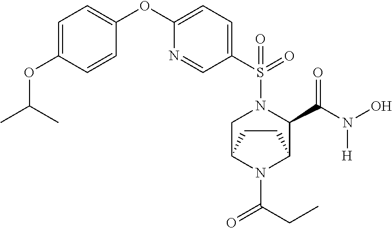 [SMILES: CCC(=O)N1[C@@H]2CC[C@H]1[C@H](C(=O)NO)N(S(=O)(=O)c1ccc(Oc3ccc(OC(C)C)cc3)nc1)C2] | (1S,2R,5R)-N-hydroxy-3-((6-(4- isopropoxyphenoxy)pyridin-3- yl)sulfonyl)-8-propionyl-3,8- diazabicyclo[3.2.1]octane-2-carboxamide |
| 60 | 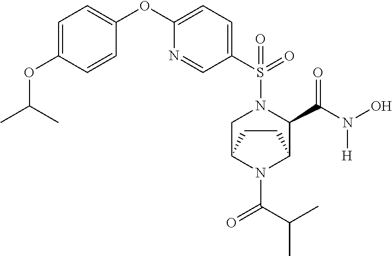 [SMILES: CC(C)Oc1ccc(Oc2ccc(S(=O)(=O)N3C[C@H]4CC[C@@H]([C@@H]3C(=O)NO)N4C(=O)C(C)C)cn2)cc1] | (1S,2R,5R)-N-hydroxy-8-isobutyryl-3-((6- (4-isopropoxyphenoxy)pyridin-3- yl)sulfonyl)-3,8- diazabicyclo[3.2.1]octane-2-carboxamide |
| 61 | 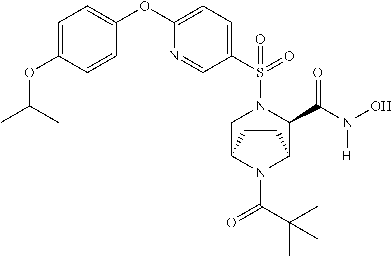 [SMILES: CC(C)Oc1ccc(Oc2ccc(S(=O)(=O)N3C[C@H]4CC[C@@H]([C@@H]3C(=O)NO)N4C(=O)C(C)(C)C)cn2)cc1] | (1S,2R,5R)-N-hydroxy-3-((6-(4- isopropoxyphenoxy)pyridin-3- yl)sulfonyl)-8-pivaloyl-3,8- diazabicyclo[3.2.1]octane-2-carboxamide |
| 62 | 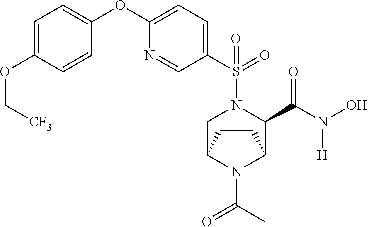 [SMILES: CC(=O)N1[C@@H]2CC[C@H]1[C@H](C(=O)NO)N(S(=O)(=O)c1ccc(Oc3ccc(OCC(F)(F)F)cc3)nc1)C2] | (1S,2R,5R)-8-acetyl-N-hydroxy-3-((6-(4- (2,2,2-trifluoroethoxy)phenoxy)pyridin-3- yl)sulfonyl)-3,8- diazabicyclo[3.2.1]octane-2-carboxamide |
| 63 | 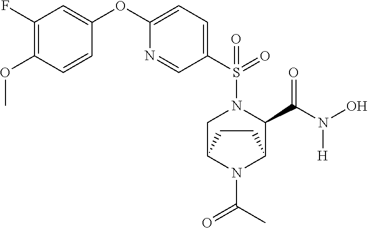 [SMILES: COc1ccc(Oc2ccc(S(=O)(=O)N3C[C@H]4CC[C@@H]([C@@H]3C(=O)NO)N4C(C)=O)cn2)cc1F] | (1S,2R,5R)-8-acetyl-3-((6-(3-fluoro-4- methoxyphenoxy)pyridin-3-yl)sulfonyl)- N-hydroxy-3,8-diazabicyclo[3.2.1]octane- 2-carboxamide |
| 64 | 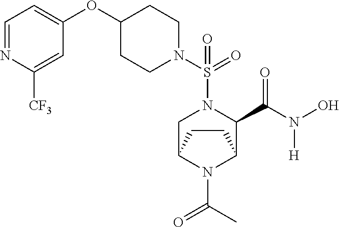 [SMILES: CC(=O)N1[C@@H]2CC[C@H]1[C@H](C(=O)NO)N(S(=O)(=O)N1CCC(Oc3ccnc(C(F)(F)F)c3)CC1)C2] | (1S,2R,5R)-8-acetyl-N-hydroxy-3-((4-((2- (trifluoromethyl)pyridin-4- yl)oxy)piperidin-1-yl)sulfonyl)-3,8- diazabicyclo[3.2.1]octane-2-carboxamide |
| 65 | 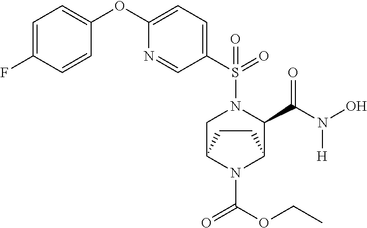 [SMILES: CCOC(=O)N1[C@@H]2CC[C@H]1[C@H](C(=O)NO)N(S(=O)(=O)c1ccc(Oc3ccc(F)cc3)nc1)C2] | ethyl (1S,2R,5R)-3-((6-(4- fluorophenoxy)pyridin-3-yl)sulfonyl)-2- (hydroxycarbamoyl)-3,8- diazabicyclo[3.2.1]octane-8-carboxylate |
| 66 | 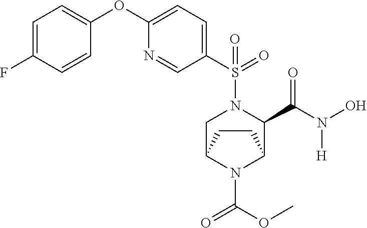 [SMILES: COC(=O)N1[C@@H]2CC[C@H]1[C@H](C(=O)NO)N(S(=O)(=O)c1ccc(Oc3ccc(F)cc3)nc1)C2] | methyl (1S,2R,5R)-3-((6-(4- fluorophenoxy)pyridin-3-yl)sulfonyl)-2- (hydroxycarbamoyl)-3,8- diazabicyclo[3.2.1]octane-8-carboxylate |
| 67 | 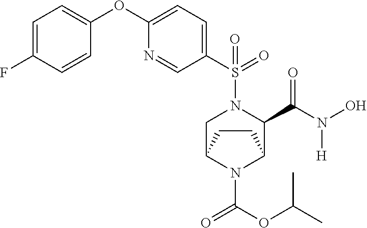 [SMILES: CC(C)OC(=O)N1[C@@H]2CC[C@H]1[C@H](C(=O)NO)N(S(=O)(=O)c1ccc(Oc3ccc(F)cc3)nc1)C2] | isopropyl (1S,2R,5R)-3-((6-(4- fluorophenoxy)pyridin-3-yl)sulfonyl)-2- (hydroxycarbamoyl)-3,8- diazabicyclo[3.2.1]octane-8-carboxylate |
| 68 | 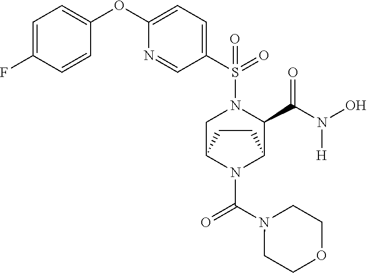 [SMILES: O=C(NO)[C@H]1[C@@H]2CC[C@H](CN1S(=O)(=O)c1ccc(Oc3ccc(F)cc3)nc1)N2C(=O)N1CCOCC1] | (1S,2R,5R)-3-((6-(4- fluorophenoxy)pyridin-3-yl)sulfonyl)-N- hydroxy-8-(morpholine-4-carbonyl)-3,8- diazabicyclo[3.2.1]octane-2-carboxamide |
| 69 | 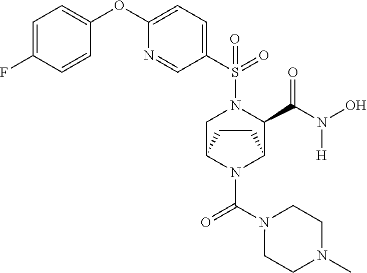 [SMILES: CN1CCN(C(=O)N2[C@@H]3CC[C@H]2[C@H](C(=O)NO)N(S(=O)(=O)c2ccc(Oc4ccc(F)cc4)nc2)C3)CC1] | (1S,2R,5R)-3-((6-(4- fluorophenoxy)pyridin-3-yl)sulfonyl)-N- hydroxy-8-(4-methylpiperazine-1- carbonyl)-3,8-diazabicyclo[3.2.1]octane-2- carboxamide |
| 70 | 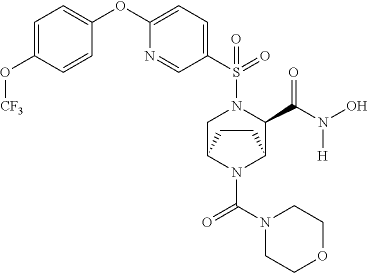 [SMILES: O=C(NO)[C@H]1[C@@H]2CC[C@H](CN1S(=O)(=O)c1ccc(Oc3ccc(OC(F)(F)F)cc3)nc1)N2C(=O)N1CCOCC1] | (1S,2R,5R)-N-hydroxy-8-(morpholine-4- carbonyl)-3-((6-(4- (trifluoromethoxy)phenoxy)pyridin-3- yl)sulfonyl)-3,8- diazabicyclo[3.2.1]octane-2-carboxamide |
| 71 | 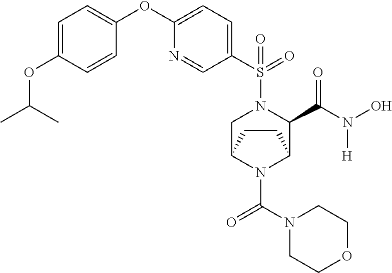 [SMILES: CC(C)Oc1ccc(Oc2ccc(S(=O)(=O)N3C[C@H]4CC[C@@H]([C@@H]3C(=O)NO)N4C(=O)N3CCOCC3)cn2)cc1] | (1S,2R,5R)-N-hydroxy-3-((6-(4- isopropoxyphenoxy)pyridin-3- yl)sulfonyl)-8-(morpholine-4-carbonyl)- 3,8-diazabicyclo[3.2.1 ]octane-2- carboxamide |
| 72 | 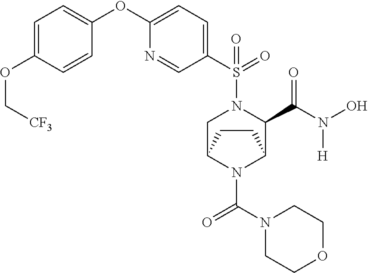 [SMILES: O=C(NO)[C@H]1[C@@H]2CC[C@H](CN1S(=O)(=O)c1ccc(Oc3ccc(OCC(F)(F)F)cc3)nc1)N2C(=O)N1CCOCC1] | (1S,2R,5R)-N-hydroxy-8-(morpholine-4- carbonyl)-3-((6-(4-(2,2,2- trifluoroethoxy)phenoxy)pyridin-3- yl)sulfonyl)-3,8- diazabicyclo[3.2.1]octane-2-carboxamide |
| 73 | 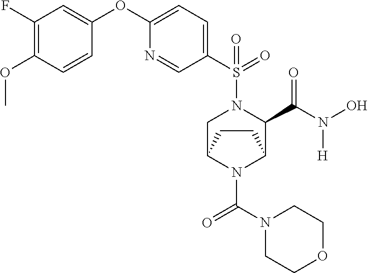 [SMILES: COc1ccc(Oc2ccc(S(=O)(=O)N3C[C@H]4CC[C@@H]([C@@H]3C(=O)NO)N4C(=O)N3CCOCC3)cn2)cc1F] | (1S,2R,5R)-3-((6-(3-fluoro-4- methoxyphenoxy)pyridin-3-yl)sulfonyl)- N-hydroxy-8-(morpholine-4-carbonyl)- 3,8-diazabicyclo[3.2.1]octane-2- carboxamide |
| 74 | 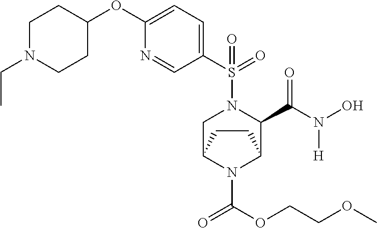 [SMILES: CCN1CCC(Oc2ccc(S(=O)(=O)N3C[C@H]4CC[C@@H]([C@@H]3C(=O)NO)N4C(=O)OCCOC)cn2)CC1] | 2-methoxyethyl (1S,2R,5R)-3-((6-((1- ethylpiperidin-4-yl)oxy)pyridin-3- yl)sulfonyl)-2-(hydroxycarbamoyl)-3,8- diazabicyclo[3.2.1]octane-8-carboxylate |
| 75 | 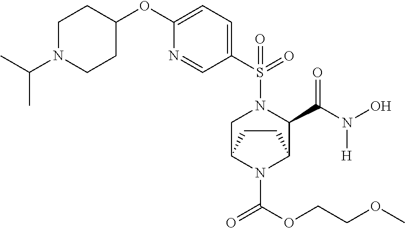 [SMILES: COCCOC(=O)N1[C@@H]2CC[C@H]1[C@H](C(=O)NO)N(S(=O)(=O)c1ccc(OC3CCN(C(C)C)CC3)nc1)C2] | 2-methoxyethyl (1S,2R,5R)-2- (hydroxycarbamoyl)-3-((6-((1- isopropylpiperidin-4-yl)oxy)pyridin-3- yl)sulfonyl)-3,8- diazabicyclo[3.2.1]octane-8-carboxylate |
| 76 | 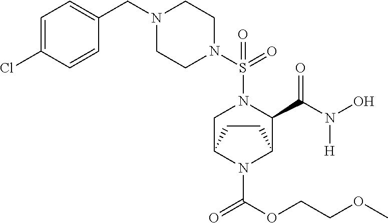 [SMILES: COCCOC(=O)N1[C@@H]2CC[C@H]1[C@H](C(=O)NO)N(S(=O)(=O)N1CCN(Cc3ccc(Cl)cc3)CC1)C2] | 2-methoxyethyl (1S,2R,5R)-3-((4-(4- chlorobenzyl)piperazin-1-yl)sulfonyl)-2- (hydroxycarbamoyl)-3,8- diazabicyclo[3.2.1]octane-8-carboxylate |
| 77 | 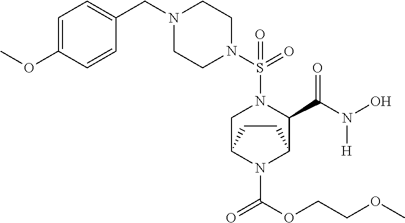 | 2-methoxyethyl (1S,2R,5R)-2- (hydroxycarbamoyl)-3-((4-(4- methoxybenzyl)piperazin-1-yl)sulfonyl)- 3,8-diazabicyclo[3.2.1]octane-8- carboxylate |
| 78 | 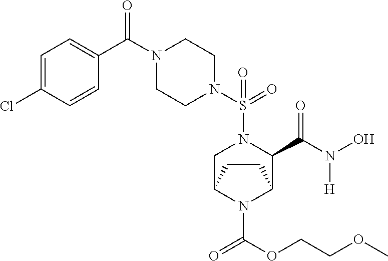 [SMILES: COCCOC(=O)N1[C@@H]2CC[C@H]1[C@H](C(=O)NO)N(S(=O)(=O)N1CCN(C(=O)c3ccc(Cl)cc3)CC1)C2] | 2-methoxyethyl (1S,2R,5R)-3-((4-(4- chlorobenzoyl)piperazin-1-yl)sulfonyl)-2- (hydroxycarbamoyl)-3,8- diazabicyclo[3.2.1]octane-8-carboxylate |
| 79 | 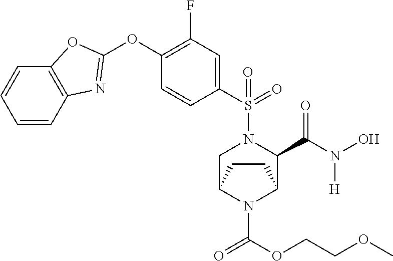 [SMILES: COCCOC(=O)N1[C@@H]2CC[C@H]1[C@H](C(=O)NO)N(S(=O)(=O)c1ccc(Oc3nc4ccccc4o3)c(F)c1)C2] | 2-methoxyethyl (1S,2R,5R)-3-((4- (benzo[d]oxazol-2-yloxy)-3- fluorophenyl)sulfonyl)-2- (hydroxycarbamoyl)-3,8- diazabicyclo[3.2.1]octane-8-carboxylate |
| 80 | 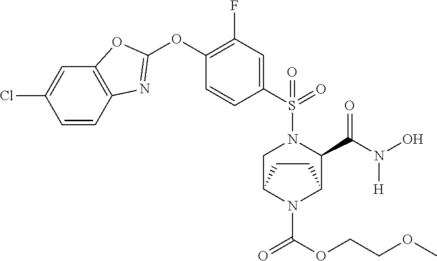 [SMILES: COCCOC(=O)N1[C@@H]2CC[C@H]1[C@H](C(=O)NO)N(S(=O)(=O)c1ccc(Oc3nc4ccc(Cl)cc4o3)c(F)c1)C2] | 2-methoxyethyl (1S,2R,5R)-3-((4-((6- chlorobenzo[d]oxazol-2-yl)oxy)-3- fluorophenyl)sulfonyl)-2- (hydroxycarbamoyl)-3,8- diazabicyclo[3.2.1]octane-8-carboxylate |
| 81 | 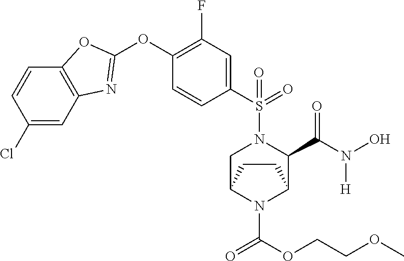 [SMILES: COCCOC(=O)N1[C@@H]2CC[C@H]1[C@H](C(=O)NO)N(S(=O)(=O)c1ccc(Oc3nc4cc(Cl)ccc4o3)c(F)c1)C2] | 2-methoxyethyl (1S,2R,5R)-3-((4-((5- chlorobenzo[d]oxazol-2-yl)oxy)-3- fluorophenyl)sulfonyl)-2- (hydroxycarbamoyl)-3,8- diazabicyclo[3.2.1]octane-8-carboxylate |
| 82 | 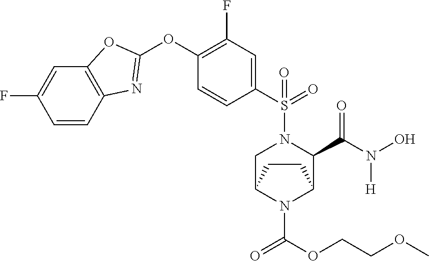 [SMILES: COCCOC(=O)N1[C@@H]2CC[C@H]1[C@H](C(=O)NO)N(S(=O)(=O)c1ccc(Oc3nc4ccc(F)cc4o3)c(F)c1)C2] | 2-methoxyethyl (1S,2R,5R)-3-((3-fluoro- 4-((6-fluorobenzo[d]oxazol-2- yl)oxy)phenyl)sulfonyl)-2- (hydroxycarbamoyl)-3,8- diazabicyclo[3.2.1]octane-8-carboxylate |
| 83 | 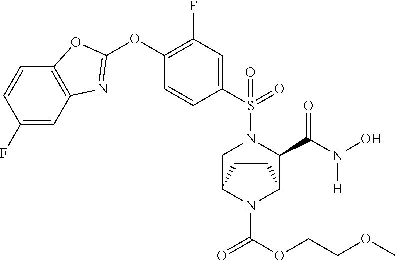 [SMILES: COCCOC(=O)N1[C@@H]2CC[C@H]1[C@H](C(=O)NO)N(S(=O)(=O)c1ccc(Oc3nc4cc(F)ccc4o3)c(F)c1)C2] | 2-methoxyethyl (1S,2R,5R)-3-((3-fluoro- 4-((5-fluorobenzo[d]oxazol-2- yl)oxy)phenyl)sulfonyl)-2- (hydroxycarbamoyl)-3,8- diazabicyclo[3.2.1]octane-8-carboxylate |
| 84 | 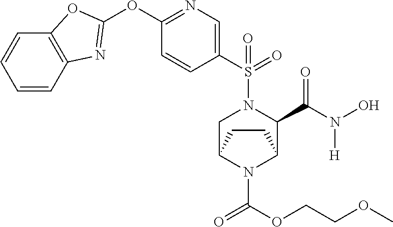 [SMILES: COCCOC(=O)N1[C@@H]2CC[C@H]1[C@H](C(=O)NO)N(S(=O)(=O)c1ccc(Oc3nc4ccccc4o3)nc1)C2] | 2-methoxyethyl (1S,2R,5R)-3-((6- (benzo[d]oxazol-2-yloxy)pyridin-3- yl)sulfonyl)-2-(hydroxycarbamoyl)-3,8- diazabicyclo[3.2.1]octane-8-carboxylate |

or is a pharmaceutically acceptable salt or solvate thereof.

In certain embodiments, a disclosed compound utilized by one or more of the foregoing methods is one of the generic, subgeneric, or specific compounds described herein, such as a compound of Formula (I) or (Ia) or (Ib) or (Ic) or (II) or (III) or (IIIa) or (IIIb) or (IIIc), described herein.

### Preparation of the Compounds

The compounds used in the methods described herein are made according to procedures disclosed herein, or by known organic synthesis techniques, starting from commercially available chemicals and/or from compounds described in the chemical literature.

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are also identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Further Forms of Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁶O, ¹⁷O, ³¹P, ³²P, ³⁵S, ¹⁸F, and ³⁶Cl, respectively. Compounds described herein, and the pharmaceutically acceptable salts, esters, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, solvate, hydrate or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

### Prodrugs

In some embodiments, the compounds described herein are formulated as agents which are converted in vivo to active forms in order to alter the biodistribution or the pharmacokinetics for a particular agent. For example, a carboxylic acid group can be esterified, e.g., with a methyl group or an ethyl group to yield an ester. When the ester is administered to a subject, the ester is cleaved, enzymatically or non enzymatically, reductively, oxidatively, or hydrolytically, to reveal the anionic group. An anionic group can be esterified with moieties (e.g., acyloxymethyl esters) which are cleaved to reveal an intermediate agent which subsequently decomposes to yield the active agent. The prodrug moieties may be metabolized in vivo by esterases or by other mechanisms to carboxylic acids. Alternatively, other functional groups may be modified into a prodrug form. For instance, an amine group may be converted into a carbamate or amide which would be cleavable in vivo.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran or methanol. In addition, the compounds provided herein exist in unsolvated as well as solvated forms. In general, the solvated forms are considered equivalent to the unsolvated forms for the purposes of the compounds and methods provided herein.

### Pharmaceutical Compositions

In certain embodiments, the compounds described herein are administered as a pure chemical. In other embodiments, the compounds described herein are combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, provided herein is a pharmaceutical composition comprising at least one compound described herein, or a pharmaceutically acceptable salt, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

One embodiment provides a pharmaceutical composition comprising a pharmaceutically acceptable carrier and a compound of Formula (I) or (Ia) or (Ib) or (Ic) or (II) or (III) or (IIIa) or (IIIb) or (IIIc), or a pharmaceutically acceptable salt thereof.

Another embodiment provides a pharmaceutical composition consisting essentially of a pharmaceutically acceptable carrier and a compound of Formula (I) or (Ta) or (Ib) or (Ic) or (II) or (III) or (IIIa) or (IIIb) or (IIIc), or a pharmaceutically acceptable salt thereof.

In certain embodiments, the compound as described herein is substantially pure, in that it contains less than about 5%, or less than about 1%, or less than about 0.1%, of other organic small molecules, such as contaminating intermediates or by-products that are created, for example, in one or more of the steps of a synthesis method.

These formulations include those suitable for oral, topical, buccal, parenteral (e.g., subcutaneous, intramuscular, intradermal, or intravenous), or aerosol administration.

Exemplary pharmaceutical compositions are used in the form of a pharmaceutical preparation, for example, in solid, semisolid or liquid form, which includes one or more of a disclosed compound, as an active ingredient, in a mixture with an organic or inorganic carrier or excipient suitable for external, enteral or parenteral applications. In some embodiments, the active ingredient is compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

In some embodiments, a compound of Formula (I) or (Ia) or (Ib) or (Ic), described herein is administered to subjects in a biologically compatible form suitable for topical administration to treat or prevent dermal diseases, disorders or conditions. By “biologically compatible form suitable for topical administration” is meant a form of the compound of Formula (I) or (Ia) or (Ib) or (Ic) or (II) or (III) or (IIIa) or (IIIb) or (IIIc), to be administered in which any toxic effects are outweighed by the therapeutic effects of the inhibitor. Administration of a compound of Formula (I) or (Ia) or (Ib) or (Ic) or (II) or (III) or (IIIa) or (IIIb) or (IIIc), as described herein can be in any pharmacological form including a therapeutically effective amount of a compound of Formula (I) or (Ia) or (Ib) or (Ic) or (II) or (III) or (IIIa) or (IIIb) or (IIIc), alone or in combination with a pharmaceutically acceptable carrier.

Topical administration of a compound of Formula (I) or (Ia) or (Ib) or (Ic) or (II) or (III) or (IIIa) or (IIIb) or (IIIc), may be presented in the form of an aerosol, a semi-solid pharmaceutical composition, a powder, or a solution. By the term “a semi-solid composition” is meant an ointment, cream, salve, jelly, or other pharmaceutical composition of substantially similar consistency suitable for application to the skin. Examples of semi-solid compositions are given in Chapter 17 of The Theory and Practice of Industrial Pharmacy, Lachman, Lieberman and Kanig, published by Lea and Febiger (1970) and in Chapter 67 of Remington's Pharmaceutical Sciences, 15th Edition (1975) published by Mack Publishing Company.

Dermal or skin patches are another method for transdermal delivery of the therapeutic or pharmaceutical compositions described herein. Patches can provide an absorption enhancer such as DMSO to increase the absorption of the compounds. Patches can include those that control the rate of drug delivery to the skin. Patches may provide a variety of dosing systems including a reservoir system or a monolithic system, respectively. The reservoir design may, for example, have four layers: the adhesive layer that directly contacts the skin, the control membrane, which controls the diffusion of drug molecules, the reservoir of drug molecules, and a water-resistant backing. Such a design delivers uniform amounts of the drug over a specified time period, the rate of delivery has to be less than the saturation limit of different types of skin.

The monolithic design, for example, typically has only three layers: the adhesive layer, a polymer matrix containing the compound, and a water-proof backing. This design brings a saturating amount of drug to the skin. Thereby, delivery is controlled by the skin. As the drug amount decreases in the patch to below the saturating level, the delivery rate falls.

In one embodiment, the topical composition may, for example, take the form of hydrogel based on polyacrylic acid or polyacrylamide; as an ointment, for example with polyethylene glycol (PEG) as the carrier, like the standard ointment DAB 8 (50% PEG 300, 50% PEG 1500); or as an emulsion, especially a microemulsion based on water-in-oil or oil-in-water, optionally with added liposomes. Suitable permeation accelerators (entraining agents) include sulfoxide derivatives such as dimethylsulfoxide (DMSO) or decylmethylsulfoxide (decyl-MSO) and transcutol (diethyleneglycolmonoethylether) or cyclodextrin; as well as pyrrolidones, for example 2-pyrrolidone, N-methyl-2-pyrrolidone, 2-pyrrolidone-5-carboxylic acid, or the biodegradable N-(2-hydroxyethyl)-2-pyrrolidone and the fatty acid esters thereof; urea derivatives such as dodecylurea, 1,3-didodecylurea, and 1,3-diphenylurea; terpenes, for example D-limonene, menthone, α-terpineol, carvol, limonene oxide, or 1,8-cineol.

Ointments, pastes, creams and gels also can contain excipients, such as starch, tragacanth, cellulose derivatives, polyethylene glycols, silicones, bentonites, silicic acid, and talc, or mixtures thereof. Powders and sprays also can contain excipients such as lactose, talc, silicic acid, aluminum hydroxide, calcium silicates and polyamide powder, or mixtures of these substances. Solutions of nanocrystalline antimicrobial metals can be converted into aerosols or sprays by any of the known means routinely used for making aerosol pharmaceuticals. In general, such methods comprise pressurizing or providing a means for pressurizing a container of the solution, usually with an inert carrier gas, and passing the pressurized gas through a small orifice. Sprays can additionally contain customary propellants, such a chlorofluorohydrocarbons and volatile unsubstituted hydrocarbons, such as butane and propane.

In some embodiments for preparing solid compositions such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a disclosed compound or a non-toxic pharmaceutically acceptable salt thereof. When referring to these preformulation compositions as homogeneous, it is meant that the active ingredient is dispersed evenly throughout the composition so that the composition is readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, cellulose, microcrystalline cellulose, silicified microcrystalline cellulose, lactose, sucrose, glucose, mannitol, and/or silicic acid; (2) binders, such as, for example, carboxymethylcellulose, hypromellose, alginates, gelatin, polyvinyl pyrrolidone, sucrose and/or acacia; (3) humectants, such as glycerol; (4) disintegrating agents, such as crospovidone, croscarmellose sodium, sodium starch glycolate, agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, docusate sodium, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, in some embodiments, the compositions comprise buffering agents. In some embodiments, solid compositions of a similar type are also employed as fillers in soft and hard-filled gelatin capsules using such excipients as lactose or milk sugars, as well as high molecular weight polyethylene glycols and the like.

In some embodiments, a tablet is made by compression or molding, optionally with one or more accessory ingredients. In some embodiments, compressed tablets are prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. In some embodiments, molded tablets are made by molding in a suitable machine a mixture of the subject composition moistened with an inert liquid diluent. In some embodiments, tablets, and other solid dosage forms, such as dragées, capsules, pills and granules, are scored or prepared with coatings and shells, such as enteric coatings and other coatings.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, in some embodiments, the liquid dosage forms contain inert diluents, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

In some embodiments, suspensions, in addition to the subject composition, contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some embodiments, powders and sprays contain, in addition to a subject composition, excipients such as lactose, talc, silicic acid, aluminum hydroxide, calcium silicates and polyamide powder, or mixtures of these substances. In some embodiments, sprays additionally contain customary propellants, such as chlorofluorohydrocarbons and volatile unsubstituted hydrocarbons, such as butane and propane.

Compositions and compounds disclosed herein alternatively are administered by aerosol. This is accomplished by preparing an aqueous aerosol, liposomal preparation or solid particles containing the compound. In some embodiments, a non-aqueous (e.g., fluorocarbon propellant) suspension is used. In some embodiments, sonic nebulizers are used because they minimize exposing the agent to shear, which results in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which are reconstituted into sterile injectable solutions or dispersions just prior to use, which, in some embodiments, contain antioxidants, buffers, bacteriostats, solutes which render the formulation isotonic with the blood of the intended recipient or suspending or thickening agents.

Examples of suitable aqueous and non-aqueous carriers which are employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity is maintained, for example, by the use of coating materials, such as lecithin, by the maintenance of the required particle size in the case of dispersions, and by the use of surfactants

The dose of the composition comprising at least one compound described herein differs, depending upon the patient's (e.g., human) condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). Optimal doses are generally determined using experimental models and/or clinical trials. In some embodiments, the optimal dose depends upon the body mass, weight, or blood volume of the patient.

Oral doses typically range from about 1.0 mg to about 1000 mg, one to four times, or more, per day.

### Methods

In some embodiments disclosed herein is a method for treating a disease, disorder or condition selected from:

a cardiovascular disease, heart failure, congestive heart failure, heart failure with reduced ejection fraction, heart failure with reserved ejection fraction, impaired cardiac contractility, age-related cardiac hypertrophy, inflammation and fibrosis, viral myocarditis, COVID-19 myocarditis, COVID-19 related myocardial fibrosis, pressure overload hypertrophy, myocardial fibrosis, myocardial infarction, myocardial ischemia/reperfusion injury, pathological remodeling of myocardium, ECM remodeling following myocardial injury, radiation myocarditis, radiation myocardial fibrosis, chemotherapy cardiomyopathy, vessel rarefaction, aortic valve sclerosis, calcific aortic valve stenosis, aortic aneurism, abdominal aorta aneurism, giant cell arteritis, age-associated arterial fibrosis, pulmonary hypertension, right ventricle hypertrophy,
idiopathic pulmonary fibrosis, acute lung injury (ALI), acute respiratory distress syndrome (ARDS), Hermansky-Pudlak syndrome (HPS), chronic obstructive pulmonary disease (COPD), emphysema,
polycystic kidney disease, membranous nephropathy, diabetic nephropathy, acute kidney injury, glomerulonephritis, inherited kidney disease, and chronic allograft nephropathy, focal segmental glomerulosclerosis, minimal change disease, human immunodeficiency virus-associated nephropathy, anti-neutrophil cytoplasmic antibody-associated vasculitis, lupus nephritis, IgA nephropathy, Henoch-Schoenlein purpura, and postinfectious glomerulonephritis, membranoproliferative glomerulonephritis, cisplatin-induced renal injury, tubular injury following sepsis], acute ischemic kidney injury, contrast-induced kidney injury, acute tubular injury after ischemia and reperfusion, end-stage renal disease, tubulointerstitial fibrosis, alcoholic liver disease, non-alcoholic fatty liver disease, non-alcoholic steatohepatitis, steatosis, cirrhosis, hepatic ischemia and reperfusion injury, viral hepatitis, drug-induced liver injury, primary biliary cholangitis, primary sclerosing cholangitis, hemochromatosis, Wilson's disease, acute liver failure, biliary atresia,
and scleroderma pigmentosum,
in a mammal in need thereof, comprising administering to the mammal in need thereof a therapeutically effective amount of a compound of Formula (I) or (Ia) or (Ib) or (Ic) or (II) or (III) or (IIIa) or (IIIb) or (IIIc), or a pharmaceutically acceptable salt or solvate thereof.

In some embodiments disclosed herein is a method for treating a cardiovascular disease, disorder or condition in a mammal in need thereof, comprising administering to the mammal in need thereof a therapeutically effective amount of a compound of Formula (Ta), or a pharmaceutically acceptable salt or solvate thereof.

In some embodiments disclosed herein is a method for treating a cardiovascular disease, disorder or condition in a mammal in need thereof, comprising administering to the mammal in need thereof a therapeutically effective amount of a compound of Formula (Ib), or a pharmaceutically acceptable salt or solvate thereof.

In some embodiments disclosed herein is a method for treating a cardiovascular disease, disorder or condition in a mammal in need thereof, comprising administering to the mammal in need thereof a therapeutically effective amount of a compound of Formula (Ic), or a pharmaceutically acceptable salt or solvate thereof.

In some embodiments disclosed herein is a method for treating a cardiovascular disease, disorder or condition in a mammal in need thereof, comprising administering to the mammal in need thereof a therapeutically effective amount of a compound of Formula (II), or a pharmaceutically acceptable salt or solvate thereof.

In some embodiments disclosed herein is a method for treating a cardiovascular disease, disorder or condition in a mammal in need thereof, comprising administering to the mammal in need thereof a therapeutically effective amount of a compound of Formula (III), or a pharmaceutically acceptable salt or solvate thereof.

In some embodiments disclosed herein is a method for treating a cardiovascular disease, disorder or condition in a mammal in need thereof, comprising administering to the mammal in need thereof a therapeutically effective amount of a compound of Formula (IIIa), or a pharmaceutically acceptable salt or solvate thereof.

In some embodiments disclosed herein is a method for treating a cardiovascular disease, disorder or condition in a mammal in need thereof, comprising administering to the mammal in need thereof a therapeutically effective amount of a compound of Formula (IIIb), or a pharmaceutically acceptable salt or solvate thereof.

In some embodiments disclosed herein is a method for treating a cardiovascular disease, disorder or condition in a mammal in need thereof, comprising administering to the mammal in need thereof a therapeutically effective amount of a compound of Formula (IIIc), or a pharmaceutically acceptable salt or solvate thereof.

### Pharmaceutical Combinations

Also contemplated herein are combination therapies, for example, co-administering a disclosed compound and an additional active agent, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner.

Substantially simultaneous administration is accomplished, for example, by administering to the subject a single formulation or composition, (e.g., a tablet or capsule having a fixed ratio of each therapeutic agent or in multiple, single formulations (e.g., capsules) for each of the therapeutic agents. Substantially simultaneous administration is also accomplished by administering to the subject a new chemical entity that is comprised of disclosed compound bound through chemical bond or linker to one or more additional active agents. Sequential or substantially simultaneous administration of each therapeutic agent is effected by any appropriate route including, but not limited to, oral routes, intravenous routes, intramuscular routes, and direct absorption through mucous membrane tissues. The therapeutic agents are administered by the same route or by different routes. For example, a first therapeutic agent of the combination selected is administered by intravenous injection while the other therapeutic agents of the combination are administered orally. Alternatively, for example, all therapeutic agents are administered orally or all therapeutic agents are administered by intravenous injection.

In some embodiments, disclosed herein is a method of treating a disease, disorder or condition selected from:
a cardiovascular disease, heart failure, congestive heart failure, heart failure with reduced ejection fraction, heart failure with reserved ejection fraction, impaired cardiac contractility, age-related cardiac hypertrophy, inflammation and fibrosis, viral myocarditis, COVID-19 myocarditis, COVID-19 related myocardial fibrosis, pressure overload hypertrophy, myocardial fibrosis, myocardial infarction, myocardial ischemia/reperfusion injury, pathological remodeling of myocardium, ECM remodeling following myocardial injury, radiation myocarditis, radiation myocardial fibrosis, chemotherapy cardiomyopathy, vessel rarefaction, aortic valve sclerosis, calcific aortic valve stenosis, aortic aneurism, abdominal aorta aneurism, giant cell arteritis, age-associated arterial fibrosis, pulmonary hypertension, right ventricle hypertrophy,
idiopathic pulmonary fibrosis, acute lung injury (ALI), acute respiratory distress syndrome (ARDS), Hermansky-Pudlak syndrome (HPS), chronic obstructive pulmonary disease (COPD), emphysema,
polycystic kidney disease, membranous nephropathy, diabetic nephropathy, acute kidney injury, glomerulonephritis, inherited kidney disease, and chronic allograft nephropathy, focal segmental glomerulosclerosis, minimal change disease, human immunodeficiency virus-associated nephropathy, anti-neutrophil cytoplasmic antibody-associated vasculitis, lupus nephritis, IgA nephropathy, Henoch-Schoenlein purpura, and postinfectious glomerulonephritis, membranoproliferative glomerulonephritis, cisplatin-induced renal injury, tubular injury following sepsis], acute ischemic kidney injury, contrast-induced kidney injury, acute tubular injury after ischemia and reperfusion, end-stage renal disease, tubulointerstitial fibrosis, alcoholic liver disease, non-alcoholic fatty liver disease, non-alcoholic steatohepatitis, steatosis, cirrhosis, hepatic ischemia and reperfusion injury, viral hepatitis, drug-induced liver injury, primary biliary cholangitis, primary sclerosing cholangitis, hemochromatosis, Wilson's disease, acute liver failure, biliary atresia,
and scleroderma pigmentosum,
in a mammal in need thereof, comprising administering to the mammal in need thereof a therapeutically effective amount of a compound of Formula (I) or (Ia) or (Ib) or (Ic) or (II) or (III) or (IIIa) or (IIIb) or (IIIc), or a pharmaceutically acceptable salt or solvate thereof, further comprising administering to the mammal one or more additional pharmaceutical agents.
   In some embodiments, disclosed is a method of treating a cardiovascular disease, disorder or condition in a mammal in need thereof, comprising administering to the mammal in need thereof a therapeutically effective amount of a compound of Formula (I), further comprising administering to the mammal one or more additional pharmaceutical agents. In some embodiments, disclosed is a method of treating a cardiovascular disease, disorder or condition in a mammal in need thereof, comprising administering to the mammal in need thereof a therapeutically effective amount of a compound of Formula (Ia), further comprising administering to the mammal one or more additional pharmaceutical agents. In some embodiments, disclosed is a method of treating a cardiovascular disease, disorder or condition in a mammal in need thereof, comprising administering to the mammal in need thereof a therapeutically effective amount of a compound of Formula (Ib), further comprising administering to the mammal one or more additional pharmaceutical agents. In some embodiments, disclosed is a method of treating a cardiovascular disease, disorder or condition in a mammal in need thereof, comprising administering to the mammal in need thereof a therapeutically effective amount of a compound of Formula (Ic), further comprising administering to the mammal one or more additional pharmaceutical agents. In some embodiments, disclosed is a method of treating a cardiovascular disease, disorder or condition in a mammal in need thereof, comprising administering to the mammal in need thereof a therapeutically effective amount of a compound of Formula (II), further comprising administering to the mammal one or more additional pharmaceutical agents. In some embodiments, disclosed is a method of treating a cardiovascular disease, disorder or condition in a mammal in need thereof, comprising administering to the mammal in need thereof a therapeutically effective amount of a compound of Formula (III), further comprising administering to the mammal one or more additional pharmaceutical agents. In some embodiments, disclosed is a method of treating a cardiovascular disease, disorder or condition in a mammal in need thereof, comprising administering to the mammal in need thereof a therapeutically effective amount of a compound of Formula (IIIa), further comprising administering to the mammal one or more additional pharmaceutical agents. In some embodiments, disclosed is a method of treating a cardiovascular disease, disorder or condition in a mammal in need thereof, comprising administering to the mammal in need thereof a therapeutically effective amount of a compound of Formula (IIIb), further comprising administering to the mammal one or more additional pharmaceutical agents. In some embodiments, disclosed is a method of treating a cardiovascular disease, disorder or condition in a mammal in need thereof, comprising administering to the mammal in need thereof a therapeutically effective amount of a compound of Formula (IIIc), further comprising administering to the mammal one or more additional pharmaceutical agents.

Combination therapy also embraces the administration of the therapeutic agents as described above in further combination with other biologically active ingredients and non-drug therapies. Where the combination therapy further comprises a non-drug treatment, the non-drug treatment is conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The components of the combination are administered to a patient simultaneously or sequentially. It will be appreciated that the components are present in the same pharmaceutically acceptable carrier and, therefore, are administered simultaneously.

Alternatively, the active ingredients are present in separate pharmaceutical carriers, such as conventional oral dosage forms, that are administered either simultaneously or sequentially.

### EXAMPLES

The present invention is further illustrated by the following examples, which in no way should be construed as limiting the scope of the claims provided herein.

### List of Abbreviations

As used above, and throughout the description of the invention, the following abbreviations, unless otherwise indicated, shall be understood to have the following meanings:
s singlet
d doublet
t triplet
q quartet
m multiplet
brs broad singlet
dd doublet of doublets
td triplet of doublets
dt doublet of triplets
° C. degrees centigrade
ACN acetonitrile
Bn benzyl
BOC or Boc tert-butoxycarbonyl
(Boc)₂O di-tert-butyl dicarbonate
BOP—Cl bis(2-oxo-3-oxazolidinyl)phosphinic chloride
CDI 1,1′-carbonyldiimidazole
Cy cyclohexyl
DCE dichloroethane (ClCH₂CH₂Cl)
DCM dichloromethane, methylene chloride (CH₂Cl₂)
DEAD diethyl azodicarboxylate
DIPEA N,N-diisopropylethylamine
DMA N,N-dimethylacetamide
DMAP 4-(N,N-dimethylamino)pyridine
DMF N,N-dimethylformamide
DMSO dimethylsulfoxide
equiv equivalent(s)
Et ethyl
EtOH ethanol
EA or EtOAc ethyl acetate
g gram(s)
h hour(s)
HATU 1-[bis(dimethylamino)methylene]-1H-1,2,3-triazolo[4,5-b]pyridinium 3-oxide hexafluorophosphate
HPLC high performance liquid chromatography
i-PrOAc isopropyl acetate
J NMR coupling constant in Hertz (Hz)
LAH lithium aluminum hydride
LCMS liquid chromatography followed by mass spectroscopy
Me methyl
MeOH methanol
mL milliliter(s)
μL microliter(s)
mmol millimole(s)
μmol micromole(s)
MS mass spectroscopy
MTBE methyl tert-butyl ether
NaH sodium hydride (60% dispersion in mineral oil)
NMM N-methylmorpholine
NMR nuclear magnetic resonance
PDA photodiode array detector
Pd/C 10% palladium on activated charcoal, 50% wet
PMB p-methoxybenzyl
p-TSA p-toluenesulfonic acid monohydrate
RT or rt room temperature
TEA or Et₃N triethylamine
TBAF tetrabutylammonium fluoride
TFA trifluoroacetic acid
THF tetrahydrofuran
TLC thin layer chromatography

If not otherwise defined, purity of a solid substance is expressed as a ratio of the weight of the component in question to the total weight, multiplied by 100 (weight %); purity of a liquid is expressed as a ratio of the volume of the component in question to the total volume, multiplied by 100 (volume %); concentration of a solution is expressed as a ratio of the weight of the solute (in grams) to the total volume (in mL) of the solution, multiplied by 100(% w/v). Yield of a reaction is expressed as a ratio of the weight of the product in question to the theoretical yield of this product, multiplied by 100(%). Composition of a mixed solvent is expressed as a proportion of volume parts of the component solvents (e.g., 80:20 or 3:2:1).

### GENERAL REMARKS

Commercially available chemicals were obtained from standard commercial sources including Acros Organics (Geel, Belgium), Aldrich Chemical (Milwaukee, WI, including Sigma Chemical and Fluka), Apin Chemicals Ltd. (Milton Park, UK), Ark Pharm, Inc. (Libertyville, IL), Avocado Research (Lancashire, U.K.), BDH Inc. (Toronto, Canada), Bionet (Cornwall, U.K.), Chemservice Inc. (West Chester, PA), Combi-blocks (San Diego, CA), Crescent Chemical Co. (Hauppauge, NY), eMolecules (San Diego, CA), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Matrix Scientific, (Columbia, SC), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen A G (Hanover, Germany), Ryan Scientific, Inc. (Mount Pleasant, SC), Spectrum Chemicals (Gardena, CA), Sundia Meditech, (Shanghai, China), TCI America (Portland, OR), Trans World Chemicals, Inc. (Rockville, MD), and WuXi (Shanghai, China). Dry solvents like DCM, diethyl ether and toluene were procured from Finar and used as such whereas THF was dried over sodium (monitored by color of benzophenone ketyl radical) and freshly distilled prior to use. Liquids and solutions were transferred via syringe or cannula. All reactions were carried out in flame-dried or oven dried glassware under an atmosphere of nitrogen/argon with magnetic stirring. All reactions were monitored by thin-layer chromatography (TLC) with Merck silica gel 60 F₂₅₄ pre-coated plates (0.25 mm) and visualized using UV light, iodine, potassium permanganate stain, p-anisaldehyde stain or phosphomolybdic acid stain; GC/FID; LCMS; or ¹H NMR. Crude compounds were purified by typical laboratory methods including: trituration; silica gel (60-120, 100-200 or 230-400 mesh) chromatography; and combi flash normal phase or reverse phase.

### Analytical Methods

Proton (¹H) and carbon (¹³C) NMR spectra were recorded on a Bruker Ascend-400 spectrometer operating at 400 MHz for proton and 100 MHz for carbon using CDCl₃, DMSO-d₆, CD₃CN or D₂O as solvents. Chemical shifts are expressed as parts per million (6, ppm) and coupling constants (J) are reported in Hertz. For proton spectra, the solvent peak was used as the reference peak. LCMS was performed on Shimadzu LCMS-2020 or Agilent 6420 Triple Quad LC/MS or Agilent Infinity Lab LC/MSD XT using PDA detector under the conditions of electrospray ionization (ESI) in both positive and negative mode. HPLC was performed on Shimadzu LC-2010 and Agilent 1290 Infinity II using PDA detector. Preparative HPLC was performed on Shimadzu semi preparative or Agilent 1260 Infinity II using PDA detector. Column chromatography and thin layer chromatography (TLC) were performed on silica gel unless otherwise noted.

The operating conditions of chromatographic and mass-spectral analyses are summarized in Tables 1-3 below.

TABLE 1
LCMS conditions

| Method | Conditions |
|---|---|
| 1 | Column: Kinetex EVO, C-18 (2.1 × 50 mm × 1.7 μm); Mobile phase-A: 0.01% |
| | Formic acid in deionized water; Mobile phase-B: ACN; Detector: PDA; Flow |
| | rate: 0.6 mL/min; Sample concentration: 0.3 mg/mL; Gradient (T/% B): 0/5, |
| | 7/95, 10/95, 10.1/05, 11.5/05; Run time: 11.5 minutes; Diluent: Water:ACN |
| | (80:20). |
| 2 | Column: Kinetex EVO, C-18 (2.1 × 50 mm × 1.7 μm); Mobile phase-A: 0.01% |
| | Formic acid in deionized water; Mobile phase-B: ACN; Detector: PDA; Flow |
| | rate: 0.6 mL/min; Sample concentration: 0.3 mg/mL; Gradient (T/% B): 0/5, |
| | 1.5/95, 5/95, 5.1/5, 6.5/5; Run time: 6.5 minutes; Diluent: Water:ACN (80:20). |
| 3 | Column: Luna, C-18 (2.1 × 50 mm × 3.0 μm); Mobile phase-A: 0.01% Formic |
| | acid in deionized water; Mobile phase-B: ACN; Detector: PDA; Flow rate: 0.6 |
| | mL/min; Sample concentration: 0.3 mg/mL; Gradient (T/% B): 0/5, 6/95, 10/95, |
| | 10.1/5, 12/5; Run time: 12 minutes; Diluent: Water:ACN (80:20). |
| 4 | Column: X-Bridge, C-18 (4.6 × 150 mm × 3.0 μm); Mobile phase-A: 5.0 mM |
| | ammonium bicarbonate in deionized water; Mobile phase-B: ACN; Detector: |
| | PDA; Flow rate: 0.8 mL/min; Sample concentration: 0.3 mg/mL; Gradient |
| | (T/% B): 0/10, 6/95, 12/95, 12.1/10, 15/10; Run time: 15.0 minutes; Diluent: |
| | Water:ACN (80:20). |

TABLE 2
Preparative HPLC conditions

| Method | Conditions |
|---|---|
| 1 | Column: Gemini C-18 (250 × 21.2 mm × 5 μm); Mobile phase-A: 0.01% Formic |
| | acid in deionized water; Mobile phase-B: ACN; Detector: PDA; Gradient |
| | (T/% B): 0/35, 10/40, 20/70, 25/55; Diluent: ACN/Water; Run time: 25 minutes; |
| | Flow rate: 16.0 mL/min. |
| 2 | Column: Luna C-18 (250 × 21.2 mm × 5 μm); Mobile phase-A: 0.01% Formic |
| | acid buffer in deionized water; Mobile phase-B: ACN; Detector: PDA; Gradient |
| | (T/% B): 0/60, 10/70, 25/80; Diluent: ACN/Water; Run time: 25 minutes; Flow |
| | rate: 16.0 mL/min. |
| 3 | Column: Kinetex C-18 (250 × 21.2 mm × 5 μm); Mobile phase-A: 0.01% Formic |
| | acid buffer in deionized water; Mobile phase-B: ACN; Detector: PDA; Gradient |
| | (T/% B): 0/35, 12/45, 15/60; Diluent: ACN/Water; Run time: 15 minutes; Flow |
| | rate: 16.0 mL/min. |
| 4 | Column: Kinetex C-18 (250 × 21.2 mm × 5 μm); Mobile phase-A: 0.01% |
| | Ammonium acetate buffer in deionized water; Mobile phase-B: ACN; |
| | Wavelength: PDA; Gradient (T/% B): 0/30, 15/40, 25/55; Diluent: ACN/Water; |
| | Run time: 25 minutes; Flow rate: 16.0 mL/min. |
| 5 | Column: Gemini C-18 (250 × 21.2 mm × 5 μm); Mobile phase-A: 0.01% |
| | Ammonium acetate buffer in deionized water; Mobile phase-B: ACN; |
| | Wavelength: PDA; Gradient (T/% B): 0/30, 15/50; Diluent: ACN/Water; Run |
| | time: 15 minutes; Flow rate: 16.0 mL/min. |
| 6 | Column: Gemini C-18 (250 × 21.2 mm × 5 μm); Mobile phase-A: 0.01% |
| | Ammonium bicarbonate buffer in deionized water; Mobile phase-B: ACN; |
| | Wavelength: PDA; Gradient (T/% B): 0.1/30, 5/40, 15/50, 35/95; Diluent: |
| | ACN/Water; Run time: 35 minutes; Flow rate: 16.0 mL/min. |

TABLE 3
HPLC conditions

| Method | Conditions |
|---|---|
| 1 | Column: Gemini, C-18 (250 × 4.6 mm × 5.0 μ); Mobile phase-A: 5.0 mM |
| | ammonium bicarbonate in deionized water; Mobile phase-B: Acetonitrile; |
| | Detector: PDA; Flow rate: 1.0 mL/min; Sample concentration: 0.5 mg/mL; |
| | Gradient (T/% B): 0/05, 13/90, 20/90, 21/05, 25/05; Run time: 25 mins; Diluent: |
| | Water:ACN (50:50). |
| 2 | Column: Kinetex EVO, C-18 (250 × 4.6 mm × 5.0 μ); Mobile phase-A: 0.01% |
| | TFA in deionized water; Mobile phase-B: Acetonitrile; Detector: PDA; Flow |
| | rate: 1.0 mL/min; Sample concentration: 0.5 mg/mL; Gradient (T/% B): 0/05, |
| | 20/90, 30/90, 31/05, 35/05; Run time: 35 mins; Diluent: Water:ACN (50:50). |
| 3 | Column: Gemini, C-18 (250 × 4.6 mm × 5.0 μ); Mobile phase-A: 5.0 mM |
| | ammonium acetate in deionized water; Mobile phase-B: Acetonitrile; Detector: |
| | PDA; Flow rate: 1.0 mL/min; Sample concentration: 0.5 mg/mL; Gradient |
| | (T/% B): 0/05, 13/90, 20/90, 21/05, 25/05; Run time: 25 mins; Diluent: |
| | Water:ACN (50:50). |

### Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized.

Compounds according to the present invention may be prepared using the synthetic transformations illustrated in Synthetic Schemes 1 to 10 below. Starting materials are commercially available, may be prepared by the procedures described herein, by literature procedures, or by procedures that would be well known to one skilled in the art of organic chemistry.

Exemplary synthetic methodologies for making compounds according to this disclosure are provided below.

In Step 1 of the General Synthetic Scheme 1, various sulfonyl chlorides (1B) react with the secondary amine (1A) to afford Formula 1C. Formula 1C undergoes deprotection to afford Formula 1D. Further Formula 1D is reacted with methoxyethyl chloroformate to afford Formula 1E. Further Formula 1E was treated with inorganic base in water and THF to afford Formula 1F, which on further coupling with protected hydroxylamine under usual amide conditions yielded Formula 1G. Finally, Formula 1G undergoes deprotection in presence of suitable acids such as TFA, 1N HCl etc. in suitable solvents such as DCM, 1,4-dioxane, and MeOH to provide Formula 1.

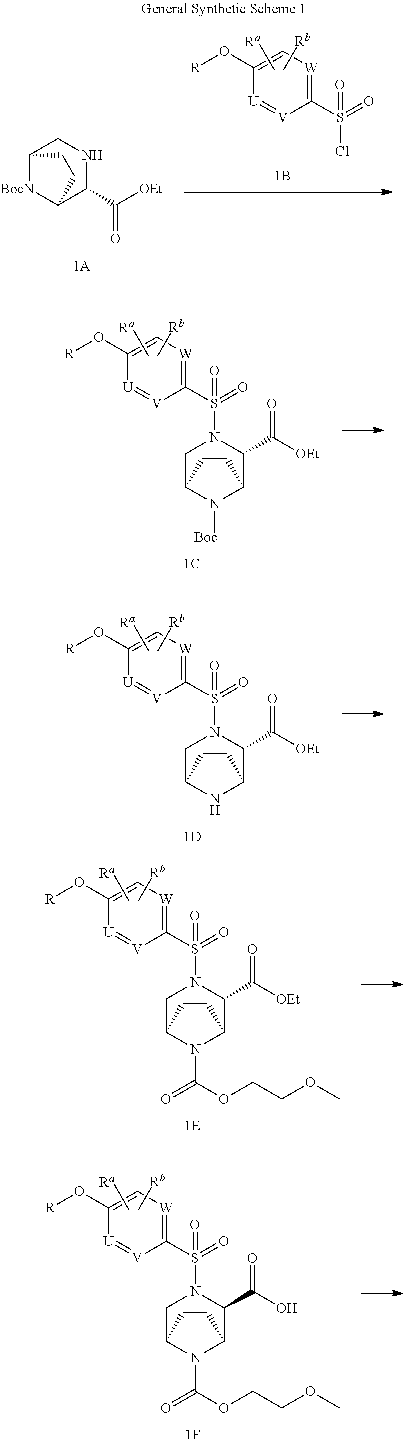

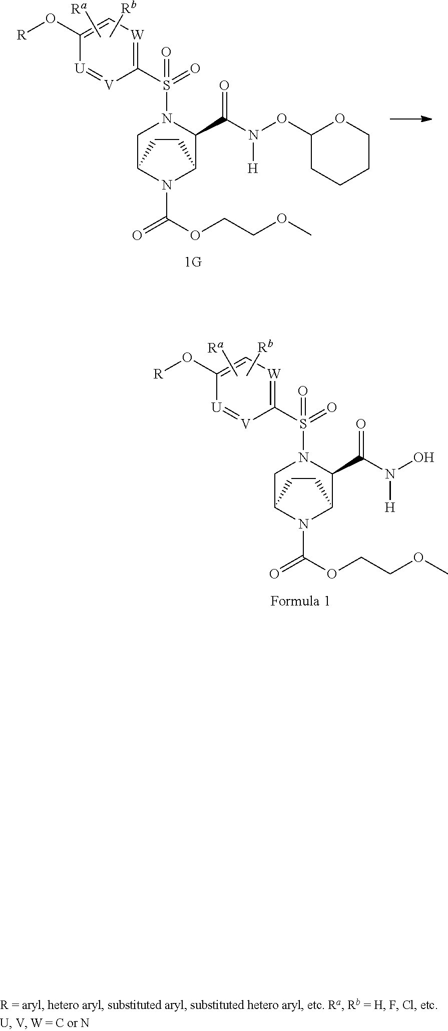

### Example 1

### Synthesis of (1S,2R,5R)-2-methoxyethyl 3-((6-(4-fluorophenoxy)pyridin-3-yl)sulfonyl)-2-(hydroxycarbamoyl)-3,8-diazabicyclo[3.2.1]octane-8-carboxylate

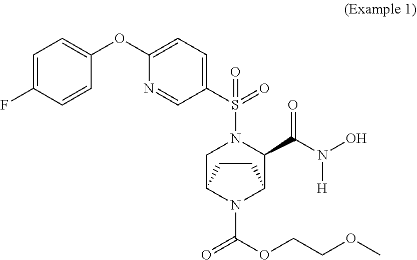 [SMILES: COCCOC(=O)N1[C@@H]2CC[C@H]1[C@H](C(=O)NO)N(S(=O)(=O)c1ccc(Oc3ccc(F)cc3)nc1)C2]

### Step 1

### Synthesis of (1S,2S,5R)-8-tert-butyl 2-ethyl 3-((6-(4-fluorophenoxy)pyridin-3-yl)sulfonyl)-3,8-diazabicyclo[3.2.1]octane-2,8-dicarboxylate

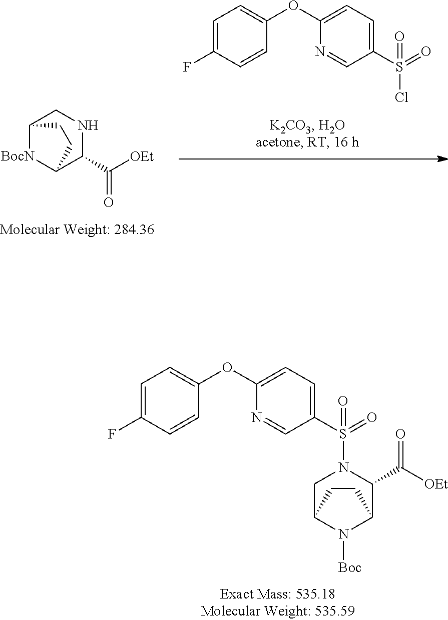 [SMILES: O=S(=O)(Cl)c1ccc(Oc2ccc(F)cc2)nc1 ; CCOC(=O)[C@H]1NC[C@H]2CC[C@@H]1N2C(=O)OC(C)(C)C ; CCOC(=O)[C@@H]1[C@@H]2CC[C@H](CN1S(=O)(=O)c1ccc(Oc3ccc(F)cc3)nc1)N2C(=O)OC(C)(C)C]

To a stirred solution of (1S,2S,5R)-8-tert-butyl 2-ethyl 3,8-diazabicyclo[3.2.1]octane-2,8-dicarboxylate (300 mg, 1.05 mmol) in acetone (6 mL) under nitrogen atmosphere at 0-10° C., was added K₂CO₃ (580 mg, 4.2 mmol) and stirred for 20 min. 6-(4-Fluorophenoxy)pyridine-3-sulfonyl chloride (450 mg, 1.58 mmol) was dissolved in acetone (4 mL) and added to the reaction mixture at the same temperature. Then, water (0.05 mL) was added and the reaction mixture was stirred at room temperature for 16 h while being monitored by TLC. The reaction mixture was diluted with water (10 mL) and extracted with ethyl acetate (2×50 mL). The organic layers were combined, dried over anhydrous Na₂SO₄, filtered and concentrated under reduced pressure to obtain crude product. The crude product was purified by silica gel column chromatography (100-200 mesh, 15-20% EtOAc in hexane) to afford (1S,2S,5R)-8-tert-butyl 2-ethyl 3-((6-(4-fluorophenoxy)pyridin-3-yl)sulfonyl)-3, 8-diazabicyclo[3.2.1]octane-2,8-dicarboxylate (330 mg, 0.61 mmol, 58% yield) as a colorless sticky gum. MS (ESI): m/z 480.0 [M-t-Bu+H]⁺.

### Step 2

### (1S,2S,5R)-ethyl 3-((6-(4-fluorophenoxy)pyridin-3-yl)sulfonyl)-3,8-diazabicyclo[3.2.1]octane-2-carboxylate

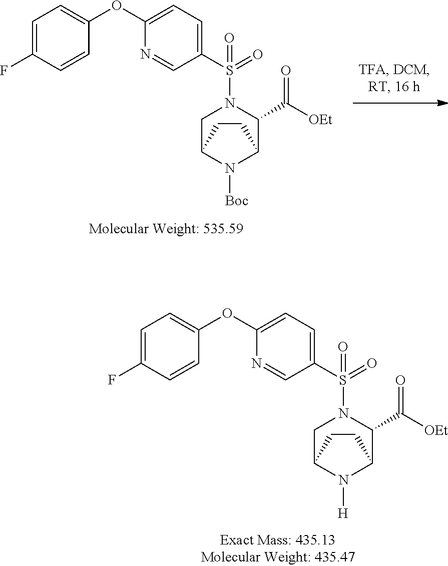 [SMILES: CCOC(=O)[C@@H]1[C@@H]2CC[C@H](CN1S(=O)(=O)c1ccc(Oc3ccc(F)cc3)nc1)N2C(=O)OC(C)(C)C ; CCOC(=O)[C@@H]1[C@@H]2CC[C@H](CN1S(=O)(=O)c1ccc(Oc3ccc(F)cc3)nc1)N2]

To a stirred solution of (1S,2S,5R)-8-tert-butyl 2-ethyl 3-((6-(4-fluorophenoxy)pyridin-3-yl)sulfonyl)-3,8-diazabicyclo[3.2.1]octane-2,8-dicarboxylate (0.33 g, 0.61 mmol) in DCM (4 mL) at 0-5° C., was added trifluoroacetic acid (0.5 mL, 6.1 mmol) dropwise and stirred at room temperature for 16 h while being monitored by TLC. The reaction mixture was evaporated to dryness, the residue was diluted with ethyl acetate (40 mL) and washed with saturated NaHCO₃ solution (2×15 mL). The organic layer was separated, dried over anhydrous Na₂SO₄, filtered and concentrated under reduced pressure to obtain (1S,2S,5R)-ethyl 3-((6-(4-fluorophenoxy)pyridin-3-yl)sulfonyl)-3,8-diazabicyclo[3.2.1]octane-2-carboxylate (270 mg, 0.62 mmol, quantitative) as a colorless sticky gum. This was used as such for the next step without further purification. MS (ESI): m/z 436.0 [M+H]⁺.

### Step 3

### Synthesis of (1S,2S,5R)-2-ethyl 8-(2-methoxyethyl) 3-((6-(4-fluorophenoxy)pyridin-3-yl)sulfonyl)-3,8-diazabicyclo[3.2.1]octane-2,8-dicarboxylate

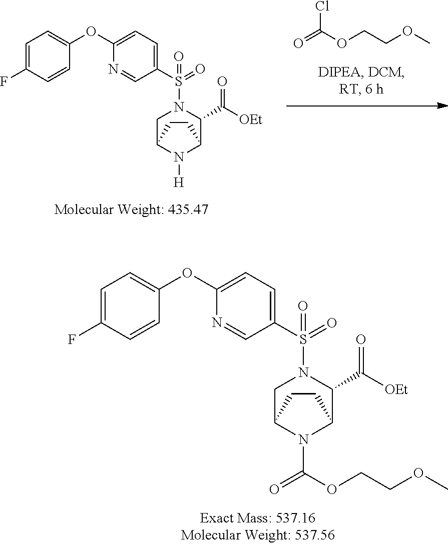

To a stirred solution of (1S,2S,5R)-ethyl 3-((6-(4-fluorophenoxy)pyridin-3-yl)sulfonyl)-3,8-diazabicyclo[3.2.1]octane-2-carboxylate (270 mg, 0.62 mmol) in DCM (4 mL) at 0-10° C., was added DIPEA (0.43 mL, 2.48 mmol) and 2-methoxyethyl chloroformate (0.14 mL, 1.24 mmol) under N₂ atmosphere. The reaction mixture was then stirred at RT for 6 h while being monitored by TLC. The reaction mixture was quenched with ice cold water (5 mL) and extracted into DCM (2×20 mL). The combined organic layer was washed with ice cold water (2×5 mL), dried over anhydrous Na₂SO₄, filtered and concentrated under reduced pressure to obtain (1S,2S,5R)-2-ethyl 8-(2-methoxyethyl) 3-((6-(4-fluorophenoxy)pyridin-3-yl)sulfonyl)-3,8-diazabicyclo[3.2.1]octane-2,8-dicarboxylate (340 mg, 0.63 mmol, quantitative) as a colorless sticky gum. This was used as such for the next step without further purification. MS (ESI): m/z 538.0 [M+H]⁺.

### Step 4

### Synthesis of (1S,2R,5R)-3-((6-(4-fluorophenoxy)pyridin-3-yl)sulfonyl)-8-((2-methoxyethoxy)carbonyl)-3,8-diazabicyclo[3.2.1]octane-2-carboxylic acid

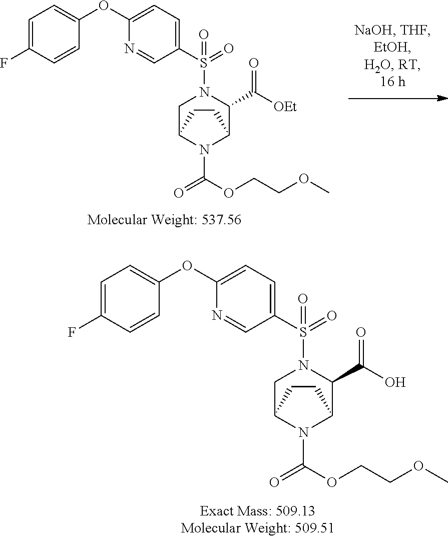 [SMILES: CCOC(=O)[C@@H]1[C@@H]2CC[C@H](CN1S(=O)(=O)c1ccc(Oc3ccc(F)cc3)nc1)N2C(=O)OCCOC ; COCCOC(=O)N1[C@@H]2CC[C@H]1[C@H](C(=O)O)N(S(=O)(=O)c1ccc(Oc3ccc(F)cc3)nc1)C2]

To a stirred solution of (1S,2S,5R)-2-ethyl 8-(2-methoxyethyl) 3-((6-(4-fluorophenoxy)pyridin-3-yl)sulfonyl)-3,8-diazabicyclo[3.2.1]octane-2,8-dicarboxylate (330 mg, 0.61 mmol) in 1:1:1 mixture of THF:EtOH:H₂O (30 mL) at 0° C., was added NaOH (122 mg, 3.0 mmol) in one portion. The reaction mixture was stirred at RT for 16 h while being monitored by TLC. The volatiles were evaporated under reduced pressure, the residue was diluted with water (5 mL) and cooled to 0-10° C. The reaction mixture pH was adjusted to ˜4-5 with 10% aqueous citric acid and extracted with EtOAc (2×30 mL). The combined organic layer was washed with brine (15 mL), separated, dried over Na₂SO₄, filtered and concentrated under reduced pressure to afford (1S,2R,5R)-3-((6-(4-fluorophenoxy)pyridin-3-yl)sulfonyl)-8-((2-methoxyethoxy)carbonyl)-3,8-diazabicyclo[3.2.1]octane-2-carboxylic acid (270 mg, 0.53 mmol, 86% yield) as a brown sticky gum. This was used as such for the next step without further purification. MS (ESI): m/z 510.0 [M+H]⁺.

### Step 5

### Synthesis of (1S,2R,5R)-2-methoxyethyl 3-((6-(4-fluorophenoxy)pyridin-3-yl)sulfonyl)-2-(((tetrahydro-2H-pyran-2-yl)oxy)carbamoyl)-3,8-diazabicyclo[3.2.1]octane-8-carboxylate

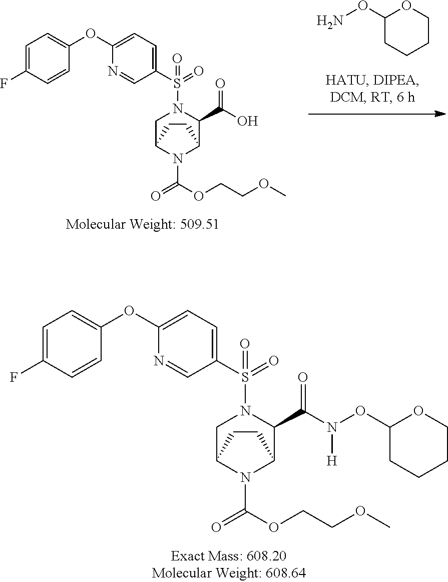 [SMILES: COCCOC(=O)N1[C@@H]2CC[C@H]1[C@H](C(=O)O)N(S(=O)(=O)c1ccc(Oc3ccc(F)cc3)nc1)C2 ; NOC1CCCCO1 ; COCCOC(=O)N1[C@@H]2CC[C@H]1[C@H](C(=O)NOC1CCCCO1)N(S(=O)(=O)c1ccc(Oc3ccc(F)cc3)nc1)C2]

To a stirred solution of (1S,2R,5R)-3-((6-(4-fluorophenoxy)pyridin-3-yl)sulfonyl)-8-((2-methoxyethoxy)carbonyl)-3,8-diazabicyclo[3.2.1]octane-2-carboxylic acid (250 mg, 0.49 mmol) in anhydrous DMF (6 mL) at 0-10° C., was added HATU (223 mg, 0.58 mmol) and DIPEA (0.44 mL, 2.4 mmol). The reaction mixture was stirred for 30 min at the same temperature and O-(tetrahydro-2H-pyran-2-yl)hydroxylamine (69 mg, 0.63 mmol) was added. The reaction mixture was allowed to warm to RT over a period of 30 min and stirred for 6 h while being monitored by TLC. The reaction mixture was quenched with ice cold water (15 mL) at 0° C. and extracted with EtOAc (2×30 mL). The combined organic layer was washed with brine (15 mL), separated, dried over anhydrous Na₂SO₄, filtered and concentrated under reduced pressure to afford (1S,2R,5R)-2-methoxyethyl 3-((6-(4-fluorophenoxy)pyridin-3-yl)sulfonyl)-2-(((tetrahydro-2H-pyran-2-yl)oxy)carbamoyl)-3,8-diazabicyclo[3.2.1]octane-8-carboxylate (200 mg, 0.33 mmol, 67% yield) as a colorless liquid. This was used as such for the next step without further purification. MS (ESI): m/z 606.9 [M−H]⁻.

### Step 6

### Synthesis of (1S,2R,5R)-2-methoxyethyl 3-((6-(4-fluorophenoxy)pyridin-3-yl)sulfonyl)-2-(hydroxycarbamoyl)-3,8-diazabicyclo[3.2.1]octane-8-carboxylate (Example 1)

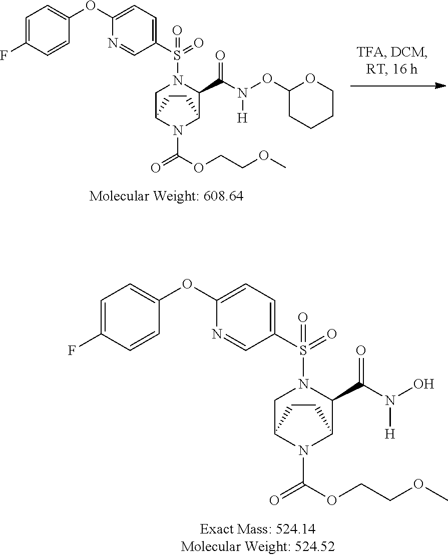 [SMILES: COCCOC(=O)N1[C@@H]2CC[C@H]1[C@H](C(=O)NOC1CCCCO1)N(S(=O)(=O)c1ccc(Oc3ccc(F)cc3)nc1)C2 ; COCCOC(=O)N1[C@@H]2CC[C@H]1[C@H](C(=O)NO)N(S(=O)(=O)c1ccc(Oc3ccc(F)cc3)nc1)C2]

To a stirred solution of (1S,2R,5R)-2-methoxyethyl 3-((6-(4-fluorophenoxy)pyridin-3-yl)sulfonyl)-2-(((tetrahydro-2H-pyran-2-yl)oxy)carbamoyl)-3,8-diazabicyclo[3.2.1]octane-8H-carboxylate (190 mg, 0.31 mmol) in DCM (4 mL) at 0-10° C., was added trifluoroacetic acid (0.24 mL, 3.1 mmol) dropwise. The reaction mixture was stirred at RT for 16 h while being monitored by TLC. The volatiles were evaporated, the residue was diluted with DCM (40 mL) and washed with saturated NaHCO₃ solution (2×10 mL). The organic layer was separated, dried over anhydrous Na₂SO₄, filtered, and concentrated under reduced pressure to afford 150 mg of crude compound. The crude was purified by RP prep HPLC (Table 2, Method 2) and fractions were lyophilized to afford (1S,2R,5R)-2-methoxyethyl 3-((6-(4-fluorophenoxy)pyridin-3-yl)sulfonyl)-2-(hydroxycarbamoyl)-3, 8-diazabicyclo[3.2.1]octane-8-carboxylate (Example 1) (45 mg, 0.08 mmol, 14% yield) as an off-white solid. MS (ESI): m/z 525.0 [M+H]; ¹H NMR (400 MHz, DMSO-d₆): δ 10.64 (brs, 1H), 8.76 (brs, 1H), 8.42 (d, J=2.4 Hz, 1H), 8.09 (dd, J=2.8, 8.8 Hz, 1H), 7.31-7.27 (m, 4H), 7.18 (d, J=8.8 Hz, 1H), 4.42-4.38 (m, 1H), 4.28-4.20 (m, 1H), 4.10-4.04 (m, 2H), 3.90-3.80 (m, 1H), 3.59-3.40 (m, 4H), 3.23 (s, 3H), 1.93-1.70 (m, 2H), 1.56-1.47 (in, 2H).

Similarly, below examples were prepared by following the same or analogous process as described in Example 1.

TABLE 4

| Ex. | Structure | Name | Analytical data |
|---|---|---|---|
| 2 | 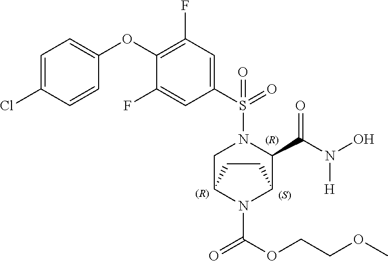 | (1S,2R,5R)-2- methoxyethyl 3-((4- (4-chlorophenoxy)- 3,5-difluoro- phenyl)sulfonyl)-2- (hydroxycarbamoyl)- 3,8-diazabicyclo- [3.2.1 ]octane-8- carboxylate | ¹H NMR at 90° C. (400 MHZ, DMSO-d₆): δ 10.36 (brs, 1H), 8.68 (brs, 1H), 7.60 (d, J = 7.2 Hz, 2H), 7.39 (d, J = 8.8 Hz, 2H), 7.06 (d, J = 8.8 Hz, 2H), 4.60-4.50 (m, 1H), 4.31 (d, J = 5.4 Hz, 1H), 4.28-4.12 (m, 1H), 4.10 -3.98 (m, 2H), 3.71- 3.67 (m, 1H), 3.54-3.43 (m, 3H), 3.25 (s, 3H), 1.97 - 1.79 (m, 2H), 1.68-1.56 (m, 2H); MS (ESI): m/z 575.9 [M + H]^{+.} |
| 3 | 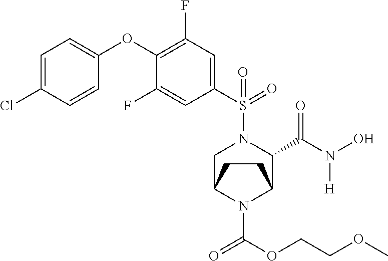 [SMILES: COCCOC(=O)N1[C@H]2CC[C@@H]1[C@@H](C(=O)NO)N(S(=O)(=O)c1cc(F)c(Oc3ccc(Cl)cc3)c(F)c1)C2] | 2-methoxyethyl (1R,2S,5S)-3-((4-(4- chlorophenoxy)-3,5- difluorophenyl)- sulfonyl)-2- (hydroxycarbamoyl)- 3,8-diaza- bicyclo[3.2.1]octane- 8-carboxylate | ¹H NMR (400 MHZ, DMSO- d₆): δ 10.64 (brs, 1H), 8.78 (brs, 1H), 7.71-7.66 (m, 2H), 7.45-7.39 (m, 2H), 7.12 - 7.04 (m, 2H), 4.50-4.40 (m, 1H), 4.32-4.27 (m, 1H), 4.10- 4.00 (m, 2H), 3.94-3.84 (m, 1H), 3.70-3.60 (m, 1H), 3.56-3.44 (m, 3H), 3.23 (s, 3H), 2.00-1.78 (m, 2H), 1.62- 1.48 (m, 2H); MS (ESI): m/z 576.0 [M + H]^{+.} |
| 4 | 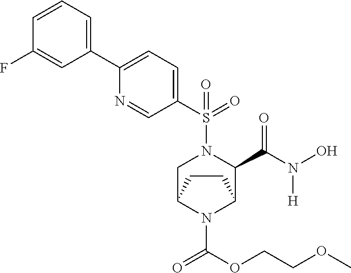 [SMILES: COCCOC(=O)N1[C@@H]2CC[C@H]1[C@H](C(=O)NO)N(S(=O)(=O)c1ccc(-c3cccc(F)c3)nc1)C2] | 2-methoxyethyl (1S,2R,5R)-3-((6-(3- fluorophenyl)pyridin- 3-yl)sulfonyl)-2- (hydroxycarbamoyl)- 3,8-diaza- bicyclo[3.2.1 ]octane- 8-carboxylate | ¹H NMR (400 MHZ, DMSO- d₆): δ 10.67 (brs, 1H), 8.92 (s, 1H), 8.77 (brs, 1H), 8.25 - 8.15 (m, 2H), 8.06-7.97 (m, 2H), 7.63-7.57 (m, 1H), 7.40- 7.35 (m, 1H), 4.50-4.40 (m, 1H), 4.33-4.26 (m, 1H), 4.18-3.99 (m, 2H), 3.92- 3.80 (m, 1H), 3.66-3.40 (m, 4H), 3.22 (s, 3H), 2.04-1.77 (m, 2H), 1.62-1.48 (m, 2H); MS (ESI): m/z 506.9 [M − H]^{-.} |
| 5 | 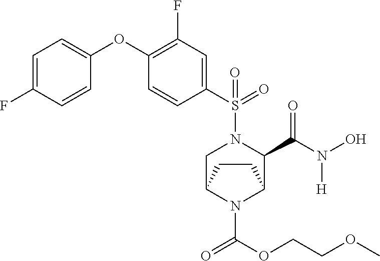 [SMILES: COCCOC(=O)N1[C@@H]2CC[C@H]1[C@H](C(=O)NO)N(S(=O)(=O)c1ccc(Oc3ccc(F)cc3)c(F)c1)C2] | 2-methoxyethyl (1S,2R,5R)-3-((3- fluoro-4-(4-fluoro- phenoxy)phenyl)- sulfonyl)-2- (hydroxycarbamoyl)- 3,8- diazabicyclo[3.2.1]- octane-8-carboxylate | ¹H NMR (400 MHZ, DMSO- d₆): δ 10.63 (brs, 1H), 8.76 (brs, 1H), 7.73 (d, J = 10.0 Hz, 1H), 7.53 (d, J = 8.4 Hz, 1H), 7.33-7.26 (m, 2H), 7.24- 7.17 (m, 2H), 7.11 (t, J = 8.4 Hz, 1H), 4.50-4.38 (m, 1H), 4.32-4.21 (m, 1H), 4.10 - 3.99 (m, 2H), 3.93-3.80 (m, 1H), 3.68-3.58 (m, 1H), 3.53- 3.40 (m, 3H), 3.23 (s, 3H), 2.00-1.70 (m, 2H), 1.56 - 1.40 (m, 2H); MS (ESI): m/z 542.0 [M + H]^{+.} |
| 6 | 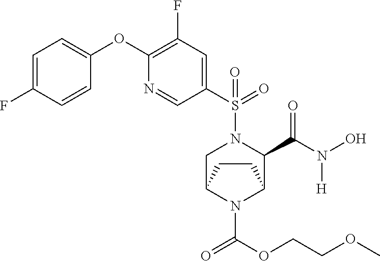 [SMILES: COCCOC(=O)N1[C@@H]2CC[C@H]1[C@H](C(=O)NO)N(S(=O)(=O)c1cnc(Oc3ccc(F)cc3)c(F)c1)C2] | 2-methoxyethyl (1S,2R,5R)-3-((5- fluoro-6-(4-fluoro- phenoxy)pyridin-3- yl)sulfonyl)-2- (hydroxycarbamoyl)- 3,8- diazabicyclo[3.2.1]- octane-8-carboxylate | ¹H NMR (400 MHZ, DMSO- d₆): δ 10.67 (brs, 1H), 8.80 (brs, 1H), 8.22 (s, 1H), 8.14 (d, J = 9.2 Hz, 1H), 7.34 - 7.26 (m, 4H), 4.50-4.38 (m, 1H), 4.32-4.23 (m, 1H), 4.12- 3.99 (m, 2H), 3.96-3.80 (m, 1H), 3.63-3.54 (m, 1H), 3.53-3.40 (m, 3H), 3.23 (s, 3H), 2.04-1.78 (m, 2H), 1.64- 1.50 (m, 2H); MS (ESI): m/z 543.3 [M + H]^{+.} |
| 7 | 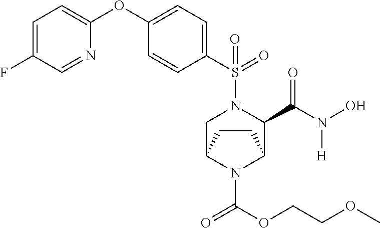 [SMILES: COCCOC(=O)N1[C@@H]2CC[C@H]1[C@H](C(=O)NO)N(S(=O)(=O)c1ccc(Oc3ccc(F)cn3)cc1)C2] | 2-methoxyethyl (1S,2R,5R)-3-((4- ((5-fluoropyridin-2- yl)oxy)phenyl)- sulfonyl)-2- (hydroxycarbamoyl)- 3,8-diazabicyclo- [3.2.1]octane-8- carboxylate | ¹H NMR (400 MHZ, DMSO- d₆): δ 10.62 (brs, 1H), 8.78 (brs, 1H), 8.25 (d, J = 2.8 Hz, 1H), 7.94-7.87 (m, 1H), 7.77 (d, J = 8.8 Hz, 2H), 7.30 - 7.21 (m, 3H), 4.50-4.37 (m, 1H), 4.31-4.20 (m, 1H), 4.11- 3.99 (m, 2H), 3.95-3.85 (m, 1H), 3.70-3.58 (m, 1H), 3.54-3.43 (m, 3H), 3.23 (s, 3H), 1.98-1.69 (m, 2H), 1.56- 1.36 (m, 2H); MS (ESI): m/z 525.3 [M + H]^{+.} |
| 8 | 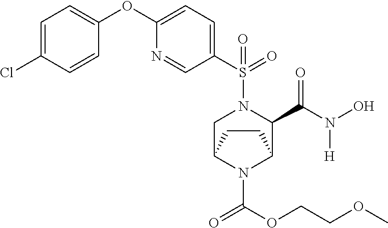 [SMILES: COCCOC(=O)N1[C@@H]2CC[C@H]1[C@H](C(=O)NO)N(S(=O)(=O)c1ccc(Oc3ccc(Cl)cc3)nc1)C2] | 2-methoxyethyl (1S,2R,5R)-3-((6-(4- chlorophenoxy)pyrid in-3-yl)sulfonyl)-2- (hydroxycarbamoyl)- 3,8-diazabicyclo- [3.2.1]octane-8- carboxylate | ¹H NMR (400 MHZ, DMSO- d₆): δ 10.63 (brs, 1H), 8.77 (brs, 1H), 8.43 (s, 1H), 8.10 (dd, J = 2.4, 8.8 Hz, 1H), 7.53- 7.48 (m, 2H), 7.28-7.20 (m, 3H), 4.50-4.38 (m, 1H), 4.32-4.23 (m, 1H), 4.12 - 3.99 (m, 2H), 3.94-3.80 (m, 1H), 3.62-3.38 (m, 4H), 3.23 (s, 3H), 2.02-1.76 (m, 2H), 1.68-1.46 (m, 2H); MS (ESI): m/z 539.1 [M − H]⁻ |
| 9 | 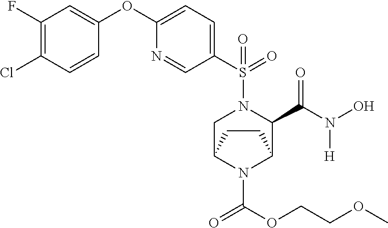 [SMILES: COCCOC(=O)N1[C@@H]2CC[C@H]1[C@H](C(=O)NO)N(S(=O)(=O)c1ccc(Oc3ccc(Cl)c(F)c3)nc1)C2] | 2-methoxyethyl (1S,2R,5R)-3-((6-(4- chloro-3-fluoro- phenoxy)pyridin-3- yl)sulfonyl)-2- (hydroxycarbamoyl)- 3,8-diaza- bicyclo[3.2.1 ]octane- 8-carboxylate | ¹H NMR (400 MHZ, DMSO- d₆): δ 10.70 (brs, 1H), 8.75 (brs, 1H), 8.45 (d, J = 1.6 Hz, 1H), 8.12 (dd, J = 2.4, 8.4 Hz, 1H), 7.67 (t, J = 8.8 Hz, 1H), 7.48 (dd, J = 2.4, 10.2 Hz, 1H), 7.26 (d, J = 8.8 Hz, 1H), 7.17-7.01 (m, 1H), 4.66 - 4.35 (m, 1H), 4.32-4.21 (m, 1H), 4.11-3.99 (m, 2H), 3.95- 3.80 (m, 1H), 3.61-3.36 (m, 4H), 3.23 (s, 3H), 2.00 - 1.75 (m, 2H), 1.70-1.47 (m, 2H); MS (ESI): m/z 559.1 [M + H]^{+.} |

The synthesis of 2-ethyl 8-(2-methoxyethyl) (1S,2S,5R)-3,8-diazabicyclo[3.2.1]octane-2,8-dicarboxylate (intermediate 2) is shown below.

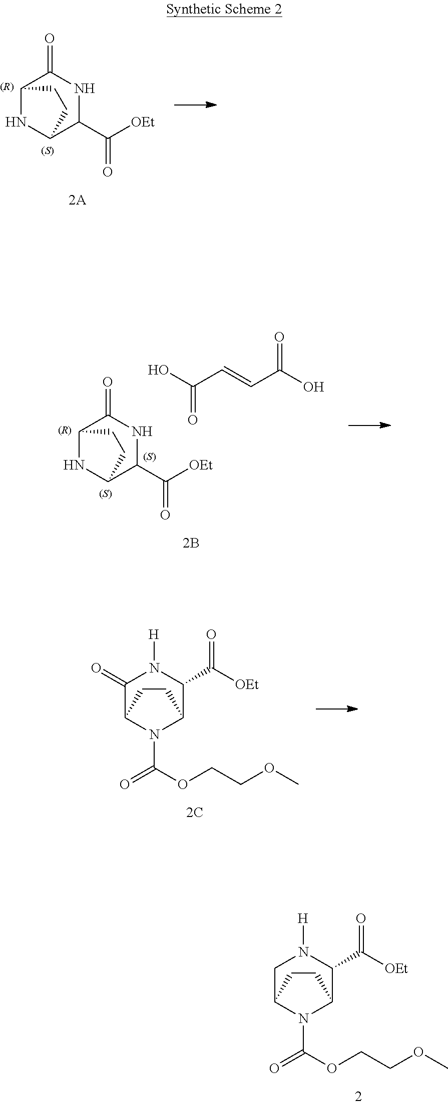

### Step 1

### Synthesis of ethyl (1S,2S,5R)-4-oxo-3,8-diazabicyclo[3.2.1]octane-2-carboxylate fumarate salt

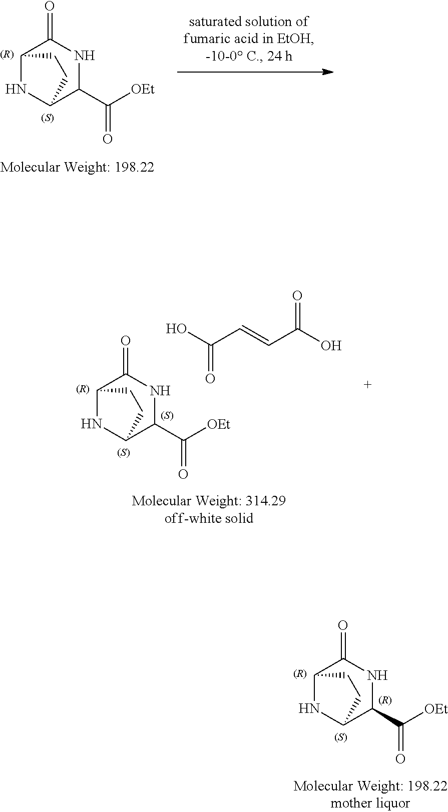

(1S,5R)-4-Oxo-3,8-diazabicyclo[3.2.1]octane-2-carboxylate (160 g, 0.81 mol) was dissolved in 2.3 L of saturated solution of fumaric acid in EtOH (90 g of fumaric acid was dissolved in 2.5 L of EtOH to make saturated solution) and transferred into a 5 L multi-neck round bottom flask fitted with mechanical stirrer at room temperature. The reaction mixture was cooled to −10° C.-0° C. and solid precipitation was observed after 30 min of stirring. The mixture was stirred for 24 h and filtered. The obtained light yellow solid compound was washed with diethyl ether (600 mL) and dried under high vacuum to afford ethyl (1S,2S,5R)-4-oxo-3,8-diazabicyclo[3.2.1]octane-2-carboxylate fumarate salt (60 g, 0.19 mol, 25% for 2 steps) as an off-white solid. ¹H NMR (400 MHz, CDCl₃): δ 8.18 (brs, 3H), 7.32 (s, 1H), 6.61 (s, 2H), 4.27 (d, J=4.4 Hz, 1H), 4.18-4.11 (m, 2H), 3.80-3.77 (m, 1H), 3.45-3.43 (m, 1H), 1.86-1.69 (m, 3H), 1.51-1.45 (m, 1H), 1.21 (t, J=7.2 Hz, 3H).

### Step 2

### Synthesis of 2-ethyl 8-(2-methoxyethyl) (1S,2S,5R)-4-oxo-3,8-diazabicyclo[3.2.1]octane-2,8-dicarboxylate

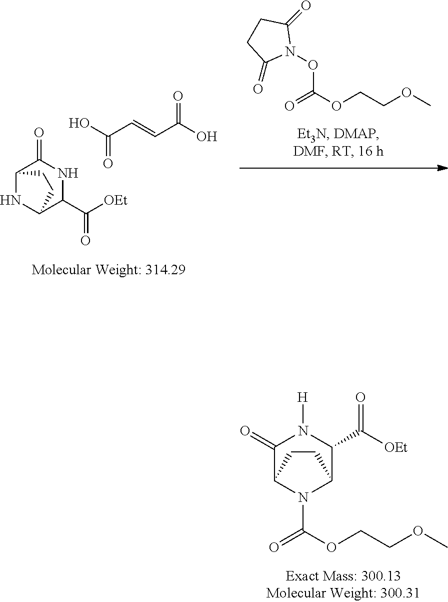

To a stirred solution of ethyl (1S,2S,5R)-4-oxo-3,8-diazabicyclo[3.2.1]octane-2-carboxylate fumarate salt (10 g, 31.8 mmol) in DMF (100 mL) at 0-10° C. was added Et₃N (22.2 mL, 159.1 mmol) and stirred for 10 min. 2,5-Dioxopyrrolidin-1-yl (2-methoxyethyl) carbonate (13.8 g, 63.6 mmol) was added to the reaction mixture at 0-10° C. and then DMAP (0.39 g, 3.18 mmol). The reaction was warmed to RT and stirred for 16 h while being monitored by TLC. The reaction was diluted with cold water (100 mL) and extracted with EtOAc (500 mL). The organic layer was washed with cold water (2×100 mL). The organic layer was dried over anhydrous Na₂SO₄, filtered and concentrated under reduced pressure to afford 2-ethyl 8-(2-methoxyethyl) (1S,2S,5R)-4-oxo-3,8-diazabicyclo[3.2.1]octane-2,8-dicarboxylate (7.3 g, 24.3 mmol, 76% yield) as a red syrup. This was used as such for the next step without further purification. ¹H NMR (400 MHz, CDCl₃): δ 5.87 (brs, 1H), 4.90-4.84 (m, 1H), 4.60-4.51 (m, 2H), 4.35-4.20 (m, 4H), 3.65-3.58 (m, 2H), 3.38 (s, 3H), 2.19-2.07 (m, 3H), 1.72-1.67 (m, 1H), 1.33 (t, J=7.2 Hz, 3H).

### Step 3

### Synthesis of 2-ethyl 8-(2-methoxyethyl) (1S,2S,5R)-3,8-diazabicyclo[3.2.1]octane-2,8-dicarboxylate (intermediate 2)

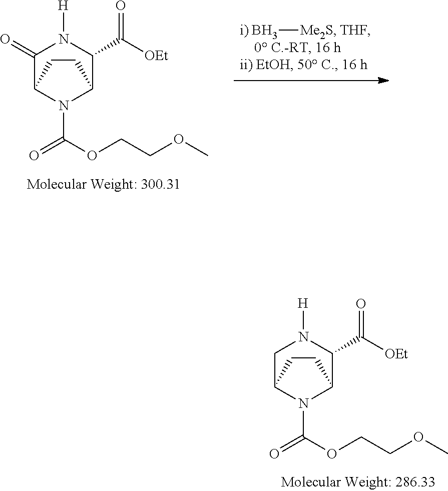

To a stirred solution of 2-ethyl 8-(2-methoxyethyl) (1S,2S,5R)-4-oxo-3,8-diazabicyclo[3.2.1]octane-2,8-dicarboxylate (7.3 g, 24.3 mmol) in dry THF (73 mL) at 0-10° C. was added BH₃·DMS (10 M solution, 14.6 mL, 146.0 mmol) drop wise. The reaction was slowly warmed to RT and stirred for 16 h while being monitored by TLC. The reaction was cooled to 0-10° C. and added EtOH (60 mL) dropwise. The reaction was stirred at 50° C. for 16 h and volatiles were distilled off under reduced pressure to afford 2-ethyl 8-(2-methoxyethyl) (1S,2S,5R)-3,8-diazabicyclo[3.2.1]octane-2,8-dicarboxylate (intermediate 2) (7.0 g, 24.4 mmol) as a colorless viscous liquid. This was used as such for the next step without further purification. ¹H NMR (400 MHz, CDCl₃): δ 4.53-4.49 (m, 1H), 4.30-4.18 (m, 4H), 3.93-3.80 (m, 4H), 3.63-3.59 (m, 2H), 3.38 (s, 3H), 1.31-1.20 (m, 4H), 1.17 (t, J=7.2 Hz, 3H).

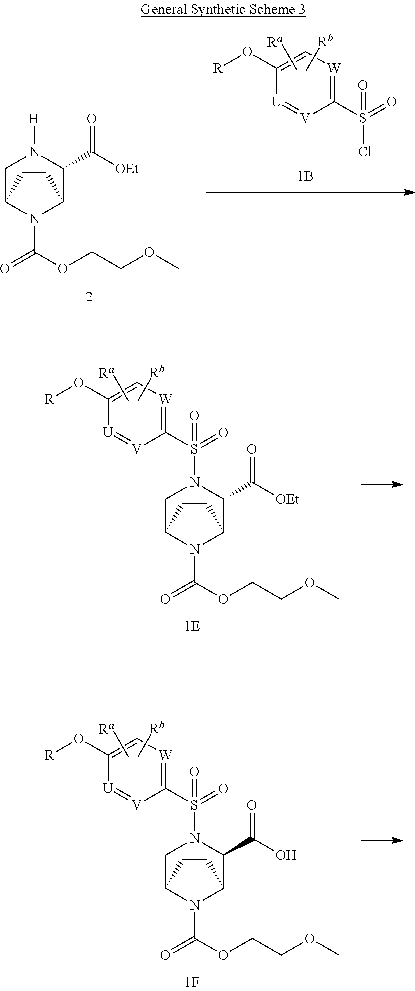

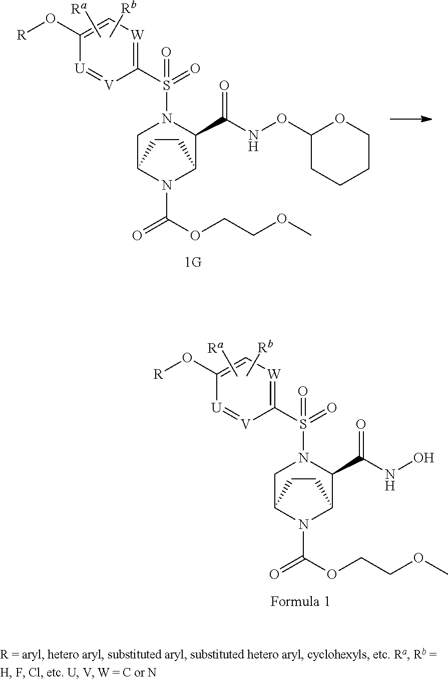

### Example 10

### Synthesis of 2-methoxyethyl (1S,2R,5R)-3-((6-(cyclohexyloxy)pyridin-3-yl)sulfonyl)-2-(hydroxycarbamoyl)-3,8-diazabicyclo[3.2.1]octane-8-carboxylate

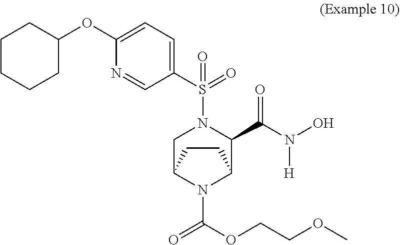 [SMILES: COCCOC(=O)N1[C@@H]2CC[C@H]1[C@H](C(=O)NO)N(S(=O)(=O)c1ccc(OC3CCCCC3)nc1)C2]

### Step 1

### Synthesis of 2-ethyl 8-(2-methoxyethyl) (1S,2S,5R)-3-((6-(cyclohexyloxy)pyridin-3-yl)sulfonyl)-3,8-diazabicyclo[3.2.1]octane-2,8-dicarboxylate

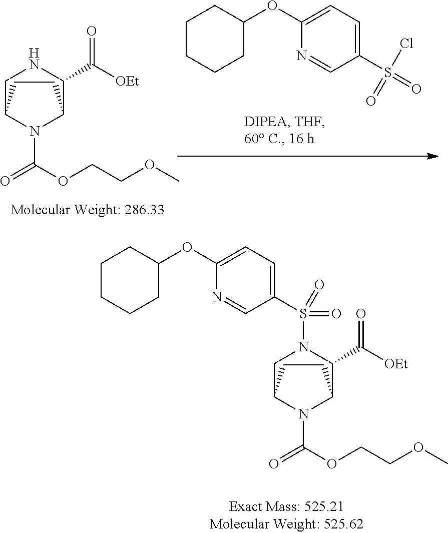 [SMILES: CCOC(=O)[C@H]1NC[C@H]2CC[C@@H]1N2C(=O)OCCOC ; O=S(=O)(Cl)c1ccc(OC2CCCCC2)nc1 ; CCOC(=O)[C@@H]1[C@@H]2CC[C@H](CN1S(=O)(=O)c1ccc(OC3CCCCC3)nc1)N2C(=O)OCCOC]

To a stirred solution of 2-ethyl 8-(2-methoxyethyl) (1S,2S,5R)-3,8-diazabicyclo[3.2.1]octane-2,8-dicarboxylate (250 mg, 0.87 mmol) in THF (5 mL) under nitrogen at 0-10° C. was added DIPEA (0.45 mL, 2.6 mmol) and stirred for 20 min. 6-(Cyclohexyloxy)-pyridine-3-sulfonyl chloride (360 mg, 1.31 mmol) was dissolved in THF (5 mL) and added to the reaction mixture at the same temperature. The reaction mixture was stirred at 60° C. for 16 h while being monitored by TLC. The reaction mixture was cooled to RT, diluted with water (10 mL) and extracted with ethyl acetate (2×20 mL). The combined organic layer was dried over anhydrous Na₂SO₄, filtered and concentrated under reduced pressure to obtain crude compound. The crude product was purified by silica gel column chromatography (60-120 mesh, 15-40% EtOAc in hexanes) to afford 2-ethyl 8-(2-methoxyethyl) (1S,2S,5R)-3-((6-(cyclohexyloxy)pyridin-3-yl)sulfonyl)-3,8-diazabicyclo[3.2.1]octane-2,8-dicarboxylate (240 mg, 0.45 mmol, 52% yield) as a yellow gum. MS (ESI): m/z 526.2 [M+H]⁺.

### Step 2

### Synthesis of (1S,2R,5R)-3-((6-(cyclohexyloxy)pyridin-3-yl)sulfonyl)-8-((2-methoxyethoxy)carbonyl)-3,8-diazabicyclo[3.2.1]octane-2-carboxylic acid

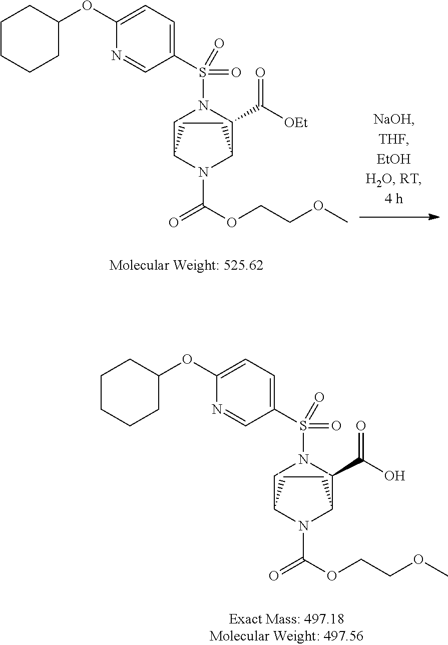 [SMILES: CCOC(=O)[C@@H]1[C@@H]2CC[C@H](CN1S(=O)(=O)c1ccc(OC3CCCCC3)nc1)N2C(=O)OCCOC ; COCCOC(=O)N1[C@@H]2CC[C@H]1[C@H](C(=O)O)N(S(=O)(=O)c1ccc(OC3CCCCC3)nc1)C2]

Similar procedure was followed as described for Step 4 of Example 1, to obtain (1S,2R,5R)-3-((6-(cyclohexyloxy)pyridin-3-yl)sulfonyl)-8-((2-methoxyethoxy)carbonyl)-3,8-diazabicyclo[3.2.1]octane-2-carboxylic acid (150 mg, 0.30 mmol, 66% yield) as a brown gum. This was used as such for the next step without further purification. MS (ESI): m/z 498.2 [M+H]⁺.

### Step 3

### Synthesis of 2-methoxyethyl (1S,2R,5R)-3-((6-(cyclohexyloxy)pyridin-3-yl)sulfonyl)-2-(((tetrahydro-2H-pyran-2-yl)oxy)carbamoyl)-3,8-diazabicyclo[3.2.1]octane-8-carboxylate

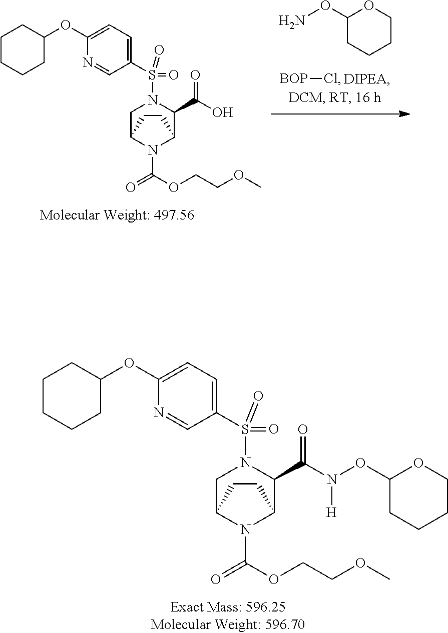

To a stirred solution of (1S,2R,5R)-3-((6-(cyclohexyloxy)pyridin-3-yl)sulfonyl)-8-((2-methoxyethoxy)carbonyl)-3,8-diazabicyclo[3.2.1]octane-2-carboxylic acid (150 mg, 0.30 mmol) in DCM (3 mL) at 0-10° C. was added BOP—Cl (191 mg, 0.75 mmol) and DIPEA (0.16 mL, 0.9 mmol). The reaction was stirred for 30 min and O-(tetrahydro-2H-pyran-2-yl)hydroxylamine (46 mg, 0.39 mmol) was added. The reaction mixture was allowed to warm to RT over a period of 30 min and stirred for 16 h while being monitored by TLC. The reaction was quenched with ice cold water (20 mL) at 0° C. and the aqueous layer was extracted with DCM (3×20 mL). The combined organic layer was washed with brine (20 ML), dried over anhydrous Na₂SO₄, filtered and concentrated under reduced pressure to afford 2-methoxyethyl (1S,2R,5R)-3-((6-(cyclohexyloxy)pyridin-3-yl)sulfonyl)-2-(((tetrahydro-2H-pyran-2-yl)oxy)carbamoyl)-3,8-diazabicyclo[3.2.1]octane-8-carboxylate (180 mg, 0.30 mmol) as a yellow gum. This was used as such for the next step without further purification. MS (ESI): m/z 595.3 [M−H]⁻.

### Step 4

### Synthesis of 2-methoxyethyl (1S,2R,5R)-3-((6-(cyclohexyloxy)pyridin-3-yl)sulfonyl)-2-(hydroxycarbamoyl)-3,8-diazabicyclo[3.2.1]octane-8-carboxylate (Example 10)

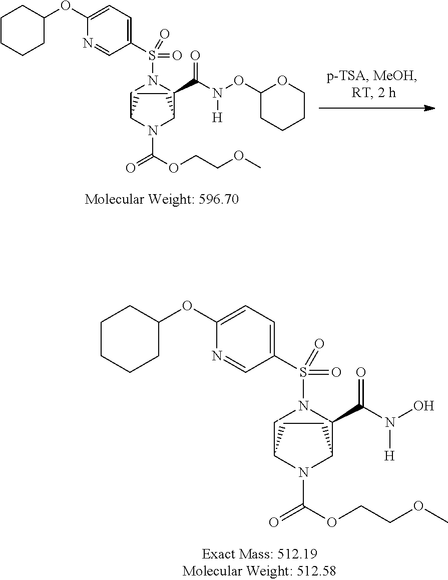 [SMILES: COCCOC(=O)N1[C@@H]2CC[C@H]1[C@H](C(=O)NOC1CCCCO1)N(S(=O)(=O)c1ccc(OC3CCCCC3)nc1)C2 ; COCCOC(=O)N1[C@@H]2CC[C@H]1[C@H](C(=O)NO)N(S(=O)(=O)c1ccc(OC3CCCCC3)nc1)C2]

To a stirred solution of 2-methoxyethyl (1S,2R,5R)-3-((6-(cyclohexyloxy)pyridin-3-yl)sulfonyl)-2-(((tetrahydro-2H-pyran-2-yl)oxy)carbamoyl)-3,8-diazabicyclo[3.2.1]octane-8-carboxylate (170 mg. 0.28 mmol) in MeOH (4 mL) at 0-10° C. was added p-TSA (271 mg, 1.42 mmol). The reaction was stirred at RT for 2 h while being monitored by TLC. The reaction was quenched with aqueous saturated NaHCO₃ solution (5 mL) and was extracted with DCM (3×20 mL). The organic layer was separated, dried over anhydrous Na₂SO₄, filtered, and concentrated under reduced pressure to afford crude compound. The crude compound was purified by RP prep HPLC (Table 2, Method 3) and fractions were lyophilized to afford 2-methoxyethyl (1S,2R,5R)-3-((6-(cyclohexyloxy)pyridin-3-yl)sulfonyl)-2-(hydroxycarbamoyl)-3, 8-diazabicyclo[3.2.1]octane-8-carboxylate (Example 10) (38.4 mg, 0.075 mmol, 26% yield for 2 steps) as an off-white solid. MS (ESI): m/z 513.2 [M+H]⁺; ¹H NMR (400 MHz, DMSO-d₆): δ 10.64 (brs, 1H), 8.78 (brs, 1H), 8.46 (d, J=2.4 Hz, 1H), 7.91 (dd, J=2.4, 8.8 Hz, 1H), 6.89 (d, J=8.8 Hz, 1H), 5.11-5.02 (m, 1H), 4.70-4.35 (m, 1H), 4.31-4.20 (m, 1H), 4.12-3.98 (m, 2H), 3.91-3.78 (m, 1H), 3.61-3.35 (m, 4H), 3.23 (s, 3H), 2.00-1.89 (m, 2H), 1.89-1.75 (m, 2H), 1.75-31.69 (m, 2H), 1.60-1.30 (m, 8H).

Similarly, below examples were prepared by following the same or analogous process as described in Example 10.

TABLE 5

| Ex. | Structure | Name | Analytical data |
|---|---|---|---|
| 11 | 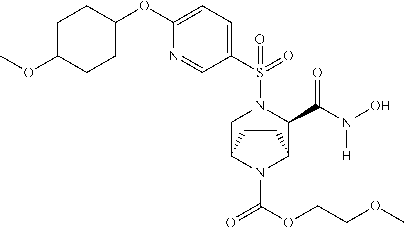 | 2-methoxyethyl (1S,2R,5R)-2- (hydroxycarbamoyl)- 3-((6-((4-methoxy- cyclohexyl)oxy)- pyridin-3-yl)- sulfonyl)-3,8-diaza- bicyclo[3.2.1]octane- 8-carboxylate | ¹H NMR (400 MHZ, DMSO- d₆): δ 10.67 (brs, 1H), 8.78 (brs, 1H), 8.46 (d, J = 2.4 Hz, 1H), 7.92 (dd, J = 2.4, 8.8 Hz, 1H), 6.91-6.88 (m, 1H), 5.18- 5.03 (m, 1H), 4.68-4.35 (m, 1H), 4.31-4.20 (m, 1H), 4.11-3.99 (m, 2H), 3.91 - 3.80 (m, 1H), 3.61-3.35 (m, 5H), 3.23 (s, 3H), 3.22 (s, 3H), 2.10-1.90 (m, 3H), 1.90- 1.61 (m, 5H), 1.60-1.30 (m, 4H); MS (ESI): m/z 543.2 [M + H]^{+.} |
| 12 | 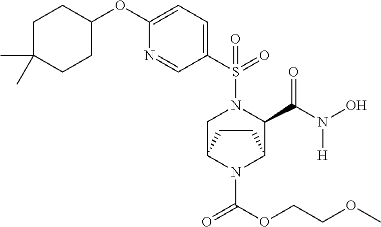 | 2-methoxyethyl (1S,2R,5R)-3-((6- ((4,4-dimethyl- cyclohexyl)- oxy)pyridin-3-yl)- sulfonyl)-2- (hydroxycarbamoyl)- 3,8- diazabicyclo[3.2.1]- octane-8-carboxylate | ¹H NMR (400 MHZ, DMSO- d₆): δ 10.64 (brs, 1H), 8.76 (brs, 1H), 8.45 (d, J = 2.4 Hz, 1H), 7.91 (dd, J = 2.4, 8.8 Hz, 1H), 6.89 (d, J = 8.8 Hz, 1H), 5.11-5.02 (m, 1H), 4.70 - 4.35 (m, 1H), 4.31-4.20 (m, 1H), 4.11-3.99 (m, 2H), 3.98- 3.79 (m, 1H), 3.61-3.35 (m, 4H), 3.23 (s, 3H), 2.00 - 1.70 (m, 4H), 1.70-1.58 (m, 2H), 1.58-1.60 (m, 4H), 1.32- 1.23 (m, 2H), 0.94 (d, J = 6.8 Hz, 6H); MS (ESI): m/z 541.2 [M + H]^{+.} |
| 13 | 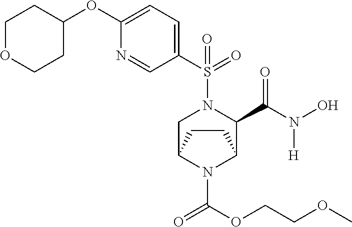 [SMILES: COCCOC(=O)N1[C@@H]2CC[C@H]1[C@H](C(=O)NO)N(S(=O)(=O)c1ccc(OC3CCOCC3)nc1)C2] | 2-methoxyethyl (1S,2R,5R)-2- (hydroxycarbamoyl)- 3-((6-((tetrahydro- 2H-pyran-4-yl)oxy)- pyridin-3- yl)sulfonyl)-3,8- diazabicyclo[3.2.1]- octane-8-carboxylate | ¹H NMR (400 MHZ, DMSO- d₆): δ 10.68 (brs, 1H), 8.78 (brs, 1H), 8.47 (d, J = 2.0 Hz, 1H), 7.94 (dd, J = 2.4, 8.8 Hz, 1H), 6.94 (d, J = 8.8 Hz, 1H), 5.30-5.21 (m, 1H), 4.68 - 4.35 (m, 1H), 4.30-4.20 (m, 1H), 4.13-3.98 (m, 2H), 3.93- 3.79 (m, 3H), 3.61-3.35 (m, 6H), 3.22 (s, 3H), 2.05 - 1.71 (m, 4H), 1.70-1.41 (m, 4H); MS (ESI): m/z 515.2 [M + H]^{+.} |

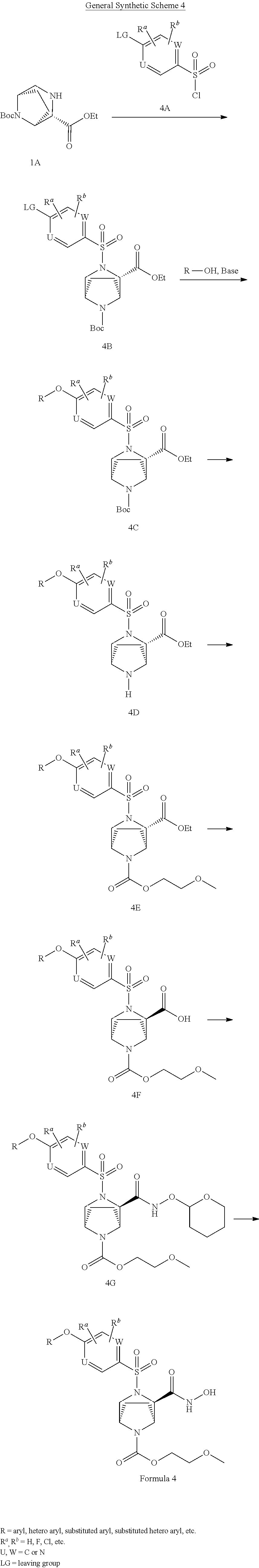

### Example 14

### Synthesis of 2-methoxyethyl (1S,2R,5R)-3-((5-(4-fluorophenoxy)pyridin-2-yl)sulfonyl)-2-(hydroxycarbamoyl)-3,8-diazabicyclo[3.2.1]octane-8-carboxylate

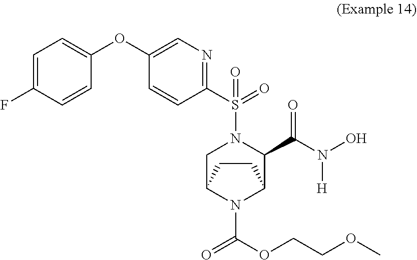 [SMILES: COCCOC(=O)N1[C@@H]2CC[C@H]1[C@H](C(=O)NO)N(S(=O)(=O)c1ccc(Oc3ccc(F)cc3)cn1)C2]

### Step 1

### Synthesis of 8-(tert-butyl) 2-ethyl (1S,2S,5R)-3-((5-fluoropyridin-2-yl)sulfonyl)-3,8-diazabicyclo[3.2.1]octane-2,8-dicarboxylate

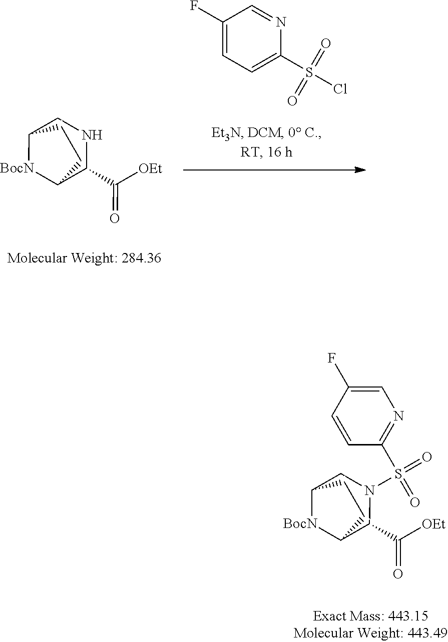 [SMILES: O=S(=O)(Cl)c1ccc(F)cn1 ; CCOC(=O)[C@H]1NC[C@H]2CC[C@@H]1N2C(=O)OC(C)(C)C ; CCOC(=O)[C@@H]1[C@@H]2CC[C@H](CN1S(=O)(=O)c1ccc(F)cn1)N2C(=O)OC(C)(C)C]

To a stirred solution of (1S,2S,5R)-8-tert-butyl 2-ethyl 3,8-diazabicyclo[3.2.1]octane-2,8-dicarboxylate (200 mg, 0.70 mmol) in DCM (5 mL) under nitrogen atmosphere at 0-10° C., was added Et₃N (0.2 mL, 1.40 mmol) and stirred for 20 min. 5-Fluoropyridine-2-sulfonyl chloride (205 mg, 1.05 mmol) was dissolved in DCM (2 mL) and added to the reaction mixture at same temperature. The reaction mixture was stirred at room temperature for 16 h while being monitored by TLC. The reaction mixture was diluted with water (20 mL) and extracted with DCM (2×20 mL). The combined organic layer was dried over Na₂SO₄, filtered and concentrated under reduced pressure to obtain crude product. The crude product was purified by silica gel column chromatography (100-200 mesh, 20% EtOAc in hexanes) to afford 8-(tert-butyl) 2-ethyl (1S,2S,5R)-3-((5-fluoropyridin-2-yl)sulfonyl)-3,8-diazabicyclo[3.2.1]octane-2,8-dicarboxylate (200 mg, 0.45 mmol, 64% yield) as an off-white solid. MS (ESI): m/z 444.4 [M+H]⁺.

### Step 2

### Synthesis of 8-(tert-butyl) 2-ethyl (1S,2S,5R)-3-((5-(4-fluorophenoxy)pyridin-2-yl)sulfonyl)-3,8-diazabicyclo[3.2.1]octane-2,8-dicarboxylate

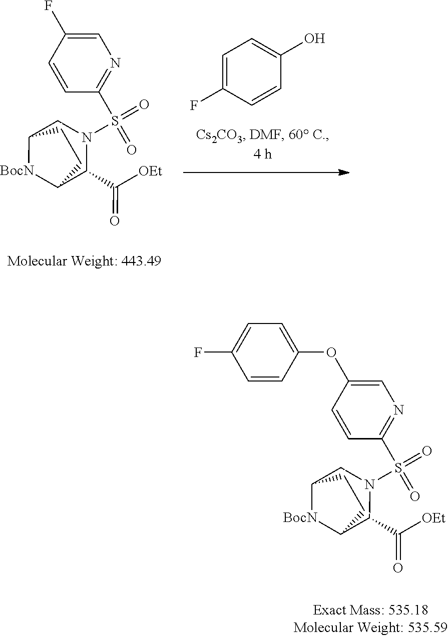 [SMILES: CCOC(=O)[C@@H]1[C@@H]2CC[C@H](CN1S(=O)(=O)c1ccc(F)cn1)N2C(=O)OC(C)(C)C ; Oc1ccc(F)cc1 ; CCOC(=O)[C@@H]1[C@@H]2CC[C@H](CN1S(=O)(=O)c1ccc(Oc3ccc(F)cc3)cn1)N2C(=O)OC(C)(C)C]

To a stirred solution of 8-(tert-butyl) 2-ethyl (1S,2S,5R)-3-((5-fluoropyridin-2-yl)sulfonyl)-3,8-diazabicyclo[3.2.1]octane-2,8-dicarboxylate (200 mg, 0.45 mmol) in DMF (3 mL), was added Cs₂CO₃ (219 mg, 0.67 mmol) followed by 4-fluorophenol (50 mg, 0.45 mmol) in DMF (1 mL) at RT and the mixture was stirred at 60° C. for 4 h while monitored by TLC. The reaction mixture was cooled to room temperature, quenched with ice cold water (10 mL) and extracted with EtOAc (2×10 mL). The combined organic layer was washed with brine (20 mL), dried over Na₂SO₄, filtered and concentrated under reduced pressure. The crude product was purified by silica gel column chromatography (100-200 mesh, 10% EtOAc in hexanes) to afford 8-(tert-butyl) 2-ethyl (1S,2S,5R)-3-((5-(4-fluorophenoxy)pyridin-2-yl)sulfonyl)-3,8-diazabicyclo[3.2.1]octane-2,8-dicarboxylate (170 mg, 0.31 mmol, 68% yield) as a colorless liquid. MS (ESI): m/z 536.2 [M+H]⁺.

### Step 3

### Synthesis of ethyl (1S,2S,5R)-3-((5-(4-fluorophenoxy)pyridin-2-yl)sulfonyl)-3,8-diazabicyclo[3.2.1]octane-2-carboxylate

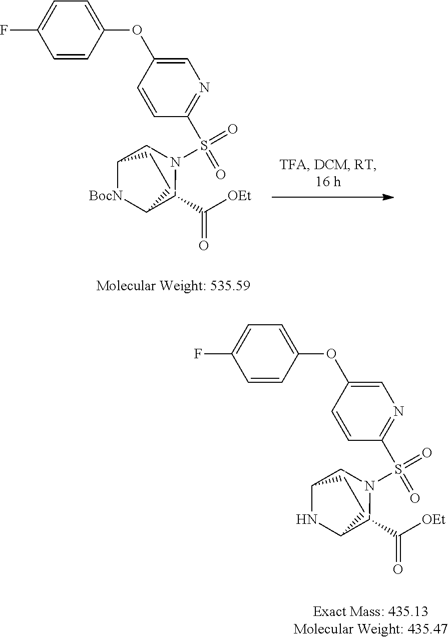 [SMILES: CCOC(=O)[C@@H]1[C@@H]2CC[C@H](CN1S(=O)(=O)c1ccc(Oc3ccc(F)cc3)cn1)N2C(=O)OC(C)(C)C ; CCOC(=O)[C@@H]1[C@@H]2CC[C@H](CN1S(=O)(=O)c1ccc(Oc3ccc(F)cc3)cn1)N2]

Similar procedure was followed as described for Step 2 of Example 1, to obtain ethyl (1S,2S,5R)-3-((5-(4-fluorophenoxy)pyridin-2-yl)sulfonyl)-3,8-diazabicyclo[3.2.1]octane-2-carboxylate (140 mg, 0.31 mmol, quantitative) as a colorless liquid. MS (ESI): m/z 436.2 [M+H]⁺.

### Step 4

### Synthesis of 2-ethyl 8-(2-methoxyethyl) (1S,2S,5R)-3-((5-(4-fluorophenoxy)pyridin-2-yl)sulfonyl)-3,8-diazabicyclo[3.2.1]octane-2,8-dicarboxylate

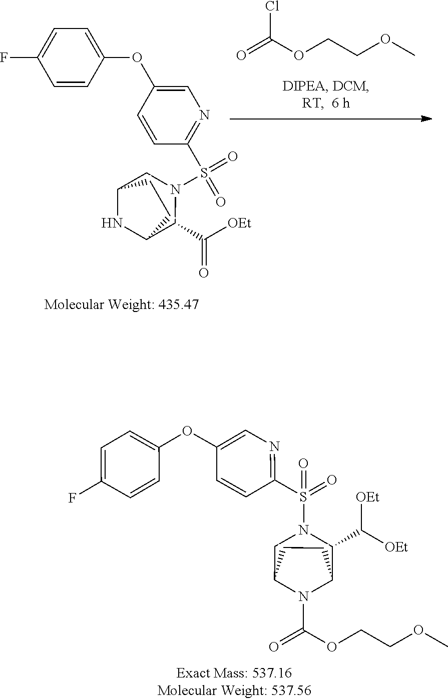

Similar procedure was followed as described for Step 3 of Example 1, to obtain 2-ethyl 8-(2-methoxyethyl) (1S,2S,5R)-3-((5-(4-fluorophenoxy)pyridin-2-yl)sulfonyl)-3,8-diazabicyclo[3.2.1]octane-2,8-dicarboxylate (150 mg, 0.27 mmol, 87% yield) as a colorless liquid. MS (ESI): m/z 538.2 [M+H]⁺.

### Step 5

### Synthesis of (1S,2R,5R)-3-((5-(4-fluorophenoxy)pyridin-2-yl)sulfonyl)-8-((2-methoxyethoxy)carbonyl)-3,8-diazabicyclo[3.2.1]octane-2-carboxylic acid

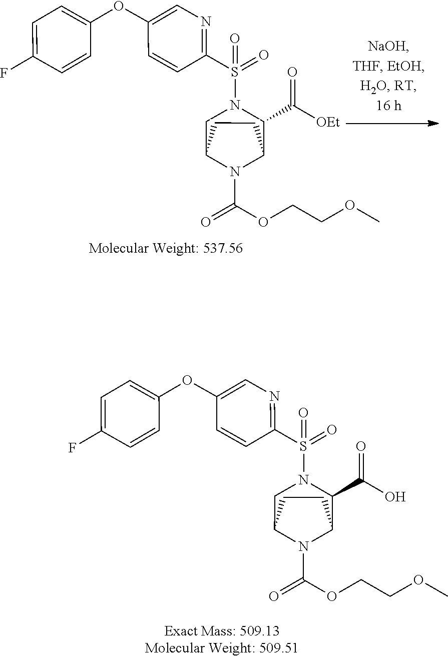 [SMILES: CCOC(=O)[C@@H]1[C@@H]2CC[C@H](CN1S(=O)(=O)c1ccc(Oc3ccc(F)cc3)cn1)N2C(=O)OCCOC ; COCCOC(=O)N1[C@@H]2CC[C@H]1[C@H](C(=O)O)N(S(=O)(=O)c1ccc(Oc3ccc(F)cc3)cn1)C2]

Similar procedure was followed as described for Step 4 of Example 1, to obtain (1S,2R,5R)-3-((5-(4-fluorophenoxy)pyridin-2-yl)sulfonyl)-8-((2-methoxyethoxy)carbonyl)-3,8-diazabicyclo[3.2.1]octane-2-carboxylic acid (150 mg, 0.28 mmol, quantitative) as a colorless sticky gum. MS (ESI): m/z 510.4 [M+H]⁺.

### Step 6

### Synthesis of 2-methoxyethyl (1S,2R,5R)-3-((5-(4-fluorophenoxy)pyridin-2-yl)sulfonyl)-2-(((tetrahydro-2H-pyran-2-yl)oxy)carbamoyl)-3,8-diazabicyclo[3.2.1]octane-8-carboxylate

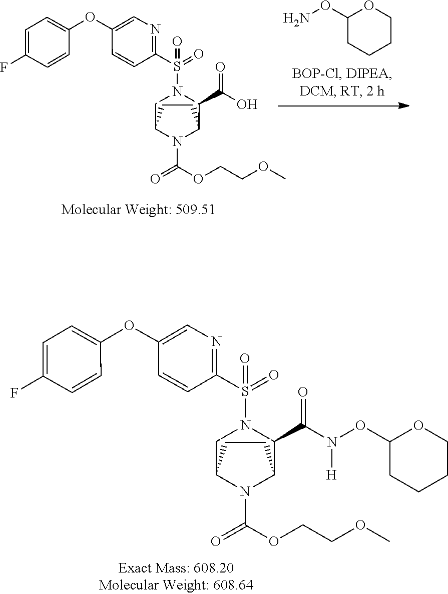 [SMILES: COCCOC(=O)N1[C@@H]2CC[C@H]1[C@H](C(=O)O)N(S(=O)(=O)c1ccc(Oc3ccc(F)cc3)cn1)C2 ; NOC1CCCCO1 ; COCCOC(=O)N1[C@@H]2CC[C@H]1[C@H](C(=O)NOC1CCCCO1)N(S(=O)(=O)c1ccc(Oc3ccc(F)cc3)cn1)C2]

To a stirred solution of (1S,2R,5R)-3-((5-(4-fluorophenoxy)pyridin-2-yl)sulfonyl)-8-((2-methoxyethoxy)carbonyl)-3,8-diazabicyclo[3.2.1]octane-2-carboxylic acid (80 mg, 0.15 mmol) in DCM (3 mL), was added BOP—Cl (100 mg, 0.39 mmol) and the mixture was stirred for 30 min at RT. The reaction mixture was cooled to 0-10° C., DIPEA (0.08 mL, 0.47 mmol) was added to it and stirred for 10 min then, 0-(tetrahydro-2H-pyran-2-yl)hydroxylamine hydrochloride (23 mg, 0.204 mmol) was added at the same temperature. The reaction mixture was allowed to warm to RT over a period of 30 min and stirred for 2 h while being monitored by TLC. The reaction mixture was quenched with ice cold water (20 mL) at 0° C. and the aqueous layer was extracted with DCM (3×20 mL). The combined organic layer was washed with brine (20 mL), dried over anhydrous Na₂SO₄, filtered and concentrated under reduced pressure to afford 2-methoxyethyl (1S,2R,5R)-3-((5-(4-fluorophenoxy)pyridin-2-yl)sulfonyl)-2-(((tetrahydro-2H-pyran-2-yl)oxy)carbamoyl)-3,8-diazabicyclo[3.2.1]octane-8-carboxylate (100 mg, 0.16 mmol, quantitative) as an off-white solid. This was used as such for the next step without further purification. MS (ESI): m/z 609 [M+H]⁺.

### Step 7

### Synthesis of 2-methoxyethyl (1S,2R,5R)-3-((5-(4-fluorophenoxy)pyridin-2-yl)sulfonyl)-2-(hydroxycarbamoyl)-3,8-diazabicyclo[3.2.1]octane-8-carboxylate (Example 14)

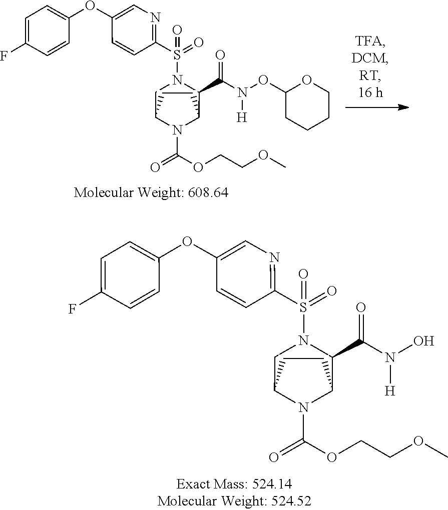 [SMILES: COCCOC(=O)N1[C@@H]2CC[C@H]1[C@H](C(=O)NOC1CCCCO1)N(S(=O)(=O)c1ccc(Oc3ccc(F)cc3)cn1)C2 ; COCCOC(=O)N1[C@@H]2CC[C@H]1[C@H](C(=O)NO)N(S(=O)(=O)c1ccc(Oc3ccc(F)cc3)cn1)C2]

Similar procedure was followed as described for Step 6 of Example 1 and purification was done by RP prep HPLC (Table 2, Method 3) to obtain 2-methoxyethyl (1S,2R,5R)-3-((5-(4-fluorophenoxy)pyridin-2-yl) sulfonyl)-2-(hydroxycarbamoyl)-3, 8-diazabicyclo[3.2.1]octane-8-carboxylate (Example 14) (10 mg, 0.019 mmol, 11% yield) as an off-white solid. MS (ESI): m/z 525.2 [M+H]⁺; ¹H NMR (400 MHz, DMSO-d₆): δ 10.60 (brs, 1H), 8.73 (brs, 1H), 8.49 (d, J=2.0 Hz, 1H), 7.84 (d, J=(8.8 Hz, 1H), 7.46 (dd, J=2.8, 8.8 Hz, 1H), 7.36-7.25 (m, 4H), 4.48-4.38 (m, 1H), 4.30-4.20 (m, 1H), 4.12-4.02 (m, 2H), 3.80-3.68 (m, 1H), 3.54-3.40 (m, 4H), 3.23 (s, 3H), 1.93-1.68 (m, 2H), 1.58-1.30 (in, 2H).

Similarly, below examples were prepared by following the same or analogous process as described in Example 14.

TABLE 6

| Ex. | Structure | Name | Analytical data |
|---|---|---|---|
| 15 | 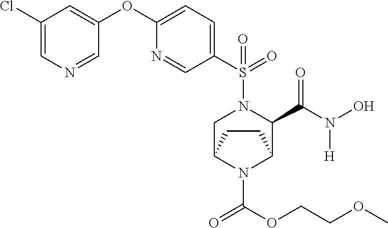 [SMILES: COCCOC(=O)N1[C@@H]2CC[C@H]1[C@H](C(=O)NO)N(S(=O)(=O)c1ccc(Oc3cncc(Cl)c3)nc1)C2] | (1S,2R,5R)-2- methoxyethyl 3-((6- ((5-chloropyridin-3- yl)oxy)pyridin-3-yl)- sulfonyl)-2- (hydroxycarbamoyl)- 3,8- diazabicyclo[3.2.1]- octane-8-carboxylate | ¹H NMR (400 MHZ, DMSO- d₆): δ 10.67 (brs, 1H), 8.76 (brs, 1H), 8.57 (d, J = 2.0 Hz, 1H), 8.53 (d, J = 6.8 Hz, 1H), 8.45 (s, 1H), 8.15 (dd, J = 2.4, 8.8 Hz, 1H), 8.06 (t, J = 2.8 Hz, 1H), 7.34 (d, J = 8.8 Hz, 1H), 4.48-4.38 (m, 1H), 4.30- 4.23 (m, 1H), 4.12-4.01 (m, 2H), 3.90-3.80 (m, 1H), 3.64-3.40 (m, 4H), 3.23 (s, 3H), 2.00-1.78 (m, 2H), 1.62- 1.48 (m, 2H); MS (ESI): m/z 542.1 [M + H]⁺. |
| 16 | 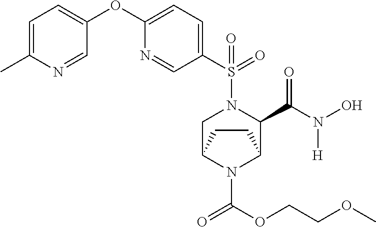 | 2-methoxyethyl (1S,2R,5R)-2- (hydroxycarbamoyl)- 3-((6-((6- methylpyridin-3-yl)- oxy)pyridin-3-yl)- sulfonyl)-3,8-diaza- bicyclo[3.2.1 ]octane- 8-carboxylate | ¹H NMR (400 MHZ, DMSO- d₆): δ 10.70 (brs, 1H), 8.77 (brs, 1H), 8.41 (s, 1H), 8.36 (d, J = 2.8 Hz, 1H), 8.11 (dd, J = 2.4, 8.8 Hz, 1H), 7.60 (dd, J = 2.4, 8.4 Hz, 1H), 7.35 (d, J = 8.8 Hz, 1H), 7.26 (d, J = 8.8 Hz, 1H), 4.48-4.38 (m, 1H), 4.30-4.23 (m, 1H), 4.12- 4.01 (m, 2H), 3.90-3.80 (m, 1H), 3.64-3.40 (m, 4H), 3.23 (s, 3H), 2.50 (s, 3H), 2.04- 1.76 (m, 2H), 1.62-1.48 (m, 2H); MS (ESI): m/z 522.2 [M + H]⁺. |
| 17 | 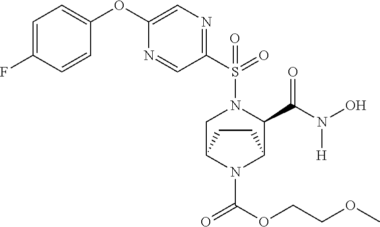 [SMILES: COCCOC(=O)N1[C@@H]2CC[C@H]1[C@H](C(=O)NO)N(S(=O)(=O)c1cnc(Oc3ccc(F)cc3)cn1)C2] | 2-methoxyethyl (1S,2R,5R)-3-((5-(4- fluorophenoxy)- pyrazin-2- yl)sulfonyl)-2- (hydroxycarbamoyl)- 3,8-diazabicyclo- [3.2.1]octane-8- carboxylate | ¹H NMR (400 MHZ, DMSO- d₆): δ 10.83 (brs, 1H), 8.94 (brs, 1H), 8.67 (s, 1H), 8.53 (s, 1H), 8.07 (s, 1H), 7.38- 7.30 (m, 4H), 4.32-4.28 (m, 1H), 4.17-3.96 (m, 5H), 3.53- 3.48 (m, 3H), 3.25 (s, 3H), 1.96-1.70 (m, 2H), 1.68- 1.40 (m, 2H); MS (ESI): m/z 526.1 [M + H]⁺. |
| 18 | 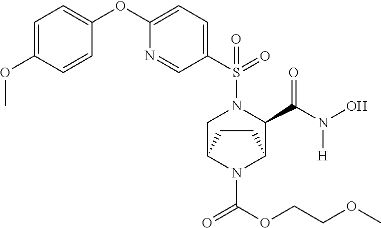 [SMILES: COCCOC(=O)N1[C@@H]2CC[C@H]1[C@H](C(=O)NO)N(S(=O)(=O)c1ccc(Oc3ccc(OC)cc3)nc1)C2] | 2-methoxyethyl (1S,2R,5R)-2- (hydroxycarbamoyl)- 3-((6-(4-methoxy- phenoxy)pyridin-3- yl)sulfonyl)-3,8- diazabicyclo- [3.2.1]octane-8- carboxylate | ¹H NMR (400 MHZ, DMSO- d₆): δ 10.65 (brs, 1H), 8.77 (brs, 1H), 8.41 (s, 1H), 8.06 (dd, J = 2.4, 8.8 Hz, 1H), 7.20- 7.09 (m, 3H), 7.01-6.97 (m, 2H), 4.50-4.36 (m, 1H), 4.32-4.21 (m, 1H), 4.12- 4.01 (m, 2H), 3.94-3.80 (m, 1H), 3.77 (s, 3H), 3.62-3.38 (m, 4H), 3.23 (s, 3H), 2.02- 1.76 (m, 2H), 1.62-1.47 (m, 2H); MS (ESI): m/z 537.2 [M + H]⁺. |
| 19 | 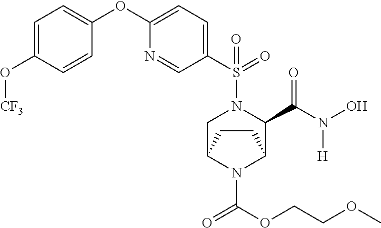 [SMILES: COCCOC(=O)N1[C@@H]2CC[C@H]1[C@H](C(=O)NO)N(S(=O)(=O)c1ccc(Oc3ccc(OC(F)(F)F)cc3)nc1)C2] | 2-methoxyethyl (1S,2R,5R)-2- (hydroxycarbamoyl)- 3-((6-(4-(trifluoro- methoxy)phenoxy)- pyridin-3-yl)- sulfonyl)-3,8-diaza- bicyclo[3.2.1 ]octane- 8-carboxylate | ¹H NMR (400 MHZ, DMSO- d₆): § 10.60 (brs, 1H), 8.77 (brs, 1H), 8.44 (s, 1H), 8.12 (dd, J = 2.4, 8.8 Hz, 1H), 7.46 (d, J = 8.4 Hz, 2H), 7.36 (d, J = 9.2 Hz, 2H), 7.24 (d, J = 8.4 Hz, 1H), 4.70-4.36 (m, 1H), 4.31-4.21 (m, 1H), 4.10- 4.01 (m, 2H), 3.95-3.80 (m, 1H), 3.61-3.35 (m, 4H), 3.21 (s, 3H), 2.01-1.70 (m, 2H), 1.61-1.45 (m, 2H); MS (ESI): m/z 591.1 [M + H]⁺. |
| 20 | 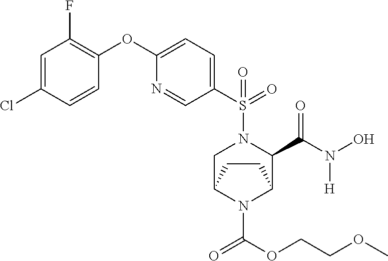 [SMILES: COCCOC(=O)N1[C@@H]2CC[C@H]1[C@H](C(=O)NO)N(S(=O)(=O)c1ccc(Oc3ccc(Cl)cc3F)nc1)C2] | 2-methoxyethyl (1S,2R,5R)-3-((6-(4- chloro-2-fluoro- phenoxy)pyridin-3- yl)sulfonyl)-2- (hydroxycarbamoyl)- 3,8- diazabicyclo[3.2.1]- octane-8-carboxylate | ¹H NMR (400 MHZ, DMSO- d₆): δ 10.66 (brs, 1H), 8.77 (brs, 1H), 8.43 (d, J = 2.4 Hz, 1H), 8.15 (dd, J = 2.4, 8.8 Hz, 1H), 7.67 (dd, J = 2.4, 10.4 Hz, 1H), 7.50-7.35 (m, 3H), 4.50-4.38 (m, 1H), 4.32- 4.22 (m, 1H), 4.12-4.01 (m, 2H), 3.94-3.80 (m, 1H), 3.62- 3.38 (m, 4H), 3.23 (s, 3H), 2.02-1.76 (m, 2H), 1.62- 1.42 (m, 2H); MS (ESI): m/z 559.3 [M + H]⁺. |
| 21 | 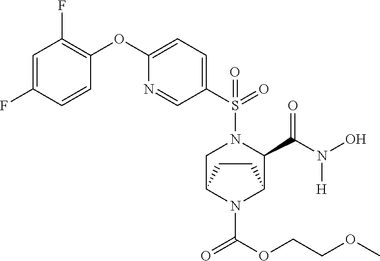 [SMILES: COCCOC(=O)N1[C@@H]2CC[C@H]1[C@H](C(=O)NO)N(S(=O)(=O)c1ccc(Oc3ccc(F)cc3F)nc1)C2] | 2-methoxyethyl (1S,2R,5R)-3-((6- (2,4-difluoro- phenoxy)pyridin-3- yl)sulfonyl)-2- (hydroxycarbamoyl)- 3,8-diazabicyclo- [3.2.1]octane-8- carboxylate | ¹H NMR (400 MHZ, DMSO- d₆): δ 10.65 (brs, 1H), 8.78 (brs, 1H), 8.43 (d, J = 2.4 Hz, 1H), 8.14 (dd, J = 2.4, 8.8 Hz, 1H), 7.52-7.45 (m, 2H), 7.32 (d, J = 8.8 Hz, 1H), 7.21- 7.15 (m, 1H), 4.70-4.40 (m, 1H), 4.30-4.20 (m, 1H), 4.12- 4.01 (m, 2H), 3.96-3.80 (m, 1H), 3.61-3.28 (m, 4H), 3.23 (s, 3H), 2.00-1.70 (m, 2H), 1.69-1.40 (m, 2H); MS (ESI): m/z 541.1 [M-H]-. |
| 22 | 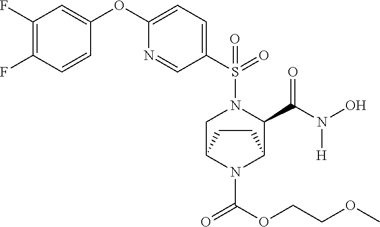 [SMILES: COCCOC(=O)N1[C@@H]2CC[C@H]1[C@H](C(=O)NO)N(S(=O)(=O)c1ccc(Oc3ccc(F)c(F)c3)nc1)C2] | 2-methoxyethyl (1S,2R,5R)-3-((6- (3,4-difluoro- phenoxy)pyridin-3- yl)sulfonyl)-2- (hydroxycarbamoyl)- 3,8-diazabicyclo- [3.2.1]octane-8- carboxylate | ¹H NMR (400 MHZ, DMSO- d₆): δ 10.66 (brs, 1H), 8.77 (brs, 1H), 8.44 (d, J = 2.0 Hz, 1H), 8.11 (dd, J = 2.4, 8.4 Hz, 1H), 7.57-7.46 (m, 2H), 7.23 (d, J = 8.8 Hz, 1H), 7.14- 7.09 (m, 1H), 4.50-4.38 (m, 1H), 4.32-4.22 (m, 1H), 4.12- 3.99 (m, 2H), 3.92-3.84 (m, 1H), 3.62-3.36 (m, 4H), 3.23 (s, 3H), 2.02-1.76 (m, 2H), 1.62-1.48 (m, 2H); MS (ESI): m/z 543.2 [M + H]⁺. |
| 23 | 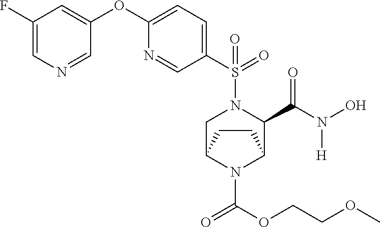 [SMILES: COCCOC(=O)N1[C@@H]2CC[C@H]1[C@H](C(=O)NO)N(S(=O)(=O)c1ccc(Oc3cncc(F)c3)nc1)C2] | 2-methoxyethyl (1S,2R,5R)-3-((6- ((5-fluoropyridin-3- yl)oxy)pyridin-3-yl)- sulfonyl)-2- (hydroxycarbamoyl)- 3,8-diaza- bicyclo[3.2.1 ]octane- 8-carboxylate | ¹H NMR (400 MHZ, DMSO- d₆): δ 10.67 (brs, 1H), 8.77 (brs, 1H), 8.55 (d, J = 2.4 Hz, 1H), 8.46 (d, J = 4.4 Hz, 2H), 8.15 (dd, J = 2.4, 8.8 Hz, 1H), 7.92-7.86 (m, 1H), 7.40 (d, J = 8.8 Hz, 1H), 4.50-4.36 (m, 1H), 4.32-4.22 (m, 1H), 4.12- 4.00 (m, 2H), 3.94-3.84 (m, 1H), 3.62-3.36 (m, 4H), 3.23 (s, 3H), 2.01-1.76 (m, 2H), 1.66-1.48 (m, 2H); MS (ESI): m/z 526.2 [M + H]⁺. |

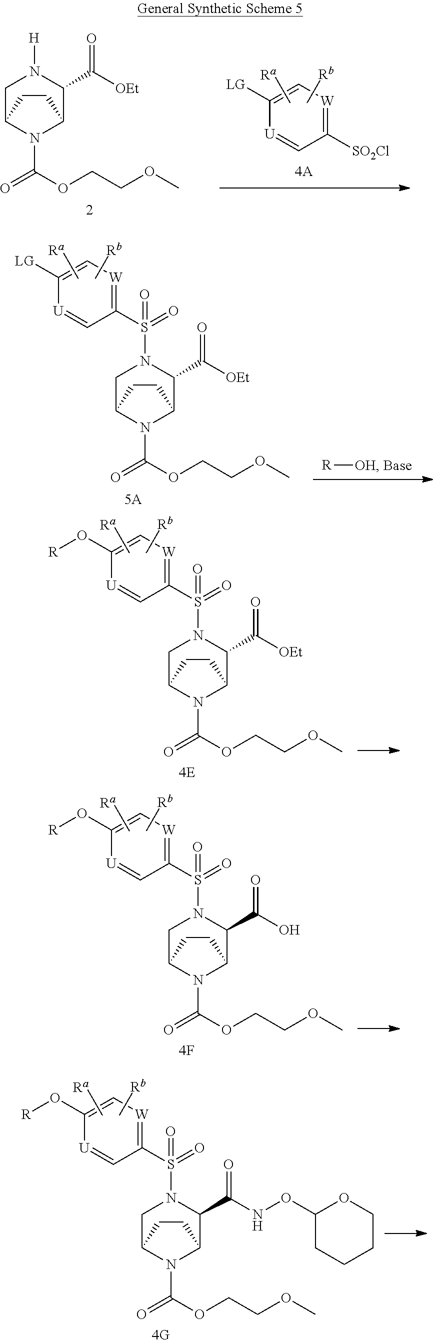

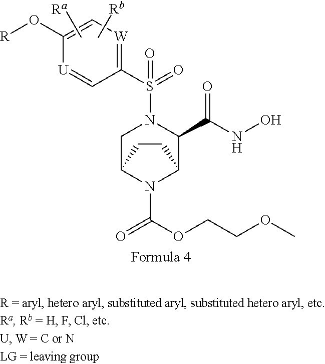

### Example 24

### Synthesis of 2-methoxyethyl (1S,2R,5R)-3-((6-(4-(difluoromethoxy)phenoxy)pyridin-3-yl)sulfonyl)-2-(hydroxycarbamoyl)-3,8-diazabicyclo[3.2.1]octane-8-carboxylate

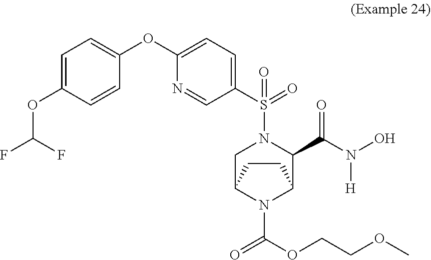 [SMILES: COCCOC(=O)N1[C@@H]2CC[C@H]1[C@H](C(=O)NO)N(S(=O)(=O)c1ccc(Oc3ccc(OC(F)F)cc3)nc1)C2]

### Step 1

### Synthesis of 2-ethyl 8-(2-methoxyethyl) (1S,2S,5R)-3-((6-fluoropyridin-3-yl)sulfonyl)-3,8-diazabicyclo[3.2.1]octane-2,8-dicarboxylate

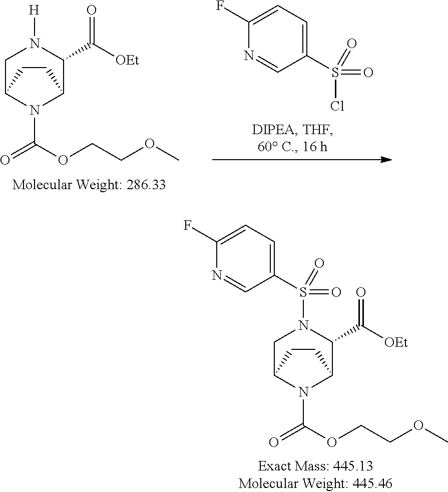 [SMILES: CCOC(=O)[C@H]1NC[C@H]2CC[C@@H]1N2C(=O)OCCOC ; O=S(=O)(Cl)c1ccc(F)nc1 ; CCOC(=O)[C@@H]1[C@@H]2CC[C@H](CN1S(=O)(=O)c1ccc(F)nc1)N2C(=O)OCCOC]

To a stirring solution of 2-ethyl 8-(2-methoxyethyl) (1S,2S,5R)-3,8-diazabicyclo[3.2.1]octane-2,8-dicarboxylate (2 g, 6.98 mmol) in THF (10 mL) under nitrogen at 0-10° C. was added DIPEA (3.7 mL, 20.9 mmol) and stirred for 20 min. 6-Fluoropyridine-3-sulfonyl chloride (2 g, 10.4 mmol) was dissolved in THF (10 mL) and added to the reaction at the same temperature. The reaction was allowed to stir at 60° C. for 16 h while being monitored by TLC. The reaction mixture was cooled to RT, diluted with water (20 mL) and extracted with ethyl acetate (2×30 mL). The combined organic layer was dried over anhydrous Na₂SO₄, filtered and concentrated under reduced pressure to afford crude compound. The crude compound was purified by silica gel column chromatography (100-200 mesh, 15-25% EtOAc in hexanes) to afford 2-ethyl 8-(2-methoxyethyl) (1S,2S,5R)-3-((6-fluoropyridin-3-yl)sulfonyl)-3,8-diazabicyclo[3.2.1]octane-2,8-dicarboxylate (2.1 g, 4.71 mmol, 67% yield) as a light yellow gum. MS (ESI): 446.1 [M+H]⁺; ¹H NMR (400 MHz, CDCl₃): 8.77-8.75 (m, 1H), 8.41-8.38 (m, 1H), 7.11 (dd, J=2.4 Hz, 8.4 Hz, 1H), 4.47-4.39 (m, 2H), 4.38-4.29 (m, 1H), 4.28-4.19 (m, 3H), 4.13-4.06 (m, 1H), 3.58-3.56 (m, 2H), 3.38 (s, 3H), 3.21-3.13 (m, 2H), 2.46-2.38 (m, 1H), 2.00-1.98 (m, 1H), 1.84-1.71 (m, 1H), 1.70-1.62 (m, 1H), 1.34 (t, J=7.2 Hz, 3H).

### Step 2

### Synthesis of 2-ethyl 8-(2-methoxyethyl) (1S,2S,5R)-3-((6-(4-(difluoromethoxy)-phenoxy)pyridin-3-yl)sulfonyl)-3,8-diazabicyclo[3.2.1]octane-2,8-dicarboxylate

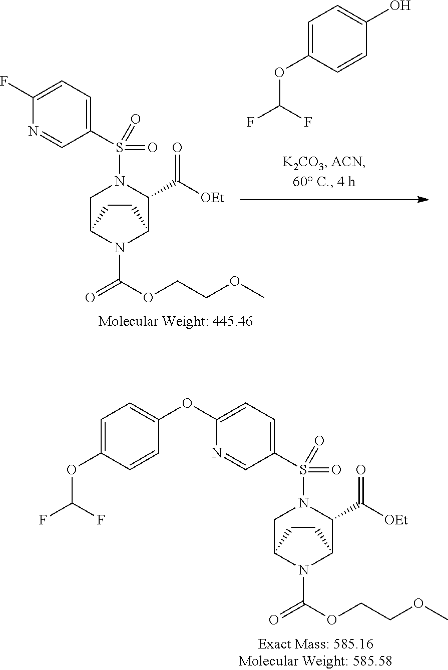 [SMILES: CCOC(=O)[C@@H]1[C@@H]2CC[C@H](CN1S(=O)(=O)c1ccc(F)nc1)N2C(=O)OCCOC ; Oc1ccc(OC(F)F)cc1 ; CCOC(=O)[C@@H]1[C@@H]2CC[C@H](CN1S(=O)(=O)c1ccc(Oc3ccc(OC(F)F)cc3)nc1)N2C(=O)OCCOC]

To a stirring solution of 4-(difluoromethoxy)phenol (204 mg, 1.28 mmol) in ACN (5 mL) under nitrogen at 0-10° C. was added K₂CO₃ (530 mg, 3.84 mmol) and stirred for 20 min. 2-Ethyl 8-(2-methoxyethyl) (1S,2S,5R)-3-((6-fluoropyridin-3-yl)sulfonyl)-3,8-diazabicyclo[3.2.1]octane-2,8-dicarboxylate (570 mg, 1.28 mmol) was dissolved in ACN (10 mL) and added to the reaction at the same temperature. The reaction was stirred at 50° C. for 4 h while being monitored by TLC. The reaction was cooled to room temperature, diluted with water (10 mL) and extracted with ethyl acetate (2×20 mL). The combined organic layer was dried over anhydrous Na₂SO₄, filtered and concentrated under reduced pressure. The crude was purified by silica gel column chromatography (100-200 mesh, 15-30% EtOAc in hexanes) to afford 2-ethyl 8-(2-methoxyethyl) (1S,2S,5R)-3-((6-(4-(difluoromethoxy)phenoxy)pyridin-3-yl)sulfonyl)-3,8-diazabicyclo[3.2.1]octane-2,8-dicarboxylate (450 mg, 0.77 mmol, 60% yield) as a light brown gum. MS (ESI): m/z 586.1 [M+H]⁺.

### Step 3

### Synthesis of (1S,2R,5R)-3-((6-(4-(difluoromethoxy)phenoxy)pyridin-3-yl)sulfonyl)-8-((2-methoxyethoxy)carbonyl)-3,8-diazabicyclo[3.2.1]octane-2-carboxylic acid

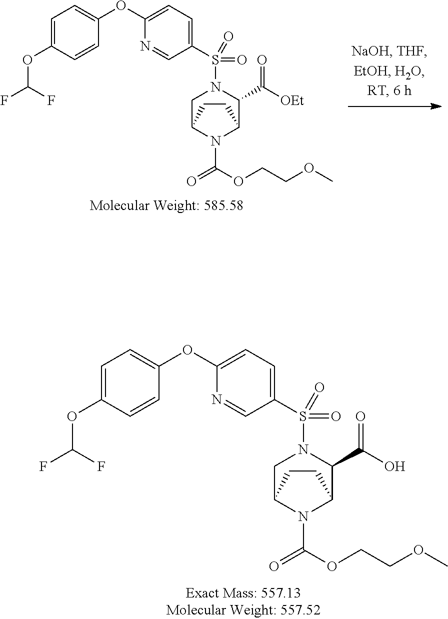 [SMILES: CCOC(=O)[C@@H]1[C@@H]2CC[C@H](CN1S(=O)(=O)c1ccc(Oc3ccc(OC(F)F)cc3)nc1)N2C(=O)OCCOC ; COCCOC(=O)N1[C@@H]2CC[C@H]1[C@H](C(=O)O)N(S(=O)(=O)c1ccc(Oc3ccc(OC(F)F)cc3)nc1)C2]

Similar procedure was followed as described for Step 4 of Example 1, to obtain (1S,2R,5R)-3-((6-(4-(difluoromethoxy)phenoxy)pyridin-3-yl)sulfonyl)-8-((2-methoxyethoxy)carbonyl)-3,8-diazabicyclo[3.2.1]octane-2-carboxylic acid (490 mg, 0.88 mmol, 79% yield) as a light brown gum. This was used as such for the next step without further purification. MS (ESI): m/z 558.1 [M+H]⁺.

### Step 4

### Synthesis of 2-methoxyethyl (1S,2R,5R)-3-((6-(4-(difluoromethoxy)phenoxy)pyridin-3-yl)sulfonyl)-2-(((tetrahydro-2H-pyran-2-yl)oxy)carbamoyl)-3,8-diazabicyclo[3.2.1]octane-8-carboxylate

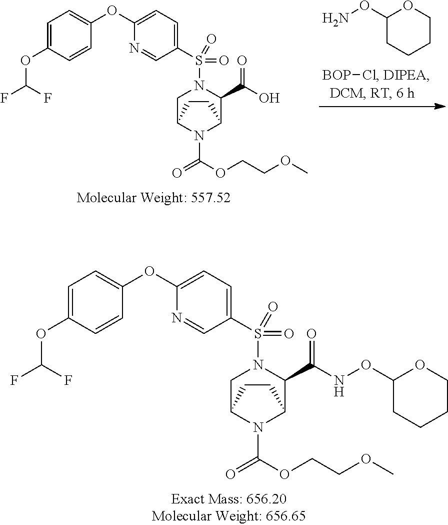

Similar procedure was followed as described for Step 3 of Example 10, to obtain 2-methoxyethyl (1S,2R,5R)-3-((6-(4-(difluoromethoxy)phenoxy)pyridin-3-yl)sulfonyl)-2-(((tetrahydro-2H-pyran-2-yl)oxy)carbamoyl)-3,8-diazabicyclo[3.2.1]octane-8-carboxylate (530 mg, 0.80 mmol) as a brown gum. This was used as such for the next step without further purification. MS (ESI): m/z 657.2 [M+H]⁺.

### Step 5

### Synthesis of 2-methoxyethyl (1S,2R,5R)-3-((6-(4-(difluoromethoxy)phenoxy)pyridin-3-yl)sulfonyl)-2-(hydroxycarbamoyl)-3,8-diazabicyclo[3.2.1]octane-8-carboxylate (Example 24)

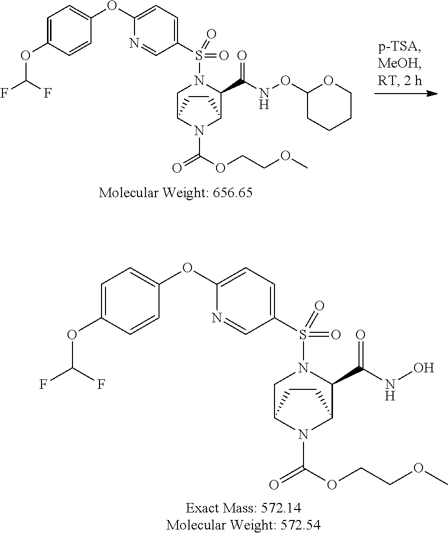 [SMILES: COCCOC(=O)N1[C@@H]2CC[C@H]1[C@H](C(=O)NOC1CCCCO1)N(S(=O)(=O)c1ccc(Oc3ccc(OC(F)F)cc3)nc1)C2 ; COCCOC(=O)N1[C@@H]2CC[C@H]1[C@H](C(=O)NO)N(S(=O)(=O)c1ccc(Oc3ccc(OC(F)F)cc3)nc1)C2]

To a stirred solution of 2-methoxyethyl (1S,2R,5R)-3-((6-(4-(difluoromethoxy)-phenoxy)pyridin-3-yl)sulfonyl)-2-(((tetrahydro-2H-pyran-2-yl)oxy)carbamoyl)-3,8-diazabicyclo[3.2.1]octane-8-carboxylate (530 mg, 0.80 mmol) in MeOH (10 mL) at 0-10° C. was added p-TSA (767 mg, 4.03 mmol). The reaction mixture was stirred at RT for 2 h while being monitored by TLC. The reaction was quenched with aqueous saturated NaHCO₃ solution (10 mL) and extracted with DCM (2×30 mL). The combined organic layer was dried over anhydrous Na₂SO₄, filtered and evaporated under reduced pressure to afford 480 mg of semi pure product which was treated with 20% MTBE in i-PrOAc (4.8 mL) and stirred at room temperature for 16 h. Precipitated solid was filtered, washed with MTBE (2 L), dissolved in DCM (20 mL) and washed with aqueous saturated NaHCO₃ solution (2×5 mL). The organic layer was dried over anhydrous Na₂SO₄, filtered and evaporated under reduced pressure and lyophilized with ACN:H₂O (1:1, 2 mL) to afford 2-methoxyethyl (1S,2R,5R)-3-((6-(4-(difluoromethoxy)phenoxy)pyridin-3-yl)sulfonyl)-2-(hydroxycarbamoyl)-3,8-diazabicyclo[3.2.1]octane-8-carboxylate (Example 24) (130 mg, 0.23 mmol, 26% yield for 2 steps) as an off-white solid. MS (ESI): m/z 573.1 [M+H]⁺; ¹H NMR (400 MHz, DMSO-d₆): δ 10.65 (brs, 1H), 8.77 (brs, 1H), 8.42 (s, 1H), 8.12 (dd, J=2.4, 8.8 Hz, 1H), 7.30-7.26 (m, 4H), 7.25 (t, J=74.0 Hz, 1H), 7.20 (d, J=8.8 Hz, 1H), 4.70-4.35 (m, 1H), 4.32-4.20 (m, 1H), 4.143-3.99 (m, 2H), 3.95-3.80 (m, 1H), 3.64-3.35 (m, 4H), 3.23 (s, 3H), 2.05-1.71 (m, 2H), 1.70-1.48 (m, 2H).

Similarly, below examples were prepared by following the same or analogous process as described in Example 24.

TABLE 7

| Ex. | Structure | Name | Analytical data |
|---|---|---|---|
| 15 | 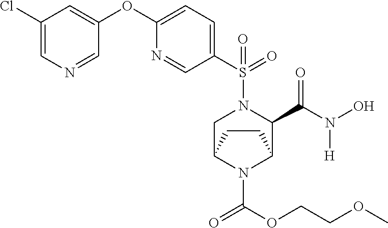 [SMILES: COCCOC(=O)N1[C@@H]2CC[C@H]1[C@H](C(=O)NO)N(S(=O)(=O)c1ccc(Oc3cncc(Cl)c3)nc1)C2] | 2-methoxyethyl (1S,2R,5R)-3-((6- ((5-chloropyridin-3- yl)oxy)pyridin-3-yl)- sulfonyl)-2- (hydroxycarbamoyl)- 3,8-diaza- bicyclo[3.2.1 ]octane- 8-carboxylate | ¹H NMR (400 MHZ, DMSO- d₆): δ 10.67 (brs, 1H), 8.77 (brs, 1H), 8.57 (d, J = 2.0 Hz, 1H), 8.54 (d, J = 2.4 Hz, 1H), 8.45 (s, 1H), 8.15 (dd, J = 2.4, 8.8 Hz, 1H), 8.07-8.05 (m, 1H), 7.34 (d, J = 8.8 Hz, 1H), 4.67-4.37 (m, 1H), 4.33- 4.21 (m, 1H), 4.12-3.99 (m, 2H), 3.98-3.80 (m, 1H), 3.61- 3.55 (m, 1H), 3.54-3.39 (m, 3H), 3.26 (s, 3H), 2.02- 1.75 (m, 2H), 1.69-1.40 (m, 2H); MS (ESI): m/z 542.1 [M + H]⁺. |
| 16 | 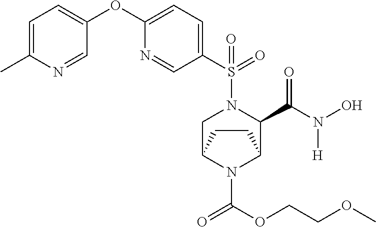 | 2-methoxyethyl (1S,2R,5R)-2- (hydroxycarbamoyl)- 3-((6-((6-methyl- pyridin-3-yl)oxy)- pyridin-3-yl)- sulfonyl)-3,8-diaza- bicyclo[3.2.1 ]octane- 8-carboxylate | ¹H NMR (400 MHZ, DMSO- d₆): δ 10.66 (brs, 1H), 8.77 (brs, 1H), 8.41 (s, 1H), 8.37 (d, J = 2.8 Hz, 1H), 8.11 (dd, J = 2.4, 8.8 Hz, 1H), 7.61 - 7.57 (m, 1H), 7.35 (d, J = 8.4 Hz, 1H), 7.26 (d, J = 8.8 Hz, 1H), 4.62-4.37 (m, 1H), 4.32- 4.21 (m, 1H), 4.13-3.99 (m, 2H), 3.95-3.80 (m, 1H), 3.62-3.35 (m, 4H), 3.23 (s, 3H), 2.23-1.73 (m, 3H), 1.70- 1.40 (m, 4H); MS (ESI): m/z 522.2 [M + H]⁺. |
| 25 | 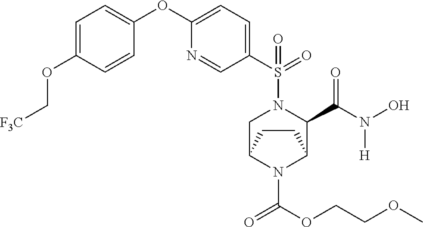 [SMILES: COCCOC(=O)N1[C@@H]2CC[C@H]1[C@H](C(=O)NO)N(S(=O)(=O)c1ccc(Oc3ccc(OCC(F)(F)F)cc3)nc1)C2] | 2-methoxyethyl (1S,2R,5R)-2- (hydroxycarbamoyl)- 3-((6-(4-(2,2,2- trifluoroethoxy)- phenoxy)pyridin-3- yl)sulfonyl)-3,8- diazabicyclo[3.2.1]- octane-8-carboxylate | ¹H NMR (400 MHZ, DMSO- d₆): δ 10.65 (brs, 1H), 8.77 (brs, 1H), 8.42 (d, J = 2.4 Hz, 1H), 8.08 (dd, J = 2.4, 8.8 Hz, 1H), 7.20-7.11 (m, 5H), 4.79 (q, J = 8.8 Hz, 2H), 4.62- 4.36 (m, 1H), 4.30-4.20 (m, 1H), 4.10-4.01 (m, 2H), 3.95- 3.80 (m, 1H), 3.61-3.34 (m, 4H), 3.23 (s, 3H), 2.00- 1.72 (m, 2H), 1.65-1.45 (m, 2H); MS (ESI): m/z 605.2 [M + H]⁺. |
| 26 | 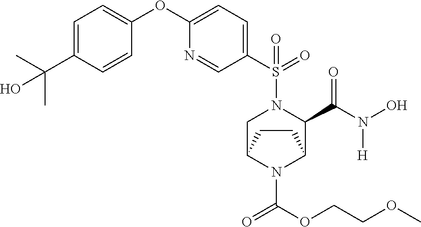 [SMILES: COCCOC(=O)N1[C@@H]2CC[C@H]1[C@H](C(=O)NO)N(S(=O)(=O)c1ccc(Oc3ccc(C(C)(C)O)cc3)nc1)C2] | 2-methoxyethyl (1S,2R,5R)-2- (hydroxycarbamoyl)- 3-((6-(4-(2-hydroxy- propan-2-yl)- phenoxy)pyridin-3- yl)sulfonyl)-3,8- diazabicyclo[3.2.1]- octane-8-carboxylate | ¹H NMR (400 MHZ, DMSO- d₆): δ 10.66 (brs, 1H), 8.79 (brs, 1H), 8.42 (s, 1H), 8.08 (dd, J = 2.0, 8.6 Hz, 1H), 7.52 (d, J = 8.4 Hz, 2H), 7.16- 7.08 (m, 3H), 5.07 (brs, 1H), 4.65-4.37 (m, 1H), 4.31- 4.20 (m, 1H), 4.12-4.00 (m, 2H), 3.95-3.80 (m, 1H), 3.61- 3.34 (m, 4H), 3.23 (s, 3H), 2.00-1.70 (m, 2H), 1.69- 1.48 (m, 2H), 1.45 (s, 6H); MS (ESI): m/z 565.4 [M + H]⁺. |
| 27 | 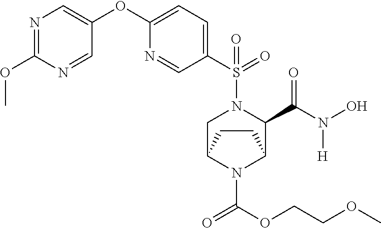 [SMILES: COCCOC(=O)N1[C@@H]2CC[C@H]1[C@H](C(=O)NO)N(S(=O)(=O)c1ccc(Oc3cnc(OC)nc3)nc1)C2] | 2-methoxyethyl (1S,2R,5R)-2- (hydroxycarbamoyl)- 3-((6-((2-methoxy- pyrimidin-5-yl)oxy)- pyridin-3-yl)- sulfonyl)-3,8-diaza- bicyclo[3.2.1]octane- 8-carboxylate | ¹H NMR (400 MHZ, DMSO- d₆): δ 10.67 (brs, 1H), 8.81 (brs, 1H), 8.71 (s, 2H), 8.43 (s, 1H), 8.37 (dd, J = 2.4, 8.8 Hz, 1H), 7.34 (d, J = 8.8 Hz, 1H), 4.62-4.35 (m, 1H), 4.32- 4.20 (m, 1H), 4.13-3.99 (m, 2H), 3.95 (s, 3H), 3.93- 3.78 (m, 1H), 3.62-3.35 (m, 4H), 3.23 (s, 3H), 2.20-1.75 (m, 2H), 1.68-1.48 (m, 2H); MS (ESI): m/z 539.3 [M + H]⁺. |
| 28 | 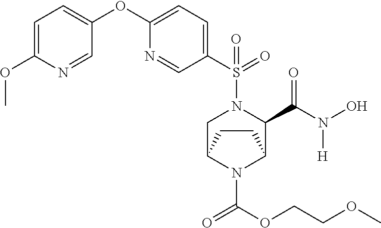 [SMILES: COCCOC(=O)N1[C@@H]2CC[C@H]1[C@H](C(=O)NO)N(S(=O)(=O)c1ccc(Oc3ccc(OC)nc3)nc1)C2] | 2-methoxyethyl (1S,2R,5R)-2- (hydroxycarbamoyl)- 3-((6-((6-methoxy- pyridin-3-yl)oxy)- pyridin-3-yl)- sulfonyl)-3,8-diaza- bicyclo[3.2.1]octane- 8-carboxylate | ¹H NMR (400 MHZ, DMSO- d₆): δ 10.66 (brs, 1H), 8.77 (brs, 1H), 8.42 (d, J = 2.0 Hz, 1H), 8.13-8.06 (m, 2H), 7.67- 7.62 (m, 1H), 7.24 (d, J = 8.8 Hz, 1H), 6.91 (d, J = 8.8 Hz, 1H), 4.70-4.39 (m, 1H), 4.30-4.20 (m, 1H), 4.13- 4.00 (m, 2H), 3.87 (s, 3H), 3.85-3.70 (m, 1H), 3.62- 3.35 (m, 4H), 3.23 (s, 3H), 2.00-1.75 (m, 2H), 1.65- 1.35 (m, 2H); MS (ESI): m/z 538.2 [M + H]⁺. |
| 29 | 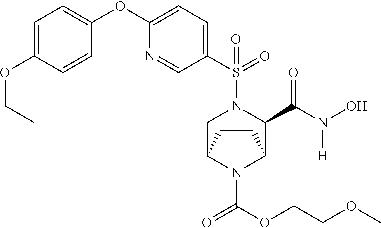 [SMILES: CCOc1ccc(Oc2ccc(S(=O)(=O)N3C[C@H]4CC[C@@H]([C@@H]3C(=O)NO)N4C(=O)OCCOC)cn2)cc1] | 2-methoxyethyl (1S,2R,5R)-3-((6-(4- ethoxyphenoxy)- pyridin-3-yl)- sulfonyl)-2- (hydroxycarbamoyl)- 3,8-diazabicyclo- [3.2.1]octane-8- carboxylate | ¹H NMR (400 MHZ, DMSO- d₆): δ 10.68 (brs, 1H), 8.79 (brs, 1H), 8.42 (d, J = 2.4 Hz, 1H), 8.06 (dd, J = 2.4, 8.8 Hz, 1H), 7.13-7.09 (m, 3H), 7.00- 6.96 (m, 2H), 4.70-4.35 (m, 1H), 4.30-4.20 (m, 1H), 4.15-3.98 (m, 4H), 3.92- 3.80 (m, 1H), 3.61-3.35 (m, 4H), 3.23 (s, 3H), 2.02-1.74 (m, 2H), 1.68-1.40 (m, 2H), 1.34 (t, J = 7.2 Hz, 3H); MS (ESI): m/z 551.2 [M + H]⁺. |
| 30 | 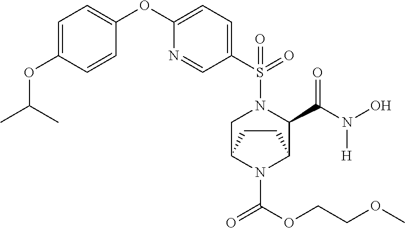 [SMILES: COCCOC(=O)N1[C@@H]2CC[C@H]1[C@H](C(=O)NO)N(S(=O)(=O)c1ccc(Oc3ccc(OC(C)C)cc3)nc1)C2] | 2-methoxyethyl (1S,2R,5R)-2- (hydroxycarbamoyl)- 3-((6-(4-isopropoxy- phenoxy)pyridin-3- yl)sulfonyl)-3,8- diazabicyclo[3.2.1]- octane-8-carboxylate | ¹H NMR (400 MHZ, DMSO- d₆): δ 10.65 (brs, 1H), 8.77 (brs, 1H), 8.42 (s, 1H), 8.06 (dd, J = 2.4, 8.8 Hz, 1H), 7.12- 7.09 (m, 3H), 6.97-6.95 (m, 2H), 4.62-4.56 (m, 1H), 4.55-4.35 (m, 1H), 4.30- 4.20 (m, 1H), 4.13-3.98 (m, 2H), 3.95-3.80 (m, 1H), 3.61- 3.35 (m, 4H), 3.23 (s, 3H), 2.02-1.74 (m, 2H), 1.65- 1.45 (m, 2H), 1.26 (d, J = 6.0 Hz, 6H); MS (ESI): m/z 563.3 [M + H]⁺. |
| 31 | 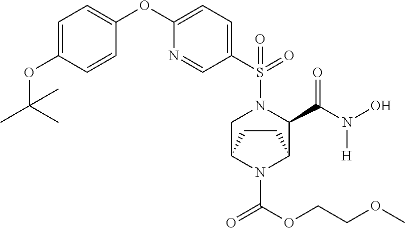 [SMILES: COCCOC(=O)N1[C@@H]2CC[C@H]1[C@H](C(=O)NO)N(S(=O)(=O)c1ccc(Oc3ccc(OC(C)(C)C)cc3)nc1)C2] | 2-methoxyethyl (1S,2R,5R)-3-((6-(4- (tert-butoxy)- phenoxy)pyridin-3- yl)sulfonyl)-2- (hydroxycarbamoyl)- 3,8-diazabicyclo- [3.2.1]octane-8- carboxylate | ¹H NMR (400 MHZ, DMSO- d₆): δ 10.66 (brs, 1H), 8.78 (brs, 1H), 8.45 (d, J = 2.0 Hz, 1H), 8.08 (dd, J = 2.8, 8.8 Hz, 1H), 7.14-7.09 (m, 3H), 7.05- 7.01 (m, 2H), 4.70-4.35 (m, 1H), 4.31-4.20 (m, 1H), 4.13-3.99 (m, 2H), 3.96- 3.75 (m, 1H), 3.62-3.35 (m, 4H), 3.23 (s, 3H), 2.05-1.75 (m, 2H), 1.70-1.45 (m, 2H), 1.32 (s, 9H); MS (ESI): m/z 579.2 [M + H]⁺. |
| 32 | 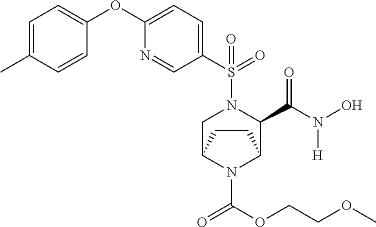 | 2-methoxyethyl (1S,2R,5R)-2- (hydroxycarbamoyl)- 3-((6-(p-tolyloxy)- pyridin-3-yl)- sulfonyl)-3,8- diazabicyclo[3.2.1]- octane-8-carboxylate | ¹H NMR (400 MHZ, DMSO- d₆): δ 10.68 (brs, 1H), 8.80 (brs, 1H), 8.41 (d, J = 2.0 Hz, 1H), 8.07 (dd, J = 2.4, 8.6 Hz, 1H), 7.23 (d, J = 8.0 Hz, 2H), 7.14-7.05 (m, 3H), 4.65- 4.35 (m, 1H), 4.31-4.20 (m, 1H), 4.13-3.99 (m, 2H), 3.96- 3.75 (m, 1H), 3.62-3.35 (m, 4H), 3.23 (s, 3H), 2.33 (s, 3H), 2.00-1.70 (m, 2H), 1.68- 1.46 (m, 2H); MS (ESI): m/z 521.2 [M + H]⁺. |
| 33 | 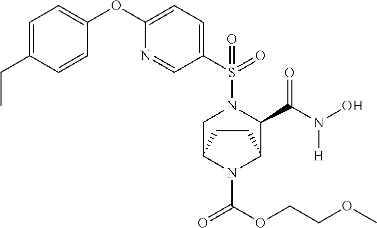 [SMILES: CCc1ccc(Oc2ccc(S(=O)(=O)N3C[C@H]4CC[C@@H]([C@@H]3C(=O)NO)N4C(=O)OCCOC)cn2)cc1] | 2-methoxyethyl (1S,2R,5R)-3-((6-(4- ethylphenoxy)pyridi n-3-yl)sulfonyl)-2- (hydroxycarbamoyl)- 3,8-diazabicyclo- [3.2.1]octane-8- carboxylate | ¹H NMR (400 MHZ, DMSO- d₆): δ 10.71 (brs, 1H), 8.80 (brs, 1H), 8.42 (s, 1H), 8.08 (dd, J = 2.4, 8.2 Hz, 1H), 7.29 (d, J = 8.4 Hz, 2H), 7.15- 7.08 (m, 3H), 4.65-4.35 (m, 1H), 4.31-4.20 (m, 1H), 4.12- 4.01 (m, 2H), 3.95-3.80 (m, 1H), 3.61-3.35 (m, 4H), 3.24 (s, 3H), 2.64 (q, J = 7.6 Hz, 2H), 2.01-1.76 (m, 2H), 1.68-1.46 (m, 2H), 1.21 (t, J = 7.6 Hz, 3H); MS (ESI): m/z 535.3 [M + H]⁺. |
| 34 | 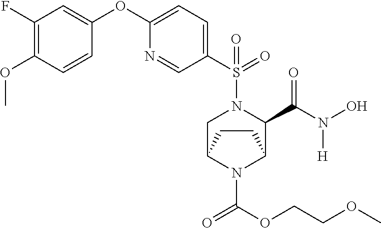 [SMILES: COCCOC(=O)N1[C@@H]2CC[C@H]1[C@H](C(=O)NO)N(S(=O)(=O)c1ccc(Oc3ccc(OC)c(F)c3)nc1)C2] | 2-methoxyethyl (1S,2R,5R)-3-((6-(3- fluoro-4-methoxy- phenoxy)pyridin-3- yl)sulfonyl)-2- (hydroxycarbamoyl)- 3,8-diaza- bicyclo[3.2.1 ]octane- 8-carboxylate | ¹H NMR (400 MHZ, DMSO- d₆): δ 10.71 (brs, 1H), 8.78 (brs, 1H), 8.43 (d, J = 2.0 Hz, 1H), 8.08 (dd, J = 2.0, 8.6 Hz, 1H), 7.26-7.15 (m, 3H), 7.04- 7.01 (m, 1H), 4.70-4.32 (m, 1H), 4.31-4.20 (m, 1H), 4.13-3.98 (m, 2H), 3.86 (s, 3H), 3.86-3.75 (m, 1H), 3.61- 3.35 (m, 4H), 3.23 (s, 3H), 2.03-1.72 (m, 2H), 1.70- 1.45 (m, 2H); MS (ESI): m/z 555.2 [M + H]⁺. |
| 35 | 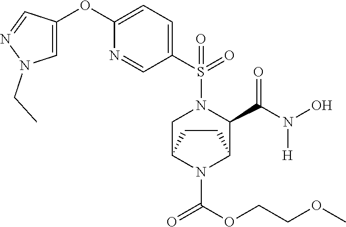 [SMILES: CCn1cc(Oc2ccc(S(=O)(=O)N3C[C@H]4CC[C@@H]([C@@H]3C(=O)NO)N4C(=O)OCCOC)cn2)cn1] | 2-methoxyethyl (1S,2R,5R)-3-((6- ((1-ethyl-1H- pyrazol-4-yl)- oxy)pyridin-3-yl)- sulfonyl)-2- (hydroxycarbamoyl)- 3,8-diaza- bicyclo[3.2.1 ]octane- 8-carboxylate | ¹H NMR (400 MHZ, DMSO- d₆): δ 10.68 (brs, 1H), 8.80 (brs, 1H), 8.48 (s, 1H), 8.06 (d, J = 8.8 Hz, 1H), 7.96 (s, 1H), 7.50 (s, 1H), 7.16 (d, J = 8.8 Hz, 1H), 4.65-4.35 (m, 1H), 4.32-4.20 (m, 1H), 4.11 (q, J = 7.6 Hz, 2H), 4.10- 3.99 (m, 2H), 3.98-3.80 (m, 1H), 3.61-3.35 (m, 4H), 3.22 (s, 3H), 2.01-1.72 (m, 2H), 1.70-1.40 (m, 2H), 1.38 (t, J = 7.6 Hz, 3H); MS (ESI): m/z 525.2 [M + H]⁺. |
| 36 | 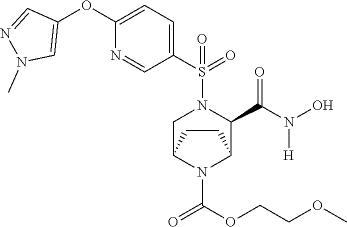 [SMILES: COCCOC(=O)N1[C@@H]2CC[C@H]1[C@H](C(=O)NO)N(S(=O)(=O)c1ccc(Oc3cnn(C)c3)nc1)C2] | 2-methoxyethyl (1S,2R,5R)-2- (hydroxycarbamoyl)- 3-((6-((1-methyl-1H- pyrazol-4-yl)oxy)- pyridin-3-yl)- sulfonyl)-3,8- diazabicyclo[3.2.1]- octane-8-carboxylate | ¹H NMR (400 MHZ, DMSO- d₆): δ 10.65 (brs, 1H), 8.80 (brs, 1H), 8.47 (s, 1H), 8.06 (d, J = 8.8 Hz, 1H), 7.91 (s, 1H), 7.49 (s, 1H), 7.17 (d, J = 8.8 Hz, 1H), 4.62-4.35 (m, 1H), 4.34-4.20 (m, 1H), 4.12- 3.98 (m, 2H), 3.83 (s, 3H), 3.83-3.70 (m, 1H), 3.61- 3.35 (m, 4H), 3.22 (s, 3H), 2.02-1.71 (m, 2H), 1.70- 1.40 (m, 2H); MS (ESI): m/z 511.3 [M + H]⁺. |
| 37 | 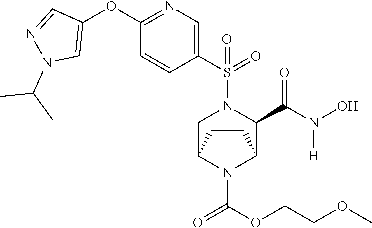 [SMILES: COCCOC(=O)N1[C@@H]2CC[C@H]1[C@H](C(=O)NO)N(S(=O)(=O)c1ccc(Oc3cnn(C(C)C)c3)nc1)C2] | 2-methoxyethyl (1S,2R,5R)-2- (hydroxycarbamoyl)- 3-((6-((1-isopropyl- 1H-pyrazol-4-yl)- oxy)pyridin-3-yl)- sulfonyl)-3,8- diazabicyclo- [3.2.1]octane-8- carboxylate | ¹H NMR (400 MHZ, DMSO- d₆): δ 10.66 (brs, 1H), 8.78 (brs, 1H), 8.48 (s, 1H), 8.06 (dd, J = 1.6, 8.6 Hz, 1H), 7.97 (s, 1H), 7.50 (s, 1H), 7.15 (d, J = 8.4 Hz, 1H), 4.70-4.35 (m, 2H), 4.30-4.20 (m, 1H), 4.15-3.99 (m, 2H), 3.95- 3.79 (m, 1H), 3.65-3.35 (m, 4H), 3.23 (s, 3H), 2.00-1.72 (m, 2H), 1.68-1.46 (m, 2H), 1.42 (d, J = 6.4 Hz, 6H); MS (ESI): m/z 539.2 [M + H]⁺. |
| 38 | 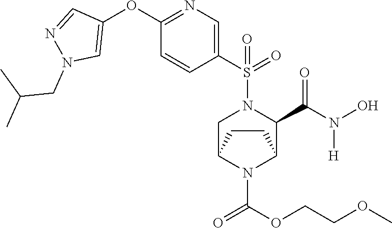 [SMILES: COCCOC(=O)N1[C@@H]2CC[C@H]1[C@H](C(=O)NO)N(S(=O)(=O)c1ccc(Oc3cnn(CC(C)C)c3)nc1)C2] | 2-methoxyethyl (1S,2R,5R)-2- (hydroxycarbamoyl)- 3-((6-((1-isobutyl- 1H-pyrazol-4- yl)oxy)pyridin-3- yl)sulfonyl)-3,8- diazabicyclo- [3.2.1 ]octane-8- carboxylate | ¹H NMR (400 MHz, DMSO- d₆): δ 10.66 (brs, 1H), 8.77 (brs, 1H), 8.48 (d, J = 2.4 Hz, 1H), 8.07 (dd, J = 2.4, 8.8 Hz, 1H), 7.93 (s, 1H), 7.51 (s, 1H), 7.15 (d, J = 8.8 Hz, 1H), 4.70-4.35 (m, 1H), 4.32- 4.20 (m, 1H), 4.15-3.99 (m, 3H), 3.88 (d, J = 7.2 Hz, 2H), 3.64-3.35 (m, 4H), 3.23 (s, 3H), 2.16-2.09 (m, 1H), 2.03- 1.72 (m, 2H), 1.70-1.46 (m, 2H), 0.85 (d, J = 6.8 Hz, 6H); MS (ESI): m/z 553.3 [M + H]⁺. |
| 39 | 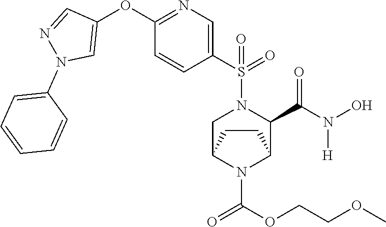 [SMILES: COCCOC(=O)N1[C@@H]2CC[C@H]1[C@H](C(=O)NO)N(S(=O)(=O)c1ccc(Oc3cnn(-c4ccccc4)c3)nc1)C2] | 2-methoxyethyl (1S,2R,5R)-2- (hydroxycarbamoyl)- 3-((6-((1-phenyl-1H- pyrazol-4-yl)oxy)- pyridin-3- yl)sulfonyl)-3,8- diazabicyclo- [3.2.1]octane-8- carboxylate | ¹H NMR (400 MHZ, DMSO- d₆): δ 10.70 (brs, 1H), 8.78 (brs, 1H), 8.74 (d, J = 0.8 Hz, 1H), 8.52 (d, J = 2.4 Hz, 1H), 8.11 (dd, J = 2.4, 8.8 Hz, 1H), 7.86-7.82 (m, 3H), 7.54- 7.49 (m, 2H), 7.35-7.30 (m, 1H), 7.27 (d, J = 8.8 Hz, 1H), 4.69-4.36 (m, 1H), 4.30- 4.21 (m, 1H), 4.18-3.97 (m, 2H), 3.65-3.54 (m, 1H), 3.75- 3.69 (m, 1H), 3.62-3.39 (m, 3H), 3.23 (s, 3H), 2.01- 1.74 (m, 2H), 1.69-1.46 (m, 2H); MS (ESI): m/z 573.3 [M + H]⁺. |
| 40 | 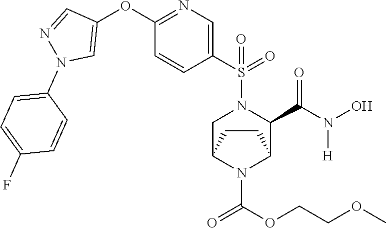 [SMILES: COCCOC(=O)N1[C@@H]2CC[C@H]1[C@H](C(=O)NO)N(S(=O)(=O)c1ccc(Oc3cnn(-c4ccc(F)cc4)c3)nc1)C2] | 2-methoxyethyl (1S,2R,5R)-3-((6- ((1-(4-fluorophenyl)- 1H-pyrazol-4- yl)oxy)pyridin-3- yl)sulfonyl)-2- (hydroxycarbamoyl)- 3,8-diazabicyclo- [3.2.1 ]octane-8- carboxylate | ¹H NMR (400 MHz, DMSO- d₆): δ 10.68 (brs, 1H), 8.77 (brs, 1H), 8.72 (s, 1H), 8.51 (d, J = 2.4 Hz, 1H), 8.11 (dd, J = 2.4, 8.8 Hz, 1H), 7.87- 7.84 (m, 3H), 7.40-7.33 (m, 2H), 7.26 (d, J = 8.8 Hz, 1H), 4.70-4.35 (m, 1H), 4.32- 4.20 (m, 1H), 4.11-3.98 (m, 2H), 3.90-3.80 (m, 1H), 3.62- 3.37 (m, 4H), 3.23 (s, 3H), 2.01-1.74 (m, 2H), 1.69- 1.44 (m, 2H); MS (ESI): m/z 591.2 [M + H]⁺ |

### Example 41

### Synthesis of 2-methoxyethyl (1S,2R,5R)-3-((3-fluoro-4-((1-methyl-1H-pyrazol-4-yl)oxy)phenyl)sulfonyl)-2-(hydroxycarbamoyl)-3,8-diazabicyclo[3.2.1]octane-8-carboxylate

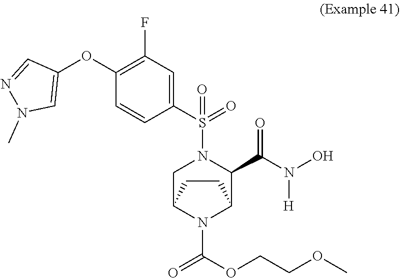 [SMILES: COCCOC(=O)N1[C@@H]2CC[C@H]1[C@H](C(=O)NO)N(S(=O)(=O)c1ccc(Oc3cnn(C)c3)c(F)c1)C2]

### Step 1

### Synthesis of 2-ethyl 8-(2-methoxyethyl) (1S,2S,5R)-3-((3,4-difluorophenyl)sulfonyl)-3,8-diazabicyclo[3.2.1]octane-2,8-dicarboxylate

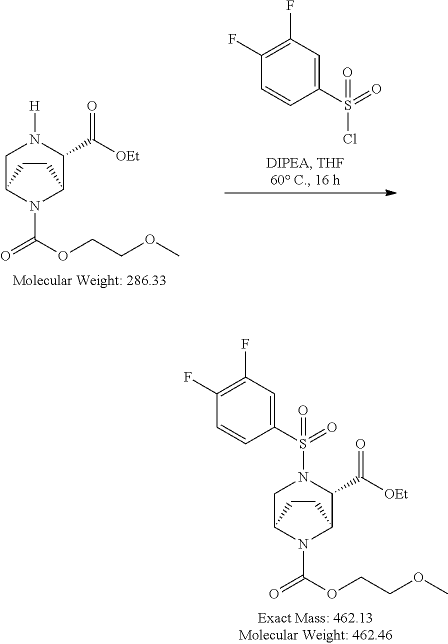 [SMILES: CCOC(=O)[C@H]1NC[C@H]2CC[C@@H]1N2C(=O)OCCOC ; O=S(=O)(Cl)c1ccc(F)c(F)c1 ; CCOC(=O)[C@@H]1[C@@H]2CC[C@H](CN1S(=O)(=O)c1ccc(F)c(F)c1)N2C(=O)OCCOC]

To a stirring solution of 2-ethyl 8-(2-methoxyethyl) (1S,2S,5R)-3,8-diazabicyclo[3.2.1]octane-2,8-dicarboxylate (500 mg, 1.74 mmol) in THF (5 mL) under nitrogen at 0-10° C. was added DIPEA (1.2 mL, 5.2 mmol) and stirred for 20 min. 3,4-Difluorobenzenesulfonyl chloride (557 mg, 2.61 mmol) was dissolved in THF (5 mL) and added to the reaction at the same temperature. The reaction was allowed to stir at 60° C. for 16 h while being monitored by TLC. The reaction mixture was cooled to RT, diluted with water (10 mL) and extracted with ethyl acetate (2×20 mL). The combined organic layer was dried over anhydrous Na₂SO₄, filtered and concentrated under reduced pressure to afford crude product. The crude product was purified by silica gel column chromatography (60-120 mesh, 15-25% EtOAc in hexanes) to afford 2-ethyl 8-(2-methoxyethyl) (1S,2S,5R)-3-((3,4-difluorophenyl)sulfonyl)-3,8-diazabicyclo[3.2.1]octane-2,8-dicarboxylate (360 mg, 0.78 mmol, 44% yield) as a colorless gum. MS (ESI): 463.2 [M+H]⁺.

### Step 2

### Synthesis of 2-ethyl 8-(2-methoxyethyl) (1S,2S,5R)-3-((3-fluoro-4-((1-methyl-1H-pyrazol-4-yl)oxy)phenyl)sulfonyl)-3,8-diazabicyclo[3.2.1]octane-2,8-dicarboxylate

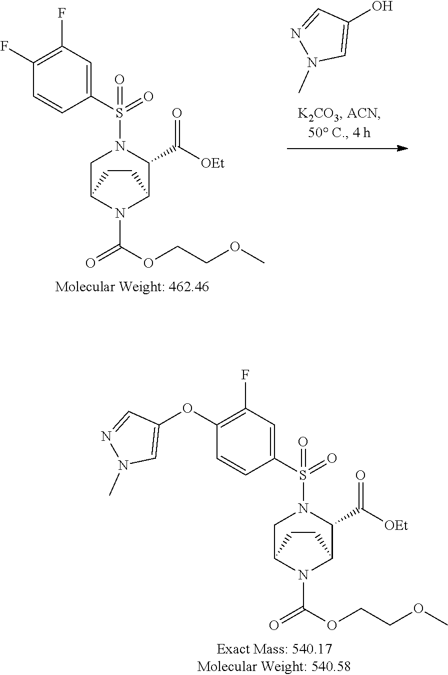

To a stirring solution of 1-methyl-1H-pyrazol-4-ol (115 mg, 1.17 mmol) in ACN (4 mL) under nitrogen at 0-10° C. was added K₂CO₃ (322 mg, 2.34 mmol) and stirred for 20 min. 2-Ethyl 8-(2-methoxyethyl) (1S,2S,5R)-3-((3,4-difluorophenyl)sulfonyl)-3,8-diazabicyclo[3.2.1]octane-2,8-dicarboxylate (360 mg, 0.78 mmol) was dissolved in ACN (6 mL) and added to the reaction at the same temperature. The reaction was stirred at 50° C. for 6 h while being monitored by TLC. The reaction was cooled to room temperature, diluted with water (10 mL) and extracted with ethyl acetate (2×20 mL). The combined organic layer was dried over anhydrous Na₂SO₄, filtered and concentrated under reduced pressure. The crude was purified by silica gel column chromatography (100-200 mesh, 10-25% EtOAc in hexanes) to afford 2-ethyl 8-(2-methoxyethyl) (1S,2S,5R)-3-((3-fluoro-4-((1-methyl-1H-pyrazol-4-yl)oxy)phenyl)sulfonyl)-3,8-diazabicyclo[3.2.1]octane-2,8-dicarboxylate (300 mg, 0.55 mmol, 71% yield) as a colorless liquid. MS (ESI): m/z 541.2 [M+H]⁺.

### Step 3

### Synthesis of (1S,2R,5R)-3-((3-fluoro-4-((1-methyl-1H-pyrazol-4-yl)oxy)phenyl)sulfonyl)-8-((2-methoxyethoxy)carbonyl)-3,8-diazabicyclo[3.2.1]octane-2-carboxylic acid

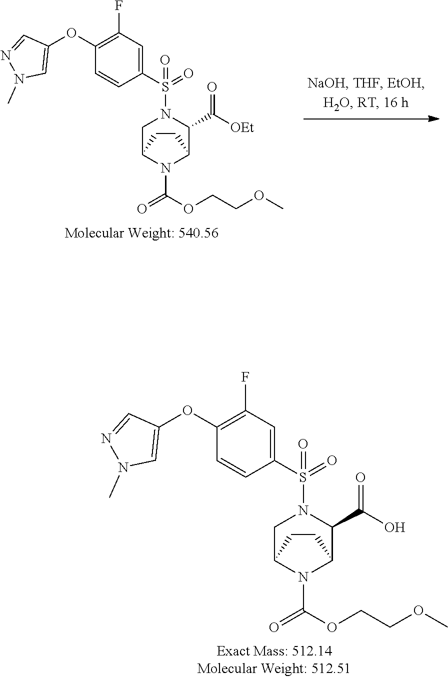 [SMILES: CCOC(=O)[C@@H]1[C@@H]2CC[C@H](CN1S(=O)(=O)c1ccc(Oc3cnn(C)c3)c(F)c1)N2C(=O)OCCOC ; COCCOC(=O)N1[C@@H]2CC[C@H]1[C@H](C(=O)O)N(S(=O)(=O)c1ccc(Oc3cnn(C)c3)c(F)c1)C2]

Similar procedure was followed as described for Step 4 of Example 1, to obtain (1S,2R,5R)-3-((3-fluoro-4-((1-methyl-1H-pyrazol-4-yl)oxy)phenyl)sulfonyl)-8-((2-methoxyethoxy)carbonyl)-3,8-diazabicyclo[3.2.1]octane-2-carboxylic acid (220 mg, 0.43 mmol, 80% yield) as a colorless gum. This was used as such for the next step without further purification. MS (ESI): m/z 513.2 [M+H]⁺.

### Step 4

### Synthesis of 2-methoxyethyl (1S,2R,5R)-3-((3-fluoro-4-((1-methyl-1H-pyrazol-4-yl)-oxy)phenyl)sulfonyl)-2-(((tetrahydro-2H-pyran-2-yl)oxy)carbamoyl)-3,8-diazabicyclo[3.2.1]octane-8-carboxylate

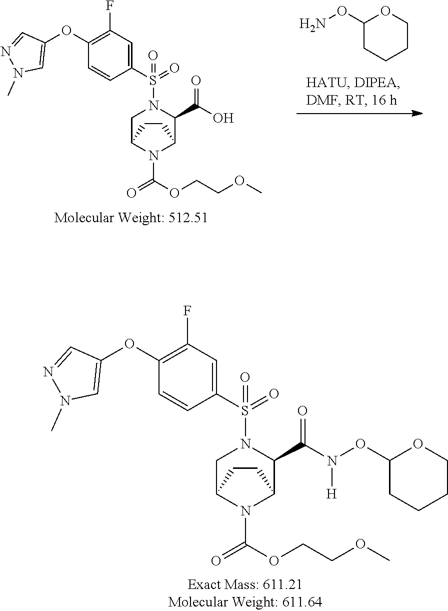 [SMILES: COCCOC(=O)N1[C@@H]2CC[C@H]1[C@H](C(=O)O)N(S(=O)(=O)c1ccc(Oc3cnn(C)c3)c(F)c1)C2 ; NOC1CCCCO1 ; COCCOC(=O)N1[C@@H]2CC[C@H]1[C@H](C(=O)NOC1CCCCO1)N(S(=O)(=O)c1ccc(Oc3cnn(C)c3)c(F)c1)C2]

To a stirred solution of (1S,2R,5R)-3-((3-fluoro-4-((1-methyl-1H-pyrazol-4-yl)oxy)phenyl)sulfonyl)-8-((2-methoxyethoxy)carbonyl)-3,8-diazabicyclo[3.2.1]octane-2-carboxylic acid (210 mg, 0.41 mmol) in DMF (4 mL) at 0-10° C. was added HATU (233 mg, 0.61 mmol) and DIPEA (0.22 mL, 1.23 mmol), and stirred for 30 min. O-(tetrahydro-2H-pyran-2-yl)hydroxylamine (62 mg, 0.53 mmol) was added, warm to RT over a period of 30 min and stirred for 16 h while being monitored by TLC. The reaction was quenched with cold water (10 mL) and the aqueous layer was extracted with EtOAc (2×20 mL). The combined organic layer was washed with brine (15 mL), dried over anhydrous Na₂SO₄, filtered and concentrated under reduced pressure to afford 2-methoxyethyl (1S,2R,5R)-3-((3-fluoro-4-((1-methyl-1H-pyrazol-4-yl)oxy)phenyl)sulfonyl)-2-(((tetrahydro-2H-pyran-2-yl)oxy)carbamoyl)-3,8-diazabicyclo-[3.2.1]octane-8-carboxylate (210 mg, 0.34 mmol, quantitative) as a colorless oil. This was used as such for the next step without further purification; MS (ESI): m/z 610.2 [M−H]⁻.

### Step 5

### Synthesis of 2-methoxyethyl (1S,2R,5R)-3-((3-fluoro-4-((1-methyl-1H-pyrazol-4-yl)oxy)phenyl)sulfonyl)-2-(hydroxycarbamoyl)-3,8-diazabicyclo[3.2.1]octane-8-carboxylate (Example 41)

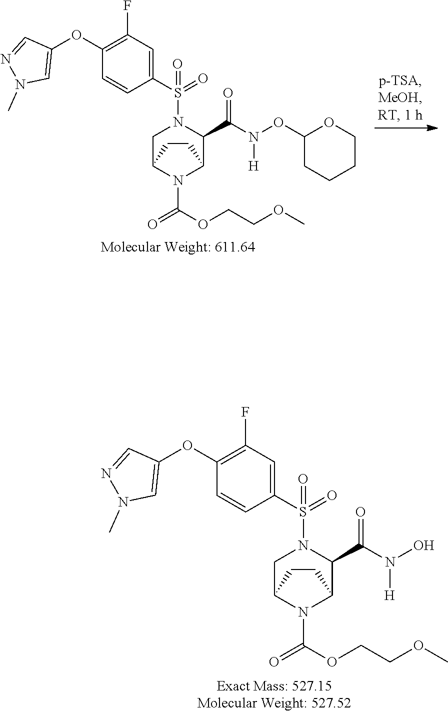 [SMILES: COCCOC(=O)N1[C@@H]2CC[C@H]1[C@H](C(=O)NOC1CCCCO1)N(S(=O)(=O)c1ccc(Oc3cnn(C)c3)c(F)c1)C2 ; COCCOC(=O)N1[C@@H]2CC[C@H]1[C@H](C(=O)NO)N(S(=O)(=O)c1ccc(Oc3cnn(C)c3)c(F)c1)C2]

Similar procedure followed as described for Step 5 of Example 24 and purification was done by RP prep HPLC (Table 2, Method 3) to obtain 2-methoxyethyl (1S,2R,5R)-3-((3-fluoro-4-((1-methyl-1H-pyrazol-4-yl)oxy)phenyl)sulfonyl)-2-(hydroxycarbamoyl)-3,8-diazabicyclo[3.2.1]octane-8-carboxylate (Example 41) (42 mg, 0.079 mmol, 19% yield for 2 steps) as an off-white solid. MS (ESI): m/z 528.2 [M+H]⁺; ¹H NMR (400 MHz, DMSO-d₆): δ 10.63 (brs, 1H), 8.76 (brs, 1H), 7.89 (s, 1H), 7.67 (d, J=10.4 Hz, 1H), 7.52-7.48 (m, 2H), 7.14 (t, J=8.4 Hz, 1H), 4.70-4.35 (m, 1H), 4.30-4.19 (m, 1H), 4.11-3.90 (m, 2H), 3.83 (s, 3H), 3.68-3.55 (m, 1H), 3.54-3.35 (m, 4H), 3.23 (s, 3H), 1.99-1.70 (m, 2H), 1.60-1.39 (m, 2H).

### Example 42

### Synthesis of 2-methoxyethyl (1S,2R,5R)-3-((3,5-difluoro-4-((1-methyl-1H-pyrazol-4-yl)oxy)phenyl)sulfonyl)-2-(hydroxycarbamoyl)-3,8-diazabicyclo[3.2.1]octane-8-carboxylate

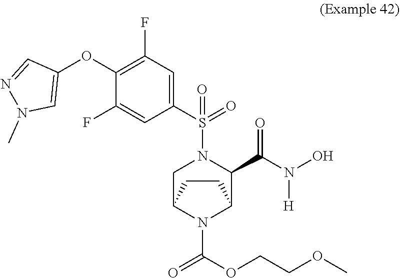 [SMILES: COCCOC(=O)N1[C@@H]2CC[C@H]1[C@H](C(=O)NO)N(S(=O)(=O)c1cc(F)c(Oc3cnn(C)c3)c(F)c1)C2]

### Step 1

### Synthesis of 2-ethyl 8-(2-methoxyethyl) (1S,2S,5R)-3-((3,4,5-trifluorophenyl)sulfonyl)-3,8-diazabicyclo[3.2.1]octane-2,8-dicarboxylate

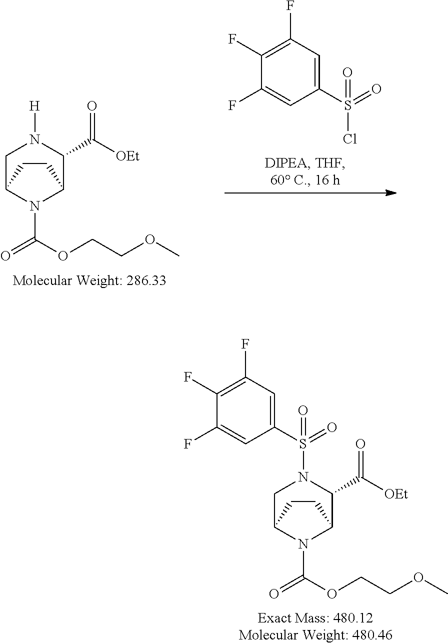 [SMILES: CCOC(=O)[C@H]1NC[C@H]2CC[C@@H]1N2C(=O)OCCOC ; O=S(=O)(Cl)c1cc(F)c(F)c(F)c1 ; CCOC(=O)[C@@H]1[C@@H]2CC[C@H](CN1S(=O)(=O)c1cc(F)c(F)c(F)c1)N2C(=O)OCCOC]

Similar procedure was followed as described for Step 1 of Example 41, to obtain 2-ethyl 8-(2-methoxyethyl) (1S,2S,5R)-3-((3,4,5-trifluorophenyl)sulfonyl)-3,8-diazabicyclo[3.2.1]octane-2,8-dicarboxylate (400 mg, 0.83 mmol, 48% yield) as a colorless gum. This was used as such for the next step without further purification. MS (ESI): m/z 481.2 [M+H]⁺.

### Step 2

### Synthesis of 2-ethyl 8-(2-methoxyethyl) (1S,2S,5R)-3-((3,5-difluoro-4-((1-methyl-1H-pyrazol-4-yl)oxy)phenyl)sulfonyl)-3,8-diazabicyclo[3.2.1]octane-2,8-dicarboxylate

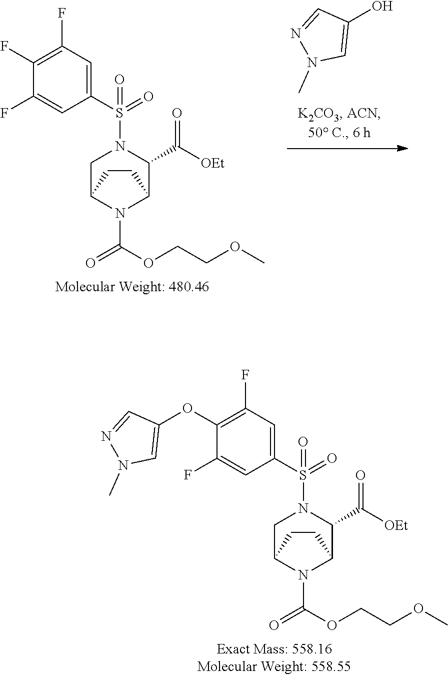

Similar procedure was followed as described for Step 2 of Example 41, to obtain 2-ethyl 8-(2-methoxyethyl) (1S,2S,5R)-3-((3,5-difluoro-4-((1-methyl-1H-pyrazol-4-yl)oxy)phenyl)sulfonyl)-3,8-diazabicyclo[3.2.1]octane-2,8-dicarboxylate (300 mg, 0.54 mmol, 64% yield) as a colorless liquid. This was used as such for the next step without further purification. MS (ESI): m/z 559.2 [M+H]⁺.

### Step 3

### Synthesis of (1S,2R,5R)-3-((3,5-difluoro-4-((1-methyl-1H-pyrazol-4-yl)oxy)phenyl)sulfonyl)-8-((2-methoxyethoxy)carbonyl)-3,8-diazabicyclo[3.2.1]octane-2-carboxylic acid

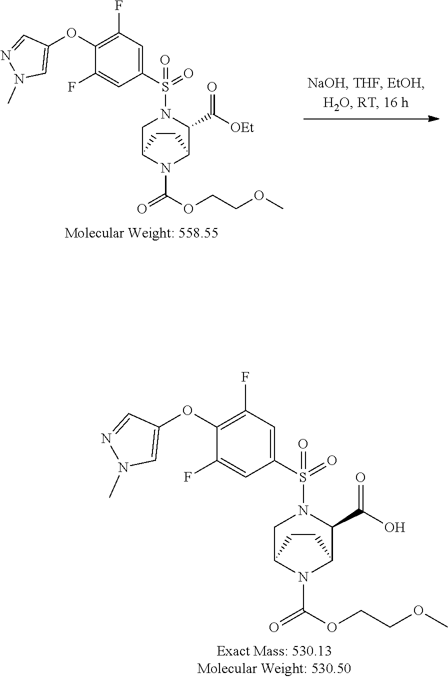 [SMILES: CCOC(=O)[C@@H]1[C@@H]2CC[C@H](CN1S(=O)(=O)c1cc(F)c(Oc3cnn(C)c3)c(F)c1)N2C(=O)OCCOC ; COCCOC(=O)N1[C@@H]2CC[C@H]1[C@H](C(=O)O)N(S(=O)(=O)c1cc(F)c(Oc3cnn(C)c3)c(F)c1)C2]

Similar procedure was followed as described for Step 4 of Example 1, to obtain (1S,2R,5R)-3-((3,5-difluoro-4-((1-methyl-1H-pyrazol-4-yl)oxy)phenyl)sulfonyl)-8-((2-methoxyethoxy)carbonyl)-3,8-diazabicyclo[3.2.1]octane-2-carboxylic acid (180 mg, 0.34 mmol, 65% yield) as a colorless gum. This was used as such for the next step without further purification. MS (ESI): m/z 531.2 [M+H]⁺.

### Step 4

### Synthesis of 2-methoxyethyl (1S,2R,5R)-3-((3,5-difluoro-4-((1-methyl-1H-pyrazol-4-yl)oxy)phenyl)sulfonyl)-2-(((tetrahydro-2H-pyran-2-yl)oxy)carbamoyl)-3,8-diazabicyclo[3.2.1]octane-8-carboxylate

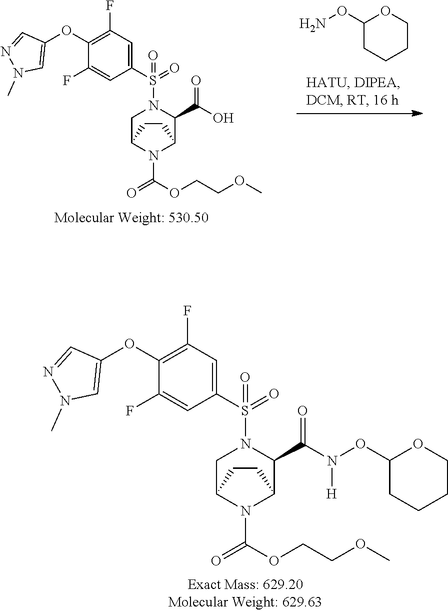 [SMILES: COCCOC(=O)N1[C@@H]2CC[C@H]1[C@H](C(=O)O)N(S(=O)(=O)c1cc(F)c(Oc3cnn(C)c3)c(F)c1)C2 ; NOC1CCCCO1 ; COCCOC(=O)N1[C@@H]2CC[C@H]1[C@H](C(=O)NOC1CCCCO1)N(S(=O)(=O)c1cc(F)c(Oc3cnn(C)c3)c(F)c1)C2]

Similar procedure was followed as described for Step 4 of Example 41, to obtain 2-methoxyethyl (1S,2R,5R)-3-((3,5-difluoro-4-((1-methyl-1H-pyrazol-4-yl)oxy)phenyl)sulfonyl)-2-(((tetrahydro-2H-pyran-2-yl)oxy)carbamoyl)-3,8-diazabicyclo[3.2.1]octane-8-carboxylate (200 mg, 0.32 mmol, quantitative) as colorless liquid. This was used as such for the next step without further purification. MS (ESI): m/z 628.2 [M−H]⁻.

### Step 5

### Synthesis of 2-methoxyethyl (1S,2R,5R)-3-((3,5-difluoro-4-((1-methyl-1H-pyrazol-4-yl)oxy)phenyl)sulfonyl)-2-(hydroxycarbamoyl)-3,8-diazabicyclo[3.2.1]octane-8-carboxylate (Example 42)

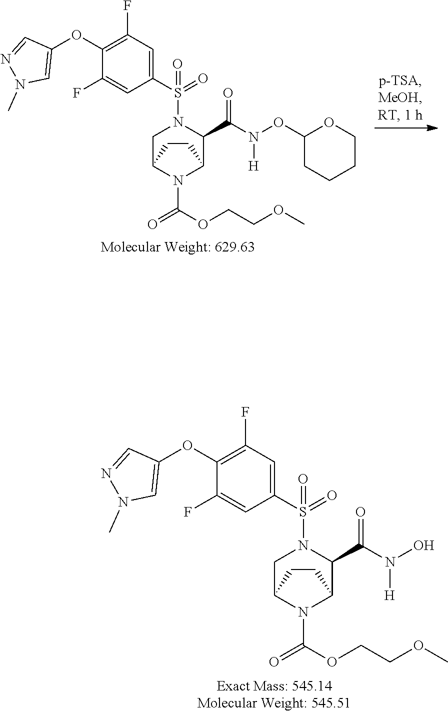 [SMILES: COCCOC(=O)N1[C@@H]2CC[C@H]1[C@H](C(=O)NOC1CCCCO1)N(S(=O)(=O)c1cc(F)c(Oc3cnn(C)c3)c(F)c1)C2 ; COCCOC(=O)N1[C@@H]2CC[C@H]1[C@H](C(=O)NO)N(S(=O)(=O)c1cc(F)c(Oc3cnn(C)c3)c(F)c1)C2]

Similar procedure followed as described for Step 5 of Example 24 and purification was done by RP prep HPLC (Table 2, Method 3) to obtain 2-methoxyethyl (1S,2R,5R)-3-((3,5-difluoro-4-((1-methyl-1H-pyrazol-4-yl)oxy)phenyl)sulfonyl)-2-(hydroxycarbamoyl)-3,8-diazabicyclo[3.2.1]octane-8-carboxylate (Example 42) (32 mg, 0.058 mmol, 18% yield for 2 steps) as an off-white solid. MS (ESI): m/z 546.2 [M+H]⁺; 1H NMR (400 MHz, DMSO-d6): δ 10.65 (brs, 1H), 8.79 (brs, 1H), 7.69 (s, 1H), 7.66-7.61 (m, 2H), 7.33 (s, 1H), 4.69-4.36 (m, 1H), 4.33-3.99 (m, 3H), 3.98-3.80 (m, 1H), 3.74 (s, 3H), 3.69-3.59 (m, 1H), 3.52-3.40 (m, 3H), 3.23 (s, 3H), 1.99-1.72 (m, 2H), 1.70-1.42 (m, 2H).

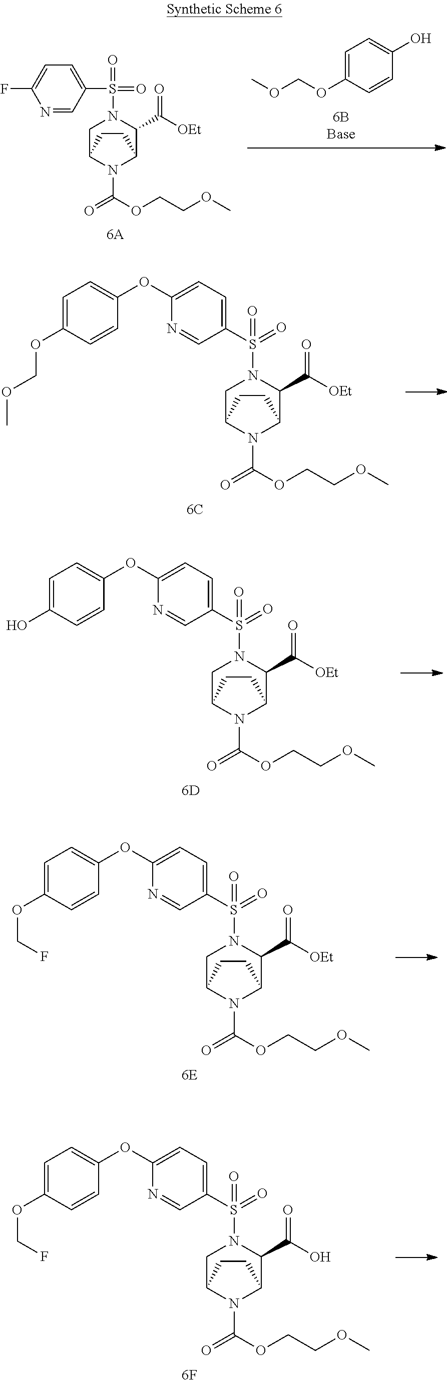 [SMILES: CCOC(=O)[C@@H]1[C@@H]2CC[C@H](CN1S(=O)(=O)c1ccc(F)nc1)N2C(=O)OCCOC ; COCOc1ccc(O)cc1 ; CCOC(=O)[C@H]1[C@@H]2CC[C@H](CN1S(=O)(=O)c1ccc(Oc3ccc(OCOC)cc3)nc1)N2C(=O)OCCOC ; CCOC(=O)[C@H]1[C@@H]2CC[C@H](CN1S(=O)(=O)c1ccc(Oc3ccc(O)cc3)nc1)N2C(=O)OCCOC ; CCOC(=O)[C@H]1[C@@H]2CC[C@H](CN1S(=O)(=O)c1ccc(Oc3ccc(OCF)cc3)nc1)N2C(=O)OCCOC ; COCCOC(=O)N1[C@@H]2CC[C@H]1[C@H](C(=O)O)N(S(=O)(=O)c1ccc(Oc3ccc(OCF)cc3)nc1)C2]

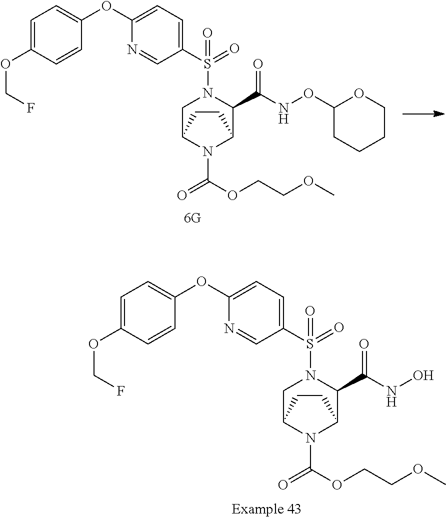 [SMILES: COCCOC(=O)N1[C@@H]2CC[C@H]1[C@H](C(=O)NOC1CCCCO1)N(S(=O)(=O)c1ccc(Oc3ccc(OCF)cc3)nc1)C2 ; COCCOC(=O)N1[C@@H]2CC[C@H]1[C@H](C(=O)NO)N(S(=O)(=O)c1ccc(Oc3ccc(OCF)cc3)nc1)C2]

### Example 43

### Synthesis of 2-methoxyethyl (1S,2R,5R)-3-((6-(4-(fluoromethoxy)phenoxy)pyridin-3-yl)sulfonyl)-2-(hydroxycarbamoyl)-3,8-diazabicyclo[3.2.1]octane-8-carboxylate

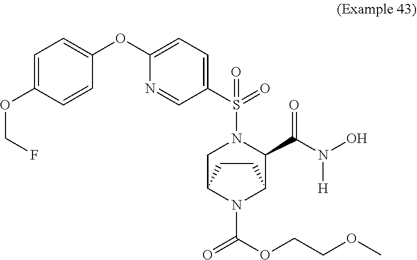 [SMILES: COCCOC(=O)N1[C@@H]2CC[C@H]1[C@H](C(=O)NO)N(S(=O)(=O)c1ccc(Oc3ccc(OCF)cc3)nc1)C2]

### Step 1

### Synthesis of 2-ethyl 8-(2-methoxyethyl) (1S,2R,5R)-3-((6-(4-(methoxymethoxy)-phenoxy)pyridin-3-yl)sulfonyl)-3,8-diazabicyclo[3.2.1]octane-2,8-dicarboxylate

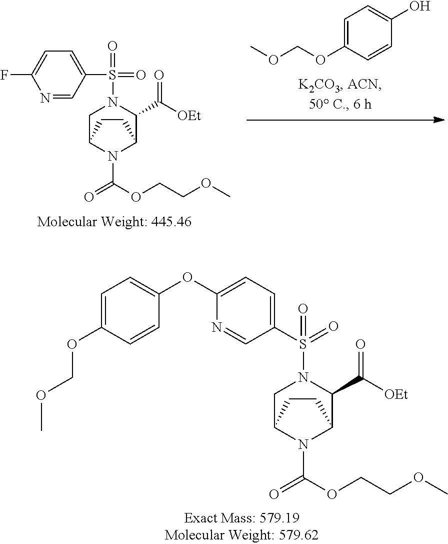 [SMILES: CCOC(=O)[C@@H]1[C@@H]2CC[C@H](CN1S(=O)(=O)c1ccc(F)nc1)N2C(=O)OCCOC ; COCOc1ccc(O)cc1 ; CCOC(=O)[C@H]1[C@@H]2CC[C@H](CN1S(=O)(=O)c1ccc(Oc3ccc(OCOC)cc3)nc1)N2C(=O)OCCOC]

To a stirred solution of 2-ethyl 8-(2-methoxyethyl) (1S,2S,5R)-3-((6-fluoropyridin-3-yl)sulfonyl)-3,8-diazabicyclo[3.2.1]octane-2,8-dicarboxylate (400 mg, 0.89 mmol) in ACN (10 mL) was added K₂CO₃ (372 mg, 2.69 mmol) followed by 4-(methoxymethoxy)phenol (207 mg, 1.35 mmol) in ACN (5 mL) at RT. The reaction was stirred at 50° C. for 6 h while being monitored by TLC. The reaction was cooled to RT, quenched with ice cold water (20 mL) and extracted with EtOAc (2×50 mL). The combined organic layer was washed with brine (20 mL), dried over Na₂SO₄, filtered and concentrated under reduced pressure to get crude product. The crude product was purified by silica gel column chromatography (100-200 mesh, 20-30% EtOAc in hexanes) to afford 2-ethyl 8-(2-methoxyethyl) (1S,2R,5R)-3-((6-(4-(methoxymethoxy)phenoxy)pyridin-3-yl)sulfonyl)-3,8-diazabicyclo[3.2.1]octane-2,8-dicarboxylate (380 mg, 0.65 mmol, 73% yield) as a light brown syrup. MS (ESI): m/z 580.3 [M+H]⁺.

### Step 2

### Synthesis of 2-ethyl 8-(2-methoxyethyl) (1S,2R,5R)-3-((6-(4-hydroxyphenoxy)pyridin-3-yl)sulfonyl)-3,8-diazabicyclo[3.2.1]octane-2,8-dicarboxylate

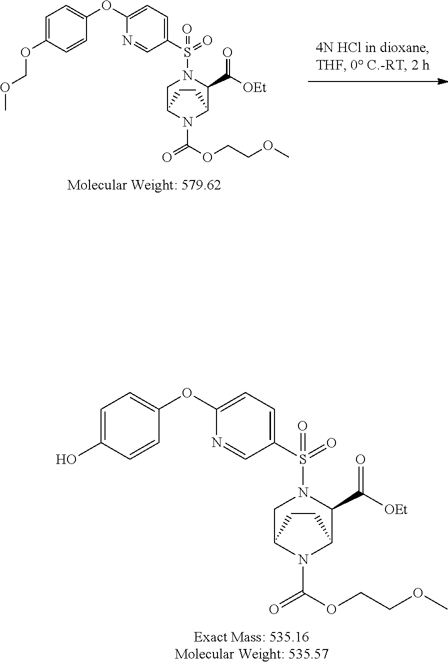 [SMILES: CCOC(=O)[C@H]1[C@@H]2CC[C@H](CN1S(=O)(=O)c1ccc(Oc3ccc(OCOC)cc3)nc1)N2C(=O)OCCOC ; CCOC(=O)[C@H]1[C@@H]2CC[C@H](CN1S(=O)(=O)c1ccc(Oc3ccc(O)cc3)nc1)N2C(=O)OCCOC]

To a stirred solution of 2-ethyl 8-(2-methoxyethyl) (1S,2R,5R)-3-((6-(4-(methoxymethoxy)phenoxy)pyridin-3-yl)sulfonyl)-3,8-diazabicyclo[3.2.1]octane-2,8-dicarboxylate (380 mg, 0.65 mmol) in THF (10 mL) at 0-10° C. was added 4M HCl in dioxane (7.6 mL) and stirred at room temperature for 2 h while being monitored by TLC. The volatiles were evaporated to dryness to obtain 2-ethyl 8-(2-methoxyethyl) (1S,2R,5R)-3-((6-(4-hydroxyphenoxy)pyridin-3-yl)sulfonyl)-3,8-diazabicyclo[3.2.1]octane-2,8-dicarboxylate (340 mg, 0.63 mmol, 97% yield) as a light brown gum. MS (ESI): m/z 536.2 [M+H]⁺.

### Step 3

### Synthesis of 2-ethyl 8-(2-methoxyethyl) (1S,2R,5R)-3-((6-(4-(fluoromethoxy)-phenoxy)pyridin-3-yl)sulfonyl)-3,8-diazabicyclo[3.2.1]octane-2,8-dicarboxylate

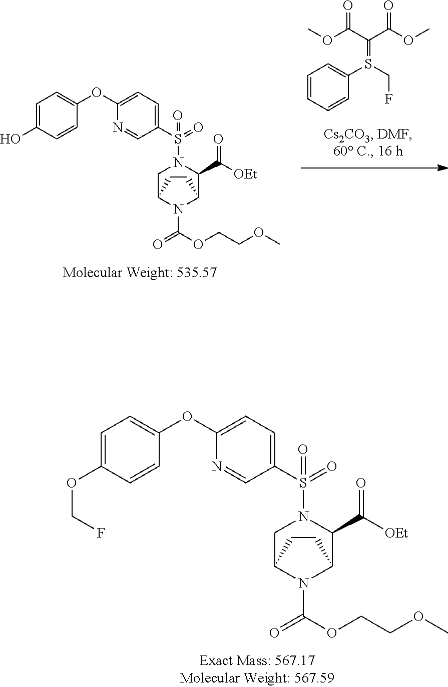 [SMILES: CCOC(=O)[C@H]1[C@@H]2CC[C@H](CN1S(=O)(=O)c1ccc(Oc3ccc(O)cc3)nc1)N2C(=O)OCCOC ; COC(=O)C(C(=O)OC)=S(CF)c1ccccc1 ; CCOC(=O)[C@H]1[C@@H]2CC[C@H](CN1S(=O)(=O)c1ccc(Oc3ccc(OCF)cc3)nc1)N2C(=O)OCCOC]

To a stirred solution of 2-ethyl 8-(2-methoxyethyl) (1S,2R,5R)-3-((6-(4-hydroxyphenoxy)pyridin-3-yl)sulfonyl)-3,8-diazabicyclo[3.2.1]octane-2,8-dicarboxylate (340 mg, 0.63 mmol) in DMF (5 mL) at 0-10° C. was added Cs₂CO₃ (414 mg, 1.27 mmol) and stirred for 10 min. Dimethyl 2-((fluoromethyl)(phenyl)-14-sulfaneylidene)malonate (259 mg, 0.95 mmol) in DMF (2 mL) was added to the reaction at same temperature. The reaction was stirred at 60° C. for 16 h while being monitored by TLC. The reaction was cooled to RT, quenched with ice cold water (15 mL) and extracted with EtOAc (2×25 mL). The combined organic layer was washed with brine (10 mL), dried over Na₂SO₄, filtered and concentrated under reduced pressure to obtain crude product. The crude product was purified by combi flash reverse phase purification (10-40% ACN in H₂O) and pure fractions were lyophilized to afford 2-ethyl 8-(2-methoxyethyl) (1S,2R,5R)-3-((6-(4-(fluoromethoxy)phenoxy)pyridin-3-yl)sulfonyl)-3,8-diazabicyclo[3.2.1]octane-2,8-dicarboxylate (180 mg, 0.32 mmol, 50% yield) as a light brown syrup. MS (ESI): m/z 568.0 [M+H]⁺.

### Step 4

### Synthesis of (1S,2R,5R)-3-((6-(4-(fluoromethoxy)phenoxy)pyridin-3-yl)sulfonyl)-8-((2-methoxyethoxy)carbonyl)-3,8-diazabicyclo[3.2.1]octane-2-carboxylic acid

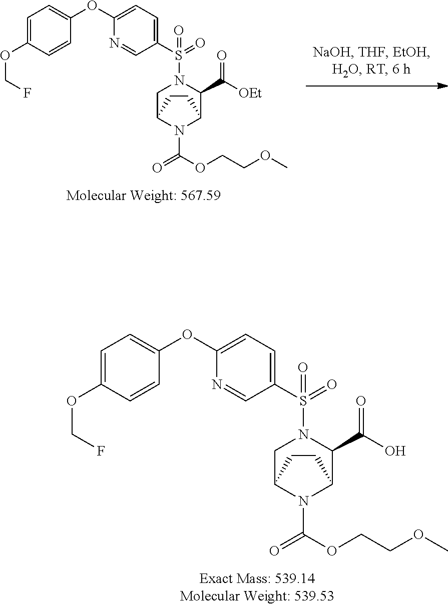 [SMILES: CCOC(=O)[C@H]1[C@@H]2CC[C@H](CN1S(=O)(=O)c1ccc(Oc3ccc(OCF)cc3)nc1)N2C(=O)OCCOC ; COCCOC(=O)N1[C@@H]2CC[C@H]1[C@H](C(=O)O)N(S(=O)(=O)c1ccc(Oc3ccc(OCF)cc3)nc1)C2]

Similar procedure was followed as described for Step 4 of Example 1, to obtain (1S,2R,5R)-3-((6-(4-(fluoromethoxy)phenoxy)pyridin-3-yl)sulfonyl)-8-((2-methoxyethoxy)carbonyl)-3,8-diazabicyclo[3.2.1]octane-2-carboxylic acid (150 mg, 0.27 mmol, 87% yield) as a brown gum. This was used as such for the next step without further purification. MS (ESI): m/z 540.2 [M+H]⁺.

### Step 5

### Synthesis of 2-methoxyethyl (1S,2R,5R)-3-((6-(4-(fluoromethoxy)phenoxy)pyridin-3-yl)sulfonyl)-2-(((tetrahydro-2H-pyran-2-yl)oxy)carbamoyl)-3,8-diazabicyclo[3.2.1]octane-8-carboxylate

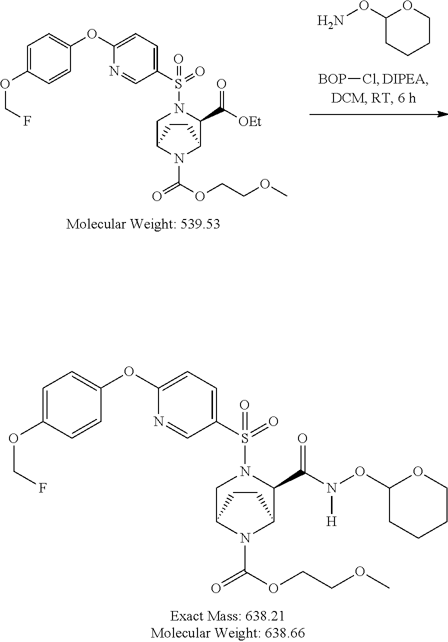

Similar procedure was followed as described for Step 3 of Example 10, to obtain 2-methoxyethyl (1S,2R,5R)-3-((6-(4-(fluoromethoxy)phenoxy)pyridin-3-yl)sulfonyl)-2-(((tetrahydro-2H-pyran-2-yl)oxy)carbamoyl)-3,8-diazabicyclo[3.2.1]octane-8-carboxylate (160 mg, 0.25 mmol, 92% yield) as a colorless syrup. This was used as such for the next step without further purification. MS (ESI): m/z 639.2 [M+H]⁺.

### Step 6

### Synthesis of 2-methoxyethyl (1S,2R,5R)-3-((6-(4-(fluoromethoxy)phenoxy)pyridin-3-yl)sulfonyl)-2-(hydroxycarbamoyl)-3,8-diazabicyclo[3.2.1]octane-8-carboxylate (Example 43)

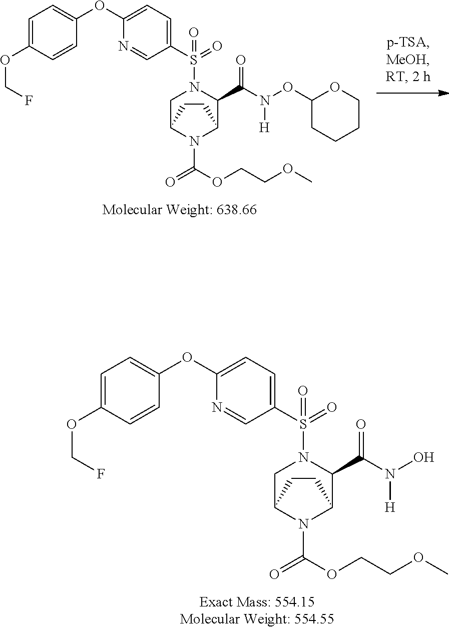 [SMILES: COCCOC(=O)N1[C@@H]2CC[C@H]1[C@H](C(=O)NOC1CCCCO1)N(S(=O)(=O)c1ccc(Oc3ccc(OCF)cc3)nc1)C2 ; COCCOC(=O)N1[C@@H]2CC[C@H]1[C@H](C(=O)NO)N(S(=O)(=O)c1ccc(Oc3ccc(OCF)cc3)nc1)C2]

Similar procedure was followed as described for Step 4 of Example 10 and purification was done by silica gel column chromatography (SiO₂, 100-200 mesh, 1-3% MeOH in DCM) to afford 2-methoxyethyl (1S,2R,5R)-3-((6-(4-(fluoromethoxy)phenoxy)pyridin-3-yl)sulfonyl)-2-(hydroxycarbamoyl)-3,8-diazabicyclo[3.2.1]octane-8-carboxylate (Example 43) (17 mg, 0.03 mmol, 12% yield) as an off-white solid. MS (ESI): m/z 555.2 [M+H]⁺; ¹H NMR (400 MHz, DMSO-d₆): δ 10.67 (brs, 1H), 8.78 (brs, 1H), 8.42 (s, 1H), 8.08 (dd, J=2.4, 8.8 Hz, 1H), 7.24-7.15 (m, 5H), 5.88 (d, J=54.4 Hz, 2H), 4.70-4.35 (m, 1H), 4.30-4.20 (m, 1H), 4.15-3.99 (m, 2H), 3.94-3.80 (m, 1H), 3.61-3.35 (m, 4H), 3.23 (s, 3H), 2.01-1.71 (m, 2H), 1.70-1.45 (m, 2H).

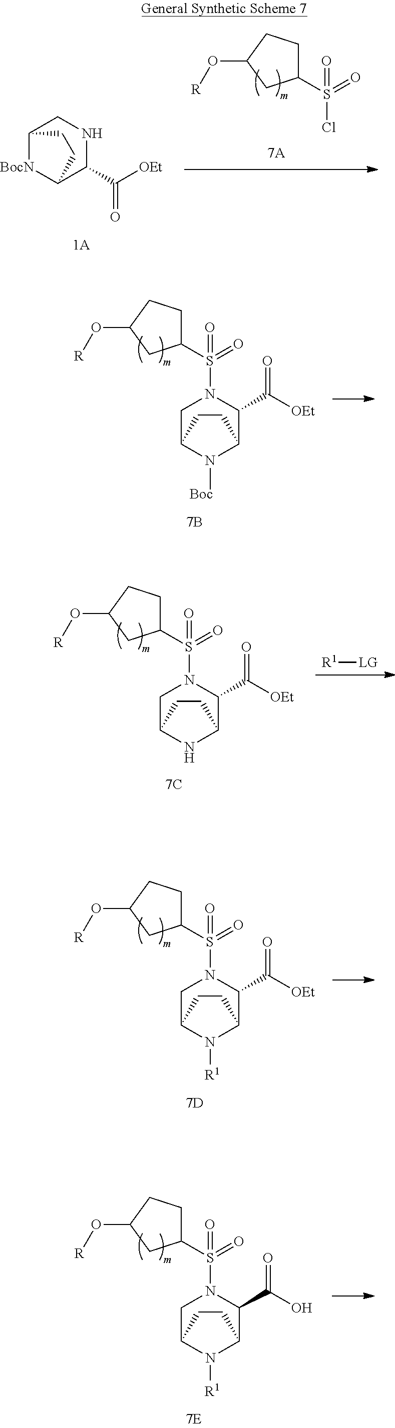

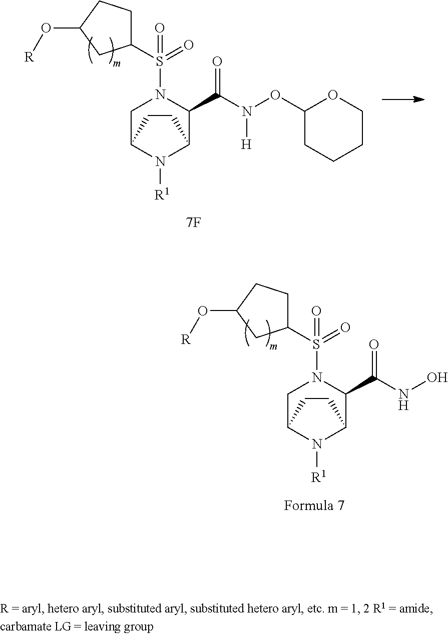

### Example 44

### Synthesis of (1S,2R,5R)-2-methoxyethyl 3-((4-(4-chlorophenoxy)piperidin-1-yl)sulfonyl)-2-(hydroxycarbamoyl)-3,8-diazabicyclo[3.2.1]octane-8-carboxylate

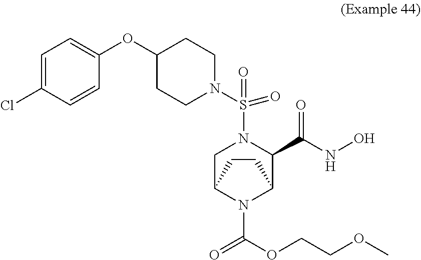 [SMILES: COCCOC(=O)N1[C@@H]2CC[C@H]1[C@H](C(=O)NO)N(S(=O)(=O)N1CCC(Oc3ccc(Cl)cc3)CC1)C2]

### Step 1

### Synthesis of (1S,2S,5R)-8-tert-butyl 2-ethyl 3-((4-(4-chlorophenoxy)piperidin-1-yl)sulfonyl)-3,8-diazabicyclo[3.2.1]octane-2,8-dicarboxylate

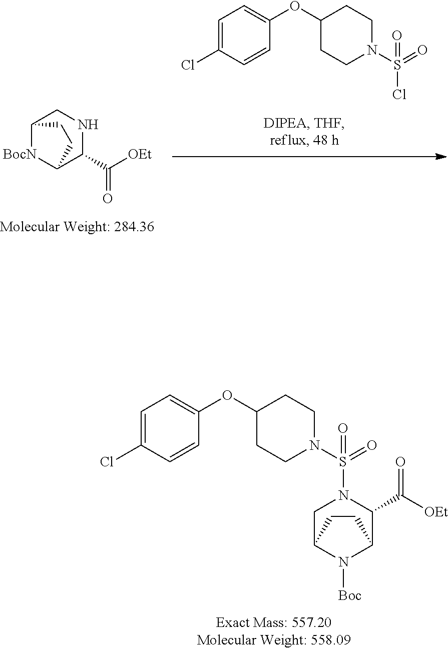 [SMILES: O=S(=O)(Cl)N1CCC(Oc2ccc(Cl)cc2)CC1 ; CCOC(=O)[C@H]1NC[C@H]2CC[C@@H]1N2C(=O)OC(C)(C)C ; CCOC(=O)[C@@H]1[C@@H]2CC[C@H](CN1S(=O)(=O)N1CCC(Oc3ccc(Cl)cc3)CC1)N2C(=O)OC(C)(C)C]

To a stirring solution of (1S,2S,5R)-8-tert-butyl 2-ethyl 3,8-diazabicyclo[3.2.1]octane-2,8-dicarboxylate (200 mg, 0.70 mmol) in THF (3 mL) under nitrogen atmosphere at 0-10° C., was added DIPEA (0.4 mL, 2.11 mmol) and stirred for 20 min. 4-(4-chlorophenoxy)piperidine-1-sulfonyl chloride (309 mg, 1.05 mmol) was dissolved in THF (2 mL) and added to the reaction mixture at the same temperature. The reaction mixture was allowed to stir at 60° C. for 48 h while being monitored by TLC. The reaction mixture was cooled to RT, diluted with water (10 mL) and extracted with ethyl acetate (2×20 mL). The combined organic layer was dried over Na₂SO₄, filtered and concentrated under reduced pressure. The crude was purified by silica gel column chromatography (100-200 mesh, 15-20% EtOAc in hexanes) to afford (1S,2S,5R)-8-tert-butyl 2-ethyl 3-((4-(4-chlorophenoxy)piperidin-1-yl)sulfonyl)-3,8-diazabicyclo[3.2.1]octane-2,8-dicarboxylate (160 mg, 0.28 mmol, 40% yield) as a colorless sticky gum. MS (ESI): m/z 558.4 [M+H]⁺.

### Step 2

### Synthesis of (1S,2S,5R)-ethyl 3-((4-(4-chlorophenoxy)piperidin-1-yl)sulfonyl)-3,8-diazabicyclo[3.2.1]octane-2-carboxylate

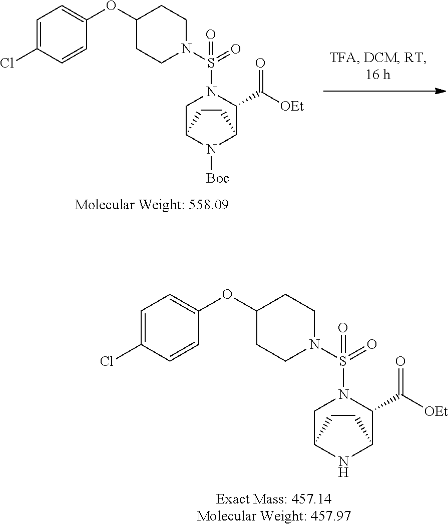 [SMILES: CCOC(=O)[C@@H]1[C@@H]2CC[C@H](CN1S(=O)(=O)N1CCC(Oc3ccc(Cl)cc3)CC1)N2C(=O)OC(C)(C)C ; CCOC(=O)[C@@H]1[C@@H]2CC[C@H](CN1S(=O)(=O)N1CCC(Oc3ccc(Cl)cc3)CC1)N2]

Similar procedure was followed as described for Step 2 of Example 1, to obtain (1S,2S,5R)-ethyl 3-((4-(4-chlorophenoxy)piperidin-1-yl)sulfonyl)-3,8-diazabicyclo[3.2.1]octane-2-carboxylate (150 mg, 0.32 mmol, quantitative) as a colorless liquid. MS (ESI): 458.4 [M+H]⁺.

### Step 3

### Synthesis of (1S,2S,5R)-2-ethyl 8-(2-methoxyethyl) 3-((4-(4-chlorophenoxy)piperidin-1-yl)sulfonyl)-3,8-diazabicyclo[3.2.1]octane-2,8-dicarboxylate

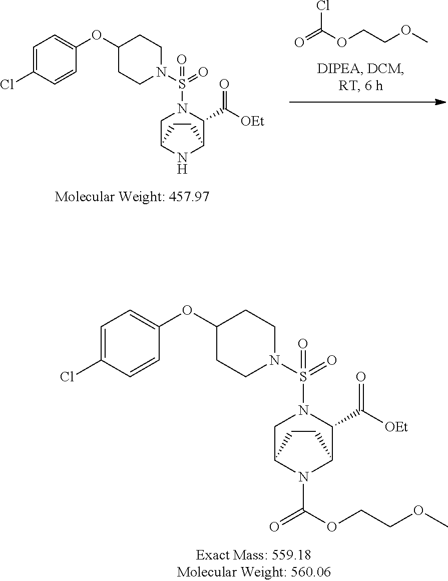

Similar procedure was followed as described for Step 3 of Example 1, to afford (1S,2S,5R)-2-ethyl 8-(2-methoxyethyl) 3-((4-(4-chlorophenoxy)piperidin-1-yl)sulfonyl)-3,8-diazabicyclo[3.2.1]octane-2,8-dicarboxylate (140 mg, 0.25 mmol, 89% yield) as a colorless sticky gum. MS (ESI): 560.4 [M+H]⁺.

### Step 4

### Synthesis of (1S,2R,5R)-3-((4-(4-chlorophenoxy)piperidin-1-yl)sulfonyl)-8-((2-methoxy-ethoxy)carbonyl)-3,8-diazabicyclo[3.2.1]octane-2-carboxylic acid

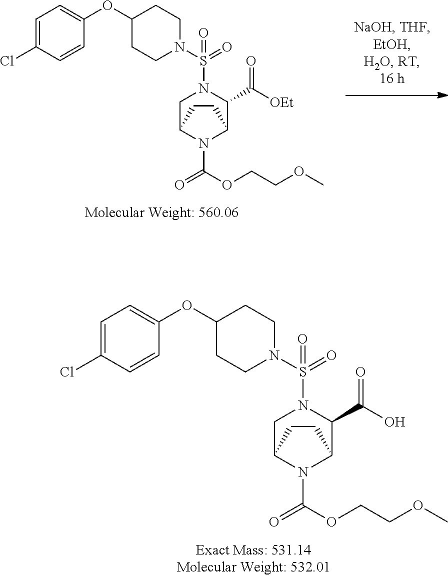 [SMILES: CCOC(=O)[C@@H]1[C@@H]2CC[C@H](CN1S(=O)(=O)N1CCC(Oc3ccc(Cl)cc3)CC1)N2C(=O)OCCOC ; COCCOC(=O)N1[C@@H]2CC[C@H]1[C@H](C(=O)O)N(S(=O)(=O)N1CCC(Oc3ccc(Cl)cc3)CC1)C2]

Similar procedure was followed as described for Step 4 of Example 1, to obtain (1S,2R,5R)-3-((4-(4-chlorophenoxy)piperidin-1-yl)sulfonyl)-8-((2-methoxyethoxy)carbonyl)-3,8-diazabicyclo[3.2.1]octane-2-carboxylic acid (90 mg, 0.17 mmol, 73% yield) as a brown syrup. MS (ESI): m/z 532.1 [M+H]⁺; ¹H NMR (400 MHz, DMSO-d₆): δ 12.94 (brs, 1H), 7.34-7.30 (m, 2H), 7.03-6.99 (m, 2H), 4.62-4.50 (m, 2H), 4.33-4.27 (m, 1H), 4.26-4.17 (m, 1H), 4.16-4.03 (m, 2H), 3.60-3.47 (m, 4H), 3.26 (s, 3H), 3.25-3.15 (m, 1H), 3.12-3.04 (m, 2H), 2.01-1.80 (m, 5H), 1.76-1.60 (m, 4H).

### Step 5

### Synthesis of (1S,2R,5R)-2-methoxyethyl 3-((4-(4-chlorophenoxy)piperidin-1-yl)sulfonyl)-2-(((tetrahydro-2H-pyran-2-yl)oxy)carbamoyl)-3,8-diazabicyclo[3.2.1]octane-8-carboxylate

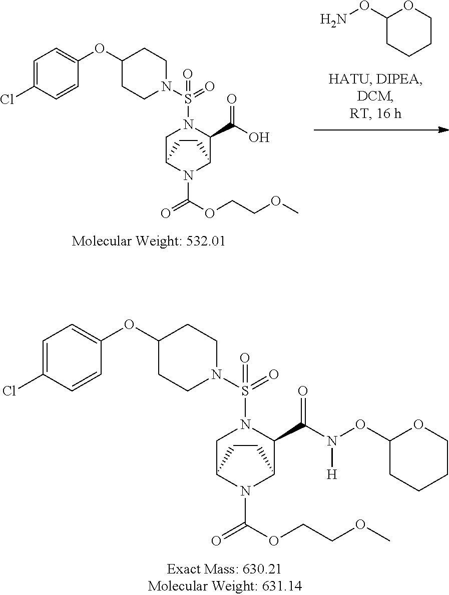 [SMILES: COCCOC(=O)N1[C@@H]2CC[C@H]1[C@H](C(=O)O)N(S(=O)(=O)N1CCC(Oc3ccc(Cl)cc3)CC1)C2 ; NOC1CCCCO1 ; COCCOC(=O)N1[C@@H]2CC[C@H]1[C@H](C(=O)NOC1CCCCO1)N(S(=O)(=O)N1CCC(Oc3ccc(Cl)cc3)CC1)C2]

Similar procedure was followed as described for Step 5 of Example 1, to obtain (1S,2R,5R)-2-methoxyethyl 3-((4-(4-chlorophenoxy)piperidin-1-yl)sulfonyl)-2-(((tetrahydro-2H-pyran-2-yl)oxy)carbamoyl)-3,8-diazabicyclo[3.2.1]octane-8-carboxylate (70 mg, 0.11 mmol, quantitative) as a brown sticky gum. MS (ESI): m/z 631.2 [M+H]⁺.

### Step 6

### Synthesis of (1S,2R,5R)-2-methoxyethyl 3-((4-(4-chlorophenoxy)piperidin-1-yl)sulfonyl)-2-(hydroxycarbamoyl)-3,8-diazabicyclo[3.2.1]octane-8-carboxylate (Example 44) C₃

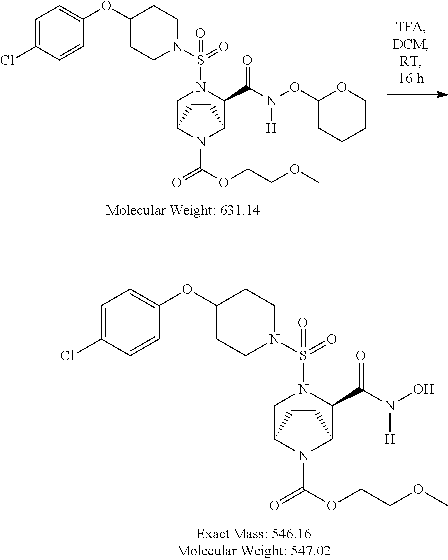 [SMILES: COCCOC(=O)N1[C@@H]2CC[C@H]1[C@H](C(=O)NOC1CCCCO1)N(S(=O)(=O)N1CCC(Oc3ccc(Cl)cc3)CC1)C2 ; COCCOC(=O)N1[C@@H]2CC[C@H]1[C@H](C(=O)NO)N(S(=O)(=O)N1CCC(Oc3ccc(Cl)cc3)CC1)C2]

Similar procedure followed as described for Step 6 of Example 1 and purification was done by RP prep HPLC (Table 2, Method 4) to obtain (1S,2R,5R)-2-methoxyethyl 3-((4-(4-chlorophenoxy)piperidin-1-yl)sulfonyl)-2-(hydroxycarbamoyl)-3,8-diazabicyclo[3.2.1]octane-8-carboxylate (Example 44) (5 mg, 0.009 mmol, 10% yield) as an off-white solid. MS (ESI): m/z 547.2 [M+H]⁺; ¹H NMR (400 MHz, DMSO-d₆): δ 10.66 (brs, 1H), 8.84-8.80 (m, 1H), 7.33-7.30 (m, 2H), 7.02-6.99 (m, 2H), 4.60-4.47 (m, 2H), 4.31-4.26 (m, 2H), 4.24-4.07 (m, 2H), 3.92-3.78 (m, 2H), 3.53-3.43 (m, 3H), 3.25 (s, 3H), 3.17-3.14 (m, 1H), 3.03-2.98 (m, 2H), 2.01-1.84 (m, 4H), 1.69-1.60 (in, 4H).

Similarly, below examples were prepared by following the same or analogous process as described in Example 44.

TABLE 8

| Ex. | Structure | Name | Analytical data |
|---|---|---|---|
| 45 | 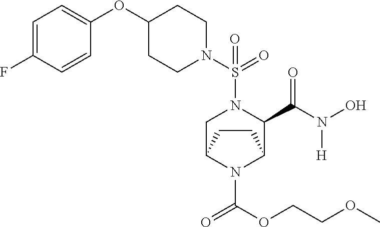 [SMILES: COCCOC(=O)N1[C@@H]2CC[C@H]1[C@H](C(=O)NO)N(S(=O)(=O)N1CCC(Oc3ccc(F)cc3)CC1)C2] | 2-methoxyethyl (1S,2R,5R)-3- ((4-(4-fluoro- phenoxy)- piperidin-1-yl)- sulfonyl)-2- (hydroxy- carbamoyl)-3,8- diazabicyclo- [3.2.1]octane-8- carboxylate | ¹H NMR (400 MHZ, DMSO- d₆): δ 10.74 (brs, 1H), 8.45 (brs, 1H), 7.13-7.08 (m, 2H), 7.02-6.99 (m, 2H), 4.50- 4.35 (m, 2H), 4.35-4.02 (m, 3H), 3.95-3.75 (m, 1H), 3.64- 3.40 (m, 4H), 3.25 (s, split, 3H), 3.22-2.98 (m, 5H), 2.00- 1.77 (m, 3H), 1.75-1.55 (m, 4H); MS (ESI): m/z 531.2 [M + H]⁺. |
| 46 | 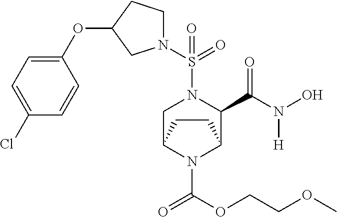 [SMILES: COCCOC(=O)N1[C@@H]2CC[C@H]1[C@H](C(=O)NO)N(S(=O)(=O)N1CCC(Oc3ccc(Cl)cc3)C1)C2] | 2-methoxyethyl (1S,2R,5R)-3- ((3-(4-chloro- phenoxy)- pyrrolidin-1-yl)- sulfonyl)-2- (hydroxy- carbamoyl)-3,8- diazabicyclo- [3.2.1]octane-8- carboxylate | ¹H NMR (400 MHZ, DMSO- d₆): δ 10.10 (brs, 1H), 8.86 (brs, 1H), 7.36-7.29 (m, 2H), 6.99-6.95 (m, 2H), 5.09- 5.00 (m, 1H), 4.50-4.00 (m, 5H), 3.99-3.70 (m, 2H), 3.61- 3.40 (m, 4H), 3.18 (s, 3H), 3.20-3.00 (m, 1H), 2.25- 2.10 (m, 1H), 2.10-1.40 (m, 6H); MS (ESI): 533.2 [M + H]⁺. |
| 47 | 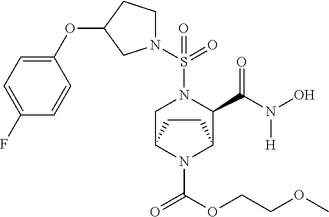 [SMILES: COCCOC(=O)N1[C@@H]2CC[C@H]1[C@H](C(=O)NO)N(S(=O)(=O)N1CCC(Oc3ccc(F)cc3)C1)C2] | 2-methoxyethyl (1S,2R,5R)-3- ((3-(4-fluoro- phenoxy)- pyrrolidin-1-yl)- sulfonyl)-2- (hydroxy- carbamoyl)-3,8- diazabicyclo- [3.2.1]octane-8- carboxylate | ¹H NMR (400 MHZ, DMSO- d₆): δ 10.76 (brs, 1H), 8.86 (brs, 1H), 7.16-7.10 (m, 2H), 6.99-6.94 (m, 2H), 5.05- 4.91 (m, 1H), 4.50-4.03 (m, 4H), 3.99-3.70 (m, 1H), 3.65- 3.35 (m, 6H), 3.26 (s, 3H), 3.18-3.05 (m, 2H), 2.45- 2.40 (m, 1H), 2.21-1.95 (m, 2H), 1.95-1.51 (m, 3H); MS (ESI): 517.2 [M + H]⁺. |
| 48 | 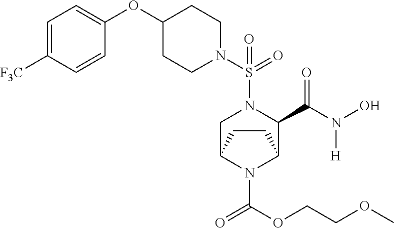 [SMILES: COCCOC(=O)N1[C@@H]2CC[C@H]1[C@H](C(=O)NO)N(S(=O)(=O)N1CCC(Oc3ccc(C(F)(F)F)cc3)CC1)C2] | 2-methoxyethyl (1S,2R,5R)-2- (hydroxy- carbamoyl)-3- ((4-(4- (trifluoromethyl) phenoxy)- piperidin-1-yl)- sulfonyl)-3,8- diazabicyclo- [3.2.1]octane-8- carboxylate | ¹H NMR (400 MHZ, DMSO- d₆): δ 10.75 (brs, 1H), 8.85 (brs, 1H), 7.65-7.62 (m, 2H), 7.18-7.15 (m, 2H), 4.72- 4.60 (m, 1H), 4.49-4.05 (m, 4H), 4.01-3.75 (m, 1H), 3.63- 3.46 (m, 4H), 3.27 (s, splitted, 3H), 3.22-3.01 (m, 4H), 2.04-1.75 (m, 4H), 1.74- 1.54 (m, 4H); MS (ESI): m/z 581.3 [M + H]⁺. |
| 49 | 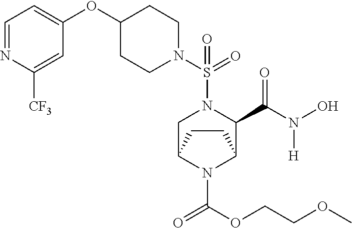 [SMILES: COCCOC(=O)N1[C@@H]2CC[C@H]1[C@H](C(=O)NO)N(S(=O)(=O)N1CCC(Oc3ccnc(C(F)(F)F)c3)CC1)C2] | 2-methoxyethyl (1S,2R,5R)-2- (hydroxy- carbamoyl)-3- ((4-((2- (trifluoro- methyl)pyridin- 4-yl)oxy)- piperidin-1-yl)- sulfonyl)-3,8- diazabicyclo- [3.2.1]octane-8- carboxylate | ¹H NMR (400 MHZ, DMSO- d₆): δ 10.61 (brs, 1H), 8.79 (brs, 1H), 8.56 (d, J = 6.0 Hz, 1H), 7.47 (d, J = 2.0 Hz, 1H), 7.33-7.31 (m, 1H), 4.90- 4.80 (m, 1H), 4.70-4.32 (m, 1H), 4.31-4.20 (m, 1H), 4.15- 4.00 (m, 2H), 3.99-3.76 (m, 2H), 3.60-3.41 (m, 2H), 3.41- 3.38 (m, 3H), 3.22 (s, 3H), 3.12-3.05 (m, 2H), 2.04 - 1.75 (m, 4H), 1.74-1.60 (m, 4H); MS (ESI): m/z 582.2 [M + H]⁺. |
| 50 | 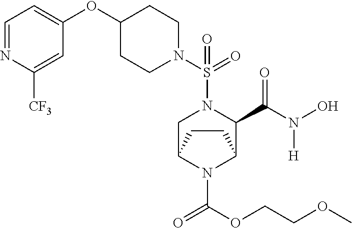 | ethyl (1S,2R,5R)-2- (hydroxy- carbamoyl)-3- ((4-((2- (trifluoro- methyl)pyridin- 4-yl)oxy)- piperidin-1-yl)- sulfonyl)-3,8- diazabicyclo- [3.2.1]octane-8- carboxylate | ¹H NMR (400 MHz, DMSO- d₆): δ 10.75 (brs, 1H), 8.86 (brs, 1H), 8.57-8.55 (m, 1H), 7.48-7.46 (m, 1H), 7.34- 7.32 (m, 1H), 4.90-4.80 (m, 1H), 4.70-4.35 (m, 1H), 4.35- 4.18 (m, 1H), 4.13-3.76 (m, 4H), 3.61-3.46 (m, 2H), 3.25- 3.01 (m, 3H), 2.09-1.76 (m, 4H), 1.74-1.56 (m, 4H), 1.23- 1.14 (m, 3H); MS (ESI): m/z 552.2 [M + H]⁺. |

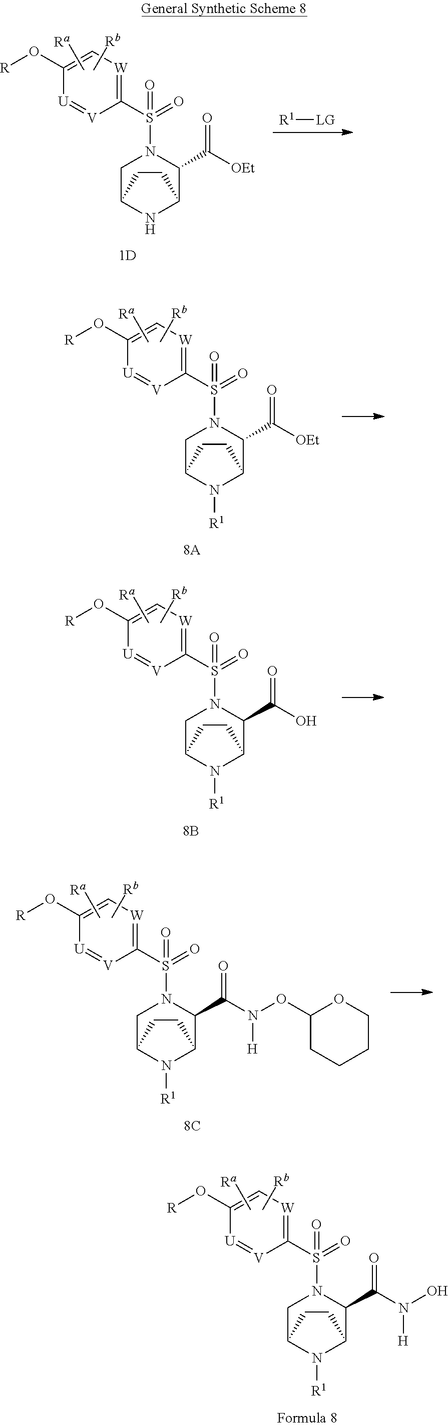

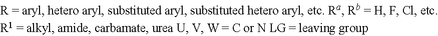

### Example 51

### Synthesis of (1S,2R,5R)-8-(2-(dimethylamino)-2-oxoethyl)-3-((6-(4-fluorophenoxy)pyridin-3-yl)sulfonyl)-N-hydroxy-3,8-diazabicyclo[3.2.1]octane-2-carboxamide

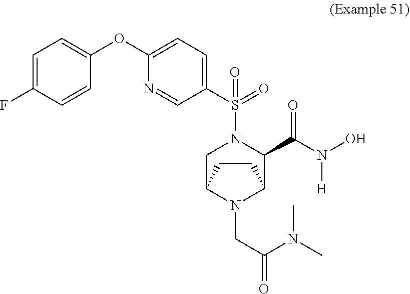 [SMILES: CN(C)C(=O)CN1[C@@H]2CC[C@H]1[C@H](C(=O)NO)N(S(=O)(=O)c1ccc(Oc3ccc(F)cc3)nc1)C2]

### Step 1

### Synthesis of ethyl (1S,2S,5R)-8-(2-(dimethylamino)-2-oxoethyl)-3-((6-(4-fluorophenoxy)pyridin-3-yl)sulfonyl)-3,8-diazabicyclo[3.2.1]octane-2-carboxylate

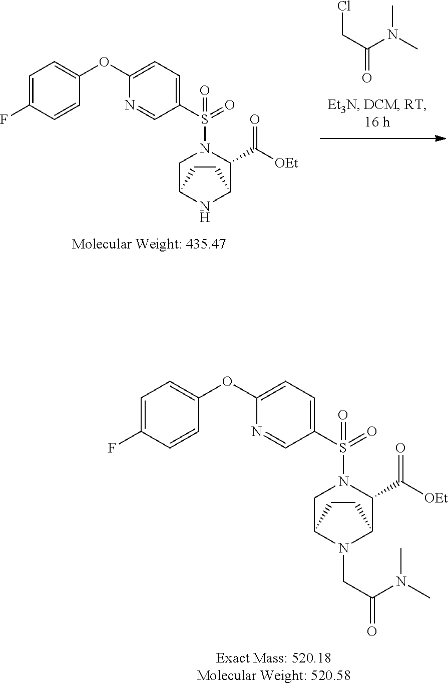

To a stirred solution of ethyl (1S,2S,5R)-3-((6-(4-fluorophenoxy)pyridin-3-yl)sulfonyl)-3,8-diazabicyclo[3.2.1]octane-2-carboxylate (200 mg, 0.46 mmol) in DCM (5 mL) at 0-10° C. was added Et₃N (0.2 mL, 1.83 mmol) and stirred for 10 min at same temperature. 2-Chloro-N,N-dimethylacetamide (55 mg, 0.46 mmol) was added and the reaction was stirred at RT for 16 h while being monitored by TLC. The reaction was quenched by ice cold water (5 mL) and extracted with DCM (2×20 mL). The organic layer was separated, dried over anhydrous Na₂SO₄, filtered and concentrated under reduced pressure to afford ethyl (1S,2S,5R)-8-(2-(dimethylamino)-2-oxoethyl)-3-((6-(4-fluorophenoxy)pyridin-3-yl)sulfonyl)-3,8-diazabicyclo[3.2.1]octane-2-carboxylate (180 mg, 0.34 mmol, 74% yield) as a light yellow gum. This was used as such for the next step without further purification. MS (ESI): m/z 521.1 [M+H]⁺.

### Step 2

### Synthesis of (1S,2R,5R)-8-(2-(dimethylamino)-2-oxoethyl)-3-((6-(4-fluorophenoxy)pyridin-3-yl)sulfonyl)-3,8-diazabicyclo[3.2.1]octane-2-carboxylic acid

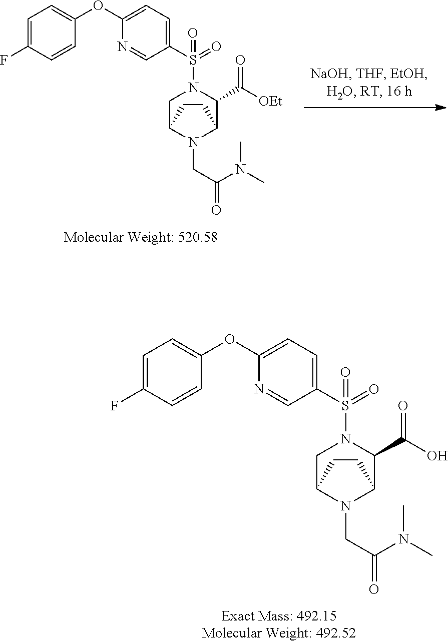 [SMILES: CCOC(=O)[C@@H]1[C@@H]2CC[C@H](CN1S(=O)(=O)c1ccc(Oc3ccc(F)cc3)nc1)N2CC(=O)N(C)C ; CN(C)C(=O)CN1[C@@H]2CC[C@H]1[C@H](C(=O)O)N(S(=O)(=O)c1ccc(Oc3ccc(F)cc3)nc1)C2]

Similar procedure was followed as described for Step 4 of Example 1, to obtain (1S,2R,5R)-8-(2-(dimethylamino)-2-oxoethyl)-3-((6-(4-fluorophenoxy)pyridin-3-yl)sulfonyl)-3,8-diazabicyclo[3.2.1]octane-2-carboxylic acid (90 mg, 0.18 mmol) as a light brown sticky gum. This was used as such for the next step without further purification. MS (ESI): m/z 493.2 [M+H]⁺.

### Step 3

### Synthesis of (1S,2R,5R)-8-(2-(dimethylamino)-2-oxoethyl)-3-((6-(4-fluorophenoxy)pyridin-3-yl)sulfonyl)-N-((tetrahydro-2H-pyran-2-yl)oxy)-3,8-diazabicyclo[3.2.1]octane-2-carboxamide

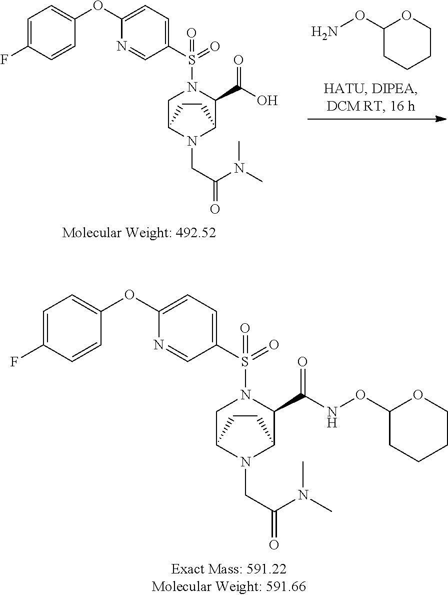 [SMILES: CN(C)C(=O)CN1[C@@H]2CC[C@H]1[C@H](C(=O)O)N(S(=O)(=O)c1ccc(Oc3ccc(F)cc3)nc1)C2 ; NOC1CCCCO1 ; CN(C)C(=O)CN1[C@@H]2CC[C@H]1[C@H](C(=O)NOC1CCCCO1)N(S(=O)(=O)c1ccc(Oc3ccc(F)cc3)nc1)C2]

Similar procedure was followed as described for Step 5 of Example 1, to obtain (1S,2R,5R)-8-(2-(dimethylamino)-2-oxoethyl)-3-((6-(4-fluorophenoxy)pyridin-3-yl)sulfonyl)-N-((tetrahydro-2H-pyran-2-yl)oxy)-3,8-diazabicyclo[3.2.1]octane-2-carboxamide (100 mg, 0.17 mmol) as a light yellow gum. This was used as such for the next step without further purification. MS (ESI): m/z 592.3 [M+H]⁺.

### Step 4

### Synthesis of (1S,2R,5R)-8-(2-(dimethylamino)-2-oxoethyl)-3-((6-(4-fluorophenoxy)pyridin-3-yl)sulfonyl)-N-hydroxy-3,8-diazabicyclo[3.2.1]octane-2-carboxamide (Example 51)

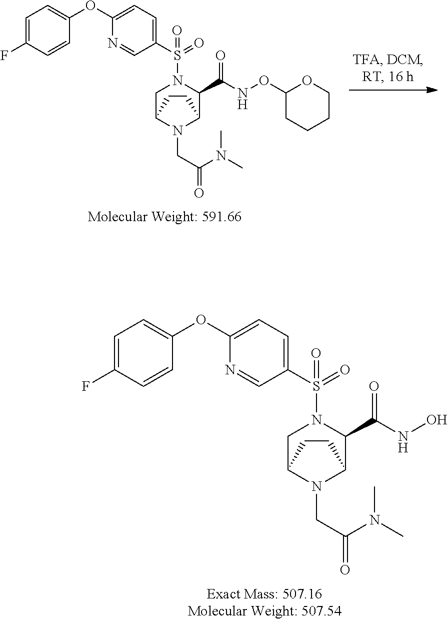 [SMILES: CN(C)C(=O)CN1[C@@H]2CC[C@H]1[C@H](C(=O)NOC1CCCCO1)N(S(=O)(=O)c1ccc(Oc3ccc(F)cc3)nc1)C2 ; CN(C)C(=O)CN1[C@@H]2CC[C@H]1[C@H](C(=O)NO)N(S(=O)(=O)c1ccc(Oc3ccc(F)cc3)nc1)C2]

Similar procedure was followed as described for Step 6 of Example 1 and purification was done by RP prep HPLC (Table 2, Method 1) to obtain (1S,2R,5R)-8-(2-(dimethylamino)-2-oxoethyl)-3-((6-(4-fluorophenoxy)pyridin-3-yl)sulfonyl)-N-hydroxy-3,8-diazabicyclo[3.2.1]octane-2-carboxamide (Example 51) (7.5 mg, 0.015 mmol, 8% yield for 2 steps) as an off white solid. MS (ESI): m/z 508.2 [M+H]⁺; ¹H NMR (400 MHz, DMSO-d6): δ 11.38 (brs, 1H), 8.67 (brs, 1H), 8.49 (d, J=2.0 Hz, 1H), 8.20 (dd, J=2.4, 8.8 Hz, 1H), 7.31-6.95 (m, 6H), 4.20-4.19 (m, 1H), 3.59-3.50 (m, 1H), 3.41-3.35 (m, 1H), 3.26-3.15 (m, 2H), 3.09-2.95 (m, 2H), 2.92 (m, 3H), 2.82 (m, 3H), 2.02-1.70 (m, 2H), 1.60-1.40 (m, 2H).

Similarly, below example was prepared by following the same or analogous process as described in Example 51.

TABLE 9

| Ex. | Structure | Name | Analytical data |
|---|---|---|---|
| 52 | 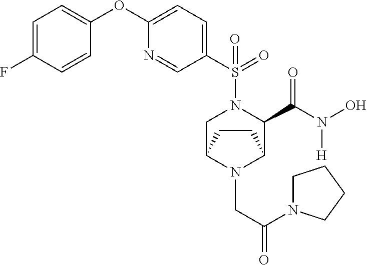 [SMILES: O=C(NO)[C@H]1[C@@H]2CC[C@H](CN1S(=O)(=O)c1ccc(Oc3ccc(F)cc3)nc1)N2CC(=O)N1CCCC1] | (1S,2R,5R)-3-((6-(4- fluorophenoxy)pyridi n-3-yl)sulfonyl)-N- hydroxy-8-(2-oxo-2- (pyrrolidin-1-yl)- ethyl)-3,8-diaza- bicyclo[3.2.1]octane- 2-carboxamide | ¹H NMR (400 MHZ, DMSO- d₆): δ 11.55 (brs, 1H), 8.66 (brs, 1H), 8.54-8.49 (m, 1H), 8.25-8.18 (m, 1H), 7.34- 7.21 (m, 4H), 7.17 (d, J = 8.8 Hz, 1H), 4.21-4.19 (m, 1H), 3.60-3.55 (m, 1H), 3.45- 3.35 (m, 3H), 3.29-3.14 (m, 4H), 3.05-2.92 (m, 2H), 2.00- 1.69 (m, 6H), 1.60-1.48 (m, 2H); MS (ESI): m/z 534.3 [M + H]⁺. |

### Example 53

### Synthesis of (1S,2R,5R)-8-acetyl-3-((6-(4-fluorophenoxy)pyridin-3-yl)sulfonyl)-N-hydroxy-3,8-diazabicyclo[3.2.1]octane-2-carboxamide

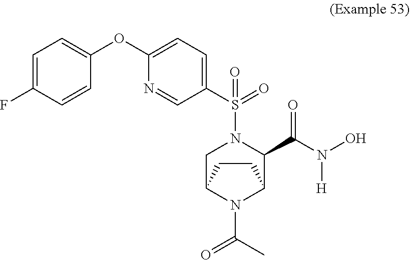 [SMILES: CC(=O)N1[C@@H]2CC[C@H]1[C@H](C(=O)NO)N(S(=O)(=O)c1ccc(Oc3ccc(F)cc3)nc1)C2]

### Step 1

### Synthesis of methyl (1S,2S,5R)-8-acetyl-3-((6-(4-fluorophenoxy)pyridin-3-yl)sulfonyl)-3,8-diazabicyclo[3.2.1]octane-2-carboxylate

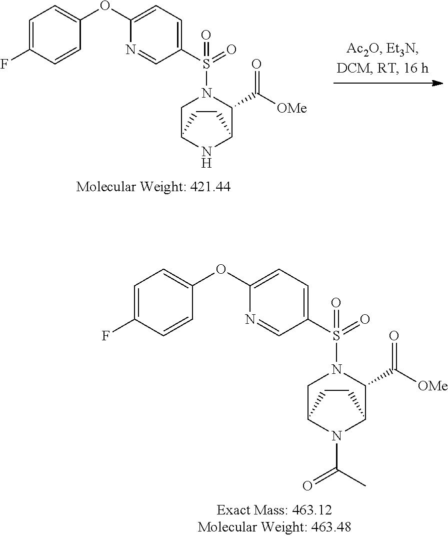 [SMILES: COC(=O)[C@@H]1[C@@H]2CC[C@H](CN1S(=O)(=O)c1ccc(Oc3ccc(F)cc3)nc1)N2 ; COC(=O)[C@@H]1[C@@H]2CC[C@H](CN1S(=O)(=O)c1ccc(Oc3ccc(F)cc3)nc1)N2C(C)=O]

To a solution of methyl (1S,2S,5R)-3-((6-(4-fluorophenoxy)pyridin-3-yl)sulfonyl)-3,8-diazabicyclo[3.2.1]octane-2-carboxylate (150 mg, 0.34 mmol; prepared in analogous fashion as the ethyl ester) in DCM (5 mL) at 0-10° C. was added Et₃N (0.14 mL, 1.03 mmol) and stirred for 10 min at same temperature. Acetic anhydride (0.06 mL, 0.68 mmol) was added and the reaction mixture was stirred at RT for 16 h while being monitored by TLC. The reaction was quenched with ice cold water (5 mL) and extracted with DCM (2×20 mL). The organic layer was separated, dried over anhydrous Na₂SO₄, filtered and concentrated under reduced pressure to afford methyl (1S,2S,5R)-8-acetyl-3-((6-(4-fluorophenoxy)pyridin-3-yl)sulfonyl)-3,8-diazabicyclo[3.2.1]octane-2-carboxylate (160 mg, 0.33 mmol, quantitative) as a light yellow syrup. This was used as such for the next step without further purification. MS (ESI): m/z 464.1 [M+H]⁺.

### Step 2

### Synthesis of (1S,2R,5R)-8-acetyl-3-((6-(4-fluorophenoxy)pyridin-3-yl)sulfonyl)-3,8-diazabicyclo[3.2.1]octane-2-carboxylic acid

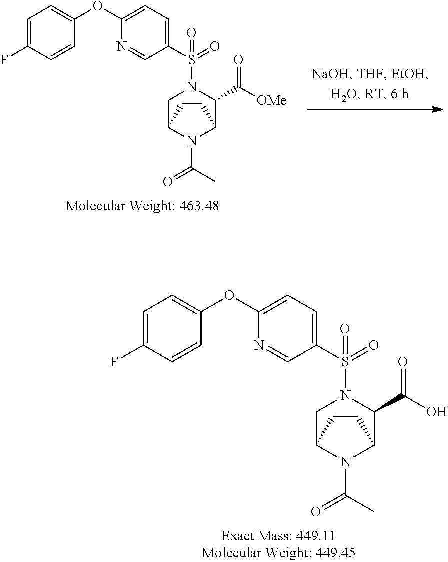 [SMILES: COC(=O)[C@@H]1[C@@H]2CC[C@H](CN1S(=O)(=O)c1ccc(Oc3ccc(F)cc3)nc1)N2C(C)=O ; CC(=O)N1[C@@H]2CC[C@H]1[C@H](C(=O)O)N(S(=O)(=O)c1ccc(Oc3ccc(F)cc3)nc1)C2]

Similar procedure was followed as described for Step 4 of Example 1, to obtain (1S,2R,5R)-8-acetyl-3-((6-(4-fluorophenoxy)pyridin-3-yl)sulfonyl)-3,8-diazabicyclo[3.2.1]octane-2-carboxylic acid (110 mg, 0.24 mmol, 77% yield) as a light brown gum. This was used as such for the next step without further purification. MS (ESI): m/z 448.1 [M−H]⁻.

### Step 3

### Synthesis of (1S,2R,5R)-8-acetyl-3-((6-(4-fluorophenoxy)pyridin-3-yl)sulfonyl)-N-((tetrahydro-2H-pyran-2-yl)oxy)-3,8-diazabicyclo[3.2.1]octane-2-carboxamide

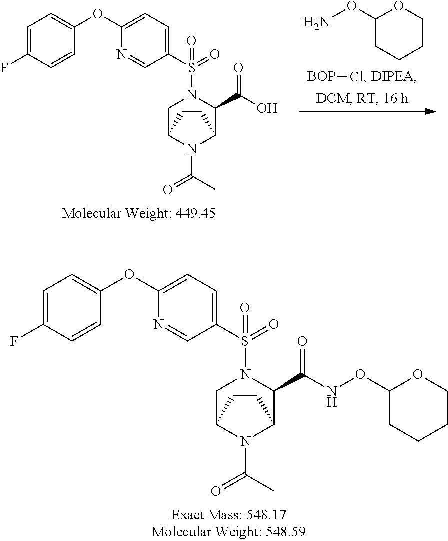

Similar procedure was followed as described for Step 3 of Example 10, to obtain (1S,2R,5R)-8-acetyl-3-((6-(4-fluorophenoxy)pyridin-3-yl)sulfonyl)-N-((tetrahydro-2H-pyran-2-yl)oxy)-3,8-diazabicyclo[3.2.1]octane-2-carboxamide (100 mg, 0.18 mmol) as a yellow gum. This was used as such for the next step without further purification. MS (ESI): m/z 547.1 [M−H]⁻.

### Step 4

### Synthesis of (1S,2R,5R)-8-acetyl-3-((6-(4-fluorophenoxy)pyridin-3-yl)sulfonyl)-N-hydroxy-3,8-diazabicyclo[3.2.1]octane-2-carboxamide (Example 53)

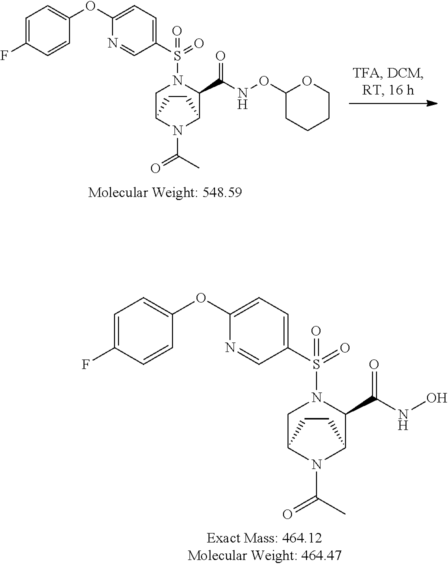 [SMILES: CC(=O)N1[C@@H]2CC[C@H]1[C@H](C(=O)NOC1CCCCO1)N(S(=O)(=O)c1ccc(Oc3ccc(F)cc3)nc1)C2 ; CC(=O)N1[C@@H]2CC[C@H]1[C@H](C(=O)NO)N(S(=O)(=O)c1ccc(Oc3ccc(F)cc3)nc1)C2]

Similar procedure was followed as described for Step 6 of Example 1 and purification was done by RP prep HPLC (Table 2, Method 4) to obtain (1S,2R,5R)-8-acetyl-3-((6-(4-fluorophenoxy)pyridin-3-yl)sulfonyl)-N-hydroxy-3,8-diazabicyclo[3.2.1]octane-2-carboxamide (Example 53) (20 mg, 0.04 mmol, 24% yield for 2 steps) as an off white solid. MS (ESI): m/z 465.2 [M+H]⁺; ¹H NMR (400 MHz, DMSO-d₆). δ 9.60 (brs, 1H), 8.80 (brs, 1H), 8.45-8.42 (m, 1H), 8.13-8.08 (m, 1H), 7.31-7.22 (m, 4H), 7.22-7.17 (m, 1H), 4.49-4.55 (m, 1H), 4.44-4.30 (m, 1H), 4.14-4.08 (m, 1H), 3.60-3.35 (m, 2H), 2.05-1.90 (m, 1H), 1.90-1.40 (m, 6H).

Similarly, below examples were prepared by following the same or analogous process as described in Example 53.

TABLE 10

| Ex. | Structure | Name | Analytical data |
|---|---|---|---|
| 54 | 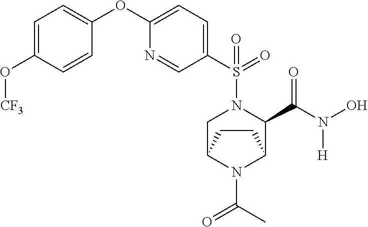 [SMILES: CC(=O)N1[C@@H]2CC[C@H]1[C@H](C(=O)NO)N(S(=O)(=O)c1ccc(Oc3ccc(OC(F)(F)F)cc3)nc1)C2] | (1S,2R,5R)-8-acetyl- N-hydroxy-3-((6-(4- (trifluoromethoxy)- phenoxy)pyridin-3- yl)sulfonyl)-3,8- diazabicyclo[3.2.1]- octane-2- carboxamide | ¹HNMR (400 MHZ, DMSO- d₆): δ 10.75 (brs, 1H), 8.97 (brs, 1H), 8.46-8.44 (m, 1H), 8.16-8.11 (m, 1H), 7.46 (d, J = 8.4 Hz, 2H), 7.38-7.23 (m, 2H), 7.15 (d, J = 8.0 Hz, 1H), 4.80-4.55 (m, 1H), 4.42- 4.31 (m, 1H), 4.14-4.09 (m, 1H), 3.61 -3.35 (m, 2H), 2.05- 1.91 (m, 1H), 1.81 (s, 3H), 1.78-1.40 (m, 3H); MS (ESI): m/z 531.1 [M + H]⁺. |
| 55 | 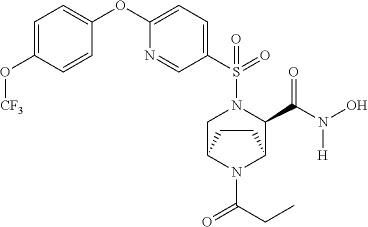 [SMILES: CCC(=O)N1[C@@H]2CC[C@H]1[C@H](C(=O)NO)N(S(=O)(=O)c1ccc(Oc3ccc(OC(F)(F)F)cc3)nc1)C2] | (1S,2R,5R)-N- hydroxy-8- propionyl-3-((6-(4- (trifluoromethoxy)- phenoxy)pyridin-3- yl)sulfonyl)-3,8- diazabicyclo[3.2.1]- octane-2- carboxamide | ¹HNMR (400 MHZ, DMSO- d₆): δ 10.74 (brs, 1H), 8.95 (brs, 1H), 8.49-8.41 (m, 1H), 8.15-8.11 (m, 1H), 7.46 (d, J = 8.8 Hz, 2H), 7.36 (d, J = 9.2 Hz, 2H), 7.25 (d, J = 8.8 Hz, 1H), 4.80-4.58 (m, 1H), 4.46- 4.38 (m, 1H), 4.13-4.08 (m, 1H), 3.70 -3.35 (m, 2H), 2.30- 2.10 (m, 1H), 2.05-1.91 (m, 1H), 1.90-1.40 (m, 4H), 0.95- 0.80 (m, 3H); MS (ESI): m/z 545.1 [M + H]⁺. |
| 56 | 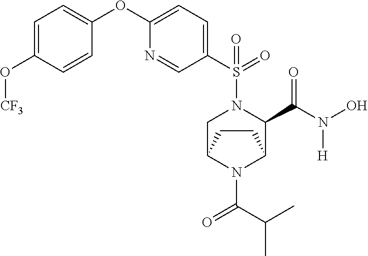 [SMILES: CC(C)C(=O)N1[C@@H]2CC[C@H]1[C@H](C(=O)NO)N(S(=O)(=O)c1ccc(Oc3ccc(OC(F)(F)F)cc3)nc1)C2] | (1S,2R,5R)-N- hydroxy-8- isobutyry1-3-((6-(4- (trifluoro- methoxy)phenoxy)- pyridin-3-yl)- sulfonyl)-3,8- diazabicyclo- [3.2.1]octane-2- carboxamide | ¹HNMR (400 MHZ, DMSO- d₆): δ 10.72 (brs, 1H), 8.80 (brs, 1H), 8.49-8.40 (m, 1H), 8.16-8.09 (m, 1H), 7.46 (d, J = 8.8 Hz, 2H), 7.36 (d, J = 8.8 Hz, 2H), 7.24 (d, J = 8.4 Hz, 1H), 4.80-4.40 (m, 2H), 4.13- 4.09 (m, 1H), 3.65 -3.35 (m, 2H), 2.70-2.60 (m, 1H), 2.05- 1.90 (m, 1H), 1.90-1.40 (m, 3H), 0.99-0.80 (m, 6H); MS (ESI): m/z 559.0 [M + H]⁺. |
| 57 | 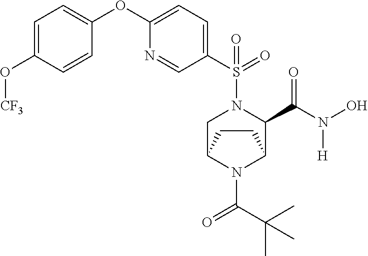 [SMILES: CC(C)(C)C(=O)N1[C@@H]2CC[C@H]1[C@H](C(=O)NO)N(S(=O)(=O)c1ccc(Oc3ccc(OC(F)(F)F)cc3)nc1)C2] | (1S,2R,5R)-N- hydroxy-8-pivaloyl- 3-((6-(4-(trifluoro- methoxy)phenoxy)- pyridin-3-yl)- sulfonyl)-3,8- diazabicyclo- [3.2.1 ]octane-2- carboxamide | ¹HNMR (400 MHZ, DMSO- d₆): δ 10.59 (brs, 1H), 8.76 (brs, 1H), 8.45 (d, J = 2.0 Hz, 1H), 8.11 (dd, J = 2.8, 8.8 Hz, 1H), 7.46 (d, J = 8.4 Hz, 2H), 7.38-7.33 (m, 2H), 7.25 (d, J = 8.8 Hz, 1H), 4.82-4.80 (m, 1H), 4.75-4.69 (m, 1H), 4.08 (d, J = 2.0 Hz, 1H), 3.66-3.63 (m, 1H), 3.48 -3.42 (m, 1H), 1.85-1.70 (m, 2H), 1.65 - 1.49 (m, 2H), 1.09 (s, 9H); MS (ESI): m/z 573.1 [M + H]⁺. |
| 58 | 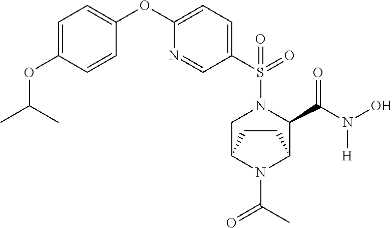 [SMILES: CC(=O)N1[C@@H]2CC[C@H]1[C@H](C(=O)NO)N(S(=O)(=O)c1ccc(Oc3ccc(OC(C)C)cc3)nc1)C2] | (1S,2R,5R)-8-acetyl- N-hydroxy-3-((6-(4- isopropoxyphenoxy)- pyridin-3-yl)- sulfonyl)-3,8- diazabicyclo- [3.2.1]octane-2- carboxamide | ¹HNMR (400 MHZ, DMSO- d₆): δ 10.75 (brs, 1H), 8.95 (brs, 1H), 8.44-8.41 (m, 1H), 8.10-8.05 (m, 1H), 7.12- 7.07 (m, 3H), 6.98-6.96 (m, 2H), 4.80-4.55 (m, 2H), 4.42- 4.38 (m, 1H), 4.13-4.09 (m, 1H), 3.61-3.55 (m, 2H), 2.05- 1.91 (m, 1H), 1.81 (s, 3H), 1.79-1.40 (m, 3H), 1.25 (d, J = 6.0 Hz, 6H); MS (ESI): m/z 505.1 [M + H]⁺ |
| 59 | 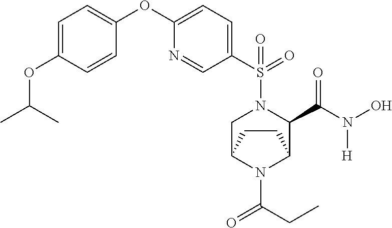 [SMILES: CCC(=O)N1[C@@H]2CC[C@H]1[C@H](C(=O)NO)N(S(=O)(=O)c1ccc(Oc3ccc(OC(C)C)cc3)nc1)C2] | (1S,2R,5R)-N- hydroxy-3-((6-(4- isopropoxyphenoxy) pyridin-3-yl)- sulfonyl)-8- propionyl-3,8-diaza- bicyclo[3.2.1 ]octane- 2-carboxamide | ¹HNMR (400 MHZ, DMSO- d₆): δ 10.73 (brs, 1H), 8.95 (brs, 1H), 8.44-8.41 (m, 1H), 8.10-8.05 (m, 1H), 7.19- 7.07 (m, 3H), 6.98-6.94 (m, 2H), 4.80-4.55 (m, 2H), 4.49- 4.35 (m, 1H), 4.13-4.08 (m, 1H), 3.61-3.38 (m, 2H), 2.29- 1.14 (m, 1H), 2.10-1.90 (m, 1H), 1.79-1.39 (m, 4H), 1.28 (d, J = 6.0 Hz, 6H), 0.94-0.87 (m, 3H); MS (ESI): m/z 519.1 [M + H]⁺. |
| 60 | 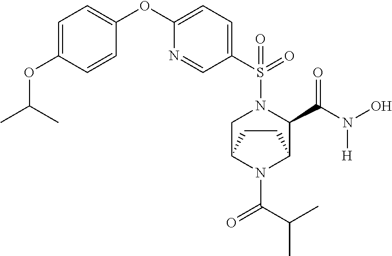 [SMILES: CC(C)Oc1ccc(Oc2ccc(S(=O)(=O)N3C[C@H]4CC[C@@H]([C@@H]3C(=O)NO)N4C(=O)C(C)C)cn2)cc1] | (1S,2R,5R)-N- hydroxy-8- isobutyry1-3-((6-(4- isopropoxy- phenoxy)pyridin-3- yl)sulfonyl)-3,8- diazabicyclo- [3.2.1]octane-2- carboxamide | ¹HNMR (400 MHZ, DMSO- d₆): δ 10.70 (brs, 1H), 8.87 (brs, 1H), 8.46-8.41 (m, 1H), 8.09-8.05 (m, 1H), 7.12- 7.07 (m, 3H), 6.96 (d, J = 9.2 Hz, 2H), 4.80-4.45 (m, 2H), 4.44-4.40 (m, 1H), 4.14- 4.09 (m, 1H), 3.65-3.38 (m, 2H), 2.05-1.90 (m, 1H), 1.89- 1.40 (m, 4H), 1.28 (d, J = 6.0 Hz, 6H), 0.99-0.80 (m, 6H); MS (ESI): m/z 533.1 [M + H]⁺. |
| 61 | 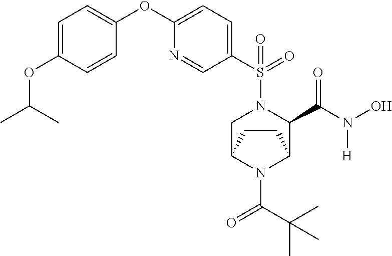 [SMILES: CC(C)Oc1ccc(Oc2ccc(S(=O)(=O)N3C[C@H]4CC[C@@H]([C@@H]3C(=O)NO)N4C(=O)C(C)(C)C)cn2)cc1] | (1S,2R,5R)-N- hydroxy-3-((6-(4- isopropoxyphenoxy)- pyridin-3-yl)- sulfonyl)-8-pivaloyl- 3,8-diazabicyclo- [3.2.1 ]octane-2- carboxamide | ¹HNMR (400 MHZ, DMSO- d₆): δ 10.58 (brs, 1H), 8.76 (brs, 1H), 8.42 (d, J = 2.0 Hz, 1H), 8.07-8.04 (m, 1H), 7.12- 7.07 (m, 3H), 6.98-6.95 (m, 2H), 4.88-4.82 (m, 1H), 4.75- 4.66 (m, 1H), 4.65-4.55 (m, 1H), 4.07 (d, J = 2.4 Hz, 1H), 3.69-3.62 (m, 1H), 3.46- 3.41 (m, 1H), 1.83-1.70 (m, 2H), 1.62-1.49 (m, 2H), 1.28 (d, J = 6.0 Hz, 6H), 1.09 (m, 9H); MS (ESI): m/z 547.3 [M + H]⁺. |
| 62 | 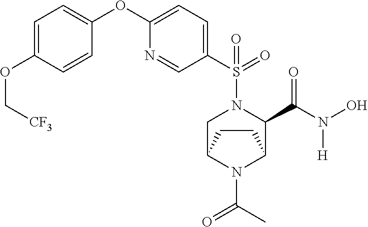 [SMILES: CC(=O)N1[C@@H]2CC[C@H]1[C@H](C(=O)NO)N(S(=O)(=O)c1ccc(Oc3ccc(OCC(F)(F)F)cc3)nc1)C2] | (1S,2R,5R)-8-acetyl- N-hydroxy-3-((6-(4- (2,2,2-trifluoro- ethoxy)phenoxy)- pyridin-3-yl)- sulfonyl)-3,8- diazabicyclo[3.2.1]- octane-2- carboxamide | ¹HNMR (400 MHZ, DMSO- d₆): δ 10.74 (brs, 1H), 8.98 (brs, 1H), 8.45-8.41 (m, 1H), 8.12-8.07 (m, 1H), 7.20- 7.11 (m, 5H), 4.79 (q, J = 8.8 Hz, 2H), 4.70-4.55 (m, 1H), 4.42-4.30 (m, 1H), 4.13- 4.09 (m, 1H), 3.61 -3.35 (m, 2H), 2.05-1.91 (m, 1H), 1.81 (s, 3H), 1.75-1.40 (m, 3H); MS (ESI): m/z 545.2 [M + H]⁺. |
| 63 | 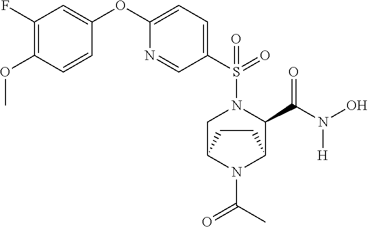 [SMILES: COc1ccc(Oc2ccc(S(=O)(=O)N3C[C@H]4CC[C@@H]([C@@H]3C(=O)NO)N4C(C)=O)cn2)cc1F] | (1S,2R,5R)-8-acetyl- 3-((6-(3-fluoro-4- methoxyphenoxy)- pyridin-3-yl)- sulfonyl)-N- hydroxy-3,8- diazabicyclo[3.2.1]- octane-2- carboxamide | ¹HNMR (400 MHZ, DMSO- d₆): δ 10.75 (brs, 1H), 8.98 (brs, 1H), 8.45-8.42 (m, 1H), 8.12-8.07 (m, 1H), 7.25- 7.14 (m, 3H), 7.03-6.99 (m, 1H), 4.80-4.55 (m, 1H), 4.45- 4.30 (m, 1H), 4.14-4.08 (m, 1H), 3.86 (s, 3H), 3.62-3.35 (m, 2H), 2.05-1.92 (m, 1H), 1.81 (s, 3H), 1.78-1.39 (m, 3H); MS (ESI): m/z 495.2 [M + H]⁺. |
| 64 | 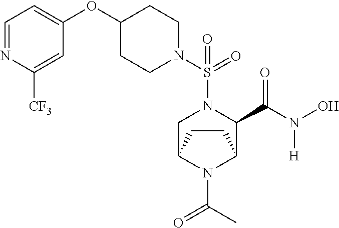 [SMILES: CC(=O)N1[C@@H]2CC[C@H]1[C@H](C(=O)NO)N(S(=O)(=O)N1CCC(Oc3ccnc(C(F)(F)F)c3)CC1)C2] | (1S,2R,5R)-8-acetyl- N-hydroxy-3-((4-((2- (trifluoromethyl)- pyridin-4-yl)oxy)- piperidin-1-yl)- sulfonyl)-3,8- diazabicyclo[3.2.1]- octane-2- carboxamide | ¹H NMR (400 MHZ, DMSO- d₆): δ 10.30 (brs, 1H), 9.03 (brs, 1H), 8.56 (d, J = 5.6 Hz, 1H), 7.47-7.46 (m, 1H), 7.34- 7.31 (m, 1H), 4.90-4.80 (m, 1H), 4.75-4.63 (m, 1H), 4.59- 4.57 (m, 1H), 4.39-4.29 (m, 1H), 3.94-3.83 (m, 1H), 3.72- 3.60 (m, 1H), 3.41-3.35 (m, 2H), 3.14-3.05 (m, 2H), 2.10- 1.85 (m, 7H), 1.81-1.60 (m, 4H); MS (ESI): m/z 522.2 [M + H]⁺. |

### Example 65

### Synthesis of ethyl (1S,2R,5R)-3-((6-(4-fluorophenoxy)pyridin-3-yl)sulfonyl)-2-(hydroxycarbamoyl)-3,8-diazabicyclo[3.2.1]octane-8-carboxylate

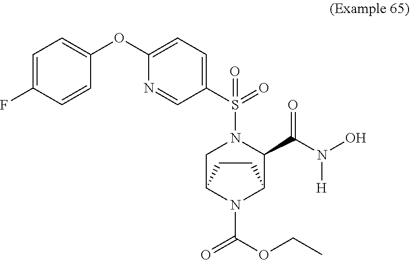 [SMILES: CCOC(=O)N1[C@@H]2CC[C@H]1[C@H](C(=O)NO)N(S(=O)(=O)c1ccc(Oc3ccc(F)cc3)nc1)C2]

### Step 1

### Synthesis of 8-ethyl 2-methyl (1S,2S,5R)-3-((6-(4-fluorophenoxy)pyridin-3-yl)sulfonyl)-3,8-diazabicyclo[3.2.1]octane-2,8-dicarboxylate

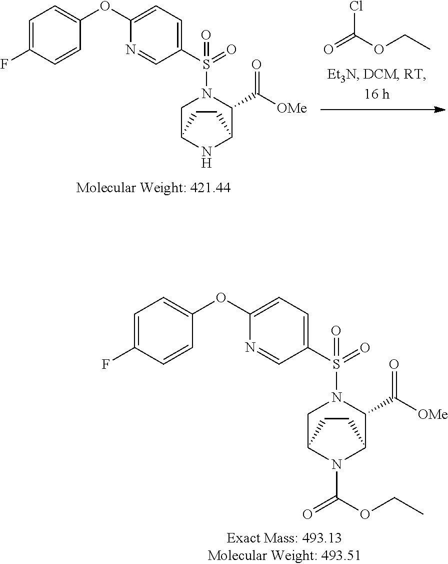

To a stirred solution of methyl (1S,2S,5R)-3-((6-(4-fluorophenoxy)pyridin-3-yl)sulfonyl)-3,8-diazabicyclo[3.2.1]octane-2-carboxylate (150 mg, 0.35 mmol) in DCM (5 mL) at 0-10° C. was added Et₃N (0.15 mL, 1.05 mmol) and stirred for 10 min Ethyl chloroformate (0.08 mL, 0.7 mmol) was added and the reaction was stirred at RT for 16 h while being monitored by TLC. The reaction was quenched with ice cold water (5 mL) and extracted with DCM (2×20 mL). The organic layer was separated, dried over anhydrous Na₂SO₄, filtered and concentrated under reduced pressure to obtain crude product. The crude product was purified by silica gel column chromatography (SiO₂, 60-120 mesh, 15-25% EtOAc in hexanes) to afford 8-ethyl 2-methyl (1S,2S,5R)-3-((6-(4-fluorophenoxy)pyridin-3-yl)sulfonyl)-3,8-diazabicyclo[3.2.1]octane-2,8-dicarboxylate (140 mg, 0.28 mmol, 80% yield) as a light yellow syrup. MS (ESI): 516.0 [M+23]⁺; ¹H NMR (400 MHz, CDCl₃): 8.62 (d, J=2.4 Hz, 1H), 8.22-8.19 (m, 1H), 7.16-7.10 (m, 4H), 7.05 (d, J=8.8 Hz, 1H), 4.41-4.34 (m, 2H), 4.12 (q, J=7.2 Hz, 2H), 3.96-3.88 (m, 1H), 3.82 (s, 3H), 3.20 (dd, J=2.0, 11.2 Hz, 1H), 3.02 (dd, J=2.8, 11.2 Hz, 1H), 2.54-2.47 (m, 1H), 2.04-1.92 (m, 1H), 1.80-1.71 (m, 2H), 1.23 (t, J=7.2 Hz, 3H).

### Step 2

### Synthesis of (1S,2R,5R)-8-(ethoxycarbonyl)-3-((6-(4-fluorophenoxy)pyridin-3-yl)sulfonyl)-3,8-diazabicyclo[3.2.1]octane-2-carboxylic acid

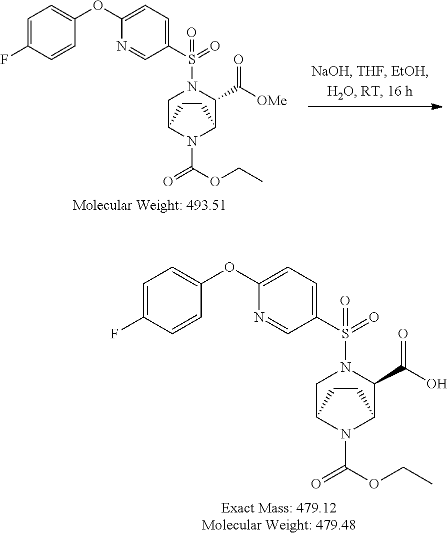 [SMILES: CCOC(=O)N1[C@@H]2CC[C@H]1[C@@H](C(=O)OC)N(S(=O)(=O)c1ccc(Oc3ccc(F)cc3)nc1)C2 ; CCOC(=O)N1[C@@H]2CC[C@H]1[C@H](C(=O)O)N(S(=O)(=O)c1ccc(Oc3ccc(F)cc3)nc1)C2]

Similar procedure was followed as described for Step 4 of Example 1, to obtain (1S,2R,5R)-8-(ethoxycarbonyl)-3-((6-(4-fluorophenoxy)pyridin-3-yl)sulfonyl)-3,8-diazabicyclo[3.2.1]octane-2-carboxylic acid (100 mg, 0.21 mmol, 79% yield) as a light brown gum. This was used as such for the next step without further purification. MS (ESI): m/z 478.1 [M−H]⁻.

### Step 3

### Synthesis of ethyl (1S,2R,5R)-3-((6-(4-fluorophenoxy)pyridin-3-yl)sulfonyl)-2-(((tetrahydro-2H-pyran-2-yl)oxy)carbamoyl)-3,8-diazabicyclo[3.2.1]octane-8-carboxylate

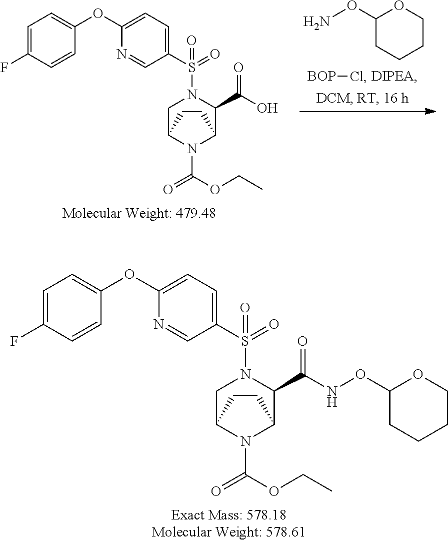

Similar procedure was followed as described for Step 3 of Example 10, to obtain ethyl (1S,2R,5R)-3-((6-(4-fluorophenoxy)pyridin-3-yl)sulfonyl)-2-(((tetrahydro-2H-pyran-2-yl)oxy)carbamoyl)-3,8-diazabicyclo[3.2.1]octane-8-carboxylate (90 mg, 0.15 mmol) as a yellow gum. This was used as such for the next step without further purification. MS (ESI): m/z 577.1 [M−H]⁻.

### Step 4

### Synthesis of ethyl (1S,2R,5R)-3-((6-(4-fluorophenoxy)pyridin-3-yl)sulfonyl)-2-(hydroxycarbamoyl)-3,8-diazabicyclo[3.2.1]octane-8-carboxylate (Example 65)

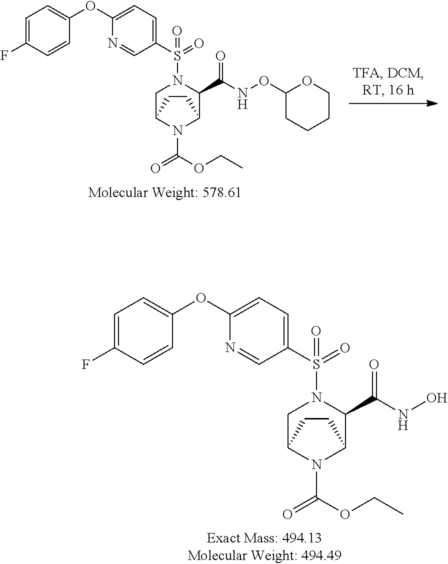 [SMILES: CCOC(=O)N1[C@@H]2CC[C@H]1[C@H](C(=O)NOC1CCCCO1)N(S(=O)(=O)c1ccc(Oc3ccc(F)cc3)nc1)C2 ; CCOC(=O)N1[C@@H]2CC[C@H]1[C@H](C(=O)NO)N(S(=O)(=O)c1ccc(Oc3ccc(F)cc3)nc1)C2]

Similar procedure was followed as described for Step 6 of Example 1 and purification was done by RP prep HPLC (Table 2, Method 4) to obtain ethyl (1S,2R,5R)-3-((6-(4-fluorophenoxy)pyridin-3-yl)sulfonyl)-2-(hydroxycarbamoyl)-3,8-diazabicyclo[3.2.1]octane-8-carboxylate (Example 65) (11 mg, 0.02 mmol, 14% yield for 2 steps) as an off-white solid. MS (ESI): m/z 495.2 [M+H]⁺; #H NMR (400 MHz, DMSO-d₆): δ 10.62 (brs, 1H), 8.80 (brs, 1H), 8.43 (d, J=2.4 Hz, 1H), 8.09 (dd, J=2.8, 8.6 Hz, 1H), 7.35-7.27 (m, 4H), 7.19 (d, J=8.8 Hz, 1H), 4.70-4.36 (m, 1H), 4.30-4.20 (m, 1H), 4.11-4.00 (m, 1H), 3.99-3.81 (m, 2H), 3.60-3.50 (m, 1H), 3.45-3.35 (m, 1H), 2.00-1.71 (m, 2H), 1.70-1.42 (m, 2H), 1.12 (t, J=6.8 Hz, 3H).

Similarly, below examples were prepared by following the same or analogous process as described in Example 65.

TABLE 11

| Ex. | Structure | Name | Analytical data |
|---|---|---|---|
| 66 | 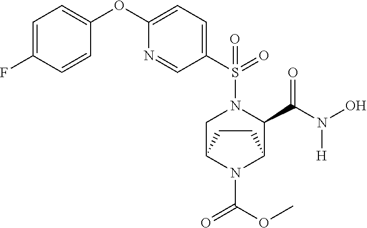 [SMILES: COC(=O)N1[C@@H]2CC[C@H]1[C@H](C(=O)NO)N(S(=O)(=O)c1ccc(Oc3ccc(F)cc3)nc1)C2] | methyl (1S,2R,5R)- 3-((6-(4-fluoro- phenoxy)pyridin-3- yl)sulfonyl)-2- (hydroxycarbamoyl)- 3,8-diazabicyclo- [3.2.1]octane-8- carboxylate | ¹H NMR (400 MHz, DMSO- d₆): δ 10.60 (brs, 1H), 8.82 (brs, 1H), 8.43 (d, J = 2.4 Hz, 1H), 8.10 (dd, J = 2.4, 8.8 Hz, 1H), 7.31-7.23 (m, 4H), 7.19 (d, J = 8.8 Hz, 1H), 4.61- 4.31 (m, 1H), 4.30-4.20 (m, 1H), 4.15-3.99 (m, 1H), 3.60- 3.35 (m, 5H), 1.99-1.70 (m, 2H), 1.69-1.40 (m, 2H); MS (ESI): m/z 479.1 [M − H]⁻. |
| 67 | 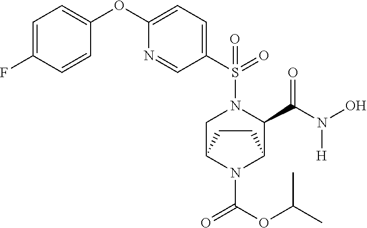 [SMILES: CC(C)OC(=O)N1[C@@H]2CC[C@H]1[C@H](C(=O)NO)N(S(=O)(=O)c1ccc(Oc3ccc(F)cc3)nc1)C2] | isopropyl (1S,2R,5R)-3-((6-(4- fluorophenoxy)- pyridin-3-yl)- sulfonyl)-2- (hydroxycarbamoyl)- 3,8-diazabicyclo- [3.2.1]octane-8- carboxylate | ¹H NMR (400 MHz, DMSO- d₆): δ 10.60 (brs, 1H), 8.78 (brs, 1H), 8.43 (d, J = 2.4 Hz, 1H), 8.09 (dd, J = 2.4, 8.8 Hz, 1H), 7.31-7.21 (m, 4H), 7.18 (d, J = 8.8 Hz, 1H), 4.71- 4.54 (m, 1H), 4.49-4.35 (m, 1H), 4.29-4.20 (m, 1H), 4.10- 3.99 (m, 1H), 3.60-3.50 (m, 1H), 3.45-3.35 (m, 1H), 2.00-1.70 (m, 2H), 1.69- 1.42 (m, 2H), 1.12 (d, J = 6.4 Hz, 6H); MS (ESI): m/z 509.1 [M + H]⁺. |

### Example 68

### Synthesis of (1S,2R,5R)-3-((6-(4-fluorophenoxy)pyridin-3-yl)sulfonyl)-N-hydroxy-8-(morpholine-4-carbonyl)-3,8-diazabicyclo[3.2.1]octane-2-carboxamide

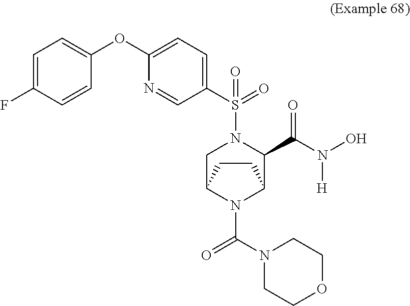 [SMILES: O=C(NO)[C@H]1[C@@H]2CC[C@H](CN1S(=O)(=O)c1ccc(Oc3ccc(F)cc3)nc1)N2C(=O)N1CCOCC1]

### Step 1

### Synthesis of methyl (1S,2S,5R)-3-((6-(4-fluorophenoxy)pyridin-3-yl)sulfonyl)-8-(morpholine-4-carbonyl)-3,8-diazabicyclo[3.2.1]octane-2-carboxylate

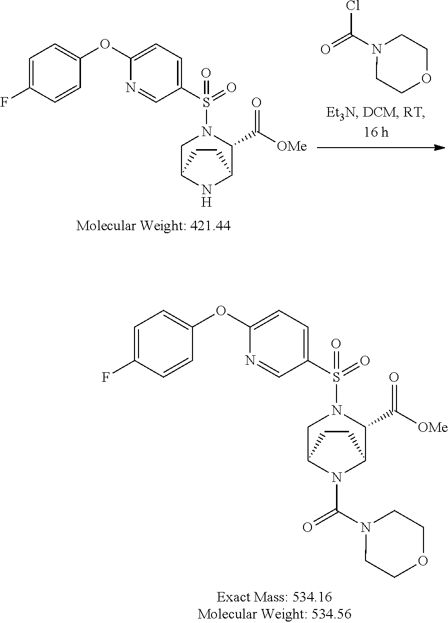 [SMILES: COC(=O)[C@@H]1[C@@H]2CC[C@H](CN1S(=O)(=O)c1ccc(Oc3ccc(F)cc3)nc1)N2 ; O=C(Cl)N1CCOCC1 ; COC(=O)[C@@H]1[C@@H]2CC[C@H](CN1S(=O)(=O)c1ccc(Oc3ccc(F)cc3)nc1)N2C(=O)N1CCOCC1]

To a stirred solution of methyl (1S,2S,5R)-3-((6-(4-fluorophenoxy)pyridin-3-yl)sulfonyl)-3,8-diazabicyclo[3.2.1]octane-2-carboxylate (120 mg, 0.28 mmol) in DCM (5 mL) at 0-10° C. was added Et₃N (0.12 mL, 0.85 mmol) and stirred for 10 min. Morpholine-4-carbonyl chloride (0.07 mL, 0.68 mmol) was added and the reaction was stirred at RT for 16 h while being monitored by TLC. The reaction was quenched with ice cold water (5 mL) and extracted with DCM (2×15 mL). The organic layer was separated, dried over anhydrous Na₂SO₄, filtered and concentrated under reduced pressure to afford methyl (1S,2S,5R)-3-((6-(4-fluorophenoxy)pyridin-3-yl)sulfonyl)-8-(morpholine-4-carbonyl)-3,8-diazabicyclo[3.2.1]octane-2-carboxylate (150 mg, 0.28 mmol, quantitative) as a light yellow syrup. This was used as such for the next step without further purification. MS (ESI): m/z 535.0 [M+H]⁺.

### Step 2

### Synthesis of (1S,2R,5R)-3-((6-(4-fluorophenoxy)pyridin-3-yl)sulfonyl)-8-(morpholine-4-carbonyl)-3,8-diazabicyclo[3.2.1]octane-2-carboxylic acid

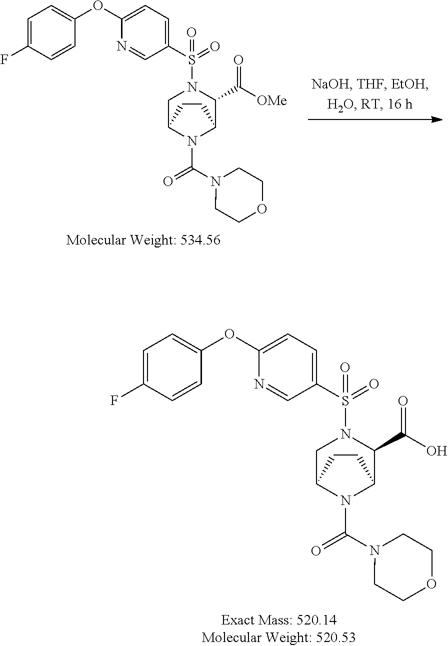 [SMILES: COC(=O)[C@@H]1[C@@H]2CC[C@H](CN1S(=O)(=O)c1ccc(Oc3ccc(F)cc3)nc1)N2C(=O)N1CCOCC1 ; O=C(O)[C@H]1[C@@H]2CC[C@H](CN1S(=O)(=O)c1ccc(Oc3ccc(F)cc3)nc1)N2C(=O)N1CCOCC1]

Similar procedure was followed as described for Step 4 of Example 1, to obtain (1S,2R,5R)-3-((6-(4-fluorophenoxy)pyridin-3-yl)sulfonyl)-8-(morpholine-4-carbonyl)-3,8-diazabicyclo[3.2.1]octane-2-carboxylic acid (140 mg, 0.27 mmol) as a light yellow sticky gum. This was used as such for the next step without further purification. MS (ESI): m/z 521.2 [M+H]⁺.

### Step 3

### Synthesis of (1S,2R,5R)-3-((6-(4-fluorophenoxy)pyridin-3-yl)sulfonyl)-8-(morpholine-4-carbonyl)-N-((tetrahydro-2H-pyran-2-yl)oxy)-3,8-diazabicyclo[3.2.1]octane-2-carboxamide

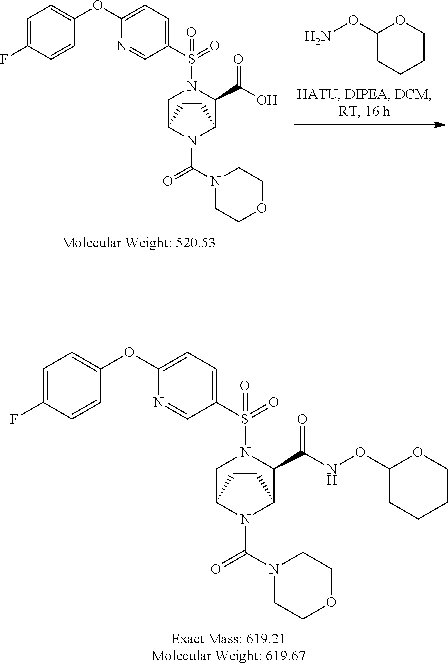 [SMILES: O=C(O)[C@H]1[C@@H]2CC[C@H](CN1S(=O)(=O)c1ccc(Oc3ccc(F)cc3)nc1)N2C(=O)N1CCOCC1 ; NOC1CCCCO1 ; O=C(NOC1CCCCO1)[C@H]1[C@@H]2CC[C@H](CN1S(=O)(=O)c1ccc(Oc3ccc(F)cc3)nc1)N2C(=O)N1CCOCC1]

Similar procedure was followed as described for Step 5 of Example 1, to obtain (1S,2R,5R)-3-((6-(4-fluorophenoxy)pyridin-3-yl)sulfonyl)-8-(morpholine-4-carbonyl)-N-((tetrahydro-2H-pyran-2-yl)oxy)-3,8-diazabicyclo[3.2.1]octane-2-carboxamide (100 mg, 0.16 mmol) as a yellow color sticky compound. This was used as such for the next step without further purification. MS (ESI): m/z 620.2 [M+H]⁺.

### Step 4

### Synthesis of (1S,2R,5R)-3-((6-(4-fluorophenoxy)pyridin-3-yl)sulfonyl)-N-hydroxy-8-(morpholine-4-carbonyl)-3,8-diazabicyclo[3.2.1]octane-2-carboxamide (Example 68)

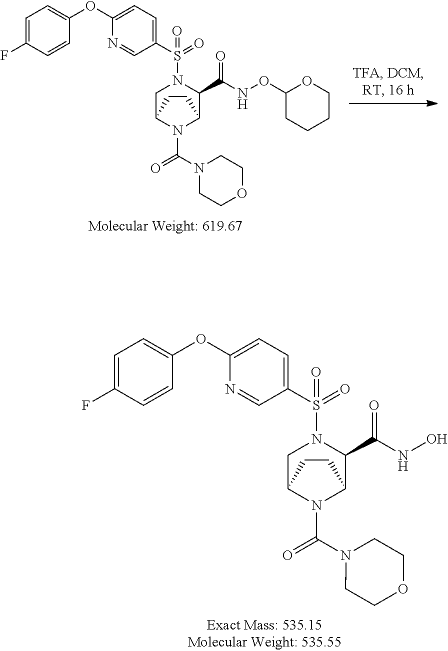 [SMILES: O=C(NOC1CCCCO1)[C@H]1[C@@H]2CC[C@H](CN1S(=O)(=O)c1ccc(Oc3ccc(F)cc3)nc1)N2C(=O)N1CCOCC1 ; O=C(NO)[C@H]1[C@@H]2CC[C@H](CN1S(=O)(=O)c1ccc(Oc3ccc(F)cc3)nc1)N2C(=O)N1CCOCC1]

Similar procedure was followed as described for Step 6 of Example 1 and purification was done by RP prep HPLC (Table 2, Method 3) to obtain (1S,2R,5R)-3-((6-(4-fluorophenoxy)pyridin-3-yl)sulfonyl)-N-hydroxy-8-(morpholine-4-carbonyl)-3, 8-diazabicyclo[3.2.1]octane-2-carboxamide (Example 68) (16 mg, 0.03 mmol, 18% yield for 2 steps) as an off-white solid. MS (ESI): m/z 536.2 [M+H]⁺; ¹H NMR (400 MHz, DMSO-d₆): δ 10.54 (brs, 1H), 8.84 (brs, 1H), 8.45 (d, J=2.4 Hz, 1H), 8.14-8.09 (m, 1H), 7.31-7.24 (m, 4H), 7.20 (d, J=8.8 Hz, 1H), 4.62-4.27 (m, 1H), 4.15 (d, J=2.4 Hz, 1H), 4.20-3.97 (m, 1H), 3.67-3.60 (m, 1H), 3.58-3.46 (m, 4H), 3.45-3.35 (m, 1H), 3.27-3.09 (m, 4H), 1.89-1.72 (m, 1H), 1.71-1.55 (m, 1H), 1.54-1.39 (m, 2H).

Similarly, below examples were prepared by following the same or analogous process as described in Example 68.

TABLE 12

| Ex. | Structure | Name | Analytical data |
|---|---|---|---|
| 69 | 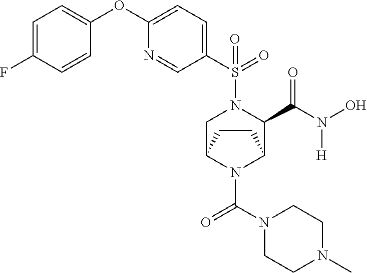 [SMILES: CN1CCN(C(=O)N2[C@@H]3CC[C@H]2[C@H](C(=O)NO)N(S(=O)(=O)c2ccc(Oc4ccc(F)cc4)nc2)C3)CC1] | (1S,2R,5R)-3-((6-(4- fluorophenoxy)pyridin- 3-yl)sulfonyl)-N- hydroxy-8-(4-methyl- piperazine-1-carbonyl)- 3,8-diazabicyclo- [3.2.1]octane-2- carboxamide | ¹H NMR (400 MHz, DMSO- d₆): δ 10.57 (brs, 1H), 8.84 (brs, 1H), 8.45 (d, J = 2.4 Hz, 1H), 8.15-8.09 (m, 1H), 7.32- 7.24 (m, 4H), 7.20 (d, J = 8.8 Hz, 1H), 4.65-4.23 (m, 1H), 4.15 (d, J = 2.4 Hz, 1H), 3.99-3.91 (m, 1H), 3.66- 3.60 (m, 1H), 3.41-3.35 (m, 1H), 3.25-3.10 (m, 4H), 2.28- 2.18 (m, 4H), 2.15 (s, 3H), 1.83-1.71 (m, 1H), 1.69- 1.52 (m, 1H), 1.51-1.38 (m, 2H); MS (ESI): m/z 549.2 [M + H]⁺. |
| 70 | 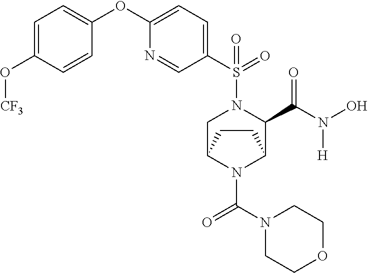 [SMILES: O=C(NO)[C@H]1[C@@H]2CC[C@H](CN1S(=O)(=O)c1ccc(Oc3ccc(OC(F)(F)F)cc3)nc1)N2C(=O)N1CCOCC1] | (1S,2R,5R)-N- hydroxy-8- (morpholine-4- carbonyl)-3-((6-(4- (trifluoromethoxy)- phenoxy)pyridin-3- yl)sulfonyl)-3,8- diazabicyclo[3.2.1]- octane-2-carboxamide | ¹HNMR (400 MHz, DMSO- d₆): δ 10.58 (brs, 1H), 8.85 (brs, 1H), 8.47-8.44 (m, 1H), 8.16-8.11 (m, 1H), 7.47 (d, J = 8.4 Hz, 2H), 7.38-7.33 (m, 2H), 7.15 (d, J = 8.0 Hz, 1H), 4.62-4.28 (m, 1H), 4.16- 4.15 (m, 1H), 4.05-3.95 (m, 1H), 3.70-3.60 (m, 1H), 3.58- 3.45 (m, 4H), 3.45-3.35 (m, 1H), 3.26-3.09 (m, 4H), 1.88- 1.75 (m, 1H), 1.70-1.55 (m, 1H), 1.54-1.39 (m, 2H); MS (ESI): m/z 602.2 [M + H]⁺. |
| 71 | 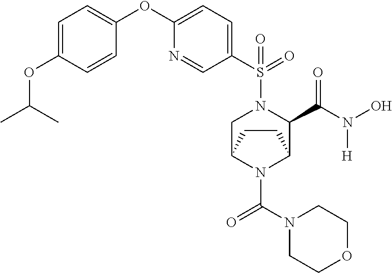 [SMILES: CC(C)Oc1ccc(Oc2ccc(S(=O)(=O)N3C[C@H]4CC[C@@H]([C@@H]3C(=O)NO)N4C(=O)N3CCOCC3)cn2)cc1] | (1S,2R,5R)-N- hydroxy-3-((6-(4- isopropoxyphenoxy)- pyridin-3-yl)sulfonyl)- 8-(morpholine-4- carbonyl)-3,8- diazabicyclo[3.2.1]- octane-2-carboxamide | ¹HNMR (400 MHz, DMSO- d₆): δ 10.57 (brs, 1H), 8.85 (brs, 1H), 8.44 (d, J = 2.4 Hz, 1H), 8.08 (dd, J = 2.4, 8.8 Hz, 1H), 7.13-7.07 (m, 3H), 6.99- 6.95 (m, 2H), 4.63-4.52 (m, 1H), 4.30-4.27 (m, 1H), 4.15 (d, J = 2.4 Hz, 1H), 4.02- 3.96 (m, 1H), 3.69-3.59 (m, 1H), 3.56-3.35 (m, 5H), 3.28-3.09 (m, 4H), 1.86- 1.75 (m, 1H), 1.72-1.55 (m, 1H), 1.52-1.35 (m, 2H), 1.28 (d, J = 6.0 Hz, 6H); MS (ESI): m/z 576.3 [M + H]⁺. |
| 72 | 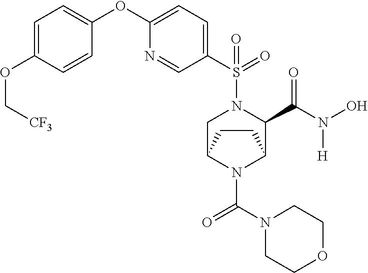 [SMILES: O=C(NO)[C@H]1[C@@H]2CC[C@H](CN1S(=O)(=O)c1ccc(Oc3ccc(OCC(F)(F)F)cc3)nc1)N2C(=O)N1CCOCC1] | (1S,2R,5R)-N- hydroxy-8- (morpholine-4- carbonyl)-3-((6-(4- (2,2,2-trifluoroethoxy)- phenoxy)pyridin-3- yl)sulfonyl)-3,8- diazabicyclo[3.2.1]- octane-2-carboxamide | ¹HNMR (400 MHz, DMSO- d₆): δ 10.58 (brs, 1H), 8.84 (brs, 1H), 8.44 (d, J = 2.4 Hz, 1H), 8.10 (dd, J = 2.4, 8.8 Hz, 1H), 7.20-7.06 (m, 5H), 4.77 (q, J = 8.8 Hz, 2H), 4.62- 4.27 (m, 1H), 4.15 (d, J = 2.4 Hz, 1H), 4.04-3.96 (m, 1H), 3.64-3.60 (m, 1H), 3.58- 3.35 (m, 4H), 3.25-3.10 (m, 5H), 1.83-1.72 (m, 1H), 1.52- 1.39 (m, 2H); MS (ESI): m/z 616.1 [M + H]⁺. |
| 73 | 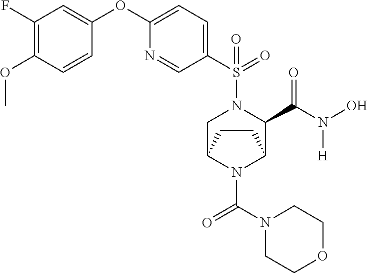 [SMILES: COc1ccc(Oc2ccc(S(=O)(=O)N3C[C@H]4CC[C@@H]([C@@H]3C(=O)NO)N4C(=O)N3CCOCC3)cn2)cc1F] | (1S,2R,5R)-3-((6-(3- fluoro-4-methoxy- phenoxy)pyridin-3-yl)- sulfonyl)-N-hydroxy-8- (morpholine-4- carbonyl)-3,8- diazabicyclo[3.2.1]- octane-2-carboxamide | ¹HNMR (400 MHz, DMSO- d₆): δ 10.57 (brs, 1H), 8.85 (brs, 1H), 8.46-8.43 (m, 1H), 8.12-8.09 (m, 1H), 7.25- 7.15 (m, 3H), 7.03-6.99 (m, 1H), 4.80-4.27 (m, 1H), 4.17- 4.14 (m, 1H), 4.03-3.97 (m, 1H), 3.86 (s, 3H), 3.64- 3.61 (m, 1H), 3.58-3.45 (m, 4H), 3.46-3.36 (m, 1H), 3.27- 3.08 (m, 4H), 1.85-1.75 (m, 1H), 1.74-1.52 (m, 1H), 1.52-1.39 (m, 2H); MS (ESI): m/z 566.3 [M + H]⁺. |

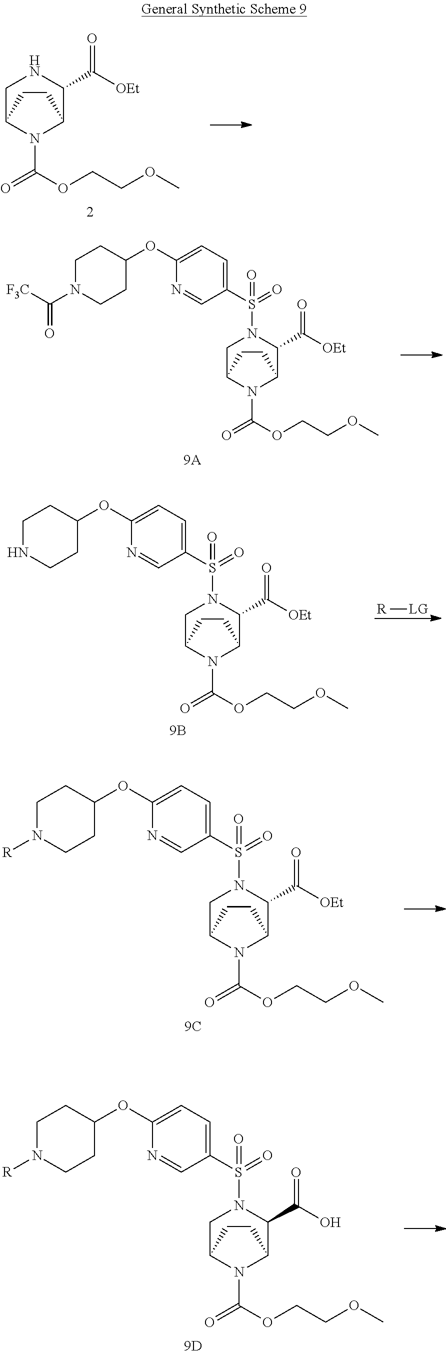

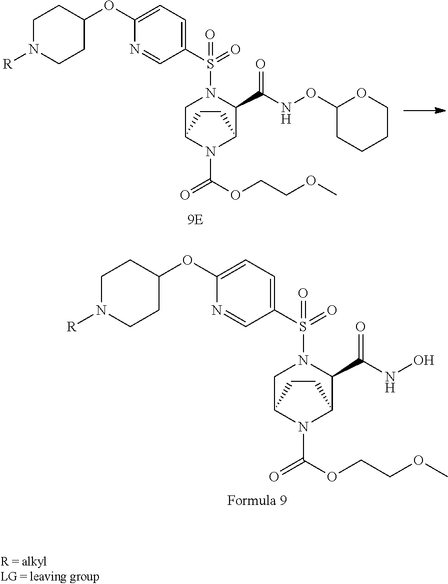

### Example 74

### Synthesis of 2-methoxyethyl (1S,2R,5R)-3-((6-((1-ethylpiperidin-4-yl)oxy)pyridin-3-yl)sulfonyl)-2-(hydroxycarbamoyl)-3,8-diazabicyclo[3.2.1]octane-8-carboxylate

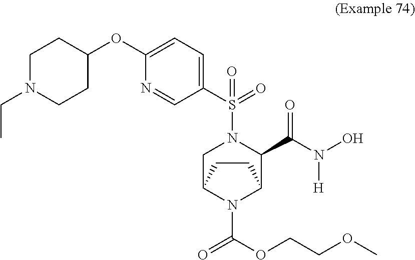 [SMILES: CCN1CCC(Oc2ccc(S(=O)(=O)N3C[C@H]4CC[C@@H]([C@@H]3C(=O)NO)N4C(=O)OCCOC)cn2)CC1]

### Step 1

### Synthesis of 2-ethyl 8-(2-methoxyethyl) (1S,2S,5R)-3-((6-((1-(2,2,2-trifluoroacetyl)piperidin-4-yl)oxy)pyridin-3-yl)sulfonyl)-3,8-diazabicyclo[3.2.1]octane-2,8-dicarboxylate

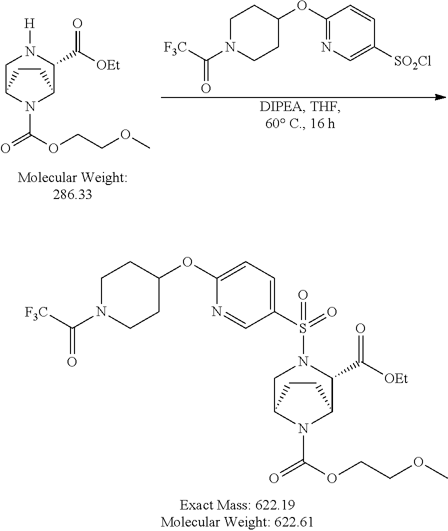 [SMILES: CCOC(=O)[C@H]1NC[C@H]2CC[C@@H]1N2C(=O)OCCOC ; O=C(N1CCC(Oc2ccc(S(=O)(=O)Cl)cn2)CC1)C(F)(F)F ; CCOC(=O)[C@@H]1[C@@H]2CC[C@H](CN1S(=O)(=O)c1ccc(OC3CCN(C(=O)C(F)(F)F)CC3)nc1)N2C(=O)OCCOC]

To a stirring solution of 2-ethyl 8-(2-methoxyethyl) (1S,2S,5R)-3,8-diazabicyclo[3.2.1]octane-2,8-dicarboxylate (1 g, 3.49 mmol) in THF (10 mL) under nitrogen atmosphere at 0-10° C. was added DIPEA (2.5 mL, 13.9 mmol) and stirred for 20 min. 6-((1-(2,2,2-Trifluoroacetyl)piperidin-4-yl)oxy)pyridine-3-sulfonyl chloride (1.95 g, 5.23 mmol) was dissolved in THE (10 mL) and added to the reaction. The reaction was stirred at 60° C. for 16 h while being monitored by TLC. The reaction was cooled to RT, diluted with water (15 mL) and extracted with ethyl acetate (2×30 mL). The combined organic layer was dried over Na₂SO₄, filtered and concentrated under reduced pressure to afford 2-ethyl 8-(2-methoxyethyl) (1S,2S,5R)-3-((6-((1-(2,2,2-trifluoroacetyl)piperidin-4-yl)oxy)pyridin-3-yl)sulfonyl)-3, 8-diazabicyclo[3.2.1]octane-2,8-dicarboxylate (2 g, 3.21 mmol, 91% yield) as a light yellow gum. This was used as such for the next step without further purification. MS (ESI): m/z 623.3 [M+H]⁺.

### Step 2

### Synthesis of 2-ethyl 8-(2-methoxyethyl) (1S,2S,5R)-3-((6-(piperidin-4-yloxy)pyridin-3-yl)sulfonyl)-3,8-diazabicyclo[3.2.1]octane-2,8-dicarboxylate

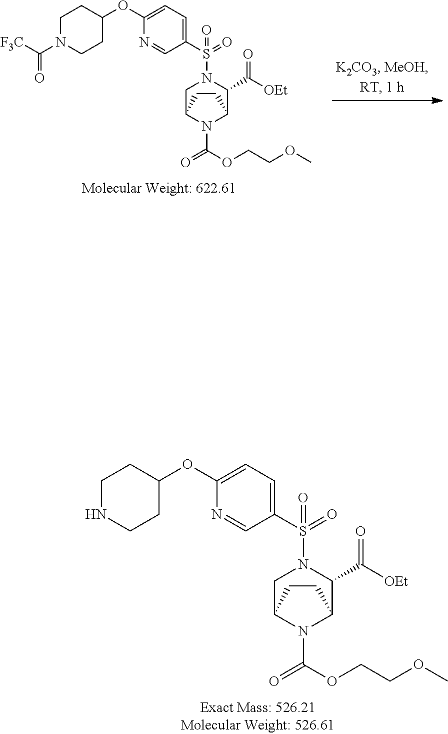 [SMILES: CCOC(=O)[C@@H]1[C@@H]2CC[C@H](CN1S(=O)(=O)c1ccc(OC3CCN(C(=O)C(F)(F)F)CC3)nc1)N2C(=O)OCCOC ; CCOC(=O)[C@@H]1[C@@H]2CC[C@H](CN1S(=O)(=O)c1ccc(OC3CCNCC3)nc1)N2C(=O)OCCOC]

To a stirred solution of 2-ethyl 8-(2-methoxyethyl) (1S,2S,5R)-3-((6-((1-(2,2,2-trifluoroacetyl)piperidin-4-yl)oxy)pyridin-3-yl)sulfonyl)-3,8-diazabicyclo[3.2.1]octane-2,8-dicarboxylate (500 mg, 0.8 mmol) in MeOH (5 mL) at 0-10° C. was added K₂CO₃ (221 mg, 1.6 mmol) and stirred at room temperature for 1 h. The reaction was monitored by TLC. The volatiles were evaporated to afford semi pure 2-ethyl 8-(2-methoxyethyl) (1S,2S,5R)-3-((6-(piperidin-4-yloxy)pyridin-3-yl)sulfonyl)-3,8-diazabicyclo[3.2.1]octane-2,8-dicarboxylate (420 mg, 0.79 mmol, quantitative) as white gum. This was used for next step without further purification. MS (ESI): m/z 527.3 [M+H]⁺.

### Step 3

### Synthesis of 2-ethyl 8-(2-methoxyethyl) (1S,2S,5R)-3-((6-((1-ethylpiperidin-4-yl)oxy)pyridin-3-yl)sulfonyl)-3,8-diazabicyclo[3.2.1]octane-2,8-dicarboxylate

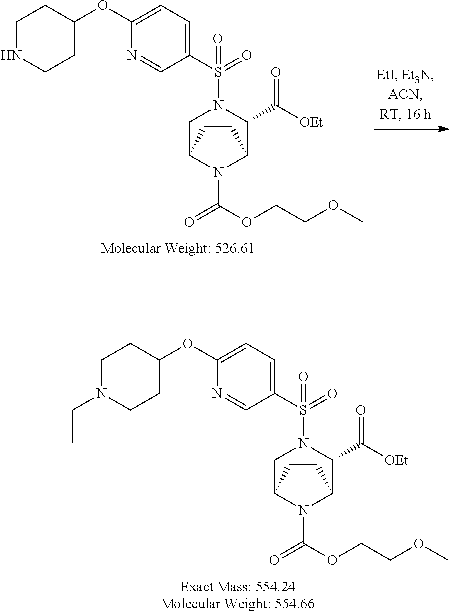 [SMILES: CCOC(=O)[C@@H]1[C@@H]2CC[C@H](CN1S(=O)(=O)c1ccc(OC3CCNCC3)nc1)N2C(=O)OCCOC ; CCOC(=O)[C@@H]1[C@@H]2CC[C@H](CN1S(=O)(=O)c1ccc(OC3CCN(CC)CC3)nc1)N2C(=O)OCCOC]

To a stirred solution of 2-ethyl 8-(2-methoxyethyl) (1S,2S,5R)-3-((6-(piperidin-4-yloxy)pyridin-3-yl)sulfonyl)-3,8-diazabicyclo[3.2.1]octane-2,8-dicarboxylate (420 mg, 0.79 mmol) in ACN (5 mL) at 0-10° C. was added Et₃N (0.46 mL, 3.19 mmol) and ethyl iodide (0.08 mL, 1.04 mmol). The reaction was stirred at RT for 16 h while being monitored by TLC. The reaction was diluted with water (5 mL) and extracted with ethyl acetate (2×20 mL). The combined organic layer was dried over anhydrous Na₂SO₄, filtered and concentrated under reduced pressure to afford 2-ethyl 8-(2-methoxyethyl) (1S,2S,5R)-3-((6-((1-ethylpiperidin-4-yl)oxy)pyridin-3-yl)sulfonyl)-3,8-diazabicyclo[3.2.1]octane-2,8-dicarboxylate (400 mg, 0.72 mmol, 91% yield) as a light yellow syrup. This was used for next step without further purification. MS (ESI): m/z 555.3 [M+H]⁺.

### Step 4

### Synthesis of (1S,2R,5R)-3-((6-((1-ethylpiperidin-4-yl)oxy)pyridin-3-yl)sulfonyl)-8-((2-methoxyethoxy)carbonyl)-3,8-diazabicyclo[3.2.1]octane-2-carboxylic acid

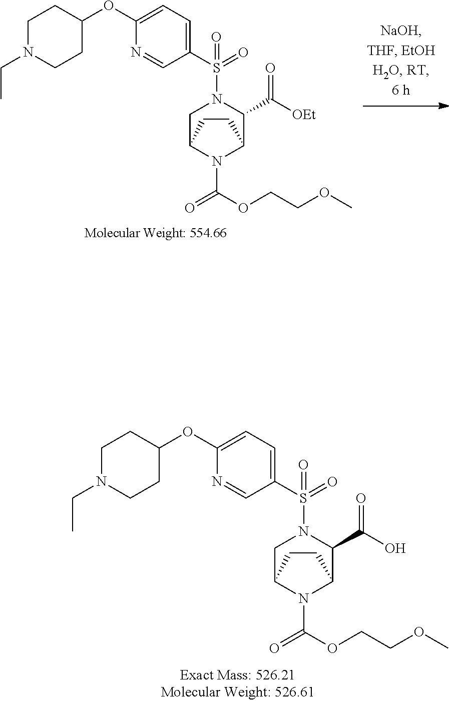 [SMILES: CCOC(=O)[C@@H]1[C@@H]2CC[C@H](CN1S(=O)(=O)c1ccc(OC3CCN(CC)CC3)nc1)N2C(=O)OCCOC ; CCN1CCC(Oc2ccc(S(=O)(=O)N3C[C@H]4CC[C@@H]([C@@H]3C(=O)O)N4C(=O)OCCOC)cn2)CC1]

Similar procedure was followed as described for Step 4 of Example 1, to obtain (1S,2R,5R)-3-((6-((1-ethylpiperidin-4-yl)oxy)pyridin-3-yl)sulfonyl)-8-((2-methoxyethoxy)carbonyl)-3,8-diazabicyclo[3.2.1]octane-2-carboxylic acid (230 mg, 0.43 mmol, 60% yield) as a brown gum. This was used as such for the next step without further purification. MS (ESI): m/z 527.3 [M+H]⁺.

### Step 5

### Synthesis of 2-methoxyethyl (1S,2R,5R)-3-((6-((1-ethylpiperidin-4-yl)oxy)pyridin-3-yl)sulfonyl)-2-(((tetrahydro-2H-pyran-2-yl)oxy)carbamoyl)-3,8-diazabicyclo[3.2.1]octane-8-carboxylate

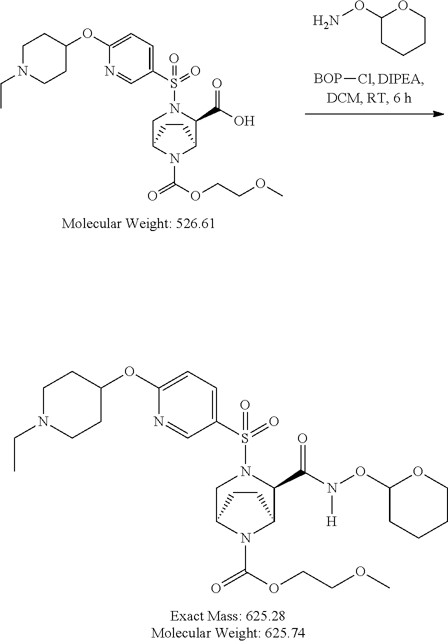

Similar procedure was followed as described for Step 3 of Example 10, to obtain 2-methoxyethyl (1S,2R,5R)-3-((6-((1-ethylpiperidin-4-yl)oxy)pyridin-3-yl)sulfonyl)-2-(((tetrahydro-2H-pyran-2-yl)oxy)carbamoyl)-3,8-diazabicyclo[3.2.1]octane-8-carboxylate (190 mg, 0.30 mmol) as a light yellow syrup. This was used as such for the next step without further purification. MS (ESI): m/z 626.3 [M+H]⁺.

### Step 6

### Synthesis of 2-methoxyethyl (1S,2R,5R)-3-((6-((1-ethylpiperidin-4-yl)oxy)pyridin-3-yl)sulfonyl)-2-(hydroxycarbamoyl)-3,8-diazabicyclo[3.2.1]octane-8-carboxylate (Example 74)

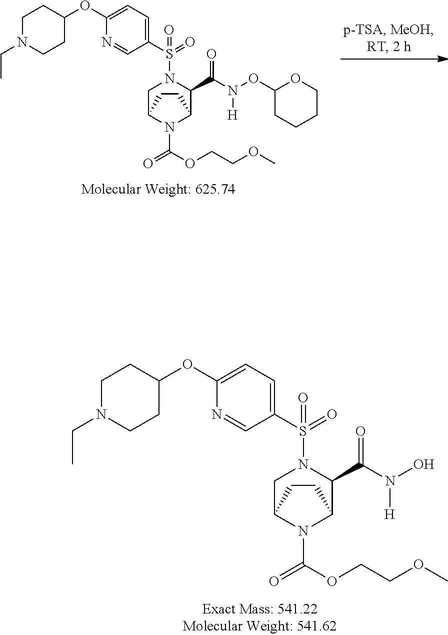 [SMILES: CCN1CCC(Oc2ccc(S(=O)(=O)N3C[C@H]4CC[C@@H]([C@@H]3C(=O)NOC3CCCCO3)N4C(=O)OCCOC)cn2)CC1 ; CCN1CCC(Oc2ccc(S(=O)(=O)N3C[C@H]4CC[C@@H]([C@@H]3C(=O)NO)N4C(=O)OCCOC)cn2)CC1]

Similar procedure was followed as described for Step 4 of Example 10 and purification was done by RP prep HPLC (Table 2, Method 4) to obtain 2-methoxyethyl (1S,2R,5R)-3-((6-((1-ethylpiperidin-4-yl)oxy)pyridin-3-yl)sulfonyl)-2-(hydroxycarbamoyl)-3, 8-diazabicyclo[3.2.1]octane-8-carboxylate (Example 74) (7 mg, 0.013 mmol, 4.3% yield for two steps) as an off-white solid. MS (ESI): m/z 542.3 [M+H]⁺; ¹H NMR (400 MHz, DMSO-d₆): δ 10.66 (brs, 1H), 8.80 (brs, 1H), 8.48-8.45 (m, 1H), 7.93-7.87 (m, 1H), 6.89 (d, J=6.8 Hz, 1H), 5.11-5.02 (m, 1H), 4.70-4.35 (m, 1H), 4.31-4.20 (m, 1H), 4.13-3.98 (m, 2H), 3.90-3.80 (m, 1H), 3.61-3.35 (m, 6H), 3.23 (s, 3H), 2.78-2.68 (m, 2H), 2.33 (q, J=3.6 Hz, 2H), 2.20-2.10 (m, 2H), 2.05-1.90 (m, 2H), 1.89-1.60 (m, 2H), 1.60-1.40 (m, 2H), 1.00 (t, J=7.2 Hz, 3H).

Similarly, below example was prepared by following the same or analogous process as described in Example 74.

TABLE 13

| Ex. | Structure | Name | Analytical data |
|---|---|---|---|
| 75 | 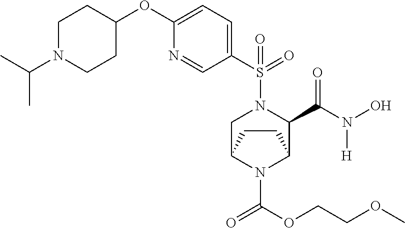 [SMILES: COCCOC(=O)N1[C@@H]2CC[C@H]1[C@H](C(=O)NO)N(S(=O)(=O)c1ccc(OC3CCN(C(C)C)CC3)nc1)C2] | 2-methoxyethyl (1S,2R,5R)-2- (hydroxycarbamoyl)- 3-((6-((1-isopropyl- piperidin-4-yl)oxy)- pyridin-3-yl)- sulfonyl)-3,8-diaza- bicyclo[3.2.1]octane- 8-carboxylate | ¹H NMR (400 MHz, DMSO- d₆): δ 10.67 (brs, 1H), 8.79 (brs, 1H), 8.46 (d, J = 2.4 Hz, 1H), 7.92 (dd, J = 2.4, 8.8 Hz, 1H), 6.87 (d, J = 10.8 Hz, 1H), 5.11-5.05 (m, 1H), 4.65- 4.35 (m, 1H), 4.30-4.20 (m, 1H), 4.12-3.99 (m, 2H), 3.95-3.80 (m, 1H), 3.61- 3.35 (m, 6H), 3.25 (s, 3H), 2.90-2.78 (m, 3H), 2.10- 1.99 (m, 2H), 1.98-1.75 (m, 4H), 1.75-1.40 (m, 2H), 1.01 (d, J = 6.4 Hz, 6H); MS (ESI): m/z 556.3 [M + H]⁺. |

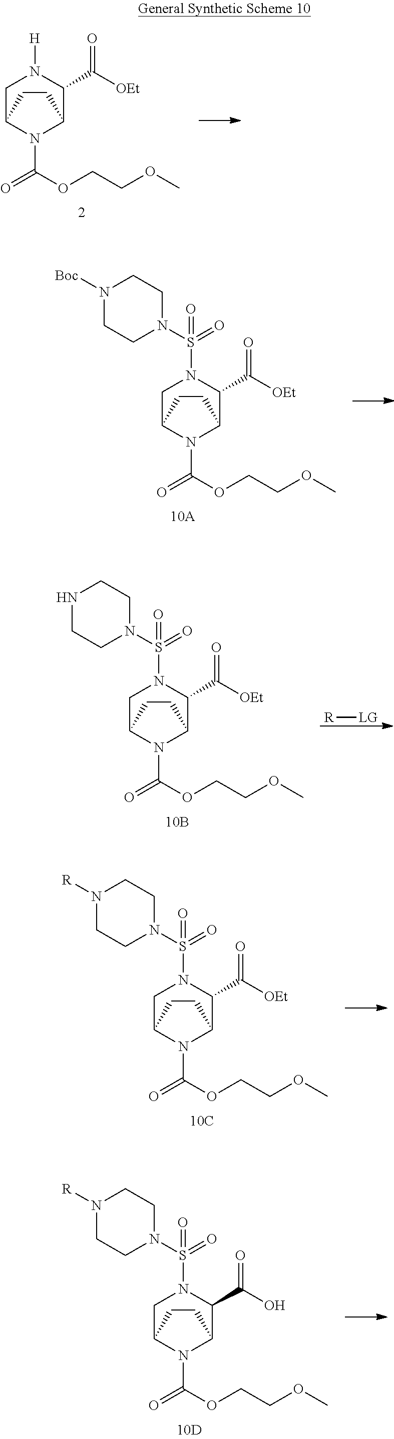

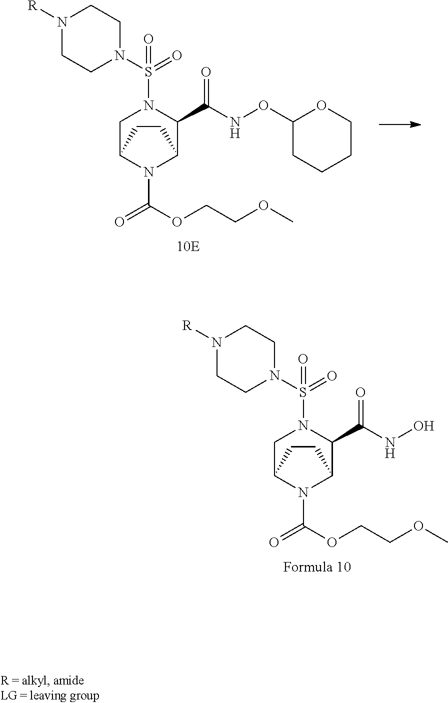

### Example 76

### Synthesis of 2-methoxyethyl (1S,2R,5R)-3-((4-(4-chlorobenzyl)piperazin-1-yl)sulfonyl)-2-(hydroxycarbamoyl)-3,8-diazabicyclo[3.2.1]octane-8-carboxylate

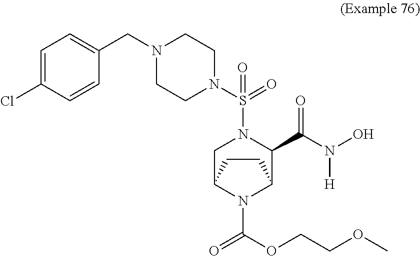 [SMILES: COCCOC(=O)N1[C@@H]2CC[C@H]1[C@H](C(=O)NO)N(S(=O)(=O)N1CCN(Cc3ccc(Cl)cc3)CC1)C2]

### Step 1

### Synthesis of 2-ethyl 8-(2-methoxyethyl) (1S,2S,5R)-3-((4-(tert-butoxycarbonyl)piperazin-1-yl)sulfonyl)-3,8-diazabicyclo[3.2.1]octane-8-dicarboxylate

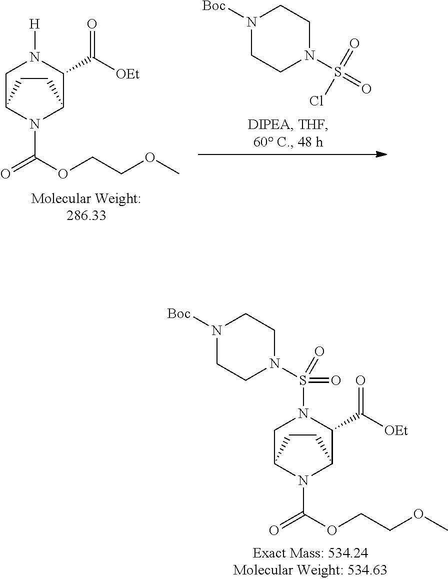 [SMILES: CCOC(=O)[C@H]1NC[C@H]2CC[C@@H]1N2C(=O)OCCOC ; CC(C)(C)OC(=O)N1CCN(S(=O)(=O)Cl)CC1 ; CCOC(=O)[C@@H]1[C@@H]2CC[C@H](CN1S(=O)(=O)N1CCN(C(=O)OC(C)(C)C)CC1)N2C(=O)OCCOC]

To a stirring solution of 2-ethyl 8-(2-methoxyethyl) (1S,2S,5R)-3,8-diazabicyclo[3.2.1]octane-2,8-dicarboxylate (2.97 g, 10.56 mmol) in THF (30 mL) under nitrogen at 0-10° C. was added DIPEA (3.62 mL, 21.12 mmol) and stirred for 20 min. tert-Butyl 4-(chlorosulfonyl)piperazine-1-carboxylate (2.0 g, 7.04 mmol) in THF (20 mL) and added and the reaction mixture was stirred at 60° C. for 48 h while being monitored by TLC. The reaction was cooled to RT, diluted with water (20 mL) and extracted with ethyl acetate (2×30 mL). The combined organic layer was dried over anhydrous Na₂SO₄, filtered and concentrated under reduced pressure to obtain crude product. The crude product was purified by silica gel column chromatography (100-200 mesh, 25-30% EtOAc in hexanes) to afford 2-ethyl 8-(2-methoxyethyl) (1S,2S,5R)-3-((4-(tert-butoxycarbonyl)piperazin-1-yl)sulfonyl)-3,8-diazabicyclo[3.2.1]octane-2,8-dicarboxylate (800 mg, 1.49 mmol, 21% yield) as a colorless gum. MS (ESI): m/z 535.3 [M+H]⁺.

### Step 2

### Synthesis of 2-ethyl 8-(2-methoxyethyl) (1S,2S,5R)-3-(piperazin-1-ylsulfonyl)-3,8-diazabicyclo[3.2.1]octane-2,8-dicarboxylate

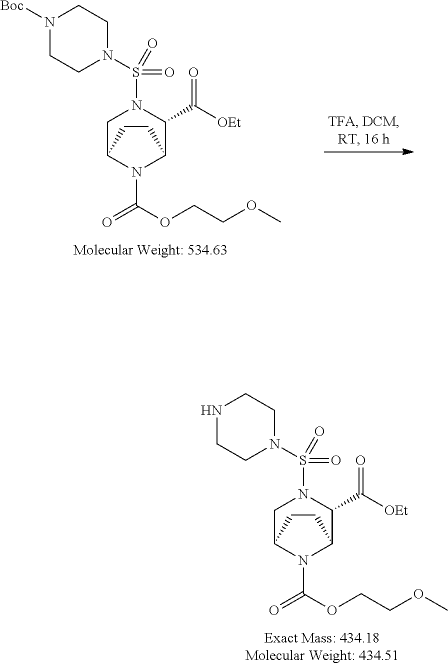 [SMILES: CCOC(=O)[C@@H]1[C@@H]2CC[C@H](CN1S(=O)(=O)N1CCN(C(=O)OC(C)(C)C)CC1)N2C(=O)OCCOC ; CCOC(=O)[C@@H]1[C@@H]2CC[C@H](CN1S(=O)(=O)N1CCNCC1)N2C(=O)OCCOC]

To a stirred solution of 2-ethyl 8-(2-methoxyethyl) (1S,2S,5R)-3-((4-(tert-butoxycarbonyl)piperazin-1-yl)sulfonyl)-3,8-diazabicyclo[3.2.1]octane-2,8-dicarboxylate (800 mg, 1.49 mmol) in DCM (10 mL) at 0-10° C. was added trifluoroacetic acid (1.13 mL, 14.9 mmol) dropwise and stirred at RT for 16 h. The completion of the reaction was monitored by TLC. The volatiles were evaporated under reduced pressure to afford 2-ethyl 8-(2-methoxyethyl) (1S,2S,5R)-3-(piperazin-1-ylsulfonyl)-3,8-diazabicyclo[3.2.1]octane-2,8-dicarboxylate (600 mg, 1.38 mmol, 92% yield) as a colorless gum. This was used as such for the next step without further purification. MS (ESI): m/z 435.2 [M+H]⁺.

### Step 3

### Synthesis of 2-ethyl 8-(2-methoxyethyl) (1S,2S,5R)-3-((4-(4-chlorobenzyl)piperazin-1-yl)sulfonyl)-3,8-diazabicyclo[3.2.1]octane-2,8-dicarboxylate

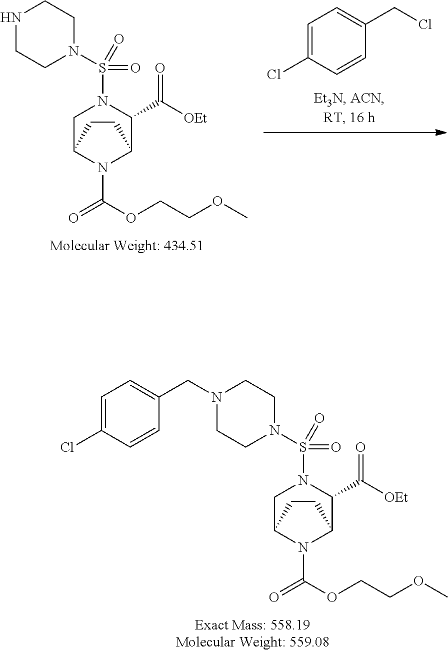 [SMILES: CCOC(=O)[C@@H]1[C@@H]2CC[C@H](CN1S(=O)(=O)N1CCNCC1)N2C(=O)OCCOC ; ClCc1ccc(Cl)cc1 ; CCOC(=O)[C@@H]1[C@@H]2CC[C@H](CN1S(=O)(=O)N1CCN(Cc3ccc(Cl)cc3)CC1)N2C(=O)OCCOC]

To a stirred solution of 2-ethyl 8-(2-methoxyethyl) (1S,2S,5R)-3-(piperazin-1-ylsulfonyl)-3,8-diazabicyclo[3.2.1]octane-2,8-dicarboxylate (250 mg, 0.57 mmol) in ACN (3 mL) at 0-5° C. was added Et₃N (0.4 mL, 2.87 mmol) and 1-chloro-4-(chloromethyl)benzene (0.13 mL, 0.86 mmol). The reaction was stirred at RT for 16 h while being monitored by TLC. The reaction was diluted with ethyl acetate (20 mL), washed with aqueous saturated NaHCO₃ solution (2×10 mL). The organic layer was separated, dried over anhydrous Na₂SO₄, filtered and concentrated under reduced pressure to obtain crude product. The crude product was purified by silica gel column chromatography (100-200 mesh, 2-3% MeOH in DCM) to afford 2-ethyl 8-(2-methoxyethyl) (1S,2S,5R)-3-((4-(4-chlorobenzyl)piperazin-1-yl)sulfonyl)-3,8-diazabicyclo[3.2.1]octane-2,8-dicarboxylate (200 mg, 0.35 mmol, 61% yield) as a colorless gum. MS (ESI): m/z 559.2 [M+H]⁺.

### Step 4

### Synthesis of (1S,2R,5R)-3-((4-(4-chlorobenzyl)piperazin-1-yl)sulfonyl)-8-((2-methoxyethoxy)carbonyl)-3,8-diazabicyclo[3.2.1]octane-2-carboxylic acid

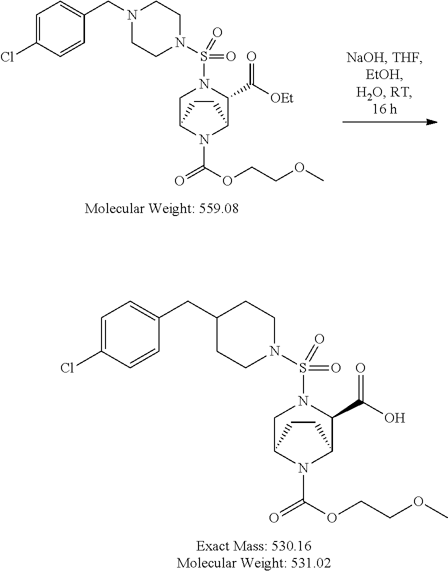 [SMILES: CCOC(=O)[C@@H]1[C@@H]2CC[C@H](CN1S(=O)(=O)N1CCN(Cc3ccc(Cl)cc3)CC1)N2C(=O)OCCOC ; COCCOC(=O)N1[C@@H]2CC[C@H]1[C@H](C(=O)O)N(S(=O)(=O)N1CCC(Cc3ccc(Cl)cc3)CC1)C2]

Similar procedure was followed as described for Step 4 of Example 1, to obtain (1S,2R,5R)-3-((4-(4-chlorobenzyl)piperazin-1-yl)sulfonyl)-8-((2-methoxyethoxy)carbonyl)-3, 8-diazabicyclo[3.2.1]octane-2-carboxylic acid (150 mg, 0.28 mmol, 83% yield) as a brown gum. This was used as such for the next step without further purification. MS (ESI): m/z 531.2 [M+H]⁺.

### Step 5

### Synthesis of 2-methoxyethyl (1S,2R,5R)-3-((4-(4-chlorobenzyl)piperazin-1-yl)sulfonyl)-2-(((tetrahydro-2H-pyran-2-yl)oxy)carbamoyl)-3,8-diazabicyclo[3.2.1]octane-8-carboxylate

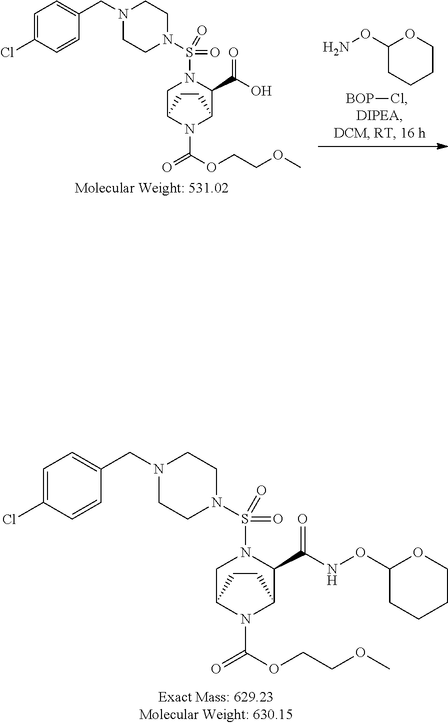

Similar procedure was followed as described for Step 3 of Example 10, to obtain 2-methoxyethyl (1S,2R,5R)-3-((4-(4-chlorobenzyl)piperazin-1-yl)sulfonyl)-2-(((tetrahydro-2H-pyran-2-yl)oxy)carbamoyl)-3,8-diazabicyclo[3.2.1]octane-8-carboxylate (160 mg, 0.25 mmol, 96% yield) as a colorless gum. This was used as such for the next step without further purification. MS (ESI): m/z 630.2 [M+H]⁺.

### Step 6

### Synthesis of 2-methoxyethyl (1S,2R,5R)-3-((4-(4-chlorobenzyl)piperazin-1-yl)sulfonyl)-2-(hydroxycarbamoyl)-3,8-diazabicyclo[3.2.1]octane-8-carboxylate (Example 76)

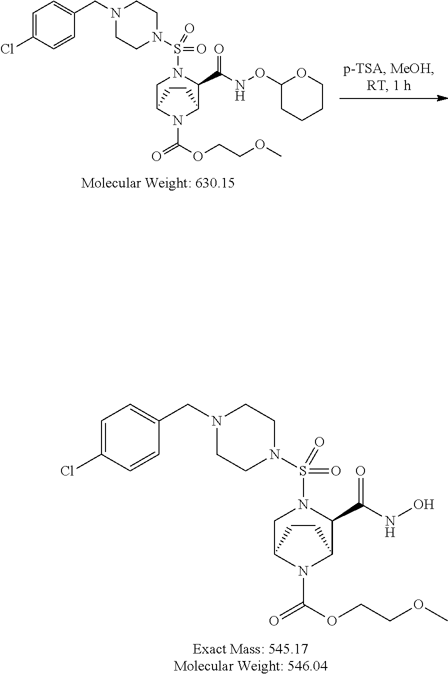 [SMILES: COCCOC(=O)N1[C@@H]2CC[C@H]1[C@H](C(=O)NOC1CCCCO1)N(S(=O)(=O)N1CCN(Cc3ccc(Cl)cc3)CC1)C2 ; COCCOC(=O)N1[C@@H]2CC[C@H]1[C@H](C(=O)NO)N(S(=O)(=O)N1CCN(Cc3ccc(Cl)cc3)CC1)C2]

Similar procedure was followed as described for Step 4 of Example 10 and purification was done by RP prep HPLC (Table 2, Method 6) to obtain 2-methoxyethyl (1S,2R,5R)-3-((4-(4-chlorobenzyl)piperazin-1-yl)sulfonyl)-2-(hydroxycarbamoyl)-3, 8-diazabicyclo[3.2.1]octane-8-carboxylate (Example 76) (11 mg, 0.02 mmol, 8% yield) as an off-white solid. MS (ESI): m/z 546.1 [M+H]⁺; ¹H NMR (400 MHz, DMSO-d₆): δ 7.40-7.36 (m, 2H), 7.34-7.30 (m, 2H), 4.48-4.32 (m, 1H), 4.31-4.19 (m, 1H), 4.18-4.05 (m, 2H), 3.98-3.75 (m, 1H), 3.75-3.45 (m, 4H), 3.26 (s, 3H), 3.22-3.10 (m, 2H), 3.10-3.01 (m, 4H), 2.40-2.30 (m, 4H), 2.01-1.72 (m, 2H), 1.71-1.60 (m, 2H); —NH and —OH peaks were not observed.

Similarly, below example was prepared by following the same or analogous process as described in Example 76.

TABLE 14

| Ex. | Structure | Name | Analytical data |
|---|---|---|---|
| 77 | 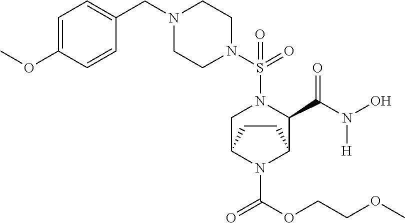 | 2-methoxyethyl (1S,2R,5R)-2- (hydroxycarbamoyl)- 3-((4-(4- methoxybenzyl)- piperazin-1-yl)- sulfonyl)-3,8-diaza- bicyclo[3.2.1]octane- 8-carboxylate | ¹H NMR (400 MHz, DMSO- d₆): δ 10.60 (brs, 1H), 8.83 (brs, 1H), 7.21-7.18 (m, 2H), 6.89-6.86 (m, 2H), 4.70- 4.30 (m, 1H), 4.30-4.02 (m, 3H), 3.98-3.75 (m, 3H), 3.73 (s, 3H), 3.70-3.35 (m, 4H), 3.26 (s, 3H), 3.10-3.00 (m, 4H), 2.40-2.30 (m, 4H), 2.01- 1.75 (m, 2H), 1.73-1.45 (m, 2H); MS (ESI): m/z 542.2 [M + H]⁺. |

### Example 78

### Synthesis of 2-methoxyethyl (1S,2R,5R)-3-((4-(4-chlorobenzoyl)piperazin-1-yl)sulfonyl)-2-(hydroxycarbamoyl)-3,8-diazabicyclo[3.2.1]octane-8-carboxylate

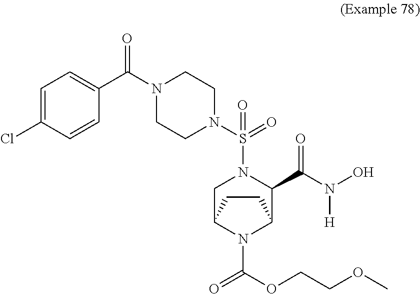 [SMILES: COCCOC(=O)N1[C@@H]2CC[C@H]1[C@H](C(=O)NO)N(S(=O)(=O)N1CCN(C(=O)c3ccc(Cl)cc3)CC1)C2]

### Step 1

### Synthesis of 2-ethyl 8-(2-methoxyethyl) (1S,2S,5R)-3-((4-(4-chlorobenzoyl)piperazin-1-yl)sulfonyl)-3,8-diazabicyclo[3.2.1]octane-2,8-dicarboxylate

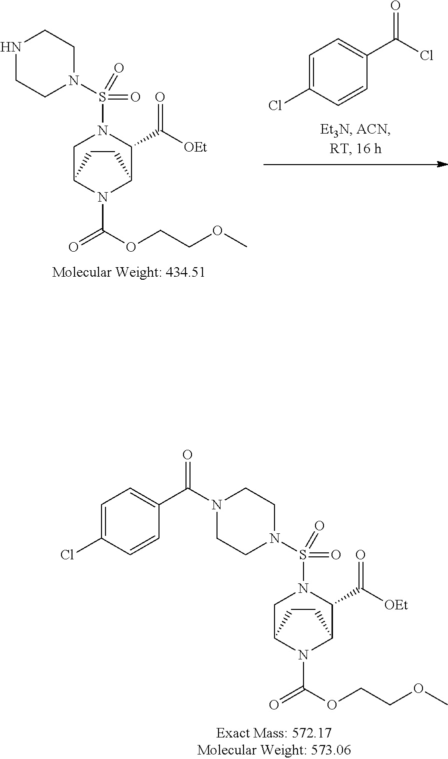 [SMILES: CCOC(=O)[C@@H]1[C@@H]2CC[C@H](CN1S(=O)(=O)N1CCNCC1)N2C(=O)OCCOC ; O=C(Cl)c1ccc(Cl)cc1 ; CCOC(=O)[C@@H]1[C@@H]2CC[C@H](CN1S(=O)(=O)N1CCN(C(=O)c3ccc(Cl)cc3)CC1)N2C(=O)OCCOC]

To a stirred solution of 2-ethyl 8-(2-methoxyethyl) (1S,2S,5R)-3-(piperazin-1-ylsulfonyl)-3,8-diazabicyclo[3.2.1]octane-2,8-dicarboxylate (250 mg, 0.57 mmol) in ACN (3 mL) at 0-5° C. was added Et₃N (0.4 mL, 2.87 mmol) followed by 4-chlorobenzoyl chloride (0.11 mL, 0.86 mmol). The reaction was stirred at RT for 16 h while being monitored by TLC. The reaction was diluted with ethyl acetate (20 mL), washed with water (2×10 mL). The organic layer was separated, dried over anhydrous Na₂SO₄, filtered and concentrated under reduced pressure to obtain crude product. The crude product was purified by silica gel column chromatography (100-200 mesh, 2-3% MeOH in DCM) to afford 2-ethyl 8-(2-methoxyethyl) (1S,2S,5R)-3-((4-(4-chlorobenzoyl)piperazin-1-yl)sulfonyl)-3,8-diazabicyclo[3.2.1]octane-2,8-dicarboxylate (180 mg, 0.31 mmol, 54% yield) as a colorless gum. MS (ESI): m/z 573.1 [M+H]⁺.

### Step 2

### Synthesis of (1S,2R,5R)-3-((4-(4-chlorobenzoyl)piperazin-1-yl)sulfonyl)-8-((2-methoxyethoxy)carbonyl)-3,8-diazabicyclo[3.2.1]octane-2-carboxylic acid

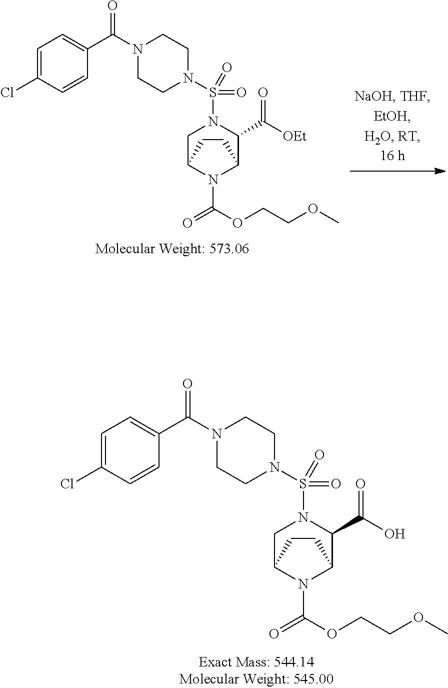 [SMILES: CCOC(=O)[C@@H]1[C@@H]2CC[C@H](CN1S(=O)(=O)N1CCN(C(=O)c3ccc(Cl)cc3)CC1)N2C(=O)OCCOC ; COCCOC(=O)N1[C@@H]2CC[C@H]1[C@H](C(=O)O)N(S(=O)(=O)N1CCN(C(=O)c3ccc(Cl)cc3)CC1)C2]

Similar procedure was followed as described for Step 4 of Example 1, to obtain (1S,2R,5R)-3-((4-(4-chlorobenzoyl)piperazin-1-yl)sulfonyl)-8-((2-methoxyethoxy)carbonyl)-3,8-diazabicyclo[3.2.1]octane-2-carboxylic acid (110 mg, 0.20 mmol, 68% yield) as a brown gum. This was used as such for the next step without further purification. MS (ESI): m/z 545.1 [M+H]⁺.

### Step 3

### Synthesis of 2-methoxyethyl (1S,2R,5R)-3-((4-(4-chlorobenzoyl)piperazin-1-yl)sulfonyl)-2-(((tetrahydro-2H-pyran-2-yl)oxy)carbamoyl)-3,8-diazabicyclo[3.2.1]octane-8-carboxylate

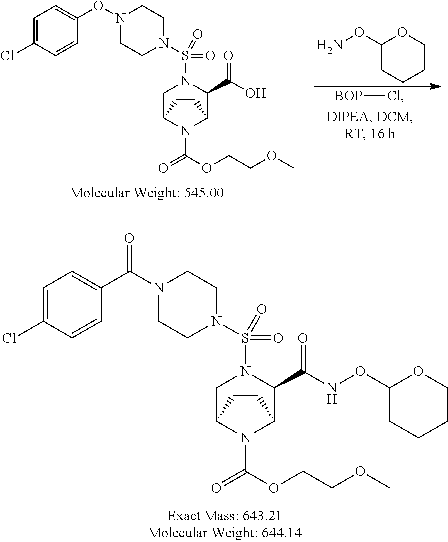

Similar procedure was followed as described for Step 3 of Example 10, to obtain 2-methoxyethyl (1S,2R,5R)-3-((4-(4-chlorobenzoyl)piperazin-1-yl)sulfonyl)-2-(((tetrahydro-2H-pyran-2-yl)oxy)carbamoyl)-3,8-diazabicyclo[3.2.1]octane-8-carboxylate (120 mg, 0.18 mmol, quantitative) as a colorless gum. This was used as such for the next step without further purification. MS (ESI): m/z 642.2 [M−H]⁻.

### Step 4

### Synthesis of 2-methoxyethyl (1S,2R,5R)-3-((4-(4-chlorobenzoyl)piperazin-1-yl)sulfonyl)-2-(hydroxycarbamoyl)-3,8-diazabicyclo[3.2.1]octane-8-carboxylate (Example 78)

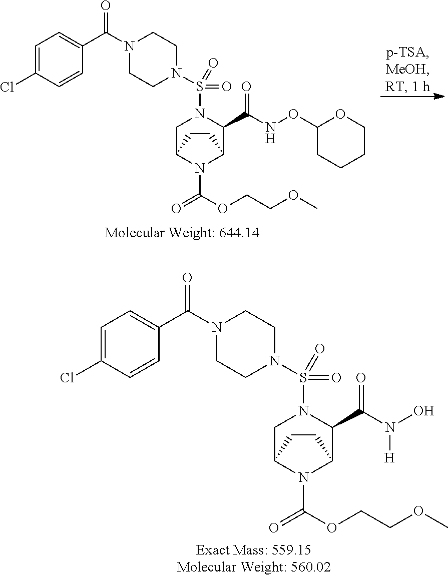 [SMILES: COCCOC(=O)N1[C@@H]2CC[C@H]1[C@H](C(=O)NOC1CCCCO1)N(S(=O)(=O)N1CCN(C(=O)c3ccc(Cl)cc3)CC1)C2 ; COCCOC(=O)N1[C@@H]2CC[C@H]1[C@H](C(=O)NO)N(S(=O)(=O)N1CCN(C(=O)c3ccc(Cl)cc3)CC1)C2]

Similar procedure was followed as described for Step 4 of Example 10 and purification was done by RP prep HPLC (Table 2, Method 1) to obtain 2-methoxyethyl (1S,2R,5R)-3-((4-(4-chlorobenzoyl)piperazin-1-yl)sulfonyl)-2-(hydroxycarbamoyl)-3, 8-diazabicyclo[3.2.1]octane-8-carboxylate (Example 78) (3.7 mg, 0.006 mmol, 3% yield for two steps) as an off-white solid. MS (ESI): m/z 560.1 [M+H]⁺; ¹H NMR (400 MHz, DMSO-d₆): δ 10.66 (brs, 1H), 8.84 (brs, 1H), 7.52 (d, J=8.4 Hz, 2H), 7.44 (d, J=8.4 Hz, 2H), 4.70-4.35 (m, 1H), 4.31-4.20 (m, 1H), 4.11-4.01 (m, 2H), 3.99-3.76 (m, 2H), 3.70-3.35 (m, 7H), 3.25 (s, 3H), 3.19-3.00 (m, 4H), 2.01-1.75 (m, 2H), 1.74-1.60 (m, 2H).

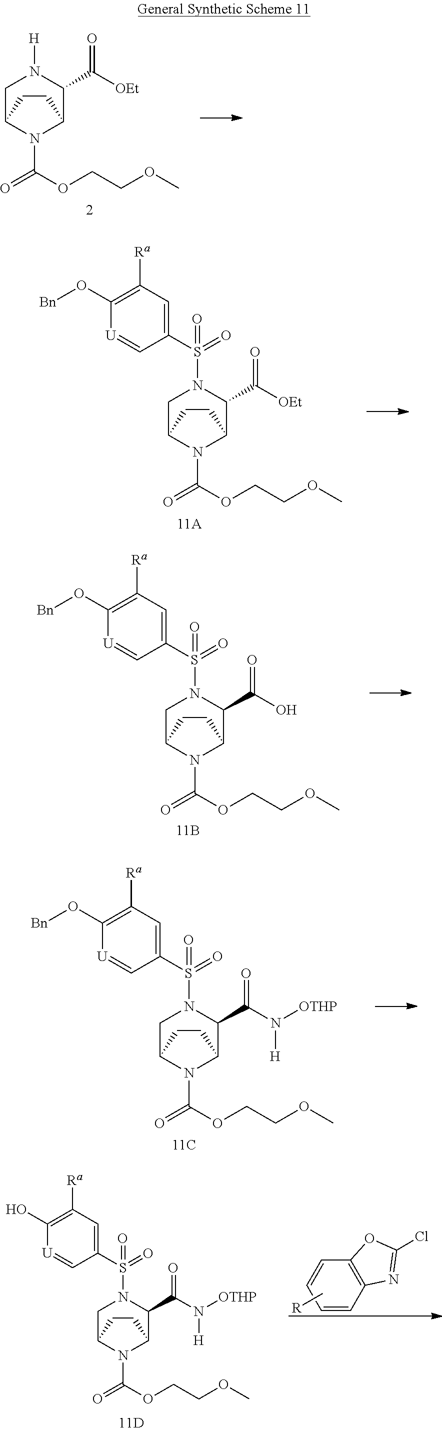

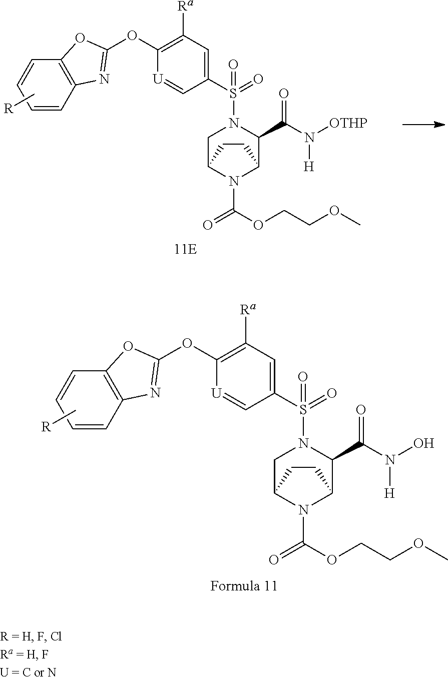

### Example 79

### Synthesis of 2-methoxyethyl (1S,2R,5R)-3-((4-(benzo[d]oxazol-2-yloxy)-3-fluorophenyl)sulfonyl)-2-(hydroxycarbamoyl)-3,8-diazabicyclo[3.2.1]octane-8-carboxylate

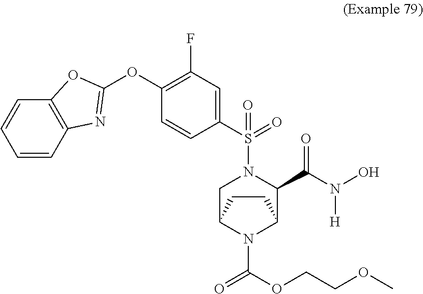 [SMILES: COCCOC(=O)N1[C@@H]2CC[C@H]1[C@H](C(=O)NO)N(S(=O)(=O)c1ccc(Oc3nc4ccccc4o3)c(F)c1)C2]

### Step 1

### Synthesis of 2-ethyl 8-(2-methoxyethyl) (2S,5R)-3-((4-(benzyloxy)-3-fluorophenyl)sulfonyl)-3,8-diazabicyclo[3.2.1]octane-2,8-dicarboxylate

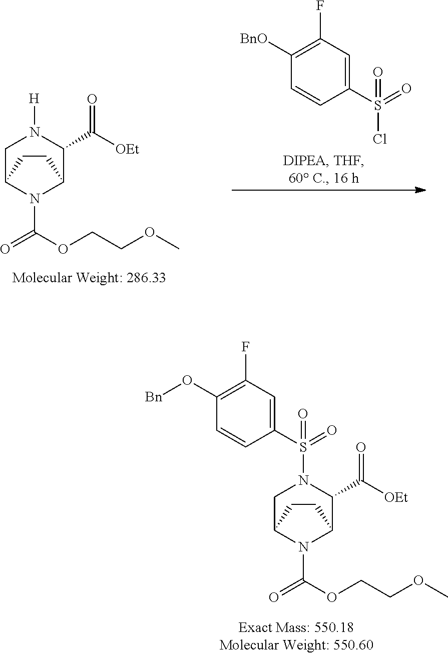 [SMILES: CCOC(=O)[C@H]1NC[C@H]2CC[C@@H]1N2C(=O)OCCOC ; O=S(=O)(Cl)c1ccc(OCc2ccccc2)c(F)c1 ; CCOC(=O)[C@@H]1[C@@H]2CC[C@H](CN1S(=O)(=O)c1ccc(OCc3ccccc3)c(F)c1)N2C(=O)OCCOC]

To a stirring solution of 2-ethyl 8-(2-methoxyethyl) (1S,2S,5R)-3,8-diazabicyclo[3.2.1]octane-2,8-dicarboxylate (1 g, 3.49 mmol) in THF (5 mL) under nitrogen at 0-10° C. was added DIPEA (1.8 mL, 10.5 mmol) and stirred for 20 min. 4-(Benzyloxy)-3-fluorobenzenesulfonyl chloride (1.57 g, 5.23 mmol) in THF (10 mL) was added and the reaction mixture was stirred at 60° C. for 16 h while being monitored by TLC. The reaction was cooled to RT, diluted with water (10 mL) and extracted with ethyl acetate (2×25 mL). The combined organic layer was dried over anhydrous Na₂SO₄, filtered and concentrated under reduced pressure to obtain crude product. The crude product was purified by silica gel column chromatography (100-200 mesh, 25-30% EtOAc in hexanes) to afford 2-ethyl 8-(2-methoxyethyl) (2S,5R)-3-((4-(benzyloxy)-3-fluorophenyl)sulfonyl)-3,8-diazabicyclo[3.2.1]-octane-2,8-dicarboxylate (900 mg, 1.63 mmol, 47% yield) as a colorless gum. MS (ESI): m/z 551.2 [M+H]⁺.

### Step 2

### Synthesis of (2R,5R)-3-((4-(benzyloxy)-3-fluorophenyl)sulfonyl)-8-((2-methoxyethoxy)carbonyl)-3,8-diazabicyclo[3.2.1]octane-2-carboxylic acid

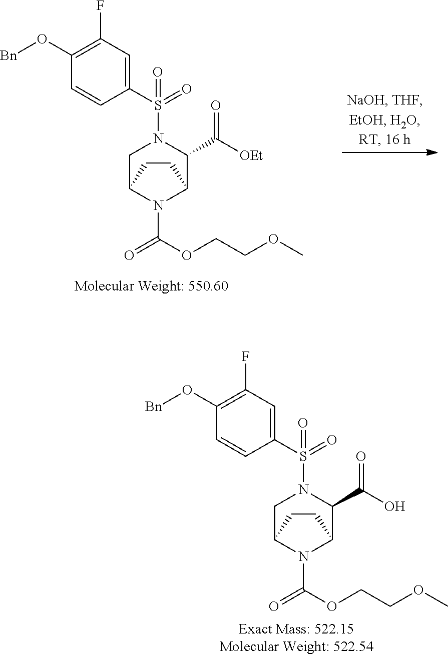 [SMILES: CCOC(=O)[C@@H]1[C@@H]2CC[C@H](CN1S(=O)(=O)c1ccc(OCc3ccccc3)c(F)c1)N2C(=O)OCCOC ; COCCOC(=O)N1[C@@H]2CC[C@H]1[C@H](C(=O)O)N(S(=O)(=O)c1ccc(OCc3ccccc3)c(F)c1)C2]

Similar procedure was followed as described for Step 4 of Example 1, to obtain (2R,5R)-3-((4-(benzyloxy)-3-fluorophenyl)sulfonyl)-8-((2-methoxyethoxy)carbonyl)-3,8-diazabicyclo[3.2.1]octane-2-carboxylic acid (700 mg, 1.33 mmol, 81% yield) as a colorless sticky gum. This was used as such for the next step without further purification. MS (ESI): m/z 523.1 [M+H]⁺.

### Step 3

### Synthesis of 2-methoxyethyl (2R,5R)-3-((4-(benzyloxy)-3-fluorophenyl)sulfonyl)-2-(((tetrahydro-2H-pyran-2-yl)oxy)carbamoyl)-3,8-diazabicyclo[3.2.1]octane-8-carboxylate

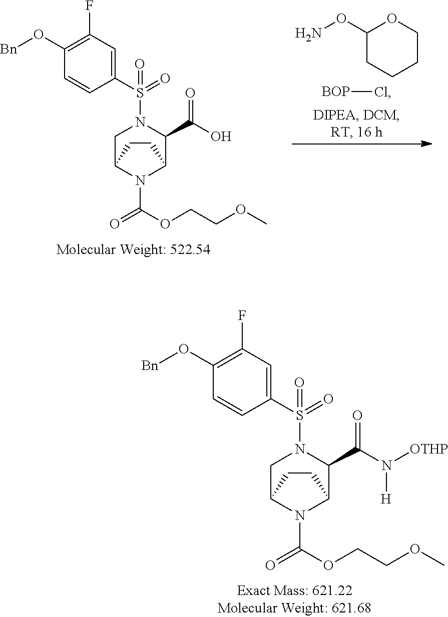

Similar procedure was followed as described for Step 3 of Example 10, to obtain 2-methoxyethyl (2R,5R)-3-((4-(benzyloxy)-3-fluorophenyl)sulfonyl)-2-(((tetrahydro-2H-pyran-2-yl)oxy)carbamoyl)-3,8-diazabicyclo[3.2.1]octane-8-carboxylate (600 mg, 0.96 mmol, 72% yield) as a colorless oil. This was used as such for the next step without further purification. MS (ESI): m/z 620.1 [M−H]⁻.

### Step 4

### Synthesis of 2-methoxyethyl (2R,5R)-3-((3-fluoro-4-hydroxyphenyl)sulfonyl)-2-(((tetrahydro-2H-pyran-2-yl)oxy)carbamoyl)-3,8-diazabicyclo[3.2.1]octane-8-carboxylate

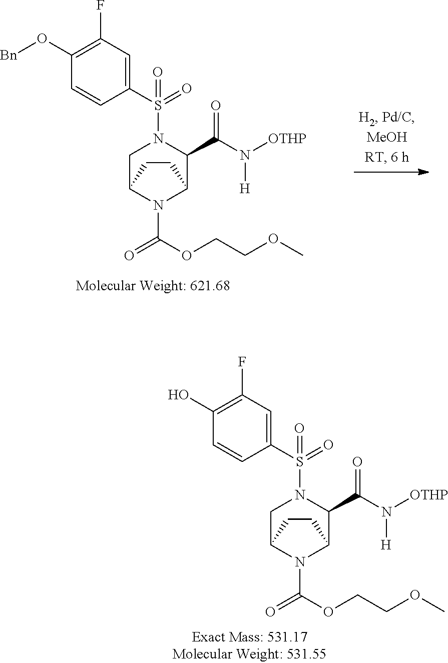

To a stirred solution of 2-methoxyethyl (2R,5R)-3-((4-(benzyloxy)-3-fluoro-phenyl)sulfonyl)-2-(((tetrahydro-2H-pyran-2-yl)oxy)carbamoyl)-3,8-diazabicyclo[3.2.1]octane-8-carboxylate (600 mg, 0.96 mmol) in MeOH (10 mL) was added Pd/C (180 mg, 30% w/w) under N2 atmosphere. The reaction was stirred under H2 balloon pressure for 6 h while being monitored by TLC. Reaction mixture was filtered through a celite bed and washed with MeOH (20 mL). The filtrate was evaporated under reduced pressure to afford 2-methoxyethyl (2R,5R)-3-((3-fluoro-4-hydroxyphenyl)sulfonyl)-2-(((tetrahydro-2H-pyran-2-yl)oxy)carbamoyl)-3,8-diazabicyclo[3.2.1]octane-8-carboxylate (500 mg, 0.94 mmol, quantitative) as colorless liquid; MS (ESI): m/z 530.1 [M−H]⁻.

### Step 5

### Synthesis of 2-methoxyethyl (2R,5R)-3-((4-(benzo[d]oxazol-2-yloxy)-3-fluorophenyl)-sulfonyl)-2-(((tetrahydro-2H-pyran-2-yl)oxy)carbamoyl)-3,8-diazabicyclo[3.2.1]octane-8-carboxylate

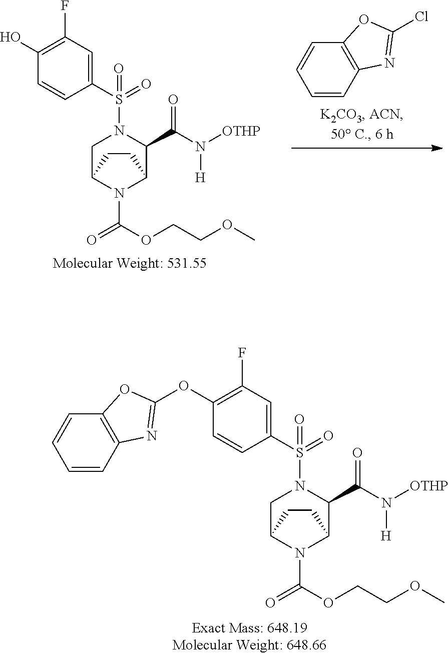

To a stirring solution of 2-methoxyethyl (2R,5R)-3-((3-fluoro-4-hydroxyphenyl)sulfonyl)-2-(((tetrahydro-2H-pyran-2-yl)oxy)carbamoyl)-3,8-diazabicyclo[3.2.1]octane-8-carboxylate (200 mg, 0.37 mmol) in ACN (4 mL) was added K₂CO₃ (155 mg, 1.13 mmol) under nitrogen at 0-10° C. and stirred for 20 min. 2-Chlorobenzo[d]oxazole (63 mg, 0.41 mmol) in ACN (2 mL) was added to the reaction and stirred at 50° C. for 6 h while being monitored by TLC. The reaction was cooled to room temperature, diluted with water (5 mL) and extracted with ethyl acetate (2×20 mL). The combined organic layer was dried over anhydrous Na₂SO₄, filtered and concentrated under reduced pressure. The crude product was purified by silica gel column chromatography (100-200 mesh, 50-80% EtOAc in hexanes) to afford 2-methoxyethyl (2R,5R)-3-((4-(benzo[d]oxazol-2-yloxy)-3-fluorophenyl)sulfonyl)-2-(((tetrahydro-2H-pyran-2-yl)oxy)carbamoyl)-3,8-diazabicyclo[3.2.1]octane-8-carboxylate (120 mg, 0.18 mmol, 50% yield) as a colorless gum; MS (ESI): m/z 649.4 [M+H]⁺.

### Step 6

### Synthesis of 2-methoxyethyl (1S,2R,5R)-3-((4-(benzo[d]oxazol-2-yloxy)-3-fluorophenyl)-sulfonyl)-2-(hydroxycarbamoyl)-3,8-diazabicyclo[3.2.1]octane-8-carboxylate (Example 79)

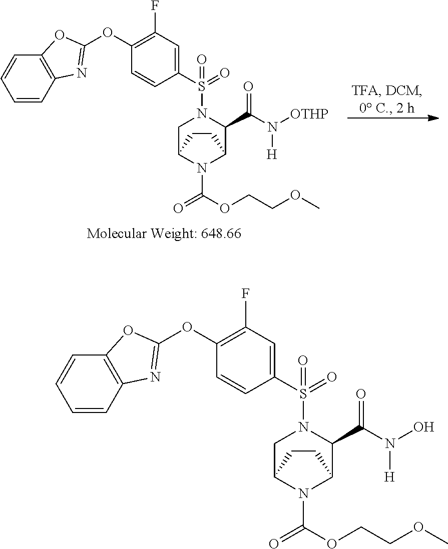

To a stirred solution of 2-methoxyethyl (2R,5R)-3-((4-(benzo[d]oxazol-2-yloxy)-3-fluorophenyl)sulfonyl)-2-(((tetrahydro-2H-pyran-2-yl)oxy)carbamoyl)-3,8-diazabicyclo[3.2.1]octane-8-carboxylate (120 mg, 0.18 mmol) in DCM (4 mL) was added trifluoroacetic acid (0.14 mL, 1.84 mmol) dropwise at 0-10° C. and stirred for 2 h while being monitored by TLC. The reaction was quenched with aqueous saturated NaHCO₃ solution (10 mL) and was extracted with DCM (2×20 mL). The organic layer was separated, dried over anhydrous Na₂SO₄, filtered, and concentrated under reduced pressure to afford crude product. The crude compound was purified by Biotage RP purification using 0.1% formic acid in water and ACN, and pure fractions were lyophilized to afford 2-methoxyethyl (1S,2R,5R)-3-((4-(benzo[d]oxazol-2-yloxy)-3-fluorophenyl)sulfonyl)-2-(hydroxycarbamoyl)-3,8-diazabicyclo[3.2.1]octane-8-carboxylate (Example 79) (9 mg, 0.016 mmol, 9% yield) as an off-white solid. MS (ESI): m/z 565.1 [M+H]⁺; 1H NMR (400 MHz, DMSO-d6): δ 10.68 (brs, 1H), 8.83 (brs, 1H), 8.05-8.01 (m, 1H), 7.93-7.86 (m, 1H), 7.75-7.68 (m, 2H), 7.59-7.56 (m, 1H), 7.38-7.34 (m, 2H), 4.70-4.40 (m, 1H), 4.32-4.20 (m, 1H), 4.18-3.80 (m, 3H), 3.70-3.40 (m, 4H), 3.24 (s, 3H), 2.01-1.75 (m, 2H), 1.70-1.41 (m, 2H).

Similarly, below examples were prepared by following the same or analogous process as described in Example 79.

TABLE 15

| Ex. | Structure | Name | Analytical data |
|---|---|---|---|
| 80 | 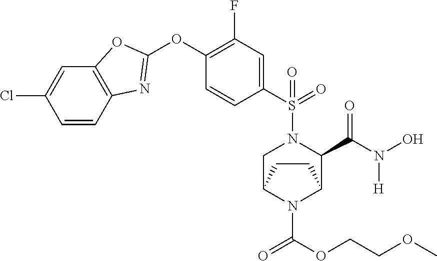 [SMILES: COCCOC(=O)N1[C@@H]2CC[C@H]1[C@H](C(=O)NO)N(S(=O)(=O)c1ccc(Oc3nc4ccc(Cl)cc4o3)c(F)c1)C2] | 2-methoxyethyl (1S,2R,5R)-3-((4- ((6-chlorobenzo[d]- oxazol-2-yl)oxy)-3- fluorophenyl)- sulfonyl)-2- (hydroxycarbamoyl)- 3,8-diazabicyclo- [3.2.1]octane-8- carboxylate | ¹H NMR (400 MHz, DMSO- d₆): δ 10.68 (brs, 1H), 8.84 (brs, 1H), 8.08 (t, J = 8.0 Hz, 1H), 7.98 (d, J = 3.2 Hz, 1H), 7.91-7.86 (m, 1H), 7.74 (d, J = 8.4 Hz, 1H), 7.59 (d, J = 8.8 Hz, 1H), 7.43 (dd, J = 2.8, 5.2 Hz, 1H), 4.66-4.40 (m, 1H), 4.32-4.22 (m, 1H), 4.16- 3.80 (m, 2H), 3.70-3.40 (m, 5H), 3.24 (s, 3H), 2.01-1.75 (m, 2H), 1.70-1.42 (m, 2H); MS (ESI): m/z 599.2 [M + H]⁺. |
| 81 | 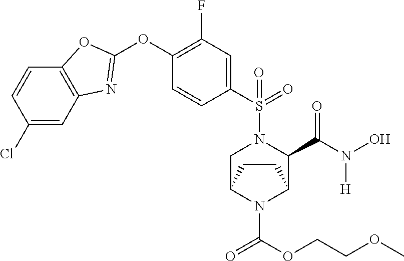 [SMILES: COCCOC(=O)N1[C@@H]2CC[C@H]1[C@H](C(=O)NO)N(S(=O)(=O)c1ccc(Oc3nc4cc(Cl)ccc4o3)c(F)c1)C2] | 2-methoxyethyl (1S,2R,5R)-3-((4- ((5-chlorobenzo[d]- oxazol-2-yl)oxy)-3- fluorophenyl) sulfonyl)-2- (hydroxy- carbamoyl)-3,8- diazabicyclo[3.2.1]- octane-8-carboxylate | ¹H NMR (400 MHz, DMSO- d₆): δ 10.69 (brs, 1H), 8.29 (brs, 1H), 8.02 (t, J = 8.4 Hz, 1H), 7.90 (d, J = 8.8 Hz, 1H), 7.76-7.71 (m, 3H), 7.40 (dd, J = 2.4, 8.6 Hz, 1H), 4.67- 4.39 (m, 1H), 4.30-4.20 (m, 1H), 4.17-3.80 (m, 2H), 3.70- 3.40 (m, 5H), 3.24 (s, 3H), 2.02-1.74 (m, 2H), 1.65- 1.42 (m, 2H); MS (ESI): m/z 599.1 [M + H]⁺. |
| 82 | 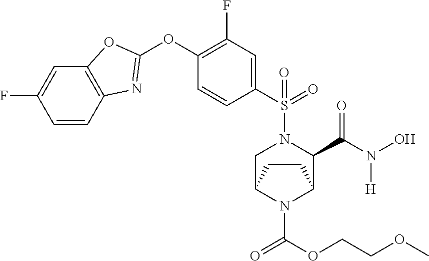 [SMILES: COCCOC(=O)N1[C@@H]2CC[C@H]1[C@H](C(=O)NO)N(S(=O)(=O)c1ccc(Oc3nc4ccc(F)cc4o3)c(F)c1)C2] | 2-methoxyethyl (1S,2R,5R)-3-((3- fluoro-4-((6-fluoro- benzo[d]oxazol-2- yl)oxy)phenyl)- sulfonyl)-2- (hydroxycarbamoyl)- 3,8-diazabicyclo- [3.2.1]octane-8- carboxylate | ¹H NMR (400 MHz, DMSO- d₆): δ 10.67 (brs, 1H), 8.82 (brs, 1H), 8.02 (t, J = 8.4 Hz, 1H), 7.89 (d, J = 8.8 Hz, 1H), 7.77-7.72 (m, 2H), 7.62- 7.58 (m, 1H), 7.26-7.21 (m, 1H), 4.66-4.40 (m, 1H), 4.31- 4.20 (m, 1H), 4.18-3.80 (m, 3H), 3.70-3.40 (m, 4H), 3.18 (s, 3H), 2.01-1.72 (m, 2H), 1.66-1.41 (m, 2H); MS (ESI): m/z 583.1 [M + H]⁺. |
| 83 | 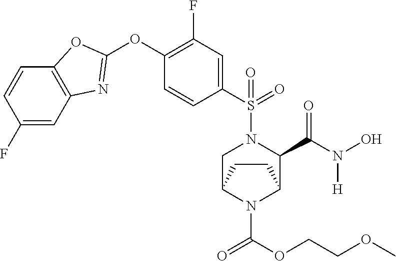 [SMILES: COCCOC(=O)N1[C@@H]2CC[C@H]1[C@H](C(=O)NO)N(S(=O)(=O)c1ccc(Oc3nc4cc(F)ccc4o3)c(F)c1)C2] | 2-methoxyethyl (1S,2R,5R)-3-((3- fluoro-4-((5-fluoro- benzo[d]oxazol-2- yl)oxy)phenyl) sulfonyl)-2- (hydroxy- carbamoyl)-3,8- diazabicyclo[3.2.1]- octane-8-carboxylate | ¹H NMR (400 MHz, DMSO- d₆): δ 10.68 (brs, 1H), 8.82 (brs, 1H), 8.02 (t, J = 8.8 Hz, 1H), 7.80 (d, J = 9.2 Hz, 1H), 7.76-7.72 (m, 2H), 7.50 (dd, J = 2.8, 8.8 Hz, 1H), 7.23- 7.12 (m, 1H), 4.67-4.40 (m, 1H), 4.31-4.21 (m, 1H), 4.19- 3.80 (m, 3H), 3.69-3.40 (m, 4H), 3.23 (s, 3H), 2.01- 1.75 (m, 2H), 1.65-1.41 (m, 2H); MS (ESI): m/z 583.1 [M + H]⁺. |

### Example 84

### Synthesis of 2-methoxyethyl (1S,2R,5R)-3-((6-(benzo[d]oxazol-2-yloxy)pyridin-3-yl)sulfonyl)-2-(hydroxycarbamoyl)-3,8-diazabicyclo[3.2.1]octane-8-carboxylate

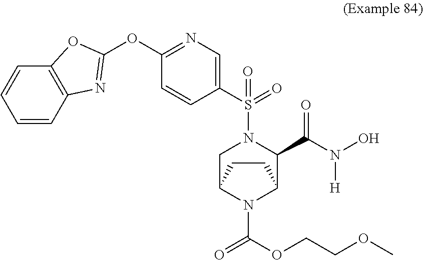 [SMILES: COCCOC(=O)N1[C@@H]2CC[C@H]1[C@H](C(=O)NO)N(S(=O)(=O)c1ccc(Oc3nc4ccccc4o3)nc1)C2]

### Step 1

### Synthesis of 2-ethyl 8-(2-methoxyethyl) (2S,5R)-3-((6-(benzyloxy)pyridin-3-yl)sulfonyl)-3,8-diazabicyclo[3.2.1]octane-2,8-dicarboxylate

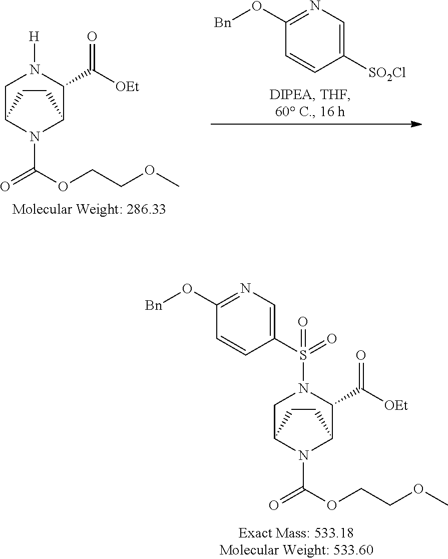 [SMILES: CCOC(=O)[C@H]1NC[C@H]2CC[C@@H]1N2C(=O)OCCOC ; O=S(=O)(Cl)c1ccc(OCc2ccccc2)nc1 ; CCOC(=O)[C@@H]1[C@@H]2CC[C@H](CN1S(=O)(=O)c1ccc(OCc3ccccc3)nc1)N2C(=O)OCCOC]

To a stirring solution of 2-ethyl 8-(2-methoxyethyl) (1S,2S,5R)-3,8-diazabicyclo[3.2.1]octane-2,8-dicarboxylate (800 mg, 2.79 mmol) in THF (8 mL) under nitrogen at 0-10° C. was added DIPEA (1.47 mL, 8.37 mmol) and stirred for 20 min. 6-(Benzyloxy)-pyridine-3-sulfonyl chloride (1.27 g, 4.18 mmol) in THF (10 mL) was added and the reaction mixture was stirred at 60° C. for 16 h while being monitored by TLC. The reaction was cooled to RT, diluted with water (15 mL) and extracted with ethyl acetate (2×30 mL). The combined organic layer was dried over anhydrous Na₂SO₄, filtered and concentrated under reduced pressure to obtain crude product. The crude product was purified by silica gel column chromatography (100-200 mesh, 25-30% EtOAc in hexanes) to afford 2-ethyl 8-(2-methoxyethyl) (2S,5R)-3-((6-(benzyloxy)pyridin-3-yl)sulfonyl)-3,8-diazabicyclo[3.2.1]octane-2,8-dicarboxylate (820 mg, 1.53 mmol, 55% yield) as a colorless gum. MS (ESI): m/z 534.2 [M+H]⁺.

### Step 2

### Synthesis of (2R,5R)-3-((6-(benzyloxy)pyridin-3-yl)sulfonyl)-8-((2-methoxyethoxy)-carbonyl)-3,8-diazabicyclo[3.2.1]octane-2-carboxylic acid

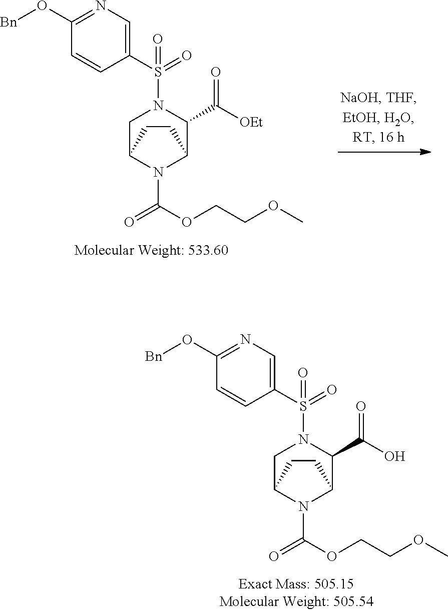 [SMILES: CCOC(=O)[C@@H]1[C@@H]2CC[C@H](CN1S(=O)(=O)c1ccc(OCc3ccccc3)nc1)N2C(=O)OCCOC ; COCCOC(=O)N1[C@@H]2CC[C@H]1[C@H](C(=O)O)N(S(=O)(=O)c1ccc(OCc3ccccc3)nc1)C2]

Similar procedure was followed as described for Step 4 of Example 1, to obtain (2R,5R)-3-((6-(benzyloxy)pyridin-3-yl)sulfonyl)-8-((2-methoxyethoxy)carbonyl)-3,8-diazabicyclo[3.2.1]octane-2-carboxylic acid (600 mg, 1.18 mmol, 77% yield) as a colorless gum. This was used as such for the next step without further purification. MS (ESI): m/z 506.2 [M+H]⁺.

### Step 3

### Synthesis of 2-methoxyethyl (2R,5R)-3-((6-(benzyloxy)pyridin-3-yl)sulfonyl)-2-(((tetrahydro-2H-pyran-2-yl)oxy)carbamoyl)-3,8-diazabicyclo[3.2.1]octane-8-carboxylate

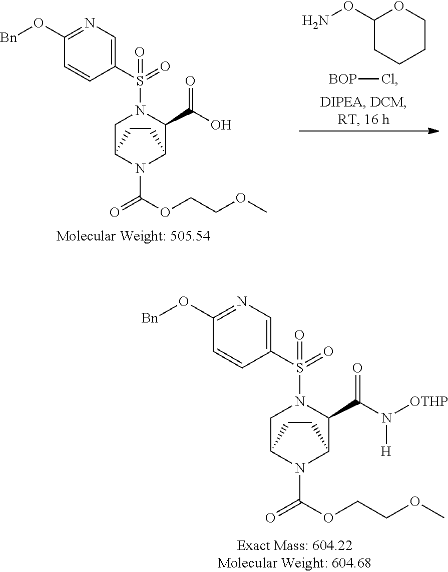

Similar procedure was followed as described for Step 3 of Example 10, to obtain 2-methoxyethyl (2R,5R)-3-((6-(benzyloxy)pyridin-3-yl)sulfonyl)-2-(((tetrahydro-2H-pyran-2-yl)oxy)carbamoyl)-3,8-diazabicyclo[3.2.1]octane-8-carboxylate (600 mg, 0.99 mmol, 84% yield) as a colorless oil. This was used as such for the next step without further purification. MS (ESI): m/z 603.3 [M−H]⁻.

### Step 4

### Synthesis of 2-methoxyethyl (2R,5R)-3-((6-hydroxypyridin-3-yl)sulfonyl)-2-(((tetrahydro-2H-pyran-2-yl)oxy)carbamoyl)-3,8-diazabicyclo[3.2.1]octane-8-carboxylate

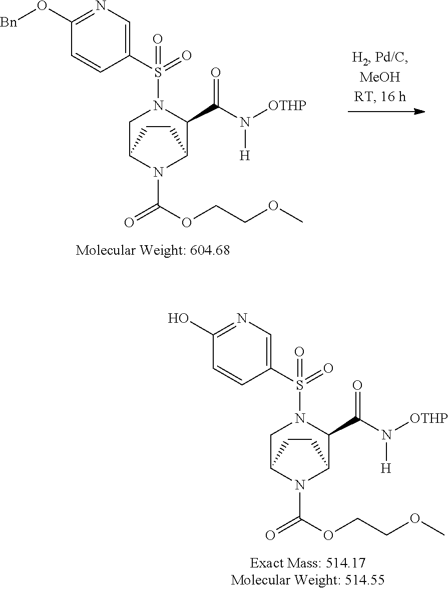

To a stirred solution of 2-methoxyethyl (2R,5R)-3-((6-(benzyloxy)pyridin-3-yl)sulfonyl)-2-(((tetrahydro-2H-pyran-2-yl)oxy)carbamoyl)-3,8-diazabicyclo[3.2.1]octane-8-carboxylate (350 mg, 0.58 mmol) in MeOH (10 mL) was added Pd/C (105 mg, 30% w/w) under N2 atmosphere. The reaction was stirred under H₂ balloon pressure for 16 h while being monitored by TLC. Reaction mixture was filtered through a celite bed and washed with MeOH (20 mL). The filtrate was evaporated under reduced pressure to afford 2-methoxyethyl (2R,5R)-3-((6-hydroxypyridin-3-yl)sulfonyl)-2-(((tetrahydro-2H-pyran-2-yl)oxy)carbamoyl)-3,8-diazabicyclo[3.2.1]octane-8-carboxylate (250 mg, 0.48 mmol, 83% yield) as colorless liquid; MS (ESI): m/z 513.2 [M−H]⁻.

### Step 5

### Synthesis of 2-methoxyethyl (2R,5R)-3-((6-(benzo[d]oxazol-2-yloxy)pyridin-3-yl)sulfonyl)-2-(((tetrahydro-2H-pyran-2-yl)oxy)carbamoyl)-3,8-diazabicyclo[3.2.1]octane-8-carboxylate

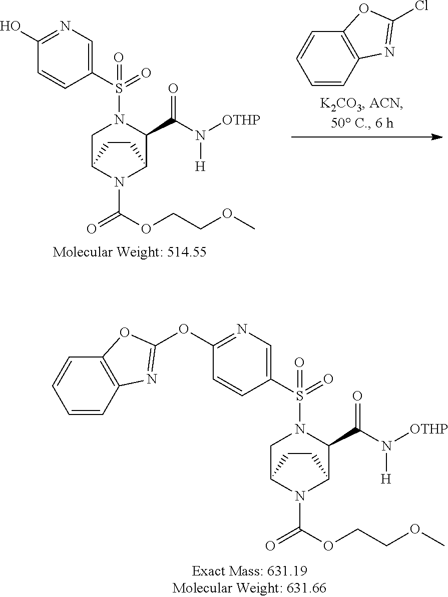

To a stirring solution of 2-methoxyethyl (2R,5R)-3-((6-hydroxypyridin-3-yl)sulfonyl)-2-(((tetrahydro-2H-pyran-2-yl)oxy)carbamoyl)-3,8-diazabicyclo[3.2.1]octane-8-carboxylate (240 mg, 0.46 mmol) in ACN (5 mL) was added K₂CO₃ (192 mg, 1.38 mmol) under nitrogen at 0-10° C. and stirred for 20 min. 2-Chlorobenzo[d]oxazole (79 mg, 0.51 mmol) in ACN (2 mL) was added to the reaction and stirred at 50° C. for 6 h while being monitored by TLC. The reaction was cooled to room temperature, diluted with water (5 mL) and extracted with ethyl acetate (2×20 mL). The combined organic layer was dried over anhydrous Na₂SO₄, filtered and concentrated under reduced pressure. The crude product was purified by silica gel column chromatography (100-200 mesh, 60-80% EtOAc in hexanes) to afford 2-methoxyethyl (2R,5R)-3-((6-(benzo[d]oxazol-2-yloxy)pyridin-3-yl)sulfonyl)-2-(((tetrahydro-2H-pyran-2-yl)oxy)carbamoyl)-3,8-diazabicyclo[3.2.1]octane-8-carboxylate (150 mg, 0.24 mmol, 51% yield) as a colorless gum; MS (ESI): m/z 630.2 [M−H]⁻.

### Step 6

### Synthesis of 2-methoxyethyl (1S,2R,5R)-3-((6-(benzo[d]oxazol-2-yloxy)pyridin-3-yl)sulfonyl)-2-(hydroxycarbamoyl)-3,8-diazabicyclo[3.2.1]octane-8-carboxylate (Example 84)

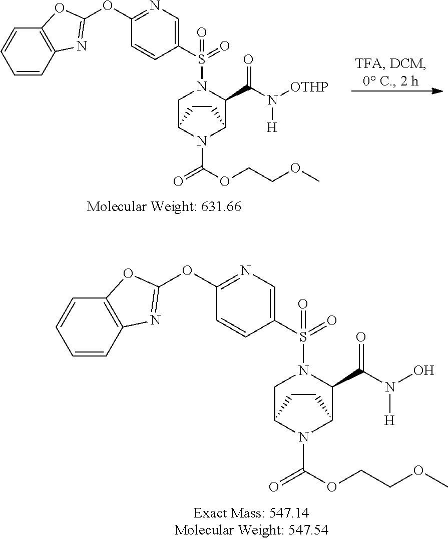

To a stirred solution of 2-methoxyethyl (2R,5R)-3-((6-(benzo[d]oxazol-2-yloxy)pyridin-3-yl)sulfonyl)-2-(((tetrahydro-2H-pyran-2-yl)oxy)carbamoyl)-3,8-diazabicyclo[3.2.1]octane-8-carboxylate (140 mg, 0.22 mmol) in DCM (5 mL) was added trifluoroacetic acid (0.17 mL, 2.2 mmol) dropwise at 0-10° C. and stirred for 2 h while being monitored by TLC. The reaction was quenched with aqueous saturated NaHCO₃ solution (10 mL) and was extracted with DCM (2×20 mL). The organic layer was separated, dried over anhydrous Na₂SO₄, filtered, and concentrated under reduced pressure to afford crude product. The crude compound was purified by Biotage RP purification using 0.1% formic acid in water and ACN, and pure fractions were lyophilized to afford 2-methoxyethyl (1S,2R,5R)-3-((6-(benzo[d]oxazol-2-yloxy)pyridin-3-yl)sulfonyl)-2-(hydroxycarbamoyl)-3,8-diazabicyclo[3.2.1]octane-8-carboxylate (Example 84) (13 mg, 0.023 mmol, 11% yield) as an off-white solid. MS (ESI): m/z 548.2 [M+H]⁺; ¹H NMR (400 MHz, DMSO-d6): δ 10.72 (brs, 1H), 8.81 (brs, 1H), 8.51-8.49 (m, 1H), 7.90-7.85 (m, 2H), 7.76-7.69 (m, 1H), 7.57-7.49 (m, 2H), 6.71 (d, J=10.0 Hz, 1H), 4.80-4.40 (m, 1H), 4.32-4.20 (m, 1H), 4.15-3.80 (m, 3H), 3.74-3.35 (m, 4H), 3.24 (s, 3H), 2.02-1.79 (m, 2H), 1.75-1.50 (m, 2H).

### Matrix Metalloproteinase Inhibition Assays

The inhibitory activities of the compounds of the invention were assessed in 96-well microplate format using MMP1, MMP2, MMP9 and MMP13 colorimetric assay kits and TACE fluorometric assay kit from Enzo Life Sciences, Inc. The inhibitory effect of the compounds of the invention on ADAM10 activity were evaluated in 96-well microplate format using fluorometric assay from AnaSpec, Inc.

The MMP-1 Colorimetric Drug Discovery Kit (catalog #BML-AK404) is a complete assay system designed to screen inhibitors of matrix metalloproteinase-1 using a thiopeptide as a chromogenic substrate (Ac-PLG-[2-mercapto-4-methylpentanoyl]-LG-OC₂H₅). The MMP cleavage site peptide bond is replaced by a thioester bond in the thiopeptide. Hydrolysis of this bond by an MMP produces a sulfhydryl group, which reacts with DTNB [5,5′-dithiobis(2-nitrobenzoic acid), Ellman's reagent] to form 2-nitro-5-thiobenzoic acid, that can be detected spectrophotometrically.

The MMP-2 Colorimetric Drug Discovery Kit (catalog #BML-AK408) is a complete assay system designed to screen MMP-2 inhibitors using a thiopeptide as a chromogenic substrate (Ac-PLG-[2-mercapto-4-methylpentanoyl]-LG-OC₂H₅). The MMP cleavage site peptide bond is replaced by a thioester bond in the thiopeptide. Hydrolysis of this bond by an MMP produces a sulfhydryl group, which reacts with DTNB [5,5′-dithiobis(2-nitrobenzoic acid), Ellman's reagent] to form 2-nitro-5-thiobenzoic acid, that can be detected spectrophotometrically.

The MMP-9 Colorimetric Drug Discovery Kit (catalog #BML-AK410) is a complete assay system designed to screen inhibitors of matrix metalloproteinase-9 using a thiopeptide as a chromogenic substrate (Ac-PLG-[2-mercapto-4-methylpentanoyl]-LG-OC₂H₅). The MMP cleavage site peptide bond is replaced by a thioester bond in the thiopeptide. Hydrolysis of this bond by an MMP produces a sulfhydryl group, which reacts with DTNB [5,5′-dithiobis(2-nitrobenzoic acid), Ellman's reagent] to form 2-nitro-5-thiobenzoic acid, that can be detected spectrophotometrically.

The MMP-13 Colorimetric Drug Discovery Kit (catalog #BML-AK412) is a complete assay system designed to screen inhibitors of matrix metalloproteinase-13 using a thiopeptide as a chromogenic substrate (Ac-PLG-[2-mercapto-4-methylpentanoyl]-LG-OC₂H₅). The MMP cleavage site peptide bond is replaced by a thioester bond in the thiopeptide. Hydrolysis of this bond by an MMP produces a sulfhydryl group, which reacts with DTNB [5,5′-dithiobis(2-nitrobenzoic acid), Ellman's reagent] to form 2-nitro-5-thiobenzoic acid, that can be detected spectrophotometrically.

The TACE Fluorometric Drug Discovery Kit (catalog #BML-AK310) is a complete assay system designed to screen ADAM17 (TACE) inhibitors using a quenched fluorogenic peptide: Mca-PLAQAV-Dpa-RSSSR—NH₂. Mca fluorescence is quenched by the Dpa group until cleavage by proteases separates the two moieties leading to an increase in Mca fluorescence.

SensoLyte® 520 ADAM10 Activity Assay Kit (catalog #AS-72226) is a complete assay system designed to screen ADAM10 inhibitors using FRET-based peptide substrate containing the 5-FAM/QXL™ 520 fluorophore/quencher pair. The fluorescence of 5-FAM is quenched by QXL™ 520 and recovered upon cleavage of the peptide by active ADAM10.

The inhibitory activities of the compounds of the invention against MMP1, MMP2, MMP9 and MMP13 were tested according to the manufacturer's assay protocols and the absorbance at 415 nm was monitored using the Tecan Infinite® 200 Pro F Nano⁺ microplate reader.

The inhibitory activity of the compounds of the invention against TACE was tested according to the manufacturer's assay protocol and the fluorescence was monitored at excitation/emission wavelengths=320 nm/420 nm using Tecan Infinite® 200 Pro F Nano⁺ microplate reader.

The inhibitory activity of the compounds of the invention against ADAM10 was tested according to the manufacturer's assay protocol and the fluorescence was monitored at excitation/emission wavelengths=495 nm/520 nm using Tecan Infinite® 200 Pro F Nano⁺ microplate reader.

The normalized values were analyzed using GraphPadPrism 9 for Windows (GraphPad Software, San Diego, CA, USA) software by plotting inhibition curves and determining the IC₅₀ values (in nM).

The calculated IC₅₀ values were divided into the following classes:

A: <50 nM; B: 50-1000 nM; C: >1000-10,000 nM; D: >10,000 nM

The inhibitory activity classes of the exemplary compounds according to the invention are presented in Table 16.

TABLE 16

| | | MMP | MMP | MMP | MMP | | ADAM |
|---|---|---|---|---|---|---|---|
| Ex. | Structure | 1 | 2 | 9 | 13 | TACE | 10 |
| 1 | 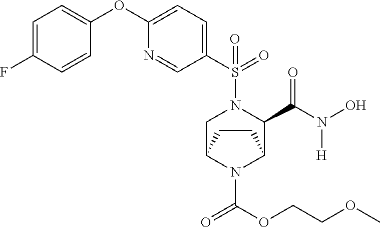 [SMILES: COCCOC(=O)N1[C@@H]2CC[C@H]1[C@H](C(=O)NO)N(S(=O)(=O)c1ccc(Oc3ccc(F)cc3)nc1)C2] | B | A | A | A | C | C |
| 2 | 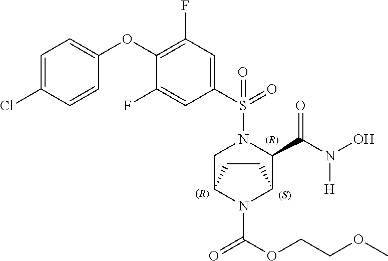 | | B | B | B | | |
| 3 | 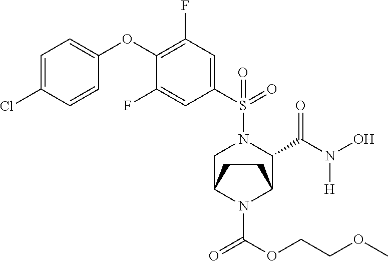 [SMILES: COCCOC(=O)N1[C@H]2CC[C@@H]1[C@@H](C(=O)NO)N(S(=O)(=O)c1cc(F)c(Oc3ccc(Cl)cc3)c(F)c1)C2] | | C | C | C | | |
| 4 | 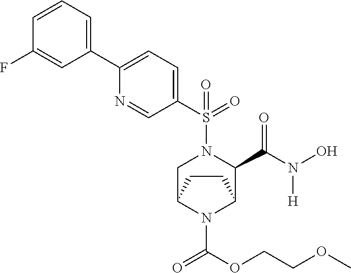 [SMILES: COCCOC(=O)N1[C@@H]2CC[C@H]1[C@H](C(=O)NO)N(S(=O)(=O)c1ccc(-c3cccc(F)c3)nc1)C2] | | B | C | B | | |
| 5 | 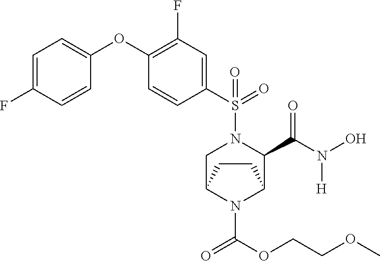 [SMILES: COCCOC(=O)N1[C@@H]2CC[C@H]1[C@H](C(=O)NO)N(S(=O)(=O)c1ccc(Oc3ccc(F)cc3)c(F)c1)C2] | B | A | A | A | B | B |
| 6 | 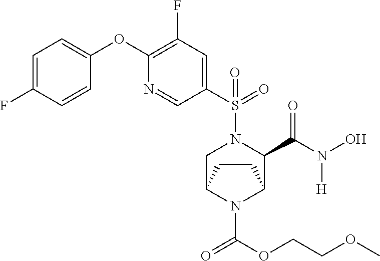 [SMILES: COCCOC(=O)N1[C@@H]2CC[C@H]1[C@H](C(=O)NO)N(S(=O)(=O)c1cnc(Oc3ccc(F)cc3)c(F)c1)C2] | C | A | A | A | C | |
| 7 | 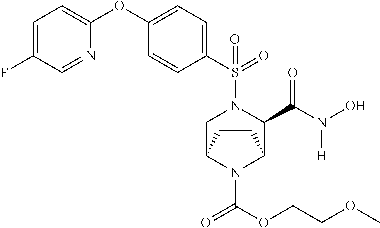 [SMILES: COCCOC(=O)N1[C@@H]2CC[C@H]1[C@H](C(=O)NO)N(S(=O)(=O)c1ccc(Oc3ccc(F)cn3)cc1)C2] | B | A | A | A | B | B |
| 8 | 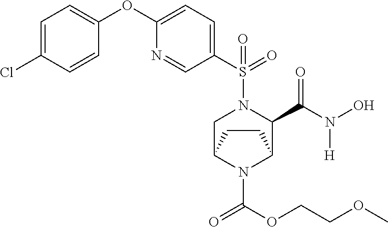 [SMILES: COCCOC(=O)N1[C@@H]2CC[C@H]1[C@H](C(=O)NO)N(S(=O)(=O)c1ccc(Oc3ccc(Cl)cc3)nc1)C2] | B | A | A | A | B | C |
| 9 | 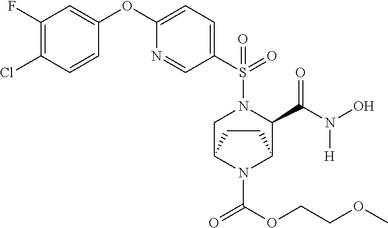 [SMILES: COCCOC(=O)N1[C@@H]2CC[C@H]1[C@H](C(=O)NO)N(S(=O)(=O)c1ccc(Oc3ccc(Cl)c(F)c3)nc1)C2] | B | A | A | A | B | C |
| 10 | 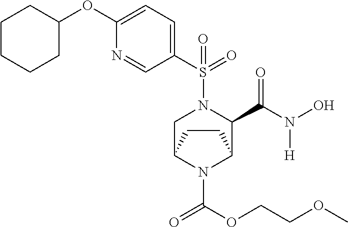 [SMILES: COCCOC(=O)N1[C@@H]2CC[C@H]1[C@H](C(=O)NO)N(S(=O)(=O)c1ccc(OC3CCCCC3)nc1)C2] | B | A | A | A | C | D |
| 11 | 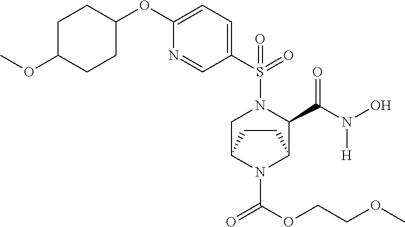 | C | A | A | B | C | |
| 12 | 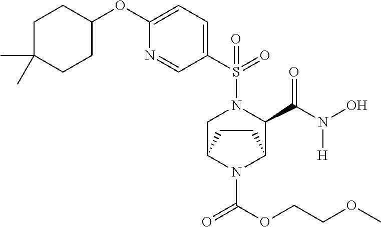 | C | B | A | B | C | |
| 13 | 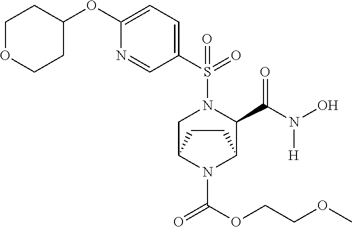 [SMILES: COCCOC(=O)N1[C@@H]2CC[C@H]1[C@H](C(=O)NO)N(S(=O)(=O)c1ccc(OC3CCOCC3)nc1)C2] | C | B | A | A | C | |
| 14 | 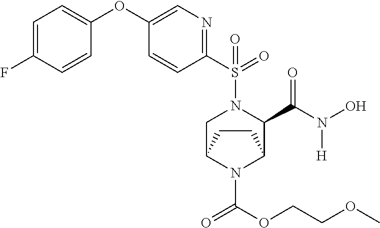 [SMILES: COCCOC(=O)N1[C@@H]2CC[C@H]1[C@H](C(=O)NO)N(S(=O)(=O)c1ccc(Oc3ccc(F)cc3)cn1)C2] | C | A | A | A | C | |
| 15 | 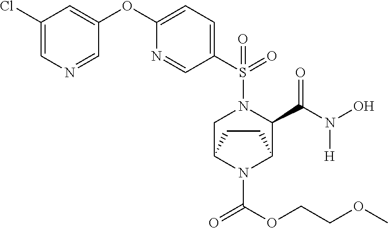 [SMILES: COCCOC(=O)N1[C@@H]2CC[C@H]1[C@H](C(=O)NO)N(S(=O)(=O)c1ccc(Oc3cncc(Cl)c3)nc1)C2] | C | B | B | B | B | |
| 16 | 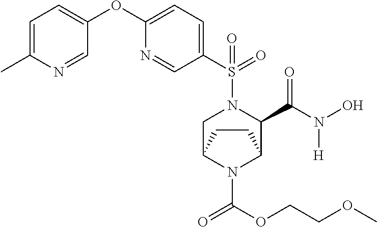 | C | A | B | B | C | |
| 17 | 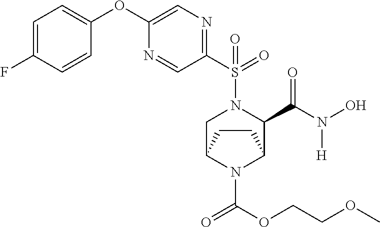 [SMILES: COCCOC(=O)N1[C@@H]2CC[C@H]1[C@H](C(=O)NO)N(S(=O)(=O)c1cnc(Oc3ccc(F)cc3)cn1)C2] | D | C | C | C | D | |
| 18 | 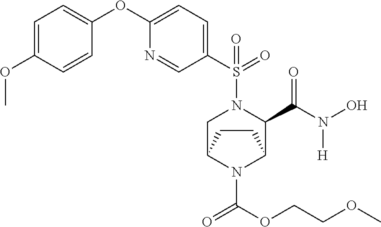 [SMILES: COCCOC(=O)N1[C@@H]2CC[C@H]1[C@H](C(=O)NO)N(S(=O)(=O)c1ccc(Oc3ccc(OC)cc3)nc1)C2] | C | A | A | A | C | C |
| 19 | 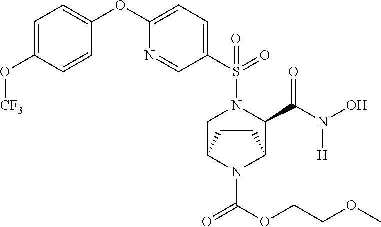 [SMILES: COCCOC(=O)N1[C@@H]2CC[C@H]1[C@H](C(=O)NO)N(S(=O)(=O)c1ccc(Oc3ccc(OC(F)(F)F)cc3)nc1)C2] | C | A | A | A | C | D |
| 20 | 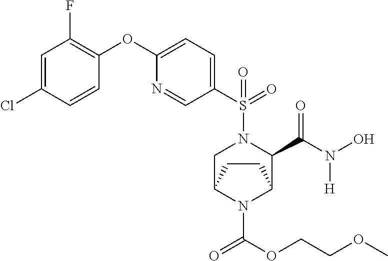 [SMILES: COCCOC(=O)N1[C@@H]2CC[C@H]1[C@H](C(=O)NO)N(S(=O)(=O)c1ccc(Oc3ccc(Cl)cc3F)nc1)C2] | B | A | A | A | B | C |
| 21 | 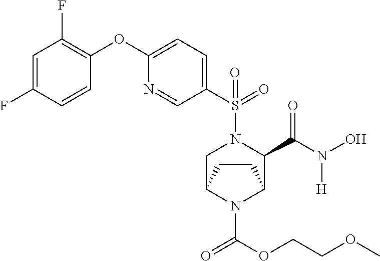 [SMILES: COCCOC(=O)N1[C@@H]2CC[C@H]1[C@H](C(=O)NO)N(S(=O)(=O)c1ccc(Oc3ccc(F)cc3F)nc1)C2] | B | A | A | A | B | D |
| 22 | 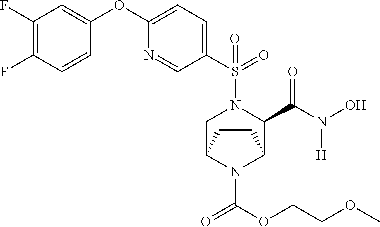 [SMILES: COCCOC(=O)N1[C@@H]2CC[C@H]1[C@H](C(=O)NO)N(S(=O)(=O)c1ccc(Oc3ccc(F)c(F)c3)nc1)C2] | B | A | A | A | B | D |
| 23 | 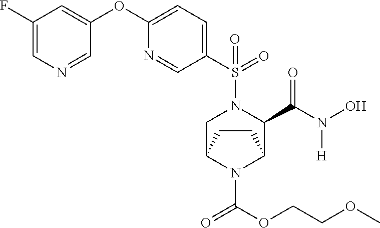 [SMILES: COCCOC(=O)N1[C@@H]2CC[C@H]1[C@H](C(=O)NO)N(S(=O)(=O)c1ccc(Oc3cncc(F)c3)nc1)C2] | B | A | B | B | C | |
| 24 | 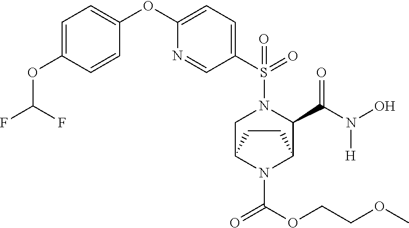 [SMILES: COCCOC(=O)N1[C@@H]2CC[C@H]1[C@H](C(=O)NO)N(S(=O)(=O)c1ccc(Oc3ccc(OC(F)F)cc3)nc1)C2] | C | A | A | A | B | C |
| 25 | 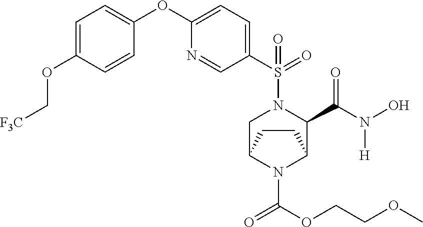 [SMILES: COCCOC(=O)N1[C@@H]2CC[C@H]1[C@H](C(=O)NO)N(S(=O)(=O)c1ccc(Oc3ccc(OCC(F)(F)F)cc3)nc1)C2] | C | A | A | A | D | D |
| 26 | 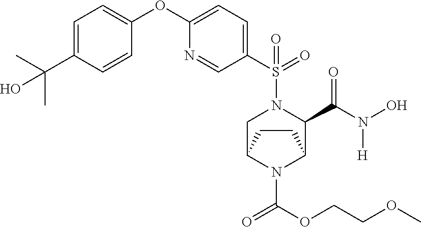 [SMILES: COCCOC(=O)N1[C@@H]2CC[C@H]1[C@H](C(=O)NO)N(S(=O)(=O)c1ccc(Oc3ccc(C(C)(C)O)cc3)nc1)C2] | D | A | B | B | C | |
| 27 | 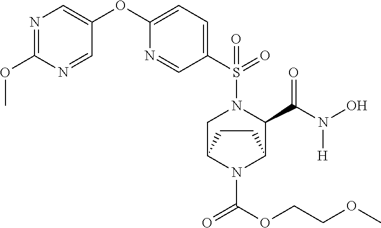 [SMILES: COCCOC(=O)N1[C@@H]2CC[C@H]1[C@H](C(=O)NO)N(S(=O)(=O)c1ccc(Oc3cnc(OC)nc3)nc1)C2] | D | A | B | B | C | |
| 28 | 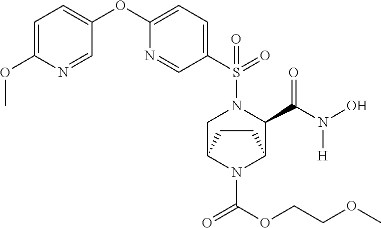 [SMILES: COCCOC(=O)N1[C@@H]2CC[C@H]1[C@H](C(=O)NO)N(S(=O)(=O)c1ccc(Oc3ccc(OC)nc3)nc1)C2] | B | A | A | A | C | D |
| 29 | 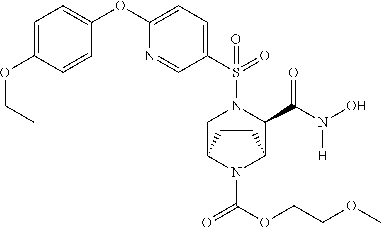 [SMILES: CCOc1ccc(Oc2ccc(S(=O)(=O)N3C[C@H]4CC[C@@H]([C@@H]3C(=O)NO)N4C(=O)OCCOC)cn2)cc1] | B | A | A | A | C | D |
| 30 | 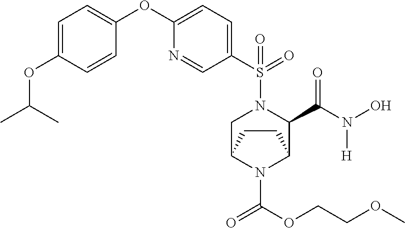 [SMILES: COCCOC(=O)N1[C@@H]2CC[C@H]1[C@H](C(=O)NO)N(S(=O)(=O)c1ccc(Oc3ccc(OC(C)C)cc3)nc1)C2] | B | A | A | A | C | D |
| 31 | 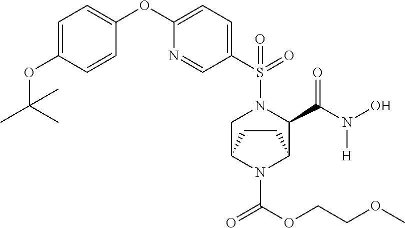 [SMILES: COCCOC(=O)N1[C@@H]2CC[C@H]1[C@H](C(=O)NO)N(S(=O)(=O)c1ccc(Oc3ccc(OC(C)(C)C)cc3)nc1)C2] | D | A | A | A | D | D |
| 32 | 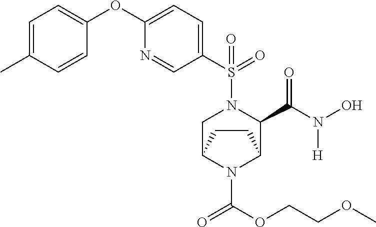 | B | A | A | A | C | D |
| 33 | 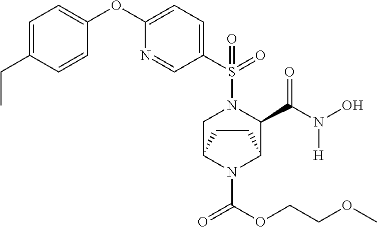 [SMILES: CCc1ccc(Oc2ccc(S(=O)(=O)N3C[C@H]4CC[C@@H]([C@@H]3C(=O)NO)N4C(=O)OCCOC)cn2)cc1] | C | A | A | A | C | D |
| 34 | 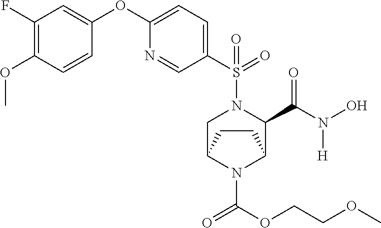 [SMILES: COCCOC(=O)N1[C@@H]2CC[C@H]1[C@H](C(=O)NO)N(S(=O)(=O)c1ccc(Oc3ccc(OC)c(F)c3)nc1)C2] | C | A | A | A | C | C |
| 35 | 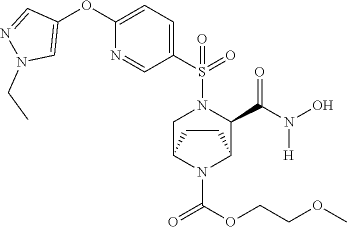 [SMILES: CCn1cc(Oc2ccc(S(=O)(=O)N3C[C@H]4CC[C@@H]([C@@H]3C(=O)NO)N4C(=O)OCCOC)cn2)cn1] | B | A | A | A | B | |
| 36 | 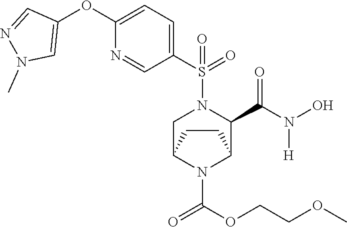 [SMILES: COCCOC(=O)N1[C@@H]2CC[C@H]1[C@H](C(=O)NO)N(S(=O)(=O)c1ccc(Oc3cnn(C)c3)nc1)C2] | A | A | A | A | B | C |
| 37 | 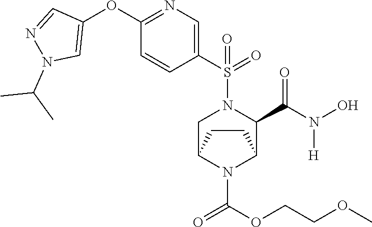 [SMILES: COCCOC(=O)N1[C@@H]2CC[C@H]1[C@H](C(=O)NO)N(S(=O)(=O)c1ccc(Oc3cnn(C(C)C)c3)nc1)C2] | C | B | B | B | B | |
| 38 | 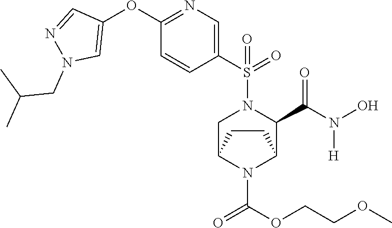 [SMILES: COCCOC(=O)N1[C@@H]2CC[C@H]1[C@H](C(=O)NO)N(S(=O)(=O)c1ccc(Oc3cnn(CC(C)C)c3)nc1)C2] | C | B | B | B | B | |
| 39 | 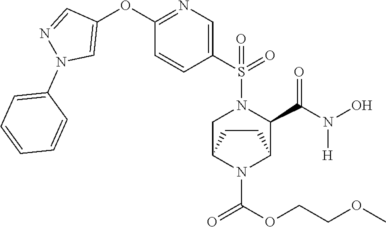 [SMILES: COCCOC(=O)N1[C@@H]2CC[C@H]1[C@H](C(=O)NO)N(S(=O)(=O)c1ccc(Oc3cnn(-c4ccccc4)c3)nc1)C2] | B | A | A | A | B | D |
| 40 | 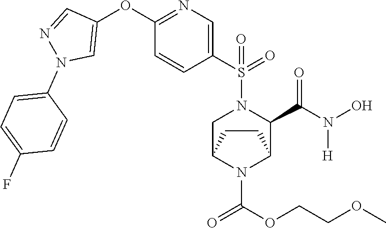 [SMILES: COCCOC(=O)N1[C@@H]2CC[C@H]1[C@H](C(=O)NO)N(S(=O)(=O)c1ccc(Oc3cnn(-c4ccc(F)cc4)c3)nc1)C2] | B | A | A | A | B | |
| 41 | 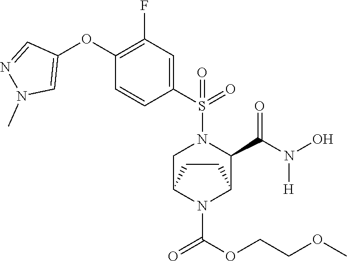 [SMILES: COCCOC(=O)N1[C@@H]2CC[C@H]1[C@H](C(=O)NO)N(S(=O)(=O)c1ccc(Oc3cnn(C)c3)c(F)c1)C2] | A | A | A | A | B | B |
| 42 | 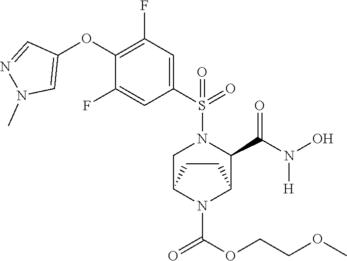 [SMILES: COCCOC(=O)N1[C@@H]2CC[C@H]1[C@H](C(=O)NO)N(S(=O)(=O)c1cc(F)c(Oc3cnn(C)c3)c(F)c1)C2] | C | B | B | B | B | |
| 43 | 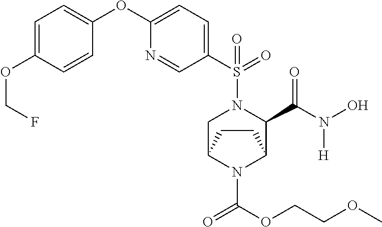 [SMILES: COCCOC(=O)N1[C@@H]2CC[C@H]1[C@H](C(=O)NO)N(S(=O)(=O)c1ccc(Oc3ccc(OCF)cc3)nc1)C2] | C | A | A | A | C | |
| 44 | 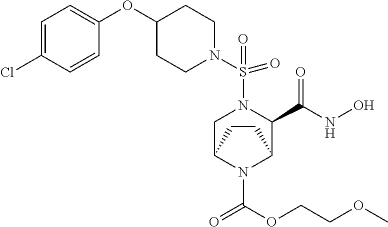 [SMILES: COCCOC(=O)N1[C@@H]2CC[C@H]1[C@H](C(=O)NO)N(S(=O)(=O)N1CCC(Oc3ccc(Cl)cc3)CC1)C2] | B | A | A | A | A | B |
| 45 | 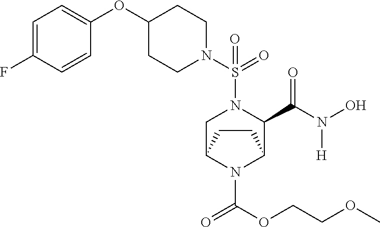 [SMILES: COCCOC(=O)N1[C@@H]2CC[C@H]1[C@H](C(=O)NO)N(S(=O)(=O)N1CCC(Oc3ccc(F)cc3)CC1)C2] | B | A | A | A | B | C |
| 46 | 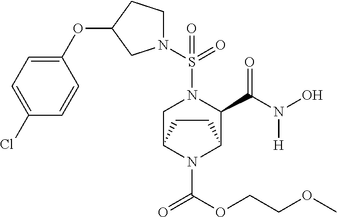 [SMILES: COCCOC(=O)N1[C@@H]2CC[C@H]1[C@H](C(=O)NO)N(S(=O)(=O)N1CCC(Oc3ccc(Cl)cc3)C1)C2] | D | B | B | B | B | |
| 47 | 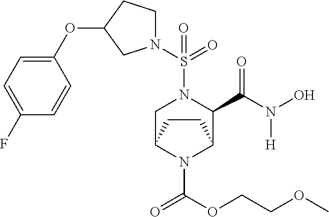 [SMILES: COCCOC(=O)N1[C@@H]2CC[C@H]1[C@H](C(=O)NO)N(S(=O)(=O)N1CCC(Oc3ccc(F)cc3)C1)C2] | D | C | C | C | B | |
| 48 | 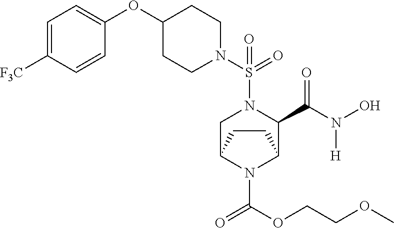 [SMILES: COCCOC(=O)N1[C@@H]2CC[C@H]1[C@H](C(=O)NO)N(S(=O)(=O)N1CCC(Oc3ccc(C(F)(F)F)cc3)CC1)C2] | C | A | A | A | A | B |
| 49 | 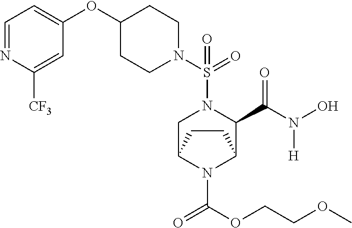 [SMILES: COCCOC(=O)N1[C@@H]2CC[C@H]1[C@H](C(=O)NO)N(S(=O)(=O)N1CCC(Oc3ccnc(C(F)(F)F)c3)CC1)C2] | D | B | B | B | C | |
| 50 | 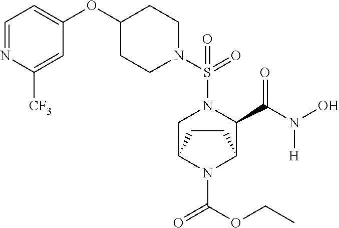 [SMILES: CCOC(=O)N1[C@@H]2CC[C@H]1[C@H](C(=O)NO)N(S(=O)(=O)N1CCC(Oc3ccnc(C(F)(F)F)c3)CC1)C2] | D | B | B | B | C | |
| 51 | 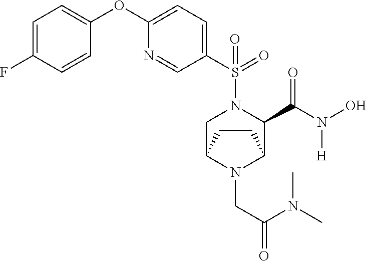 [SMILES: CN(C)C(=O)CN1[C@@H]2CC[C@H]1[C@H](C(=O)NO)N(S(=O)(=O)c1ccc(Oc3ccc(F)cc3)nc1)C2] | C | A | B | A | D | |
| 52 | 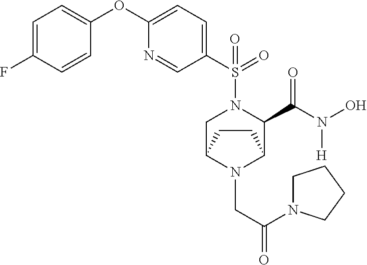 [SMILES: O=C(NO)[C@H]1[C@@H]2CC[C@H](CN1S(=O)(=O)c1ccc(Oc3ccc(F)cc3)nc1)N2CC(=O)N1CCCC1] | C | A | B | A | D | |
| 53 | 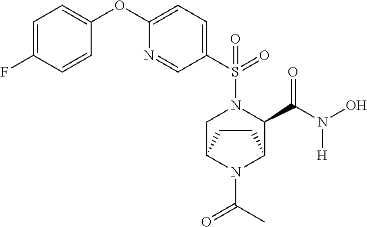 [SMILES: CC(=O)N1[C@@H]2CC[C@H]1[C@H](C(=O)NO)N(S(=O)(=O)c1ccc(Oc3ccc(F)cc3)nc1)C2] | B | A | A | A | C | D |
| 54 | 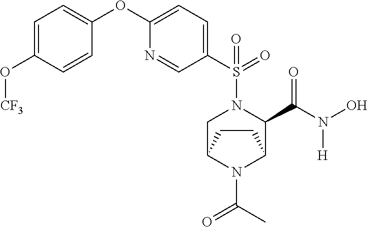 [SMILES: CC(=O)N1[C@@H]2CC[C@H]1[C@H](C(=O)NO)N(S(=O)(=O)c1ccc(Oc3ccc(OC(F)(F)F)cc3)nc1)C2] | C | A | A | A | C | D |
| 55 | 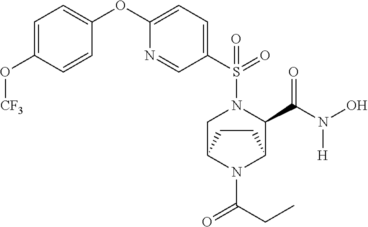 [SMILES: CCC(=O)N1[C@@H]2CC[C@H]1[C@H](C(=O)NO)N(S(=O)(=O)c1ccc(Oc3ccc(OC(F)(F)F)cc3)nc1)C2] | C | A | A | A | C | D |
| 56 | 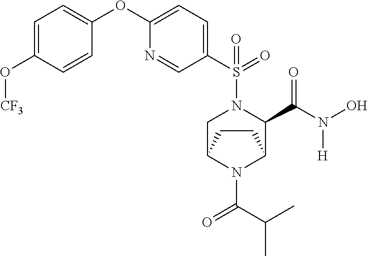 [SMILES: CC(C)C(=O)N1[C@@H]2CC[C@H]1[C@H](C(=O)NO)N(S(=O)(=O)c1ccc(Oc3ccc(OC(F)(F)F)cc3)nc1)C2] | D | A | A | A | C | D |
| 57 | 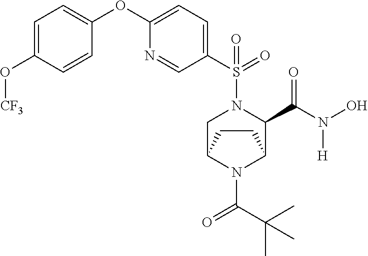 [SMILES: CC(C)(C)C(=O)N1[C@@H]2CC[C@H]1[C@H](C(=O)NO)N(S(=O)(=O)c1ccc(Oc3ccc(OC(F)(F)F)cc3)nc1)C2] | C | A | A | A | C | D |
| 58 | 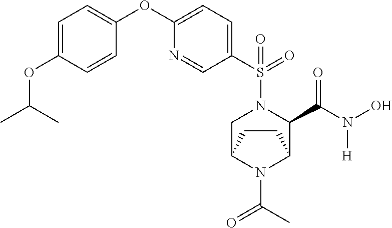 [SMILES: CC(=O)N1[C@@H]2CC[C@H]1[C@H](C(=O)NO)N(S(=O)(=O)c1ccc(Oc3ccc(OC(C)C)cc3)nc1)C2] | C | A | A | A | D | D |
| 59 | 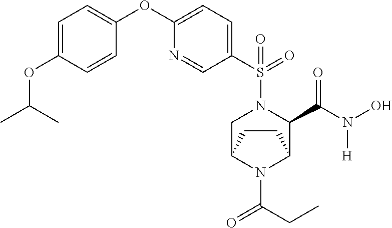 [SMILES: CCC(=O)N1[C@@H]2CC[C@H]1[C@H](C(=O)NO)N(S(=O)(=O)c1ccc(Oc3ccc(OC(C)C)cc3)nc1)C2] | C | A | A | A | D | D |
| 60 | 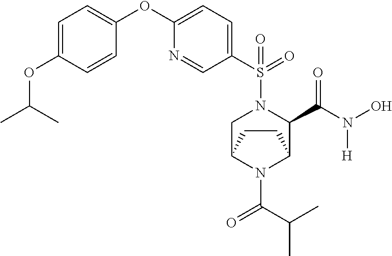 [SMILES: CC(C)Oc1ccc(Oc2ccc(S(=O)(=O)N3C[C@H]4CC[C@@H]([C@@H]3C(=O)NO)N4C(=O)C(C)C)cn2)cc1] | C | A | A | A | D | |
| 61 | 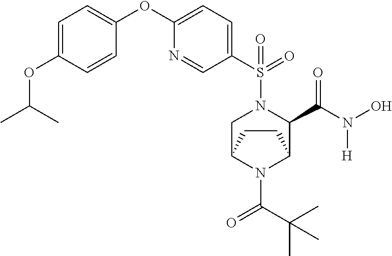 [SMILES: CC(C)Oc1ccc(Oc2ccc(S(=O)(=O)N3C[C@H]4CC[C@@H]([C@@H]3C(=O)NO)N4C(=O)C(C)(C)C)cn2)cc1] | C | A | B | A | D | |
| 62 | 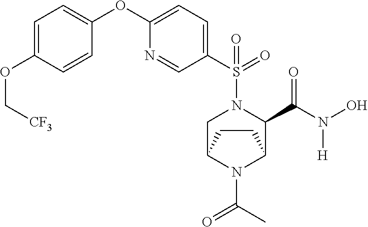 [SMILES: CC(=O)N1[C@@H]2CC[C@H]1[C@H](C(=O)NO)N(S(=O)(=O)c1ccc(Oc3ccc(OCC(F)(F)F)cc3)nc1)C2] | C | A | A | A | D | D |
| 63 | 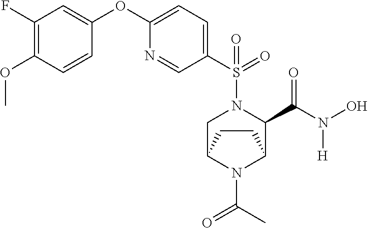 [SMILES: COc1ccc(Oc2ccc(S(=O)(=O)N3C[C@H]4CC[C@@H]([C@@H]3C(=O)NO)N4C(C)=O)cn2)cc1F] | C | A | A | A | C | C |
| 64 | 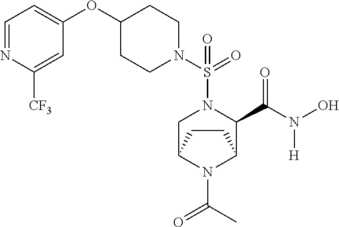 [SMILES: CC(=O)N1[C@@H]2CC[C@H]1[C@H](C(=O)NO)N(S(=O)(=O)N1CCC(Oc3ccnc(C(F)(F)F)c3)CC1)C2] | C | B | B | B | C | |
| 65 | 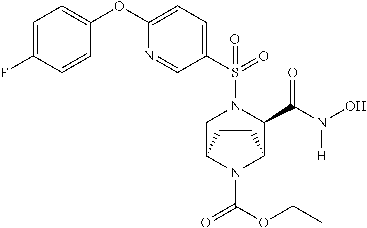 [SMILES: CCOC(=O)N1[C@@H]2CC[C@H]1[C@H](C(=O)NO)N(S(=O)(=O)c1ccc(Oc3ccc(F)cc3)nc1)C2] | B | A | A | A | C | D |
| 66 | 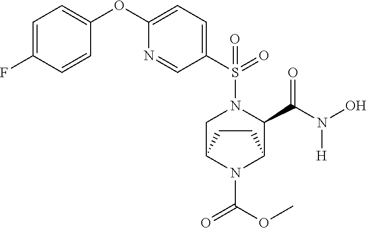 [SMILES: COC(=O)N1[C@@H]2CC[C@H]1[C@H](C(=O)NO)N(S(=O)(=O)c1ccc(Oc3ccc(F)cc3)nc1)C2] | C | A | A | A | C | D |
| 67 | 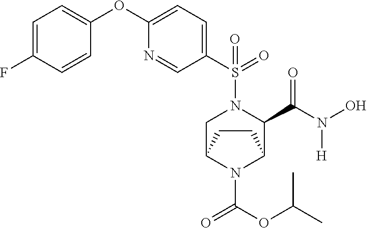 [SMILES: CC(C)OC(=O)N1[C@@H]2CC[C@H]1[C@H](C(=O)NO)N(S(=O)(=O)c1ccc(Oc3ccc(F)cc3)nc1)C2] | B | A | A | A | C | D |
| 68 | 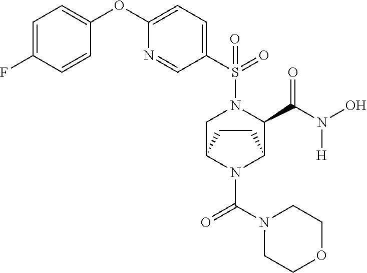 [SMILES: O=C(NO)[C@H]1[C@@H]2CC[C@H](CN1S(=O)(=O)c1ccc(Oc3ccc(F)cc3)nc1)N2C(=O)N1CCOCC1] | C | A | B | A | C | |
| 69 | 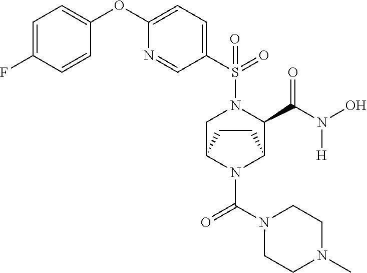 [SMILES: CN1CCN(C(=O)N2[C@@H]3CC[C@H]2[C@H](C(=O)NO)N(S(=O)(=O)c2ccc(Oc4ccc(F)cc4)nc2)C3)CC1] | C | A | B | B | C | |
| 70 | 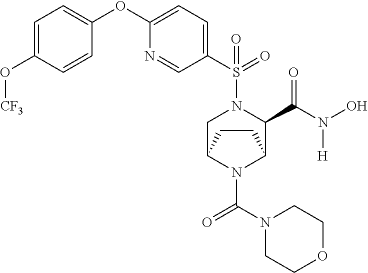 [SMILES: O=C(NO)[C@H]1[C@@H]2CC[C@H](CN1S(=O)(=O)c1ccc(Oc3ccc(OC(F)(F)F)cc3)nc1)N2C(=O)N1CCOCC1] | D | A | A | A | C | D |
| 71 | 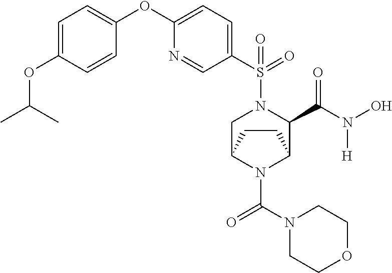 [SMILES: CC(C)Oc1ccc(Oc2ccc(S(=O)(=O)N3C[C@H]4CC[C@@H]([C@@H]3C(=O)NO)N4C(=O)N3CCOCC3)cn2)cc1] | C | A | B | A | D | |
| 72 | 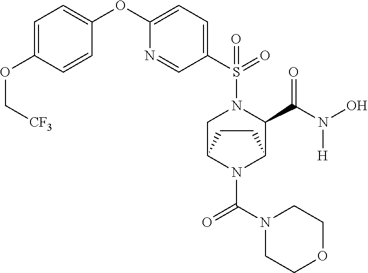 [SMILES: O=C(NO)[C@H]1[C@@H]2CC[C@H](CN1S(=O)(=O)c1ccc(Oc3ccc(OCC(F)(F)F)cc3)nc1)N2C(=O)N1CCOCC1] | C | A | A | A | D | D |
| 73 | 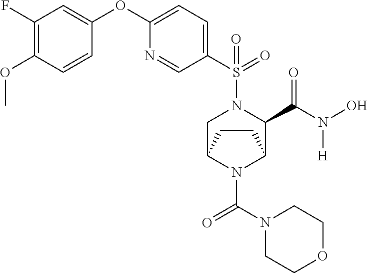 [SMILES: COc1ccc(Oc2ccc(S(=O)(=O)N3C[C@H]4CC[C@@H]([C@@H]3C(=O)NO)N4C(=O)N3CCOCC3)cn2)cc1F] | D | A | A | A | C | C |
| 74 | 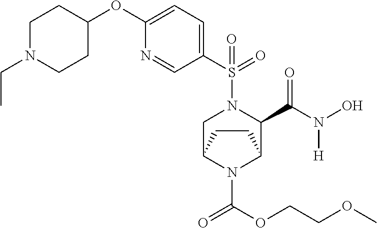 [SMILES: CCN1CCC(Oc2ccc(S(=O)(=O)N3C[C@H]4CC[C@@H]([C@@H]3C(=O)NO)N4C(=O)OCCOC)cn2)CC1] | D | D | C | D | D | |
| 75 | 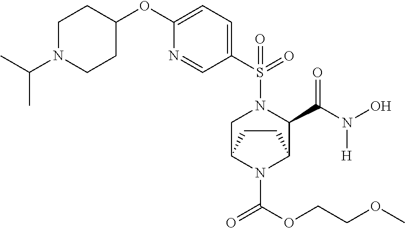 [SMILES: COCCOC(=O)N1[C@@H]2CC[C@H]1[C@H](C(=O)NO)N(S(=O)(=O)c1ccc(OC3CCN(C(C)C)CC3)nc1)C2] | C | B | B | B | C | |
| 76 | 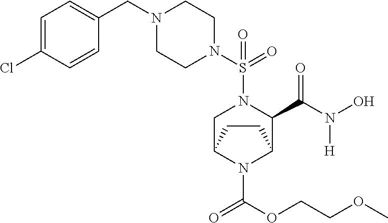 [SMILES: COCCOC(=O)N1[C@@H]2CC[C@H]1[C@H](C(=O)NO)N(S(=O)(=O)N1CCN(Cc3ccc(Cl)cc3)CC1)C2] | D | B | B | B | C | |
| 77 | 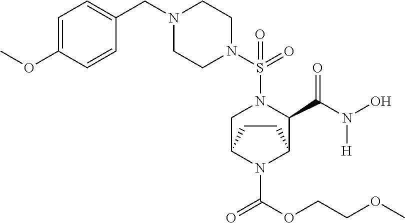 | D | B | B | C | C | |
| 78 | 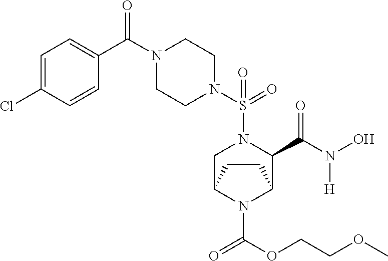 [SMILES: COCCOC(=O)N1[C@@H]2CC[C@H]1[C@H](C(=O)NO)N(S(=O)(=O)N1CCN(C(=O)c3ccc(Cl)cc3)CC1)C2] | D | C | C | C | C | |
| 79 | 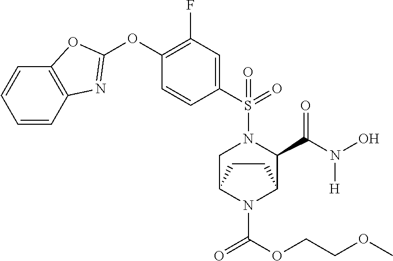 [SMILES: COCCOC(=O)N1[C@@H]2CC[C@H]1[C@H](C(=O)NO)N(S(=O)(=O)c1ccc(Oc3nc4ccccc4o3)c(F)c1)C2] | D | B | C | B | C | |
| 80 | 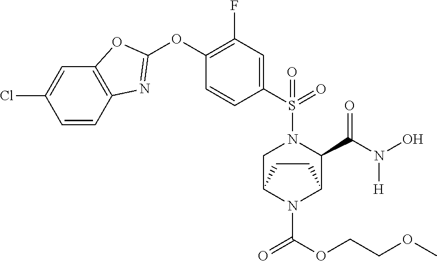 [SMILES: COCCOC(=O)N1[C@@H]2CC[C@H]1[C@H](C(=O)NO)N(S(=O)(=O)c1ccc(Oc3nc4ccc(Cl)cc4o3)c(F)c1)C2] | D | C | C | C | C | |
| 81 | 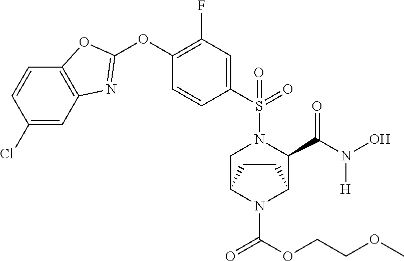 [SMILES: COCCOC(=O)N1[C@@H]2CC[C@H]1[C@H](C(=O)NO)N(S(=O)(=O)c1ccc(Oc3nc4cc(Cl)ccc4o3)c(F)c1)C2] | D | D | C | D | C | |
| 82 | 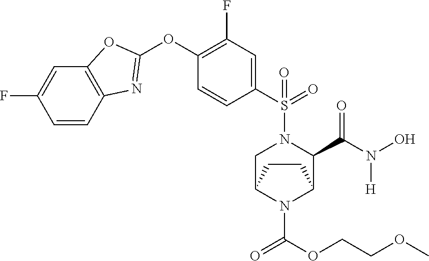 [SMILES: COCCOC(=O)N1[C@@H]2CC[C@H]1[C@H](C(=O)NO)N(S(=O)(=O)c1ccc(Oc3nc4ccc(F)cc4o3)c(F)c1)C2] | D | C | C | C | B | |
| 83 | 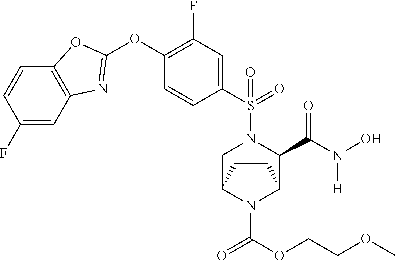 [SMILES: COCCOC(=O)N1[C@@H]2CC[C@H]1[C@H](C(=O)NO)N(S(=O)(=O)c1ccc(Oc3nc4cc(F)ccc4o3)c(F)c1)C2] | | C | C | C | | |
| 84 | 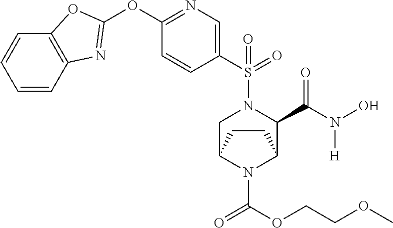 [SMILES: COCCOC(=O)N1[C@@H]2CC[C@H]1[C@H](C(=O)NO)N(S(=O)(=O)c1ccc(Oc3nc4ccccc4o3)nc1)C2] | | D | D | D | | |

1-36. (canceled)

37. A compound of Formula (III): 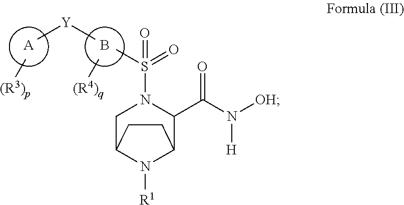
wherein:
\textcircled{A} is phenyl, C₃-C₁₀cycloalkyl, C₂-C₉heterocycloalkyl, or C₂-C₉heteroaryl;
\textcircled{B} is phenyl, C₃-C₁₀cycloalkyl, C₂-C₉heterocycloalkyl, or C₂-C₉heteroaryl;
Y is a direct bond, —O—, —CH₂O—, —OCH₂—, —CH₂—, —C(═O)—, —C(═O)NH—, or —N(R⁵)—;
wherein Y, if asymmetrical, is written in the sequence corresponding to 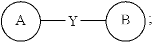
wherein if nitrogen is the attachment point on \textcircled{B} for attaching Y then Y cannot be neither —O—, —CH₂O—, —C(═O)NH—, nor —N(RS)—;
R¹ is hydrogen, —C₁-C₆alkyl, —C₁-C₆haloalkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂;
each R³ and each R⁴ are independently selected from halogen, —C₁-C₆alkyl, —C₁-C₆haloalkyl, —C₁-C₆alkylene-OR⁶, C₁-C₆haloalkoxy, —OR⁶, —N(R⁶)₂, —CN, —C(═O)R⁷, —C(═O)OR⁶, —C(═O)N(R⁶)₂, —NR⁶C(═O)R⁷, —NR⁶SO₂R⁷, —SO₂R⁷, —SO₂N(R⁶)₂, or phenyl optionally substituted with halogen;
R⁵ is hydrogen or —C₁-C₆alkyl;
each R⁶ is independently selected from hydrogen, —C₁-C₆alkyl, —C₁-C₆haloalkyl, —C₁-C₆alkylene-OR⁹, —C₁-C₆alkylene-N(R⁹)₂, and —C₁-C₆alkylene-C₂-C₉heterocycloalkyl;
or —N(R⁶)₂ represents C₃-C₅heterocycloalkyl;
each R⁷ is independently selected from —C₁-C₆alkyl, —C₁-C₆alkylene-OR⁹, —C₁-C₆alkylene-N(R⁹)₂, —C₂-C₉heterocycloalkyl, —C₂-C₉heteroaryl, —C₁-C₆alkylene-C₂-C₉heterocycloalkyl, and —C₁-C₆alkylene-C₂-C₉heteroaryl;
each R⁹ is independently selected from hydrogen and —C₁-C₆alkyl;
each occurrence of C₂-C₉heterocycloalkyl and C₂-C₉heteroaryl being optionally substituted with —C₁-C₆alkyl, halo-C₁-C₆alkyl or halogen;
p is 0, 1, 2, or 3; and
q is 0, 1, 2, or 3;
or a pharmaceutically acceptable salt or solvate thereof.

38. The compound of claim 1, having the structure of Formula (IIIa): 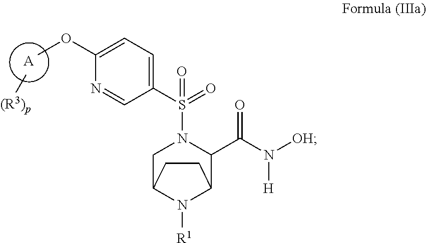
wherein:
\textcircled{A} is phenyl, C₃-C₁₀cycloalkyl, C₂-C₉heterocycloalkyl, or C₂-C₉heteroaryl;
R¹ is hydrogen, —C₁-C₆alkyl, —C₁-C₆haloalkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂;
each R³ is independently selected from halogen, —C₁-C₆alkyl, —C₁-C₆haloalkyl, —C₁-C₆alkylene-OR⁶, C₁-C₆haloalkoxy, —OR⁶, —N(R⁶)₂, —CN, —C(═O)R⁷, —C(═O)OR⁶, —C(═O)N(R⁶)₂, —NR⁶C(═O)R⁷, —NR⁶SO₂R⁷, —SO₂R⁷, —SO₂N(R⁶)₂, or phenyl optionally substituted with halogen;
each R⁶ is independently selected from hydrogen, —C₁-C₆alkyl, —C₁-C₆haloalkyl, —C₁-C₆alkylene-OR⁹, —C₁-C₆alkylene-N(R⁹)₂, and —C₁-C₆alkylene-C₂-C₉heterocycloalkyl;
or —N(R⁶)₂ represents C₃-C₅heterocycloalkyl;
each R⁷ is independently selected from —C₁-C₆alkyl, —C₁-C₆alkylene-OR⁹, —C₁-C₆alkylene-N(R⁹)₂, —C₂-C₉heterocycloalkyl, —C₂-C₉heteroaryl, —C₁-C₆alkylene-C₂-C₉heterocycloalkyl, and —C₁-C₆alkylene-C₂-C₉heteroaryl;
each R⁹ is independently selected from hydrogen and —C₁-C₆alkyl;
each occurrence of C₂-C₉heterocycloalkyl and C₂-C₉heteroaryl being optionally substituted with —C₁-C₆alkyl, halo-C₁-C₆alkyl or halogen; and
p is 0, 1, 2, or 3;
or a pharmaceutically acceptable salt or solvate thereof.

39. The compound of claim 37, having the structure of Formula (IIIb): 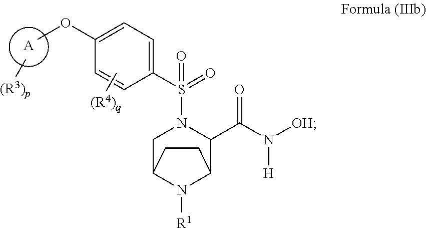
wherein:
\textcircled{A} is phenyl, C₃-C₁₀cycloalkyl, C₂-C₉heterocycloalkyl, or C₂-C₉heteroaryl;
R¹ is hydrogen, —C₁-C₆alkyl, —C₁-C₆haloalkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂;
each R³ and each R⁴ are independently selected from halogen, —C₁-C₆alkyl, —C₁-C₆haloalkyl, —C₁-C₆alkylene-OR⁶, C₁-C₆haloalkoxy, —OR⁶, —N(R⁶)₂, —CN, —C(═O)R⁷, —C(═O)OR⁶, —C(═O)N(R⁶)₂, —NR⁶C(═O)R⁷, —NR⁶SO₂R⁷, —SO₂R⁷, —SO₂N(R⁶)₂, or phenyl optionally substituted with halogen;
each R⁶ is independently selected from hydrogen, —C₁-C₆alkyl, —C₁-C₆haloalkyl, —C₁-C₆alkylene-OR⁹, —C₁-C₆alkylene-N(R⁹)₂, and —C₁-C₆alkylene-C₂-C₉heterocycloalkyl;
or —N(R⁶)₂ represents C₃-C₅heterocycloalkyl;
each R⁷ is independently selected from —C₁-C₆alkyl, —C₁-C₆alkylene-OR⁹, —C₁-C₆alkylene-N(R⁹)₂, —C₂-C₉heterocycloalkyl, —C₂-C₉heteroaryl, —C₁-C₆alkylene-C₂-C₉heterocycloalkyl, and —C₁-C₆alkylene-C₂-C₉heteroaryl;
each R⁹ is independently selected from hydrogen and —C₁-C₆alkyl;
each occurrence of C₂-C₉heterocycloalkyl and C₂-C₉heteroaryl being optionally substituted with —C₁-C₆alkyl, halo-C₁-C₆alkyl or halogen;
p is 0, 1, 2, or 3; and
q is 0, 1, 2, or 3;
or a pharmaceutically acceptable salt or solvate thereof.

40. The compound of claim 37, having the structure of Formula (IIIc): 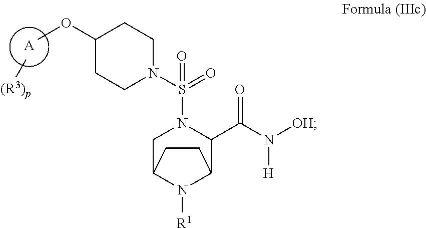
wherein:
\textcircled{A} is phenyl, C₃-C₁₀cycloalkyl, C₂-C₉heterocycloalkyl, or C₂-C₉heteroaryl;
R¹ is hydrogen, —C₁-C₆alkyl, —C₁-C₆haloalkyl, —C₁-C₆alkylene-OR⁶, —C₁-C₆alkylene-N(R⁶)₂, —C(═O)R⁷, —C(═O)OR⁶, —C(═O)N(R⁶)₂, —S(═O)₂R⁷, or —S(═O)₂N(R⁶)₂;
each R³ is independently selected from halogen, —C₁-C₆alkyl, —C₁-C₆haloalkyl, —C₁-C₆alkylene-OR⁶, C₁-C₆haloalkoxy, —OR⁶, —N(R⁶)₂, —CN, —C(═O)R⁷, —C(═O)OR⁶, —C(═O)N(R⁶)₂, —NR⁶C(═O)R⁷, —NR⁶SO₂R⁷, —SO₂R⁷, —SO₂N(R⁶)₂, or phenyl optionally substituted with halogen;
each R⁶ is independently selected from hydrogen, —C₁-C₆alkyl, —C₁-C₆haloalkyl, —C₁-C₆alkylene-OR⁹, —C₁-C₆alkylene-N(R⁹)₂, and —C₁-C₆alkylene-C₂-C₉heterocycloalkyl;
or —N(R⁶)₂ represents C₃-C₅heterocycloalkyl;
each R⁷ is independently selected from —C₁-C₆alkyl, —C₁-C₆alkylene-OR⁹, —C₁-C₆alkylene-N(R⁹)₂, —C₂-C₉heterocycloalkyl, —C₂-C₉heteroaryl, —C₁-C₆alkylene-C₂-C₉heterocycloalkyl, and —C₁-C₆alkylene-C₂-C₉heteroaryl;
each R⁹ is independently selected from hydrogen and —C₁-C₆alkyl;
each occurrence of C₂-C₉heterocycloalkyl and C₂-C₉heteroaryl being optionally substituted with —C₁-C₆alkyl, halo-C₁-C₆alkyl or halogen; and
p is 0, 1, 2, or 3;
or a pharmaceutically acceptable salt or solvate thereof.

41. The compound of claim 37, or a pharmaceutically acceptable salt or solvate thereof, wherein each R³ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂.

42. The compound of claim 37, or a pharmaceutically acceptable salt or solvate thereof, wherein each R³ is independently selected from halogen, —C₁-C₆alkyl, —C₁-C₆haloalkyl, —OR⁶, and —C(═O)OR⁶.

43. The compound of claim 37, or a pharmaceutically acceptable salt or solvate thereof, wherein each R³ is independently selected from halogen and —C₁-C₆alkyl.

44. The compound of claim 37, or a pharmaceutically acceptable salt or solvate thereof, wherein p is 1.

45. The compound of claim 37, or a pharmaceutically acceptable salt or solvate thereof, wherein p is 2.

46. The compound of claim 37, or a pharmaceutically acceptable salt or solvate thereof, wherein p is 0.

47. The compound of claim 37, or a pharmaceutically acceptable salt or solvate thereof, wherein each R⁴ is independently selected from halogen, —C₁-C₆alkyl, —C₁-C₆haloalkyl, —OR⁶, —N(R⁶)₂, —CN, —C(═O)OR⁶, —C(═O)N(R⁶)₂, —SO₂R⁷, and —SO₂N(R⁶)₂.

48. The compound of claim 37, or a pharmaceutically acceptable salt or solvate thereof, wherein each R⁴ is independently selected from halogen, —C₁-C₆alkyl, —C₁-C₆haloalkyl, —OR⁶, and —C(═O)OR⁶.

49. The compound of claim 37, or a pharmaceutically acceptable salt or solvate thereof, wherein each R⁴ is independently selected from halogen and —C₁-C₆alkyl.

50. The compound of claim 37, or a pharmaceutically acceptable salt or solvate thereof, wherein q is 1.

51. The compound of claim 37, or a pharmaceutically acceptable salt or solvate thereof, wherein q is 2.

52. The compound of claim 37, or a pharmaceutically acceptable salt or solvate thereof, wherein q is 0.

53. The compound of claim 37, or a pharmaceutically acceptable salt or solvate thereof, wherein R¹ is —C(═O)OR⁶.

54. The compound of claim 37, that is selected from the following compounds:

| Structure | Name |
|---|---|
| 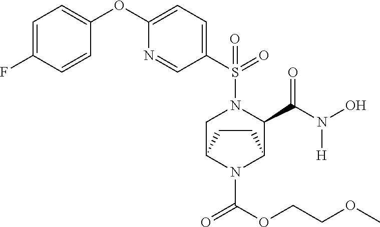 [SMILES: COCCOC(=O)N1[C@@H]2CC[C@H]1[C@H](C(=O)NO)N(S(=O)(=O)c1ccc(Oc3ccc(F)cc3)nc1)C2] | (1S,2R,5R)-2-methoxyethyl 3-((6-(4- fluorophenoxy)pyridin-3-yl)sulfonyl)-2- (hydroxycarbamoyl)-3,8- diazabicyclo[3.2.1]octane-8-carboxylate |
| 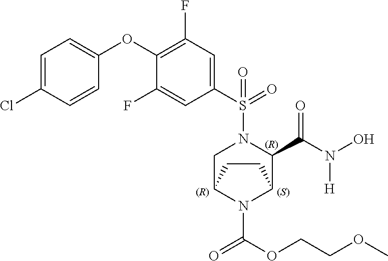 | (1S,2R,5R)-2-methoxyethyl 3-((4-(4- chlorophenoxy)-3,5-difluorophenyl)sulfonyl)- 2-(hydroxycarbamoyl)-3,8- diazabicyclo[3.2.1]octane-8-carboxylate |
| 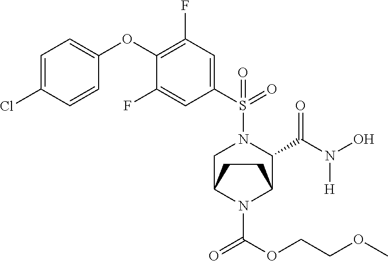 [SMILES: COCCOC(=O)N1[C@H]2CC[C@@H]1[C@@H](C(=O)NO)N(S(=O)(=O)c1cc(F)c(Oc3ccc(Cl)cc3)c(F)c1)C2] | 2-methoxyethyl (1R,2S,5S)-3-((4-(4- chlorophenoxy)-3,5-difluorophenyl)sulfonyl)- 2-(hydroxycarbamoyl)-3,8- diazabicyclo[3.2.1]octane-8-carboxylate |
| 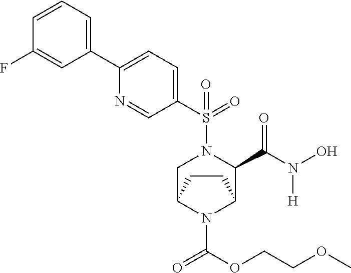 [SMILES: COCCOC(=O)N1[C@@H]2CC[C@H]1[C@H](C(=O)NO)N(S(=O)(=O)c1ccc(-c3cccc(F)c3)nc1)C2] | 2-methoxyethyl (1S,2R,5R)-3-((6-(3- fluorophenyl)pyridin-3-yl)sulfonyl)-2- (hydroxycarbamoyl)-3,8- diazabicyclo[3.2. 1]octane-8-carboxylate |
| 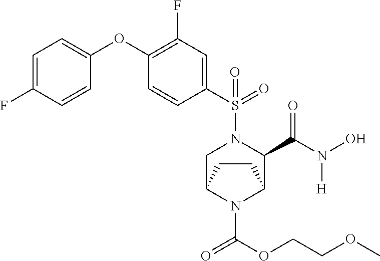 [SMILES: COCCOC(=O)N1[C@@H]2CC[C@H]1[C@H](C(=O)NO)N(S(=O)(=O)c1ccc(Oc3ccc(F)cc3)c(F)c1)C2] | 2-methoxyethyl (1S,2R,5R)-3-((3-fluoro-4-(4- fluorophenoxy)phenyl)sulfonyl)-2- (hydroxycarbamoyl)-3,8- diazabicyclo[3.2.1]octane-8-carboxylate |
| 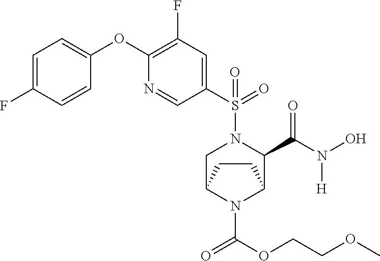 [SMILES: COCCOC(=O)N1[C@@H]2CC[C@H]1[C@H](C(=O)NO)N(S(=O)(=O)c1cnc(Oc3ccc(F)cc3)c(F)c1)C2] | 2-methoxyethyl (1S,2R,5R)-3-((5-fluoro-6-(4- fluorophenoxy)pyridin-3-yl)sulfonyl)-2- (hydroxycarbamoyl)-3,8- diazabicyclo[3.2.1]octane-8-carboxylate |
| 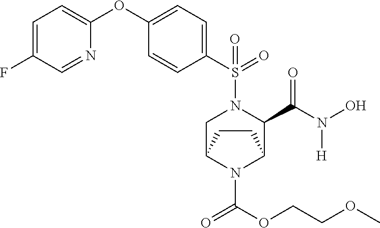 [SMILES: COCCOC(=O)N1[C@@H]2CC[C@H]1[C@H](C(=O)NO)N(S(=O)(=O)c1ccc(Oc3ccc(F)cn3)cc1)C2] | 2-methoxyethyl (1S,2R,5R)-3-((4-((5- fluoropyridin-2-yl)oxy)phenyl)sulfonyl)-2- (hydroxycarbamoyl)-3,8- diazabicyclo[3.2.1]octane-8-carboxylate |
| 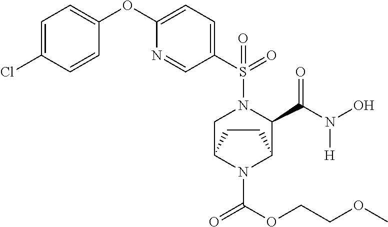 [SMILES: COCCOC(=O)N1[C@@H]2CC[C@H]1[C@H](C(=O)NO)N(S(=O)(=O)c1ccc(Oc3ccc(Cl)cc3)nc1)C2] | 2-methoxyethyl (1S,2R,5R)-3-((6-(4- chlorophenoxy)pyridin-3-yl)sulfonyl)-2- (hydroxycarbamoyl)-3,8- diazabicyclo[3.2.1]octane-8-carboxylate |
| 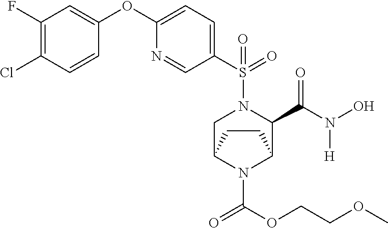 [SMILES: COCCOC(=O)N1[C@@H]2CC[C@H]1[C@H](C(=O)NO)N(S(=O)(=O)c1ccc(Oc3ccc(Cl)c(F)c3)nc1)C2] | 2-methoxyethyl (1S,2R,5R)-3-((6-(4-chloro-3- fluorophenoxy)pyridin-3-yl)sulfonyl)-2- (hydroxycarbamoyl)-3,8- diazabicyclo[3.2.1]octane-8-carboxylate |
| 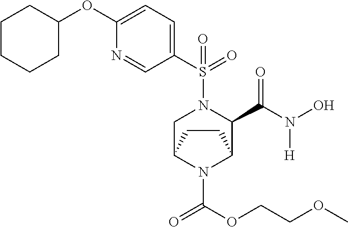 [SMILES: COCCOC(=O)N1[C@@H]2CC[C@H]1[C@H](C(=O)NO)N(S(=O)(=O)c1ccc(OC3CCCCC3)nc1)C2] | 2-methoxyethyl (1S,2R,5R)-3-((6- (cyclohexyloxy)pyridin-3-yl)sulfonyl)-2- (hydroxycarbamoyl)-3,8- diazabicyclo[3.2.1]octane-8-carboxylate |
| 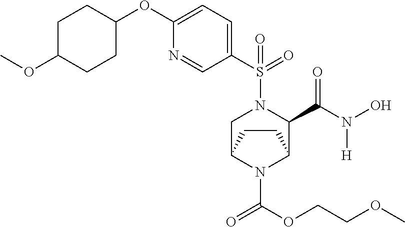 | 2-methoxyethyl (1S,2R,5R)-2- (hydroxycarbamoyl)-3-((6-((4- methoxycyclohexyl)oxy)pyridin-3- yl)sulfony1)-3,8-diazabicyclo[3.2.1]octane-8- carboxylate |
| 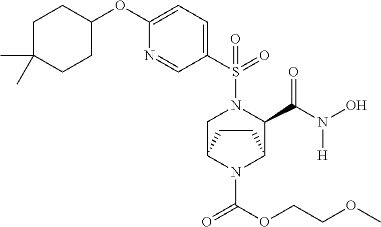 | 2-methoxyethyl (1S,2R,5R)-3-((6-((4,4- dimethylcyclohexyl)oxy)pyridin-3- yl)sulfonyl)-2-(hydroxycarbamoyl)-3,8- diazabicyclo[3.2.1]octane-8-carboxylate |
| 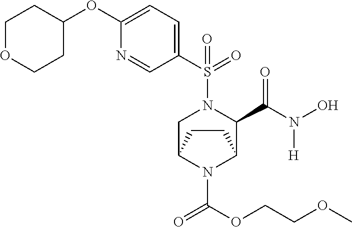 [SMILES: COCCOC(=O)N1[C@@H]2CC[C@H]1[C@H](C(=O)NO)N(S(=O)(=O)c1ccc(OC3CCOCC3)nc1)C2] | 2-methoxyethyl (1S,2R,5R)-2- (hydroxycarbamoyl)-3-((6-((tetrahydro-2H- pyran-4-yl)oxy)pyridin-3-yl)sulfonyl)-3,8- diazabicyclo[3.2.1]octane-8-carboxylate |
| 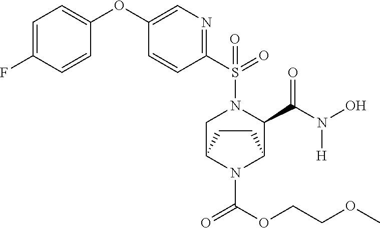 [SMILES: COCCOC(=O)N1[C@@H]2CC[C@H]1[C@H](C(=O)NO)N(S(=O)(=O)c1ccc(Oc3ccc(F)cc3)cn1)C2] | 2-methoxyethyl (1S,2R,5R)-3-((5-(4- fluorophenoxy)pyridin-2-yl)sulfonyl)-2- (hydroxycarbamoyl)-3,8- diazabicyclo[3.2.1]octane-8-carboxylate |
| 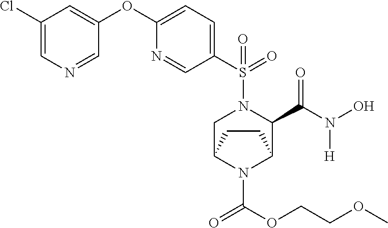 [SMILES: COCCOC(=O)N1[C@@H]2CC[C@H]1[C@H](C(=O)NO)N(S(=O)(=O)c1ccc(Oc3cncc(Cl)c3)nc1)C2] | (1S,2R,5R)-2-methoxyethyl 3-((6-((5- chloropyridin-3-yl)oxy)pyridin-3-yl)sulfonyl)- 2-(hydroxycarbamoyl)-3,8- diazabicyclo[3.2.1]octane-8-carboxylate |
| 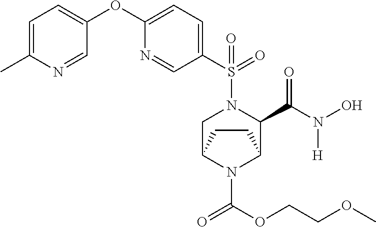 | 2-methoxyethyl (1S,2R,5R)-2- (hydroxycarbamoyl)-3-((6-((6-methylpyridin- 3-yl)oxy)pyridin-3-yl)sulfonyl)-3,8- diazabicyclo[3.2.1]octane-8-carboxylate |
| 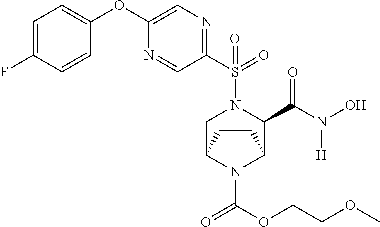 [SMILES: COCCOC(=O)N1[C@@H]2CC[C@H]1[C@H](C(=O)NO)N(S(=O)(=O)c1cnc(Oc3ccc(F)cc3)cn1)C2] | 2-methoxyethyl (1S,2R,5R)-3-((5-(4- fluorophenoxy)pyrazin-2-yl)sulfonyl)-2- (hydroxycarbamoyl)-3,8- diazabicyclo[3.2.1]octane-8-carboxylate |
| 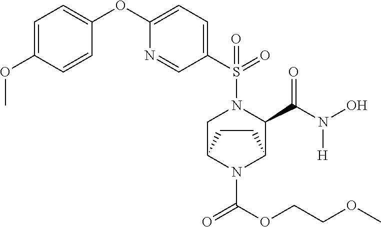 [SMILES: COCCOC(=O)N1[C@@H]2CC[C@H]1[C@H](C(=O)NO)N(S(=O)(=O)c1ccc(Oc3ccc(OC)cc3)nc1)C2] | 2-methoxyethyl (1S,2R,5R)-2- (hydroxycarbamoyl)-3-((6-(4- methoxyphenoxy)pyridin-3-yl)sulfonyl)-3,8- diazabicyclo[3.2.1]octane-8-carboxylate |
| 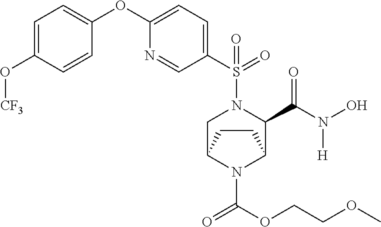 [SMILES: COCCOC(=O)N1[C@@H]2CC[C@H]1[C@H](C(=O)NO)N(S(=O)(=O)c1ccc(Oc3ccc(OC(F)(F)F)cc3)nc1)C2] | 2-methoxyethyl (1S,2R,5R)-2- (hydroxycarbamoyl)-3-((6-(4- (trifluoromethoxy)phenoxy)pyridin-3- yl)sulfonyl)-3,8-diazabicyclo[3.2.1]octane-8- carboxylate |
| 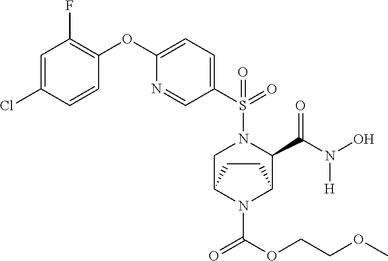 [SMILES: COCCOC(=O)N1[C@@H]2CC[C@H]1[C@H](C(=O)NO)N(S(=O)(=O)c1ccc(Oc3ccc(Cl)cc3F)nc1)C2] | 2-methoxyethyl (1S,2R,5R)-3-((6-(4-chloro-2- fluorophenoxy)pyridin-3-yl)sulfonyl)-2- (hydroxycarbamoyl)-3,8- diazabicyclo[3.2.1]octane-8-carboxylate |
| 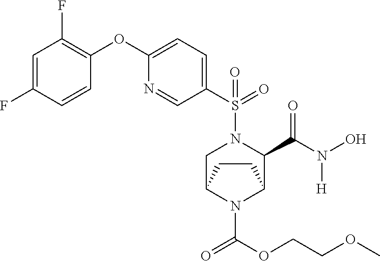 [SMILES: COCCOC(=O)N1[C@@H]2CC[C@H]1[C@H](C(=O)NO)N(S(=O)(=O)c1ccc(Oc3ccc(F)cc3F)nc1)C2] | 2-methoxyethyl (1S,2R,5R)-3-((6-(2,4- difluorophenoxy)pyridin-3-yl)sulfonyl)-2- (hydroxycarbamoyl)-3,8- diazabicyclo[3.2.1]octane-8-carboxylate |
| 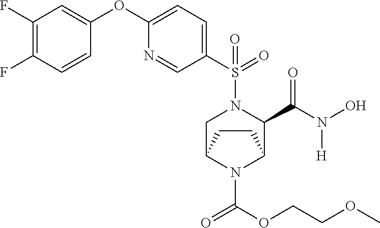 [SMILES: COCCOC(=O)N1[C@@H]2CC[C@H]1[C@H](C(=O)NO)N(S(=O)(=O)c1ccc(Oc3ccc(F)c(F)c3)nc1)C2] | 2-methoxyethyl (1S,2R,5R)-3-((6-(3,4- difluorophenoxy)pyridin-3-yl)sulfonyl)-2- (hydroxycarbamoyl)-3,8- diazabicyclo[3.2.1]octane-8-carboxylate |
| 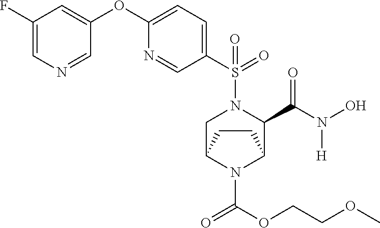 [SMILES: COCCOC(=O)N1[C@@H]2CC[C@H]1[C@H](C(=O)NO)N(S(=O)(=O)c1ccc(Oc3cncc(F)c3)nc1)C2] | 2-methoxyethyl (1S,2R,5R)-3-((6-((5- fluoropyridin-3-yl)oxy)pyridin-3-yl)sulfonyl)- 2-(hydroxycarbamoyl)-3,8- diazabicyclo[3.2.1]octane-8-carboxylate |
| 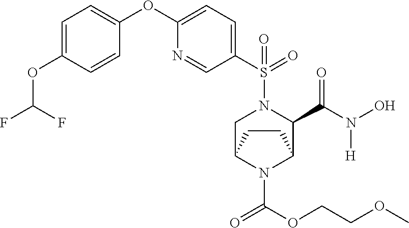 [SMILES: COCCOC(=O)N1[C@@H]2CC[C@H]1[C@H](C(=O)NO)N(S(=O)(=O)c1ccc(Oc3ccc(OC(F)F)cc3)nc1)C2] | 2-methoxyethyl (1S,2R,5R)-3-((6-(4- (difluoromethoxy)phenoxy)pyridin-3- yl)sulfonyl)-2-(hydroxycarbamoyl)-3,8- diazabicyclo[3.2.1]octane-8-carboxylate |
| 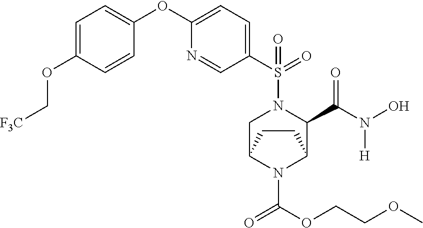 [SMILES: COCCOC(=O)N1[C@@H]2CC[C@H]1[C@H](C(=O)NO)N(S(=O)(=O)c1ccc(Oc3ccc(OCC(F)(F)F)cc3)nc1)C2] | 2-methoxyethyl (1S,2R,5R)-2- (hydroxycarbamoyl)-3-((6-(4-(2,2,2- trifluoroethoxy)phenoxy)pyridin-3- yl)sulfonyl)-3,8-diazabicyclo[3.2.1]octane-8- carboxylate |
| 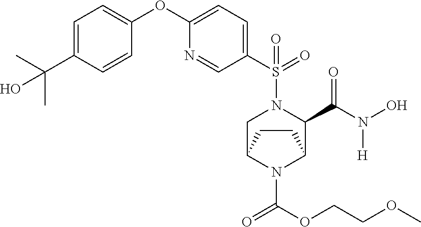 [SMILES: COCCOC(=O)N1[C@@H]2CC[C@H]1[C@H](C(=O)NO)N(S(=O)(=O)c1ccc(Oc3ccc(C(C)(C)O)cc3)nc1)C2] | 2-methoxyethyl (1S,2R,5R)-2- (hydroxycarbamoyl)-3-((6-(4-(2- hydroxypropan-2-yl)phenoxy)pyridin-3- yl)sulfonyl)-3,8-diazabicyclo[3.2.1]octane-8- carboxylate |
| 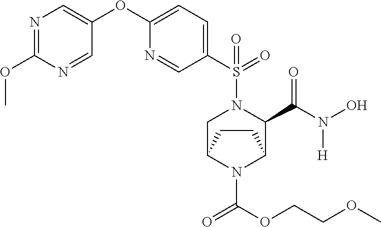 [SMILES: COCCOC(=O)N1[C@@H]2CC[C@H]1[C@H](C(=O)NO)N(S(=O)(=O)c1ccc(Oc3cnc(OC)nc3)nc1)C2] | 2-methoxyethyl (1S,2R,5R)-2- (hydroxycarbamoy1)-3-((6-((2- methoxypyrimidin-5-yl)oxy)pyridin-3- yl)sulfony1)-3,8-diazabicyclo[3.2.1]octane-8- carboxylate |
| 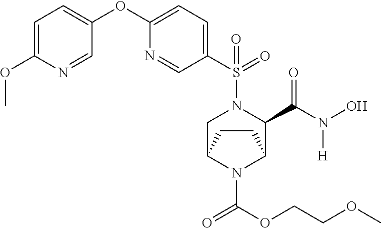 [SMILES: COCCOC(=O)N1[C@@H]2CC[C@H]1[C@H](C(=O)NO)N(S(=O)(=O)c1ccc(Oc3ccc(OC)nc3)nc1)C2] | 2-methoxyethyl (1S,2R,5R)-2- (hydroxycarbamoyl)-3-((6-((6- methoxypyridin-3-yl)oxy)pyridin-3- yl)sulfonyl)-3,8-diazabicyclo[3.2.1]octane-8- carboxylate |
| 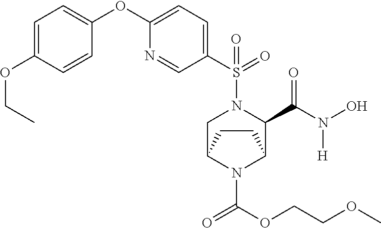 [SMILES: CCOc1ccc(Oc2ccc(S(=O)(=O)N3C[C@H]4CC[C@@H]([C@@H]3C(=O)NO)N4C(=O)OCCOC)cn2)cc1] | 2-methoxyethyl (1S,2R,5R)-3-((6-(4- ethoxyphenoxy)pyridin-3-yl)sulfonyl)-2- (hydroxycarbamoyl)-3,8- diazabicyclo[3.2.1]octane-8-carboxylate |
| 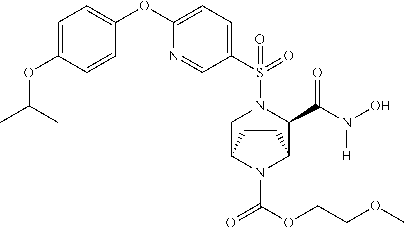 [SMILES: COCCOC(=O)N1[C@@H]2CC[C@H]1[C@H](C(=O)NO)N(S(=O)(=O)c1ccc(Oc3ccc(OC(C)C)cc3)nc1)C2] | 2-methoxyethyl (1S,2R,5R)-2- (hydroxycarbamoy1)-3-((6-(4- isopropoxyphenoxy)pyridin-3-yl)sulfony1)-3,8- diazabicyclo[3.2.1]octane-8-carboxylate |
| 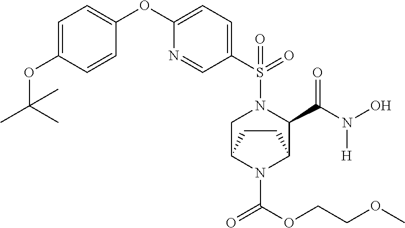 [SMILES: COCCOC(=O)N1[C@@H]2CC[C@H]1[C@H](C(=O)NO)N(S(=O)(=O)c1ccc(Oc3ccc(OC(C)(C)C)cc3)nc1)C2] | 2-methoxyethyl (1S,2R,5R)-3-((6-(4-(tert- butoxy)phenoxy)pyridin-3-yl)sulfonyl)-2- (hydroxycarbamoyl)-3,8- diazabicyclo[3.2.1]octane-8-carboxylate |
| 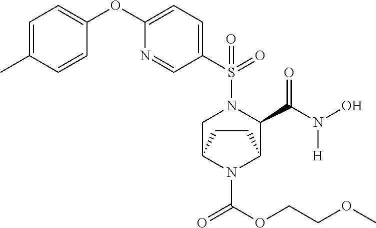 | 2-methoxyethyl (1S,2R,5R)-2- (hydroxycarbamoyl)-3-((6-(p-tolyloxy)pyridin- 3-yl)sulfonyl)-3,8-diazabicyclo[3.2.1]octane-8- carboxylate |
| 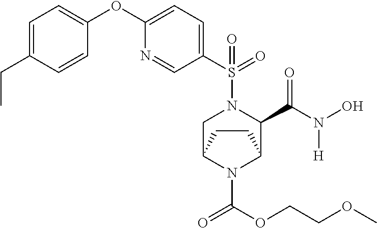 [SMILES: CCc1ccc(Oc2ccc(S(=O)(=O)N3C[C@H]4CC[C@@H]([C@@H]3C(=O)NO)N4C(=O)OCCOC)cn2)cc1] | 2-methoxyethyl (1S,2R,5R)-3-((6-(4- ethylphenoxy)pyridin-3-yl)sulfonyl)-2- (hydroxycarbamoyl)-3,8- diazabicyclo[3.2.1]octane-8-carboxylate |
| 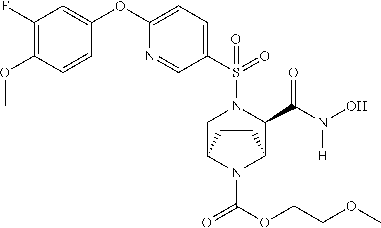 [SMILES: COCCOC(=O)N1[C@@H]2CC[C@H]1[C@H](C(=O)NO)N(S(=O)(=O)c1ccc(Oc3ccc(OC)c(F)c3)nc1)C2] | 2-methoxyethyl (1S,2R,5R)-3-((6-(3-fluoro-4- methoxyphenoxy)pyridin-3-yl)sulfonyl)-2- (hydroxycarbamoyl)-3,8- diazabicyclo[3.2.1]octane-8-carboxylate |
| 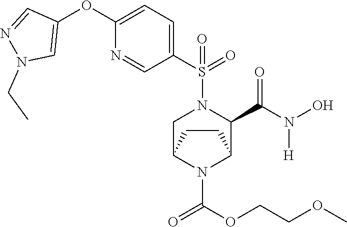 [SMILES: CCn1cc(Oc2ccc(S(=O)(=O)N3C[C@H]4CC[C@@H]([C@@H]3C(=O)NO)N4C(=O)OCCOC)cn2)cn1] | 2-methoxyethyl (1S,2R,5R)-3-((6-((1-ethyl- 1H-pyrazol-4-yl)oxy)pyridin-3-yl)sulfonyl)-2- (hydroxycarbamoyl)-3,8- diazabicyclo[3.2.1]octane-8-carboxylate |
| 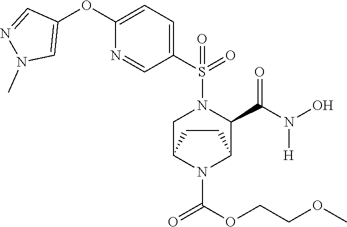 [SMILES: COCCOC(=O)N1[C@@H]2CC[C@H]1[C@H](C(=O)NO)N(S(=O)(=O)c1ccc(Oc3cnn(C)c3)nc1)C2] | 2-methoxyethyl (1S,2R,5R)-2- (hydroxycarbamoyl)-3-((6-((1-methyl-1H- pyrazol-4-yl)oxy)pyridin-3-yl)sulfonyl)-3,8- diazabicyclo[3.2.1]octane-8-carboxylate |
| 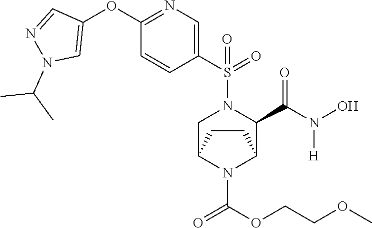 [SMILES: COCCOC(=O)N1[C@@H]2CC[C@H]1[C@H](C(=O)NO)N(S(=O)(=O)c1ccc(Oc3cnn(C(C)C)c3)nc1)C2] | 2-methoxyethyl (1S,2R,5R)-2- (hydroxycarbamoyl)-3-((6-((1-isopropyl-1H- pyrazol-4-yl)oxy)pyridin-3-yl)sulfonyl)-3,8- diazabicyclo[3.2.1]octane-8-carboxylate |
| 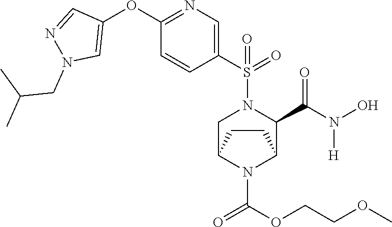 [SMILES: COCCOC(=O)N1[C@@H]2CC[C@H]1[C@H](C(=O)NO)N(S(=O)(=O)c1ccc(Oc3cnn(CC(C)C)c3)nc1)C2] | 2-methoxyethyl (1S,2R,5R)-2- (hydroxycarbamoyl)-3-((6-((1-isobutyl-1H- pyrazol-4-yl)oxy)pyridin-3-yl)sulfonyl)-3,8- diazabicyclo[3.2.1]octane-8-carboxylate |
| 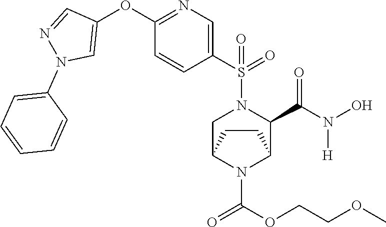 [SMILES: COCCOC(=O)N1[C@@H]2CC[C@H]1[C@H](C(=O)NO)N(S(=O)(=O)c1ccc(Oc3cnn(-c4ccccc4)c3)nc1)C2] | 2-methoxyethyl (1S,2R,5R)-2- (hydroxycarbamoyl)-3-((6-((1-phenyl-1H- pyrazol-4-yl)oxy)pyridin-3-yl)sulfonyl)-3,8- diazabicyclo[3.2.1]octane-8-carboxylate |
| 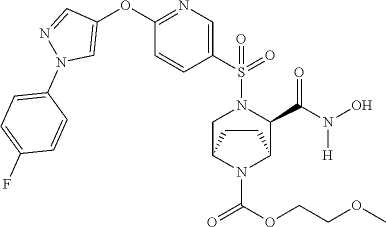 [SMILES: COCCOC(=O)N1[C@@H]2CC[C@H]1[C@H](C(=O)NO)N(S(=O)(=O)c1ccc(Oc3cnn(-c4ccc(F)cc4)c3)nc1)C2] | 2-methoxyethyl (1S,2R,5R)-3-((6-((1-(4- fluorophenyl)-1H-pyrazol-4-yl)oxy)pyridin-3- yl)sulfonyl)-2-(hydroxycarbamoyl)-3,8- diazabicyclo[3.2.1]octane-8-carboxylate |
| 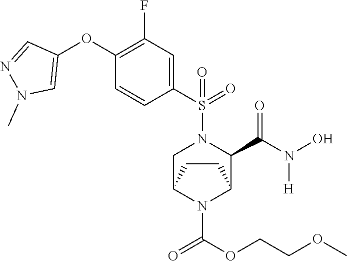 [SMILES: COCCOC(=O)N1[C@@H]2CC[C@H]1[C@H](C(=O)NO)N(S(=O)(=O)c1ccc(Oc3cnn(C)c3)c(F)c1)C2] | 2-methoxyethyl (1S,2R,5R)-3-((3-fluoro-4-((1- methyl-1H-pyrazol-4-yl)oxy)phenyl)sulfonyl)- 2-(hydroxycarbamoyl)-3,8- diazabicyclo[3.2.1]octane-8-carboxylate |
| 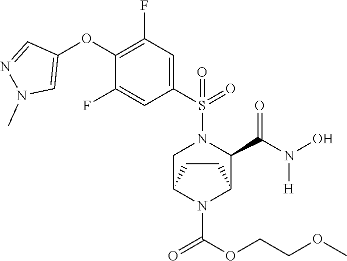 [SMILES: COCCOC(=O)N1[C@@H]2CC[C@H]1[C@H](C(=O)NO)N(S(=O)(=O)c1cc(F)c(Oc3cnn(C)c3)c(F)c1)C2] | 2-methoxyethyl (1S,2R,5R)-3-((3,5-difluoro-4- ((1-methyl-1H-pyrazol-4- yl)oxy)phenyl)sulfonyl)-2- (hydroxycarbamoyl)-3,8- diazabicyclo[3.2.1]octane-8-carboxylate |
| 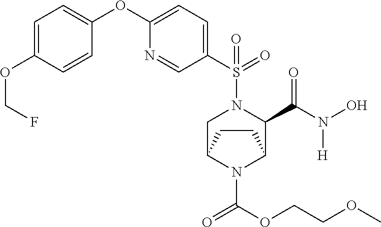 [SMILES: COCCOC(=O)N1[C@@H]2CC[C@H]1[C@H](C(=O)NO)N(S(=O)(=O)c1ccc(Oc3ccc(OCF)cc3)nc1)C2] | 2-methoxyethyl (1S,2R,5R)-3-((6-(4- (fluoromethoxy)phenoxy)pyridin-3- yl)sulfonyl)-2-(hydroxycarbamoyl)-3,8- diazabicyclo[3.2.1]octane-8-carboxylate |
| 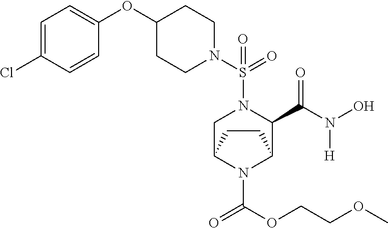 [SMILES: COCCOC(=O)N1[C@@H]2CC[C@H]1[C@H](C(=O)NO)N(S(=O)(=O)N1CCC(Oc3ccc(Cl)cc3)CC1)C2] | (1S,2R,5R)-2-methoxyethyl 3-((4-(4- chlorophenoxy)piperidin-1-yl)sulfonyl)-2- (hydroxycarbamoyl)-3,8- diazabicyclo[3.2.1]octane-8-carboxylate |
| 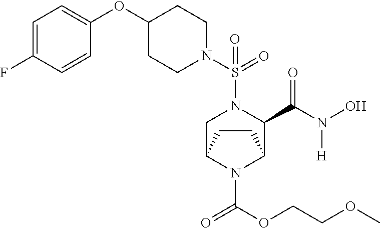 [SMILES: COCCOC(=O)N1[C@@H]2CC[C@H]1[C@H](C(=O)NO)N(S(=O)(=O)N1CCC(Oc3ccc(F)cc3)CC1)C2] | 2-methoxyethyl (1S,2R,5R)-3-((4-(4- fluorophenoxy)piperidin-1-yl)sulfonyl)-2- (hydroxycarbamoyl)-3,8- diazabicyclo[3.2.1]octane-8-carboxylate |
| 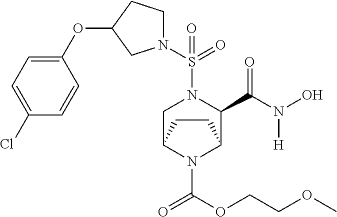 [SMILES: COCCOC(=O)N1[C@@H]2CC[C@H]1[C@H](C(=O)NO)N(S(=O)(=O)N1CCC(Oc3ccc(Cl)cc3)C1)C2] | 2-methoxyethyl (1S,2R,5R)-3-((3-(4- chlorophenoxy)pyrrolidin-1-yl)sulfonyl)-2- (hydroxycarbamoyl)-3,8- diazabicyclo[3.2.1]octane-8-carboxylate |
| 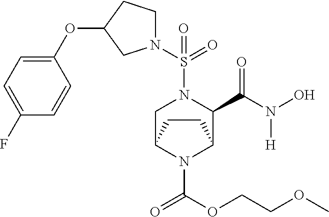 [SMILES: COCCOC(=O)N1[C@@H]2CC[C@H]1[C@H](C(=O)NO)N(S(=O)(=O)N1CCC(Oc3ccc(F)cc3)C1)C2] | 2-methoxyethyl (1S,2R,5R)-3-((3-(4- fluorophenoxy)pyrrolidin-1-yl)sulfonyl)-2- (hydroxycarbamoyl)-3,8- diazabicyclo[3.2.1]octane-8-carboxylate |
| 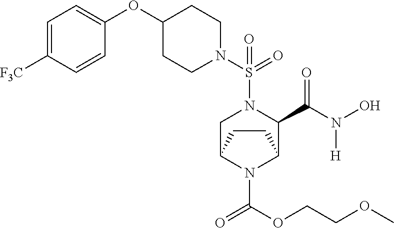 [SMILES: COCCOC(=O)N1[C@@H]2CC[C@H]1[C@H](C(=O)NO)N(S(=O)(=O)N1CCC(Oc3ccc(C(F)(F)F)cc3)CC1)C2] | 2-methoxyethyl (1S,2R,5R)-2- (hydroxycarbamoyl)-3-((4-(4- (trifluoromethyl)phenoxy)piperidin-1- yl)sulfonyl)-3,8-diazabicyclo[3.2.1]octane-8- carboxylate |
| 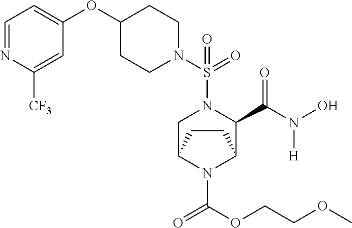 [SMILES: COCCOC(=O)N1[C@@H]2CC[C@H]1[C@H](C(=O)NO)N(S(=O)(=O)N1CCC(Oc3ccnc(C(F)(F)F)c3)CC1)C2] | 2-methoxyethyl (1S,2R,5R)-2- (hydroxycarbamoyl)-3-((4-((2- (trifluoromethyl)pyridin-4-yl)oxy)piperidin-1- yl)sulfonyl)-3,8-diazabicyclo[3.2.1]octane-8- carboxylate |
| 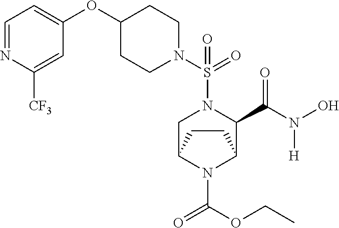 [SMILES: CCOC(=O)N1[C@@H]2CC[C@H]1[C@H](C(=O)NO)N(S(=O)(=O)N1CCC(Oc3ccnc(C(F)(F)F)c3)CC1)C2] | ethyl (1S,2R,5R)-2-(hydroxycarbamoyl)-3-((4- ((2-(trifluoromethyl)pyridin-4- yl)oxy)piperidin-1-yl)sulfonyl)-3,8- diazabicyclo[3.2.1]octane-8-carboxylate |
| 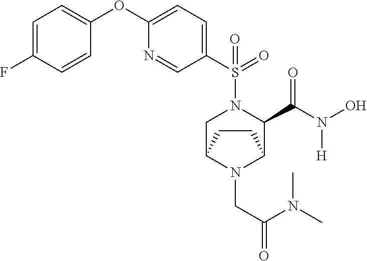 [SMILES: CN(C)C(=O)CN1[C@@H]2CC[C@H]1[C@H](C(=O)NO)N(S(=O)(=O)c1ccc(Oc3ccc(F)cc3)nc1)C2] | (1S,2R,5R)-8-(2-(dimethylamino)-2-oxoethyl)- 3-((6-(4-fluorophenoxy)pyridin-3-yl)sulfonyl)- N-hydroxy-3,8-diazabicyclo[3.2.1]octane-2- carboxamide |
| 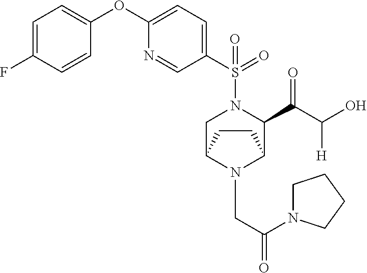 [SMILES: O=C(CO)[C@H]1[C@@H]2CC[C@H](CN1S(=O)(=O)c1ccc(Oc3ccc(F)cc3)nc1)N2CC(=O)N1CCCC1] | (1S,2R,5R)-3-((6-(4-fluorophenoxy)pyridin-3- yl)sulfonyl)-N-hydroxy-8-(2-oxo-2- (pyrrolidin-1-yl)ethyl)-3,8- diazabicyclo[3.2.1]octane-2-carboxamide |
| 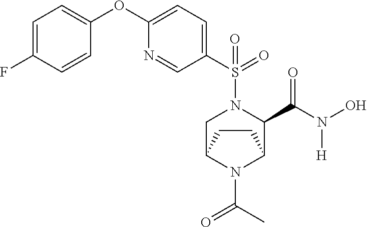 [SMILES: CC(=O)N1[C@@H]2CC[C@H]1[C@H](C(=O)NO)N(S(=O)(=O)c1ccc(Oc3ccc(F)cc3)nc1)C2] | (1S,2R,5R)-8-acetyl-3-((6-(4- fluorophenoxy)pyridin-3-yl)sulfonyl)-N- hydroxy-3,8-diazabicyclo[3.2.1]octane-2- carboxamide |
| 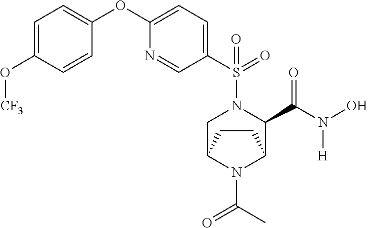 [SMILES: CC(=O)N1[C@@H]2CC[C@H]1[C@H](C(=O)NO)N(S(=O)(=O)c1ccc(Oc3ccc(OC(F)(F)F)cc3)nc1)C2] | (1S,2R,5R)-8-acetyl-N-hydroxy-3-((6-(4- (trifluoromethoxy)phenoxy)pyridin-3- yl)sulfonyl)-3,8-diazabicyclo[3.2. 1]octane-2- carboxamide |
| 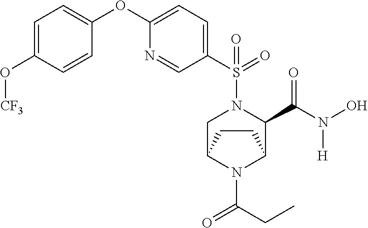 [SMILES: CCC(=O)N1[C@@H]2CC[C@H]1[C@H](C(=O)NO)N(S(=O)(=O)c1ccc(Oc3ccc(OC(F)(F)F)cc3)nc1)C2] | (1S,2R,5R)-N-hydroxy-8-propionyl-3-((6-(4- (trifluoromethoxy)phenoxy)pyridin-3- yl)sulfonyl)-3,8-diazabicyclo[3.2.1]octane-2- carboxamide |
| 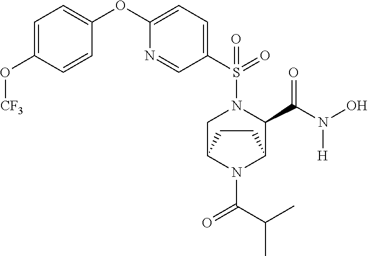 [SMILES: CC(C)C(=O)N1[C@@H]2CC[C@H]1[C@H](C(=O)NO)N(S(=O)(=O)c1ccc(Oc3ccc(OC(F)(F)F)cc3)nc1)C2] | (1S,2R,5R)-N-hydroxy-8-isobutyryl-3-((6-(4- (trifluoromethoxy)phenoxy)pyridin-3- yl)sulfonyl)-3,8-diazabicyclo[3.2.1]octane-2- carboxamide |
| 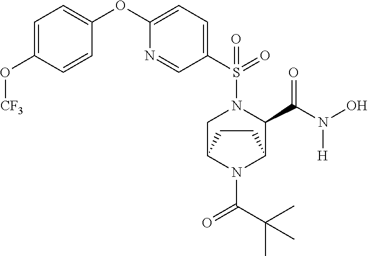 [SMILES: CC(C)(C)C(=O)N1[C@@H]2CC[C@H]1[C@H](C(=O)NO)N(S(=O)(=O)c1ccc(Oc3ccc(OC(F)(F)F)cc3)nc1)C2] | (1S,2R,5R)-N-hydroxy-8-pivaloyl-3-((6-(4- (trifluoromethoxy)phenoxy)pyridin-3- yl)sulfonyl)-3,8-diazabicyclo[3.2.1]octane-2- carboxamide |
| 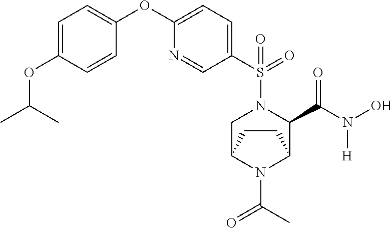 [SMILES: CC(=O)N1[C@@H]2CC[C@H]1[C@H](C(=O)NO)N(S(=O)(=O)c1ccc(Oc3ccc(OC(C)C)cc3)nc1)C2] | (1S,2R,5R)-8-acetyl-N-hydroxy-3-((6-(4- isopropoxyphenoxy)pyridin-3-yl)sulfonyl)-3,8- diazabicyclo[3.2.1]octane-2-carboxamide |
| 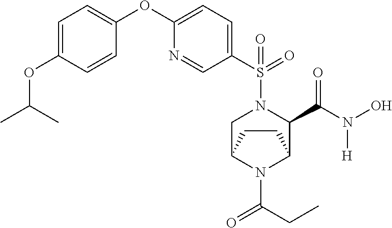 [SMILES: CCC(=O)N1[C@@H]2CC[C@H]1[C@H](C(=O)NO)N(S(=O)(=O)c1ccc(Oc3ccc(OC(C)C)cc3)nc1)C2] | (1S,2R,5R)-N-hydroxy-3-((6-(4- isopropoxyphenoxy)pyridin-3-yl)sulfonyl)-8- propionyl-3,8-diazabicyclo[3.2.1]octane-2- carboxamide |
| 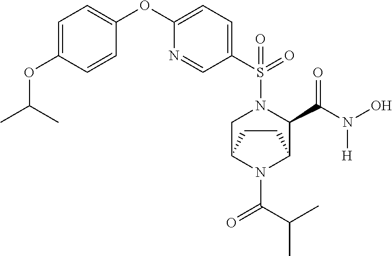 [SMILES: CC(C)Oc1ccc(Oc2ccc(S(=O)(=O)N3C[C@H]4CC[C@@H]([C@@H]3C(=O)NO)N4C(=O)C(C)C)cn2)cc1] | (1S,2R,5R)-N-hydroxy-8-isobutyryl-3-((6-(4- isopropoxyphenoxy)pyridin-3-yl)sulfonyl)-3,8- diazabicyclo[3.2.1]octane-2-carboxamide |
| 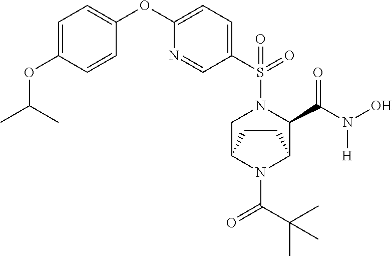 [SMILES: CC(C)Oc1ccc(Oc2ccc(S(=O)(=O)N3C[C@H]4CC[C@@H]([C@@H]3C(=O)NO)N4C(=O)C(C)(C)C)cn2)cc1] | (1S,2R,5R)-N-hydroxy-3-((6-(4- isopropoxyphenoxy)pyridin-3-yl)sulfonyl)-8- pivaloyl-3,8-diazabicyclo[3.2.1]octane-2- carboxamide |
| 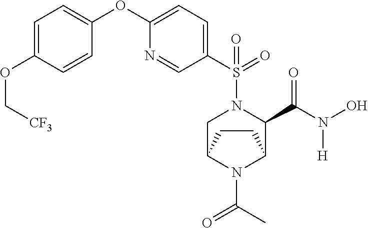 [SMILES: CC(=O)N1[C@@H]2CC[C@H]1[C@H](C(=O)NO)N(S(=O)(=O)c1ccc(Oc3ccc(OCC(F)(F)F)cc3)nc1)C2] | (1S,2R,5R)-8-acetyl-N-hydroxy-3-((6-(4- (2,2,2-trifluoroethoxy)phenoxy)pyridin-3- yl)sulfonyl)-3,8-diazabicyclo[3.2.1]octane-2- carboxamide |
| 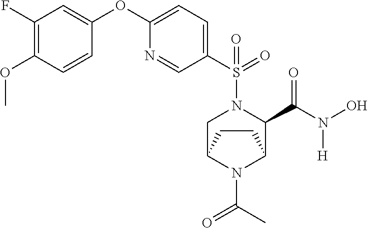 [SMILES: COc1ccc(Oc2ccc(S(=O)(=O)N3C[C@H]4CC[C@@H]([C@@H]3C(=O)NO)N4C(C)=O)cn2)cc1F] | (1S,2R,5R)-8-acetyl-3-((6-(3-fluoro-4- methoxyphenoxy)pyridin-3-yl)sulfonyl)-N- hydroxy-3,8-diazabicyclo[3.2.1]octane-2- carboxamide |
| 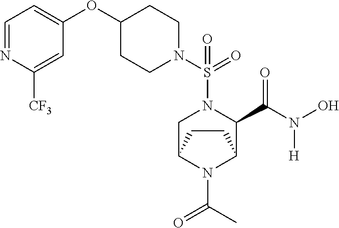 [SMILES: CC(=O)N1[C@@H]2CC[C@H]1[C@H](C(=O)NO)N(S(=O)(=O)N1CCC(Oc3ccnc(C(F)(F)F)c3)CC1)C2] | (1S,2R,5R)-8-acetyl-N-hydroxy-3-((4-((2- (trifluoromethyl)pyridin-4-yl)oxy)piperidin-1- yl)sulfonyl)-3,8-diazabicyclo[3.2.1]octane-2- carboxamide |
| 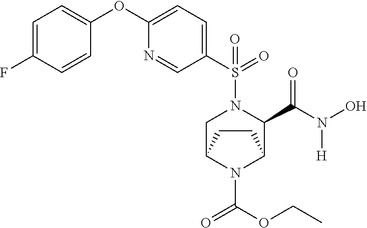 [SMILES: CCOC(=O)N1[C@@H]2CC[C@H]1[C@H](C(=O)NO)N(S(=O)(=O)c1ccc(Oc3ccc(F)cc3)nc1)C2] | ethyl (1S,2R,5R)-3-((6-(4- fluorophenoxy)pyridin-3-yl)sulfonyl)-2- (hydroxycarbamoyl)-3,8- diazabicyclo[3.2.1]octane-8-carboxylate |
| 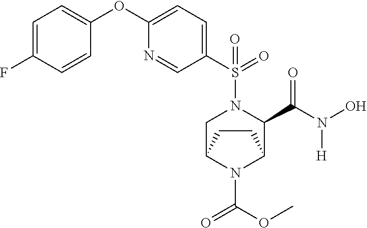 [SMILES: COC(=O)N1[C@@H]2CC[C@H]1[C@H](C(=O)NO)N(S(=O)(=O)c1ccc(Oc3ccc(F)cc3)nc1)C2] | methyl (1S,2R,5R)-3-((6-(4- fluorophenoxy)pyridin-3-yl)sulfonyl)-2- (hydroxycarbamoyl)-3,8- diazabicyclo[3.2.1]octane-8-carboxylate |
| 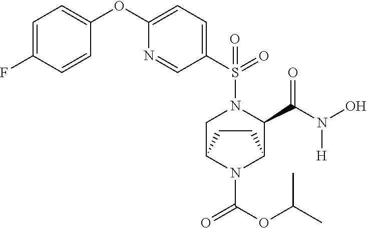 [SMILES: CC(C)OC(=O)N1[C@@H]2CC[C@H]1[C@H](C(=O)NO)N(S(=O)(=O)c1ccc(Oc3ccc(F)cc3)nc1)C2] | isopropyl (1S,2R,5R)-3-((6-(4- fluorophenoxy)pyridin-3-yl)sulfonyl)-2- (hydroxycarbamoyl)-3,8- diazabicyclo[3.2.1]octane-8-carboxylate |
| 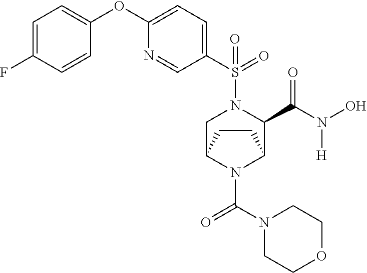 [SMILES: O=C(NO)[C@H]1[C@@H]2CC[C@H](CN1S(=O)(=O)c1ccc(Oc3ccc(F)cc3)nc1)N2C(=O)N1CCOCC1] | (1S,2R,5R)-3-((6-(4-fluorophenoxy)pyridin-3- yl)sulfony1)-N-hydroxy-8-(morpholine-4- carbonyl)-3,8-diazabicyclo[3.2.1]octane-2- carboxamide |
| 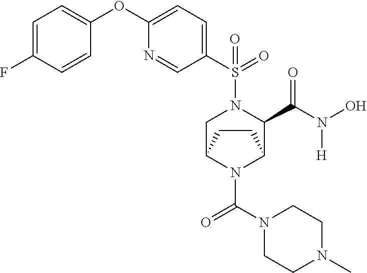 [SMILES: CN1CCN(C(=O)N2[C@@H]3CC[C@H]2[C@H](C(=O)NO)N(S(=O)(=O)c2ccc(Oc4ccc(F)cc4)nc2)C3)CC1] | (1S,2R,5R)-3-((6-(4-fluorophenoxy)pyridin-3- yl)sulfonyl)-N-hydroxy-8-(4- methylpiperazine-1-carbonyl)-3,8- diazabicyclo[3.2.1]octane-2-carboxamide |
| 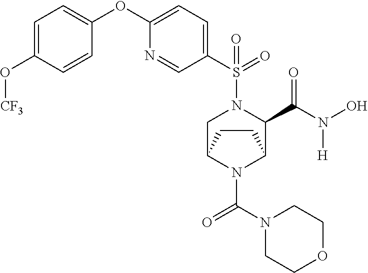 [SMILES: O=C(NO)[C@H]1[C@@H]2CC[C@H](CN1S(=O)(=O)c1ccc(Oc3ccc(OC(F)(F)F)cc3)nc1)N2C(=O)N1CCOCC1] | (1S,2R,5R)-N-hydroxy-8-(morpholine-4- carbonyl)-3-((6-(4- (trifluoromethoxy)phenoxy)pyridin-3- yl)sulfonyl)-3,8-diazabicyclo[3.2.1]octane-2- carboxamide |
| 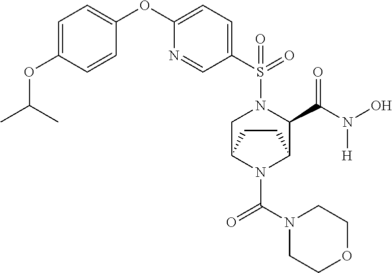 [SMILES: CC(C)Oc1ccc(Oc2ccc(S(=O)(=O)N3C[C@H]4CC[C@@H]([C@@H]3C(=O)NO)N4C(=O)N3CCOCC3)cn2)cc1] | (1S,2R,5R)-N-hydroxy-3-((6-(4- isopropoxyphenoxy)pyridin-3-yl)sulfonyl)-8- (morpholine-4-carbonyl)-3,8- diazabicyclo[3.2.1]octane-2-carboxamide |
| 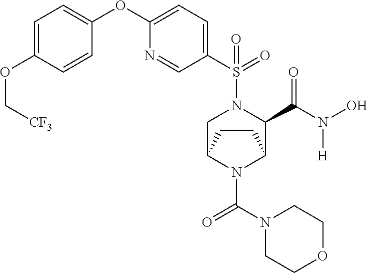 [SMILES: O=C(NO)[C@H]1[C@@H]2CC[C@H](CN1S(=O)(=O)c1ccc(Oc3ccc(OCC(F)(F)F)cc3)nc1)N2C(=O)N1CCOCC1] | (1S,2R,5R)-N-hydroxy-8-(morpholine-4- carbonyl)-3-((6-(4-(2,2,2- trifluoroethoxy)phenoxy)pyridin-3- yl)sulfonyl)-3,8-diazabicyclo[3.2.1]octane-2- carboxamide |
| 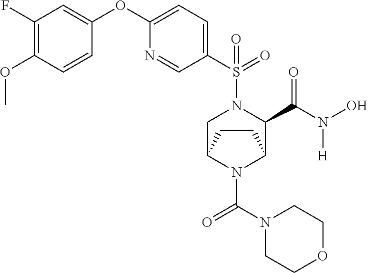 [SMILES: COc1ccc(Oc2ccc(S(=O)(=O)N3C[C@H]4CC[C@@H]([C@@H]3C(=O)NO)N4C(=O)N3CCOCC3)cn2)cc1F] | (1S,2R,5R)-3-((6-(3-fluoro-4- methoxyphenoxy)pyridin-3-yl)sulfonyl)-N- hydroxy-8-(morpholine-4-carbonyl)-3,8- diazabicyclo[3.2.1]octane-2-carboxamide |
| 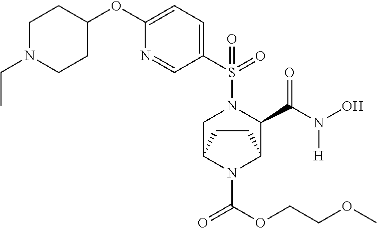 [SMILES: CCN1CCC(Oc2ccc(S(=O)(=O)N3C[C@H]4CC[C@@H]([C@@H]3C(=O)NO)N4C(=O)OCCOC)cn2)CC1] | 2-methoxyethyl (1S,2R,5R)-3-((6-((1- ethylpiperidin-4-yl)oxy)pyridin-3-yl)sulfonyl)- 2-(hydroxycarbamoyl)-3,8- diazabicyclo[3.2.1]octane-8-carboxylate |
| 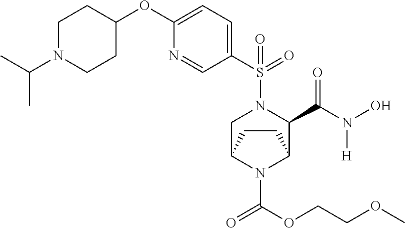 [SMILES: COCCOC(=O)N1[C@@H]2CC[C@H]1[C@H](C(=O)NO)N(S(=O)(=O)c1ccc(OC3CCN(C(C)C)CC3)nc1)C2] | 2-methoxyethyl (1S,2R,5R)-2- (hydroxycarbamoyl)-3-((6-((1- isopropylpiperidin-4-yl)oxy)pyridin-3- yl)sulfony1)-3,8-diazabicyclo[3.2.1]octane-8- carboxylate |
| 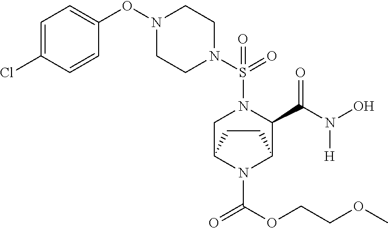 [SMILES: COCCOC(=O)N1[C@@H]2CC[C@H]1[C@H](C(=O)NO)N(S(=O)(=O)N1CCN(Oc3ccc(Cl)cc3)CC1)C2] | 2-methoxyethyl (1S,2R,5R)-3-((4-(4- chlorobenzyl)piperazin-1-yl)sulfonyl)-2- (hydroxycarbamoyl)-3,8- diazabicyclo[3.2.1]octane-8-carboxylate |
| 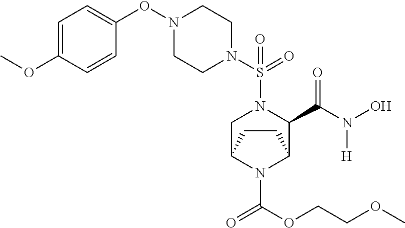 | 2-methoxyethyl (1S,2R,5R)-2- (hydroxycarbamoyl)-3-((4-(4- methoxybenzyl)piperazin-1-yl)sulfonyl)-3,8- diazabicyclo[3.2.1]octane-8-carboxylate |
| 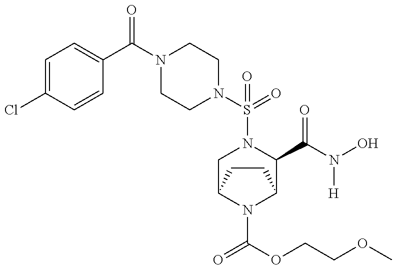 [SMILES: COCCOC(=O)N1[C@@H]2CC[C@H]1[C@H](C(=O)NO)N(S(=O)(=O)N1CCN(C(=O)c3ccc(Cl)cc3)CC1)C2] | 2-methoxyethyl (1S,2R,5R)-3-((4-(4- chlorobenzoyl)piperazin-1-yl)sulfonyl)-2- (hydroxycarbamoyl)-3,8- diazabicyclo[3.2.1]octane-8-carboxylate |
| 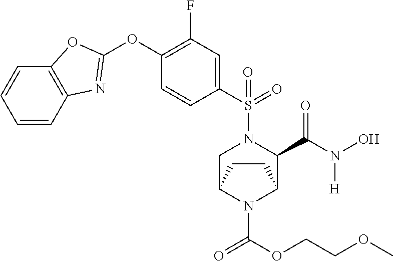 [SMILES: COCCOC(=O)N1[C@@H]2CC[C@H]1[C@H](C(=O)NO)N(S(=O)(=O)c1ccc(Oc3nc4ccccc4o3)c(F)c1)C2] | 2-methoxyethyl (1S,2R,5R)-3-((4- (benzo[d]oxazol-2-yloxy)-3- fluorophenyl)sulfony1)-2-(hydroxycarbamoyl)- 3,8-diazabicyclo[3.2.1]octane-8-carboxylate |
| 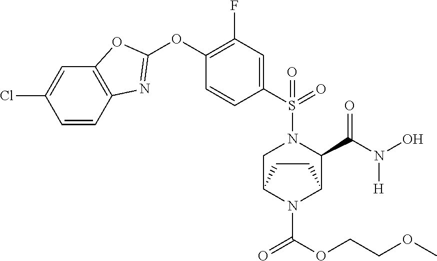 [SMILES: COCCOC(=O)N1[C@@H]2CC[C@H]1[C@H](C(=O)NO)N(S(=O)(=O)c1ccc(Oc3nc4ccc(Cl)cc4o3)c(F)c1)C2] | 2-methoxyethyl (1S,2R,5R)-3-((4-((6- chlorobenzo[d]oxazol-2-yl)oxy)-3- fluorophenyl)sulfonyl)-2-(hydroxycarbamoyl)- 3,8-diazabicyclo[3.2.1]octane-8-carboxylate |
| 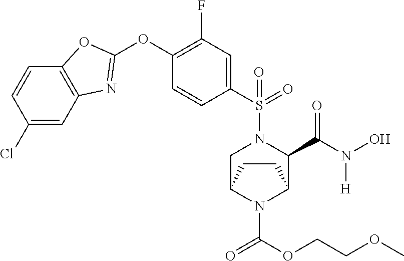 [SMILES: COCCOC(=O)N1[C@@H]2CC[C@H]1[C@H](C(=O)NO)N(S(=O)(=O)c1ccc(Oc3nc4cc(Cl)ccc4o3)c(F)c1)C2] | 2-methoxyethyl (1S,2R,5R)-3-((4-((5- chlorobenzo[d]oxazol-2-yl)oxy)-3- fluorophenyl)sulfonyl)-2-(hydroxycarbamoyl)- 3,8-diazabicyclo[3.2.1]octane-8-carboxylate |
| 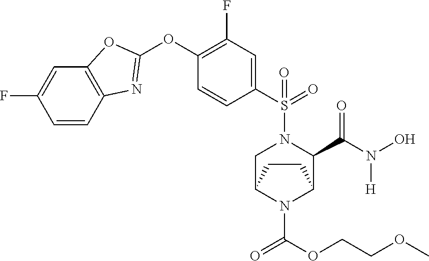 [SMILES: COCCOC(=O)N1[C@@H]2CC[C@H]1[C@H](C(=O)NO)N(S(=O)(=O)c1ccc(Oc3nc4ccc(F)cc4o3)c(F)c1)C2] | 2-methoxyethyl (1S,2R,5R)-3-((3-fluoro-4-((6- fluorobenzo[d]oxazol-2- yl)oxy)phenyl)sulfonyl)-2- (hydroxycarbamoyl)-3,8- diazabicyclo[3.2.1]octane-8-carboxylate |
| 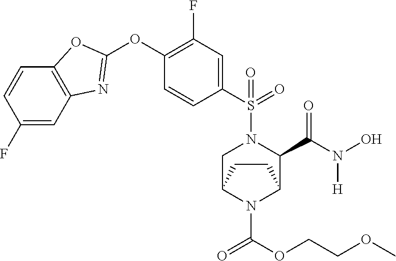 [SMILES: COCCOC(=O)N1[C@@H]2CC[C@H]1[C@H](C(=O)NO)N(S(=O)(=O)c1ccc(Oc3nc4cc(F)ccc4o3)c(F)c1)C2] | 2-methoxyethyl (1S,2R,5R)-3-((3-fluoro-4-((5- fluorobenzo[d]oxazol-2- yl)oxy)phenyl)sulfonyl)-2- (hydroxycarbamoyl)-3,8- diazabicyclo[3.2.1]octane-8-carboxylate |
| 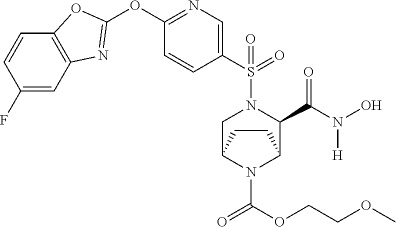 [SMILES: COCCOC(=O)N1[C@@H]2CC[C@H]1[C@H](C(=O)NO)N(S(=O)(=O)c1ccc(Oc3nc4cc(F)ccc4o3)nc1)C2] | 2-methoxyethyl (1S,2R,5R)-3-((6- (benzo[d]oxazol-2-yloxy)pyridin-3- yl)sulfonyl)-2-(hydroxycarbamoyl)-3,8- diazabicyclo[3.2.1]octane-8-carboxylate |

or a pharmaceutically acceptable salt or solvate thereof.

55. A pharmaceutical composition comprising a pharmaceutically acceptable diluent, excipient or binder, and a compound of claim 37, or a pharmaceutically acceptable salt or solvate thereof.

56. A method of treating a disease, disorder or condition selected from:
cardiovascular disease, heart failure, congestive heart failure, heart failure with reduced ejection fraction, heart failure with reserved ejection fraction, impaired cardiac contractility, age-related cardiac hypertrophy, inflammation and fibrosis, viral myocarditis, COVID-19 myocarditis, COVID-19 related myocardial fibrosis, pressure overload hypertrophy, myocardial fibrosis, myocardial infarction, myocardial ischemia/reperfusion injury, pathological remodeling of myocardium, ECM remodeling following myocardial injury, radiation myocarditis, radiation myocardial fibrosis, chemotherapy cardiomyopathy, vessel rarefaction, aortic valve sclerosis, calcific aortic valve stenosis, aortic aneurism, abdominal aorta aneurism, giant cell arteritis, age-associated arterial fibrosis, pulmonary hypertension, and right ventricle hypertrophy, idiopathic pulmonary fibrosis, acute lung injury (ALI), acute respiratory distress syndrome (ARDS), Hermansky-Pudlak syndrome (HPS), chronic obstructive pulmonary disease (COPD), emphysema, polycystic kidney disease, membranous nephropathy, diabetic nephropathy, acute kidney injury, glomerulonephritis, inherited kidney disease, and chronic allograft nephropathy, focal segmental glomerulosclerosis, minimal change disease, human immunodeficiency virus-associated nephropathy, anti-neutrophil cytoplasmic antibody-associated vasculitis, lupus nephritis, IgA nephropathy, Henoch-Schoenlein purpura, and postinfectious glomerulonephritis, membranoproliferative glomerulonephritis, cisplatin-induced renal injury, tubular injury following sepsis], acute ischemic kidney injury, contrast-induced kidney injury, acute tubular injury after ischemia and reperfusion, end-stage renal disease, tubulointerstitial fibrosis, alcoholic liver disease, non-alcoholic fatty liver disease, non-alcoholic steatohepatitis, steatosis, cirrhosis, hepatic ischemia and reperfusion injury, viral hepatitis, drug-induced liver injury, primary biliary cholangitis, primary sclerosing cholangitis, hemochromatosis, Wilson's disease, acute liver failure, biliary atresia, and scleroderma pigmentosum,
in a mammal in need thereof, comprising administering to the mammal in need thereof a therapeutically effective amount of a compound of claim 37, or a pharmaceutically acceptable salt or solvate thereof.
